SEQUENCE LISTING (123 sequences)

SEQ ID NO: 1 - DNA, 665 bp, Artificial Sequence
  Synthetic
gagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagcttgatatcatgtgtctgagcctgcatgtttgatggtgtctggatgcaagcagaaggggtggaagagcttgcctggagagatacagctgggtcagtaggactgggacaggcagctggagaattgccatgtagatgttcatacaatcgtcaaatcatgaaggctggaaaagccctccaagatccccaagaccaaccccaacccacccaccgtgcccactggccatgtccctcagtgccacatccccacagttcttcatcacctccagggacggtgacccccccacctccgtgggcagctgtgccactgcagcaccgctctttggagaaggtaaatcttgctaaatccagcccgaccctcccctggcacaacgtaaggccattatctctcatccaactccaggacggagtcagtgaggatggggct

SEQ ID NO: 2 - DNA, 665 bp, Artificial Sequence
  Synthetic
agccccatcctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatccagacaccatcaaacatgcaggctcagacacatgatatcaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctc

SEQ ID NO: 3 - DNA, 665 bp, Artificial Sequence
  Synthetic
agccccatcctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatcctgacaccatcaaacatgcaggctcagacacatgatatcaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctc

SEQ ID NO: 4 - DNA, 665 bp, Artificial Sequence
  Synthetic
agccccatcctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatcctgacaccatcaaacatgcaggctcagacacatgatatcaagctttttccccgtatcccccctggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctc

SEQ ID NO: 5 - DNA, 665 bp, Artificial Sequence
  Synthetic
agccccatcctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatcctgacaccatcaaacatgcaggctcagacacatgatatcaagctttttccccgtatccccccaggtgtctgctggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctc

SEQ ID NO: 6 - DNA, 665 bp, Artificial Sequence
  Synthetic
agccccatcctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatcctgacaccatcaaacatgcaggctcagacacatgatatcaagctttttccccgtatcccccctggtgtctgctggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctc

SEQ ID NO: 7 - DNA, 665 bp, Artificial Sequence
  Synthetic
agccccatcctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatccagacaccatcaaacatgcaggctcagacacatgatatcaagctttttccccgtatcccccctggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctc

SEQ ID NO: 8 - DNA, 665 bp, Artificial Sequence
  Synthetic
agccccatcctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatccagacaccatcaaacatgcaggctcagacacatgatatcaagctttttccccgtatcccccctggtgtctgctggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctc

SEQ ID NO: 9 - DNA, 665 bp, Artificial Sequence
  Synthetic
agccccatcctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatccagacaccatcaaacatgcaggctcagacacatgatatcaagctttttccccgtatccccccaggtgtctgctggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctc

SEQ ID NO: 10 - DNA, 649 bp, Artificial Sequence
  Synthetic
acggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagctttaggcttgtgtctgagcctgcatgtttgatggtgtctggatgcaagcagaaggggtggaagagcttgcctggagagatacagctgggtcagtaggactgggacaggcagctggagaattgccatgtagatgttcatacaatcgtcaaatcatgaaggctggaaaagccctccaagatccccaagaccaaccccaacccacccaccgtgcccactggccatgtccctcagtgccacatccccacagttcttcatcacctccagggacggtgacccccccacctccgtgggcagctgtgccactgcagcaccgctctttggagaaggtaaatcttgctaaatccagcccgaccctcccctggcacaacgtaaggccattatctctcatccaactccaggacggagtcagtgag

SEQ ID NO: 11 - DNA, 649 bp, Artificial Sequence
  Synthetic
ctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatccagacaccatcaaacatgcaggctcagacacaagcctaaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgt

SEQ ID NO: 12 - DNA, 649 bp, Artificial Sequence
  Synthetic
ctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatcctgacaccatcaaacatgcaggctcagacacaagcctaaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgt

SEQ ID NO: 13 - DNA, 649 bp, Artificial Sequence
  Synthetic
ctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatcctgacaccatcaaacatgcaggctcagacacaagcctaaagctttttccccgtatcccccctggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgt

SEQ ID NO: 14 - DNA, 649 bp, Artificial Sequence
  Synthetic
ctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatcctgacaccatcaaacatgcaggctcagacacaagcctaaagctttttccccgtatccccccaggtgtctgctggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgt

SEQ ID NO: 15 - DNA, 649 bp, Artificial Sequence
  Synthetic
ctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatcctgacaccatcaaacatgcaggctcagacacaagcctaaagctttttccccgtatcccccctggtgtctgctggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgt

SEQ ID NO: 16 - DNA, 649 bp, Artificial Sequence
  Synthetic
ctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatccagacaccatcaaacatgcaggctcagacacaagcctaaagctttttccccgtatcccccctggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgt

SEQ ID NO: 17 - DNA, 649 bp, Artificial Sequence
  Synthetic
ctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatccagacaccatcaaacatgcaggctcagacacaagcctaaagctttttccccgtatcccccctggtgtctgctggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgt

SEQ ID NO: 18 - DNA, 649 bp, Artificial Sequence
  Synthetic
ctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatccagacaccatcaaacatgcaggctcagacacaagcctaaagctttttccccgtatccccccaggtgtctgctggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgt

SEQ ID NO: 19 - DNA, 655 bp, Artificial Sequence
  Synthetic
aagatctgctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaatgtgtctgagcctgcatgtttgatggtgtctggatgcaagcagaaggggtggaagagcttgcctggagagatacagctgggtcagtaggactgggacaggcagctggagaattgccatgtagatgttcatacaatcgtcaaatcatgaaggctggaaaagccctccaagatccccaagaccaaccccaacccacccaccgtgcccactggccatgtccctcagtgccacatccccacagttcttcatcacctccagggacggtgacccccccacctccgtgggcagctgtgccactgcagcaccgctctttggagaaggtaaatcttgctaaatccagcccgaccctcccctggcacaacgtaaggccattatctctcatccaactccaggacggagtcagtgagaatatt

SEQ ID NO: 20 - DNA, 655 bp, Artificial Sequence
  Synthetic
aatattctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatccagacaccatcaaacatgcaggctcagacacattttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagcagatctt

SEQ ID NO: 21 - DNA, 655 bp, Artificial Sequence
  Synthetic
aatattctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatcctgacaccatcaaacatgcaggctcagacacattttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagcagatctt

SEQ ID NO: 22 - DNA, 655 bp, Artificial Sequence
  Synthetic
aatattctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatcctgacaccatcaaacatgcaggctcagacacattttccccgtatcccccctggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagcagatctt

SEQ ID NO: 23 - DNA, 655 bp, Artificial Sequence
  Synthetic
aatattctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatcctgacaccatcaaacatgcaggctcagacacattttccccgtatccccccaggtgtctgctggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagcagatctt

SEQ ID NO: 24 - DNA, 655 bp, Artificial Sequence
  Synthetic
aatattctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatcctgacaccatcaaacatgcaggctcagacacattttccccgtatcccccctggtgtctgctggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagcagatctt

SEQ ID NO: 25 - DNA, 655 bp, Artificial Sequence
  Synthetic
aatattctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatccagacaccatcaaacatgcaggctcagacacattttccccgtatcccccctggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagcagatctt

SEQ ID NO: 26 - DNA, 655 bp, Artificial Sequence
  Synthetic
aatattctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatccagacaccatcaaacatgcaggctcagacacattttccccgtatcccccctggtgtctgctggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagcagatctt

SEQ ID NO: 27 - DNA, 655 bp, Artificial Sequence
  Synthetic
aatattctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatccagacaccatcaaacatgcaggctcagacacattttccccgtatccccccaggtgtctgctggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagcagatctt

SEQ ID NO: 28 - DNA, 651 bp, Artificial Sequence
  Synthetic
cctgtacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaatgtgtctgagcctgcatgtttgatggtgtctggatgcaagcagaaggggtggaagagcttgcctggagagatacagctgggtcagtaggactgggacaggcagctggagaattgccatgtagatgttcatacaatcgtcaaatcatgaaggctggaaaagccctccaagatccccaagaccaaccccaacccacccaccgtgcccactggccatgtccctcagtgccacatccccacagttcttcatcacctccagggacggtgacccccccacctccgtgggcagctgtgccactgcagcaccgctctttggagaaggtaaatcttgctaaatccagcccgaccctcccctggcacaacgtaaggccattatctctcatccaactccaggacggagtcagtgaggatggggct

SEQ ID NO: 29 - DNA, 651 bp, Artificial Sequence
  Synthetic
agccccatcctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatccagacaccatcaaacatgcaggctcagacacatttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtacagg

SEQ ID NO: 30 - DNA, 651 bp, Artificial Sequence
  Synthetic
agccccatcctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatcctgacaccatcaaacatgcaggctcagacacatttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtacagg

SEQ ID NO: 31 - DNA, 651 bp, Artificial Sequence
  Synthetic
agccccatcctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatcctgacaccatcaaacatgcaggctcagacacatttccccgtatcccccctggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtacagg

SEQ ID NO: 32 - DNA, 651 bp, Artificial Sequence
  Synthetic
agccccatcctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatcctgacaccatcaaacatgcaggctcagacacatttccccgtatccccccaggtgtctgctggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtacagg

SEQ ID NO: 33 - DNA, 651 bp, Artificial Sequence
  Synthetic
agccccatcctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatcctgacaccatcaaacatgcaggctcagacacatttccccgtatcccccctggtgtctgctggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtacagg

SEQ ID NO: 34 - DNA, 651 bp, Artificial Sequence
  Synthetic
agccccatcctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatccagacaccatcaaacatgcaggctcagacacatttccccgtatcccccctggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtacagg

SEQ ID NO: 35 - DNA, 651 bp, Artificial Sequence
  Synthetic
agccccatcctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatccagacaccatcaaacatgcaggctcagacacatttccccgtatcccccctggtgtctgctggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtacagg

SEQ ID NO: 36 - DNA, 651 bp, Artificial Sequence
  Synthetic
agccccatcctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatccagacaccatcaaacatgcaggctcagacacatttccccgtatccccccaggtgtctgctggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtacagg

SEQ ID NO: 37 - DNA, 651 bp, Artificial Sequence; identical to SEQ ID NO: 28
  Synthetic

SEQ ID NO: 38 - DNA, 647 bp, Artificial Sequence
  Synthetic
agccccatcctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatccagacaccatcaaacatgcaggctcagacacatttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtg

SEQ ID NO: 39 - DNA, 647 bp, Artificial Sequence
  Synthetic
agccccatcctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatcctgacaccatcaaacatgcaggctcagacacatttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtg

SEQ ID NO: 40 - DNA, 647 bp, Artificial Sequence
  Synthetic
agccccatcctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatcctgacaccatcaaacatgcaggctcagacacatttccccgtatcccccctggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtg

SEQ ID NO: 41 - DNA, 647 bp, Artificial Sequence
  Synthetic
agccccatcctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatcctgacaccatcaaacatgcaggctcagacacatttccccgtatccccccaggtgtctgctggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtg

SEQ ID NO: 42 - DNA, 647 bp, Artificial Sequence
  Synthetic
agccccatcctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatcctgacaccatcaaacatgcaggctcagacacatttccccgtatcccccctggtgtctgctggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtg

SEQ ID NO: 43 - DNA, 647 bp, Artificial Sequence
  Synthetic
agccccatcctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatccagacaccatcaaacatgcaggctcagacacatttccccgtatcccccctggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtg

SEQ ID NO: 44 - DNA, 647 bp, Artificial Sequence
  Synthetic
agccccatcctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatccagacaccatcaaacatgcaggctcagacacatttccccgtatcccccctggtgtctgctggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtg

SEQ ID NO: 45 - DNA, 647 bp, Artificial Sequence
  Synthetic
agccccatcctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatccagacaccatcaaacatgcaggctcagacacatttccccgtatccccccaggtgtctgctggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtg

SEQ ID NO: 46 - DNA, 641 bp, Artificial Sequence
  Synthetic
cggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaatgtgtctgagcctgcatgtttgatggtgtctggatgcaagcagaaggggtggaagagcttgcctggagagatacagctgggtcagtaggactgggacaggcagctggagaattgccatgtagatgttcatacaatcgtcaaatcatgaaggctggaaaagccctccaagatccccaagaccaaccccaacccacccaccgtgcccactggccatgtccctcagtgccacatccccacagttcttcatcacctccagggacggtgacccccccacctccgtgggcagctgtgccactgcagcaccgctctttggagaaggtaaatcttgctaaatccagcccgaccctcccctggcacaacgtaaggccattatctctcatccaactccaggacggagtcagtgagaata

SEQ ID NO: 47 - DNA, 641 bp, Artificial Sequence
  Synthetic
tattctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatccagacaccatcaaacatgcaggctcagacacattttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccg

SEQ ID NO: 48 - DNA, 641 bp, Artificial Sequence
  Synthetic
tattctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatcctgacaccatcaaacatgcaggctcagacacattttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccg

SEQ ID NO: 49 - DNA, 641 bp, Artificial Sequence
  Synthetic
tattctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatcctgacaccatcaaacatgcaggctcagacacattttccccgtatcccccctggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccg

SEQ ID NO: 50 - DNA, 641 bp, Artificial Sequence
  Synthetic
tattctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatcctgacaccatcaaacatgcaggctcagacacattttccccgtatccccccaggtgtctgctggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccg

SEQ ID NO: 51 - DNA, 641 bp, Artificial Sequence
  Synthetic
tattctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatcctgacaccatcaaacatgcaggctcagacacattttccccgtatcccccctggtgtctgctggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccg

SEQ ID NO: 52 - DNA, 641 bp, Artificial Sequence
  Synthetic
tattctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatccagacaccatcaaacatgcaggctcagacacattttccccgtatcccccctggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccg

SEQ ID NO: 53 - DNA, 641 bp, Artificial Sequence
  Synthetic
tattctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatccagacaccatcaaacatgcaggctcagacacattttccccgtatcccccctggtgtctgctggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccg

SEQ ID NO: 54 - DNA, 641 bp, Artificial Sequence
  Synthetic
tattctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatccagacaccatcaaacatgcaggctcagacacattttccccgtatccccccaggtgtctgctggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccg

SEQ ID NO: 55 - DNA, 9651 bp, Artificial Sequence
  Synthetic
ggccgcctcggccaaacagcccttgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccagtccctatcagtgatagagaaaagtgaaagtcgagtttaccactccctatcagtgatagagaaaagtgaaagtcgagtttaccactccctatcagtgatagagaaaagtgaaagtcgagctcgccatgggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatactggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcaggatcttcagacctggaaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccataaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggaccgagctcaagcttcgaagcgatcgcacgcgtcaaaaaaggatatgcccttgactatgtcggacaaatagtcaagggcatatcctgaggtacccaggcggcgcacaagctatataaacctgaaggaaatctcaactttacacttaggtcaagttacttatcgtactagagcttcagcaggaaatttaactaaaatctaatttaaccagcatagcaaatatcatttattcccaaaatgctaaagtttgagataaacggacttgatttccggctgttttgacactatccagaatgccttgcagatgggtggggcatgctaaatactgcacgtcgatacgcgtggatccgaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcggcgcgccaattcaagcgagaagacaagggcagccgccaccatgagtgggggcccaatgggaggaaggcccgggggccgaggagcaccagcggttcagcagaacataccctccaccctcctccaggaccacgagaaccagcgactctttgagatgcttggacgaaaatgcttgacgctggccactgcagttgttcagctgtacctggcgctgccccctggagctgagcactggaccaaggagcattgtggggctgtgtgcttcgtgaaggataacccccagaagtcctacttcatccgcctttacggccttcaggctggtcggctgctctgggaacaggagctgtactcacagcttgtctactccacccccacccccttcttccacaccttcgctggagatgactgccaagcggggctgaactttgcagacgaggacgaggcccaggccttccgggcactcgtgcaggagaagatacaaaaaaggaatcagaggcaaagtggagacagacgccagctacccccaccaccaacaccagccaatgaagagagaagaggagggctcccacccctgcccctgcatccaggtggagaccaaggaggccctccagtgggtccgctctccctggggctggcgacagtggacatccagaaccctgacatcacgagttcacgataccgtgggctcccagcacctggacctagcccagctgataagaaacgctcagggaagaagaagatcagcaaagctgatattggtgcacccagtggattcaagcatgtcagccacgtggggtgggacccccagaatggatttgacgtgaacaacctcgacccagatctgcggagtctgttctccagggcaggaatcagcgaggcccagctcaccgacgccgagacctctaaacttatctacgacttcattgaggaccagggtgggctggaggctgtgcggcaggagatgaggcgccaggagccacttccgccgcccccaccgccatctcgaggagggaaccagctcccccggccccctattgtggggggtaacaagggtcgttctggtccactgccccctgtacctttggggattgccccacccccaccaacaccccggggacccccacccccaggccgagggggtcctccaccaccaccccctccagctactggacgttctggaccactgccccctccaccccctggagctggtgggccacccatgccaccaccaccgccaccaccgccaccgccgcccagctccgggaatggaccagcccctcccccactccctcctgctctggtgcctgccgggggcctggcccctggtgggggtcggggagcgcttttggatcaaatccggcagggaattcagctgaacaagacccctggggccccagagagctcagcgctgcagccaccacctcagagctcagagggactggtgggggccctgatgcacgtgatgcagaagagaagcagagccatccactcctccgacgaaggggaggaccaggctggcgatgaagatgaagatgatgaatgggatgactgataactagtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgttcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcacgtacgaccggtgcggccgcatcgatgccgtagtacctttaagaccaatgacttacaaggcagctgtagatcttagccactttttaaaagaaaaggggggactggaagggctaattcactcccaaagaagacaagagccccatcctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatccagacaccatcaaacatgcaggctcagacacatgatatcaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctccccagatctgctttttgcctgtactgggtctctctggttagaccagatctgagcctgggagctctctggctaactagggaacccactgcttaagcctcaataaagcttcagctgctcgagctagcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattc

SEQ ID NO: 56 - DNA, 9653 bp, Artificial Sequence
  Synthetic
ggccgcctcggccaaacagcccttgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccagtccctatcagtgatagagaaaagtgaaagtcgagtttaccactccctatcagtgatagagaaaagtgaaagtcgagtttaccactccctatcagtgatagagaaaagtgaaagtcgagctcgccatgggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatactggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcaggatcttcagacctggaaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccataaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggaccgagctcaagcttcgaagcgatcgcacgcgtcaaaaaaggatatgcccttgactatgtcggacaaatagtcaagggcatatcctgaggtacccaggcggcgcacaagctatataaacctgaaggaaatctcaactttacacttaggtcaagttacttatcgtactagagcttcagcaggaaatttaactaaaatctaatttaaccagcatagcaaatatcatttattcccaaaatgctaaagtttgagataaacggacttgatttccggctgttttgacactatccagaatgccttgcagatgggtggggcatgctaaatactgcacgtcgatacgcgtggatccgaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcggcgcgccaattcaagcgagaagacaagggcagccgccaccatgagtgggggcccaatgggaggaaggcccgggggccgaggagcaccagcggttcagcagaacataccctccaccctcctccaggaccacgagaaccagcgactctttgagatgcttggacgaaaatgcttgacgctggccactgcagttgttcagctgtacctggcgctgccccctggagctgagcactggaccaaggagcattgtggggctgtgtgcttcgtgaaggataacccccagaagtcctacttcatccgcctttacggccttcaggctggtcggctgctctgggaacaggagctgtactcacagcttgtctactccacccccacccccttcttccacaccttcgctggagatgactgccaagcggggctgaactttgcagacgaggacgaggcccaggccttccgggcactcgtgcaggagaagatacaaaaaaggaatcagaggcaaagtggagacagacgccagctacccccaccaccaacaccagccaatgaagagagaagaggagggctcccacccctgcccctgcatccaggtggagaccaaggaggccctccagtgggtccgctctccctggggctggcgacagtggacatccagaaccctgacatcacgagttcacgataccgtgggctcccagcacctggacctagcccagctgataagaaacgctcagggaagaagaagatcagcaaagctgatattggtgcacccagtggattcaagcatgtcagccacgtggggtgggacccccagaatggatttgacgtgaacaacctcgacccagatctgcggagtctgttctccagggcaggaatcagcgaggcccagctcaccgacgccgagacctctaaacttatctacgacttcattgaggaccagggtgggctggaggctgtgcggcaggagatgaggcgccaggagccacttccgccgcccccaccgccatctcgaggagggaaccagctcccccggccccctattgtggggggtaacaagggtcgttctggtccactgccccctgtacctttggggattgccccacccccaccaacaccccggggacccccacccccaggccgagggggtcctccaccaccaccccctccagctactggacgttctggaccactgccccctccaccccctggagctggtgggccacccatgccaccaccaccgccaccaccgccaccgccgcccagctccgggaatggaccagcccctcccccactccctcctgctctggtgcctgccgggggcctggcccctggtgggggtcggggagcgcttttggatcaaatccggcagggaattcagctgaacaagacccctggggccccagagagctcagcgctgcagccaccacctcagagctcagagggactggtgggggccctgatgcacgtgatgcagaagagaagcagagccatccactcctccgacgaaggggaggaccaggctggcgatgaagatgaagatgatgaatgggatgactgataactagtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcacgtacgaccggtgcggccgcatcgatgccgtagtacctttaagaccaatgacttacaaggcagctgtagatcttagccactttttaaaagaaaaggggggactggaagggctaattcactcccaaagaagacaagatagccccatcctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatccagacaccatcaaacatgcaggctcagacacatgatatcaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctccccagatctgctttttgcctgtactgggtctctctggttagaccagatctgagcctgggagctctctggctaactagggaacccactgcttaagcctcaataaagcttcagctgctcgagctagcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattc

SEQ ID NO: 57 - DNA, 9653 bp, Artificial Sequence
  Synthetic
ggccgcctcggccaaacagcccttgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccagtccctatcagtgatagagaaaagtgaaagtcgagtttaccactccctatcagtgatagagaaaagtgaaagtcgagtttaccactccctatcagtgatagagaaaagtgaaagtcgagctcgccatgggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatactggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcaggatcttcagacctggaaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccataaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggaccgagctcaagcttcgaagcgatcgcacgcgtcaaaaaaggatatgcccttgactatgtcggacaaatagtcaagggcatatcctgaggtacccaggcggcgcacaagctatataaacctgaaggaaatctcaactttacacttaggtcaagttacttatcgtactagagcttcagcaggaaatttaactaaaatctaatttaaccagcatagcaaatatcatttattcccaaaatgctaaagtttgagataaacggacttgatttccggctgttttgacactatccagaatgccttgcagatgggtggggcatgctaaatactgcacgtcgatacgcgtggatccgaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcggcgcgccaattcaagcgagaagacaagggcagccgccaccatgagtgggggcccaatgggaggaaggcccgggggccgaggagcaccagcggttcagcagaacataccctccaccctcctccaggaccacgagaaccagcgactctttgagatgcttggacgaaaatgcttgacgctggccactgcagttgttcagctgtacctggcgctgccccctggagctgagcactggaccaaggagcattgtggggctgtgtgcttcgtgaaggataacccccagaagtcctacttcatccgcctttacggccttcaggctggtcggctgctctgggaacaggagctgtactcacagcttgtctactccacccccacccccttcttccacaccttcgctggagatgactgccaagcggggctgaactttgcagacgaggacgaggcccaggccttccgggcactcgtgcaggagaagatacaaaaaaggaatcagaggcaaagtggagacagacgccagctacccccaccaccaacaccagccaatgaagagagaagaggagggctcccacccctgcccctgcatccaggtggagaccaaggaggccctccagtgggtccgctctccctggggctggcgacagtggacatccagaaccctgacatcacgagttcacgataccgtgggctcccagcacctggacctagcccagctgataagaaacgctcagggaagaagaagatcagcaaagctgatattggtgcacccagtggattcaagcatgtcagccacgtggggtgggacccccagaatggatttgacgtgaacaacctcgacccagatctgcggagtctgttctccagggcaggaatcagcgaggcccagctcaccgacgccgagacctctaaacttatctacgacttcattgaggaccagggtgggctggaggctgtgcggcaggagatgaggcgccaggagccacttccgccgcccccaccgccatctcgaggagggaaccagctcccccggccccctattgtggggggtaacaagggtcgttctggtccactgccccctgtacctttggggattgccccacccccaccaacaccccggggacccccacccccaggccgagggggtcctccaccaccaccccctccagctactggacgttctggaccactgccccctccaccccctggagctggtgggccacccatgccaccaccaccgccaccaccgccaccgccgcccagctccgggaatggaccagcccctcccccactccctcctgctctggtgcctgccgggggcctggcccctggtgggggtcggggagcgcttttggatcaaatccggcagggaattcagctgaacaagacccctggggccccagagagctcagcgctgcagccaccacctcagagctcagagggactggtgggggccctgatgcacgtgatgcagaagagaagcagagccatccactcctccgacgaaggggaggaccaggctggcgatgaagatgaagatgatgaatgggatgactgataactagtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcacgtacgaccggtgcggccgcatcgatgccgtagtacctttaagaccaatgacttacaaggcagctgtagatcttagccactttttaaaagaaaaggggggactggaagggctaattcactcccaaagaagacaagatagccccatcctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatcctgacaccatcaaacatgcaggctcagacacatgatatcaagctttttccccgtatcccccctggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctccccagatctgctttttgcctgtactgggtctctctggttagaccagatctgagcctgggagctctctggctaactagggaacccactgcttaagcctcaataaagcttcagctgctcgagctagcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattc

SEQ ID NO: 58 - DNA, 9653 bp, Artificial Sequence
  Synthetic
ggccgcctcggccaaacagcccttgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccagtccctatcagtgatagagaaaagtgaaagtcgagtttaccactccctatcagtgatagagaaaagtgaaagtcgagtttaccactccctatcagtgatagagaaaagtgaaagtcgagctcgccatgggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatactggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcaggatcttcagacctggaaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccataaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggaccgagctcaagcttcgaagcgatcgcacgcgtcaaaaaaggatatgcccttgactatgtcggacaaatagtcaagggcatatcctgaggtacccaggcggcgcacaagctatataaacctgaaggaaatctcaactttacacttaggtcaagttacttatcgtactagagcttcagcaggaaatttaactaaaatctaatttaaccagcatagcaaatatcatttattcccaaaatgctaaagtttgagataaacggacttgatttccggctgttttgacactatccagaatgccttgcagatgggtggggcatgctaaatactgcacgtcgatacgcgtggatccgaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcggcgcgccaattcaagcgagaagacaagggcagccgccaccatgagtgggggcccaatgggaggaaggcccgggggccgaggagcaccagcggttcagcagaacataccctccaccctcctccaggaccacgagaaccagcgactctttgagatgcttggacgaaaatgcttgacgctggccactgcagttgttcagctgtacctggcgctgccccctggagctgagcactggaccaaggagcattgtggggctgtgtgcttcgtgaaggataacccccagaagtcctacttcatccgcctttacggccttcaggctggtcggctgctctgggaacaggagctgtactcacagcttgtctactccacccccacccccttcttccacaccttcgctggagatgactgccaagcggggctgaactttgcagacgaggacgaggcccaggccttccgggcactcgtgcaggagaagatacaaaaaaggaatcagaggcaaagtggagacagacgccagctacccccaccaccaacaccagccaatgaagagagaagaggagggctcccacccctgcccctgcatccaggtggagaccaaggaggccctccagtgggtccgctctccctggggctggcgacagtggacatccagaaccctgacatcacgagttcacgataccgtgggctcccagcacctggacctagcccagctgataagaaacgctcagggaagaagaagatcagcaaagctgatattggtgcacccagtggattcaagcatgtcagccacgtggggtgggacccccagaatggatttgacgtgaacaacctcgacccagatctgcggagtctgttctccagggcaggaatcagcgaggcccagctcaccgacgccgagacctctaaacttatctacgacttcattgaggaccagggtgggctggaggctgtgcggcaggagatgaggcgccaggagccacttccgccgcccccaccgccatctcgaggagggaaccagctcccccggccccctattgtggggggtaacaagggtcgttctggtccactgccccctgtacctttggggattgccccacccccaccaacaccccggggacccccacccccaggccgagggggtcctccaccaccaccccctccagctactggacgttctggaccactgccccctccaccccctggagctggtgggccacccatgccaccaccaccgccaccaccgccaccgccgcccagctccgggaatggaccagcccctcccccactccctcctgctctggtgcctgccgggggcctggcccctggtgggggtcggggagcgcttttggatcaaatccggcagggaattcagctgaacaagacccctggggccccagagagctcagcgctgcagccaccacctcagagctcagagggactggtgggggccctgatgcacgtgatgcagaagagaagcagagccatccactcctccgacgaaggggaggaccaggctggcgatgaagatgaagatgatgaatgggatgactgataactagtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcacgtacgaccggtgcggccgcatcgatgccgtagtacctttaagaccaatgacttacaaggcagctgtagatcttagccactttttaaaagaaaaggggggactggaagggctaattcactcccaaagaagacaagatggggagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagcttgatatcatgtgtctgagcctgcatgtttgatggtgtctggatgcaagcagaaggggtggaagagcttgcctggagagatacagctgggtcagtaggactgggacaggcagctggagaattgccatgtagatgttcatacaatcgtcaaatcatgaaggctggaaaagccctccaagatccccaagaccaaccccaacccacccaccgtgcccactggccatgtccctcagtgccacatccccacagttcttcatcacctccagggacggtgacccccccacctccgtgggcagctgtgccactgcagcaccgctctttggagaaggtaaatcttgctaaatccagcccgaccctcccctggcacaacgtaaggccattatctctcatccaactccaggacggagtcagtgaggatggggctagatctgctttttgcctgtactgggtctctctggttagaccagatctgagcctgggagctctctggctaactagggaacccactgcttaagcctcaataaagcttcagctgctcgagctagcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattc

SEQ ID NO: 59 - DNA, 9653 bp, Artificial Sequence
  Synthetic
ggccgcctcggccaaacagcccttgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccagtccctatcagtgatagagaaaagtgaaagtcgagtttaccactccctatcagtgatagagaaaagtgaaagtcgagtttaccactccctatcagtgatagagaaaagtgaaagtcgagctcgccatgggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatactggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcaggatcttcagacctggaaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccataaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggaccgagctcaagcttcgaagcgatcgcacgcgtcaaaaaaggatatgcccttgactatgtcggacaaatagtcaagggcatatcctgaggtacccaggcggcgcacaagctatataaacctgaaggaaatctcaactttacacttaggtcaagttacttatcgtactagagcttcagcaggaaatttaactaaaatctaatttaaccagcatagcaaatatcatttattcccaaaatgctaaagtttgagataaacggacttgatttccggctgttttgacactatccagaatgccttgcagatgggtggggcatgctaaatactgcacgtcgatacgcgtggatccgaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcggcgcgccaattcaagcgagaagacaagggcagccgccaccatgagtgggggcccaatgggaggaaggcccgggggccgaggagcaccagcggttcagcagaacataccctccaccctcctccaggaccacgagaaccagcgactctttgagatgcttggacgaaaatgcttgacgctggccactgcagttgttcagctgtacctggcgctgccccctggagctgagcactggaccaaggagcattgtggggctgtgtgcttcgtgaaggataacccccagaagtcctacttcatccgcctttacggccttcaggctggtcggctgctctgggaacaggagctgtactcacagcttgtctactccacccccacccccttcttccacaccttcgctggagatgactgccaagcggggctgaactttgcagacgaggacgaggcccaggccttccgggcactcgtgcaggagaagatacaaaaaaggaatcagaggcaaagtggagacagacgccagctacccccaccaccaacaccagccaatgaagagagaagaggagggctcccacccctgcccctgcatccaggtggagaccaaggaggccctccagtgggtccgctctccctggggctggcgacagtggacatccagaaccctgacatcacgagttcacgataccgtgggctcccagcacctggacctagcccagctgataagaaacgctcagggaagaagaagatcagcaaagctgatattggtgcacccagtggattcaagcatgtcagccacgtggggtgggacccccagaatggatttgacgtgaacaacctcgacccagatctgcggagtctgttctccagggcaggaatcagcgaggcccagctcaccgacgccgagacctctaaacttatctacgacttcattgaggaccagggtgggctggaggctgtgcggcaggagatgaggcgccaggagccacttccgccgcccccaccgccatctcgaggagggaaccagctcccccggccccctattgtggggggtaacaagggtcgttctggtccactgccccctgtacctttggggattgccccacccccaccaacaccccggggacccccacccccaggccgagggggtcctccaccaccaccccctccagctactggacgttctggaccactgccccctccaccccctggagctggtgggccacccatgccaccaccaccgccaccaccgccaccgccgcccagctccgggaatggaccagcccctcccccactccctcctgctctggtgcctgccgggggcctggcccctggtgggggtcggggagcgcttttggatcaaatccggcagggaattcagctgaacaagacccctggggccccagagagctcagcgctgcagccaccacctcagagctcagagggactggtgggggccctgatgcacgtgatgcagaagagaagcagagccatccactcctccgacgaaggggaggaccaggctggcgatgaagatgaagatgatgaatgggatgactgataactagtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcacgtacgaccggtgcggccgcatcgatgccgtagtacctttaagaccaatgacttacaaggcagctgtagatcttagccactttttaaaagaaaaggggggactggaagggctaattcactcccaaagaagacaagatagccccatcctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatcctgacaccatcaaacatgcaggctcagacacatgatatcaagctttttccccgtatcccccctggtgtctgctggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctccccagatctgctttttgcctgtactgggtctctctggttagaccagatctgagcctgggagctctctggctaactagggaacccactgcttaagcctcaataaagcttcagctgctcgagctagcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattc

SEQ ID NO: 60 - DNA, 20 bp, Artificial Sequence
  Synthetic
ttgcatccagacaccatcaa

SEQ ID NO: 61 - DNA, 20 bp, Artificial Sequence
  Synthetic
atccccccaggtgtctgcag

SEQ ID NO: 62 - DNA, 20 bp, Artificial Sequence
  Synthetic
gtgtctgcaggctcaaagag

SEQ ID NO: 63 - DNA, 20 bp, Artificial Sequence
  Synthetic
ttgcatcctgacaccatcaa

SEQ ID NO: 64 - DNA, 20 bp, Artificial Sequence
  Synthetic
atcccccctggtgtctgcag

SEQ ID NO: 65 - DNA, 20 bp, Artificial Sequence
  Synthetic
gtgtctgctggctcaaagag

SEQ ID NO: 66 - DNA, 46 bp, Artificial Sequence
  Synthetic
aggatatgcccttgactatttgtccgacatagtcaagggcatatcc

SEQ ID NO: 67 - DNA, 52 bp, Artificial Sequence
  Synthetic
aggatatgcccttgactatttgtccgacatagtcaagggcatatcctttttt

SEQ ID NO: 68 - DNA, 47 bp, Artificial Sequence
  Synthetic
aggatatgcccttgactatttgtccgacatagtcaagggcatatcct

SEQ ID NO: 69 - DNA, 249 bp, Artificial Sequence
  Synthetic
atcgacgtgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggtacctc

SEQ ID NO: 70 - DNA, 249 bp, Artificial Sequence
  Synthetic
atcgacgtgcagtcgggctactgccccacccatagtaccggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggtacctc

SEQ ID NO: 71 - DNA, 243 bp, Artificial Sequence
  Synthetic
ctgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagcaacttgacctaagtgtaaagttgagatttccttcaggtttatatagcttgtgcgccgcctgggtacctc

SEQ ID NO: 72 - DNA, 347 bp, Artificial Sequence
  Synthetic
gaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatc

SEQ ID NO: 73 - DNA, 1506 bp, Artificial Sequence
  Synthetic
atgagtgggggcccaatgggaggaaggcccgggggccgaggagcaccagcggttcagcagaacataccctccaccctcctccaggaccacgagaaccagcgactctttgagatgcttggacgaaaatgcttgacgctggccactgcagttgttcagctgtacctggcgctgccccctggagctgagcactggaccaaggagcattgtggggctgtgtgcttcgtgaaggataacccccagaagtcctacttcatccgcctttacggccttcaggctggtcggctgctctgggaacaggagctgtactcacagcttgtctactccacccccacccccttcttccacaccttcgctggagatgactgccaagcggggctgaactttgcagacgaggacgaggcccaggccttccgggcactcgtgcaggagaagatacaaaaaaggaatcagaggcaaagtggagacagacgccagctacccccaccaccaacaccagccaatgaagagagaagaggagggctcccacccctgcccctgcatccaggtggagaccaaggaggccctccagtgggtccgctctccctggggctggcgacagtggacatccagaaccctgacatcacgagttcacgataccgtgggctcccagcacctggacctagcccagctgataagaaacgctcagggaagaagaagatcagcaaagctgatattggtgcacccagtggattcaagcatgtcagccacgtggggtgggacccccagaatggatttgacgtgaacaacctcgacccagatctgcggagtctgttctccagggcaggaatcagcgaggcccagctcaccgacgccgagacctctaaacttatctacgacttcattgaggaccagggtgggctggaggctgtgcggcaggagatgaggcgccaggagccacttccgccgcccccaccgccatctcgaggagggaaccagctcccccggccccctattgtggggggtaacaagggtcgttctggtccactgccccctgtacctttggggattgccccacccccaccaacaccccggggacccccacccccaggccgagggggtcctccaccaccaccccctccagctactggacgttctggaccactgccccctccaccccctggagctggtgggccacccatgccaccaccaccgccaccaccgccaccgccgcccagctccgggaatggaccagcccctcccccactccctcctgctctggtgcctgccgggggcctggcccctggtgggggtcggggagcgcttttggatcaaatccggcagggaattcagctgaacaagacccctggggccccagagagctcagcgctgcagccaccacctcagagctcagagggactggtgggggccctgatgcacgtgatgcagaagagaagcagagccatccactcctccgacgaaggggaggaccaggctggcgatgaagatgaagatgatgaatgggatgac

SEQ ID NO: 74 - DNA, 1506 bp, Artificial Sequence
  Synthetic
atgagtgggggcccaatgggaggaaggcccgggggccgaggagcaccagcggttcagcagaacataccctccaccctcctccaggaccacgagaaccagcgactctttgagatgcttggacgaaaatgcttgacgctggccactgcagttgttcagctgtacctggcgctgccccctggagctgagcactggaccaaggagcattgtggggctgtgtgcttcgtgaaggataacccccagaagtcctacttcatccgcctttacggccttcaggctggtcggctgctctgggaacaggagctgtactcacagcttgtctactccacccccacccccttcttccacaccttcgctggagatgactgccaagcggggctgaactttgcagacgaggacgaggcccaggccttccgggccctcgtgcaggagaagatacaaaaaaggaatcagaggcaaagtggagacagacgccagctacccccaccaccaacaccagccaatgaagagagaagaggagggctcccacccctgcccctgcatccaggtggagaccaaggaggccctccagtgggtccgctctccctggggctggcgacagtggacatccagaaccctgacatcacgagttcacgataccgtgggctcccagcacctggacctagcccagctgataagaaacgctcagggaagaagaagatcagcaaagctgatattggtgcacccagtggattcaagcatgtcagccacgtggggtgggacccccagaatggatttgacgtgaacaacctcgacccagatctgcggagtctgttctccagggcaggaatcagcgaggcccagctcaccgacgccgagacctctaaacttatctacgacttcattgaggaccagggtgggctggaggctgtgcggcaggagatgaggcgccaggagccacttccgccgcccccaccgccatctcgaggagggaaccagctcccccggccccctattgtggggggtaacaagggtcgttctggtccactgccccctgtacctttggggattgccccacccccaccaacaccccggggacccccacccccaggccgagggggccctccaccaccaccccctccagctactggacgttctggaccactgccccctccaccccctggagctggtgggccacccatgccaccaccaccgccaccaccgccaccgccgcccagctccgggaatggaccagcccctcccccactccctcctgctctggtgcctgccgggggcctggcccctggtgggggtcggggagcgcttttggatcaaatccggcagggaattcagctgaacaagacccctggggccccagagagctcagcgctgcagccaccacctcagagctcagagggactggtgggggccctgatgcacgtgatgcagaagagaagcagagccatccactcctccgacgaaggggaggaccaggctggcgatgaagatgaagatgatgaatgggatgac

SEQ ID NO: 75 - DNA, 1506 bp, Artificial Sequence
  Synthetic
atgtctggcggacctatgggaggtagacctggtggaagaggtgctcctgccgtgcagcagaacatcccttctacactgctgcaggaccacgagaaccagcggctgtttgagatgctgggcagaaagtgtctgaccctggctacagctgtggtgcagctgtatctggcacttcctccaggcgccgagcactggaccaaagaacattgtggcgccgtgtgcttcgtgaaggacaaccctcagaagtcctacttcatccggctgtacggactgcaggctggcagactgctgtgggagcaagagctgtactcccagctggtgtacagcacccctacacctttcttccacacctttgccggcgacgattgtcaggccggactgaactttgccgacgaggatgaagcccaggccttcagagcactggtgcaagagaagatccagaagcggaaccagagacagagcggcgacagaaggcaactgcctcctccacctacaccagccaacgaggaaagaagaggcggactgcctccactgcctcttcatcctggcggagatcaaggtggacctcctgtgggaccactgtctcttggactggccaccgtggacattcagaaccccgatatcaccagcagccggtacagaggacttcccgctcctggaccatctcctgccgacaagaagagatccgggaagaagaagatcagcaaggccgacatcggagcccctagcggctttaaacacgtgtcccacgttggatgggacccacagaacggcttcgacgtgaacaatctggaccccgacctgcggagcctgttttctagagccggaatctctgaggcccagctgaccgatgccgagacaagcaagctgatctacgacttcatcgaggaccaaggcggcctggaagccgtgcgacaagagatgagaaggcaagagcctctgccaccacctccacctccatctagaggcggaaaccagctgcctagacctcctatcgttggcggcaacaagggaagatctggccctctgcctcctgtgcctctgggaattgctccaccaccaccaacacctagaggcccgcctccaccaggcagaggtggtcctccgccgccacctcctccagcaacaggcagatctggaccacttcctcctccaccacctggtgctggtggacctccaatgccaccgccaccgcctccgccacctccgcctccaagttctggaaatggacctgctcctcctcctttgcctcctgctttggttcctgctggcggattggctccaggcggaggaagaggcgcactcctggatcagatcagacagggcatccagctgaacaagacccctggcgctcctgagagttctgctctgcaaccgccaccacagtctagcgaaggacttgtgggagccctgatgcacgtgatgcagaagagaagcagagccatccacagcagcgacgaaggcgaagatcaagctggcgacgaagatgaggacgacgagtgggacgat

SEQ ID NO: 76 - protein, 502 aa, Artificial Sequence
  Synthetic
MSGGPMGGRPGGRGAPAVQQNIPSTLLQDHENQRLFEMLGRKCLTLATAVVQLYLALPPGAEHWTKEHCGAVCFVKDNPQKSYFIRLYGLQAGRLLWEQELYSQLVYSTPTPFFHTFAGDDCQAGLNFADEDEAQAFRALVQEKIQKRNQRQSGDRRQLPPPPTPANEERRGGLPPLPLHPGGDQGGPPVGPLSLGLATVDIQNPDITSSRYRGLPAPGPSPADKKRSGKKKISKADIGAPSGFKHVSHVGWDPQNGFDVNNLDPDLRSLFSRAGISEAQLTDAETSKLIYDFIEDQGGLEAVRQEMRRQEPLPPPPPPSRGGNQLPRPPIVGGNKGRSGPLPPVPLGIAPPPPTPRGPPPPGRGGPPPPPPPATGRSGPLPPPPPGAGGPPMPPPPPPPPPPPSSGNGPAPPPLPPALVPAGGLAPGGGRGALLDQIRQGIQLNKTPGAPESSALQPPPQSSEGLVGALMHVMQKRSRAIHSSDEGEDQAGDEDEDDEWDD

SEQ ID NO: 77 - DNA, 590 bp, Artificial Sequence
  Synthetic
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgca

SEQ ID NO: 78 - DNA, 590 bp, Artificial Sequence
  Synthetic
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgttcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgca

SEQ ID NO: 79 - DNA, 288 bp, Artificial Sequence
  Synthetic
tttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccagtccctatcagtgatagagaaaagtgaaagtcgagtttaccactccctatcagtgatagagaaaagtgaaagtcgagtttaccactccctatcagtgatagagaaaagtgaaa

SEQ ID NO: 80 - DNA, 449 bp, Artificial Sequence
  Synthetic
gatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatc

SEQ ID NO: 81 - DNA, 9345 bp, Artificial Sequence
  Synthetic
ggccgcctcggccaaacagcccttgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccagtccctatcagtgatagagaaaagtgaaagtcgagtttaccactccctatcagtgatagagaaaagtgaaagtcgagtttaccactccctatcagtgatagagaaaagtgaaagtcgagctcgccatgggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatactggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcaggatcttcagacctggaaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccataaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggaccgagctcaagcttcgaagcgatcgcacgcgtggatccgaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcggcgcgccaattcaagcgagaagacaagggcagccgccaccatgagtgggggcccaatgggaggaaggcccgggggccgaggagcaccagcggttcagcagaacataccctccaccctcctccaggaccacgagaaccagcgactctttgagatgcttggacgaaaatgcttgacgctggccactgcagttgttcagctgtacctggcgctgccccctggagctgagcactggaccaaggagcattgtggggctgtgtgcttcgtgaaggataacccccagaagtcctacttcatccgcctttacggccttcaggctggtcggctgctctgggaacaggagctgtactcacagcttgtctactccacccccacccccttcttccacaccttcgctggagatgactgccaagcggggctgaactttgcagacgaggacgaggcccaggccttccgggcactcgtgcaggagaagatacaaaaaaggaatcagaggcaaagtggagacagacgccagctacccccaccaccaacaccagccaatgaagagagaagaggagggctcccacccctgcccctgcatccaggtggagaccaaggaggccctccagtgggtccgctctccctggggctggcgacagtggacatccagaaccctgacatcacgagttcacgataccgtgggctcccagcacctggacctagcccagctgataagaaacgctcagggaagaagaagatcagcaaagctgatattggtgcacccagtggattcaagcatgtcagccacgtggggtgggacccccagaatggatttgacgtgaacaacctcgacccagatctgcggagtctgttctccagggcaggaatcagcgaggcccagctcaccgacgccgagacctctaaacttatctacgacttcattgaggaccagggtgggctggaggctgtgcggcaggagatgaggcgccaggagccacttccgccgcccccaccgccatctcgaggagggaaccagctcccccggccccctattgtggggggtaacaagggtcgttctggtccactgccccctgtacctttggggattgccccacccccaccaacaccccggggacccccacccccaggccgagggggtcctccaccaccaccccctccagctactggacgttctggaccactgccccctccaccccctggagctggtgggccacccatgccaccaccaccgccaccaccgccaccgccgcccagctccgggaatggaccagcccctcccccactccctcctgctctggtgcctgccgggggcctggcccctggtgggggtcggggagcgcttttggatcaaatccggcagggaattcagctgaacaagacccctggggccccagagagctcagcgctgcagccaccacctcagagctcagagggactggtgggggccctgatgcacgtgatgcagaagagaagcagagccatccactcctccgacgaaggggaggaccaggctggcgatgaagatgaagatgatgaatgggatgactgataactagtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcacgtacgaccggtgcggccgcatcgatgccgtagtacctttaagaccaatgacttacaaggcagctgtagatcttagccactttttaaaagaaaaggggggactggaagggctaattcactcccaaagaagacaagatagccccatcctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatccagacaccatcaaacatgcaggctcagacacatgatatcaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctccccagatctgctttttgcctgtactgggtctctctggttagaccagatctgagcctgggagctctctggctaactagggaacccactgcttaagcctcaataaagcttcagctgctcgagctagcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattc

SEQ ID NO: 82 - DNA, 9345 bp, Artificial Sequence
  Synthetic
ggccgcctcggccaaacagcccttgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccagtccctatcagtgatagagaaaagtgaaagtcgagtttaccactccctatcagtgatagagaaaagtgaaagtcgagtttaccactccctatcagtgatagagaaaagtgaaagtcgagctcgccatgggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatactggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcaggatcttcagacctggaaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccataaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggaccgagctcaagcttcgaagcgatcgcacgcgtggatccgaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcggcgcgccaattcaagcgagaagacaagggcagccgccaccatgagtgggggcccaatgggaggaaggcccgggggccgaggagcaccagcggttcagcagaacataccctccaccctcctccaggaccacgagaaccagcgactctttgagatgcttggacgaaaatgcttgacgctggccactgcagttgttcagctgtacctggcgctgccccctggagctgagcactggaccaaggagcattgtggggctgtgtgcttcgtgaaggataacccccagaagtcctacttcatccgcctttacggccttcaggctggtcggctgctctgggaacaggagctgtactcacagcttgtctactccacccccacccccttcttccacaccttcgctggagatgactgccaagcggggctgaactttgcagacgaggacgaggcccaggccttccgggcactcgtgcaggagaagatacaaaaaaggaatcagaggcaaagtggagacagacgccagctacccccaccaccaacaccagccaatgaagagagaagaggagggctcccacccctgcccctgcatccaggtggagaccaaggaggccctccagtgggtccgctctccctggggctggcgacagtggacatccagaaccctgacatcacgagttcacgataccgtgggctcccagcacctggacctagcccagctgataagaaacgctcagggaagaagaagatcagcaaagctgatattggtgcacccagtggattcaagcatgtcagccacgtggggtgggacccccagaatggatttgacgtgaacaacctcgacccagatctgcggagtctgttctccagggcaggaatcagcgaggcccagctcaccgacgccgagacctctaaacttatctacgacttcattgaggaccagggtgggctggaggctgtgcggcaggagatgaggcgccaggagccacttccgccgcccccaccgccatctcgaggagggaaccagctcccccggccccctattgtggggggtaacaagggtcgttctggtccactgccccctgtacctttggggattgccccacccccaccaacaccccggggacccccacccccaggccgagggggtcctccaccaccaccccctccagctactggacgttctggaccactgccccctccaccccctggagctggtgggccacccatgccaccaccaccgccaccaccgccaccgccgcccagctccgggaatggaccagcccctcccccactccctcctgctctggtgcctgccgggggcctggcccctggtgggggtcggggagcgcttttggatcaaatccggcagggaattcagctgaacaagacccctggggccccagagagctcagcgctgcagccaccacctcagagctcagagggactggtgggggccctgatgcacgtgatgcagaagagaagcagagccatccactcctccgacgaaggggaggaccaggctggcgatgaagatgaagatgatgaatgggatgactgataactagtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcacgtacgaccggtgcggccgcatcgatgccgtagtacctttaagaccaatgacttacaaggcagctgtagatcttagccactttttaaaagaaaaggggggactggaagggctaattcactcccaaagaagacaagatagccccatcctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatcctgacaccatcaaacatgcaggctcagacacatgatatcaagctttttccccgtatcccccctggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctccccagatctgctttttgcctgtactgggtctctctggttagaccagatctgagcctgggagctctctggctaactagggaacccactgcttaagcctcaataaagcttcagctgctcgagctagcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattc

SEQ ID NO: 83 - DNA, 9345 bp, Artificial Sequence
  Synthetic
ggccgcctcggccaaacagcccttgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccagtccctatcagtgatagagaaaagtgaaagtcgagtttaccactccctatcagtgatagagaaaagtgaaagtcgagtttaccactccctatcagtgatagagaaaagtgaaagtcgagctcgccatgggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatactggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcaggatcttcagacctggaaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccataaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggaccgagctcaagcttcgaagcgatcgcacgcgtggatccgaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcggcgcgccaattcaagcgagaagacaagggcagccgccaccatgagtgggggcccaatgggaggaaggcccgggggccgaggagcaccagcggttcagcagaacataccctccaccctcctccaggaccacgagaaccagcgactctttgagatgcttggacgaaaatgcttgacgctggccactgcagttgttcagctgtacctggcgctgccccctggagctgagcactggaccaaggagcattgtggggctgtgtgcttcgtgaaggataacccccagaagtcctacttcatccgcctttacggccttcaggctggtcggctgctctgggaacaggagctgtactcacagcttgtctactccacccccacccccttcttccacaccttcgctggagatgactgccaagcggggctgaactttgcagacgaggacgaggcccaggccttccgggcactcgtgcaggagaagatacaaaaaaggaatcagaggcaaagtggagacagacgccagctacccccaccaccaacaccagccaatgaagagagaagaggagggctcccacccctgcccctgcatccaggtggagaccaaggaggccctccagtgggtccgctctccctggggctggcgacagtggacatccagaaccctgacatcacgagttcacgataccgtgggctcccagcacctggacctagcccagctgataagaaacgctcagggaagaagaagatcagcaaagctgatattggtgcacccagtggattcaagcatgtcagccacgtggggtgggacccccagaatggatttgacgtgaacaacctcgacccagatctgcggagtctgttctccagggcaggaatcagcgaggcccagctcaccgacgccgagacctctaaacttatctacgacttcattgaggaccagggtgggctggaggctgtgcggcaggagatgaggcgccaggagccacttccgccgcccccaccgccatctcgaggagggaaccagctcccccggccccctattgtggggggtaacaagggtcgttctggtccactgccccctgtacctttggggattgccccacccccaccaacaccccggggacccccacccccaggccgagggggtcctccaccaccaccccctccagctactggacgttctggaccactgccccctccaccccctggagctggtgggccacccatgccaccaccaccgccaccaccgccaccgccgcccagctccgggaatggaccagcccctcccccactccctcctgctctggtgcctgccgggggcctggcccctggtgggggtcggggagcgcttttggatcaaatccggcagggaattcagctgaacaagacccctggggccccagagagctcagcgctgcagccaccacctcagagctcagagggactggtgggggccctgatgcacgtgatgcagaagagaagcagagccatccactcctccgacgaaggggaggaccaggctggcgatgaagatgaagatgatgaatgggatgactgataactagtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcacgtacgaccggtgcggccgcatcgatgccgtagtacctttaagaccaatgacttacaaggcagctgtagatcttagccactttttaaaagaaaaggggggactggaagggctaattcactcccaaagaagacaagatggggagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagcttgatatcatgtgtctgagcctgcatgtttgatggtgtctggatgcaagcagaaggggtggaagagcttgcctggagagatacagctgggtcagtaggactgggacaggcagctggagaattgccatgtagatgttcatacaatcgtcaaatcatgaaggctggaaaagccctccaagatccccaagaccaaccccaacccacccaccgtgcccactggccatgtccctcagtgccacatccccacagttcttcatcacctccagggacggtgacccccccacctccgtgggcagctgtgccactgcagcaccgctctttggagaaggtaaatcttgctaaatccagcccgaccctcccctggcacaacgtaaggccattatctctcatccaactccaggacggagtcagtgaggatggggctagatctgctttttgcctgtactgggtctctctggttagaccagatctgagcctgggagctctctggctaactagggaacccactgcttaagcctcaataaagcttcagctgctcgagctagcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattc

SEQ ID NO: 84 - DNA, 9345 bp, Artificial Sequence
  Synthetic
ggccgcctcggccaaacagcccttgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccagtccctatcagtgatagagaaaagtgaaagtcgagtttaccactccctatcagtgatagagaaaagtgaaagtcgagtttaccactccctatcagtgatagagaaaagtgaaagtcgagctcgccatgggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatactggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcaggatcttcagacctggaaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccataaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggaccgagctcaagcttcgaagcgatcgcacgcgtggatccgaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcggcgcgccaattcaagcgagaagacaagggcagccgccaccatgagtgggggcccaatgggaggaaggcccgggggccgaggagcaccagcggttcagcagaacataccctccaccctcctccaggaccacgagaaccagcgactctttgagatgcttggacgaaaatgcttgacgctggccactgcagttgttcagctgtacctggcgctgccccctggagctgagcactggaccaaggagcattgtggggctgtgtgcttcgtgaaggataacccccagaagtcctacttcatccgcctttacggccttcaggctggtcggctgctctgggaacaggagctgtactcacagcttgtctactccacccccacccccttcttccacaccttcgctggagatgactgccaagcggggctgaactttgcagacgaggacgaggcccaggccttccgggcactcgtgcaggagaagatacaaaaaaggaatcagaggcaaagtggagacagacgccagctacccccaccaccaacaccagccaatgaagagagaagaggagggctcccacccctgcccctgcatccaggtggagaccaaggaggccctccagtgggtccgctctccctggggctggcgacagtggacatccagaaccctgacatcacgagttcacgataccgtgggctcccagcacctggacctagcccagctgataagaaacgctcagggaagaagaagatcagcaaagctgatattggtgcacccagtggattcaagcatgtcagccacgtggggtgggacccccagaatggatttgacgtgaacaacctcgacccagatctgcggagtctgttctccagggcaggaatcagcgaggcccagctcaccgacgccgagacctctaaacttatctacgacttcattgaggaccagggtgggctggaggctgtgcggcaggagatgaggcgccaggagccacttccgccgcccccaccgccatctcgaggagggaaccagctcccccggccccctattgtggggggtaacaagggtcgttctggtccactgccccctgtacctttggggattgccccacccccaccaacaccccggggacccccacccccaggccgagggggtcctccaccaccaccccctccagctactggacgttctggaccactgccccctccaccccctggagctggtgggccacccatgccaccaccaccgccaccaccgccaccgccgcccagctccgggaatggaccagcccctcccccactccctcctgctctggtgcctgccgggggcctggcccctggtgggggtcggggagcgcttttggatcaaatccggcagggaattcagctgaacaagacccctggggccccagagagctcagcgctgcagccaccacctcagagctcagagggactggtgggggccctgatgcacgtgatgcagaagagaagcagagccatccactcctccgacgaaggggaggaccaggctggcgatgaagatgaagatgatgaatgggatgactgataactagtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcacgtacgaccggtgcggccgcatcgatgccgtagtacctttaagaccaatgacttacaaggcagctgtagatcttagccactttttaaaagaaaaggggggactggaagggctaattcactcccaaagaagacaagatagccccatcctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatcctgacaccatcaaacatgcaggctcagacacatgatatcaagctttttccccgtatcccccctggtgtctgctggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctccccagatctgctttttgcctgtactgggtctctctggttagaccagatctgagcctgggagctctctggctaactagggaacccactgcttaagcctcaataaagcttcagctgctcgagctagcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattc

SEQ ID NO: 85 - DNA, 2141 bp, Artificial Sequence
  Synthetic
gtaaatacacttgcaaaggaggatgtttttagtagcaatttgtactgatggtatggggccaagagatatatcttagagggagggctgagggtttgaagtccaactcctaagccagtgccagaagagccaaggacaggtacggctgtcatcacttagacctcaccctgtggagccacaccctagggttggccaatctactcccaggagcagggagggcaggagccagggctgggcataaaagtcagggcagagccatctattgcttacatttgcttctgacacaactgtgttcactagcaacctcaaacagacaccatgggtcatttcacagaggaggacaaggctactatcacaagcctgtgggacaaggtgaatgtggaagatgctggaggagaaaccctgggaaggtaggctctggtgaccaggacaagggagggaaggaaggaccctgtgcctggcaaaagtccaggttgcttctcaggatttgtggcaccttctgactgtcaaactgttcttgtcaatctcacaggctcctggttgtctacccatggacccagaggttctttgacagctttggcaacctgtcctctgcctctgccatcatgggcaaccccaaagtcaaggcacatggcaagaaggtgctgacttccttgggagatgccataaagcacctggatgatctcaagggcacctttgcccagctgagtgaactgcactgtgacaagctgcatgtggatcctgagaacttcaaggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtcaccgtgctggccattcactttggcaaagaattcacccctgaggtgcaggcttcctggcagaagatggtgactgcagtggccagtgccctgtcctccagataccactgagcctcttgcccatgattcagagctttcaaggataggctttattctgcaagcaatacaaataataaatctattctgctgagagatcacacatgattttcttcagctcttttttttacatctttttaaatatatgagccacaaagggtttatattgagggaagtgtgtatgtgtatttctgcatgcctgtttgtgtttgtggtgtgtgcatgctcctcatttatttttatatgagatgtgcattttgttgagcaaataaaagcagtaaagacacttgtacacgggagttctgcaagtgggagtaaatggtgtaggagaaatccggtgggaagaaagacctctataggacaggacttctcagaaacagatgttttggaagagatgggaaaaggttcagtgaagacctgggggctggattgattgcagctgagtagcaaggatggttcttaatgaagggaaagtgttccaagctcggctagccggtgctagtctcccggaactatcactctttcacagtctgctttggaaggactgggcttagtatgaaaagttaggactgagaagaatttgaaagggggctttttgtagcttgatattcactactgtcttattaccctatcataggcccaccccaaatggaagtcccattcttcctcaggatgtttaagattagcattcaggaagagatcagaggtctgctggctcccttatcatgtcccttatggtgcttctggctccggctagcaccggtgatgatcctcgcgagctcgactctagaggatcccc

SEQ ID NO: 86 - DNA, 2141 bp, Artificial Sequence
  Synthetic
ggggatcctctagagtcgagctcgcgaggatcatcaccggtgctagccggagccagaagcaccataagggacatgataagggagccagcagacctctgatctcttcctgaatgctaatcttaaacatcctgaggaagaatgggacttccatttggggtgggcctatgatagggtaataagacagtagtgaatatcaagctacaaaaagccccctttcaaattcttctcagtcctaacttttcatactaagcccagtccttccaaagcagactgtgaaagagtgatagttccgggagactagcaccggctagccgagcttggaacactttcccttcattaagaaccatccttgctactcagctgcaatcaatccagcccccaggtcttcactgaaccttttcccatctcttccaaaacatctgtttctgagaagtcctgtcctatagaggtctttcttcccaccggatttctcctacaccatttactcccacttgcagaactcccgtgtacaagtgtctttactgcttttatttgctcaacaaaatgcacatctcatataaaaataaatgaggagcatgcacacaccacaaacacaaacaggcatgcagaaatacacatacacacttccctcaatataaaccctttgtggctcatatatttaaaaagatgtaaaaaaaagagctgaagaaaatcatgtgtgatctctcagcagaatagatttattatttgtattgcttgcagaataaagcctatccttgaaagctctgaatcatgggcaagaggctcagtggtatctggaggacagggcactggccactgcagtcaccatcttctgccaggaagcctgcacctcaggggtgaattctttgccaaagtgaatggccagcacggtgaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagaaatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtaccctgttacttctccccttcctatgacatgaacttaaccatagaaaagaaggggaaagaaaacatcaagggtcccatagactcaccttgaagttctcaggatccacatgcagcttgtcacagtgcagttcactcagctgggcaaaggtgcccttgagatcatccaggtgctttatggcatctcccaaggaagtcagcaccttcttgccatgtgccttgactttggggttgcccatgatggcagaggcagaggacaggttgccaaagctgtcaaagaacctctgggtccatgggtagacaaccaggagcctgtgagattgacaagaacagtttgacagtcagaaggtgccacaaatcctgagaagcaacctggacttttgccaggcacagggtccttccttccctcccttgtcctggtcaccagagcctaccttcccagggtttctcctccagcatcttccacattcaccttgtcccacaggcttgtgatagtagccttgtcctcctctgtgaaatgacccatggtgtctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttac

SEQ ID NO: 87 - DNA, 476 bp, Artificial Sequence
  Synthetic
gtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 88 - DNA, 476 bp, Artificial Sequence
  Synthetic
ctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagaaatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtaccctgttacttctccccttcctatgacatgaacttaaccatagaaaagaaggggaaagaaaacatcaagggtcccatagactcac

SEQ ID NO: 89 - DNA, 412 bp, Artificial Sequence
  Synthetic
ggggagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagctttaggctgaaagagagatttagaatgacagaatcatagaacggcctgggttgcaaaggagcacagtgctcatccagatccaaccccctgctatgtgcagggtcatcaaccagcagcccaggctgcccagagccacatccagcctggccttgaatgcctgcagggat

SEQ ID NO: 90 - DNA, 412 bp, Artificial Sequence
  Synthetic
atccctgcaggcattcaaggccaggctggatgtggctctgggcagcctgggctgctggttgatgaccctgcacatagcagggggttggatctggatgagcactgtgctcctttgcaacccaggccgttctatgattctgtcattctaaatctctctttcagcctaaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctcccc

SEQ ID NO: 91 - DNA, 2141 bp, Artificial Sequence
  Synthetic
ggggatcctctagagtcgagctcgcgaggatcatcaccggtgctagccggagccagaagcaccataagggacatgataagggagccagcagacctctgatctcttcctgaatgctaatcttaaacatcctgaggaagaatgggacttccatttggggtgggcctatgatagggtaataagacagtagtgaatatcaagctacaaaaagccccctttcaaattcttctcagtcctaacttttcatactaagcccagtccttccaaagcagactgtgaaagagtgatagttccgggagactagcaccggctagccgagcttggaacactttcccttcattaagaaccatccttgctactcagctgcaatcaatccagcccccaggtcttcactgaaccttttcccatctcttccaaaacatctgtttctgagaagtcctgtcctatagaggtctttcttcccaccggatttctcctacaccatttactcccacttgcagaactcccgtgtacaagtgtctttactgcttttatttgctcaacaaaatgcacatctcatataaaaataaatgaggagcatgcacacaccacaaacacaaacaggcatgcagaaatacacatacacacttccctcaatataaaccctttgtggctcatatatttaaaaagatgtaaaaaaaagagctgaagaaaatcatgtgtgatctctcagcagaatagatttattatttgtattgcttgcagaataaagcctatccttgaaagctctgaatcatgggcaagaggctcagtggtatctggaggacagggcactggccactgcagtcaccatcttctgccaggaagcctgcacctcaggggtgaattctttgccaaagtgaatggccagcacggtgaccagcacgttgcccaggagctgtgggaggaagataagagatatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagaaatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtaccctgttacttctccccttcctatgacatgaacttaaccatagaaaagaaggggaaagaaaacatcaagggtcccatagactcaccttgaagttctcaggatccacatgcagcttgtcacagtgcagttcactcagctgggcaaaggtgcccttgagatcatccaggtgctttatggcatctcccaaggaagtcagcaccttcttgccatgtgccttgactttggggttgcccatgatggcagaggcagaggacaggttgccaaagctgtcaaagaacctctgggtccatgggtagacaaccaggagcctgtgagattgacaagaacagtttgacagtcagaaggtgccacaaatcctgagaagcaacctggacttttgccaggcacagggtccttccttccctcccttgtcctggtcaccagagcctaccttcccagggtttctcctccagcatcttccacattcaccttgtcccacaggcttgtgatagtagccttgtcctcctctgtgaaatgacccatggtgtctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttac

SEQ ID NO: 92 - DNA, 476 bp, Artificial Sequence
  Synthetic
ctgtgggaggaagataagagatatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagaaatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtaccctgttacttctccccttcctatgacatgaacttaaccatagaaaagaaggggaaagaaaacatcaagggtcccatagactcac

SEQ ID NO: 93 - DNA, 412 bp, Artificial Sequence
  Synthetic
atccctgcaggcattcaaggccaggctggatgtggctctgggcagcctgggctgctggttgatgaccctgcacatagcagggggttggatctggatgagcactgtgctcctttgcaacccaggccgttctatgattctgtcattctaaatctctctttcagcctaaagctttttccccgtatcccccctggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctcccc

SEQ ID NO: 94 - DNA, 412 bp, Artificial Sequence
  Synthetic
atccctgcaggcattcaaggccaggctggatgtggctctgggcagcctgggctgctggttgatgaccctgcacatagcagggggttggatctggatgagcactgtgctcctttgcaacccaggccgttctatgattctgtcattctaaatctctctttcagcctaaagctttttccccgtatcccccctggtgtctgctggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctcccc

SEQ ID NO: 95 - DNA, 412 bp, Artificial Sequence
  Synthetic
atccctgcaggcattcaaggccaggctggatgtggctctgggcagcctgggctgctggttgatgaccctgcacatagcagggggttggatctggatgagcactgtgctcctttgcaacccaggccgttctatgattctgtcattctaaatctctctttcagcctaaagctttttccccgtatccccccaggtgtctgctggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctcccc

SEQ ID NO: 96 - DNA, 20 bp, Artificial Sequence
  Synthetic
aagataagaggtatgaacat

SEQ ID NO: 97 - DNA, 20 bp, Artificial Sequence
  Synthetic
aagataagagatatgaacat

SEQ ID NO: 98 - DNA, 93 bp, Artificial Sequence
  Synthetic
ccttcccagggtttctcctccagcatcttccacattcaccttgtcccacaggcttgtgatagtagccttgtcctcctctgtgaaatgacccat

SEQ ID NO: 99 - DNA, 223 bp, Artificial Sequence
  Synthetic
cttgaagttctcaggatccacatgcagcttgtcacagtgcagttcactcagctgggcaaaggtgcccttgagatcatccaggtgctttatggcatctcccaaggaagtcagcaccttcttgccatgtgccttgactttggggttgcccatgatggcagaggcagaggacaggttgccaaagctgtcaaagaacctctgggtccatgggtagacaaccaggagc

SEQ ID NO: 100 - DNA, 216 bp, Artificial Sequence
  Synthetic
gtgatctctcagcagaatagatttattatttgtattgcttgcagaataaagcctatccttgaaagctctgaatcatgggcaagaggctcagtggtatctggaggacagggcactggccactgcagtcaccatcttctgccaggaagcctgcacctcaggggtgaattctttgccaaagtgaatggccagcacggtgaccagcacgttgcccaggag

SEQ ID NO: 101 - DNA, 539 bp, Artificial Sequence
  Synthetic
cagacaccatgggtcatttcacagaggaggacaaggctactatcacaagcctgtgggacaaggtgaatgtggaagatgctggaggagaaaccctgggaaggctcctggttgtctacccatggacccagaggttctttgacagctttggcaacctgtcctctgcctctgccatcatgggcaaccccaaagtcaaggcacatggcaagaaggtgctgacttccttgggagatgccataaagcacctggatgatctcaagggcacctttgcccagctgagtgaactgcactgtgacaagctgcatgtggatcctgagaacttcaagctcctgggcaacgtgctggtcaccgtgctggccattcactttggcaaagaattcacccctgaggtgcaggcttcctggcagaagatggtgactgcagtggccagtgccctgtcctccagataccactgagcctcttgcccatgattcagagctttcaaggataggctttattctgcaagcaatacaaataataaatctattctgctgagagatcac

SEQ ID NO: 102 - DNA, 444 bp, Artificial Sequence
  Synthetic
atgggtcatttcacagaggaggacaaggctactatcacaagcctgtgggacaaggtgaatgtggaagatgctggaggagaaaccctgggaaggctcctggttgtctacccatggacccagaggttctttgacagctttggcaacctgtcctctgcctctgccatcatgggcaaccccaaagtcaaggcacatggcaagaaggtgctgacttccttgggagatgccataaagcacctggatgatctcaagggcacctttgcccagctgagtgaactgcactgtgacaagctgcatgtggatcctgagaacttcaagctcctgggcaacgtgctggtcaccgtgctggccattcactttggcaaagaattcacccctgaggtgcaggcttcctggcagaagatggtgactgcagtggccagtgccctgtcctccagataccactga

SEQ ID NO: 103 - protein, 147 aa, Artificial Sequence
  Synthetic
MGHFTEEDKATITSLWDKVNVEDAGGETLGRLLVVYPWTQRFFDSFGNLSSASAIMGNPKVKAHGKKVLTSLGDAIKHLDDLKGTFAQLSELHCDKLHVDPENFKLLGNVLVTVLAIHFGKEFTPEVQASWQKMVTAVASALSSRYH

SEQ ID NO: 104 - DNA, 449 bp, Artificial Sequence; identical to SEQ ID NO: 80
  Synthetic

SEQ ID NO: 105 - DNA, 3055 bp, Artificial Sequence
  Synthetic
gtatgtgagcatgtgtcctctaacagcacaggccttttgccacctagctgtccaggggtgccttaaaatggcaaacaaggtttgttttcttttcctgttttcatgccttcctcttccatatccttgtttcatattaatacatgtgtatagatcctaaaaatctatacacatgtattaataaagcctgattctgccgcttctaggtatagaggccacctgcaagataaatatttgattcacaataactaatcattctatggcaattgataacaacaaatatatatatatatatatatatacgtatatgtgtatatatatatatatattcaggaaataatatattctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaagggaattccggtgccctgcttaggagcttaatctttaatgaaagctaagctttcattaaaaaaagtctaaccagctgcattcgactttgactgcagcagctggttagaaggttctactggaggagggtcccagcccattgctaaattaacatcaggctctgagactggcagtatatctctaacagtggttgatgctatcttctggaacttgcctgctacattgagaccactgacccatacataggaagcccatagctctgtcctgaactgttaggccactggtccagagagtgtgcatctcctttgatcctcataataaccctatgagatagacacaattattactcttactttatagatgatgatcctgaaaacataggagtcaaggcacttgcccctagctgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcctgctcccaaatttacagtcatagacttcttcatggctgtctcctttatccacagaatgattcctttgcttcattgccccatccatctgatcctcctcatcagtgcagcacagggcccatgagcagtagctgcagagtctcacataggtctggcactgcctctgacatgtccgaccttaggcaaatgcttgactcttctgagctcggatcccttgagctcaggaggtcaaggctgcagtgagacatgatcttgccactgcactccagcctggacagcagagtgaaaccttgcctcacgaaacagaatacaaaaacaaacaaacaaaaaactgctccgcaatgcgcttccttgatgctctaccacataggtctgggtactttgtacacattatctcattgctgttcataattgttagattaattttgtaatattgatattattcctagaaagctgaggcctcaagatgataacttttattttctggacttgtaatagctttctcttgtattcaccatgttgtaactttcttagagtagtaacaatataaagttattgtgagtttttgcaaacacagcaaacacaacgacccatatagacattgatgtgaaattgtctattgtcaatttatgggaaaacaagtatgtactttttctactaagccattgaaacaggaataacagaacaagattgaaagaatacattttccgaaattacttgagtattatacaaagacaagcacgtggacctgggaggagggttattgtccatgactggtgtgtggagacaaatgcaggtttataatagatgggatggcatctagcgcaatgactttgccatcacttttagagagctcttgggggccccagtacacaagaggggacgcagggtatatgtagacatctcattctttttcttagtgtgagaataagaatagccatgacctgagtttatagacaatgagcccttttctctctcccactcagcagctatgagatggcttgccctgcctctctactaggctgactcactccaaggcccagcaatgggcagggctctgtcagggctttgatagcactatctgcagagccagggccgagaaggggtggactccagagactctccctcccattcccgagcagggtttgcttatttatgcatttaaatgatatatttattttaaaagaaataacaggagactgcccagccctggctgtgacatggaaactatgtagaatattttgggttccatttttttttccttctttcagttagaggaaaaggggctcactgcacatacactagacagaaagtcaggagctttgaatccaagcctgatcatttccatgtcatactgagaaagtccccacccttctctgagcctcagtttctctttttataagtaggagtctggagtaaatgatttccaatggctctcatttcaatacaaaatttccgtttattaaatgcatgagcttccgttactccaagactgagaaggaaattgaacctgagactcattgactggcaagatgtccccagaggctctcattcagcaataaaattctcaccttcacccaggcccactagtgtcagatttgcatgc

SEQ ID NO: 106 - DNA, 769 bp, Artificial Sequence
  Synthetic
aggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggaccg

SEQ ID NO: 107 - DNA, 12084 bp, Artificial Sequence
  Synthetic
ggccgcctcggccaaacagcccttgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccagtccctatcagtgatagagaaaagtgaaagtcgagtttaccactccctatcagtgatagagaaaagtgaaagtcgagtttaccactccctatcagtgatagagaaaagtgaaagtcgagctcgccatgggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatactggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcaggatcttcagacctggaaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccataaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggaccgagctcaagcttcgaacgcgtggggatcctctagagtcgagctcgcgaggatcatcaccggtgctagccggagccagaagcaccataagggacatgataagggagccagcagacctctgatctcttcctgaatgctaatcttaaacatcctgaggaagaatgggacttccatttggggtgggcctatgatagggtaataagacagtagtgaatatcaagctacaaaaagccccctttcaaattcttctcagtcctaacttttcatactaagcccagtccttccaaagcagactgtgaaagagtgatagttccgggagactagcaccggctagccgagcttggaacactttcccttcattaagaaccatccttgctactcagctgcaatcaatccagcccccaggtcttcactgaaccttttcccatctcttccaaaacatctgtttctgagaagtcctgtcctatagaggtctttcttcccaccggatttctcctacaccatttactcccacttgcagaactcccgtgtacaagtgtctttactgcttttatttgctcaacaaaatgcacatctcatataaaaataaatgaggagcatgcacacaccacaaacacaaacaggcatgcagaaatacacatacacacttccctcaatataaaccctttgtggctcatatatttaaaaagatgtaaaaaaaagagctgaagaaaatcatgtgtgatctctcagcagaatagatttattatttgtattgcttgcagaataaagcctatccttgaaagctctgaatcatgggcaagaggctcagtggtatctggaggacagggcactggccactgcagtcaccatcttctgccaggaagcctgcacctcaggggtgaattctttgccaaagtgaatggccagcacggtgaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagaaatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtaccctgttacttctccccttcctatgacatgaacttaaccatagaaaagaaggggaaagaaaacatcaagggtcccatagactcaccttgaagttctcaggatccacatgcagcttgtcacagtgcagttcactcagctgggcaaaggtgcccttgagatcatccaggtgctttatggcatctcccaaggaagtcagcaccttcttgccatgtgccttgactttggggttgcccatgatggcagaggcagaggacaggttgccaaagctgtcaaagaacctctgggtccatgggtagacaaccaggagcctgtgagattgacaagaacagtttgacagtcagaaggtgccacaaatcctgagaagcaacctggacttttgccaggcacagggtccttccttccctcccttgtcctggtcaccagagcctaccttcccagggtttctcctccagcatcttccacattcaccttgtcccacaggcttgtgatagtagccttgtcctcctctgtgaaatgacccatggtgtctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttacgacggtatcgatgtatgtgagcatgtgtcctctaacagcacaggccttttgccacctagctgtccaggggtgccttaaaatggcaaacaaggtttgttttcttttcctgttttcatgccttcctcttccatatccttgtttcatattaatacatgtgtatagatcctaaaaatctatacacatgtattaataaagcctgattctgccgcttctaggtatagaggccacctgcaagataaatatttgattcacaataactaatcattctatggcaattgataacaacaaatatatatatatatatatatatacgtatatgtgtatatatatatatatattcaggaaataatatattctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaagggaattccggtgccctgcttaggagcttaatctttaatgaaagctaagctttcattaaaaaaagtctaaccagctgcattcgactttgactgcagcagctggttagaaggttctactggaggagggtcccagcccattgctaaattaacatcaggctctgagactggcagtatatctctaacagtggttgatgctatcttctggaacttgcctgctacattgagaccactgacccatacataggaagcccatagctctgtcctgaactgttaggccactggtccagagagtgtgcatctcctttgatcctcataataaccctatgagatagacacaattattactcttactttatagatgatgatcctgaaaacataggagtcaaggcacttgcccctagctgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcctgctcccaaatttacagtcatagacttcttcatggctgtctcctttatccacagaatgattcctttgcttcattgccccatccatctgatcctcctcatcagtgcagcacagggcccatgagcagtagctgcagagtctcacataggtctggcactgcctctgacatgtccgaccttaggcaaatgcttgactcttctgagctcggatcccttgagctcaggaggtcaaggctgcagtgagacatgatcttgccactgcactccagcctggacagcagagtgaaaccttgcctcacgaaacagaatacaaaaacaaacaaacaaaaaactgctccgcaatgcgcttccttgatgctctaccacataggtctgggtactttgtacacattatctcattgctgttcataattgttagattaattttgtaatattgatattattcctagaaagctgaggcctcaagatgataacttttattttctggacttgtaatagctttctcttgtattcaccatgttgtaactttcttagagtagtaacaatataaagttattgtgagtttttgcaaacacagcaaacacaacgacccatatagacattgatgtgaaattgtctattgtcaatttatgggaaaacaagtatgtactttttctactaagccattgaaacaggaataacagaacaagattgaaagaatacattttccgaaattacttgagtattatacaaagacaagcacgtggacctgggaggagggttattgtccatgactggtgtgtggagacaaatgcaggtttataatagatgggatggcatctagcgcaatgactttgccatcacttttagagagctcttgggggccccagtacacaagaggggacgcagggtatatgtagacatctcattctttttcttagtgtgagaataagaatagccatgacctgagtttatagacaatgagcccttttctctctcccactcagcagctatgagatggcttgccctgcctctctactaggctgactcactccaaggcccagcaatgggcagggctctgtcagggctttgatagcactatctgcagagccagggccgagaaggggtggactccagagactctccctcccattcccgagcagggtttgcttatttatgcatttaaatgatatatttattttaaaagaaataacaggagactgcccagccctggctgtgacatggaaactatgtagaatattttgggttccatttttttttccttctttcagttagaggaaaaggggctcactgcacatacactagacagaaagtcaggagctttgaatccaagcctgatcatttccatgtcatactgagaaagtccccacccttctctgagcctcagtttctctttttataagtaggagtctggagtaaatgatttccaatggctctcatttcaatacaaaatttccgtttattaaatgcatgagcttccgttactccaagactgagaaggaaattgaacctgagactcattgactggcaagatgtccccagaggctctcattcagcaataaaattctcaccttcacccaggcccactagtgtcagatttgcatgcgttcgcgtatcgacgtgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggtacctcaggatatgcccttgactatttgtccgacatagtcaagggcatatccttttttgtgcggccgcatcgatgccgtagtacctttaagaccaatgacttacaaggcagctgtagatcttagccactttttaaaagaaaaggggggactggaagggctaattcactcccaaagaagacaagatccctgcaggcattcaaggccaggctggatgtggctctgggcagcctgggctgctggttgatgaccctgcacatagcagggggttggatctggatgagcactgtgctcctttgcaacccaggccgttctatgattctgtcattctaaatctctctttcagcctaaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctccccagatctgctttttgcctgtactgggtctctctggttagaccagatctgagcctgggagctctctggctaactagggaacccactgcttaagcctcaataaagcttcagctgctcgagctagcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattc

SEQ ID NO: 108 - DNA, 12363 bp, Artificial Sequence
  Synthetic
ggccgcctcggccaaacagcccttgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccagtccctatcagtgatagagaaaagtgaaagtcgagtttaccactccctatcagtgatagagaaaagtgaaagtcgagtttaccactccctatcagtgatagagaaaagtgaaagtcgagctcgccatgggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatactggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcaggatcttcagacctggaaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccataaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggaccgagctcaagcttcgaacgcgtggggatcctctagagtcgagctcgcgaggatcatcaccggtgctagccggagccagaagcaccataagggacatgataagggagccagcagacctctgatctcttcctgaatgctaatcttaaacatcctgaggaagaatgggacttccatttggggtgggcctatgatagggtaataagacagtagtgaatatcaagctacaaaaagccccctttcaaattcttctcagtcctaacttttcatactaagcccagtccttccaaagcagactgtgaaagagtgatagttccgggagactagcaccggctagccgagcttggaacactttcccttcattaagaaccatccttgctactcagctgcaatcaatccagcccccaggtcttcactgaaccttttcccatctcttccaaaacatctgtttctgagaagtcctgtcctatagaggtctttcttcccaccggatttctcctacaccatttactcccacttgcagaactcccgtgtacaagtgtctttactgcttttatttgctcaacaaaatgcacatctcatataaaaataaatgaggagcatgcacacaccacaaacacaaacaggcatgcagaaatacacatacacacttccctcaatataaaccctttgtggctcatatatttaaaaagatgtaaaaaaaagagctgaagaaaatcatgtgtgatctctcagcagaatagatttattatttgtattgcttgcagaataaagcctatccttgaaagctctgaatcatgggcaagaggctcagtggtatctggaggacagggcactggccactgcagtcaccatcttctgccaggaagcctgcacctcaggggtgaattctttgccaaagtgaatggccagcacggtgaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagaaatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtaccctgttacttctccccttcctatgacatgaacttaaccatagaaaagaaggggaaagaaaacatcaagggtcccatagactcaccttgaagttctcaggatccacatgcagcttgtcacagtgcagttcactcagctgggcaaaggtgcccttgagatcatccaggtgctttatggcatctcccaaggaagtcagcaccttcttgccatgtgccttgactttggggttgcccatgatggcagaggcagaggacaggttgccaaagctgtcaaagaacctctgggtccatgggtagacaaccaggagcctgtgagattgacaagaacagtttgacagtcagaaggtgccacaaatcctgagaagcaacctggacttttgccaggcacagggtccttccttccctcccttgtcctggtcaccagagcctaccttcccagggtttctcctccagcatcttccacattcaccttgtcccacaggcttgtgatagtagccttgtcctcctctgtgaaatgacccatggtgtctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttacgacggtatcgatgtatgtgagcatgtgtcctctaacagcacaggccttttgccacctagctgtccaggggtgccttaaaatggcaaacaaggtttgttttcttttcctgttttcatgccttcctcttccatatccttgtttcatattaatacatgtgtatagatcctaaaaatctatacacatgtattaataaagcctgattctgccgcttctaggtatagaggccacctgcaagataaatatttgattcacaataactaatcattctatggcaattgataacaacaaatatatatatatatatatatatacgtatatgtgtatatatatatatatattcaggaaataatatattctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaagggaattccggtgccctgcttaggagcttaatctttaatgaaagctaagctttcattaaaaaaagtctaaccagctgcattcgactttgactgcagcagctggttagaaggttctactggaggagggtcccagcccattgctaaattaacatcaggctctgagactggcagtatatctctaacagtggttgatgctatcttctggaacttgcctgctacattgagaccactgacccatacataggaagcccatagctctgtcctgaactgttaggccactggtccagagagtgtgcatctcctttgatcctcataataaccctatgagatagacacaattattactcttactttatagatgatgatcctgaaaacataggagtcaaggcacttgcccctagctgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcctgctcccaaatttacagtcatagacttcttcatggctgtctcctttatccacagaatgattcctttgcttcattgccccatccatctgatcctcctcatcagtgcagcacagggcccatgagcagtagctgcagagtctcacataggtctggcactgcctctgacatgtccgaccttaggcaaatgcttgactcttctgagctcggatcccttgagctcaggaggtcaaggctgcagtgagacatgatcttgccactgcactccagcctggacagcagagtgaaaccttgcctcacgaaacagaatacaaaaacaaacaaacaaaaaactgctccgcaatgcgcttccttgatgctctaccacataggtctgggtactttgtacacattatctcattgctgttcataattgttagattaattttgtaatattgatattattcctagaaagctgaggcctcaagatgataacttttattttctggacttgtaatagctttctcttgtattcaccatgttgtaactttcttagagtagtaacaatataaagttattgtgagtttttgcaaacacagcaaacacaacgacccatatagacattgatgtgaaattgtctattgtcaatttatgggaaaacaagtatgtactttttctactaagccattgaaacaggaataacagaacaagattgaaagaatacattttccgaaattacttgagtattatacaaagacaagcacgtggacctgggaggagggttattgtccatgactggtgtgtggagacaaatgcaggtttataatagatgggatggcatctagcgcaatgactttgccatcacttttagagagctcttgggggccccagtacacaagaggggacgcagggtatatgtagacatctcattctttttcttagtgtgagaataagaatagccatgacctgagtttatagacaatgagcccttttctctctcccactcagcagctatgagatggcttgccctgcctctctactaggctgactcactccaaggcccagcaatgggcagggctctgtcagggctttgatagcactatctgcagagccagggccgagaaggggtggactccagagactctccctcccattcccgagcagggtttgcttatttatgcatttaaatgatatatttattttaaaagaaataacaggagactgcccagccctggctgtgacatggaaactatgtagaatattttgggttccatttttttttccttctttcagttagaggaaaaggggctcactgcacatacactagacagaaagtcaggagctttgaatccaagcctgatcatttccatgtcatactgagaaagtccccacccttctctgagcctcagtttctctttttataagtaggagtctggagtaaatgatttccaatggctctcatttcaatacaaaatttccgtttattaaatgcatgagcttccgttactccaagactgagaaggaaattgaacctgagactcattgactggcaagatgtccccagaggctctcattcagcaataaaattctcaccttcacccaggcccactagtgtcagatttgcatgcgttcgcgtatcgacgtgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggtacctcaggatatgcccttgactatttgtccgacatagtcaagggcatatccttttttgtgcggccgcatcgatgccgtagtacctttaagaccaatgacttacaaggcagctgtagatcttagccactttttaaaagaaaaggggggactggaagggctaattcactcccaaagaagacaagatagatctgctttttgcctgtactgggtctctctggttagaccagatctgagcctgggagctctctggctaactagggaacccactgcttaagcctcaataaagcttcagctgctcgagctagcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcggccgcctcggccaaacagcccttgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagttatttccccgaaaagtgccacctgacgtctaagaaaccattattatcatgacattacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattc

SEQ ID NO: 109 - DNA, 12363 bp, Artificial Sequence
  Synthetic
ggccgcctcggccaaacagcccttgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccagtccctatcagtgatagagaaaagtgaaagtcgagtttaccactccctatcagtgatagagaaaagtgaaagtcgagtttaccactccctatcagtgatagagaaaagtgaaagtcgagctcgccatgggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatactggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcaggatcttcagacctggaaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccataaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggaccgagctcaagcttcgaacgcgtggggatcctctagagtcgagctcgcgaggatcatcaccggtgctagccggagccagaagcaccataagggacatgataagggagccagcagacctctgatctcttcctgaatgctaatcttaaacatcctgaggaagaatgggacttccatttggggtgggcctatgatagggtaataagacagtagtgaatatcaagctacaaaaagccccctttcaaattcttctcagtcctaacttttcatactaagcccagtccttccaaagcagactgtgaaagagtgatagttccgggagactagcaccggctagccgagcttggaacactttcccttcattaagaaccatccttgctactcagctgcaatcaatccagcccccaggtcttcactgaaccttttcccatctcttccaaaacatctgtttctgagaagtcctgtcctatagaggtctttcttcccaccggatttctcctacaccatttactcccacttgcagaactcccgtgtacaagtgtctttactgcttttatttgctcaacaaaatgcacatctcatataaaaataaatgaggagcatgcacacaccacaaacacaaacaggcatgcagaaatacacatacacacttccctcaatataaaccctttgtggctcatatatttaaaaagatgtaaaaaaaagagctgaagaaaatcatgtgtgatctctcagcagaatagatttattatttgtattgcttgcagaataaagcctatccttgaaagctctgaatcatgggcaagaggctcagtggtatctggaggacagggcactggccactgcagtcaccatcttctgccaggaagcctgcacctcaggggtgaattctttgccaaagtgaatggccagcacggtgaccagcacgttgcccaggagctgtgggaggaagataagagatatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagaaatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtaccctgttacttctccccttcctatgacatgaacttaaccatagaaaagaaggggaaagaaaacatcaagggtcccatagactcaccttgaagttctcaggatccacatgcagcttgtcacagtgcagttcactcagctgggcaaaggtgcccttgagatcatccaggtgctttatggcatctcccaaggaagtcagcaccttcttgccatgtgccttgactttggggttgcccatgatggcagaggcagaggacaggttgccaaagctgtcaaagaacctctgggtccatgggtagacaaccaggagcctgtgagattgacaagaacagtttgacagtcagaaggtgccacaaatcctgagaagcaacctggacttttgccaggcacagggtccttccttccctcccttgtcctggtcaccagagcctaccttcccagggtttctcctccagcatcttccacattcaccttgtcccacaggcttgtgatagtagccttgtcctcctctgtgaaatgacccatggtgtctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttacgacggtatcgatgtatgtgagcatgtgtcctctaacagcacaggccttttgccacctagctgtccaggggtgccttaaaatggcaaacaaggtttgttttcttttcctgttttcatgccttcctcttccatatccttgtttcatattaatacatgtgtatagatcctaaaaatctatacacatgtattaataaagcctgattctgccgcttctaggtatagaggccacctgcaagataaatatttgattcacaataactaatcattctatggcaattgataacaacaaatatatatatatatatatatatacgtatatgtgtatatatatatatatattcaggaaataatatattctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaagggaattccggtgccctgcttaggagcttaatctttaatgaaagctaagctttcattaaaaaaagtctaaccagctgcattcgactttgactgcagcagctggttagaaggttctactggaggagggtcccagcccattgctaaattaacatcaggctctgagactggcagtatatctctaacagtggttgatgctatcttctggaacttgcctgctacattgagaccactgacccatacataggaagcccatagctctgtcctgaactgttaggccactggtccagagagtgtgcatctcctttgatcctcataataaccctatgagatagacacaattattactcttactttatagatgatgatcctgaaaacataggagtcaaggcacttgcccctagctgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcctgctcccaaatttacagtcatagacttcttcatggctgtctcctttatccacagaatgattcctttgcttcattgccccatccatctgatcctcctcatcagtgcagcacagggcccatgagcagtagctgcagagtctcacataggtctggcactgcctctgacatgtccgaccttaggcaaatgcttgactcttctgagctcggatcccttgagctcaggaggtcaaggctgcagtgagacatgatcttgccactgcactccagcctggacagcagagtgaaaccttgcctcacgaaacagaatacaaaaacaaacaaacaaaaaactgctccgcaatgcgcttccttgatgctctaccacataggtctgggtactttgtacacattatctcattgctgttcataattgttagattaattttgtaatattgatattattcctagaaagctgaggcctcaagatgataacttttattttctggacttgtaatagctttctcttgtattcaccatgttgtaactttcttagagtagtaacaatataaagttattgtgagtttttgcaaacacagcaaacacaacgacccatatagacattgatgtgaaattgtctattgtcaatttatgggaaaacaagtatgtactttttctactaagccattgaaacaggaataacagaacaagattgaaagaatacattttccgaaattacttgagtattatacaaagacaagcacgtggacctgggaggagggttattgtccatgactggtgtgtggagacaaatgcaggtttataatagatgggatggcatctagcgcaatgactttgccatcacttttagagagctcttgggggccccagtacacaagaggggacgcagggtatatgtagacatctcattctttttcttagtgtgagaataagaatagccatgacctgagtttatagacaatgagcccttttctctctcccactcagcagctatgagatggcttgccctgcctctctactaggctgactcactccaaggcccagcaatgggcagggctctgtcagggctttgatagcactatctgcagagccagggccgagaaggggtggactccagagactctccctcccattcccgagcagggtttgcttatttatgcatttaaatgatatatttattttaaaagaaataacaggagactgcccagccctggctgtgacatggaaactatgtagaatattttgggttccatttttttttccttctttcagttagaggaaaaggggctcactgcacatacactagacagaaagtcaggagctttgaatccaagcctgatcatttccatgtcatactgagaaagtccccacccttctctgagcctcagtttctctttttataagtaggagtctggagtaaatgatttccaatggctctcatttcaatacaaaatttccgtttattaaatgcatgagcttccgttactccaagactgagaaggaaattgaacctgagactcattgactggcaagatgtccccagaggctctcattcagcaataaaattctcaccttcacccaggcccactagtgtcagatttgcatgcgttcgcgtatcgacgtgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggtacctcaggatatgcccttgactatttgtccgacatagtcaagggcatatccttttttgtgcggccgcatcgatgccgtagtacctttaagaccaatgacttacaaggcagctgtagatcttagccactttttaaaagaaaaggggggactggaagggctaattcactcccaaagaagacaagatagatctgctttttgcctgtactgggtctctctggttagaccagatctgagcctgggagctctctggctaactagggaacccactgcttaagcctcaataaagcttcagctgctcgagctagcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcggccgcctcggccaaacagcccttgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagttatttccccgaaaagtgccacctgacgtctaagaaaccattattatcatgacattacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattc

SEQ ID NO: 110 - DNA, 12084 bp, Artificial Sequence
  Synthetic
ggccgcctcggccaaacagcccttgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccagtccctatcagtgatagagaaaagtgaaagtcgagtttaccactccctatcagtgatagagaaaagtgaaagtcgagtttaccactccctatcagtgatagagaaaagtgaaagtcgagctcgccatgggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatactggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcaggatcttcagacctggaaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccataaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggaccgagctcaagcttcgaacgcgtggggatcctctagagtcgagctcgcgaggatcatcaccggtgctagccggagccagaagcaccataagggacatgataagggagccagcagacctctgatctcttcctgaatgctaatcttaaacatcctgaggaagaatgggacttccatttggggtgggcctatgatagggtaataagacagtagtgaatatcaagctacaaaaagccccctttcaaattcttctcagtcctaacttttcatactaagcccagtccttccaaagcagactgtgaaagagtgatagttccgggagactagcaccggctagccgagcttggaacactttcccttcattaagaaccatccttgctactcagctgcaatcaatccagcccccaggtcttcactgaaccttttcccatctcttccaaaacatctgtttctgagaagtcctgtcctatagaggtctttcttcccaccggatttctcctacaccatttactcccacttgcagaactcccgtgtacaagtgtctttactgcttttatttgctcaacaaaatgcacatctcatataaaaataaatgaggagcatgcacacaccacaaacacaaacaggcatgcagaaatacacatacacacttccctcaatataaaccctttgtggctcatatatttaaaaagatgtaaaaaaaagagctgaagaaaatcatgtgtgatctctcagcagaatagatttattatttgtattgcttgcagaataaagcctatccttgaaagctctgaatcatgggcaagaggctcagtggtatctggaggacagggcactggccactgcagtcaccatcttctgccaggaagcctgcacctcaggggtgaattctttgccaaagtgaatggccagcacggtgaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagaaatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtaccctgttacttctccccttcctatgacatgaacttaaccatagaaaagaaggggaaagaaaacatcaagggtcccatagactcaccttgaagttctcaggatccacatgcagcttgtcacagtgcagttcactcagctgggcaaaggtgcccttgagatcatccaggtgctttatggcatctcccaaggaagtcagcaccttcttgccatgtgccttgactttggggttgcccatgatggcagaggcagaggacaggttgccaaagctgtcaaagaacctctgggtccatgggtagacaaccaggagcctgtgagattgacaagaacagtttgacagtcagaaggtgccacaaatcctgagaagcaacctggacttttgccaggcacagggtccttccttccctcccttgtcctggtcaccagagcctaccttcccagggtttctcctccagcatcttccacattcaccttgtcccacaggcttgtgatagtagccttgtcctcctctgtgaaatgacccatggtgtctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttacgacggtatcgatgtatgtgagcatgtgtcctctaacagcacaggccttttgccacctagctgtccaggggtgccttaaaatggcaaacaaggtttgttttcttttcctgttttcatgccttcctcttccatatccttgtttcatattaatacatgtgtatagatcctaaaaatctatacacatgtattaataaagcctgattctgccgcttctaggtatagaggccacctgcaagataaatatttgattcacaataactaatcattctatggcaattgataacaacaaatatatatatatatatatatatacgtatatgtgtatatatatatatatattcaggaaataatatattctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaagggaattccggtgccctgcttaggagcttaatctttaatgaaagctaagctttcattaaaaaaagtctaaccagctgcattcgactttgactgcagcagctggttagaaggttctactggaggagggtcccagcccattgctaaattaacatcaggctctgagactggcagtatatctctaacagtggttgatgctatcttctggaacttgcctgctacattgagaccactgacccatacataggaagcccatagctctgtcctgaactgttaggccactggtccagagagtgtgcatctcctttgatcctcataataaccctatgagatagacacaattattactcttactttatagatgatgatcctgaaaacataggagtcaaggcacttgcccctagctgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcctgctcccaaatttacagtcatagacttcttcatggctgtctcctttatccacagaatgattcctttgcttcattgccccatccatctgatcctcctcatcagtgcagcacagggcccatgagcagtagctgcagagtctcacataggtctggcactgcctctgacatgtccgaccttaggcaaatgcttgactcttctgagctcggatcccttgagctcaggaggtcaaggctgcagtgagacatgatcttgccactgcactccagcctggacagcagagtgaaaccttgcctcacgaaacagaatacaaaaacaaacaaacaaaaaactgctccgcaatgcgcttccttgatgctctaccacataggtctgggtactttgtacacattatctcattgctgttcataattgttagattaattttgtaatattgatattattcctagaaagctgaggcctcaagatgataacttttattttctggacttgtaatagctttctcttgtattcaccatgttgtaactttcttagagtagtaacaatataaagttattgtgagtttttgcaaacacagcaaacacaacgacccatatagacattgatgtgaaattgtctattgtcaatttatgggaaaacaagtatgtactttttctactaagccattgaaacaggaataacagaacaagattgaaagaatacattttccgaaattacttgagtattatacaaagacaagcacgtggacctgggaggagggttattgtccatgactggtgtgtggagacaaatgcaggtttataatagatgggatggcatctagcgcaatgactttgccatcacttttagagagctcttgggggccccagtacacaagaggggacgcagggtatatgtagacatctcattctttttcttagtgtgagaataagaatagccatgacctgagtttatagacaatgagcccttttctctctcccactcagcagctatgagatggcttgccctgcctctctactaggctgactcactccaaggcccagcaatgggcagggctctgtcagggctttgatagcactatctgcagagccagggccgagaaggggtggactccagagactctccctcccattcccgagcagggtttgcttatttatgcatttaaatgatatatttattttaaaagaaataacaggagactgcccagccctggctgtgacatggaaactatgtagaatattttgggttccatttttttttccttctttcagttagaggaaaaggggctcactgcacatacactagacagaaagtcaggagctttgaatccaagcctgatcatttccatgtcatactgagaaagtccccacccttctctgagcctcagtttctctttttataagtaggagtctggagtaaatgatttccaatggctctcatttcaatacaaaatttccgtttattaaatgcatgagcttccgttactccaagactgagaaggaaattgaacctgagactcattgactggcaagatgtccccagaggctctcattcagcaataaaattctcaccttcacccaggcccactagtgtcagatttgcatgcgttcgcgtatcgacgtgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggtacctcaggatatgcccttgactatttgtccgacatagtcaagggcatatccttttttgtgcggccgcatcgatgccgtagtacctttaagaccaatgacttacaaggcagctgtagatcttagccactttttaaaagaaaaggggggactggaagggctaattcactcccaaagaagacaagatccctgcaggcattcaaggccaggctggatgtggctctgggcagcctgggctgctggttgatgaccctgcacatagcagggggttggatctggatgagcactgtgctcctttgcaacccaggccgttctatgattctgtcattctaaatctctctttcagcctaaagctttttccccgtatcccccctggtgtctgctggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctccccagatctgctttttgcctgtactgggtctctctggttagaccagatctgagcctgggagctctctggctaactagggaacccactgcttaagcctcaataaagcttcagctgctcgagctagcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattc

SEQ ID NO: 111 - DNA, 12084 bp, Artificial Sequence
  Synthetic
ggccgcctcggccaaacagcccttgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccagtccctatcagtgatagagaaaagtgaaagtcgagtttaccactccctatcagtgatagagaaaagtgaaagtcgagtttaccactccctatcagtgatagagaaaagtgaaagtcgagctcgccatgggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatactggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcaggatcttcagacctggaaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccataaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggaccgagctcaagcttcgaacgcgtggggatcctctagagtcgagctcgcgaggatcatcaccggtgctagccggagccagaagcaccataagggacatgataagggagccagcagacctctgatctcttcctgaatgctaatcttaaacatcctgaggaagaatgggacttccatttggggtgggcctatgatagggtaataagacagtagtgaatatcaagctacaaaaagccccctttcaaattcttctcagtcctaacttttcatactaagcccagtccttccaaagcagactgtgaaagagtgatagttccgggagactagcaccggctagccgagcttggaacactttcccttcattaagaaccatccttgctactcagctgcaatcaatccagcccccaggtcttcactgaaccttttcccatctcttccaaaacatctgtttctgagaagtcctgtcctatagaggtctttcttcccaccggatttctcctacaccatttactcccacttgcagaactcccgtgtacaagtgtctttactgcttttatttgctcaacaaaatgcacatctcatataaaaataaatgaggagcatgcacacaccacaaacacaaacaggcatgcagaaatacacatacacacttccctcaatataaaccctttgtggctcatatatttaaaaagatgtaaaaaaaagagctgaagaaaatcatgtgtgatctctcagcagaatagatttattatttgtattgcttgcagaataaagcctatccttgaaagctctgaatcatgggcaagaggctcagtggtatctggaggacagggcactggccactgcagtcaccatcttctgccaggaagcctgcacctcaggggtgaattctttgccaaagtgaatggccagcacggtgaccagcacgttgcccaggagctgtgggaggaagataagagatatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagaaatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtaccctgttacttctccccttcctatgacatgaacttaaccatagaaaagaaggggaaagaaaacatcaagggtcccatagactcaccttgaagttctcaggatccacatgcagcttgtcacagtgcagttcactcagctgggcaaaggtgcccttgagatcatccaggtgctttatggcatctcccaaggaagtcagcaccttcttgccatgtgccttgactttggggttgcccatgatggcagaggcagaggacaggttgccaaagctgtcaaagaacctctgggtccatgggtagacaaccaggagcctgtgagattgacaagaacagtttgacagtcagaaggtgccacaaatcctgagaagcaacctggacttttgccaggcacagggtccttccttccctcccttgtcctggtcaccagagcctaccttcccagggtttctcctccagcatcttccacattcaccttgtcccacaggcttgtgatagtagccttgtcctcctctgtgaaatgacccatggtgtctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttacgacggtatcgatgtatgtgagcatgtgtcctctaacagcacaggccttttgccacctagctgtccaggggtgccttaaaatggcaaacaaggtttgttttcttttcctgttttcatgccttcctcttccatatccttgtttcatattaatacatgtgtatagatcctaaaaatctatacacatgtattaataaagcctgattctgccgcttctaggtatagaggccacctgcaagataaatatttgattcacaataactaatcattctatggcaattgataacaacaaatatatatatatatatatatatacgtatatgtgtatatatatatatatattcaggaaataatatattctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaagggaattccggtgccctgcttaggagcttaatctttaatgaaagctaagctttcattaaaaaaagtctaaccagctgcattcgactttgactgcagcagctggttagaaggttctactggaggagggtcccagcccattgctaaattaacatcaggctctgagactggcagtatatctctaacagtggttgatgctatcttctggaacttgcctgctacattgagaccactgacccatacataggaagcccatagctctgtcctgaactgttaggccactggtccagagagtgtgcatctcctttgatcctcataataaccctatgagatagacacaattattactcttactttatagatgatgatcctgaaaacataggagtcaaggcacttgcccctagctgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcctgctcccaaatttacagtcatagacttcttcatggctgtctcctttatccacagaatgattcctttgcttcattgccccatccatctgatcctcctcatcagtgcagcacagggcccatgagcagtagctgcagagtctcacataggtctggcactgcctctgacatgtccgaccttaggcaaatgcttgactcttctgagctcggatcccttgagctcaggaggtcaaggctgcagtgagacatgatcttgccactgcactccagcctggacagcagagtgaaaccttgcctcacgaaacagaatacaaaaacaaacaaacaaaaaactgctccgcaatgcgcttccttgatgctctaccacataggtctgggtactttgtacacattatctcattgctgttcataattgttagattaattttgtaatattgatattattcctagaaagctgaggcctcaagatgataacttttattttctggacttgtaatagctttctcttgtattcaccatgttgtaactttcttagagtagtaacaatataaagttattgtgagtttttgcaaacacagcaaacacaacgacccatatagacattgatgtgaaattgtctattgtcaatttatgggaaaacaagtatgtactttttctactaagccattgaaacaggaataacagaacaagattgaaagaatacattttccgaaattacttgagtattatacaaagacaagcacgtggacctgggaggagggttattgtccatgactggtgtgtggagacaaatgcaggtttataatagatgggatggcatctagcgcaatgactttgccatcacttttagagagctcttgggggccccagtacacaagaggggacgcagggtatatgtagacatctcattctttttcttagtgtgagaataagaatagccatgacctgagtttatagacaatgagcccttttctctctcccactcagcagctatgagatggcttgccctgcctctctactaggctgactcactccaaggcccagcaatgggcagggctctgtcagggctttgatagcactatctgcagagccagggccgagaaggggtggactccagagactctccctcccattcccgagcagggtttgcttatttatgcatttaaatgatatatttattttaaaagaaataacaggagactgcccagccctggctgtgacatggaaactatgtagaatattttgggttccatttttttttccttctttcagttagaggaaaaggggctcactgcacatacactagacagaaagtcaggagctttgaatccaagcctgatcatttccatgtcatactgagaaagtccccacccttctctgagcctcagtttctctttttataagtaggagtctggagtaaatgatttccaatggctctcatttcaatacaaaatttccgtttattaaatgcatgagcttccgttactccaagactgagaaggaaattgaacctgagactcattgactggcaagatgtccccagaggctctcattcagcaataaaattctcaccttcacccaggcccactagtgtcagatttgcatgcgttcgcgtatcgacgtgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggtacctcaggatatgcccttgactatttgtccgacatagtcaagggcatatccttttttgtgcggccgcatcgatgccgtagtacctttaagaccaatgacttacaaggcagctgtagatcttagccactttttaaaagaaaaggggggactggaagggctaattcactcccaaagaagacaagatccctgcaggcattcaaggccaggctggatgtggctctgggcagcctgggctgctggttgatgaccctgcacatagcagggggttggatctggatgagcactgtgctcctttgcaacccaggccgttctatgattctgtcattctaaatctctctttcagcctaaagctttttccccgtatcccccctggtgtctgctggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctccccagatctgctttttgcctgtactgggtctctctggttagaccagatctgagcctgggagctctctggctaactagggaacccactgcttaagcctcaataaagcttcagctgctcgagctagcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattc

SEQ ID NO: 112 - DNA, 12084 bp, Artificial Sequence
  Synthetic
ggccgcctcggccaaacagcccttgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccagtccctatcagtgatagagaaaagtgaaagtcgagtttaccactccctatcagtgatagagaaaagtgaaagtcgagtttaccactccctatcagtgatagagaaaagtgaaagtcgagctcgccatgggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatactggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcaggatcttcagacctggaaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccataaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggaccgagctcaagcttcgaacgcgtggggatcctctagagtcgagctcgcgaggatcatcaccggtgctagccggagccagaagcaccataagggacatgataagggagccagcagacctctgatctcttcctgaatgctaatcttaaacatcctgaggaagaatgggacttccatttggggtgggcctatgatagggtaataagacagtagtgaatatcaagctacaaaaagccccctttcaaattcttctcagtcctaacttttcatactaagcccagtccttccaaagcagactgtgaaagagtgatagttccgggagactagcaccggctagccgagcttggaacactttcccttcattaagaaccatccttgctactcagctgcaatcaatccagcccccaggtcttcactgaaccttttcccatctcttccaaaacatctgtttctgagaagtcctgtcctatagaggtctttcttcccaccggatttctcctacaccatttactcccacttgcagaactcccgtgtacaagtgtctttactgcttttatttgctcaacaaaatgcacatctcatataaaaataaatgaggagcatgcacacaccacaaacacaaacaggcatgcagaaatacacatacacacttccctcaatataaaccctttgtggctcatatatttaaaaagatgtaaaaaaaagagctgaagaaaatcatgtgtgatctctcagcagaatagatttattatttgtattgcttgcagaataaagcctatccttgaaagctctgaatcatgggcaagaggctcagtggtatctggaggacagggcactggccactgcagtcaccatcttctgccaggaagcctgcacctcaggggtgaattctttgccaaagtgaatggccagcacggtgaccagcacgttgcccaggagctgtgggaggaagataagagatatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagaaatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtaccctgttacttctccccttcctatgacatgaacttaaccatagaaaagaaggggaaagaaaacatcaagggtcccatagactcaccttgaagttctcaggatccacatgcagcttgtcacagtgcagttcactcagctgggcaaaggtgcccttgagatcatccaggtgctttatggcatctcccaaggaagtcagcaccttcttgccatgtgccttgactttggggttgcccatgatggcagaggcagaggacaggttgccaaagctgtcaaagaacctctgggtccatgggtagacaaccaggagcctgtgagattgacaagaacagtttgacagtcagaaggtgccacaaatcctgagaagcaacctggacttttgccaggcacagggtccttccttccctcccttgtcctggtcaccagagcctaccttcccagggtttctcctccagcatcttccacattcaccttgtcccacaggcttgtgatagtagccttgtcctcctctgtgaaatgacccatggtgtctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttacgacggtatcgatgtatgtgagcatgtgtcctctaacagcacaggccttttgccacctagctgtccaggggtgccttaaaatggcaaacaaggtttgttttcttttcctgttttcatgccttcctcttccatatccttgtttcatattaatacatgtgtatagatcctaaaaatctatacacatgtattaataaagcctgattctgccgcttctaggtatagaggccacctgcaagataaatatttgattcacaataactaatcattctatggcaattgataacaacaaatatatatatatatatatatatacgtatatgtgtatatatatatatatattcaggaaataatatattctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaagggaattccggtgccctgcttaggagcttaatctttaatgaaagctaagctttcattaaaaaaagtctaaccagctgcattcgactttgactgcagcagctggttagaaggttctactggaggagggtcccagcccattgctaaattaacatcaggctctgagactggcagtatatctctaacagtggttgatgctatcttctggaacttgcctgctacattgagaccactgacccatacataggaagcccatagctctgtcctgaactgttaggccactggtccagagagtgtgcatctcctttgatcctcataataaccctatgagatagacacaattattactcttactttatagatgatgatcctgaaaacataggagtcaaggcacttgcccctagctgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcctgctcccaaatttacagtcatagacttcttcatggctgtctcctttatccacagaatgattcctttgcttcattgccccatccatctgatcctcctcatcagtgcagcacagggcccatgagcagtagctgcagagtctcacataggtctggcactgcctctgacatgtccgaccttaggcaaatgcttgactcttctgagctcggatcccttgagctcaggaggtcaaggctgcagtgagacatgatcttgccactgcactccagcctggacagcagagtgaaaccttgcctcacgaaacagaatacaaaaacaaacaaacaaaaaactgctccgcaatgcgcttccttgatgctctaccacataggtctgggtactttgtacacattatctcattgctgttcataattgttagattaattttgtaatattgatattattcctagaaagctgaggcctcaagatgataacttttattttctggacttgtaatagctttctcttgtattcaccatgttgtaactttcttagagtagtaacaatataaagttattgtgagtttttgcaaacacagcaaacacaacgacccatatagacattgatgtgaaattgtctattgtcaatttatgggaaaacaagtatgtactttttctactaagccattgaaacaggaataacagaacaagattgaaagaatacattttccgaaattacttgagtattatacaaagacaagcacgtggacctgggaggagggttattgtccatgactggtgtgtggagacaaatgcaggtttataatagatgggatggcatctagcgcaatgactttgccatcacttttagagagctcttgggggccccagtacacaagaggggacgcagggtatatgtagacatctcattctttttcttagtgtgagaataagaatagccatgacctgagtttatagacaatgagcccttttctctctcccactcagcagctatgagatggcttgccctgcctctctactaggctgactcactccaaggcccagcaatgggcagggctctgtcagggctttgatagcactatctgcagagccagggccgagaaggggtggactccagagactctccctcccattcccgagcagggtttgcttatttatgcatttaaatgatatatttattttaaaagaaataacaggagactgcccagccctggctgtgacatggaaactatgtagaatattttgggttccatttttttttccttctttcagttagaggaaaaggggctcactgcacatacactagacagaaagtcaggagctttgaatccaagcctgatcatttccatgtcatactgagaaagtccccacccttctctgagcctcagtttctctttttataagtaggagtctggagtaaatgatttccaatggctctcatttcaatacaaaatttccgtttattaaatgcatgagcttccgttactccaagactgagaaggaaattgaacctgagactcattgactggcaagatgtccccagaggctctcattcagcaataaaattctcaccttcacccaggcccactagtgtcagatttgcatgcgttcgcgtatcgacgtgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggtacctcaggatatgcccttgactatttgtccgacatagtcaagggcatatccttttttgtgcggccgcatcgatgccgtagtacctttaagaccaatgacttacaaggcagctgtagatcttagccactttttaaaagaaaaggggggactggaagggctaattcactcccaaagaagacaagatccctgcaggcattcaaggccaggctggatgtggctctgggcagcctgggctgctggttgatgaccctgcacatagcagggggttggatctggatgagcactgtgctcctttgcaacccaggccgttctatgattctgtcattctaaatctctctttcagcctaaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctccccagatctgctttttgcctgtactgggtctctctggttagaccagatctgagcctgggagctctctggctaactagggaacccactgcttaagcctcaataaagcttcagctgctcgagctagcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattc

SEQ ID NO: 113 - DNA, 12084 bp, Artificial Sequence
  Synthetic
ggccgcctcggccaaacagcccttgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccagtccctatcagtgatagagaaaagtgaaagtcgagtttaccactccctatcagtgatagagaaaagtgaaagtcgagtttaccactccctatcagtgatagagaaaagtgaaagtcgagctcgccatgggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatactggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcaggatcttcagacctggaaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccataaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggaccgagctcaagcttcgaacgcgtggggatcctctagagtcgagctcgcgaggatcatcaccggtgctagccggagccagaagcaccataagggacatgataagggagccagcagacctctgatctcttcctgaatgctaatcttaaacatcctgaggaagaatgggacttccatttggggtgggcctatgatagggtaataagacagtagtgaatatcaagctacaaaaagccccctttcaaattcttctcagtcctaacttttcatactaagcccagtccttccaaagcagactgtgaaagagtgatagttccgggagactagcaccggctagccgagcttggaacactttcccttcattaagaaccatccttgctactcagctgcaatcaatccagcccccaggtcttcactgaaccttttcccatctcttccaaaacatctgtttctgagaagtcctgtcctatagaggtctttcttcccaccggatttctcctacaccatttactcccacttgcagaactcccgtgtacaagtgtctttactgcttttatttgctcaacaaaatgcacatctcatataaaaataaatgaggagcatgcacacaccacaaacacaaacaggcatgcagaaatacacatacacacttccctcaatataaaccctttgtggctcatatatttaaaaagatgtaaaaaaaagagctgaagaaaatcatgtgtgatctctcagcagaatagatttattatttgtattgcttgcagaataaagcctatccttgaaagctctgaatcatgggcaagaggctcagtggtatctggaggacagggcactggccactgcagtcaccatcttctgccaggaagcctgcacctcaggggtgaattctttgccaaagtgaatggccagcacggtgaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagaaatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtaccctgttacttctccccttcctatgacatgaacttaaccatagaaaagaaggggaaagaaaacatcaagggtcccatagactcaccttgaagttctcaggatccacatgcagcttgtcacagtgcagttcactcagctgggcaaaggtgcccttgagatcatccaggtgctttatggcatctcccaaggaagtcagcaccttcttgccatgtgccttgactttggggttgcccatgatggcagaggcagaggacaggttgccaaagctgtcaaagaacctctgggtccatgggtagacaaccaggagcctgtgagattgacaagaacagtttgacagtcagaaggtgccacaaatcctgagaagcaacctggacttttgccaggcacagggtccttccttccctcccttgtcctggtcaccagagcctaccttcccagggtttctcctccagcatcttccacattcaccttgtcccacaggcttgtgatagtagccttgtcctcctctgtgaaatgacccatggtgtctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttacgacggtatcgatgtatgtgagcatgtgtcctctaacagcacaggccttttgccacctagctgtccaggggtgccttaaaatggcaaacaaggtttgttttcttttcctgttttcatgccttcctcttccatatccttgtttcatattaatacatgtgtatagatcctaaaaatctatacacatgtattaataaagcctgattctgccgcttctaggtatagaggccacctgcaagataaatatttgattcacaataactaatcattctatggcaattgataacaacaaatatatatatatatatatatatacgtatatgtgtatatatatatatatattcaggaaataatatattctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaagggaattccggtgccctgcttaggagcttaatctttaatgaaagctaagctttcattaaaaaaagtctaaccagctgcattcgactttgactgcagcagctggttagaaggttctactggaggagggtcccagcccattgctaaattaacatcaggctctgagactggcagtatatctctaacagtggttgatgctatcttctggaacttgcctgctacattgagaccactgacccatacataggaagcccatagctctgtcctgaactgttaggccactggtccagagagtgtgcatctcctttgatcctcataataaccctatgagatagacacaattattactcttactttatagatgatgatcctgaaaacataggagtcaaggcacttgcccctagctgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcctgctcccaaatttacagtcatagacttcttcatggctgtctcctttatccacagaatgattcctttgcttcattgccccatccatctgatcctcctcatcagtgcagcacagggcccatgagcagtagctgcagagtctcacataggtctggcactgcctctgacatgtccgaccttaggcaaatgcttgactcttctgagctcggatcccttgagctcaggaggtcaaggctgcagtgagacatgatcttgccactgcactccagcctggacagcagagtgaaaccttgcctcacgaaacagaatacaaaaacaaacaaacaaaaaactgctccgcaatgcgcttccttgatgctctaccacataggtctgggtactttgtacacattatctcattgctgttcataattgttagattaattttgtaatattgatattattcctagaaagctgaggcctcaagatgataacttttattttctggacttgtaatagctttctcttgtattcaccatgttgtaactttcttagagtagtaacaatataaagttattgtgagtttttgcaaacacagcaaacacaacgacccatatagacattgatgtgaaattgtctattgtcaatttatgggaaaacaagtatgtactttttctactaagccattgaaacaggaataacagaacaagattgaaagaatacattttccgaaattacttgagtattatacaaagacaagcacgtggacctgggaggagggttattgtccatgactggtgtgtggagacaaatgcaggtttataatagatgggatggcatctagcgcaatgactttgccatcacttttagagagctcttgggggccccagtacacaagaggggacgcagggtatatgtagacatctcattctttttcttagtgtgagaataagaatagccatgacctgagtttatagacaatgagcccttttctctctcccactcagcagctatgagatggcttgccctgcctctctactaggctgactcactccaaggcccagcaatgggcagggctctgtcagggctttgatagcactatctgcagagccagggccgagaaggggtggactccagagactctccctcccattcccgagcagggtttgcttatttatgcatttaaatgatatatttattttaaaagaaataacaggagactgcccagccctggctgtgacatggaaactatgtagaatattttgggttccatttttttttccttctttcagttagaggaaaaggggctcactgcacatacactagacagaaagtcaggagctttgaatccaagcctgatcatttccatgtcatactgagaaagtccccacccttctctgagcctcagtttctctttttataagtaggagtctggagtaaatgatttccaatggctctcatttcaatacaaaatttccgtttattaaatgcatgagcttccgttactccaagactgagaaggaaattgaacctgagactcattgactggcaagatgtccccagaggctctcattcagcaataaaattctcaccttcacccaggcccactagtgtcagatttgcatgcgttcgcgtatcgacgtgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggtacctcaggatatgcccttgactatttgtccgacatagtcaagggcatatccttttttgtgcggccgcatcgatgccgtagtacctttaagaccaatgacttacaaggcagctgtagatcttagccactttttaaaagaaaaggggggactggaagggctaattcactcccaaagaagacaagatccctgcaggcattcaaggccaggctggatgtggctctgggcagcctgggctgctggttgatgaccctgcacatagcagggggttggatctggatgagcactgtgctcctttgcaacccaggccgttctatgattctgtcattctaaatctctctttcagcctaaagctttttccccgtatcccccctggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctccccagatctgctttttgcctgtactgggtctctctggttagaccagatctgagcctgggagctctctggctaactagggaacccactgcttaagcctcaataaagcttcagctgctcgagctagcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattc

SEQ ID NO: 114 - DNA, 12084 bp, Artificial Sequence
  Synthetic
ggccgcctcggccaaacagcccttgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccagtccctatcagtgatagagaaaagtgaaagtcgagtttaccactccctatcagtgatagagaaaagtgaaagtcgagtttaccactccctatcagtgatagagaaaagtgaaagtcgagctcgccatgggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatactggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcaggatcttcagacctggaaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccataaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggaccgagctcaagcttcgaacgcgtggggatcctctagagtcgagctcgcgaggatcatcaccggtgctagccggagccagaagcaccataagggacatgataagggagccagcagacctctgatctcttcctgaatgctaatcttaaacatcctgaggaagaatgggacttccatttggggtgggcctatgatagggtaataagacagtagtgaatatcaagctacaaaaagccccctttcaaattcttctcagtcctaacttttcatactaagcccagtccttccaaagcagactgtgaaagagtgatagttccgggagactagcaccggctagccgagcttggaacactttcccttcattaagaaccatccttgctactcagctgcaatcaatccagcccccaggtcttcactgaaccttttcccatctcttccaaaacatctgtttctgagaagtcctgtcctatagaggtctttcttcccaccggatttctcctacaccatttactcccacttgcagaactcccgtgtacaagtgtctttactgcttttatttgctcaacaaaatgcacatctcatataaaaataaatgaggagcatgcacacaccacaaacacaaacaggcatgcagaaatacacatacacacttccctcaatataaaccctttgtggctcatatatttaaaaagatgtaaaaaaaagagctgaagaaaatcatgtgtgatctctcagcagaatagatttattatttgtattgcttgcagaataaagcctatccttgaaagctctgaatcatgggcaagaggctcagtggtatctggaggacagggcactggccactgcagtcaccatcttctgccaggaagcctgcacctcaggggtgaattctttgccaaagtgaatggccagcacggtgaccagcacgttgcccaggagctgtgggaggaagataagagatatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagaaatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtaccctgttacttctccccttcctatgacatgaacttaaccatagaaaagaaggggaaagaaaacatcaagggtcccatagactcaccttgaagttctcaggatccacatgcagcttgtcacagtgcagttcactcagctgggcaaaggtgcccttgagatcatccaggtgctttatggcatctcccaaggaagtcagcaccttcttgccatgtgccttgactttggggttgcccatgatggcagaggcagaggacaggttgccaaagctgtcaaagaacctctgggtccatgggtagacaaccaggagcctgtgagattgacaagaacagtttgacagtcagaaggtgccacaaatcctgagaagcaacctggacttttgccaggcacagggtccttccttccctcccttgtcctggtcaccagagcctaccttcccagggtttctcctccagcatcttccacattcaccttgtcccacaggcttgtgatagtagccttgtcctcctctgtgaaatgacccatggtgtctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttacgacggtatcgatgtatgtgagcatgtgtcctctaacagcacaggccttttgccacctagctgtccaggggtgccttaaaatggcaaacaaggtttgttttcttttcctgttttcatgccttcctcttccatatccttgtttcatattaatacatgtgtatagatcctaaaaatctatacacatgtattaataaagcctgattctgccgcttctaggtatagaggccacctgcaagataaatatttgattcacaataactaatcattctatggcaattgataacaacaaatatatatatatatatatatatacgtatatgtgtatatatatatatatattcaggaaataatatattctagaatatgtcacattctgtctcaggcatccattttctttatgatgccgtttgaggtggagttttagtcaggtggtcagcttctccttttttttgccatctgccctgtaagcatcctgctggggacccagataggagtcatcactctaggctgagaacatctgggcacacaccctaagcctcagcatgactcatcatgactcagcattgctgtgcttgagccagaaggtttgcttagaaggttacacagaaccagaaggcgggggtggggcactgaccccgacaggggcctggccagaactgctcatgcttggactatgggaggtcactaatggagacacacagaaatgtaacaggaactaagggaattccggtgccctgcttaggagcttaatctttaatgaaagctaagctttcattaaaaaaagtctaaccagctgcattcgactttgactgcagcagctggttagaaggttctactggaggagggtcccagcccattgctaaattaacatcaggctctgagactggcagtatatctctaacagtggttgatgctatcttctggaacttgcctgctacattgagaccactgacccatacataggaagcccatagctctgtcctgaactgttaggccactggtccagagagtgtgcatctcctttgatcctcataataaccctatgagatagacacaattattactcttactttatagatgatgatcctgaaaacataggagtcaaggcacttgcccctagctgggggtataggggagcagtcccatgtagtagtagaatgaaaaatgctgctatgctgtgcctcccccacctttcccatgtctgccctctactcatggtctatctctcctggctcctgggagtcatggactccacccagcaccaccaacctgacctaaccacctatctgagcctgccagcctataacccatctgggccctgatagctggtggccagccctgaccccaccccaccctccctggaacctctgatagacacatctggcacaccagctcgcaaagtcaccgtgagggtcttgtgtttgctgagtcaaaattccttgaaatccaagtccttagagactcctgctcccaaatttacagtcatagacttcttcatggctgtctcctttatccacagaatgattcctttgcttcattgccccatccatctgatcctcctcatcagtgcagcacagggcccatgagcagtagctgcagagtctcacataggtctggcactgcctctgacatgtccgaccttaggcaaatgcttgactcttctgagctcggatcccttgagctcaggaggtcaaggctgcagtgagacatgatcttgccactgcactccagcctggacagcagagtgaaaccttgcctcacgaaacagaatacaaaaacaaacaaacaaaaaactgctccgcaatgcgcttccttgatgctctaccacataggtctgggtactttgtacacattatctcattgctgttcataattgttagattaattttgtaatattgatattattcctagaaagctgaggcctcaagatgataacttttattttctggacttgtaatagctttctcttgtattcaccatgttgtaactttcttagagtagtaacaatataaagttattgtgagtttttgcaaacacagcaaacacaacgacccatatagacattgatgtgaaattgtctattgtcaatttatgggaaaacaagtatgtactttttctactaagccattgaaacaggaataacagaacaagattgaaagaatacattttccgaaattacttgagtattatacaaagacaagcacgtggacctgggaggagggttattgtccatgactggtgtgtggagacaaatgcaggtttataatagatgggatggcatctagcgcaatgactttgccatcacttttagagagctcttgggggccccagtacacaagaggggacgcagggtatatgtagacatctcattctttttcttagtgtgagaataagaatagccatgacctgagtttatagacaatgagcccttttctctctcccactcagcagctatgagatggcttgccctgcctctctactaggctgactcactccaaggcccagcaatgggcagggctctgtcagggctttgatagcactatctgcagagccagggccgagaaggggtggactccagagactctccctcccattcccgagcagggtttgcttatttatgcatttaaatgatatatttattttaaaagaaataacaggagactgcccagccctggctgtgacatggaaactatgtagaatattttgggttccatttttttttccttctttcagttagaggaaaaggggctcactgcacatacactagacagaaagtcaggagctttgaatccaagcctgatcatttccatgtcatactgagaaagtccccacccttctctgagcctcagtttctctttttataagtaggagtctggagtaaatgatttccaatggctctcatttcaatacaaaatttccgtttattaaatgcatgagcttccgttactccaagactgagaaggaaattgaacctgagactcattgactggcaagatgtccccagaggctctcattcagcaataaaattctcaccttcacccaggcccactagtgtcagatttgcatgcgttcgcgtatcgacgtgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggtacctcaggatatgcccttgactatttgtccgacatagtcaagggcatatccttttttgtgcggccgcatcgatgccgtagtacctttaagaccaatgacttacaaggcagctgtagatcttagccactttttaaaagaaaaggggggactggaagggctaattcactcccaaagaagacaagatccctgcaggcattcaaggccaggctggatgtggctctgggcagcctgggctgctggttgatgaccctgcacatagcagggggttggatctggatgagcactgtgctcctttgcaacccaggccgttctatgattctgtcattctaaatctctctttcagcctaaagctttttccccgtatcccccctggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctccccagatctgctttttgcctgtactgggtctctctggttagaccagatctgagcctgggagctctctggctaactagggaacccactgcttaagcctcaataaagcttcagctgctcgagctagcagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctgtcgactgcagaggcctgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattc

SEQ ID NO: 115 - DNA, 84 bp, Artificial Sequence
  Synthetic
ggtgtctgtttgaggttgctagtgaacacagttgtgtcagaagcaaatgtaagcaatagatggctctgccctgacttttatgcc

SEQ ID NO: 116 - DNA, 265 bp, Artificial Sequence
  Synthetic
aagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttac

SEQ ID NO: 117 - DNA, 317 bp, Artificial Sequence
  Synthetic
ggtgtctgtttgaggttgctagtgaacacagttgtgtcagaagcaaatgtaagcaatagatggctctgccctgacttttatgcccagccctggctcctgccctccctgctcctgggagtagattggccaaccctagggtgtggctccacagggtgaggtctaagtgatgacagccgtacctgtccttggctcttctggcactggcttaggagttggacttcaaaccctcagccctccctctaagatatatctcttggccccataccatcagtacaaattgctactaaaaacatcctcctttgcaagtgtatttacga

SEQ ID NO: 118 - DNA, 1043 bp, Artificial Sequence
  Synthetic
atgggtcatttcacagaggaggacaaggctactatcacaagcctgtgggacaaggtgaatgtggaagatgctggaggagaaaccctgggaaggtaggctctggtgaccaggacaagggagggaaggaaggaccctgtgcctggcaaaagtccaggttgcttctcaggatttgtggcaccttctgactgtcaaactgttcttgtcaatctcacaggctcctggttgtctacccatggacccagaggttctttgacagctttggcaacctgtcctctgcctctgccatcatgggcaaccccaaagtcaaggcacatggcaagaaggtgctgacttccttgggagatgccataaagcacctggatgatctcaagggcacctttgcccagctgagtgaactgcactgtgacaagctgcatgtggatcctgagaacttcaaggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtcaccgtgctggccattcactttggcaaagaattcacccctgaggtgcaggcttcctggcagaagatggtgactgcagtggccagtgccctgtcctccagataccactgag

SEQ ID NO: 119 - DNA, 1043 bp, Artificial Sequence
  Synthetic
ctcagtggtatctggaggacagggcactggccactgcagtcaccatcttctgccaggaagcctgcacctcaggggtgaattctttgccaaagtgaatggccagcacggtgaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagaaatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtaccctgttacttctccccttcctatgacatgaacttaaccatagaaaagaaggggaaagaaaacatcaagggtcccatagactcaccttgaagttctcaggatccacatgcagcttgtcacagtgcagttcactcagctgggcaaaggtgcccttgagatcatccaggtgctttatggcatctcccaaggaagtcagcaccttcttgccatgtgccttgactttggggttgcccatgatggcagaggcagaggacaggttgccaaagctgtcaaagaacctctgggtccatgggtagacaaccaggagcctgtgagattgacaagaacagtttgacagtcagaaggtgccacaaatcctgagaagcaacctggacttttgccaggcacagggtccttccttccctcccttgtcctggtcaccagagcctaccttcccagggtttctcctccagcatcttccacattcaccttgtcccacaggcttgtgatagtagccttgtcctcctctgtgaaatgacccat

SEQ ID NO: 120 - DNA, 1043 bp, Artificial Sequence
  Synthetic
ctcagtggtatctggaggacagggcactggccactgcagtcaccatcttctgccaggaagcctgcacctcaggggtgaattctttgccaaagtgaatggccagcacggtgaccagcacgttgcccaggagctgtgggaggaagataagagatatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagaaatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtaccctgttacttctccccttcctatgacatgaacttaaccatagaaaagaaggggaaagaaaacatcaagggtcccatagactcaccttgaagttctcaggatccacatgcagcttgtcacagtgcagttcactcagctgggcaaaggtgcccttgagatcatccaggtgctttatggcatctcccaaggaagtcagcaccttcttgccatgtgccttgactttggggttgcccatgatggcagaggcagaggacaggttgccaaagctgtcaaagaacctctgggtccatgggtagacaaccaggagcctgtgagattgacaagaacagtttgacagtcagaaggtgccacaaatcctgagaagcaacctggacttttgccaggcacagggtccttccttccctcccttgtcctggtcaccagagcctaccttcccagggtttctcctccagcatcttccacattcaccttgtcccacaggcttgtgatagtagccttgtcctcctctgtgaaatgacccat

SEQ ID NO: 121 - DNA, 1826 bp, Artificial Sequence
  Synthetic
atgggtcatttcacagaggaggacaaggctactatcacaagcctgtgggacaaggtgaatgtggaagatgctggaggagaaaccctgggaaggtaggctctggtgaccaggacaagggagggaaggaaggaccctgtgcctggcaaaagtccaggttgcttctcaggatttgtggcaccttctgactgtcaaactgttcttgtcaatctcacaggctcctggttgtctacccatggacccagaggttctttgacagctttggcaacctgtcctctgcctctgccatcatgggcaaccccaaagtcaaggcacatggcaagaaggtgctgacttccttgggagatgccataaagcacctggatgatctcaagggcacctttgcccagctgagtgaactgcactgtgacaagctgcatgtggatcctgagaacttcaaggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtcaccgtgctggccattcactttggcaaagaattcacccctgaggtgcaggcttcctggcagaagatggtgactgcagtggccagtgccctgtcctccagataccactgagcctcttgcccatgattcagagctttcaaggataggctttattctgcaagcaatacaaataataaatctattctgctgagagatcacacatgattttcttcagctcttttttttacatctttttaaatatatgagccacaaagggtttatattgagggaagtgtgtatgtgtatttctgcatgcctgtttgtgtttgtggtgtgtgcatgctcctcatttatttttatatgagatgtgcattttgttgagcaaataaaagcagtaaagacacttgtacacgggagttctgcaagtgggagtaaatggtgtaggagaaatccggtgggaagaaagacctctataggacaggacttctcagaaacagatgttttggaagagatgggaaaaggttcagtgaagacctgggggctggattgattgcagctgagtagcaaggatggttcttaatgaagggaaagtgttccaagctcggctagccggtgctagtctcccggaactatcactctttcacagtctgctttggaaggactgggcttagtatgaaaagttaggactgagaagaatttgaaagggggctttttgtagcttgatattcactactgtcttattaccctatcataggcccaccccaaatggaagtcccattcttcctcaggatgtttaagattagcattcaggaagagatcagaggtctgctggctcccttatcatgtcccttatggtgcttctggctccggctagcaccggtgatgatcctcgcgagctcgactctagaggatcccc

SEQ ID NO: 122 - DNA, 1826 bp, Artificial Sequence
  Synthetic
ggggatcctctagagtcgagctcgcgaggatcatcaccggtgctagccggagccagaagcaccataagggacatgataagggagccagcagacctctgatctcttcctgaatgctaatcttaaacatcctgaggaagaatgggacttccatttggggtgggcctatgatagggtaataagacagtagtgaatatcaagctacaaaaagccccctttcaaattcttctcagtcctaacttttcatactaagcccagtccttccaaagcagactgtgaaagagtgatagttccgggagactagcaccggctagccgagcttggaacactttcccttcattaagaaccatccttgctactcagctgcaatcaatccagcccccaggtcttcactgaaccttttcccatctcttccaaaacatctgtttctgagaagtcctgtcctatagaggtctttcttcccaccggatttctcctacaccatttactcccacttgcagaactcccgtgtacaagtgtctttactgcttttatttgctcaacaaaatgcacatctcatataaaaataaatgaggagcatgcacacaccacaaacacaaacaggcatgcagaaatacacatacacacttccctcaatataaaccctttgtggctcatatatttaaaaagatgtaaaaaaaagagctgaagaaaatcatgtgtgatctctcagcagaatagatttattatttgtattgcttgcagaataaagcctatccttgaaagctctgaatcatgggcaagaggctcagtggtatctggaggacagggcactggccactgcagtcaccatcttctgccaggaagcctgcacctcaggggtgaattctttgccaaagtgaatggccagcacggtgaccagcacgttgcccaggagctgtgggaggaagataagaggtatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagaaatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtaccctgttacttctccccttcctatgacatgaacttaaccatagaaaagaaggggaaagaaaacatcaagggtcccatagactcaccttgaagttctcaggatccacatgcagcttgtcacagtgcagttcactcagctgggcaaaggtgcccttgagatcatccaggtgctttatggcatctcccaaggaagtcagcaccttcttgccatgtgccttgactttggggttgcccatgatggcagaggcagaggacaggttgccaaagctgtcaaagaacctctgggtccatgggtagacaaccaggagcctgtgagattgacaagaacagtttgacagtcagaaggtgccacaaatcctgagaagcaacctggacttttgccaggcacagggtccttccttccctcccttgtcctggtcaccagagcctaccttcccagggtttctcctccagcatcttccacattcaccttgtcccacaggcttgtgatagtagccttgtcctcctctgtgaaatgacccat

SEQ ID NO: 123 - DNA, 1826 bp, Artificial Sequence
  Synthetic
ggggatcctctagagtcgagctcgcgaggatcatcaccggtgctagccggagccagaagcaccataagggacatgataagggagccagcagacctctgatctcttcctgaatgctaatcttaaacatcctgaggaagaatgggacttccatttggggtgggcctatgatagggtaataagacagtagtgaatatcaagctacaaaaagccccctttcaaattcttctcagtcctaacttttcatactaagcccagtccttccaaagcagactgtgaaagagtgatagttccgggagactagcaccggctagccgagcttggaacactttcccttcattaagaaccatccttgctactcagctgcaatcaatccagcccccaggtcttcactgaaccttttcccatctcttccaaaacatctgtttctgagaagtcctgtcctatagaggtctttcttcccaccggatttctcctacaccatttactcccacttgcagaactcccgtgtacaagtgtctttactgcttttatttgctcaacaaaatgcacatctcatataaaaataaatgaggagcatgcacacaccacaaacacaaacaggcatgcagaaatacacatacacacttccctcaatataaaccctttgtggctcatatatttaaaaagatgtaaaaaaaagagctgaagaaaatcatgtgtgatctctcagcagaatagatttattatttgtattgcttgcagaataaagcctatccttgaaagctctgaatcatgggcaagaggctcagtggtatctggaggacagggcactggccactgcagtcaccatcttctgccaggaagcctgcacctcaggggtgaattctttgccaaagtgaatggccagcacggtgaccagcacgttgcccaggagctgtgggaggaagataagagatatgaacatgattagcaaaagggcctagcttggactcagaataatccagccttatcccaaccataaaataaaagcagaatggtagctggattgtagctgctattagcaatatgaaacctcttacatcagttacaatttatatgcagaaatatttatatgcagaaatattgctattgccttaacccagaaattatcactgttattctttagaatggtgcaaagaggcatgatacattgtatcattattgccctgaaagaaagagattagggaaagtattagaaataagataaacaaaaaagtatattaaaagaagaaagcattttttaaaattacaaatgcaaaattaccctgatttggtcaatatgtgtaccctgttacttctccccttcctatgacatgaacttaaccatagaaaagaaggggaaagaaaacatcaagggtcccatagactcaccttgaagttctcaggatccacatgcagcttgtcacagtgcagttcactcagctgggcaaaggtgcccttgagatcatccaggtgctttatggcatctcccaaggaagtcagcaccttcttgccatgtgccttgactttggggttgcccatgatggcagaggcagaggacaggttgccaaagctgtcaaagaacctctgggtccatgggtagacaaccaggagcctgtgagattgacaagaacagtttgacagtcagaaggtgccacaaatcctgagaagcaacctggacttttgccaggcacagggtccttccttccctcccttgtcctggtcaccagagcctaccttcccagggtttctcctccagcatcttccacattcaccttgtcccacaggcttgtgatagtagccttgtcctcctctgtgaaatgacccat

(19) United States
(12) Patent Application Publication
(10) Pub. No.: US 2025/0354166 A1
(43) Pub. Date: Nov. 20, 2025

(54) LENTIVIRAL VECTORS USEFUL FOR THE TREATMENT OF DISEASE

(71) Applicants: CSL Behring L.L.C., King of Prussia, PA (US); Seattle Children's Hospital d/b/a Seattle Children's Research Institute, Seattle, WA (US); United States of America, as represented by Secretary, Dept. of HHS of NIH, Bethesda, MD (US)

(72) Inventors: Chao-Guang CHEN, Parkville (AU); Christian MONTELLESE, Zurich (CH); Florian AESCHIMANN, Bern (CH); David J. RAWLINGS, Seattle, WA (US); Iram Fatima KHAN, Issaquah, WA (US); Esther Yu-Tin CHEN, Pasadena, CA (US); Harry MALECH, Bethesda, MD (US); Suk See DERAVIN, Bethesda, MD (US)

(21) Appl. No.: 18/288,358

(22) PCT Filed: Apr. 26, 2022
(86) PCT No.: PCT/US2022/026409

§ 371 Date: Oct. 25, 2023

Related U.S. Application Data
(60) Provisional application No. 63/179,993, filed on Apr. 26, 2021.
(60) Provisional application No. 63/180,001, filed on Apr. 26, 2021.

(51) Int. Cl. C12N 15/86; A61P 7/00; C07K 14/47

(52) U.S. Cl. CPC C12N 15/86; A61P 7/00; C07K 14/47; C12N 2740/15043; C12N 2830/40; C12N 2830/48

(57) ABSTRACT

This disclosure relates generally to lentiviral vectors useful for the treatment of a disease or condition, for example, Wiskott-Aldrich Syndrome (WAS) or Sickle Cell Disease (SCD).

### RELATED APPLICATIONS

This application is a national stage application under 35 U.S.C. § 371 of International Application No. PCT/US2022/026409, filed internationally on Apr. 26, 2022, which claims priority to U.S. Provisional Application No. 63/179,993 entitled “Lentiviral vectors useful for the treatment of Wiskott Aldrich Syndrome” filed Apr. 26, 2021 and U.S. Provisional Application No. 63/180,001 entitled “Lentiviral vectors useful for the treatment of Sickle Cell Disease” filed Apr. 26, 2021, the contents of which are hereby incorporated herein by reference in their entirety.

### INCORPORATION BY REFERENCE OF SEQUENCE LISTING

The present application is being filed along with a Sequence Listing in electronic format. The Sequence Listing is provided as a file entitled 229752008700SUB2SEQLIST.txt, created Jun. 11, 2025, which is 321,495 bytes in size. The information in the electronic format of the Sequence Listing is incorporated by reference in its entirety.

### FIELD OF THE INVENTION

This disclosure relates generally to lentiviral vectors useful for the treatment of a disease or condition, for example, Wiskott-Aldrich Syndrome (WAS) or Sickle Cell Disease (SCD).

### BACKGROUND OF THE INVENTION

Wiskott-Aldrich Syndrome (WAS) is a rare, X-linked primary immunodeficiency (PID) disorder characterized by recurrent infections, small platelets, microthrombocytopenia, eczema, and increased risk of autoimmune manifestations and tumors. Mutations in the Wiskott-Aldrich Syndrome protein (WASP) gene are responsible for Wiskott-Aldrich Syndrome. The gene that encodes the WAS protein is located in the short arm of X chromosome (XP11.22-11.23) and is about 9 kb, including 12 exons, and encoding 502 amino acids. To date, WASP mutations, including missense/nonsense, splicing, small deletions, small insertions, gross deletions, and gross insertions have been identified in patients with Wiskott-Aldrich Syndrome

Wiskott-Aldrich Syndrome protein is a hematopoietic system-specific intracellular signal transduction molecule, which is proline rich, and expressed only in hematopoietic cell lines. Wiskott-Aldrich Syndrome protein is believed to be an important regulator of the actin cytoskeleton found to be expressed in all leukocytes. It is believed to be involved in dynamic cytoskeletal changes, which are essential for multiple cellular functions such as adhesion, migration, phagocytosis, immune synapse formation, and receptor-mediated cellular activation processes (e.g. B and T cell antigen receptors). As a result, both innate and cellular adaptive immunity are believed to be affected in Wiskott-Aldrich Syndrome patients, rendering these patients highly susceptible to infections.

In general, WAS gene mutations that cause absent protein expression result in “classic Wiskott-Aldrich Syndrome.” Reduced Wiskott-Aldrich Syndrome protein expression results in X-linked thrombocytopenia. Wiskott-Aldrich Syndrome protein activating gain-of-function mutations result in X-linked neutropenia. Depending on the mutations within the WAS gene product, there is wide variability of clinical disease. In one study of 154 patients with Wiskott-Aldrich Syndrome, only 30% had the classic presentation with thrombocytopenia, small platelets, eczema, and immunodeficiency; 84% had clinical signs and symptoms of thrombocytopenia, 80% had eczema, 20% had only hematologic abnormalities, and 5% had only infectious manifestations (see Sullivan (1994), J Pediatr., 125(6 Pt 1):876-85). Autoimmune disease is common and occurs in up to 40-70% of patients. There is also believed to be a significantly increased risk of lymphoreticular malignancy (10-20%), such as lymphoma, leukemia, and myelodysplasia. Another review of 55 patients with Wiskott-Aldrich Syndrome from a single hospital in France, over a course of 20 years, found autoimmune or inflammatory conditions in 70% of patients, most commonly autoimmune hemolytic anemia.

Wiskott-Aldrich Syndrome was one of the first conditions ever to be successfully treated by allogeneic hematopoietic stem cell transplantation (HSCT) nearly 40 years ago (Galy, Roncarolo et al. (2008), Expert Opinion on Biological Therapy, Vol. 8(2): pp. 181-190; Candotti (2018), Journal of Clinical Immunology, 33: pp. 13-27). Gene therapy approaches for treatment of WAS continue to be reported, including, for example, Aiuti et al. (2013), Science, 341, p. 1233151; Hacein-Bey Abina, et al. (2015), JAMA, 313, pp. 1550-1563; Koldej et al. (2013), Human Gene Therapy Clinical Development, Vol 24, pp. 77-85; Wielgosz et al. (2015), Molecular Therapy: Methods & Clinical Development Vol 2, pp. 14063 and Singh et al. (2017), Molecular Therapy: Methods & Clinical Development Vol. 4 pp. 1-16.

It is believed that a bone marrow transplant remains the only proven cure for this disease and the outcome is reasonably good for those patients with HLA-matched donors (only available for less than 20% of patients). Hematopoietic stem cell gene therapy (HSC-GT) offers a new, potentially curative, option for patients lacking a matched donor. Gene therapy offers several potential advantages over allogeneic HSCT. It is theoretically available to all patients and is believed to decrease the risks of graft rejection, and possibly avoid the risks associated with Graft versus Host Disease (GvHD).

While clinical trials of HSC-GT using integrating viral vectors, such as lentiviral vectors, for the treatment of WAS have indicated that this approach can be therapeutically effective, patients in a clinical trial using gamma-retroviral vector developed leukemia, resulting from integration events (see e.g. Braun et al. (2014), Sci Transl Med. 6(227):227ra33). This highlights the continued need to develop lentiviral vectors having improved safety profiles.

### SUMMARY OF THE INVENTION

Cryptic splice sites within lentiviral vectors (and indeed other viral vectors), can result in alternative splicing of transgene RNA, leading to the production of potentially non-therapeutic truncated transcripts and proteins, and alternative splicing of the lentiviral genomic RNA, leading to truncated virus RNA and potentially non-viable virus. Moreover, cryptic splice sites within lentiviral vectors can lead to alternative splicing of the transcripts from the gene into which the vector genome has integrated. Alternative splicing of transcripts of genes such as HMGA2, into which lentiviral vectors are known to integrate, can result in cells with clonal growth advantages and thus expansion of those cells expressing the alternatively spliced transcripts. This appears to be due, at least in part, to the absence in these truncated or fused transcripts of one or more of the let-7 binding sites that are present in full HMGA2 transcripts, and which are normally bound by the let-7 family of tumor suppressor microRNAs to negatively regulate expression. While HMGA2 is not considered an oncogene, and clonal expansion resulting from overexpression of truncated or fused transcripts results is generally considered benign, the tolerance for even benign cell growth resulting from administration of a therapeutic lentiviral vector is low, for example, when the patients are pediatric patients, such as in the case of the target population for the treatment of WAS. For other genetic diseases as well, it is desirable to treat patients as early as possible to mitigate the effects of the disease and improve quality of life from an early stage, and therefore there is a need to reduce alternative splicing in all gene therapies.

The present disclosure is predicated, at least in part, on the identification of cryptic splice acceptor sites within a cHS4-derived insulator, including the HS4-650 insulator or the HS4-400 insulator, present in a therapeutic lentiviral vector useful for treating a disease or condition including Wiskott Aldrich Syndrome (WAS) or Sickle Cell Disease (SCD). These cryptic splice acceptor sites are located in the reverse, complement sequence of the HS4-650 insulator and HS4-400 insulator (i.e. on the negative or reverse strand). For the HS4-650 insulator, the first cryptic splice acceptor site is termed splice acceptor site 1 (SA1), and is located at nucleotides 385-386 of SEQ ID NO:2 (i.e. splicing occurs between the nucleotide at position 385 and the nucleotide at position 386), where SEQ ID NO:2 is the reverse, complement sequence of the unmodified HS4-650 insulator set forth in SEQ ID NO:1. The second cryptic splice acceptor site is termed splice acceptor site 2 (SA2), and is located at nucleotides 446-447 of SEQ ID NO:2 (i.e. splicing occurs between the nucleotide at position 446 and the nucleotide at position 447), and the third cryptic splice acceptor site is termed splice acceptor site 3 (SA3), and located at nucleotides 456-457 of SEQ ID NO:2 (i.e. splicing occurs between the nucleotide at position 456 and the nucleotide at position 457). For the HS4-400 insulator, SA2 is located at nucleotides 190-191 of SEQ ID NO:90, where SEQ ID NO:90 is the reverse, complement sequence of the unmodified HS4-400 insulator set forth in SEQ ID NO:89, and SA3 is located at nucleotides 200-201 of SEQ ID NO:90.

A 1.2 kb fragment containing hypersensitive site 4 from the chicken β-globin locus (cHS4) is a well-characterized insulator having barrier and enhancer blocking functions. Accordingly, provided herein are lentiviral vectors that contain a modified cHS4-derived insulator, such as a modified HS4-650 or a modified HS4-400 insulator. In particular, provided herein are lentiviral vectors that contain a modified HS4-650 insulator in which one or more of SA1, SA2 and SA3 has been inactivated and lentiviral vectors that contain a modified HS4-400 insulator in which one or both of SA2 and SA3 have been inactivated. The resulting lentiviral vectors therefore can have associated with them a reduced risk of alternative splicing when introduced into a cell, such as a hematopoietic stem cell. The modified HS4-650 insulators can have a mutation relative to a “wild-type” or unmodified HS4-650 insulator that inactivates SA1, SA2 or SA3. Alternatively, the modified HS4-650 insulator may be oriented within the lentiviral vector, and/or relative to the transgene (e.g. WAS transgene), in such a manner so as to effectively inactivate SA1, SA2 and/or SA3, e.g. SA1, SA2 and SA3 are not on the positive or forward strand of the viral RNA and/or the transcript (e.g. WAS transcript). Similarly, the modified HS4-400 insulators can have a mutation relative to a wild-type or unmodified HS4-400 insulator that inactivates one or both of SA2 and SA3. Alternatively, the modified HS4-400 may be oriented within the lentiviral vector, and/or relative to a transgene (e.g. a globin transgene), in such a manner so as to effectively inactivate SA2 and/or SA3, e.g. SA2 and SA3 are not on the positive or forward strand of the viral RNA and/or the transcript (e.g. a globin transcript).

Thus, in one aspect, provided is a lentiviral vector, comprising:
a first promoter operably linked to a first nucleic acid sequence, wherein the first nucleic acid sequence encodes a Wiskott-Aldrich Syndrome protein; and
a modified HS4-650 insulator, wherein:
when present in the vector, the modified HS4-650 insulator comprises an inactivated splice acceptor site 1 (SA1) relative to an unmodified HS4-650 insulator, and wherein:
   SA1 is present in an unmodified HS4-650 insulator at nucleotide positions 385-386 with numbering relative to SEQ ID NO:2, wherein SEQ ID NO:2 is the reverse, complement sequence of the unmodified HS4-650 insulator set forth in SEQ ID NO:1; and/or
   SA1 comprises the sequence TTGCATCCAG\hat{ }ACACCATCAA (SEQ ID NO:60), where \hat{ } represents the splice position.

In some embodiments, the modified HS4-650 insulator comprises, relative to an unmodified HS4-650 insulator, a mutation that inactivates SA1. In one examples, the mutation is a mutation of the A at position 384 (e.g. an A to T mutation) and/or a mutation of the G at position 385, with numbering relative to SEQ ID NO:2. In particular embodiments, the modified HS4-650 insulator comprises the sequence set forth in any one of SEQ ID NOs:3, 12, 21, 30, 39 and 48.

In some examples, the modified HS4-650 insulator further comprises a mutation that inactivates splice acceptor site 2 (SA2) relative to an unmodified HS4-650 insulator, wherein SA2 is present in an unmodified HS4-650 insulator at nucleotide positions 446-447, with numbering relative to SEQ ID NO:2. For example, the mutation may be a mutation of the A at position 445 (e.g. an A to T mutation) and/or a mutation of the G at position 446, with numbering relative to SEQ ID NO:2. In particular embodiments, the modified HS4-650 insulator comprises the sequence set forth in any one of SEQ ID NOs:4, 13, 22, 31, 40 and 49.

In further examples, the modified HS4-650 insulator also comprises a mutation that inactivates splice acceptor site 3 (SA3) relative to an unmodified HS4-650 insulator, wherein SA3 is present in an unmodified HS4-650 insulator at nucleotide positions 456-457, with numbering relative to SEQ ID NO:2, e.g. a mutation of the A at position 455 (e.g. an A to T mutation) and/or a mutation of the G at position 456 with numbering relative to SEQ ID NO:2. In particular examples, the modified HS4-650 insulator comprises the sequence set forth in SEQ ID NOs:5, 6, 14, 15, 23, 24, 32, 33, 41, 42, 50 and 51.

In some embodiments, the modified HS4-650 insulator is in the opposite orientation to the first nucleic acid sequence. In particular embodiments, the first nucleic acid is in the forward orientation and the modified HS4-650 insulator is in the reverse orientation within the lentiviral vector.

In alternative embodiments, the modified HS4-650 insulator is in the same orientation as the first nucleic acid sequence, thereby inactivating SA1. In particular examples, the first nucleic acid and the modified HS4-650 insulator are in the forward orientation within the lentiviral vector.

In another aspect, provided is a lentiviral vector, comprising:
a first promoter operably linked to a first nucleic acid sequence, the first nucleic acid sequence encoding a Wiskott-Aldrich Syndrome protein; and
a modified HS4-650 insulator, wherein:
when present in the vector, the modified HS4-650 insulator comprises an inactivated splice acceptor site 2 (SA2) relative to an unmodified HS4-650 insulator, and wherein:
   SA2 is present in an unmodified HS4-650 insulator at nucleotide positions 446-447, with numbering relative to SEQ ID NO:2, wherein SEQ ID NO:2 is the reverse, complement sequence of the unmodified HS4-650 insulator set forth in SEQ ID NO:1; and/or
   SA2 comprises the sequence ATCCCCCCAG\hat{ }GTGTCTGCAG (SEQ ID NO:61), where \hat{ } represents the splice position.

In some embodiments, the modified HS4-650 insulator comprises, relative to an unmodified HS4-650 insulator, a mutation that inactivates SA2, e.g. is a mutation of the A at position 445 (e.g. A to T mutation) and/or a mutation of the G at position 446, with numbering relative to SEQ ID NO:2. In some examples, the reverse complement sequence of the modified HS4-650 insulator comprises the sequence set forth in any one of SEQ ID NOs:7, 16, 25, 34, 43 and 52.

The modified HS4-650 insulator may also comprise a mutation that inactivates splice acceptor site 1 (SA1) relative to an unmodified HS4-650 insulator, wherein SA1 is present in an unmodified HS4-650 insulator at nucleotide positions nucleotides 385-386, with numbering relative to SEQ ID NO:2. In some examples, the mutation is a mutation of the A at position 384 (e.g. an A to T mutation) and/or a mutation of the G at position 385, with numbering relative to SEQ ID NO:2. In particular embodiments, the modified HS4-650 insulator comprises the sequence set forth in any one of SEQ ID NOs:4, 13, 22, 31, 40 and 49.

The modified HS4-650 insulator may further comprise a mutation that inactivates splice acceptor site 3 (SA3) relative to an unmodified HS4-650 insulator, wherein SA3 is present in an unmodified HS4-650 insulator at nucleotide positions 456-457 with numbering relative to SEQ ID NO:2, e.g. a mutation of the A at position 455 (e.g. an A to T mutation) and/or a mutation of the G at position 456 with numbering relative to SEQ ID NO:2. In some examples, the reverse complement sequence of the modified HS4-650 insulator comprises the sequence set forth in SEQ ID NOs:5, 6, 14, 15, 23, 24, 32, 33, 41, 42, 50 and 51.

In some embodiments of this aspect, the modified HS4-650 insulator is in the opposite orientation to the first nucleic acid sequence. In one example, the first nucleic acid is in the forward orientation and the modified HS4-650 insulator is in the reverse orientation within the lentiviral vector.

In other embodiments of this aspect, the modified HS4-650 insulator is in the same orientation as the first nucleic acid sequence, thereby inactivating SA2. In one example, the first nucleic acid and the modified HS4-650 insulator are in the forward orientation within the lentiviral vector.

In a further aspect, provided is a lentiviral vector, comprising:
a first promoter operably linked to a first nucleic acid sequence, the first nucleic acid sequence encoding a Wiskott-Aldrich Syndrome protein; and
a modified HS4-650 insulator, wherein:
when present in the vector, the modified HS4-650 insulator comprises an inactivated splice acceptor site 3 (SA3) relative to an unmodified HS4-650 insulator, and wherein:
   SA3 is present in an unmodified HS4-650 insulator at nucleotide positions 456-457, with numbering relative to SEQ ID NO:2, wherein SEQ ID NO:2 is the reverse, complement sequence of the unmodified HS4-650 insulator set forth in SEQ ID NO:1; and/or
   SA3 comprises the sequence GTGTCTGCAG\hat{ }GCTCAAAGAG (SEQ ID NO:62), where \hat{ } represents the splice position.

In one example, the modified HS4-650 insulator comprises, relative to an unmodified HS4-650 insulator, a mutation that inactivates SA3. In some embodiments, the mutation is a mutation of the A at position 455 (e.g. an A to T mutation) and/or a mutation of the G at position 456, with numbering relative to SEQ ID NO:2. In one example, the modified HS4-650 insulator comprises the sequence set forth in any one of SEQ ID NOs:9, 18, 27, 36, 45 and 54.

The modified HS4-650 insulator may also comprise a mutation that inactivates splice acceptor site 1 (SA1) relative to an unmodified HS4-650 insulator, wherein SA1 is present in an unmodified HS4-650 insulator at nucleotide positions 385-386 with numbering relative to SEQ ID NO:2. In one example, the mutation is a mutation of the A at position 384 (e.g. an A to T mutation) and/or a mutation of the G at position 385, with numbering relative to SEQ ID NO:2. In some embodiments, the modified HS4-650 insulator comprises the sequence set forth in any one of SEQ ID NOs:14, 23, 32, 41, and 50.

The modified HS4-650 insulator may further comprise a mutation that inactivates splice acceptor site 2 (SA2) relative to an unmodified HS4-650 insulator, wherein SA2 is present in an unmodified HS4-650 insulator at nucleotide positions nucleotides 446-447 with numbering relative to SEQ ID NO:2, e.g. is a mutation of the A at position 445 (e.g. an A to T mutation) and/or a mutation of the G at position 446 with numbering relative to SEQ ID NO:2. In some examples, the reverse complement sequence of the modified HS4-650 insulator comprises the sequence set forth in SEQ ID NOs:6, 8, 15, 17, 24, 26, 33, 35, 42, 44, 51 and 53.

In particular embodiments of this aspect, the modified HS4-650 insulator is in the opposite orientation to the first nucleic acid sequence. In some examples, the first nucleic acid is in the forward orientation and the modified HS4-650 insulator is in the reverse orientation within the lentiviral vector.

In other embodiments of this aspect, the modified HS4-650 insulator is in the same orientation as the first nucleic acid sequence, thereby inactivating SA3. In some examples, the first nucleic acid and the modified HS4-650 insulator are in the forward orientation within the lentiviral vector.

In some embodiments of the aspects described above, the modified HS4-650 insulator is downstream of the first nucleic acid sequence. In further embodiments, the Wiskott-Aldrich Syndrome protein comprises an amino acid sequence set forth in SEQ ID NO: 76 or a sequence having at least 95% sequence identity thereto. In particular examples, the first nucleic acid sequence comprises a sequence set forth in any one of SEQ ID NOs: 73-75 or a sequence having at least 90% sequence identity thereto.

The lentiviral vectors may further comprise a Woodchuck Hepatitis Virus (WHV) Posttranscriptional Regulatory Element (WPRE) between the first nucleic acid sequence and the modified HS4-650 insulator, e.g. one comprising the nucleic acid sequence set forth in any one of SEQ ID NOs: 77-78 or a sequence having at least 95% sequence identity thereto. In some embodiments, the lentiviral vector comprises a sequence selected from the group consisting of: the sequence set forth as nucleotides 3098-6006 of SEQ ID NO:57 or a sequence having at least 90%, 91%, 92%, 93%, 94%, 95%, 96%, 97%, 98% or 99% sequence identity thereto; the sequence set forth as nucleotides 3098-6009 of SEQ ID NO:58 or a sequence having at least 90%, 91%, 92%, 93%, 94%, 95%, 96%, 97%, 98% or 99% sequence identity thereto; and the sequence set forth as nucleotides 3098-6006 of SEQ ID NO:59 or a sequence having at least 90%, 91%, 92%, 93%, 94%, 95%, 96%, 97%, 98% or 99% sequence identity thereto.

In some examples, the first promoter is an MND promoter, e.g. one comprises the nucleic acid sequence set forth in any one of SEQ ID NOs: 72 or a sequence having at least 90% sequence identity thereto. In particular examples, the vectors comprise a sequence selected from the group consisting of: the sequence set forth as nucleotides 2710-6006 of SEQ ID NO:57 or a sequence having at least 90%, 91%, 92%, 93%, 94%, 95%, 96%, 97%, 98% or 99% sequence identity thereto; the sequence set forth as nucleotides 2710-6009 of SEQ ID NO:58 or a sequence having at least 90%, 91%, 92%, 93%, 94%, 95%, 96%, 97%, 98% or 99% sequence identity thereto; and the sequence set forth as nucleotides 2710-6006 of SEQ ID NO:59 or a sequence having at least 90%, 91%, 92%, 93%, 94%, 95%, 96%, 97%, 98% or 99% sequence identity thereto.

The lentiviral vectors may further comprise a second promoter operably linked to a second nucleic acid sequence, wherein the second nucleic acid sequence encodes a nucleic acid that inhibits HPRT expression. In some examples, the nucleic acid that inhibits HPRT expression is a shRNA, e.g. one comprising a hairpin loop sequence set forth in of SEQ ID NO: 66 and/or comprising a nucleic acid sequence set forth in any one of SEQ ID NOs: 67-68 or a sequence comprising at least 95% sequence identity thereto. In some examples, the second promoter comprises a Pol III promoter or a Pol II promoter, e.g. one that comprises 7sk (e.g. one comprising a nucleic acid sequence set forth in any one of SEQ ID NOs:69-71 or a sequence having at least 95% sequence identity thereto). In some examples, the second promoter and the operably linked second nucleic acid sequence are in the reverse orientation and upstream of the first promoter and the operably linked first nucleic acid. In some embodiments, the vectors comprise a sequence selected from the group consisting of: the sequence set forth as nucleotides 2402-6006 of SEQ ID NO:57 or a sequence having at least 90%, 91%, 92%, 93%, 94%, 95%, 96%, 97%, 98% or 99% sequence identity thereto; the sequence set forth as nucleotides 2402-6009 of SEQ ID NO:58 or a sequence having at least 90%, 91%, 92%, 93%, 94%, 95%, 96%, 97%, 98% or 99% sequence identity thereto; and the sequence set forth as nucleotides 2402-6006 of SEQ ID NO:59 or a sequence having at least 90%, 91%, 92%, 93%, 94%, 95%, 96%, 97%, 98% or 99% sequence identity thereto.

The lentiviral vectors may further comprise a polyadenylation signal downstream of the first nucleic acid and the modified HS4-650 insulator.

In some examples, the vector is a plasmid. In other examples, the vector is a viral particle.

Also provided are host cells comprising the lentiviral vector of the present disclosure or transduced with a lentiviral vector of the present disclosure. In some examples, the host cell is a hematopoietic stem cell (HSC), e.g. an allogeneic or autologous HSC.

Also provided are methods for treating a subject with Wiskott-Aldrich Syndrome, comprising administering to the subject the host cell of described above and herein. In particular embodiments, the methods comprise administering to the subject the host cell and then administering a purine analog (e.g. 6-thioguanine (“6TG”), 6-mercaptopurine (“6MP”) or azathiopurine (“AZA”)) to the subject to increase engraftment of the host cell. In further embodiments, the methods comprise pre-conditioning the subject with a purine analog prior to administering the host cell. Also provided are uses of the host cells of the present disclosure for the preparation of a medicament for the treatment of Wiskott-Aldrich Syndrome.

### BRIEF DESCRIPTION OF THE DRAWINGS

Embodiments of the disclosure are described herein, by way of non-limiting example only, with reference to the following drawings.

FIG. 1 is an alignment of the reverse complement sequences of HS4-650 insulators.

FIG. 2 is a schematic of pBRNGTR47.

FIG. 3 is a schematic of pBRNGTR47 showing cryptic splice acceptor sites SA1, SA2 and SA3 in the HS4-650 insulator (650 bp Ins).

FIG. 4 is a schematic of pBRNGTR84.

FIG. 5 is a schematic of pBRNGTR88.

FIG. 6 is a schematic of pBRNGTR92.

FIG. 7 is a schematic of pBRNGTR120.

FIG. 8 shows the ratio of transcripts of HMGA2 exons 2-3/exons 4-5 by ddPCR assessed at day 7 (solid bar, left) and day 14 (right). “650” refers to construct comprising unmodified HS4-650 insulator with unmodified splice sites; “3×SA” refers to construct comprising a HS4-650 insulator with three corrected cryptic splice acceptor sites; “fwd” refers to a construct comprising a HS4-650 insulator in a forward orientation relative to transgene (or a reverse orientation relative to control construct comprising original unmodified insulator); and “mock” refers to control.

FIG. 9 shows edited cells frequency in culture from day 7 (solid bar) to day 26 (hashed bar) showing reduction or elimination of selective cell growth advantage in culture over time for constructs comprising a modified insulator in KG1 cells. “650” refers to construct comprising unmodified HS4-650 insulator with unmodified splice sites; “2×SA” refers to a construct comprising a HS4-650 insulator with two corrected cryptic splice acceptor sites; “3×SA” refers to construct comprising a HS4-650 insulator with three corrected cryptic splice acceptor sites; “fwd” refers to a construct comprising a HS4-650 insulator in a forward orientation relative to transgene (or a reverse orientation relative to control construct comprising original unmodified insulator); and “mock” or “MND-GFP” refer to controls.

FIG. 10 shows edited cells frequency in culture from day 5 (solid bar, left) to day 26 (hashed bar, right) showing reduction or elimination of selective cell growth advantage in culture over time for constructs comprising a modified insulator in CD34+ cells. “650” refers to construct comprising unmodified HS4-650 insulator with unmodified splice sites; “3×SA” refers to construct comprising a HS4-650 insulator with three corrected cryptic splice acceptor sites; “fwd” refers to a construct comprising a HS4-650 insulator in a forward orientation relative to transgene (or a reverse orientation relative to control construct comprising original unmodified insulator); and “mock” or “GFP” refer to controls.

FIG. 11 is a schematic of the mapping of custom baits for enrichment to HMGA2 exons 1, 2 and 3.

FIG. 12 shows the expression level of HMGA2 transcripts and AAV fusion transcripts in KG1 cells. (A) The measure of total HMGA2-expressing transcripts compared to untreated cells. (B) The measure of level of fusion transcripts expressed in cells normalized to the 3×SA. “650” refers to construct comprising unmodified HS4-650 insulator with unmodified splice sites; “2×SA” refers to construct comprising a HS4-650 insulator with two corrected cryptic splice acceptor sites; “3×SA” refers to construct comprising a 650 bp cHS4 insulator with three corrected cryptic splice acceptor sites; “fwd” refers to a construct comprising a HS4-650 insulator in a forward orientation relative to transgene (or a reverse orientation relative to control construct comprising original unmodified insulator); and “mock” or “fwd_LTRrev” refer to controls.

FIG. 13 shows the expression level of HMGA2 transcripts and AAV fusion transcripts in CD34+ cells. (A) The measure of total HMGA2-expressing transcripts compared to untreated cells. (B) The measure of level of fusion transcripts expressed in cells normalized to the 3×SA. “650” refers to construct comprising unmodified HS4-650 insulator with unmodified splice sites; “3×SA” refers to construct comprising a HS4-650 insulator with three corrected cryptic splice acceptor sites; “fwd” refers to a construct comprising a HS4-650 insulator in a forward orientation relative to transgene (or a reverse orientation relative to control construct comprising original unmodified insulator); and “mock” refers to control.

FIG. 14 shows the percentage of exon3-LVV splice junctions mapped from HMGA2 transcript assays in CD34+ cells. “650” refers to construct comprising unmodified HS4-650 insulator with unmodified splice sites; “3×SA” refers to construct comprising a HS4-650 insulator with three corrected cryptic splice acceptor sites; “fwd” refers to a construct comprising a HS4-650 insulator in a forward orientation relative to transgene (or a reverse orientation relative to control construct comprising original unmodified insulator); and “mock”, “AAV_only” or “MND_GFP” refer to controls.

FIG. 15 shows the percentage of HMGA2 exon3-exon4 splice junctions mapped from HMGA2 transcript assays in CD34+ cells. “650” refers to construct comprising unmodified HS4-650 insulator with unmodified splice sites; “3×SA” refers to construct comprising a HS4-650 insulator with three corrected cryptic splice acceptor sites; “fwd” refers to a construct comprising a HS4-650 insulator in a forward orientation relative to transgene (or a reverse orientation relative to control construct comprising original unmodified insulator); and “mock”, “AAV_only” or “MND_GFP” refer to controls.

FIG. 16 shows the ratio of LVV fusion transcripts to HMGA2 isoform 1 mapped from HMGA2 transcript assays in CD34+ cells. “650” refers to construct comprising unmodified HS4-650 insulator with unmodified splice sites; “3×SA” refers to construct comprising a HS4-650 insulator with three corrected cryptic splice acceptor sites; “fwd” refers to a construct comprising a HS4-650 insulator in a forward orientation relative to transgene (or a reverse orientation relative to control construct comprising original unmodified insulator); and “mock”, “AAV_only” or “MND_GFP” refer to controls.

FIG. 17 shows the percentage of exon3-LVV splice junctions mapped from HMGA2 transcript assays in KG1 cells. “650” refers to construct comprising unmodified HS4-650 insulator with unmodified splice sites; “3×SA” refers to construct comprising a HS4-650 insulator with three corrected cryptic splice acceptor sites; “fwd” refers to a construct comprising a HS4-650 insulator in a forward orientation relative to transgene (or a reverse orientation relative to control construct comprising original unmodified insulator); and “mock”, “AAV_only” or “MND_GFP” refer to controls.

FIG. 18 shows the percentage of HMGA2 exon3-exon4 splice junctions mapped from HMGA2 transcript assays in KG1 cells. “650” refers to construct comprising unmodified HS4-650 insulator with unmodified splice sites; “3×SA” refers to construct comprising a HS4-650 insulator with three corrected cryptic splice acceptor sites; “fwd” refers to a construct comprising a HS4-650 insulator in a forward orientation relative to transgene (or a reverse orientation relative to control construct comprising original unmodified insulator); and “mock”, “AAV_only” or “MND_GFP” refer to controls.

FIG. 19 shows the ratio of LVV fusion transcripts to HMGA2 isoform 1 mapped from HMGA2 transcript assays in KG1 cells. “650” refers to construct comprising unmodified HS4-650 insulator with unmodified splice sites; “3×SA” refers to construct comprising a HS4-650 insulator with three corrected cryptic splice acceptor sites; “fwd” refers to a construct comprising a HS4-650 insulator in a forward orientation relative to transgene (or a reverse orientation relative to control construct comprising original unmodified insulator); and “mock”, “AAV_only” or “MND_GFP” refer to controls.

FIG. 20 shows the results of a LIM domain only 2 (LMO2) activation assay in single cell assays. LMO2 mRNA levels (%; y-axis) in mScarlet+ cells normalized to PPIA relative to control construct comprising no insulator. “650” refers to construct comprising unmodified HS4-650 insulator with unmodified splice sites; “3×SA” refers to construct comprising a HS4-650 insulator with three corrected cryptic splice acceptor sites; “fwd” refers to a construct comprising a HS4-650 insulator in a forward orientation relative to transgene (or a reverse orientation relative to control construct comprising original unmodified insulator). “Promoter-free” refers to a control construct lacking a promoter; “Insulator-free” refers to a control construct lacking an insulator.

FIG. 21 shows the results of a LMO2 activation assay in bulk cell assays. (A) LMO2 mRNA levels (%; y-axis) in mScarlet+ cells normalized to PPIA relative to control construct comprising no insulator in bulk cell assays. (B) Expanded plot extracted from (A), LMO2 mRNA levels (%; y-axis) in mScarlet+ cells normalized to PPIA relative to control construct comprising no insulator. “650” refers to construct comprising unmodified HS4-650 insulator with unmodified splice sites; “3×SA” refers to construct comprising a HS4-650 insulator with three corrected cryptic splice acceptor sites; “fwd” refers to a construct comprising a HS4-650 insulator in a forward orientation relative to transgene (or a reverse orientation relative to control construct comprising original unmodified insulator). “No-Ins” refers to a control construct lacking an insulator. Note: represents mean data from three independent replicates.

FIG. 22 shows the ratio of AAV/HMGA2 in exemplary constructs including modified or unmodified insulators in (A) KG1 and (B) CD34+ cells, calculated as ratio between AAV reads and HMGA2 downstream exon reads. “650” refers to construct comprising unmodified HS4-650 insulator with unmodified splice sites; “2×SA” refers to construct comprising a HS4-650 insulator with two corrected cryptic splice acceptor sites; “3×SA” refers to construct comprising a HS4-650 insulator with three corrected cryptic splice acceptor sites; “fwd” refers to a construct comprising a HS4-650 insulator in a forward orientation relative to transgene (or a reverse orientation relative to control construct comprising original unmodified insulator); “mock” refers to control; and “(r1)” . . . “(r2)” . . . refers to sample replicate number.

FIG. 23 shows the expression of WAS in Murine linage negative (Lin^{neg}) WAS KO cells transduced with selected WAS LVVs. Transgene expression shown as MFI (y-axis) in cells transduced at a multiplicity of infection (MOI) of 1 and 10 (as indicated). “650” refers to construct comprising unmodified HS4-650 insulator with unmodified splice sites; “3SA” refers to construct comprising a HS4-650 insulator with three corrected cryptic splice acceptor sites; “fwd” refers to a construct comprising a HS4-650 insulator in a forward orientation relative to transgene (or a reverse orientation relative to control construct comprising original unmodified insulator); “KO” refers to untransduced WAS KO cells and “WT” refers to wild-type cells (Lin^{neg} cells) as negative and positive controls.

FIG. 24 shows the expression of WAS in human U937 WAS KO cells transduced with selected WAS LVVs. Transgene expression shown as MFI (y-axis) in cells transduced at a multiplicity of MOI of 1 and 10 (as indicated). “650” refers to construct comprising unmodified HS4-650 insulator with unmodified splice sites; “3SA” refers to construct comprising a HS4-650 insulator with three corrected cryptic splice acceptor sites; “fwd” refers to a construct comprising a HS4-650 insulator in a forward orientation relative to transgene (or a reverse orientation relative to control construct comprising original unmodified insulator); “KO” refers to untransduced WAS KO cells and “WT” refers to wild-type cells (U937 cells) as negative and positive controls.

FIG. 25 shows the dose dependent increase in vector copy integrations (VCN) in Murine Lin^{neg} WAS KO cells transduced with selected WAS LVVs at MOI of 1, 2, 10 and 20 (as indicated). “650” refers to construct comprising unmodified HS4-650 insulator with unmodified splice sites; “3SA” refers to construct comprising a HS4-650 insulator with three corrected cryptic splice acceptor sites; “fwd” refers to a construct comprising a HS4-650 insulator in a forward orientation relative to transgene (or a reverse orientation relative to control construct comprising original unmodified insulator); “KO” refers to untransduced WAS KO cells and “WT” refers to wild-type cells (Lin^{neg} cells) as negative and positive controls.

FIG. 26 shows the dose dependent increase in VCN in human U937 WAS KO cells transduced with selected WAS LVVs at MOI of 1, 2, 10 and 20 (as indicated). “650” refers to construct comprising unmodified HS4-650 insulator with unmodified splice sites; “3SA” refers to construct comprising a HS4-650 insulator with three corrected cryptic splice acceptor sites; “fwd” refers to a construct comprising a HS4-650 insulator in a forward orientation relative to transgene (or a reverse orientation relative to control construct comprising original unmodified insulator). “KO” refers to untransduced WAS KO cells and “WT” refers to wild-type cells (U937 cells) as negative and positive controls.

FIG. 27 shows the arrangement of the genes and elements in pCalH10. (A) High-level overview schematic of pCalH10. (B) Detailed schematic of pCalH10.

FIG. 28 shows the results of a Southern blot analysis of HeLa cells transduced with virion produced from pCalH10. (A) Southern Blot showing size of fragments observed in cells. (B) Quantification of contribution of each fragment to the population.

FIG. 29 is a schematic of pCalH10 showing the location of splice donor site 1 (SD1) and splice acceptor site 1 (SA2) and the fusion produced after alternative splicing at these sites.

FIG. 30 is a schematic of pCalH10 showing the location of splice donor site 1 (SD1), splice acceptor site 2 (SA2) and splice acceptor site 3 (SA3).

### DETAILED DESCRIPTION OF THE INVENTION

### 1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e., to at least one) of the grammatical object of the article. By way of example, “an element” means one element or more than one element.

As used herein, “and/or” refers to and encompasses any and all possible combinations of one or more of the associated listed items, as well as the lack of combinations when interpreted in the alternative (or).

The terms “active agent” and “therapeutic agent” are used interchangeably herein and refer to agents that prevent, reduce or ameliorate at least one symptom of a disease or disorder.

The terms “administration concurrently” or “administering concurrently” or “co-administering” and the like refer to the administration of a single composition containing two or more agents, or the administration of each agent as separate compositions and/or delivered by separate routes either contemporaneously or simultaneously or sequentially within a short enough period of time that the effective result is equivalent to that obtained when all such agents are administered as a single composition. By “simultaneously” is meant that the agents are administered at substantially the same time, and desirably together in the same formulation. By “contemporaneously” it is meant that the agents are administered closely in time, e.g., one agent is administered within from about one minute to within about one day before or after another. Any contemporaneous time is useful. However, it will often be the case that when not administered simultaneously, the agents will be administered within about one minute to within about eight hours and suitably within less than about one to about four hours. When administered contemporaneously, the agents are suitably administered at the same site on the subject. The term “same site” includes the exact location, but can be within about 0.5 to about 15 centimeters, preferably from within about 0.5 to about 5 centimeters. The term “separately” as used herein means that the agents are administered at an interval, for example at an interval of about a day to several weeks or months. The agents may be administered in either order. The term “sequentially” as used herein means that the agents are administered in sequence, for example at an interval or intervals of minutes, hours, days or weeks. If appropriate the agents may be administered in a regular repeating cycle.

Throughout this specification, unless the context requires otherwise, the words “comprise”, “comprises” and “comprising” will be understood to imply the inclusion of a stated step or element or group of steps or elements but not the exclusion of any other step or element or group of steps or elements. Thus, use of the term “comprising” and the like indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

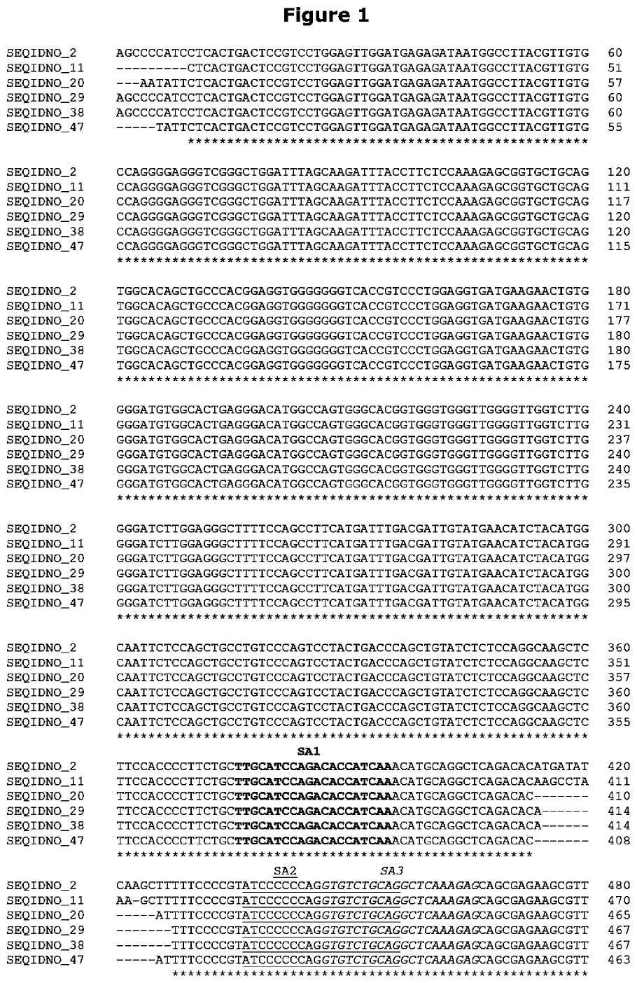

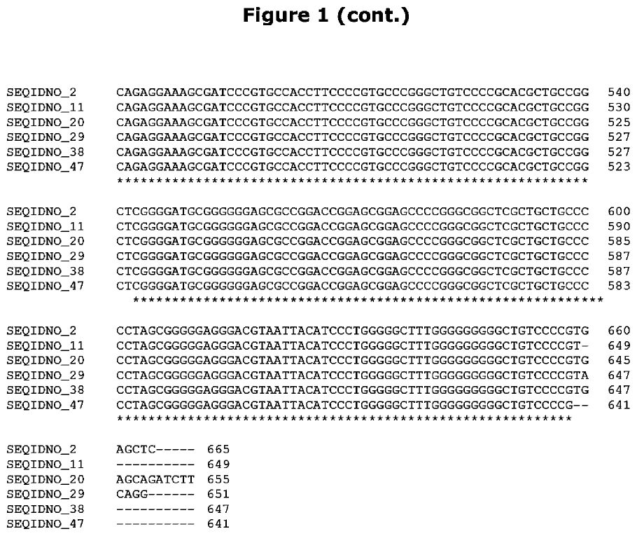

As used herein, “corresponding nucleotides”, “corresponding amino acid residues” or “corresponding positions” refer to nucleotides, amino acids or positions that occur at aligned loci. The sequences of related or variant polynucleotides or polypeptides are aligned by any method known to those of skill in the art. Such methods typically maximize matches (e.g. identical nucleotides or amino acids at positions), and include methods such as using manual alignments and by using the numerous alignment programs available (for example, BLASTN, BLASTP, ClustlW, ClustlW2, EMBOSS, LALIGN, Kalign, etc) and others known to those of skill in the art. By aligning the sequences of polynucleotides, one skilled in the art can identify corresponding nucleotides. For example, by aligning the HS4-650 insulator set forth in SEQ ID NO:2 with other HS4-650 insulators (e.g. as shown in FIG. 1), one of skill in the art can identify regions or nucleotides within the other insulator that correspond to various regions or nucleotides in the insulator set forth in SEQ ID NO:2. For example, the A at position 384 of SEQ ID NO:2 is the corresponding nucleotide of, or corresponds to, the A at position 375 of SEQ ID NO:11. In another example, the SA1 site at nucleotides 385-386 of SEQ ID NO:2 corresponds to the SA1 site at nucleotides 375-376 of SEQ ID NO:20. Thus, when nucleotides or positions are referred to herein with respect to a particular sequence (e.g. an HS4 650 insulator sequence) it is understood that, where appropriate, the reference is also to the corresponding nucleotide or position in another sequence (e.g. another HS4 650 insulator sequence). For example, reference to SA1 in a HS4-650 insulator “at nucleotide positions 385-386, with numbering relative to SEQ ID NO:2” refers to the SA1 at position 385-386 of the HS4-650 insulator set forth in SEQ ID NO:2 and SA1 in other HS4-650 insulators, where the SA1 is at positions corresponding to 385-386 of the HS4-650 insulator set forth in SEQ ID NO:2. In another example, reference to a HS4-650 insulator comprising a mutation of the A at position 384, with numbering relative to SEQ ID NO:2 encompasses not only the HS4-650 insulator set forth in SEQ ID NO:2 having a mutation of the A at position 384, but also other HS4-650 insulators having a mutation of the A at the position that corresponds to position 384 of SEQ ID NO:2.

By “effective amount”, in the context of treating a disease or condition is meant the administration of an amount of an agent or composition to an individual in need of such treatment or prophylaxis, either in a single dose or as part of a series, that is effective for the prevention of incurring a symptom, holding in check such symptoms, and/or treating existing symptoms, of that condition. The effective amount will vary depending upon the age, health and physical condition of the individual to be treated and whether symptoms of disease are apparent, the taxonomic group of individual to be treated, the formulation of the composition, the assessment of the medical situation, and other relevant factors. Optimal dosing schedules can be calculated from measurements of drug accumulation in the body of the subject. Optimum dosages may vary depending on the relative potency in an individual subject, and can generally be estimated based on EC50 values found to be effective in in vitro and in vivo animal models. Persons of ordinary skill can easily determine optimum dosages, dosing methodologies and repetition rates. It is expected that the amount will fall in a relatively broad range that can be determined through routine trials.

The terms “subject”, “patient” and “individual” used interchangeably herein, refer to any subject, particularly a vertebrate subject, and even more particularly a mammalian subject, (e.g. human). In some embodiments, the term “subject” refers to a mammalian subject, (e.g. human) with WAS. In other embodiments, the term “subject” refers to a mammalian subject, (e.g. human) with SCD.

As used herein, the term “expression cassette” refers to one or more genetic sequences within a vector which can express a RNA, and, in some embodiments, subsequently a protein. The expression cassette comprises at least one promoter and at least one gene of interest. In some embodiments, the expression cassette includes at least one promoter, at least one gene of interest, and at least one additional nucleic acid sequence encoding a molecule for expression (e.g. a transgene or RNAi). In some embodiments, the expression cassette is positionally and sequentially oriented within the vector such that the nucleic acid in the cassette can be transcribed into RNA, and when necessary, translated into a protein or a polypeptide, undergo appropriate post-translational modifications required for activity in the transformed cell (e.g. transduced stem cell), and be translocated to the appropriate compartment for biological activity by targeting to appropriate intracellular compartments or secretion into extracellular compartments. In some embodiments, the cassette has its 3′ and 5′ ends adapted for ready insertion into a vector, e.g., it has restriction endonuclease sites at each end.

As used herein, the term “host cell” refers to cells that is to be modified using the methods of the present disclosure. In some embodiments, the host cells are mammalian cells in which the lentiviral vector can be introduced. Suitable mammalian host cells include, but are not limited to, human cells, murine cells, non-human primate cells (e.g. rhesus monkey cells), human progenitor cells or stem cells, 293 cells, HeLa cells, D17 cells, MDCK cells, BHK cells, and Cf2Th cells. In certain embodiments, the host cell comprising an expression vector of the disclosure is a hematopoietic cell, such as hematopoietic progenitor/stem cell (e.g. CD34-positive hematopoietic progenitor/stem cell), a monocyte, a macrophage, a peripheral blood mononuclear cell, a CD4+T lymphocyte, a CD8+T lymphocyte, or a dendritic cell. The hematopoietic cells (e.g. CD4+T lymphocytes, CD8+T lymphocytes, and/or monocyte/macrophages) to be transduced with an expression vector of the disclosure can be allogeneic, autologous, or from a matched sibling. The hematopoietic cells are, in some embodiments, CD34-positive and can be isolated from the patient's bone marrow or peripheral blood. The isolated CD34-positive hematopoietic cells (and/or other hematopoietic cell described herein) is, in some embodiments, transduced with an expression vector as described herein.

As used herein, the term “hematopoietic stem cells” or “HSCs” refer to multipotent cells capable of differentiating into all the cell types of the hematopoietic system, including, but not limited to, granulocytes, monocytes, erythrocytes, megakaryocytes, lymphocytes, dendritic cells; and self-renewal activity, i.e. the ability to divide and generate at least one daughter cell with the identical (e.g., self-renewing) characteristics of the parent cell.

As used herein, “HPRT” is an enzyme involved in purine metabolism encoded by the HPRT1 gene. HPRT1 is located on the X chromosome, and thus is present in single copy in males. HPRT1 encodes the transferase that catalyzes the conversion of hypoxanthine to inosine monophosphate and guanine to guanosine monophosphate by transferring the 5-phosphorobosyl group from 5-phosphoribosyl 1-pyrophosphate to the purine. The enzyme functions primarily to salvage purines from degraded DNA for use in renewed purine synthesis.

As used herein, the term “lentivirus” refers to a genus of retroviruses that are capable of infecting dividing and non-dividing cells. Several examples of lentiviruses include HIV (human immunodeficiency virus: including HIV type 1, and HIV type 2), the etiologic agent of the human acquired immunodeficiency syndrome (AIDS); visna-maedi, which causes encephalitis (visna) or pneumonia (maedi) in sheep, the caprine arthritis-encephalitis virus, which causes immune deficiency, arthritis, and encephalopathy in goats; equine infectious anemia virus, which causes autoimmune hemolytic anemia, and encephalopathy in horses; feline immunodeficiency virus (FIV), which causes immune deficiency in cats; bovine immune deficiency virus (BIV), which causes lymphadenopathy, lymphocytosis, and possibly central nervous system infection in cattle; and simian immunodeficiency virus (SIV), which causes immune deficiency and encephalopathy in sub-human primates.

As used herein, the term “lentiviral vector” is used to denote any form of a nucleic acid derived from a lentivirus and used to transfer genetic material into a cell via transduction. The term encompasses lentiviral vector nucleic acids, such as DNA and RNA, encapsulated forms of these nucleic acids, and viral particles in which the viral vector nucleic acids have been packaged.

As used herein, the term “mutated” refers to a change in a sequence, such as a nucleotide or amino acid sequence, from a native, standard, or reference version of the respective sequence, i.e. the non-mutated sequence.

As used herein, the term “operably linked” refers to functional linkage between a nucleic acid expression control sequence (such as a promoter, signal sequence, enhancer or array of transcription factor binding sites) and a second nucleic acid sequence, wherein the expression control sequence affects transcription and/or translation of the nucleic acid corresponding to the second sequence when the appropriate molecules (e.g., transcriptional activator proteins) are bound to the expression control sequence.

As used herein, the term “promoter” refers to a recognition site of a polynucleotide (DNA or RNA) to which an RNA polymerase binds. An RNA polymerase initiates and transcribes polynucleotides operably linked to the promoter. In some embodiments, promoters operative in mammalian cells comprise an AT-rich region located approximately 25 to 30 bases upstream from the site where transcription is initiated and/or another sequence found about 70 to about 80 bases upstream from the start of transcription, e.g. a CNCAAT region where N may be any nucleotide.

As used herein, the terms “small hairpin RNA” or “shRNA” refer to RNA molecules comprising an antisense region, a loop portion and a sense region, wherein the sense region has complementary nucleotides that base pair with the antisense region to form a duplex stem. Following post-transcriptional processing, the small hairpin RNA is converted into a small interfering RNA by a cleavage event mediated by the enzyme DICER, which is a member of the RNase III family. As used herein, the phrase “post-transcriptional processing” refers to mRNA processing that occurs after transcription and is mediated, for example, by the enzymes DICER and/or Drosha.

As used herein, the terms “transduce” or “transduction” refer to the delivery of a gene(s) using a viral or retroviral vector by means of infection rather than by transfection. For example, an anti-HPRT gene carried by a retroviral vector (a modified retrovirus used as a vector for introduction of nucleic acid into cells) can be transduced into a cell through infection and provirus integration. Thus, a “transduced gene” is a gene that has been introduced into the cell via lentiviral or vector infection and provirus integration. Viral vectors (e.g., “transducing vectors”) transduce genes into “target cells” or host cells.

As used herein, the terms “treatment”, “treating”, and the like, refer to obtaining a desired pharmacologic and/or physiologic effect in a subject in need of treatment, that is, a subject who has a disease or disorder. By “treatment” is meant ameliorating or preventing one or more symptoms or effects (e.g. consequences) of a disease or disorder. Reference to “treatment”, “treat” or “treating” does not necessarily mean to reverse or prevent any or all symptoms or effects of a disease or disorder. For example, the subject may ultimately suffer one or more symptoms or effects, but the number and/or severity of the symptoms or effects is reduced and/or the quality of life is improved compared to prior to treatment.

Each embodiment described herein is to be applied mutatis mutandis to each and every embodiment unless specifically stated otherwise.

TABLE 1
Brief Description of the Sequences

| SEQ | |
|---|---|
| ID NO. | Description |
| 1 | Unmodified HS4-650 insulator |
| 2 | Unmodified HS4-650 insulator − reverse complement (r-c) of SEQ ID NO: 1 |
| 3 | Modified HS4-650 insulator (r-c) − A to T mutation at SA1 |
| 4 | Modified HS4-650 insulator (r-c) − A to T mutations at SA1 and SA2 |
| 5 | Modified HS4-650 insulator (r-c) − A to T mutations at SA1 and SA3 |
| 6 | Modified HS4-650 insulator (r-c) − A to T mutations at SA1, SA2 and SA3 |
| 7 | Modified HS4-650 insulator (r-c) − A to T mutation at SA2 |
| 8 | Modified HS4-650 insulator (r-c) − A to T mutations at SA2 and SA3 |
| 9 | Modified HS4-650 insulator (r-c) − A to T mutation at SA3 |
| 10 | Unmodified HS4-650 insulator (Genbank Acc. No. JN000001) |
| 11 | Unmodified HS4-650 insulator (Genbank Acc. No. JN000001) − reverse |
| | complement (r-c) |
| 12 | Modified HS4-650 insulator (Genbank Acc. No. JN000001) (r-c) A to T |
| | mutation at SA1 |
| 13 | Modified HS4-650 insulator (Genbank Acc. No. JN000001) (r-c) − A to T |
| | mutation at SA1 and SA2 |
| 14 | Modified HS4-650 insulator (Genbank Acc. No. JN000001) (r-c) − A to T |
| | mutation at SA1 and SA3 |
| 15 | Modified HS4-650 insulator (Genbank Acc. No. JN000001) (r-c) − A to T |
| | mutation at SA1, SA2 and SA3 |
| 16 | Modified HS4-650 insulator (Genbank Acc. No. JN000001) (r-c) − A to T |
| | mutation at SA2 |
| 17 | Modified HS4-650 insulator (Genbank Acc. No. JN000001) (r-c) − A to T |
| | mutation at SA2 and SA3 |
| 18 | Modified HS4-650 insulator (Genbank Acc. No. JN000001) (r-c) − A to T |
| | mutation at SA3 |
| 19 | Unmodified HS4-650 insulator (US2016003218) |
| 20 | Unmodified HS4-650 insulator (US2016003218) − reverse complement (r-c) |
| 21 | Modified HS4-650 insulator (US2016003218) (r-c) − A to T mutation at SA1 |
| 22 | Modified HS4-650 insulator (US2016003218) (r-c) − A to T mutation at SA1 |
| | and SA2 |
| 23 | Modified HS4-650 insulator (US2016003218) (r-c) − A to T mutation at SA1 |
| | and SA3 |
| 24 | Modified HS4-650 insulator (US2016003218) (r-c) − A to T mutation at SA1, |
| | SA2 and SA3 |
| 25 | Modified HS4-650 insulator (US2016003218) (r-c) − A to T mutation at SA2 |
| 26 | Modified HS4-650 insulator (US2016003218) (r-c) − A to T mutation at SA2 |
| | and SA3 |
| 27 | Modified HS4-650 insulator (US2016003218) (r-c) − A to T mutation at SA3 |
| 28 | Unmodified HS4-650 insulator (Genbank Acc. No. MN044710.1) |
| 29 | Unmodified HS4-650 insulator (Genbank Acc. No. MN044710.1) − reverse |
| | complement (r-c) |
| 30 | Modified HS4-650 insulator (Genbank Acc. No. MN044710.1) (r-c) − A to T |
| | mutation at SA1 |
| 31 | Modified HS4-650 insulator (Genbank Acc. No. MN044710.1) (r-c) − A to T |
| | mutation at SA1 and SA2 |
| 32 | Modified HS4-650 insulator (Genbank Acc. No. MN044710.1) (r-c) − A to T |
| | mutation at SA1 and SA3 |
| 33 | Modified HS4-650 insulator (Genbank Acc. No. MN044710.1) (r-c) − A to T |
| | mutation at SA1, SA2 and SA3 |
| 34 | Modified HS4-650 insulator (Genbank Acc. No. MN044710.1) (r-c) − A to T |
| | mutation at SA2 |
| 35 | Modified HS4-650 insulator (Genbank Acc. No. MN044710.1) (r-c) − A to T |
| | mutation at SA2 and SA3 |
| 36 | Modified HS4-650 insulator (Genbank Acc. No. MN044710.1) (r-c) − A to T |
| | mutation at SA3 |
| 37 | Unmodified HS4-650 insulator (Genbank Acc. No. MN044709) |
| 38 | Unmodified HS4-650 insulator (Genbank Acc. No. MN044709) − reverse |
| | complement (r-c) |
| 39 | Modified HS4-650 insulator (Genbank Acc. No. MN044709) (r-c) − A to T |
| | mutation at SA1 |
| 40 | Modified HS4-650 insulator (Genbank Acc. No. MN044709) (r-c) − A to T |
| | mutation at SA1 and SA2 |
| 41 | Modified HS4-650 insulator (Genbank Acc. No. MN044709) (r-c) − A to T |
| | mutation at SA1 and SA3 |
| 42 | Modified HS4-650 insulator (Genbank Acc. No. MN044709) (r-c) − A to T |
| | mutation at SA1, SA2 and SA3 |
| 43 | Modified HS4-650 insulator (Genbank Acc. No. MN044709) (r-c) − A to T |
| | mutation at SA2 |
| 44 | Modified HS4-650 insulator (Genbank Acc. No. MN044709) (r-c) − A to T |
| | mutation at SA2 and SA3 |
| 45 | Modified HS4-650 insulator (Genbank Acc. No. MN044709) (r-c) − A to T |
| | mutation at SA3 |
| 46 | Unmodified HS4-650 insulator (Genbank Acc. No. KF569217) |
| 47 | Unmodified HS4-650 insulator (Genbank Acc. No. KF569217) − reverse |
| | complement (r-c) |
| 48 | Modified HS4-650 insulator (Genbank Acc. No. KF569217) (r-c) − A to T |
| | mutation at SA1 |
| 49 | Modified HS4-650 insulator (Genbank Acc. No. KF569217) (r-c) − A to T |
| | mutation at SA1 and SA2 |
| 50 | Modified HS4-650 insulator (Genbank Acc. No. KF569217) (r-c) − A to T |
| | mutation at SA1 and SA3 |
| 51 | Modified HS4-650 insulator (Genbank Acc. No. KF569217) (r-c) − A to T |
| | mutation at SA1, SA2 and SA3 |
| 52 | Modified HS4-650 insulator (Genbank Acc. No. KF569217) (r-c) − A to T |
| | mutation at SA2 |
| 53 | Modified HS4-650 insulator (Genbank Acc. No. KF569217) (r-c) − A to T |
| | mutation at SA2 and SA3 |
| 54 | Modified HS4-650 insulator (Genbank Acc. No. KF569217) (r-c) − A to T |
| | mutation at SA3 |
| 55 | pBRNGTR47_pTL20c_SK734rev_MND_WAS_650 (“pBRNGTR47”) |
| 56 | pBRNGTR84_pTL20c_SK734rev_MND_WAS_650 (“pBRNGTR84”) |
| 57 | pBRNGTR88_pTL20c_SK734rev_MND_WAS_650_SAmut (“pBRNGTR88”) |
| 58 | pBRNGTR92_pTL20c_SK734rev_MND_WAS_650fwd (“pBRNGTR92”) |
| 59 | pBRNGTR120_pTL20c_SK734rev_MND_WAS_650_3xSAmut (“pBRNGTR120”) |
| 60 | SA1 sequence |
| 61 | SA2 sequence |
| 62 | SA3 sequence |
| 63 | Inactivated SA1 sequence |
| 64 | Inactivated SA2 sequence |
| 65 | Inactivated SA3 sequence |
| 66 | sh734 |
| 67 | sh734 with multi-t termination sequence |
| 68 | shRNA734 single t termination sequence |
| 69 | 7SK RNA promoter |
| 70 | 7SK RNA promoter |
| 71 | 7SK RNA promoter |
| 72 | MND promoter |
| 73 | WAS^{WT} cDNA (wild-type ORF) |
| 74 | WAS^{WT} cDNA (Genbank accession no. AB590224.1) |
| 75 | WAS^{WT} cDNA − codon optimised |
| 76 | WASP |
| 77 | WPRE mut6 |
| 78 | WPRE mut7 |
| 79 | 7tet operator |
| 80 | β-globin poly(A) signal |
| 81 | pBRNGTR83_pTL20c_MND_WAS_650 (“pBRNGTR83”) |
| 82 | pBRNGTR87_pTL20c_MND_WAS_650_SAmut (“pBRNGTR87”) |
| 83 | pBRNGTR91_pTL20c_MND_WAS_650fwd (“pBRNGTR91”) |
| 84 | pBRNGTR119_pTL20c_MND_WAS_650_3xSAmut (“pBRNGTR119”) |
| 85 | Unmodified γ-globin expression cassette |
| 86 | Unmodified γ-globin expression cassette − reverse complement (r-c) |
| 87 | Unmodified truncated HBB intron 2 |
| 88 | Unmodified truncated HBB intron 2 − reverse complement |
| 89 | Unmodified HS4-400 |
| 90 | Unmodified HS4-400 − reverse complement |
| 91 | Modified γ-globin expression cassette − reverse complement − G to A |
| | mutation at SD1 |
| 92 | Modified truncated HBB intron 3 |
| 93 | Modified HS4-400 − reverse complement − A to T mutation at SA2 |
| 94 | Modified HS4-400 − reverse complement − A to T mutation at SA2 and SA3 |
| 95 | Modified HS4-400 − reverse complement − A to T mutation at SA3 |
| 96 | SD1 sequence |
| 97 | Inactivated SD1 sequence |
| 98 | γ-globin Exon 1 |
| 99 | γ-globin Exon 2 |
| 100 | γ-globin Exon 3 |
| 101 | γ-globin coding sequence |
| 102 | γ-globin coding sequence |
| 103 | γ-globin G16D (GbGM^{G16D}) protein |
| 104 | β-globin poly(A) signal |
| 105 | β-globin Locus control region (LCR) |
| 106 | REV response element (RRE) |
| 107 | pCalH10_TL20c_rGbGM_7SKsh734 (“pCalH10”) |
| 108 | pCalH20_TL20d_rGbGM_7SKsh734 (“pCalH20”) |
| 109 | pCalH21_TL20d_rGbGM_G3320A_7SKsh734 (“pCalH21”) |
| 110 | pCalH32_TL20c_rGbGM_7SKsh734_400_2AT (“pCalH32”) |
| 111 | pCalH13_TL20c_rGbGM_G3320A_7SKsh734_400_2AT (“pCalH13”) |
| 112 | pCalH11_TL20c_rGbGM_G3320A_7SKsh734 (“pCalH11”) |
| 113 | pCalH31_TL20c_rGbGM_7SKsh734_400_1AT (“pCalH31”) |
| 114 | pCalH12_TL20c_rGbGM_G3320A_7SKsh734_400_1AT (“pCalH12”) |
| 115 | β-globin promoter |
| 116 | β-globin promoter |
| 117 | β-globin promoter |
| 118 | Unmodified γ-globin transgene |
| 119 | Unmodified γ-globin transgene − reverse complement |
| 120 | Unmodified γ-globin transgene − reverse complement − G to A mutation at SD1 |
| 121 | Unmodified γ-globin transgene + polyA signal |
| 122 | Unmodified γ-globin transgene + polyA signal − reverse complement |
| 123 | Unmodified γ-globin transgene + polyA signal − reverse complement − G to |
| | A mutation at SD1 |

### 2. Lentiviral Vectors

The present disclosure provides lentiviral vectors useful for gene therapy applications, such as for treating a disease or condition including WAS or SCD. In an aspect, the lentiviral vectors comprise a first promoter operably linked to a transgene (i.e. operably linked to a first nucleic acid sequence, wherein the first nucleic acid sequence encodes a protein or polynucleotide, such as a therapeutic protein or polynucleotide), and a modified HS4-650 insulator. The modified HS4-650 insulator comprises an inactivation of one or more cryptic splice acceptor sites that are present in an unmodified HS4-650 insulator, when the insulator is present in a vector. In another aspect, the lentiviral vectors comprise a first promoter operably linked to a transgene (i.e. operably linked to a first nucleic acid sequence, wherein the first nucleic acid sequence encodes a protein or polynucleotide, such as a therapeutic protein or polynucleotide), and a modified HS4-400 insulator. The modified HS4-400 insulator comprises an inactivation of one or more cryptic splice acceptor sites that are present in an unmodified HS4-400 insulator, when the insulator is present in a vector.

Accordingly, the lentiviral vectors of the present disclosure can be associated with reduced alternative splicing (e.g. of the transcript of the gene into which the lentiviral vector has integrated in the cell; of the lentiviral vector RNA; and/or the transcript of the transgene encoded by the lentiviral vector) when integrated into the genome of a cell compared to a lentiviral vector that contains an unmodified HS4-650 insulator or unmodified HS4-400 insulator, as described herein. In some examples, the level of alternative splicing is reduced by at least or about 20%, 30%, 40%, 50%, 60%, 70%, 80% or 90%.

For the purposes of the present disclosure, a lentiviral vector is a vector which comprises nucleic acid that includes at least one component part derivable from a lentivirus. That component part may be involved in the biological mechanisms by which the vector infects cells, expresses genes or is replicated. Thus, lentiviral vectors include nucleic acid molecules such as plasmids, and virus particles.

The basic structure of retrovirus and lentivirus genomes share many common features such as a 5′ LTR and a 3′ LTR, between or within which are located a packaging signal to enable the genome to be packaged, a primer binding site, integration sites to enable integration into a host cell genome and gag, pol and env genes encoding the packaging components, which are polypeptides required for the assembly of viral particles. Lentiviruses have additional features, such as the rev and rev response element (RRE) sequences, which enable the efficient export of RNA transcripts of the integrated provirus from the nucleus to the cytoplasm of an infected target cell. In the provirus, the viral genes are flanked at both ends by regions called long terminal repeats (LTRs). The LTRs are responsible for proviral integration, and transcription. LTRs also serve as enhancer-promoter sequences and can control the expression of the viral genes. The LTRs themselves are identical sequences that can be divided into three elements, which are called “U3,” “R” and “U5.” U3 is derived from the sequence unique to the 3′ end of the RNA, R is derived from a sequence repeated at both ends of the RNA, and U5 is derived from the sequence unique to the 5′ end of the RNA. The sizes of the three elements can vary considerably among different viruses.

In one embodiment, at least part of one or more protein coding regions essential for replication may be removed from the vector, which makes the vector replication-defective. Portions of the viral genome may also be replaced by a nucleic acid in order to generate a vector comprising the nucleic acid which is capable of transducing a target non-dividing host cell and/or integrating its genome into a host genome. In one embodiment, the lentiviral vectors are non-integrating vectors as described in U.S. patent application Ser. No. 12/138,993 (herein incorporated by reference).

The lentiviral vector may have a genome that has been manipulated to remove the non-essential elements and to retain the essential elements in order to provide the required functionality to infect, transduce and deliver a nucleotide sequence of interest to a target host cell (see, e.g., U.S. Pat. No. 6,669,936, incorporated by reference). In some embodiments, the genome is limited to sufficient lentiviral genetic information to allow packaging of an RNA genome, in the presence of packaging components, into a viral particle capable of infecting a target cell. Infection of the target cell may include reverse transcription and integration into the target cell genome. In some embodiments, the vector is incapable of independent replication to produce infectious lentiviral particles within the final target cell. In some embodiments, the lentiviral vector lacks a functional gag-pol and/or env gene and/or other genes essential for replication.

In some examples, the lentiviral vector is a self-inactivating vector. Self-inactivating vectors may be constructed by deleting the transcriptional enhancers or the enhancers and promoter in the U3 region of the 3′ LTR. After a round of vector reverse transcription and integration, these changes are copied into both the 5′ and the 3′ LTRs producing a transcriptionally inactive provirus (Yu et al. (1986), Proceedings Nat'l Acad. Sci. USA, 83:3194-98; Dougherty and Temin et al. (1987), Proceedings Nat'l Acad. Sci. USA, 84:1197-01; Hawley (1987), Proceedings Nat'l Acad. Sci. USA, 84:2406-10; Yee et al. (1994), Proceedings Nat'l Acad. Sci. USA, 91:9564-68). However, any promoter(s) internal to the LTRs in such vectors will still be transcriptionally active. This strategy has been employed to eliminate effects of the enhancers and promoters in the viral LTRs on transcription from internally placed genes. Such effects include increased transcription (Jolly et al. (1983), Nucleic Acids Research, 11:1855-72) or suppression of transcription (Emerman and Temin (1984), Cell, 39:449-67). This strategy can also be used to eliminate downstream transcription from the 3′ LTR into genomic DNA (Herman & Coffin (1987), Science, 236:845-48).

A plasmid vector used to produce the viral genome within a host cell/packaging cell will also include transcriptional regulatory control sequences operably linked to the lentiviral genome to direct transcription of the genome in a host cell/packaging cell. These regulatory sequences may be the natural sequences associated with the transcribed lentiviral sequence, i.e. the 5′ U3 region, or they may be a heterologous or modified promoter such as another viral promoter, for example the CMV promoter or the 7tetO promoter/operator. Some lentiviral genomes require additional sequences for efficient virus production. For example, in the case of HIV-based lentiviral vectors, the rev and RRE sequences are preferably included; however the requirement for rev and RRE may be reduced or eliminated by codon optimization (See U.S. patent application Ser. No. 12/587,236, incorporated by reference). Alternative sequences which perform the same function, as the rev/RRE system are also known. For example, a functional analogue of the revIRRE system is found in the Mason Pfizer monkey virus. This is known as the constitutive transport element (CTE) and comprises an RRE-type sequence in the genome which is believed to interact with a factor in the infected cell. The cellular factor can be thought of as a rev analogue. Thus, CTE may be used as an alternative to the reviRRE system. Any other functional equivalents which are known or become available may be relevant to the vectors of the present disclosure. For example, the Rex protein of HTLV-1 can functionally replace the Rev protein of HIV-1. It is also known that Rev and Rex have similar effects to IRE-BP.

In some embodiments, the expression vector comprises sequences from the 5′ and 3′ long terminal repeats (LTRs) of a lentivirus. In some embodiments, the vector comprises the R and U5 sequences from the 5′ LTR of a lentivirus and an inactivated or self-inactivating 3′ LTR from a lentivirus. In some embodiments, the LTR sequences are HIV LTR sequences.

In some embodiments, the lentiviral vectors contemplated herein may be integrative or non-integrating (also referred to as an integration defective lentivirus). As used herein, the term “integration defective lentivirus” or “IDLV” refers to a lentivirus having an integrase that lacks the capacity to integrate the viral genome into the genome of the host cells. In some applications, the use of by an integrating lentivirus vector may avoid potential insertional mutagenesis induced by an integrating lentivirus. Integration defective lentiviral vectors typically are generated by mutating the lentiviral integrase gene or by modifying the attachment sequences of the LTRs (see, e.g., Sarkis et al. (2008), Curr. Gene. Ther., 6: 430-437). Lentiviral integrase is coded for by the HIV-1 Pol region and the region cannot be deleted as it encodes other critical activities including reverse transcription, nuclear import, and viral particle assembly. Mutations in pol that alter the integrase protein fall into one of two classes: those which selectively affect only integrase activity (Class I); or those that have pleiotropic effects (Class II). Mutations throughout the N and C terminals and the catalytic core region of the integrase protein generate Class II mutations that affect multiple functions including particle formation and reverse transcription. Class I mutations limit their affect to the catalytic activities, DNA binding, linear episome processing and multimerization of integrase. The most common Class I mutation sites are a triad of residues at the catalytic core of integrase, including D64, D116, and E152. Each mutation has been shown to efficiently inhibit integration with a frequency of integration up to four logs below that of normal integrating vectors while maintaining transgene expression of the NILV. Another alternative method for inhibiting integration is to introduce mutations in the integrase DNA attachment site (LTR att sites) within a 12 base-pair region of the U3 region or within an 11 base-pair region of the U5 region at the terminal ends of the 5′ and 3′ LTRs, respectively. These sequences include the conserved terminal CA dinucleotide which is exposed following integrase-mediated end-processing. Single or double mutations at the conserved CA/TG dinucleotide result in up to a three to four log reduction in integration frequency; however, it retains all other necessary functions for efficient viral transduction.

### 2.1 Transgene

The transgene can be any gene that encodes a therapeutic expression product (e.g. protein or polynucleotide) that can correct a defect in a target cell (e.g. HSCs). Transgenes can include genomic sequences, cDNA sequences, and smaller engineered gene segments that express, or may be adapted to express, proteins, polypeptides, domains, fusion proteins, and mutants that maintain some or all of the therapeutic function of the full-length polypeptide encoded by the transgene.

In particular embodiments, the transgene encodes a Wiskott-Aldrich Syndrome (WAS) protein (WASP). Thus, in some embodiments, the lentiviral vectors of the present disclosure comprise a first nucleic acid sequence, wherein the first nucleic acid sequence encodes a WASP. Exemplary WASP include those comprising the amino acid sequence set forth in SEQ ID NO:76, and those having at least or about 85%, 86%, 87%, 88%, 89%, 90%, 91%, 92%, 93%, 94%, 95%, 96%, 98%, or 99% sequence identity to the WASP set forth in SEQ ID NOs: 76. In some embodiments, the nucleic acid sequence encoding a WASP comprises a sequence having at least 80%, 81%, 82%, 83%, 84%, 85%, 86%, 87%, 88%, 89%, 90%, 91%, 92%, 93%, 94%, 95%, 96%, 98%, or 99% sequence identity to nucleic acid sequence set forth in any one of SEQ ID NOS: 73-75. In other embodiments, the transgene is a globin transgene, for example, a γ-globin transgene.

In particular embodiments, the transgene encodes a globin transgene. Thus, in some embodiments, the lentiviral vectors of the present disclosure comprise a first nucleic acid sequence, wherein the first nucleic acid sequence encodes a globin transgene. Exemplary globin transgenes include those comprising the amino acid sequence set forth in SEQ ID NO:103, and those having at least or about 85%, 86%, 87%, 88%, 89%, 90%, 91%, 92%, 93%, 94%, 95%, 96%, 98%, or 99% sequence identity to the protein set forth in SEQ ID NO: 103. In some embodiments, the nucleic acid sequence encoding a globin protein comprises a sequence having at least 80%, 81%, 82%, 83%, 84%, 85%, 86%, 87%, 88%, 89%, 90%, 91%, 92%, 93%, 94%, 95%, 96%, 98%, or 99% sequence identity to nucleic acid sequence set forth in any one of SEQ ID NOS: 101-102.

### 2.2 HS4-650 Insulator

Some lentiviral vectors of the present disclosure comprise a modified HS4-650 insulator that has one or more inactivated or disrupted splice acceptor sites relative to an unmodified HS4-650 insulator.

Insulator elements have two important activities: an “enhancer blocking activity” where the insulator prevents interaction between enhancers and promoters, and “barrier activity” whereby the insulator prevents transgene silencing by chromatin condensation. The barrier activity can effectively increase transgene expression, while the enhancer blocking activity can prevent enhancers in the vector acting on normally inactive oncogene promoters when integrated nearby.

The most well-characterized insulator with barrier and enhancer blocking functions is a 1.2 kb fragment which contains hypersensitive site 4 from the chicken β-globin locus (cHS4). While this insulator is effective at increasing transgene expression and reducing unwanted promoter activity, it has been shown to reduce viral titres, thereby limiting large-scale virus production for clinical use. An alternative form, the 650 bp cHS4 insulator, which comprises a HS4-Core (250 bp) and a HS4-Ext (400 bp) and is referred to as HS4-650 (or cHS4-650) retains the barrier and enhancer blocking functions but does not impact viral production in the same manner as the 1.2 kb fragment (see e.g. Arumugam et al. (2009), PLoS ONE, 4(9):e6995; Wielgosz et al. (2015), Molecular Therapy—Methods & Clinical Development, 2, 14063). Another alternative form, the 400 bp cHS4 insulator, which comprises a HS4-Ext (400 bp) and is referred to as HS4-400 (or cHS4 400), also retains the barrier and enhancer blocking functions but does not impact viral production in the same manner as the 1.2 kb fragment.

As determined herein, cHS4 derived insulators, including HS4-650 and HS4-400 insulators, can comprise cryptic splice acceptor sites when present in a viral vector. These splice acceptor sites were identified in the HS4-650 insulator set forth in SEQ ID NO:1 when the insulator was present in a lentiviral vector in the reverse orientation, whereby the splice acceptor sites were in the positive strand of the vector. Thus, the splice acceptor sites were in the reverse complement sequence of SEQ ID NO:1. This reverse complement sequence is set forth as SEQ ID NO:2. The splice acceptor sites include splice acceptor site 1 (SA1), splice acceptor site 2 (SA2) and splice acceptor site 3 (SA3). Table 2 sets forth the sequence and position of these splice sites in the HS4-650 insulator set forth in SEQ ID NO:2.

TABLE 2
Splice sites in HS4-650 insulators

| | | | Position of |
|---|---|---|---|
| | | Position of | splice site |
| Splice | Sequence of splice site in vector | sequence in | (dinucleotide) |
| site | 5′ intron^{∧}exon 3′ | SEQ ID NO: 2 | (SEQ ID NO: 2) |
| SA1 | TTGCATCCAG^{∧}ACACCATCAA | 376-395 | 385-386 |
| | (SEQ ID NO: 60) | | (G^{∧}A) |
| SA2 | ATCCCCCCAG^{∧}GTGTCTGCAG | 437-456 | 446-447 |
| | (SEQ ID NO: 61) | | (G^{∧}G) |
| SA3 | GTGTCTGCAG^{∧}GCTCAAAGAG | 447-466 | 456-457 |
| | (SEQ ID NO: 62) | | (G^{∧}G) |

### 2.2.1 SA1

As shown in Table 2, SA1 is present at position 385-386 of SEQ ID NO:2 (i.e. splicing occurs between the G at position 385 and the A at position 386) and corresponding positions of other reverse complement HS4-650 insulator sequences, including those set forth in SEQ ID NOs:11, 20, 29, 38 and 47 (see FIG. 1). SA1 can also be defined as comprising the sequence TTGCATCCAG\hat{ }ACACCATCAA (SEQ ID NO:60), where \hat{ } represents the splice position.

The lentiviral vectors of the present disclosure comprise a modified HS4-650 insulator that, when present in the lentiviral vector, comprises an inactivated SA1 (relative to an unmodified HS4-650 insulator when present in the lentiviral vector). Thus, the modified HS4-650 insulators, when present in the vector, comprise a modification relative to an unmodified HS4-650 insulator, wherein the modification results in inactivation of SA1. Thus, a lentiviral vector comprising the modified HS4-650 insulator exhibits reduced splicing at position 385-386 when transduced into a cell compared to the splicing that occurs at position 385-386 in a lentiviral vector that comprises an unmodified HS4-650 insulator, with numbering relative to SEQ ID NO:2. In some examples, splicing is reduced by at least or about 20%, 30%, 40%, 50%, 60%, 70%, 80% or 90%. In some embodiments, the modification is or comprises a mutation in the sequence of the modified HS4-650 insulator relative to an unmodified HS4-650 insulator. In other examples, the modification is a change in the orientation of the modified HS4-650 insulator in the vector relative to the orientation of an unmodified HS4-650 insulator when in the vector. As would be appreciated, where the modification is a change in the orientation of the insulator, there may be no modification of the sequence of the modified HS4-650 insulator compared to an unmodified HS4-650 insulator.

Unmodified HS4-650 insulators include those that, when present in a lentiviral vector, comprise an active SA1, i.e. comprise a sequence and orientation within the lentiviral vector that can facilitate splicing at SA1. Exemplary unmodified HS4-650 insulators comprise a sequence set forth in SEQ ID NOs:1, 10, 19, 28, 37 and 46 (with reverse complement sequences set forth in SEQ ID NOs:2, 11, 20, 29, 38 and 47) and sequences having at least or about 90%, 91%, 92%, 93%, 94%, 95%, 96%, 97%, 98% or 99% sequence identity thereto (provided SA1 site is still present, e.g. provided the reverse complement of the HS4-650 insulator comprises the sequence TTGCATCCAGACACCATCAA (SEQ ID NO:60)). In some examples, an unmodified HS4-650 insulator is one in the reverse orientation in the lentiviral vector, such that SA1 is present on the positive strand. In further examples, an unmodified HS4-650 insulator is one in the reverse orientation compared to the transgene, such that SA1 is present on the positive strand of the transgene transcript.

In particular examples, the modified HS4-650 insulator contains a mutation (e.g. a nucleotide deletion, insertion or replacement) relative to an unmodified HS4-650 insulator, wherein the mutation inactivates SA1 that is present in the unmodified HS4-650 insulator (or reduces splicing at position 385-386 of the reverse complement sequence of the modified HS4-650 insulator compared to the splicing that occurs at position 385-386 of the reverse complement sequence of an unmodified HS4-650 insulator, with numbering relative to SEQ ID NO:2). The mutation can be any that inactivates or disrupts SA1. In some examples, the mutation is a deletion or substitution of any nucleotide in the SA1 sequence or a nucleotide insertion into the SA1 sequence (e.g. the sequence TTGCATCCAGACACCATCAA (SEQ ID NO:60)). In particular examples, the mutation is a mutation (e.g. deletion or substitution) of the A at position 384, the G at position 385, the A at position 386, and/or the C at position 387, with numbering relative to SEQ ID NO:2. For example, the modified HS4-650 insulator can comprise an A to T, A to C or A to G mutation at position 384, a G to C, G to A or G to T mutation at position 385, an A to T, A to C or A to G mutation at position 386, and/or a C to G, C to T or C to A mutation at position 387, with numbering relative to SEQ ID NO:2. In other examples, the mutation comprises an insertion of a nucleotide after position 384, 385 or 386. In some examples, the modified HS4-650 insulator comprises two or more of such mutations.

In one example, the modified HS4-650 insulator comprises an A to T mutation in the reverse complement sequence (i.e. in the complementary strand) at position 384, with numbering relative to SEQ ID NO:2. In particular embodiments, the reverse complement sequence of the modified HS4-650 insulator comprises the sequence set forth in any one of SEQ ID NOs:3, 12, 21, 30, 39 and 48 or a sequence having at least or about 90%, 91%, 92%, 93%, 94%, 95%, 96%, 97%, 98% or 99% sequence identity thereto (provided there is T position 384, with numbering relative to SEQ ID NO:2).

In some examples, the modified HS4-650 insulator described herein having a mutation that inactivates SA1 is in the opposite orientation to the transgene (i.e. in the opposite orientation to the first nucleic acid sequence). In particular examples, the first nucleic acid is in the forward orientation and the modified HS4-650 insulator is in the reverse orientation within the lentiviral vector.

In a further example, the modified HS4-650 insulator is in the lentiviral vector in the opposition orientation to an unmodified HS4-650 insulator when in the lentiviral vector, i.e. the orientation of the modified HS4-650 insulator inverted relative to an unmodified HS4-650 insulator, so as to inactivate SA1. In particular examples, the modified HS4-650 insulator is in the forward orientation in the vector. Thus, also provided are lentiviral vectors comprising a first promoter operably linked to a first nucleic acid sequence, wherein the first nucleic acid sequence encodes a WASP; and a HS4-650 insulator, wherein the HS4-650 insulator is in the forward orientation in the vector. In some examples, the first nucleic acid sequence is also in the forward orientation in the vector. In some examples, the HS4-650 insulator comprises a sequence set forth in any one of SEQ ID NOs:1, 10, 19, 28, 37 and 46 or a sequence having at least or about 90%, 91%, 92%, 93%, 94%, 95%, 96%, 97%, 98% or 99% sequence identity thereto.

### 2.2.2 SA2

SA2 is present at position 446-447 of SEQ ID NO:2 (i.e. splicing occurs between the G at position 446 and the G at position 447) and corresponding positions of other reverse complement HS4-650 insulator sequences, including those set forth in SEQ ID NOs:11, 20, 29, 38 and 47 (see FIG. 1). SA2 can also be defined as comprising the sequence ATCCCCCCAG\hat{ }GTGTCTGCAG (SEQ ID NO:61), where \hat{ } represents the splice position.

The lentiviral vectors of the present disclosure comprise a modified HS4-650 insulator that, when present in the lentiviral vector, comprises an inactivated SA2 (relative to an unmodified HS4-650 insulator when present in the lentiviral vector). Thus, the modified HS4-650 insulators, when present in the vector, comprise a modification relative to an unmodified HS4-650 insulator, wherein the modification results in inactivation of SA2. Thus, a lentiviral vector comprising the modified HS4-650 insulator exhibits reduced splicing at position 446-447 when transduced into a cell compared to the splicing that occurs at position 446-447 with a lentiviral vector that comprises an unmodified HS4-650 insulator, with numbering relative to SEQ ID NO:2. In some examples, splicing is reduced by at least or about 20%, 30%, 40%, 50%, 60%, 70%, 80% or 90%. In some embodiments, the modification is or comprises a mutation in the sequence of the modified HS4-650 insulator relative to an unmodified HS4-650 insulator. In other examples, the modification is a change in the orientation of the modified HS4-650 insulator in the vector relative to the orientation of an unmodified HS4-650 insulator when in the vector. As would be appreciated, where the modification is a change in the orientation of the insulator, there may be no modification of the sequence of the modified HS4-650 insulator compared to an unmodified HS4-650 insulator.

Unmodified HS4-650 insulators include those that, when present in a lentiviral vector, comprise an active SA2, i.e. comprise a sequence and orientation within the lentiviral vector that can facilitate splicing at SA2. Exemplary unmodified HS4-650 insulators comprise a sequence set forth in SEQ ID NOs:1, 10, 19, 28, 37 and 46 (with reverse complement sequences set forth in SEQ ID NOs:2, 11, 20, 29, 38 and 47) and sequences having at least or about 90%, 91%, 92%, 93%, 94%, 95%, 96%, 97%, 98% or 99% sequence identity thereto (provided the SA2 site is still present, e.g. provided the reverse complement of the HS4-650 insulator comprises the sequence ATCCCCCCAGGTGTCTGCAG (SEQ ID NO:61)). In some examples, an unmodified HS4-650 insulator is one in the reverse orientation in the lentiviral vector, such that SA2 is present on the positive strand. In further examples, an unmodified HS4-650 insulator is one in the reverse orientation compared to the transgene, such that SA2 is present on the positive strand of the transgene transcript.

In particular examples, the modified HS4-650 insulator contains a mutation (e.g. a nucleotide deletion, insertion or replacement) relative to an unmodified HS4-650 insulator, wherein the mutation inactivates SA2 that is present in the unmodified HS4-650 insulator (or reduces splicing at position 446-447 of the reverse complement sequence of the modified HS4-650 insulator compared to the splicing that occurs at position 446-447 of the reverse complement sequence of an unmodified HS4-650 insulator, with numbering relative to SEQ ID NO:2). The mutation can be any that inactivates or disrupts SA2. In some examples, the mutation is a deletion or substitution of any nucleotide in the SA2 sequence or a nucleotide insertion into the SA2 sequence (e.g. the sequence ATCCCCCCAGGTGTCTGCAG (SEQ ID NO:61)). In particular examples, the mutation is a mutation (e.g. deletion or substitution) of the A at position 445, the G at position 446, the G at position 447, and/or the T a position 448, with numbering relative to SEQ ID NO:2. For example, the modified HS4-650 insulator can comprise an A to T, A to C or A to G mutation at position 445, a G to C, G to A or G to T mutation at position 446, an G to C, G to T or G to A mutation at position 447, and/or a T to A, T to C or T to G mutation at position 448, with numbering relative to SEQ ID NO:2. In other examples, the mutation comprises an insertion of a nucleotide after position 445, 446 or 447. In some examples, the modified HS4-650 insulator comprises two or more of such mutations.

In one example, the modified HS4-650 insulator comprises an A to T mutation in the reverse complement sequence at position 445, with numbering relative to SEQ ID NO:2. In particular embodiments, the reverse complement sequence of the modified HS4-650 insulator comprises the sequence set forth in any one of SEQ ID NOs:7, 16, 25, 34, 43 and 52 or a sequence having at least or about 90%, 91%, 92%, 93%, 94%, 95%, 96%, 97%, 98% or 99% sequence identity thereto (provided there is T at position 445, with numbering relative to SEQ ID NO:2).

In some examples, the modified HS4-650 insulator described herein having a mutation that inactivates SA2 is in the opposite orientation to the transgene (i.e. in the opposite orientation to the first nucleic acid sequence). In particular examples, the first nucleic acid is in the forward orientation and the modified HS4-650 insulator is in the reverse orientation within the lentiviral vector.

In a further example, the modified HS4-650 insulator is in the lentiviral vector in the opposition orientation to an unmodified HS4-650 insulator when in the lentiviral vector, i.e. the orientation of the modified HS4-650 insulator inverted relative to an unmodified HS4-650 insulator, so as to inactivate SA2. In particular examples, the modified HS4-650 insulator is in the forward orientation in the vector.

### 2.2.3 SA3

SA3 is present at position 456-457 of SEQ ID NO:2 (i.e. splicing occurs between the G at position 456 and the G at position 457) and corresponding positions of other reverse complement HS4-650 insulator sequences, including those set forth in SEQ ID NOs:11, 20, 29, 38 and 47 (see FIG. 1). SA3 can also be defined as comprising the sequence GTGTCTGCAG\hat{ }GCTCAAAGAG (SEQ ID NO:62), where \hat{ } represents the splice position.

The lentiviral vectors of the present disclosure comprise a modified HS4-650 insulator that, when present in the lentiviral vector, comprises an inactivated SA3 (relative to an unmodified HS4-650 insulator when present in the lentiviral vector). Thus, the modified HS4-650 insulators, when present in the vector, comprise a modification relative to an unmodified HS4-650 insulator, wherein the modification results in inactivation of SA3. Thus, a lentiviral vector comprising the modified HS4-650 insulator exhibits reduced splicing at position 456-457 when transduced into a cell compared to the splicing that occurs at position 456-457 with a lentiviral vector that comprises an unmodified HS4-650 insulator, with numbering relative to SEQ ID NO:2. In some examples, splicing is reduced by at least or about 20%, 30%, 40%, 50%, 60%, 70%, 80% or 90%. In some embodiments, the modification is or comprises a mutation in the sequence of the modified HS4-650 insulator relative to an unmodified HS4-650 insulator. In other examples, the modification is a change in the orientation of the modified HS4-650 insulator in the vector relative to the orientation of an unmodified HS4-650 insulator when in the vector. As would be appreciated, where the modification is a change in the orientation of the insulator, there may be no modification of the sequence of the modified HS4-650 insulator compared to an unmodified HS4-650 insulator.

Unmodified HS4-650 insulators include those that, when present in a lentiviral vector, comprise an active SA3, i.e. comprise a sequence and orientation within the lentiviral vector that can facilitate splicing at SA3. Exemplary unmodified HS4-650 insulators comprise a sequence set forth in SEQ ID NOs:1, 10, 19, 28, 37 and 46 (with reverse complement sequences set forth in SEQ ID NOs:2, 11, 20, 29, 38 and 47) and sequences having at least or about 90%, 91%, 92%, 93%, 94%, 95%, 96%, 97%, 98% or 99% sequence identity thereto (provided the SA3 site is still present, e.g. provided the reverse complement of the HS4-650 insulator comprises the sequence GTGTCTGCAGGCTCAAAGAG (SEQ ID NO:62)). In some examples, an unmodified HS4-650 insulator is one in the reverse orientation in the lentiviral vector, such that SA3 is present on the positive strand. In further examples, an unmodified HS4-650 insulator is one in the reverse orientation compared to the transgene, such that SA3 is present on the positive strand of the transgene transcript.

In particular examples, the modified HS4-650 insulator contains a mutation (e.g. a nucleotide deletion, insertion or replacement) relative to an unmodified HS4-650 insulator, wherein the mutation inactivates SA3 that is present in the unmodified HS4-650 insulator (or reduces splicing at position 456-457 of the reverse complement sequence of the modified HS4-650 insulator compared to the splicing that occurs at position 456-457 of the reverse complement sequence of an unmodified HS4-650 insulator, with numbering relative to SEQ ID NO:2). The mutation can be any that inactivates or disrupts SA3. In some examples, the mutation is a deletion or substitution of any nucleotide in the SA3 sequence or a nucleotide insertion into the SA3 sequence (e.g. the sequence GTGTCTGCAGGCTCAAAGAG (SEQ ID NO:62)). In particular examples, the mutation is a mutation (e.g. deletion or substitution) of the A at position 455, the G at position 446, the G at position 457, and/or the C a position 458, with numbering relative to SEQ ID NO:2. For example, the modified HS4-650 insulator can comprise an A to T, A to C or A to G mutation at position 455, a G to C, G to A or G to T mutation at position 456, an G to C, G to T or G to A mutation at position 447, and/or a C to A, C to G or C to T mutation at position 458, with numbering relative to SEQ ID NO:2. In other examples, the mutation comprises an insertion of a nucleotide after position 455, 456 or 457. In some examples, the modified HS4-650 insulator comprises two or more of such mutations.

In one example, the modified HS4-650 insulator comprises an A to T mutation in the reverse complement sequence at position 455, with numbering relative to SEQ ID NO:2. In particular embodiments, the reverse complement sequence of the modified HS4-650 insulator comprises the sequence set forth in any one of SEQ ID NOs: 9, 18, 27, 36, 45 and 54 or a sequence having at least or about 90%, 91%, 92%, 93%, 94%, 95%, 96%, 97%, 98% or 99% sequence identity thereto (provided there is T at position 455, with numbering relative to SEQ ID NO:2).

In some examples, the modified HS4-650 insulator described herein having a mutation that inactivates SA3 is in the opposite orientation to the transgene (i.e. in the opposite orientation to the first nucleic acid sequence). In particular examples, the first nucleic acid is in the forward orientation and the modified HS4-650 insulator is in the reverse orientation within the lentiviral vector.

In a further example, the modified HS4-650 insulator is in the lentiviral vector in the opposition orientation to an unmodified HS4-650 insulator when in the lentiviral vector, i.e. the orientation of the modified HS4-650 insulator inverted relative to an unmodified HS4-650 insulator, so as to inactivate SA3. In particular examples, the modified HS4-650 insulator is in the forward orientation in the vector.

### 2.2.4 Combination Mutations

Modified HS4-650 insulators can comprise two or mutations that inactivate two or more of SA1, SA2 or SA3, relative to an unmodified HS4-650 insulator. Any of the mutations described above for inactivating SA1, SA2 and/or SA3 can be combined in a modified HS4-650 insulator.

In one example, the modified HS4-650 insulator comprises a mutation that inactivates SA1 and a mutation that inactivates SA2. For example, the modified HS4-650 insulator can comprise an A to T mutation in the reverse complement sequence at position 384, with numbering relative to SEQ ID NO:2, and an A to T mutation in the reverse complement sequence at position 445, with numbering relative to SEQ ID NO:2. In particular embodiments, the reverse complement sequence of the modified HS4-650 insulator comprises the sequence set forth in any one of SEQ ID NOs: 4, 13, 22, 31, 40 and 49 or a sequence having at least or about 90%, 91%, 92%, 93%, 94%, 95%, 96%, 97%, 98% or 99% sequence identity thereto (provided there is a T at position 384 and a T at position 445, with numbering relative to SEQ ID NO:2).

In another example, the modified HS4-650 insulator comprises a mutation that inactivates SA1 and a mutation that inactivates SA3. For example, the modified HS4-650 insulator can comprise an A to T mutation in the reverse complement sequence at position 384, with numbering relative to SEQ ID NO:2, and an A to T mutation in the reverse complement sequence at position 455, with numbering relative to SEQ ID NO:2. In particular embodiments, the reverse complement sequence of the modified HS4-650 insulator comprises the sequence set forth in any one of SEQ ID NOs: 5, 14, 23, 32, 41 and 50 or a sequence having at least or about 90%, 91%, 92%, 93%, 94%, 95%, 96%, 97%, 98% or 99% sequence identity thereto (provided there is a T at position 384 and a T at position 455, with numbering relative to SEQ ID NO:2).

The modified HS4-650 insulator may also comprise a mutation that inactivates SA2 and a mutation that inactivates SA3. For example, the modified HS4-650 insulator can comprise an A to T mutation in the reverse complement sequence at position 445, with numbering relative to SEQ ID NO:2, and an A to T mutation in the reverse complement sequence at position 455, with numbering relative to SEQ ID NO:2. In particular embodiments, the reverse complement sequence of the modified HS4-650 insulator comprises the sequence set forth in any one of SEQ ID NOs: 8, 17, 26, 35, 44 and 43 or a sequence having at least or about 90%, 91%, 92%, 93%, 94%, 95%, 96%, 97%, 98% or 99% sequence identity thereto (provided there is a T at position 445 and a T mutation at position 455, with numbering relative to SEQ ID NO:2).

In another example, the modified HS4-650 insulator comprises a mutation that inactivates SA1, a mutation that inactivates SA2 and a mutation that inactivates SA3. For example, in some embodiments, the modified HS4-650 insulator comprises an A to T mutation in the reverse complement sequence at position 384, an A to T mutation in the reverse complement sequence at position 445, and an A to T mutation in the reverse complement sequence at position 455, with numbering relative to SEQ ID NO:2. In particular embodiments, the reverse complement sequence of the modified HS4-650 insulator comprises the sequence set forth in any one of SEQ ID NOs: 6, 15, 24, 33, 42 and 51 or a sequence having at least or about 90%, 91%, 92%, 93%, 94%, 95%, 96%, 97%, 98% or 99% sequence identity thereto (provided there a T at position 384, a T at position 445 and a T at position 455, with numbering relative to SEQ ID NO:2).

### 2.3 HS4-400 Insulator

The lentiviral vectors of the present disclosure can comprise a HS4-400 insulator. In particular embodiments, the HS4-400 insulator is a modified HS4-400 insulator that has one or more inactivated or disrupted splice acceptor sites relative to an unmodified HS4-400 insulator. An exemplary HS4-400 insulator is one comprising a sequence set forth in SEQ ID NO:89 or one having at least or about 85%, 86%, 87%, 88%, 89%, 90%, 91%, 92%, 93%, 94%, 95%, 96%, 98%, or 99% sequence identity thereto.

As determined herein, HS4-400 insulators can comprise cryptic splice acceptor sites when present in a viral vector. These splice acceptor sites were identified in the HS4-400 insulator set forth in SEQ ID NO:89 when the insulator was present in a lentiviral vector in the reverse orientation, whereby the splice acceptor sites were in the positive strand of the vector. Thus, the splice acceptor sites were in the reverse complement sequence of SEQ ID NO:89. This reverse complement sequence is set forth as SEQ ID NO:90. The splice acceptor sites include splice acceptor site 2 (SA2) and splice acceptor site 3 (SA3). Table 3 sets forth the sequence and position of these splice sites in the HS4-400 insulator sequence set forth in SEQ ID NO:90.

TABLE 3
Splice sites in HS4-400 insulators

| | | | Position of |
|---|---|---|---|
| | | | splice site |
| | | Position of | (dinucleotide) |
| Splice | Sequence of splice site in vector | sequence in | in |
| site | 5′ intron^{∧}exon 3′ | SEQ ID NO: 90 | SEQ ID NO: 90 |
| SA2 | ATCCCCCCAG^{∧}GTGTCTGCAG | 181-200 | 190-191 |
| | (SEQ ID NO: 61) | | (G^{∧}G) |
| SA3 | GTGTCTGCAG^{∧}GCTCAAAGAG | 191-210 | 200-201 |
| | (SEQ ID NO: 62) | | (G^{∧}G) |

### 2.3.1 SA2

SA2 is present at position 190-191 of SEQ ID NO:90 (i.e. splicing occurs between the G at position 190 and the G at position 191) and corresponding positions of other reverse complement HS4-400 insulator sequences. SA2 can also be defined as comprising the sequence ATCCCCCCAG\hat{ }GTGTCTGCAG (SEQ ID NO:61), where \hat{ } represents the splice position, or comprising the sequence of nucleotides at positions 181-200 of the complementary strand of an HS4-400 insulator, with numbering relative to SEQ ID NO:90.

The lentiviral vectors of the present disclosure comprise a modified HS4-400 insulator that, when present in the lentiviral vector, comprises an inactivated SA2 (relative to an unmodified HS4-400 insulator when present in the lentiviral vector). Thus, the modified HS4-400 insulators, when present in the vector, comprise a modification relative to an unmodified HS4-400 insulator, wherein the modification results in inactivation of SA2. Thus, a lentiviral vector comprising the modified HS4-400 insulator exhibits reduced splicing at position 190-191 when transduced into a cell compared to the splicing that occurs at position 190-191 with a lentiviral vector that comprises an unmodified HS4-400 insulator, with numbering relative to SEQ ID NO:90. In some examples, splicing is reduced by at least or about 20%, 30%, 40%, 50%, 60%, 70%, 80% or 90%. In some embodiments, the modification is or comprises a mutation in the sequence of the modified HS4-400 insulator relative to an unmodified HS4-400 insulator. In other examples, the modification is a change in the orientation of the modified HS4-400 insulator in the vector relative to the orientation of an unmodified HS4-400 insulator when in the vector. As would be appreciated, where the modification is a change in the orientation of the insulator, there may be no modification of the sequence of the modified HS4-400 insulator compared to an unmodified HS4-400 insulator.

Unmodified HS4-400 insulators include those that, when present in a lentiviral vector, comprise an active SA2, i.e. comprise a sequence and orientation within the lentiviral vector that can facilitate splicing at SA2. Exemplary unmodified HS4-400 insulators include those that comprise a sequence set forth in SEQ ID NO:89 (with reverse complement sequences set forth in SEQ ID NO:90) and sequences having at least or about 90%, 91%, 92%, 93%, 94%, 95%, 96%, 97%, 98% or 99% sequence identity thereto (provided the SA2 site is still present, e.g. provided the reverse complement of the HS4-400 insulator comprises the sequence ATCCCCCCAGGTGTCTGCAG (SEQ ID NO:61)). In some examples, an unmodified HS4-400 insulator is one in the reverse orientation in the lentiviral vector, such that SA2 is present on the positive strand.

In particular examples, the modified HS4-400 insulator contains a mutation (e.g. a nucleotide deletion, insertion or replacement) relative to an unmodified HS4-400 insulator, wherein the mutation inactivates SA2 that is present in the unmodified HS4-400 insulator (or reduces splicing at position 190-191 of the reverse complement sequence of the modified HS4-400 insulator compared to the splicing that occurs at position 190-191 of the reverse complement sequence of an unmodified HS4-400 insulator, with numbering relative to SEQ ID NO:90). The mutation can be any that inactivates or disrupts SA2. In some examples, the mutation is a deletion or substitution of any nucleotide in the SA2 sequence or a nucleotide insertion into the SA2 sequence (e.g. the sequence ATCCCCCCAGGTGTCTGCAG (SEQ ID NO:61)). In particular examples, the mutation is a mutation (e.g. deletion or substitution) of the A at position 189, the G at position 190, the G at position 191, and/or the T a position 192, with numbering relative to SEQ ID NO:90. For example, the modified HS4-400 insulator can comprise an A to T, A to C or A to G mutation at position 189, a G to C, G to A or G to T mutation at position 190, an G to C, G to T or G to A mutation at position 191, and/or a T to A, T to C or T to G mutation at position 192, with numbering relative to SEQ ID NO:90. In other examples, the mutation comprises an insertion of a nucleotide after position 189, 190 or 191. In some examples, the modified HS4-400 insulator comprises two or more of such mutations.

In one example, the modified HS4-400 insulator comprises an A to T mutation in the reverse complement sequence at position 189, with numbering relative to SEQ ID NO:90. In particular embodiments, the reverse complement sequence of the modified HS4-400 insulator comprises the sequence set forth in SEQ ID NO:93 or a sequence having at least or about 90%, 91%, 92%, 93%, 94%, 95%, 96%, 97%, 98% or 99% sequence identity thereto (provided there is a T at position 189, with numbering relative to SEQ ID NO:90).

In some examples, the modified HS4-400 insulator described herein having a mutation that inactivates SA2 is in the reverse orientation within the lentiviral vector.

In a further example, the modified HS4-400 insulator is in the lentiviral vector in the opposition orientation to an unmodified HS4-400 insulator when in the lentiviral vector, i.e. the orientation of the modified HS4-400 insulator is inverted relative to an unmodified HS4-400 insulator, so as to inactivate SA2. In particular examples, the modified HS4-400 insulator is in the forward orientation in the vector. Thus, also provided are lentiviral vectors comprising a first promoter operably linked to a first nucleic acid sequence, wherein the first nucleic acid sequence comprises a modified γ-globin transgene comprising a HBB intron 2; and a HS4-400 insulator, wherein the HS4-400 insulator is in the forward orientation in the vector. In some examples, the first nucleic acid sequence is in the reverse orientation in the vector. In some examples, the HS4-400 insulator comprises a sequence set forth in SEQ ID NO:90 or a sequence having at least or about 90%, 91%, 92%, 93%, 94%, 95%, 96%, 97%, 98% or 99% sequence identity thereto.

### 2.3.2 SA3

SA3 is present at position 200-201 of SEQ ID NO:90 (i.e. splicing occurs between the G at position 200 and the G at position 201) and corresponding positions of other reverse complement HS4-400 insulator sequences. SA3 can also be defined as comprising the sequence GTGTCTGCAG\hat{ }GCTCAAAGAG (SEQ ID NO:62), where \hat{ } represents the splice position, or comprising the sequence of nucleotides at positions 191-210 of the complementary strand of an HS4-400 insulator, with numbering relative to SEQ ID NO:90.

The lentiviral vectors of the present disclosure comprise a modified HS4-400 insulator that, when present in the lentiviral vector, comprises an inactivated SA3 (relative to an unmodified HS4-400 insulator when present in the lentiviral vector). Thus, the modified HS4-400 insulators, when present in the vector, comprise a modification relative to an unmodified HS4-400 insulator, wherein the modification results in inactivation of SA3. Thus, a lentiviral vector comprising the modified HS4-400 insulator exhibits reduced splicing at position 200-201 when transduced into a cell compared to the splicing that occurs at position 200-201 with a lentiviral vector that comprises an unmodified HS4-400 insulator, with numbering relative to SEQ ID NO:90. In some examples, splicing is reduced by at least or about 20%, 30%, 40%, 50%, 60%, 70%, 80% or 90%. In some embodiments, the modification is or comprises a mutation in the sequence of the modified HS4-400 insulator relative to an unmodified HS4-400 insulator. In other examples, the modification is a change in the orientation of the modified HS4-400 insulator in the vector relative to the orientation of an unmodified HS4-400 insulator when in the vector. As would be appreciated, where the modification is a change in the orientation of the insulator, there may be no modification of the sequence of the modified HS4-400 insulator compared to an unmodified HS4-400 insulator.

Unmodified HS4-400 insulators include those that, when present in a lentiviral vector, comprise an active SA3, i.e. comprise a sequence and orientation within the lentiviral vector that can facilitate splicing at SA3. Exemplary unmodified HS4-400 insulators include those that comprise a sequence set forth in SEQ ID NO:90 (with reverse complement sequences set forth in SEQ ID NO:89) and sequences having at least or about 90%, 91%, 92%, 93%, 94%, 95%, 96%, 97%, 98% or 99% sequence identity thereto (provided the SA3 site is still present, e.g. provided the reverse complement of the HS4-400 insulator comprises the sequence GTGTCTGCAGGCTCAAAGAG (SEQ ID NO:62)). In some examples, an unmodified HS4-400 insulator is one in the reverse orientation in the lentiviral vector, such that SA3 is present on the positive strand.

In particular examples, the modified HS4-400 insulator contains a mutation (e.g. a nucleotide deletion, insertion or replacement) relative to an unmodified HS4-400 insulator, wherein the mutation inactivates SA3 that is present in the unmodified HS4-400 insulator (or reduces splicing at position 200-201 of the reverse complement sequence of the modified HS4-400 insulator compared to the splicing that occurs at position 200-201 of the reverse complement sequence of an unmodified HS4-400 insulator, with numbering relative to SEQ ID NO:90). The mutation can be any that inactivates or disrupts SA3. In some examples, the mutation is a deletion or substitution of any nucleotide in the SA3 sequence or a nucleotide insertion into the SA3 sequence (e.g. the sequence GTGTCTGCAGGCTCAAAGAG (SEQ ID NO:62)). In particular examples, the mutation is a mutation (e.g. deletion or substitution) of the A at position 199, the G at position 200, the G at position 201, and/or the C a position 202, with numbering relative to SEQ ID NO:90. For example, the modified HS4-400 insulator can comprise an A to T, A to C or A to G mutation at position 199, a G to C, G to A or G to T mutation at position 200, an G to C, G to T or G to A mutation at position 201, and/or a C to A, C to G or C to T mutation at position 202, with numbering relative to SEQ ID NO:90. In other examples, the mutation comprises an insertion of a nucleotide after position 199, 200 or 201. In some examples, the modified HS4-400 insulator comprises two or more of such mutations.

In one example, the modified HS4-400 insulator comprises an A to T mutation in the reverse complement sequence at position 199, with numbering relative to SEQ ID NO:90. In particular embodiments, the reverse complement sequence of the modified HS4-400 insulator comprises the sequence set forth in any one of SEQ ID NO:95 or a sequence having at least or about 90%, 91%, 92%, 93%, 94%, 95%, 96%, 97%, 98% or 99% sequence identity thereto (provided there is an A to T mutation position 199, with numbering relative to SEQ ID NO:90).

In some examples, the modified HS4-400 insulator described herein having a mutation that inactivates SA3 is in the reverse orientation within the lentiviral vector.

In a further example, the modified HS4-400 insulator is in the lentiviral vector in the opposition orientation to an unmodified HS4-400 insulator when in the lentiviral vector, i.e. the orientation of the modified HS4-400 insulator inverted relative to an unmodified HS4-400 insulator, so as to inactivate SA3. In particular examples, the modified HS4-400 insulator is in the forward orientation in the vector.

### 2.3.3 Combination Mutations

Modified HS4-400 insulators can comprise two or more mutations that inactivate both SA2 and SA3, relative to an unmodified HS4-400 insulator. Any of the mutations described above for inactivating SA2 or SA3 can be combined in a modified HS4-400 insulator.

In one example, the modified HS4-400 insulator comprises an A to T mutation in the reverse complement sequence at position 189 (i.e. comprises a T at position 189), with numbering relative to SEQ ID NO:90 and an A to T mutation in the reverse complement sequence at position 199 (i.e. comprises a T a position 199), with numbering relative to SEQ ID NO:90. In particular embodiments, the reverse complement sequence of the modified HS4-400 insulator comprises the sequence set forth in SEQ ID NO:94 or a sequence having at least or about 90%, 91%, 92%, 93%, 94%, 95%, 96%, 97%, 98% or 99% sequence identity thereto (provided there is a T at position 189 and T mutation at position 199, with numbering relative to SEQ ID NO:90).

### 2.4 Components to Inhibit Expression of the HPRT Gene

In some embodiments, the lentiviral vectors of the present disclosure comprise a nucleic acid sequence that encodes an agent that inhibits HPRT expression. Thus in some examples, the lentiviral vectors comprise a second promoter operably linked to a second nucleic acid sequence, wherein the second nucleic acid sequence encodes a nucleic acid that inhibits HPRT expression. In some embodiments, the RNAi agent is an shRNA, a microRNA, or a hybrid thereof.

### 2.4.1 RNAi

In some embodiments, the expression vector comprises a second nucleic acid sequence encoding an RNAi. RNA interference is an approach for post-transcriptional silencing of gene expression by triggering degradation of homologous transcripts through a complex multistep enzymatic process, e.g. a process involving sequence-specific double-stranded small interfering RNA (siRNA). A simplified model for the RNAi pathway is based on two steps, each involving a ribonuclease enzyme. In the first step, the trigger RNA (either dsRNA or miRNA primary transcript) is processed into a short, interfering RNA (siRNA) by the RNase II enzymes DICER and Drosha. In the second step, siRNAs are loaded into the effector complex RNA-induced silencing complex (RISC). The siRNA is unwound during RISC assembly and the single-stranded RNA hybridizes with mRNA target. It is believed that gene silencing is a result of nucleolytic degradation of the targeted mRNA by the RNase H enzyme Argonaute (Slicer). If the siRNA/mRNA duplex contains mismatches the mRNA is not cleaved. Rather, gene silencing is a result of translational inhibition.

In some embodiments, the RNAi agent is an inhibitory or silencing nucleic acid. As used herein, a “silencing nucleic acid” refers to any polynucleotide which is capable of interacting with a specific sequence to inhibit gene expression. Examples of silencing nucleic acids include RNA duplexes (e.g. siRNA, shRNA), locked nucleic acids (“LNAs”), antisense RNA, DNA polynucleotides which encode sense and/or antisense sequences of the siRNA or shRNA, DNAzymses, or ribozymes. The skilled artisan will appreciate that the inhibition of gene expression need not necessarily be gene expression from a specific enumerated sequence, and may be, for example, gene expression from a sequence controlled by that specific sequence.

Methods for constructing interfering RNAs are known in the art. For example, the interfering RNA can be assembled from two separate oligonucleotides, where one strand is the sense strand and the other is the antisense strand, wherein the antisense and sense strands are self-complementary (i.e., each strand comprises nucleotide sequence that is complementary to nucleotide sequence in the other strand; such as where the antisense strand and sense strand form a duplex or double stranded structure); the antisense strand comprises nucleotide sequence that is complementary to a nucleotide sequence in a target nucleic acid molecule or a portion thereof (i.e., an undesired gene) and the sense strand comprises nucleotide sequence corresponding to the target nucleic acid sequence or a portion thereof. Alternatively, interfering RNA may be assembled from a single oligonucleotide, where the self-complementary sense and antisense regions are linked by means of nucleic acid based or non-nucleic acid-based linker(s). The interfering RNA can be a polynucleotide with a duplex, asymmetric duplex, hairpin or asymmetric hairpin secondary structure, having self-complementary sense and antisense regions, wherein the antisense region comprises a nucleotide sequence that is complementary to nucleotide sequence in a separate target nucleic acid molecule or a portion thereof and the sense region having nucleotide sequence corresponding to the target nucleic acid sequence or a portion thereof. The interfering RNA can be a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof, and wherein the circular polynucleotide can be processed either in vivo or in vitro to generate an active siRNA molecule capable of mediating RNA interference.

In some embodiments, the interfering RNA coding region encodes a self-complementary RNA molecule having a sense region, an antisense region and a loop region. When expressed, such an RNA molecule desirably forms a “hairpin” structure and is referred to herein as an “shRNA.” In some embodiments, the loop region is generally between about 2 and about 10 nucleotides in length. In other embodiments, the loop region is from about 6 to about 9 nucleotides in length. In some embodiments, the sense region and the antisense region are between about 15 and about 30 nucleotides in length. Following post-transcriptional processing, the small hairpin RNA is converted into a siRNA by a cleavage event mediated by the enzyme DICER, which is a member of the RNase III family. The siRNA is then capable of inhibiting the expression of a gene with which it shares homology. Further details are described by see Brummelkamp et al. (2002), Science, 296:550-553; Lee et al. (2002), Nature Biotechnol., 20, 500-505; Miyagishi and Taira (2002), Nature Biotechnol., 20:497-500; Paddison et al. (2002), Genes & Dev., 16:948-958; Paul (2002), Nature Biotechnol., 20, 505-508; Sui (2002), Proceedings Nat'l Acad. Sci. USA, 99(6), 5515-5520; and Yu et al. (2002), Proceedings Nat'l Acad. Sci. USA 99:6047-6052, the disclosures of which are hereby incorporated by reference herein in their entireties.

2.4.2 shRNA

In some embodiments, the second nucleic acid sequence encodes a shRNA that inhibits HPRT. In a particular embodiment, the shRNA is sh734, such as one comprising a sequence set forth in SEQ ID NO:66 or a sequence having at least 85%, 86%, 87%, 88%, 89%, 90%, 91%, 92%, 93%, 94%, 95%, 96%, 97% or 99% sequence identity thereto. In another embodiment, the sh734 comprises a multi-t termination sequence, which may be required for required for Pol III promoters such as 7SK. Thus, in some embodiments, the sh734 comprises the sequence set forth in SEQ ID NO:67 or a sequence having at least 85%, 86%, 87%, 88%, 89%, 90%, 91%, 92%, 93%, 94%, 95%, 96%, 97% or 99% sequence identity thereto. In further embodiment, the sh734 comprises a single-t termination sequence, and thus comprises, for example, a sequence set forth in SEQ ID NO:68 or a sequence having at least 85%, 86%, 87%, 88%, 89%, 90%, 91%, 92%, 93%, 94%, 95%, 96%, 97% or 99% sequence identity thereto.

### 2.4.3 MicroRNAs

MicroRNAs (miRs) are a group of non-coding RNAs which post-transcriptionally regulate the expression of their target genes. It is believed that these single stranded molecules form a miRNA-mediated silencing complex (miRISC) complex with other proteins which bind to the 3′ untranslated region (UTR) of their target mRNAs so as to prevent their translation in the cytoplasm.

In some embodiments, shRNA sequences are embedded into micro-RNA secondary structures (“micro-RNA based shRNA”). In some embodiments, shRNA nucleic acid sequences targeting HPRT are embedded within micro-RNA secondary structures. In some embodiments, the micro-RNA based shRNAs target coding sequences within HPRT to achieve knockdown of HPRT expression, which is believed to be equivalent to the utilization of shRNA targeting HPRT without attendant pathway saturation and cellular toxicity or off-target effects. In some embodiments, the micro-RNA based shRNA is a de novo artificial microRNA shRNA. The production of such de novo micro-RNA based shRNAs are described by Fang, W. & Bartel, David P. The Menu of Features that Define Primary MicroRNAs and Enable De Novo Design of MicroRNA Genes. Molecular Cell 60, 131-145, the disclosure of which is hereby incorporated by reference herein in its entirety.

Exemplary miRNAs are provided in International Patent Publication No. WO2020139796.

### 2.4.4 Alternatives to RNAi

As an alternative to the incorporation of a RNAi, in some embodiments, the vectors may include a nucleic acid sequence which encodes antisense oligonucleotides that bind sites in messenger RNA (mRNA). Antisense oligonucleotides of the present disclosure specifically hybridize with a nucleic acid encoding a protein and interfere with transcription or translation of the protein. In some embodiments, an antisense oligonucleotide targets DNA and interferes with its replication and/or transcription. In other embodiments, an antisense oligonucleotide specifically hybridizes with RNA, including pre-mRNA (i.e. precursor mRNA which is an immature single strand of mRNA), and mRNA. Such antisense oligonucleotides may affect, for example, translocation of the RNA to the site of protein translation, translation of protein from the RNA, splicing of the RNA to yield one or more mRNA species, and catalytic activity that may be engaged in or facilitated by the RNA. The overall effect of such interference is to modulate, decrease, or inhibit target protein expression.

### 2.5 Other Elements

Other elements that can be present in the lentiviral vectors of the present disclosure include, for example, promoters, operators, termination signals, polyadenylation signals, etc. Those skilled in the art can readily identify suitable elements for the correct processing, transcription and/or translation of nucleic acid present in and encoded by the vectors.

In one example, the vector comprises a Woodchuck Hepatitis Virus (WHV) Posttranscriptional Regulatory Element (WPRE). In a particular embodiment, the WPRE is downstream of the first nucleic acid sequence and upstream of the modified HS4-650 insulator (i.e. is between the first nucleic acid sequence and the modified HS4-650 insulator. In some embodiments, the WPRE is a WPRE mut6 comprising a sequence set forth in SEQ ID NO:77 or a WPRE mut7 comprising a sequence set forth in SEQ ID NO:78, or comprises a sequence having at least 85%, 86%, 87%, 88%, 89%, 90%, 91%, 92%, 93%, 94%, 95%, 96%, 97% or 99% sequence identity to the sequence set forth in SEQ ID NO:77 or 78.

In some examples, the promoter is a MND promoter, such as one comprising a sequence set forth in SEQ ID NO:72 a sequence having at least 85%, 86%, 87%, 88%, 89%, 90%, 91%, 92%, 93%, 94%, 95%, 96%, 97% or 99% sequence identity to the sequence set forth in SEQ ID NO:77 or 78. In one embodiment, the first promoter is a MND promoter and is operably linked to the first nucleic acid comprising the transgene.

In some examples, the promoter is a 7SK RNA promoter, such as one set forth in any one of SEQ ID NOs:69-71, or one comprising a sequence having at least 85%, 86%, 87%, 88%, 89%, 90%, 91%, 92%, 93%, 94%, 95%, 96%, 97% or 99% sequence identity to the sequences set forth in SEQ ID NO:69-71. In one embodiment, the second promoter is a 7SK RNA promoter and is operably linked to the second nucleic acid encoding a nucleic acid that inhibits HPRT expression.

In some examples, the lentiviral vector comprises a 7tetO promoter/operator, such as one comprising a sequence set forth in SEQ ID NO:79 or a sequence having at least 85%, 86%, 87%, 88%, 89%, 90%, 91%, 92%, 93%, 94%, 95%, 96%, 97% or 99% sequence identity to the sequence set forth in SEQ ID NO:79.

In further examples, the lentiviral vector comprises a β-globin poly(A) signal, such as one comprising a sequence set forth in SEQ ID NO:80 or a sequence having at least 85%, 86%, 87%, 88%, 89%, 90%, 91%, 92%, 93%, 94%, 95%, 96%, 97% or 99% sequence identity to the sequence set forth in SEQ ID NO:80.

### 2.6 Production of Vectors

The lentiviral vectors of the present disclosure can be produced using any method, and such methods are well known to those skilled in the art. In some embodiments, the first promoter operably linked to the first nucleic acid sequence encoding a therapeutic protein such a Wiskott-Aldrich Syndrome protein; and a modified HS4-650 insulator (and optionally any other expression cassette or element described herein) is inserted into a lentiviral vector that is a plasmid, such as one selected from the group consisting of pTL20c, pTL20d, FG, pRRL, pCL20, pLKO.1 puro, pLKO.1, pLKO.3G, Tet-pLKO-puro, pSico, pLJM1-EGFP, FUGW, pLVTHM, pLVUT-tTR-KRAB, pLL3.7, pLB, pWPXL, pWPI, EF.CMV.RFP, pLenti CMV Puro DEST, pLenti-puro, pLOVE, pULTRA, pLJM1-EGFP, pLX301, pInducer20, pHIV-EGFP, Tet-pLKO-neo, pLV-mCherry, pCW57.1, pLionII, pSLIK-Hygro, and pInducer10-mir-RUP-PheS. In other embodiments, the lentiviral vector into which the first promoter, the first nucleic acid sequence and the modified HS4-650 insulator is inserted is selected from AnkT9W vector, a T9Ank2W vector, a TNS9 vector, a lentiglobin HPV569 vector, a lentiglobin BB305 vector, a BG-1 vector, a BGM-1 vector, a GLOBE vector, a G-GLOBE vector, a V5 vector, a V5m3 vector, a V5m3-400 vector, a G9 vector, and a BCL11A shmir vector. In a particular embodiment, the lentiviral expression vector is pTL20c.

In one example, an expression cassette having the first promoter operably linked to the first nucleic acid sequence, and a modified HS4-650 insulator, may be inserted into a pTL20c vector according to the methods described in United States Patent Publication No. 20180112233 and International Patent Publication No. WO2020139796.

In some examples, an expression cassette having the first promoter operably linked to the first nucleic acid sequence, and optionally a modified HS4-400 insulator, may be inserted into a pTL20c vector according to the methods described in United States Patent Publication No. 20180112233 and International Patent Publication No. WO2020139796.

Lentivirus particles or virions (or recombinant lentiviruses) can be produced using standard methods well known in the art. In one example, a stable producer cell line for generating virus is utilized, wherein the stable producer cell line is derived from one of a GPR, GPRG, GPRT, GPRGT, or GPRT-G packing cell line. In some embodiments, the stable producer cell line is derived from the GPRT-G cell line. In some embodiments, the stable producer cell line is generated by (a) synthesizing a vector by cloning nucleic acid sequences encoding an anti-HPRT shRNA and WASP into a recombinant plasmid (i.e. the synthesized vector may be any one of the vectors described herein that encode an anti-HPRT shRNA and WASP); (b) generating DNA fragments from the synthesized vector; (c) forming a concatemeric array from (i) the generated DNA fragments from the synthesized vector, and (ii) from DNA fragments derived from an antibiotic resistance cassette plasmid; (d) transfecting one of the packaging cell lines with the formed concatemeric array; and (e) isolating the stable producer cell line. Additional methods of forming a stable producer cell line are described in United States Patent Publication No. 20180112233.

### 2.7 Exemplary Vectors

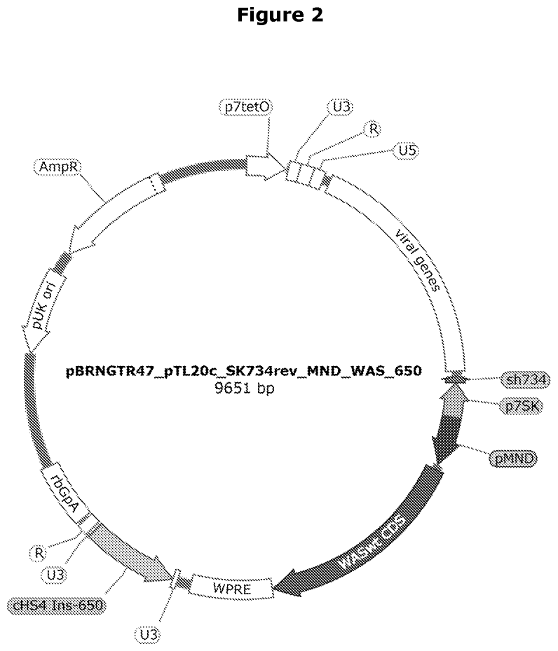

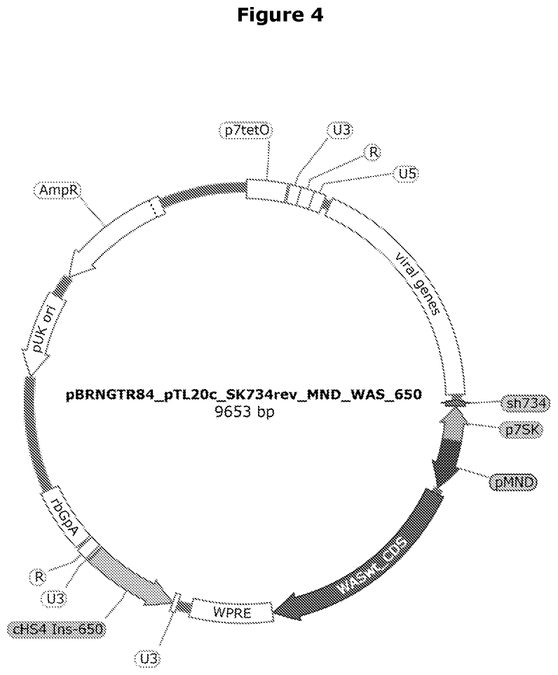

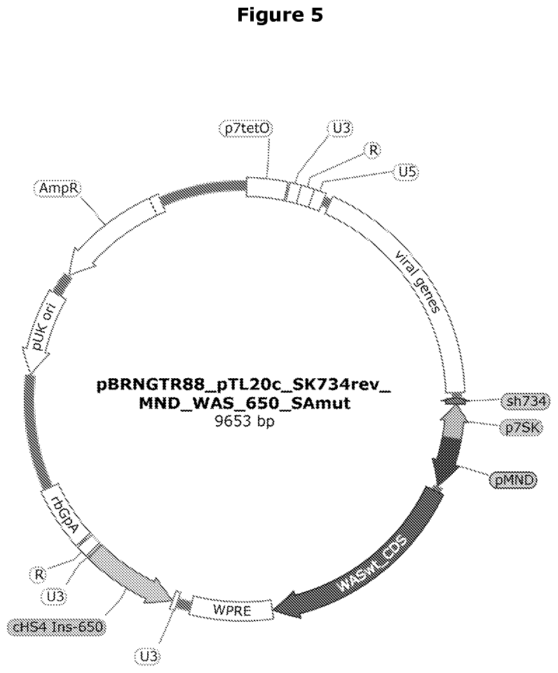

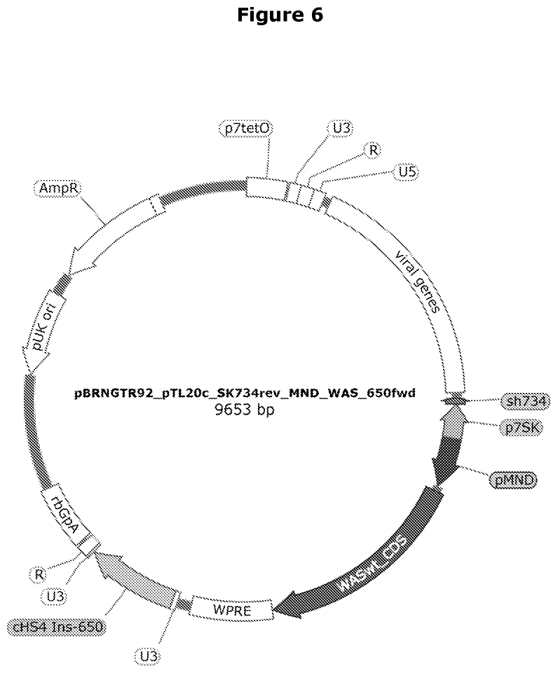

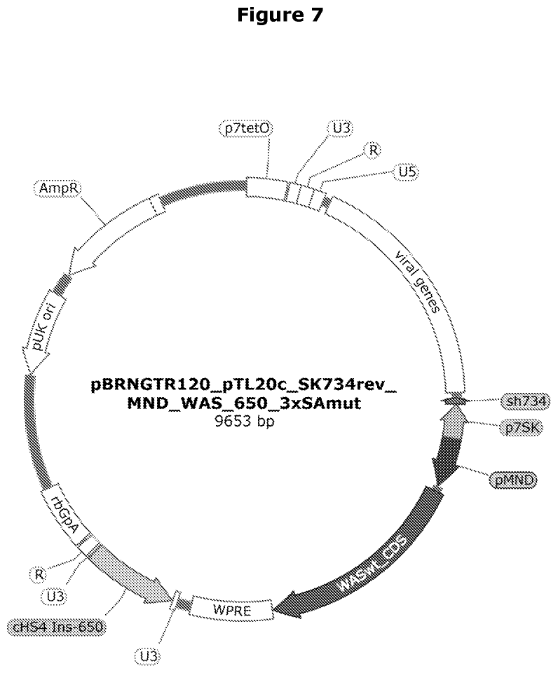

Exemplary lentiviral vectors of the present disclosure include nucleic acid vectors (e.g. plasmids) and lentivirus virions (or virus particles) that comprise a 5′LTR (including a 7tetO promoter/operator, R and U5, such as shown schematically in FIGS. 2-7) downstream of which, from 5′ to 3′, is a central polypurine tract (cPPT), a REV response element (RRE), a 7sk-sh734 expression cassette comprising a 7sk promoter (e.g. one comprising a sequence set forth in any one of SEQ ID NOs:69-72 or a sequence having at least 85%, 86%, 87%, 88%, 89%, 90%, 91%, 92%, 93%, 94%, 95%, 96%, 97% or 99% sequence identity thereto) operably linked to nucleic acid encoding sh734 (e.g. one encoding a sh734 comprising a sequence set forth in any one of SEQ ID NOs:66-68 or a sequence having at least 85%, 86%, 87%, 88%, 89%, 90%, 91%, 92%, 93%, 94%, 95%, 96%, 97% or 99% sequence identity thereto), a WASP expression cassette comprising a MND promoter (e.g. one comprising the sequence set forth in SEQ ID NO: 72 or a sequence having at least 85%, 86%, 87%, 88%, 89%, 90%, 91%, 92%, 93%, 94%, 95%, 96%, 97% or 99% sequence identity thereto) operably linked to a transgene encoding WASP (such as a transgene comprising the sequence set forth in any one of SEQ ID NOs: 73-75 or a sequence having at least 85%, 86%, 87%, 88%, 89%, 90%, 91%, 92%, 93%, 94%, 95%, 96%, 97% or 99% sequence identity thereto), a WPRE (e.g. one comprising the sequence set forth in SEQ ID NO: 77 or 78 or a sequence having at least 85%, 86%, 87%, 88%, 89%, 90%, 91%, 92%, 93%, 94%, 95%, 96%, 97% or 99% sequence identity thereto), and a 3′LTR, which includes a HS4-650 insulator (such as an unmodified HS4-650 insulator set forth in any one of SEQ ID NOs:1, 10, 19, 28, 37 and 46 or a sequence having at least 85%, 86%, 87%, 88%, 89%, 90%, 91%, 92%, 93%, 94%, 95%, 96%, 97% or 99% sequence identity thereto, wherein the HS4-650 insulator is in the forward orientation, or a modified HS4-650 insulator described herein having an inactivated SA1, SA2 and/or SA3, wherein the modified HS4-650 insulator is in the reverse orientation, e.g. one comprising a complementary strand comprising the sequence set forth in any one of SEQ ID NOs: 3, 12, 21, 30, 39 and 48 or a sequence having at least 85%, 86%, 87%, 88%, 89%, 90%, 91%, 92%, 93%, 94%, 95%, 96%, 97% or 99% sequence identity thereto, wherein the sequence comprises a T at position 384 with numbering relative to SEQ ID NO:2; one comprising a complementary strand comprising the sequence set forth in any one of SEQ ID NOs: 7, 16, 25, 34, 43 and 52 or a sequence having at least 85%, 86%, 87%, 88%, 89%, 90%, 91%, 92%, 93%, 94%, 95%, 96%, 97% or 99% sequence identity thereto, wherein the sequence comprises a T at position 445 with numbering relative to SEQ ID NO:2; one comprising a complementary strand comprising the sequence set forth in any one of SEQ ID NOs: 9, 18, 27, 36, 45 and 54 or a sequence having at least 85%, 86%, 87%, 88%, 89%, 90%, 91%, 92%, 93%, 94%, 95%, 96%, 97% or 99% sequence identity thereto, wherein the sequence comprises a T at position 455 with numbering relative to SEQ ID NO:2; one comprising a complementary strand comprising the sequence set forth in any one of SEQ ID NOs: 4, 13, 22, 31, 40 and 49 or a sequence having at least 85%, 86%, 87%, 88%, 89%, 90%, 91%, 92%, 93%, 94%, 95%, 96%, 97% or 99% sequence identity thereto, wherein the sequence comprises a T at position 384 and a T at position 445, with numbering relative to SEQ ID NO:2; one comprising a complementary strand comprising the sequence set forth in any one of SEQ ID NOs: 5, 14, 23, 32, 41 and 50 or a sequence having at least 85%, 86%, 87%, 88%, 89%, 90%, 91%, 92%, 93%, 94%, 95%, 96%, 97% or 99% sequence identity thereto, wherein the sequence comprises T at position 384 and a T at position 455, with numbering relative to SEQ ID NO:2; one comprising a complementary strand comprising the sequence set forth in any one of SEQ ID NOs: 8, 17, 26, 35, 44 and 43 or a sequence having at least 85%, 86%, 87%, 88%, 89%, 90%, 91%, 92%, 93%, 94%, 95%, 96%, 97% or 99% sequence identity thereto, wherein the sequence comprises T at position 445 and a T at position 455, with numbering relative to SEQ ID NO:2; or one comprising a complementary strand comprising the sequence set forth in any one of SEQ ID NOs: 6, 15, 24, 33, 42 and 51 or a sequence having at least 85%, 86%, 87%, 88%, 89%, 90%, 91%, 92%, 93%, 94%, 95%, 96%, 97% or 99% sequence identity thereto, wherein the sequence comprises a T at position 384, a T at position 445 and a T at position 455, with numbering relative to SEQ ID NO:2), U3, R and a β-globin poly(A) signal (e.g. one comprising the sequence set forth in SEQ ID NO: 31 or a sequence having at least 85%, 86%, 87%, 88%, 89%, 90%, 91%, 92%, 93%, 94%, 95%, 96%, 97% or 99% sequence identity thereto). In these vectors, typically the 7sk-sh734 expression cassette is in the reverse orientation and the WASP expression cassette is in the forward orientation.

In some embodiments, the lentiviral vectors comprise a sequence selected from the group consisting of: the sequence set forth as nucleotides 3098-6006 of SEQ ID NO:57 or a sequence having at least 85%, 86%, 87%, 88%, 89%, 90%, 91%, 92%, 93%, 94%, 95%, 96%, 97%, 98% or 99% sequence identity thereto, wherein the sequence comprises an inactivation of SA1 and SA2, e.g. comprises a T at position 384 and a T at position 445, with numbering relative to SEQ ID NO:2; the sequence set forth as nucleotides 3098-6009 of SEQ ID NO:58 or a sequence having at least 85%, 86%, 87%, 88%, 89%, 90%, 91%, 92%, 93%, 94%, 95%, 96%, 97%, 98% or 99% sequence identity thereto; and the sequence set forth as nucleotides 3098-6006 of SEQ ID NO:59 or a sequence having at least 85%, 86%, 87%, 88%, 89%, 90%, 91%, 92%, 93%, 94%, 95%, 96%, 97%, 98% or 99% sequence identity thereto, wherein the polynucleotide comprises an inactivation of SA1, SA2 and SA3, wherein the sequence comprises an inactivation of SA1, SA2 and SA3, e.g. comprises a T at position 384, a T at position 445 and a T at position 455, with numbering relative to SEQ ID NO:2.

In some embodiments, the lentiviral vectors comprise a sequence selected from the group consisting of: the sequence set forth as nucleotides 2710-6006 of SEQ ID NO:57 or a sequence having at least 85%, 86%, 87%, 88%, 89%, 90%, 91%, 92%, 93%, 94%, 95%, 96%, 97%, 98% or 99% sequence identity thereto, wherein the sequence comprises an inactivation of SA1 and SA2, e.g. comprises a T at position 384 and a T at position 445, with numbering relative to SEQ ID NO:2; the sequence set forth as nucleotides 2710-6009 of SEQ ID NO:58 or a sequence having at least 85%, 86%, 87%, 88%, 89%, 90%, 91%, 92%, 93%, 94%, 95%, 96%, 97%, 98% or 99% sequence identity thereto; and the sequence set forth as nucleotides 2710-6006 of SEQ ID NO:59 or a sequence having at least 85%, 86%, 87%, 88%, 89%, 90%, 91%, 92%, 93%, 94%, 95%, 96%, 97%, 98% or 99% sequence identity thereto, wherein the sequence comprises an inactivation of SA1, SA2 and SA3, e.g. comprises a T at position 384, a T at position 445 and a T at position 455, with numbering relative to SEQ ID NO:2.

In some embodiments, the lentiviral vectors comprise a sequence selected from the group consisting of: the sequence set forth as nucleotides 2402-6006 of SEQ ID NO:57 or a sequence having at least 85%, 86%, 87%, 88%, 89%, 90%, 91%, 92%, 93%, 94%, 95%, 96%, 97%, 98% or 99% sequence identity thereto, wherein the sequence comprises an inactivation of SA1 and SA2, e.g. comprises a T at position 384 and a T at position 445, with numbering relative to SEQ ID NO:2; the sequence set forth as nucleotides 2402-6009 of SEQ ID NO:58 or a sequence having at least 85%, 86%, 87%, 88%, 89%, 90%, 91%, 92%, 93%, 94%, 95%, 96%, 97%, 98% or 99% sequence identity thereto; and the sequence set forth as nucleotides 2402-6006 of SEQ ID NO:59 or a sequence having at least 85%, 86%, 87%, 88%, 89%, 90%, 91%, 92%, 93%, 94%, 95%, 96%, 97%, 98% or 99% sequence identity thereto, wherein the polynucleotide comprises an inactivation of SA1, SA2 and SA3, wherein the sequence comprises an inactivation of SA1, SA2 and SA3, e.g. comprises a T at position 384, a T at position 445 and a T at position 455, with numbering relative to SEQ ID NO:2.

In one embodiment, the lentiviral vectors of the present disclosure comprise a sequence set forth in SEQ ID NO:57 or 82 or a sequence having at least 85%, 86%, 87%, 88%, 89%, 90%, 91%, 92%, 93%, 94%, 95%, 96%, 97%, 98% or 99% sequence identity thereto, wherein the sequence comprises an inactivation of SA1 and SA2, e.g. comprises a T at position 384 and a T at position 445, with numbering relative to SEQ ID NO:2.

In another embodiment, the lentiviral vectors of the present disclosure comprise a sequence set forth in SEQ ID NO:58 or 83 or a sequence having at least 85%, 86%, 87%, 88%, 89%, 90%, 91%, 92%, 93%, 94%, 95%, 96%, 97%, 98% or 99% sequence identity thereto.

In another embodiment, the lentiviral vectors of the present disclosure comprise a sequence set forth in SEQ ID NO:59 or 84 or a sequence having at least 85%, 86%, 87%, 88%, 89%, 90%, 91%, 92%, 93%, 94%, 95%, 96%, 97%, 98% or 99% sequence identity thereto, wherein the sequence comprises an inactivation of SA1, SA2 and SA3, e.g. comprises a T at position 384, a T at position 445 and a T at position 455, with numbering relative to SEQ ID NO:2.

### 3. Host Cells

The present disclosure also provides a host cell comprising, transformed or transduced with a lentiviral vector of the present disclosure. A “host cell” or “target cell” means a cell that is to be transformed or transduced using the methods and vectors of the present disclosure. In some embodiments, the host cells are mammalian cells in which the vector can be expressed. Suitable mammalian host cells include, but are not limited to, human cells, murine cells, non-human primate cells (e.g. rhesus monkey cells), human progenitor cells or stem cells, 293 cells, HeLa cells, D17 cells, MDCK cells, BHK cells, and Cf2Th cells. In certain embodiments, the host cell comprising an expression vector of the disclosure is a hematopoietic cell, such as hematopoietic progenitor/stem cell (e.g. CD34-positive hematopoietic progenitor/stem cell), a monocyte, a macrophage, a peripheral blood mononuclear cell, a CD4+T lymphocyte, a CD8+T lymphocyte, or a dendritic cell.

The hematopoietic stem cells (e.g. CD4+T lymphocytes, CD8+T lymphocytes, and/or monocyte/macrophages) to be transduced with a vector of the disclosure can be allogeneic, autologous, or from a matched sibling. The HSCs are, in some embodiments, CD34-positive and can be isolated from the patient's bone marrow or peripheral blood. The isolated CD34-positive HSCs (and/or other hematopoietic cell described herein) is, in some embodiments, transduced with an vector as described herein.

In some embodiments, the host cells or transduced host cells are combined with a pharmaceutically acceptable carrier. In some embodiments, the host cells or transduced host cells are formulated with PLASMA-LYTE A (e.g. a sterile, nonpyrogenic isotonic solution for intravenous administration; where one liter of PLASMA-LYTE A has an ionic concentration of 140 mEq sodium, 5 mEq potassium, 3 mEq magnesium, 98 mEq chloride, 27 mEq acetate, and 23 mEq gluconate). In other embodiments, the host cells or transduced host cells are formulated in a solution of PLASMA-LYTE A, the solution comprising between about 8% and about 10% dimethyl sulfoxide (DMSO). In some embodiments, the less than about 2×10⁷ host cells/transduced host cells are present per mL of a formulation including PLASMA-LYTE A and DMSO.

In some embodiments, the host cells are rendered substantially HPRT deficient after transduction with a vector according to the present disclosure. In some embodiments, the level of HPRT gene expression is reduced by at least 50%. In some embodiments, the level of HPRT gene expression is reduced by at least 55%. In some embodiments, the level of HPRT gene expression is reduced by at least 60%. In some embodiments, the level of HPRT gene expression is reduced by at least 65%. In some embodiments, the level of HPRT gene expression is reduced by at least 70%. In some embodiments, the level of HPRT gene expression is reduced by at least 75%. In some embodiments, the level of HPRT gene expression is reduced by at least 80%. In some embodiments, the level of HPRT gene expression is reduced by at least 85%. In some embodiments, the level of HPRT gene expression is reduced by at least 90%. In some embodiments, the level of HPRT gene expression is reduced by at least 95%. It is believed that cells having 20% or less residual HPRT gene expression are sensitive to a purine analog, such as 6TG, allowing for their selection with the purine analog.

In some embodiments, transduction of host cells may be increased by contacting the host cell, in vitro, ex vivo, or in vivo, with an expression vector of the present disclosure and one or more compounds that increase transduction efficiency. For example, in some embodiments, the one or more compounds that increase transduction efficiency are compounds that stimulate the prostaglandin EP receptor signaling pathway, i.e. one or more compounds that increase the cell signaling activity downstream of a prostaglandin EP receptor in the cell contacted with the one or more compounds compared to the cell signaling activity downstream of the prostaglandin EP receptor in the absence of the one or more compounds. In some embodiments, the one or more compounds that increase transduction efficiency are a prostaglandin EP receptor ligand including, but not limited to, prostaglandin E2 (PGE2), or an analog or derivative thereof. In other embodiments, the one or more compounds that increase transduction efficiency include but are not limited to, RetroNectin (a 63 kD fragment of recombinant human fibronectin fragment, available from Takara); Lentiboost (a membrane-sealing poloxamer, available from Sirion Biotech), Protamine Sulphate, Cyclosporin H, and Rapamycin.

### 4. Pharmaceutical Compositions

The present disclosure also provides for compositions, including pharmaceutical compositions, comprising one or more vectors and/or non-viral delivery vehicles (e.g. nanocapsules) as disclosed herein. In some embodiments, pharmaceutical compositions comprise an effective amount of at least one of the vectors and/or non-viral delivery vehicles as described herein and a pharmaceutically acceptable carrier. For instance, in certain embodiments, the pharmaceutical composition comprises an effective amount of an vector and a pharmaceutically acceptable carrier. An effective amount can be readily determined by those skilled in the art based on factors such as body size, body weight, age, health, sex of the subject, ethnicity, and viral titers.

The phrases “pharmaceutically acceptable” or “pharmacologically acceptable” refer to molecular entities and compositions that do not produce adverse, allergic, or other untoward reactions when administered to an animal or a human. For example, an expression vector may be formulated with a pharmaceutically acceptable carrier. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. Methods for the formulation of compounds with pharmaceutical carriers are known in the art and are described in, for example, in Remington's Pharmaceutical Science, (17th ed. Mack Publishing Company, Easton, Pa. 1985); and Goodman & Gillman's: The Pharmacological Basis of Therapeutics (11th Edition, McGraw-Hill Professional, 2005); the disclosures of each of which are hereby incorporated herein by reference in their entirety.

In some embodiments, the pharmaceutical compositions may comprise any of the vectors, nanocapsules, or compositions disclosed herein in any concentration that allows the silencing nucleic acid administered to achieve a concentration in the range of from about 0.1 mg/kg to about 1 mg/kg. In some embodiments, the pharmaceutical compositions may comprise the expression vector in an amount of from about 0.1% to about 99.9% by weight. Pharmaceutically acceptable carriers suitable for inclusion within any pharmaceutical composition include water, buffered water, saline solutions such as, for example, normal saline or balanced saline solutions such as Hank's or Earle's balanced solutions), glycine, hyaluronic acid etc. The pharmaceutical composition may be formulated for parenteral administration, such as intravenous, intramuscular or subcutaneous administration. Pharmaceutical compositions for parenteral administration may comprise pharmaceutically acceptable sterile aqueous or non-aqueous solutions, dispersions, suspensions or emulsions as well as sterile powders for reconstitution into sterile injectable solutions or dispersions. Examples of suitable aqueous and non-aqueous carriers, solvents, diluents or vehicles include water, ethanol, polyols (such as glycerol, propylene glycol, polyethylene glycol, etc.), carboxymethylcellulose and mixtures thereof, vegetable oils (such as olive oil), injectable organic esters (e.g. ethyl oleate).

The pharmaceutical composition may be formulated for oral administration. Solid dosage forms for oral administration may include, for example, tablets, dragees, capsules, pills, and granules. In such solid dosage forms, the composition may comprise at least one pharmaceutically acceptable carrier such as sodium citrate and/or dicalcium phosphate and/or fillers or extenders such as starches, lactose, sucrose, glucose, mannitol, and silicic acid; binders such as carboxylmethylcellulose, alginates, gelatin, polyvinylpyrrolidone, sucrose and acacia; humectants such as glycerol; disintegrating agents such as agar-agar, calcium carbonate, potato or tapioca starch, alginic acid, silicates, and sodium carbonate; wetting agents such as acetyl alcohol, glycerol monostearate; absorbants such as kaolin and bentonite clay; and/or lubricants such as talc, calcium stearate, magnesium stearate, solid polyethylene glycol, sodium lauryl sulfate, and mixtures thereof. Liquid dosage forms for oral administration may include, for example, pharmaceutically acceptable emulsions, solutions, suspensions, syrups and elixirs. Liquid dosages may include inert diluents such as water or other solvents, solubilizing agents and/or emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (such as, for example, cottonseed oil, corn oil, germ oil, castor oil, olive oil, sesame oil), glycerol, tetrahydrofurfuryl alcohol, polyethylene glycols and fatty acid esters of sorbitan, and mixtures thereof.

The pharmaceutical compositions may comprise penetration enhancers to enhance their delivery. Penetration enhancers may include fatty acids such as oleic acid, lauric acid, capric acid, myristic acid, palmitic acid, stearic acid, linoleic acid, linolenic acid, dicaprate, reclineate, monoolein, dilaurin, caprylic acid, arachidonic acid, glyceryl 1-monocaprate, mono and di-glycerides and physiologically acceptable salts thereof. The compositions may further include chelating agents such as, for example, ethylenediaminetetraacetic acid (EDTA), citric acid, salicylates (e.g. sodium salicylate, 5-methoxysalicylate, homovanilate).

The pharmaceutical compositions may comprise any of the vectors disclosed herein in an encapsulated form. For example, the vectors may be encapsulated within a nanocapsule, such as a nanocapsule comprising one or more biodegradable polymers such as polylactide-polyglycolide, poly(orthoesters) and poly(anhydrides). In some embodiments, the vectors are encapsulated within polymeric nanocapsules. In other embodiments, the vectors are encapsulated within biodegradable and/or erodible polymeric nanocapsules. In some embodiments, the polymeric nanocapsules are comprised of two different positively charged monomers, at least one neutral monomer, and a crosslinker. In some embodiments, the nanocapsules further comprise at least one targeting moiety. In some embodiments, the nanocapsules comprise between 2 and between 6 targeting moieties. In some embodiments, the taretinc moieties are antibodies. In some embodiments, the targeting moieties target any one of the CD117, CD10, CD34, CD38, CD45, CD123, CD127, CD135, CD44, CD47, CD96, CD2, CD4, CD3, and CD9 markers. In some embodiments, the targeting moiety targets any one of a human mesenchymal stem cell CD marker, including the CD29, CD44, CD90, CD49a-f, CD51, CD73 (SH3), CD105 (SH2), CD106, CD166, and Stro-1 markers. In some embodiments, the targeting moiety targets any one of a human hematopoietic stem cell CD marker including CD34, CD38, CD45RA, CD90, and CD49.

### 5. Methods of Treatment

By way of example, a lentiviral vector described herein comprising a nucleic acid sequence encoding WASP may be administered so as to genetically correct Wiskott-Aldrich Syndrome or to alleviate the pathologies associated with Wiskott-Aldrich Syndrome. In some embodiments, a population of host cells transduced with a vector is administered so as to correct Wiskott-Aldrich Syndrome or to alleviate the pathologies associated with Wiskott-Aldrich Syndrome. It is believed that this method is advantageous over currently available therapies, due to its availability to all patients, particularly those who do not have a matched sibling donor. It is further believed that this method also has the potential to be administered as a one-time treatment providing lifelong correction. It is also believed that the method is advantageously devoid of any immune side effects, and if side effects did arise, the side-effects could be mitigated by administering a dihydrofolate reductase inhibitor (e.g. MTX or MPA) as noted herein. It is further believed that an effective gene therapy approach will revolutionize the way Wiskott-Aldrich Syndrome is treated, ultimately improving patient outcome.

In some embodiments, treatment with the vectors or transduced host cells described herein genetically corrects or alleviates one or more of the pathologies associated with Wiskott-Aldrich Syndrome, such as those outlined below. In some embodiments, the pathologies which may be genetically corrected or alleviated by administering the expression vectors or transduced host cells to a patient include, but are not limited to, microthrombocytopenia, eczema, autoimmune diseases, and recurrent infections. An eczema rash is common in patients with classic WAS. In infants, the eczema may occur on the face or scalp and can resemble “cradle cap.” It can also have the appearance of a severe diaper rash, or be more generalized, involving the arms and legs. In older boys, eczema is often limited to the skin creases around the front of the elbows or behind the knees, behind the ears, or around the wrist. Since eczema is extremely itchy, patients often scratch themselves until they bleed, even while asleep. These areas where the skin barrier is broken can then serve as entry points for bacteria that can cause skin and blood stream infections.

It is believed that thrombocytopenia (a reduced number of platelets) is a common feature of patients with Wiskott-Aldrich Syndrome. In addition to being decreased in number, the platelets themselves are small and dysfunctional, less than half the size of normal platelets. As a result, patients with Wiskott-Aldrich Syndrome may bleed easily, even if they have not had an injury. In some embodiments, bleeding into the skin may cause pinhead sized bluish-red spots, called petechiae, or they may be larger and resemble bruises.

It is believed that the immunodeficiency associated with Wiskott-Aldrich Syndrome causes the function of both B- and T-lymphocytes to be significantly abnormal. As a result, infections are common in the classic form of Wiskott-Aldrich Syndrome and may involve all classes of microorganisms. In some embodiments, these infections may include upper and lower respiratory infections such as ear infections, sinus infections and pneumonia. More severe infections such as sepsis (bloodstream infection or “blood poisoning”), meningitis and severe viral infections are less frequent but can occur. Occasionally, patients with the classic form of Wiskott-Aldrich Syndrome may develop pneumonia caused by the fungus (Pneumocystis jiroveci carinii). In some embodiments, the skin may become infected with bacteria such as Staphylococcus in areas where patients have scratched their eczema. In some embodiments, a viral skin infection called molluscum contagiosum is also commonly seen in Wiskott-Aldrich Syndrome. It is believed that vaccination to prevent infections is often not effective in Wiskott-Aldrich Syndrome since patients do not make normal protective antibody responses to vaccines.

In some embodiments, the recurrent infections include, but are not limited to, otitis media, skin abscess, pneumonia, enterocolitis, meningitis, sepsis, and urinary tract infection. In some embodiments, the recurrent infections are cutaneous infections. In some embodiments, the eczema experienced by patients diagnosed with Wiskott-Aldrich Syndrome is classified as treatment-resistant eczema.

By way of example, autoimmune diseases often experienced by those having Wiskott-Aldrich Syndrome include hemolytic anemia, vasculitis, arthritis, neutropenia, inflammatory bowel disease, and IgA nephropathy, Henoch-Schönlein-like purpura, dermatomyositis, recurrent angioedema, and uveitis. In some embodiments, the recurrent infections may be caused by any of a bacterial, viral, or fungal infection. In some embodiments, treatment with the vectors or transduced host cells described herein genetically corrects or alleviates a plurality of the pathologies associated with Wiskott-Aldrich Syndrome, such as those outlined below.

As noted herein, in addition to the therapeutic gene, the expression vectors of the present disclosure include an agent designed to inhibit or knockdown HPRT expression (e.g. a shRNA to HPRT), and hence provide for an in vivo chemoselection strategy that exploits the essential role that HPRT plays in metabolizing purine analogs, e.g. 6TG, into myelotoxic agents. Because HPRT-deficiency does not impair hematopoietic cell development or function, it can be removed from hematopoietic cells used for transplantation. Conditioning and chemoselection with a purine analog are discussed further herein.

In the context of the treatment of or alleviation of the pathologies associated with Wiskott-Aldrich Syndrome, the treatment of a subject includes: identifying a subject in need of treatment thereof; transducing HSCs (e.g. autologous HSCs, allogenic HSCs, sibling matched HSCs) with a lentiviral vector of the present disclosure; and transplanting or administering the transduced HSCs into the subject. In some embodiments, the subject in need of treatment thereof is one suffering from the pathologies associated with Wiskott-Aldrich Syndrome.

In some embodiments, the method further comprises a step of myeloablative conditioning prior to the administration of the transduced HSCs (e.g. using a purine analog, chemotherapy, radiation therapy, treatment with one or more internalizing immunotoxins or antibody-drug conjugates, or any combination thereof). In some embodiments, the method further comprises the step of pre-conditioning, or in vivo chemoselection, utilizing a purine analog (e.g. 6TG) following administration of the transduced HSCs. In some embodiments, the method further comprises the step of negative selection utilizing a dihydrofolate reductase inhibitor (e.g. MTX or MPA) should side effects arise (e.g. GvHD).

5.1 Conditioning and Chemoselection with a Purine Analog

In some embodiments, the method of treatment comprises the additional steps of (i) conditioning prior to HSC transplantation; and/or (ii) in vivo chemoselection. One or both steps may utilize a purine analog. In some embodiments, the purine analog is selected from the group consisting of 6-thioguanine (“6TG”), 6-mercaptopurine (“6MP”) or azathiopurine (“AZA”). It is believed that the engrafted Wiskott-Aldrich Syndrome protein-containing HSCs deficient in HPRT activity are highly resistant to the cytotoxic effects of the introduced purine analog. With a combined strategy of conditioning and chemoselection, efficient and high engraftment of HPRT-deficient, Wiskott-Aldrich Syndrome protein-containing HSCs with low overall toxicity can be achieved. It is believed that resultant expression of the Wiskott-Aldrich Syndrome protein, combined with the enhanced engraftment and chemoselection of gene-modified HSCs, can result in sufficient protein production to alleviate the pathologies associated with Wiskott-Aldrich Syndrome.

6TG is a purine analog having both anticancer and immune-suppressive activities. Thioguanine competes with hypoxanthine and guanine for the enzyme hypoxanthine-guanine phosphoribosyltransferase (HGPRTase) and is itself converted to 6-thioguanylic acid (TGMP). This nucleotide reaches high intracellular concentrations at therapeutic doses. TGMP interferes several points with the synthesis of guanine nucleotides. It inhibits de novo purine biosynthesis by pseudo-feedback inhibition of glutamine-5-phosphoribosylpyrophosphateamidotransferase-the first enzyme unique to the de novo pathway for purine ribonucleotide. TGMP also inhibits the conversion of inosinic acid (IMP) to xanthylic acid (XMP) by competition for the enzyme IMP dehydrogenase. At one-time TGMP was felt to be a significant inhibitor of ATP:GMP phosphotransferase (guanylate kinase), but recent results have shown this not to be so. Thioguanylic acid is further converted to the di- and tri-phosphates, thioguanosine diphosphate (TGDP) and thioguanosine triphosphate (TGTP) (as well as their deoxyribosyl analogues) by the same enzymes which metabolize guanine nucleotides.

As those of skill in the art will appreciate, given the inclusion of an agent designed to inhibit HPRT expression, e.g. an RNAi agent to knockdown HPRT, in the vectors of the present disclosure, the resulting transduced HSCs are HPRT-deficient or substantially HPRT-deficient (e.g. such as those having 20% or less residual HPRT gene expression). As such, those HSCs that do express HPRT, i.e. HPRT wild-type cells, may be selectively depleted by administering one or more doses of 6TG. In some embodiments, 6TG may be administered for both myeloablative conditioning of HPRT-wild type recipients and for in vivo chemoselection process of donor cells. Hence, this strategy is believed to allow for the selection of gene-modified cells in vivo, i.e. for the selection of the Wiskott-Aldrich Syndrome protein-containing gene-modified cells in vivo.

In some embodiments, following the collection of HSCs from a donor, the HSCs are transduced with a vector according to the present disclosure. The resulting HSCs are HPRT-deficient and express the WAS gene. In parallel, a patient to receive the HSCs is first treated with a myeloablative conditioning step. Following conditioning, the transduced HSCs are transplanted or administered to the patient. The WAS gene containing HSCs may then be selected for in vivo using 6TG, as discussed herein.

Myeloablative conditioning may be achieved using high-dose conditioning radiation, chemotherapy, and/or treatment with a purine analog (e.g. 6TG). In some embodiments, the HSCs are administered between about 24 and about 96 hours following treatment with the conditioning regimen. In other embodiments, the patient is treated with the HSC graft between about 24 and about 72 hours following treatment with the conditioning regimen. In yet other embodiments, the patient is treated with the HSC graft between about 24 and about 48 hours following treatment with the conditioning regimen. In some embodiments, the HSC graft comprises between about 2×106 cells/kg to about 15×106 cells/kg (body weight of patient). In some embodiments, the HSC graft comprises a minimum of 2×106 cells/kg, with a target of greater than 6×106 cells/kg. In some embodiments, at least 10% of the cells administered are transduced with a lentiviral vector as described herein. In some embodiments, at least 20% of the cells administered are transduced with a lentiviral vector as described herein. In some embodiments, at least 30% of the cells administered are transduced with a lentiviral vector as described herein. In some embodiments, at least 40% of the cells administered are transduced with a lentiviral vector as described herein. In some embodiments, at least 50% of the cells administered are transduced with a lentiviral vector as described herein.

In some embodiments, transgene-containing HPRT-deficient HSCs are selected for in vivo using a low dose schedule of a purine analog, such as 6TG, which is believed to have minimal adverse effects on extra-hematopoietic tissues. In some embodiments, a dosage of the purine analog, such as 6TG, for in vivo chemoselection ranging from between about 0.2 mg/kg/day to about 0.6 mg/kg/day is provided to a patient following introduction of the HSCs into the patient. In some embodiments, the dosage ranges from between about 0.3 mg/kg/day to about 1 mg/kg/day. In some embodiments, the dosage is up to about 2 mg/kg/day.

In some embodiments, the amount of 6TG administered per dose is based on a determination of a patient's HPRT enzyme activity. Those of ordinary skill in the art will appreciate that those presenting with higher levels of HPRT enzyme activity may be provided with doses having lower amounts of a purine analog, such as 6TG. The higher the level of HPRT the greater conversion of the purine analog, such as 6TG, to toxic metabolites. Therefore, the lower dose you would need to administer to achieve the same goal.

Measurement of TPMT genotypes and/or TPMT enzyme activity before instituting 6TG conditioning may identify individuals with low or absent TPMT enzyme activity. As such, in other embodiments, the amount of 6TG administered is based on thiopurine S-methyltransferase (TPMT) levels or TPMT genotype.

In some embodiments, the dosage of a purine analog, such as 6TG, for in vivo chemoselection is administered to the patient one to three times a week on a schedule with a cycle selected from the group consisting of: (i) weekly; (ii) every other week; (iii) one week of therapy followed by two, three or four weeks off; (iv) two weeks of therapy followed by one, two, three or four weeks off; (v) three weeks of therapy followed by one, two, three, four or five weeks off; (vi) four weeks of therapy followed by one, two, three, four or five weeks off; (vii) five weeks of therapy followed by one, two, three, four or five weeks off; and (viii) monthly.

In some embodiments, between about 3 and about 10 dosages of a purine analog, such as 6TG, are administered to the patient over an administration period ranging from 1 week to about 4 weeks. In some embodiments, 4 or 5 dosages of 6TG are administered to the patient over a 14-day period.

5.2 Negative Selection with a Dihydrofolate Reductase Inhibitor

In addition, HPRT-deficient cells can be negatively selected by using a dihydrofolate reductase inhibitor (e.g. MTX) to inhibit the enzyme dihydrofolate reductase (DHFR) in the purine de novo synthetic pathway. This has been developed as a safety procedure to eliminate gene-modified HSCs in case of unexpected adverse effects observed. As such, should any adverse side effects arise, a patient may be treated with a dihydrofolate reductase inhibitor (e.g. MTX or MPA). Adverse side effects include, for example, aberrant blood counts/clonal expansion indicating insertional mutagenesis in a particular clone of cells or cytokine storm.

It is believed that a dihydrofolate reductase inhibitor (e.g. MTX or MPA) competitively inhibits dihydrofolate reductase (DHFR), an enzyme that participates in tetrahydrofolate (THF) synthesis. DHFR catalyzes the conversion of dihydrofolate to active tetrahydrofolate. Folic acid is needed for the de novo synthesis of the nucleoside thymidine, required for DNA synthesis. Also, folate is essential for purine and pyrimidine base biosynthesis, so synthesis will be inhibited. The dihydrofolate reductase inhibitor (e.g. MTX or MPA) therefore inhibits the synthesis of DNA, RNA, thymidylates, and proteins. MTX or MPA blocks the de novo pathway by inhibiting DHFR. In HPRT−/− cell, there is no salvage or de novo pathway functional, leading to no purine synthesis, and therefore the cells die. However, the HPRT wild type cells have a functional salvage pathway, their purine synthesis takes place and the cells survive.

Given the sensitivity of the modified HSCs produced according to the present disclosure, a dihydrofolate reductase inhibitor (e.g. MTX or MPA) may be used to selectively eliminate HPRT-deficient cells. In some embodiments, a dihydrofolate reductase inhibitor (e.g. MTX or MPA) is administered as a single dose. In some embodiments, multiple doses of the dihydrofolate reductase inhibitor are administered.

In some embodiments, an amount of MTX administered ranges from about 2 mg/m2/infusion to about 100 mg/m2/infusion. In some embodiments, an amount of MTX administered ranges from about 2 mg/m2/infusion to about 90 mg/m2/infusion. In some embodiments, an amount of MTX administered ranges from about 2 mg/m2/infusion to about 80 mg/m2/infusion. In some embodiments, an amount of MTX administered ranges from about 2 mg/m2/infusion to about 70 mg/m2/infusion. In some embodiments, an amount of MTX administered ranges from about 2 mg/m2/infusion to about 60 mg/m2/infusion. In some embodiments, an amount of MTX administered ranges from about 2 mg/m2/infusion to about 50 mg/m2/infusion. In some embodiments, an amount of MTX administered ranges from about 2 mg/m2/infusion to about 40 mg/m2/infusion. In some embodiments, an amount of MTX administered ranges from about 2 mg/m2/infusion to about 30 mg/m2/infusion. In some embodiments, an amount of MTX administered ranges from about 20 mg/m2/infusion to about 20 mg/m2/infusion. In some embodiments, an amount of MTX administered ranges from about 2 mg/m2/infusion to about 10 mg/m2/infusion. In some embodiments, an amount of MTX administered ranges from about 2 mg/m2/infusion to about 8 mg/m2/infusion. In other embodiments, an amount of MTX administered ranges from about 2.5 mg/m2/infusion to about 7.5 mg/m2/infusion. In yet other embodiments, an amount of MTX administered is about 5 mg/m2/infusion. In yet further embodiments, an amount of MTX administered is about 7.5 mg/m2/infusion.

In some embodiments, between 2 and 6 infusions are made, and the infusions may each comprise the same dosage or different dosages (e.g. escalating dosages, decreasing dosages, etc.). In some embodiments, the administrations may be made on a weekly basis, or a bi-monthly basis.

In some embodiments, MPA is dosed in an amount of between about 500 mg to about 1500 mg per day. In some embodiments, the dose of MPA is administered in a single bolus. In some embodiments, the dose of MPA is divided into a plurality of individual doses totalling between about 500 mg to about 1500 mg per day.

In some embodiments, an analog or derivative of MTX or MPA may be substituted for MTX or MPA. Derivatives of MTX are described in U.S. Pat. No. 5,958,928 and in PCT Publication No. WO/2007/098089, the disclosures of which are hereby incorporated by reference herein in their entireties. In some embodiments, an alternative agent may be used in place of either MTX or MPA, including, but not limited to ribavarin (IMPDH inhibitor); VX-497 (IMPDH inhibitor) (see Jain J, VX-497: a novel, selective IMPDH inhibitor and immunosuppressive agent, (2001), J Pharm Sci., 90(5):625-37); lometrexol (DDATHF, LY249543) (GAR and/or AICAR inhibitor); thiophene analog (LY254155) (GAR and/or AICAR inhibitor), furan analog (LY222306) (GAR and/or AICAR inhibitor) (see Habeck et al., A Novel Class of Monoglutamated Antifolates Exhibits Tight-binding Inhibition of Human Glycinamide Ribonucleotide Formyltransferase and Potent Activity against Solid Tumors, (1994), Cancer Research, 54, 1021-2026); DACTHF (GAR and/or AICAR inhibitor) (see Cheng et. al. Design, synthesis, and biological evaluation of 10-methanesulfonyl-DDACTHF, 10-methanesulfonyl-5-DACTHF, and 10-methylthio-DDACTHF as potent inhibitors of GAR Tfase and the de novo purine biosynthetic pathway, (2005) Bioorg Med Chem., 13(10):3577-85); AG2034 (GAR and/or AICAR inhibitor) (see Boritzki et. al. AG2034: a novel inhibitor of glycinamide ribonucleotide formyltransferase, (1996), Invest New Drugs., 14(3):295-303); LY309887 (GAR and/or AICAR inhibitor) ((2S)-2-[[5-[2-[(6R)-2-amino-4-oxo-5,6,7,8-tetrahydro-1H-pyrido[2,3-d]pyrimidin-6-yl]ethyl]thiophene-2-carbonyl]amino]pentanedioic acid); alimta (LY231514) (GAR and/or AICAR inhibitor) (see Shih et. al. LY231514, a pyrrolo[2,3-d]pyrimidine-based antifolate that inhibits multiple folate-requiring enzymes, (1997) Cancer Research, 57(6):1116-23); dmAMT (GAR and/or AICAR inhibitor), AG2009 (GAR and/or AICAR inhibitor); forodesine (Immucillin H, BCX-1777; trade names Mundesine and Fodosine) (inhibitor of purine nucleoside phosphorylase [PNP]) (see Kicska et. al., Immucillin H, a powerful transition-state analog inhibitor of purine nucleoside phosphorylase, selectively inhibits human T lymphocytes, (2001) Proceedings Nat'l Acad. Sci. USA, 98 (8) 4593-4598); and immucillin-G (inhibitor of purine nucleoside phosphorylase [PNP]).

### 6. Combination Therapy

In another aspect of the present disclosure is a combination therapy whereby antibacterial, antifungal, and/or antiviral active pharmaceutical ingredients (depending, of course, upon the particular infection presented) are administered prior to, during, or following the administration or transplantation of transduced HSCs (described above) into a patient in need of treatment thereof, e.g. to treat Wiskott-Aldrich Syndrome. In some embodiments, patients with Wiskott-Aldrich Syndrome and having severe thrombocytopenia may be treated with high dose intravenous immunoglobulin (2 gm/kg/day) and/or corticosteroids (2 mg/kg/day) prior to, during, or following the administration or transplantation of transduced HSCs (described above) into a patient in need of treatment thereof. Alternatively, an allogenic transplantation of stem cells from healthy donors may be administered before or after treatment with the expression vectors or transduced stem cells of the present disclosure

### 7. Lentiviral Vectors Useful for the Treatment of Sickle Cell Disease

β-Hemoglobinopathies, including beta-thalassemia and sickle-cell disease (SCD), are a heterogeneous group of commonly inherited disorders affecting the function or levels of hemoglobin. SCD and β-thalassemia major are the most common monogenic disorders in the world with approximately 400,000 affected births each year. Clinical manifestations typically appear several months after birth during the switch from fetal hemoglobin (HbF) to adult β-globin (HbA) and can be severe with substantial morbidity and mortality. Allogenic bone marrow transplantation is curative but limited to those patients with an appropriately matched donor. Autologous gene therapy, which utilizes a patient's own cells, is an attractive therapeutic option.

β-thalassemia is an inherited blood disorder characterized by reduced levels of functional hemoglobin. β-thalassemias are caused by mutations in hemoglobin subunit beta (hereinafter the “HBB gene”), which is believed to be inherited in an autosomal recessive fashion. β-thalassemia major, defined clinically as transfusion-dependent, is caused by reduced or absent synthesis of the beta chain of hemoglobin. The severity of the disease depends on the nature of the mutation with variable outcomes ranging from severe anemia to clinically asymptomatic individuals.

Hundreds of different mutations have been described affecting beta-globin levels via effects on a wide range of processes, including transcription, mRNA splicing/processing, RNA stability, translation, and globin peptide stability. It is believed that the low beta-globin content allows the excess alpha-globin chains to precipitate in erythroid precursors. It is further believed that the alpha-globin aggregates cause cell membrane damage and lead to early erythroid precursor death. The resultant ineffective erythropoiesis found in patients, if severe, may necessitate frequent blood transfusions.

Sickle cell anemia (SCA) results from a single point mutation in Exon 1 of the beta-globin gene leading to the replacement of glutamic acid with valine at position 6 in the mutated sickled form of hemoglobin, hemoglobin S (HbS). There are other genotypes, in addition to homozygous hemoglobin S (“HbSS”), that can result in SCD. While classical SCA is often defined as homozygous HbSS, homozygous hemoglobin C (“HbSC”) and HbS/β⁰ are common genotypes that have essentially the same disease manifestations. HbS polymerizes upon deoxygenation resulting in sickle-shaped red blood cells (“RBCs”) that occlude microvasculature. SCD is characterized clinically by varying degrees of anemia, and episodic vaso-occulsive crisis leading to multi-organ damage and premature death. Besides sickling, excessive hemolysis and a state of chronic inflammation exist.

SCD patients account for approximately 75,000 USA hospitalizations per year, resulting in an estimated annual expenditure of $475 million dollars. Worldwide, SCD is second only to thalassemia in incidence of monogenic disorders, with more than 200,000 children born annually in Africa with this disease. Medical management options currently available for SCD include supportive management of vasoocclusive crisis, long-term transfusions to avoid or prevent recurrence of severe complications of SCD such as stroke or acute chest syndrome, and fetal hemoglobin (HbF) induction with hydroxyurea. A matched allogeneic hematopoietic stem cell (HSC) transplantation is believed to be curative but restricted by the availability of matched related donors and has potential serious complications. In fetal life, the gamma-globin gene (resulting in HbF; alpha₂gamma₂) is the predominant gene expressed by the beta-globin locus and the beta-globin gene expression is repressed. However, after birth, the expression of fetal gamma-globin gene decreases to negligible levels, with a concomitant increase in beta-globin expression. In adult life, fetal gamma-globin transcripts are highly silenced, i.e. gene expression is regulated to prevent or reduce expression of gamma-globin. This change of expression results in decreased HbF with a corresponding increase in HbA (alpha₂beta₂). Gamma-globin is known to have anti-sickling properties and, thus the addition of this gene is considered for gene therapy.

Hemoglobinopathies, especially SCD, are prime targets for gene therapy for a variety of reasons. Their high prevalence, significant morbidity and mortality, and the resulting high cost of lifelong palliative medical care portends that a curative therapy can greatly improve patient outcomes and significantly reduce associated medical costs. Gene therapy for β-hemoglobinopathies by ex vivo lentiviral transfer of a therapeutic β-globin gene into autologous CD34⁺ hematopoietic stem/progenitor cells (HSPC) has been evaluated in human clinical trials. Autologous HSC transplantation based on myeloablative therapy has resulted in transfusion independence or a reduction in transfusion volumes in β-thalassemia patients greater than 12 months after gene therapy.

While clinical trials of gene therapy using viral vectors, such as lentiviral vectors, for the treatment of SCD and other β-hemoglobinopathies have indicated that this approach can be therapeutically effective, there is a continued need to develop vectors with improved efficacy and improved safety profiles.

Accordingly, in another aspect provided herein are lentiviral vectors that contain a modified globin transgene in which SD1 has been inactivated. As would be appreciated, such globin transgenes contain intron 2 derived from β-globin. In particular embodiments, provided are lentiviral vectors that contain a modified γ-globin transgene in which SD1 has been inactivated. Also provided herein are lentiviral vectors that contain a modified HS4-400 insulator in which one or both of SA2 and SA3 has been inactivated. Also provided are vectors that comprise no HS4-400 insulator. The lentiviral vectors of the present disclosure therefore can have associated with them a reduced risk of alternative splicing when introduced into a cell, such as a hematopoietic stem cell.

This aspect of the present disclosure is predicated, a least in part, on the identification of a cryptic splice donor site within intron 2 (which is derived from β-globin) in the γ-globin transgene present in a therapeutic lentiviral vector. This cryptic splice donor site, referred to as SD1, is in the positive strand of the vector in the β-globin intron 2 within the γ-globin transgene. As the γ-globin transgene is present in the vector in the reverse orientation, SD1 is in the complementary strand of the γ-globin transgene, i.e. in the reverse, complement sequence of the—γ-globin transgene. SD1 is located at nucleotides 933-934 of SEQ ID NO:122, where SEQ ID NO:122 is the reverse complement sequence of the γ-globin transgene set forth in SEQ ID NO: 121, i.e. splicing can occur between the G at position 933 and the G at position 934; and nucleotides 150-151 of SEQ ID NO:119, where SEQ ID NO:119 is the reverse complement sequence of the γ-globin transgene set forth in SEQ ID NO:118, i.e. splicing can occur between the G at position 150 and the G at position 150. When referred to in the context of the β-globin intron 2, the cryptic splice donor site is located at nucleotides 20-21 of SEQ ID NO:88, where SEQ ID NO:88 is the reverse complement of the β-globin intron 2 set forth in SEQ ID NO:87, i.e. splicing can occur between the G at position 20 and the G at position 21 of SEQ ID NO:88.

Thus, in an aspect, there is provided a lentiviral vector, comprising: a first promoter operably linked to a first nucleic acid sequence, wherein the first nucleic acid sequence comprises a modified γ-globin transgene comprising a β-globin intron 2; wherein: the modified γ-globin transgene comprises a mutation relative to an unmodified γ-globin transgene, wherein the mutation inactivates splice donor site 1 (SD1) present in an unmodified γ-globin transgene, and wherein:
SD1 is present in an unmodified γ-globin transgene at nucleotide positions 933-934 with numbering relative to SEQ ID NO:122, wherein SEQ ID NO:122 is the reverse, complement sequence of the unmodified γ-globin transgene set forth in SEQ ID NO:121;
SD1 is present in an unmodified γ-globin transgene at nucleotide positions 150-151 with numbering relative to SEQ ID NO:119, wherein SEQ ID NO:119 is the reverse, complement sequence of the unmodified γ-globin transgene set forth in SEQ ID NO:118; and/or
SD1 comprises the sequence AAGATAAGAG\hat{ }GTATGAACAT (SEQ ID NO:96), where \hat{ } represents the splice position.

In some embodiments, the mutation is a mutation of the A at position 932, the G at position 933, the G at position 934 and/or the T at position 935, with numbering relative to SEQ ID NO:122. In particular examples, the mutation is a nucleotide substitution, e.g. a G to A mutation at position 934, with numbering relative to SEQ ID NO:122. In some examples, the modified γ-globin transgene comprises the sequence set forth in SEQ ID NO:91.

In some embodiments, the lentiviral vector further comprises a modified HS4-400 insulator.

In one example, the modified HS4-400 insulator, when present in the vector, comprises an inactivated splice acceptor site 2 (SA2) relative to an unmodified HS4-400 insulator, and wherein: SA2 is present in an unmodified HS4-400 insulator at nucleotide positions 190-191, with numbering relative to SEQ ID NO:90, wherein SEQ ID NO:90 is the reverse, complement sequence of the unmodified HS4-400 insulator set forth in SEQ ID NO:89; and/or SA2 comprises the sequence ATCCCCCCAG\hat{ }GTGTCTGCAG (SEQ ID NO:61), where \hat{ } represents the splice position. In some examples, the modified HS4-400 insulator comprises, relative to an unmodified HS4-400 insulator, a mutation that inactivates SA2, such as a mutation of the A at position 189 (e.g. an A to T mutation), the G at position 190, the G at position 191, and/or the T at position 192, with numbering relative to SEQ ID NO:90. In some examples, the reverse complement sequence of the modified HS4-400 insulator comprises the sequence set forth in SEQ ID NO:93.

In another example, the modified HS4-400 insulator, when present in the lentiviral vector, comprises a mutation that inactivates splice acceptor site 3 (SA3) relative to an unmodified HS4-400 insulator, wherein: SA3 is present in an unmodified HS4-400 insulator at nucleotide positions 200-201, with numbering relative to SEQ ID NO:90; and/or wherein SA3 comprises the sequence GTGTCTGCAG\hat{ }GCTCAAAGAG (SEQ ID NO:62), where \hat{ } represents the splice position. In some examples, the mutation is a mutation of the A at position 199 (e.g. an A to T mutation), the G at position 200, the G at position 201, and/or the C at position 202, with numbering relative to SEQ ID NO:90. In particular embodiments, the reverse complement sequence of the modified HS4-400 insulator comprises the sequence set forth in any one of SEQ ID NOs:94-95.

In some examples, the modified HS4-400 insulator is in the reverse orientation within the lentiviral vector. In a particular embodiment, the first nucleic acid is in the reverse orientation and the modified HS4-400 insulator is in the reverse orientation within the lentiviral vector.

In other example, the modified HS4-400 is in the forward orientation within the lentiviral vector.

In another aspect, provided is a lentiviral vector, comprising:
a first promoter a first promoter operably linked to a first nucleic acid sequence, wherein the first nucleic acid sequence comprises a modified γ-globin transgene comprising a β-globin intron 2; and
a modified HS4-400 insulator, wherein:
when present in the vector, the modified HS4-400 insulator comprises an inactivated splice acceptor site 2 (SA2) relative to an unmodified HS4-400 insulator, and wherein:
   SA2 is present in an unmodified HS4-400 insulator at nucleotide positions 190-191, with numbering relative to SEQ ID NO:90, wherein SEQ ID NO:90 is the reverse, complement sequence of the unmodified HS4-400 insulator set forth in SEQ ID NO:89; and/or
   SA2 comprises the sequence ATCCCCCCAG\hat{ }GTGTCTGCAG (SEQ ID NO:61), where \hat{ } represents the splice position.

In one embodiment, the modified HS4-400 insulator comprises, relative to an unmodified HS4-400 insulator, a mutation that inactivates SA2, such as a mutation of the A at position 189 (e.g. an A to T mutation), the G at position 190, the G at position 191, and/or the T at position 192, with numbering relative to SEQ ID NO:90. In particular examples, the reverse complement sequence of the modified HS4-400 insulator comprises the sequence set forth in SEQ ID NO:93.

In a further embodiment, the modified HS4-400 insulator further comprises a mutation that inactivates splice acceptor site 3 (SA3) relative to an unmodified HS4-400 insulator, wherein: SA3 is present in an unmodified HS4-400 insulator at nucleotide positions 200-201, with numbering relative to SEQ ID NO:90; and/or wherein SA3 comprises the sequence GTGTCTGCAG\hat{ }GCTCAAAGAG (SEQ ID NO:62), where \hat{ } represents the splice position. In some examples, the mutation is a mutation of the A at position 199 (e.g. an A to T mutation), the G at position 200, the G at position 201, and/or the C at position 202, with numbering relative to SEQ ID NO:90. In particular embodiments, the reverse complement sequence of the modified HS4-400 insulator comprises the sequence set forth in SEQ ID NO:94.

In one example of this aspect, the modified HS4-400 insulator is in the reverse orientation within the lentiviral vector. In another example, the modified HS4-400 insulator is in the forward orientation within the lentiviral vector, thereby inactivating SA2.

In another aspect, provided is a lentiviral vector, comprising:
a first promoter a first promoter operably linked to a first nucleic acid sequence, wherein the first nucleic acid sequence comprises a modified γ-globin transgene comprising a β-globin intron 2; and
a modified HS4-400 insulator, wherein:
when present in the vector, the modified HS4-400 insulator comprises an inactivated splice acceptor site 3 (SA3) relative to an unmodified HS4-400 insulator, and wherein:
   SA3 is present in an unmodified HS4-400 insulator at nucleotide positions 200-201, with numbering relative to SEQ ID NO:90, wherein SEQ ID NO:90 is the reverse, complement sequence of the unmodified HS4-400 insulator set forth in SEQ ID NO:89; and/or
   SA3 comprises the sequence GTGTCTGCAG\hat{ }GCTCAAAGAG (SEQ ID NO:62), where \hat{ } represents the splice position.

In one embodiment, the modified HS4-400 insulator comprises, relative to an unmodified HS4-400 insulator, a mutation that inactivates SA3, e.g. a mutation of the A at position 199 (e.g. an A to T mutation), the G at position 200, the G at position 201, and/or the C at position 202, with numbering relative to SEQ ID NO:90. In some embodiments, the modified HS4-400 insulator comprises the sequence set forth in SEQ ID NO:95.

In some examples, the modified HS4-400 insulator further comprises a mutation that inactivates splice acceptor site 2 (SA2) relative to an unmodified HS4-400 insulator, and wherein: SA2 is present in an unmodified HS4-400 insulator at nucleotide positions 190-191, with numbering relative to SEQ ID NO:90, wherein SEQ ID NO:90 is the reverse, complement sequence of the unmodified HS4-400 insulator set forth in SEQ ID NO:89; and/or SA2 comprises the sequence ATCCCCCCAG\hat{ }GTGTCTGCAG (SEQ ID NO:61), where \hat{ } represents the splice position. In some examples, the mutation is a mutation of the A at position 189 (e.g. an A to T mutation), the G at position 190, the G at position 191, and/or the T at position 192, with numbering relative to SEQ ID NO:90. In particular embodiments, the modified HS4-400 insulator comprises the sequence set forth in SEQ ID NO:94.

In some examples of this aspect, the modified HS4-400 insulator is in the reverse orientation within the lentiviral vector. In other examples, the modified HS4-400 insulator is in the forward orientation within the lentiviral vector, thereby inactivating SA3.

In some embodiments, the unmodified γ-globin transgene comprises the sequence set forth in SEQ ID NO:85 or a sequence having at least 90%, 91%, 92%, 93%, 94%, 95%, 96%, 97%, 98% or 99% sequence identity thereto. In one example, the modified γ-globin transgene encodes a γ-globin comprising an amino acid sequence set forth in SEQ ID NO:103 or a sequence having at least 90%, 91%, 92%, 93%, 94%, 95%, 96%, 97%, 98% or 99% sequence identity thereto.

In some examples, the transgene comprises just the γ-globin coding sequence (e.g. as set forth in SEQ ID NO:101 or 102). In particular examples, the γ-globin transgene comprises exons and introns and are associated with other non-coding elements. In one example, the γ-globin transgene comprises γ-globin exon 1 (or HBG exon 1, e.g. as set forth in SEQ ID NO:98), γ-globin exon 2 (or HBG exon 2, e.g. as set forth in SEQ ID NO:99), and γ-globin exon 3 (or HBG exon 3, e.g. as set forth in SEQ ID NO:100). The introns may include γ-globin intron 1 (or HBG intron 1), and a β-globin intron 2 (HBB intron 2, such as a truncated HBB intron 2, e.g. as set forth in SEQ ID NO:87). In a particular embodiment, the transgene comprises the sequence set forth in SEQ ID NO:118 (i.e. HBG exon 1, HBG intron 1, HBG exon 2, HBB truncated intron 2, and HBG exon 3) or SEQ ID NO:121 (i.e. HBG exon 1, HBG intron 1, HBG exon 2, HBB truncated intron 2, HBG exon 3 and 3′UTR/polyA signal). The transgene can optionally be associated with other non-coding elements such as a β-globin Locus control region (LCR) (e.g. as set forth in SEQ ID NO: 105).

As determined herein, γ-globin transgenes that contain a β-globin intron 2 may have a cryptic splice donor site (SD1) when in the lentiviral vector. This splice donor site was identified in the γ-globin transgenes set forth in SEQ ID NOs:118 and 121 when the transgene was present in a lentiviral vector in the reverse orientation, whereby SD1 was in β-globin intron 2 in the positive strand of the vector. Thus, SD1 was in the reverse complement sequence of SEQ ID NOs:118 and 121. These reverse complement sequences are set forth as SEQ ID NOs:120 and 122.

SD1 is present at position 933-934 of SEQ ID NO:121 (i.e. splicing occurs between the G at position 933 and the G at position 934) and at position 150-151 of SEQ ID NO:119 (i.e. splicing can occur between the G at position 150 and the G at position 150) and corresponding positions of other γ-globin transgenes that contain a β-globin intron 2. SD1 can also be defined as comprising the sequence AAGATAAGAG\hat{ }GTATGAACAT (SEQ ID NO:96), where \hat{ } represents the splice position; or comprising the sequence of nucleotides at positions 924-943 of the complementary strand of γ-globin transgene that contains a β-globin intron 2, with numbering relative to SEQ ID NO:121; or comprising the sequence of nucleotides at positions 141-160 of the complementary strand of γ-globin transgene that contains a β-globin intron 2, with numbering relative to SEQ ID NO:119. When considered in the context of β-globin intron 2, SD1 is present at positions 20-21 of SEQ ID NO:88 (i.e. splicing occurs between the G at position 20 and the G at position 21) and corresponding positions of other β-globin intron 2. SD1 can therefore also be defined as comprising the sequence of nucleotides at positions 11-30 of the complementary strand of β-globin intron 2, with numbering relative to SEQ ID NO:88.

Thus, in some embodiments, the lentiviral vectors of the present disclosure comprise a first promoter operably linked to a first nucleic acid sequence, wherein the first nucleic acid sequence comprises a modified γ-globin transgene comprising a β-globin intron 2; wherein the modified γ-globin transgene comprises a mutation relative to an unmodified γ-globin transgene, wherein the mutation inactivates SD1. A lentiviral vector comprising the modified γ-globin transgene can exhibit reduced splicing at position 924-943 or 150-151 when transduced into a cell compared to the splicing that occurs at position 924-943 or 150-151 with a lentiviral vector that comprises an unmodified γ-globin transgene, with numbering relative to SEQ ID NO:122 or 119, respectively. In some examples, splicing is reduced by at least or about 20%, 30%, 40%, 50%, 60%, 70%, 80% or 90%.

Unmodified γ-globin transgenes include those that, when present in a lentiviral vector, comprise an active SD1, i.e. comprise a sequence and orientation within the lentiviral vector that can facilitate splicing at SD1. Exemplary unmodified γ-globin transgene include those that encode a γ-globin and that comprise a sequence set forth in SEQ ID NO:118 and 121 (with reverse complement sequences set forth in SEQ ID NO:119 and 122, respectively) and sequences having at least or about 80%, 81%, 82%, 83%, 84%, 85%, 86%, 87%, 88%, 89%, 90%, 91%, 92%, 93%, 94%, 95%, 96%, 97%, 98% or 99% sequence identity thereto (provided the SD1 site is still present, e.g. provided the reverse complement of the γ-globin transgene comprises the sequence AAGATAAGAGGTATGAACAT (SEQ ID NO:96)).

In particular examples, the modified γ-globin transgene contains a mutation (e.g. a nucleotide deletion, insertion or replacement) relative to an unmodified γ-globin transgene, wherein the mutation inactivates SD1 that is present in the unmodified γ-globin transgene (or reduces splicing at position 924-943 of the reverse complement sequence of the modified γ-globin transgene compared to the splicing that occurs at position 924-943 of the reverse complement sequence of an unmodified γ-globin transgene, with numbering relative to SEQ ID NO:122). The mutation can be any that inactivates or disrupts SD1. In some examples, the mutation is a deletion or substitution of any nucleotide in the SD1 sequence or a nucleotide insertion into the SD1 sequence (e.g. the sequence AAGATAAGAGGTATGAACAT (SEQ ID NO:96)). In particular examples, the mutation is a mutation (e.g. deletion or substitution) of the A at position 932, the G at position 933, the G at position 934 and/or the T at position 935, with numbering relative to SEQ ID NO: 122 (e.g., is a mutation at A at position 149, the G at position 150, the G at position 151 and/or the T at position 152, with numbering relative to SEQ ID NO:119). For example, the modified γ-globin transgene can comprise an A to T, A to C or A to G mutation at position 932, a G to C, G to A or G to T mutation at position 933, an G to C, G to T or G to A mutation at position 934, and/or a T to A, T to C or T to G mutation at position 935, with numbering relative to SEQ ID NO: 122 (i.e., an A to T, A to C or A to G mutation at position 149, a G to C, G to A or G to T mutation at position 150, an G to C, G to T or G to A mutation at position 151, and/or a T to A, T to C or T to G mutation at position 152, with numbering relative to SEQ ID NO:119). In other examples, the mutation comprises an insertion of a nucleotide after position 932, 933 and/or 934, with numbering relative to SEQ ID NO:122 (i.e. an insertion of a nucleotide after position 149, 150 and/or 151, with numbering relative to SEQ ID NO:119. In some examples, the modified γ-globin transgene comprises two or more of such mutations.

In one example, the modified γ-globin transgene comprises a G to A mutation in the reverse complement sequence at position 934, with numbering relative to SEQ ID NO:122 (i.e. comprises an A at position 934, with numbering relative to SEQ ID NO:122). Thus, in some examples, the modified γ-globin transgene comprises a G to A mutation in the reverse complement sequence at position 151, with numbering relative to SEQ ID NO:119 (i.e. comprises an A at position 151, with numbering relative to SEQ ID NO:119). In particular embodiments, the reverse complement sequence of the γ-globin transgene comprises the sequence set forth in SEQ ID NO:123 or a sequence having at least or about 90%, 91%, 92%, 93%, 94%, 95%, 96%, 97%, 98% or 99% sequence identity thereto (provided there is an A at position 934, with numbering relative to SEQ ID NO:122). In other embodiments, the reverse complement sequence of the γ-globin transgene comprises the sequence set forth in SEQ ID NO:120 or a sequence having at least or about 90%, 91%, 92%, 93%, 94%, 95%, 96%, 97%, 98% or 99% sequence identity thereto (provided there is an A at position 151, with numbering relative to SEQ ID NO:119).

In some examples, the modified γ-globin transgene described herein having a mutation that inactivates SD1 is in the reverse orientation within the lentiviral vector.

In further embodiments, the first promoter is a β-globin promoter, such as one comprising the nucleic acid sequence set forth in any one of SEQ ID NOs:115-117 or a sequence having at least 90%, 91%, 92%, 93%, 94%, 95%, 96%, 97%, 98% or 99% sequence identity thereto.

In an embodiment, the first promoter is a β-globin promoter and is operably linked to a first nucleic acid comprising the γ-globin transgene.

In some embodiments, the lentiviral vectors further comprise a second promoter operably linked to a second nucleic acid sequence, wherein the second nucleic acid sequence encodes a nucleic acid that inhibits HPRT expression. In some examples, the nucleic acid that inhibits HPRT expression is a shRNA, e.g. an shRNA that comprises a hairpin loop sequence set forth in of SEQ ID NO:66 and/or that comprises a nucleic acid sequence set forth in any one of SEQ ID NOs:67-68, or a sequence comprising at least 95% sequence identity thereto. In one example, the second promoter comprises a Pol III promoter or a Pol II promoter, such as one comprising 7sk (e.g. one comprising a nucleic acid sequence set forth in any one of SEQ ID NOs:69-71 or a sequence having at least 95% sequence identity thereto). In a particular embodiment, the second promoter and the operably linked second nucleic acid sequence are in the forward orientation and downstream of the first promoter and the operably linked first nucleic acid, which are in the reverse orientation.

In some examples, the lentiviral vectors further comprise a polyadenylation signal in the 3′ LTR of the vector. The polyadenylation signal may be, for example, a rabbit β-globin polyadenylation signal comprising a nucleic acid sequence set forth in SEQ ID NO:103 or a sequence having at least 95% sequence identity thereto.

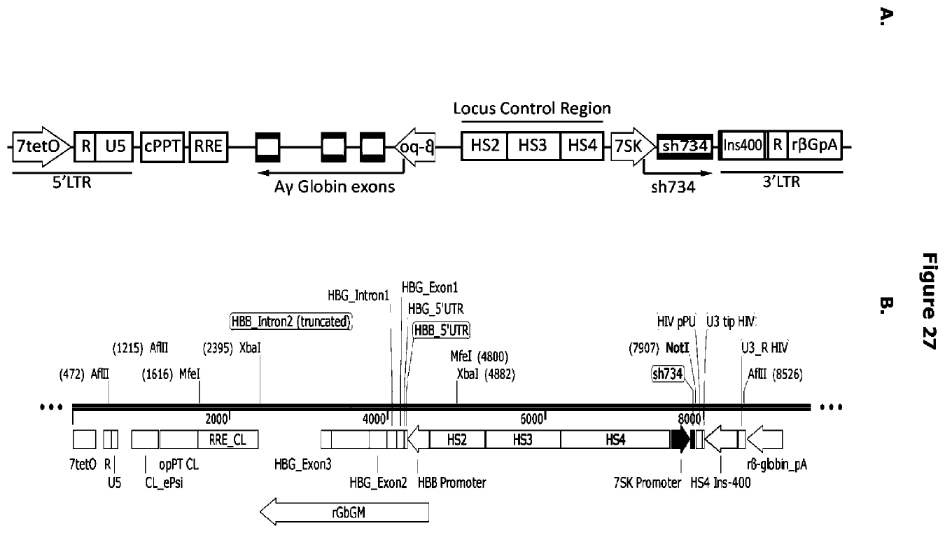

In another aspect of the present disclosure there is provided lentiviral vectors including nucleic acid vectors (e.g. plasmids) and lentivirus virions (or virus particles) that comprise a 5′LTR (including a 7tetO promoter/operator, R and U5, such as shown schematically in FIG. 27) downstream of which, from 5′ to 3′, is a central polypurine tract (cPPT), a REV response element (RRE) (such as one comprising the sequence set forth in SEQ ID NO:106 or a sequence having at least 85%, 86%, 87%, 88%, 89%, 90%, 91%, 92%, 93%, 94%, 95%, 96%, 97% or 99% sequence identity thereto), a γ-globin expression cassette comprising a β-globin promoter (e.g. one comprising the sequence set forth in any one of SEQ ID NOs:115-117 or a sequence having at least 85%, 86%, 87%, 88%, 89%, 90%, 91%, 92%, 93%, 94%, 95%, 96%, 97% or 99% sequence identity thereto) operably linked to a γ-globin transgene (such as a modified γ-globin transgene described herein having an inactivated SD1, e.g. one comprising a complementary strand comprising the sequence set forth in SEQ ID NO:123 or a sequence having at least 85%, 86%, 87%, 88%, 89%, 90%, 91%, 92%, 93%, 94%, 95%, 96%, 97% or 99% sequence identity thereto wherein the sequence comprises an A at position 934 with numbering relative to SEQ ID NO:122, or one comprising a complementary strand comprising the sequence set forth in SEQ ID NO:120 or a sequence having at least 85%, 86%, 87%, 88%, 89%, 90%, 91%, 92%, 93%, 94%, 95%, 96%, 97% or 99% sequence identity thereto, wherein the sequence comprises an A at position 151 with numbering relative to SEQ ID NO:119), a β-globin LCR (e.g. one comprising the sequence set forth in SEQ ID NO:105 or a sequence having at least 85%, 86%, 87%, 88%, 89%, 90%, 91%, 92%, 93%, 94%, 95%, 96%, 97% or 99% sequence identity thereto), a 7sk-sh734 expression cassette comprising a 7sk promoter operably linked to nucleic acid encoding sh734, and a 3′LTR, which includes a HS4-400 insulator (such as a modified HS4-400 insulator described herein having an inactivated SA2 and/or SA3, e.g. one comprising a complementary strand comprising the sequence set forth in SEQ ID NO:93 or a sequence having at least 85%, 86%, 87%, 88%, 89%, 90%, 91%, 92%, 93%, 94%, 95%, 96%, 97% or 99% sequence identity thereto, wherein the sequence comprises a T at position 189 with numbering relative to SEQ ID NO:93; one comprising a complementary strand comprising the sequence set forth in SEQ ID NO:95 or a sequence having at least 85%, 86%, 87%, 88%, 89%, 90%, 91%, 92%, 93%, 94%, 95%, 96%, 97% or 99% sequence identity thereto, wherein the sequence comprises a T at position 199 with numbering relative to SEQ ID NO:95; or one comprising a complementary strand comprising the sequence set forth in SEQ ID NO:94 or a sequence having at least 85%, 86%, 87%, 88%, 89%, 90%, 91%, 92%, 93%, 94%, 95%, 96%, 97% or 99% sequence identity thereto, wherein the sequence comprises a T at position 189 and a T at position 199 with numbering relative to SEQ ID NO:94), R and a β-globin poly(A) signal (e.g. one comprising the sequence set forth in SEQ ID NO:104 or a sequence having at least 85%, 86%, 87%, 88%, 89%, 90%, 91%, 92%, 93%, 94%, 95%, 96%, 97% or 99% sequence identity thereto). In some examples, the γ-globin expression cassette is one in which the complementary strand comprises the sequence set forth in SEQ ID NO:91 or comprising a sequence having at least 85%, 86%, 87%, 88%, 89%, 90%, 91%, 92%, 93%, 94%, 95%, 96%, 97% or 99% sequence identity thereto wherein the sequence comprises an A at position 934 with numbering relative to SEQ ID NO:86. In these vectors, typically the γ-globin expression cassette is in the reverse orientation and the 7sk-sh734 expression cassette is in the forward orientation.

In some embodiments, the lentiviral vectors are plasmid. In other embodiments, the lentiviral vectors are viral particles.

In one embodiment, the vector is a plasmid and comprises the sequence set forth in SEQ ID NO:109 or a sequence having at least 85%, 86%, 87%, 88%, 89%, 90%, 91%, 92%, 93%, 94%, 95%, 96%, 97% or 99% sequence identity thereto, provided the vector comprises an A at position 934 of the γ-globin expression cassette with numbering relative to SEQ ID NO:91).

In another embodiment, the vector is a plasmid and comprises the sequence set forth in SEQ ID NO:110 or a sequence having at least 85%, 86%, 87%, 88%, 89%, 90%, 91%, 92%, 93%, 94%, 95%, 96%, 97% or 99% sequence identity thereto, provided the vector comprises a T at position 189 and a T at position 199 of the HS4-400 insulator with numbering relative to SEQ ID NO:94.

In another embodiment, the vector is a plasmid and comprises the sequence set forth in SEQ ID NO:111 or a sequence having at least 85%, 86%, 87%, 88%, 89%, 90%, 91%, 92%, 93%, 94%, 95%, 96%, 97% or 99% sequence identity thereto, provided the vector comprises an A at position 934 of the γ-globin expression cassette with numbering relative to SEQ ID NO:91 and provided the vector comprises a T at position 189 and a T at position 199 of the HS4-400 insulator with numbering relative to SEQ ID NO:94.

In one embodiment, the vector is a plasmid and comprises the sequence set forth in SEQ ID NO:112 or a sequence having at least 85%, 86%, 87%, 88%, 89%, 90%, 91%, 92%, 93%, 94%, 95%, 96%, 97% or 99% sequence identity thereto, provided the vector comprises an A at position 934 of the γ-globin expression cassette with numbering relative to SEQ ID NO:91.

In another embodiment, the vector is a plasmid and comprises the sequence set forth in SEQ ID NO:113 or a sequence having at least 85%, 86%, 87%, 88%, 89%, 90%, 91%, 92%, 93%, 94%, 95%, 96%, 97% or 99% sequence identity thereto, provided the vector comprises a T at position 189 of the HS4-400 insulator with numbering relative to SEQ ID NO:93.

In another embodiment, the vector is a plasmid and comprises the sequence set forth in SEQ ID NO:114 or a sequence having at least 85%, 86%, 87%, 88%, 89%, 90%, 91%, 92%, 93%, 94%, 95%, 96%, 97% or 99% sequence identity thereto, provided the vector comprises an A at position 934 of the γ-globin expression cassette with numbering relative to SEQ ID NO:91 and provided the vector comprises a T at position 189 of the HS4-400 insulator with numbering relative to SEQ ID NO:93.

Also provided are host cells, comprising or transduced with a lentiviral vector of the present disclosure. In some examples, the host cell is a hematopoietic stem cell (HSC) (e.g. an allogeneic or autologous HSC). In example, the host cell is HPRT-deficient.

In a further aspect, provided is method of treating a subject with Sickle Cell Disease or β-thalassemia, comprising administering to the subject the host cell described above and herein. In one embodiment, the method comprises administering to the subject the host cell and then administering a purine analog (e.g. 6-thioguanine (“6TG”), 6-mercaptopurine (“6MP”) or azathiopurine (“AZA”)) to the subject to increase engraftment of the host cell. In some examples, the method further comprises pre-conditioning the subject with a purine analog prior to administering the host cell. Also provided are uses of the host cell for the preparation of a medicament for the treatment of Sickle Cell Disease or 3-thalassemia.

It is believed that genetic correction of HSCs with a vector encoding the gamma globin gene would result in a continuous (i.e. permanent) production of the anti-sickling HbF, thereby preventing or mitigating RBC sickling for the life of the subject. It is believed that this method has advantages over currently available therapies, including its availability to all patients, particularly those who do not have a matched sibling donor, and the fact that it would be a one-time treatment, resulting in lifelong correction. It is also believed that the method is advantageously devoid of any immune side effects. It is further believed that an effective gene therapy approach will revolutionize the way SCD is treated and improve the outcomes of patients with this devastating disorder.

As noted herein, in addition to the γ-globin transgene, the vectors of the present disclosure may include an agent designed to inhibit or knockdown HPRT expression (e.g. a shRNA, and hence provide for an in vivo chemoselection strategy that exploits the essential role that HPRT plays in metabolizing purine analogs, e.g. 6TG, into myelotoxic agents. Because HPRT-deficiency does not impair hematopoietic cell development or function, it can be removed from hematopoietic cells used for transplantation. Conditioning and chemoselection with a purine analog is discussed further herein.

In the context of the treatment of sickle cell anemia or β-thalassemia, the treatment of a subject includes the steps of identifying a subject in need of treatment thereof; transfecting hematopoietic stem cells (HSCs) (e.g. autologous HSCs) with a vector (e.g. a lentiviral vector) of the present disclosure (i.e. a vector comprising the mutated human gamma-globin gene and a shRNA to HPRT); and transplanting the transfected HSCs into the subject.

In some embodiments, the method of treating hemoglobinopathies comprises (i) transducing HSCs with a vector comprising at least two nucleic acid sequences, namely a nucleic acid sequence encoding a shRNA to the HPRT gene, and a nucleic acid sequence encoding a gamma globin gene, and (ii) administering the transduced HSCs to a mammalian subject. In some embodiments, the method further comprises a step of myeloablative conditioning prior to the administration of the transduced HSCs. In some embodiments, the method further comprises the step of in vivo chemoselection utilizing a purine analog (e.g. 6TG) following administration of the transduced HSCs. In some embodiments, the method further comprises the step of negative selection utilizing MTX or MTA.

In some embodiments, post-transplantation fetal hemoglobin exceeds at least 20%; F cells constitute at least ⅔ of the circulating red blood cells; fetal hemoglobin per F cells account for at least ⅓ of total hemoglobin in sickle red blood cells; and at least 20% gene-modified HSCs re-populate bone marrow of the subject. In some embodiments, post-transplantation fetal hemoglobin exceeds 25%, 30%, 35%, 40%, 45%, 50%, or greater. In some embodiments, post-transplantation fetal hemoglobin exceeds 55%, 60%, 65%, 70%, 75%, 80%, 85%, 90%, 95%, or greater. In some embodiments, F cells constitute at least 70%, 75%, 80%, 85%, 90%, 95%, or greater of the circulating red blood cells. In some embodiments, fetal hemoglobin per F cells account for at least ⅓ of total hemoglobin in sickle red blood cells. In some embodiments, fetal hemoglobin per F cells account for at least 40%, 45%, 50%, 55%, 60%, 65%, 70%, 75%, 80%, 85%, 90%, 95% or greater of total hemoglobin in sickle red blood cells. In some embodiments, 20%, 25%, 30%, 35%, 40%, 45%, 50%, 55%, 60%, 65%, 70%, 75%, 80%, 85%, 90%, 95%, or greater gene-modified HSCs re-populate bone marrow of the subject.

In another aspect of the present disclosure is a method of treating treat immune deficiencies, hereditary diseases, blood diseases (e.g. hemophilia, hemoglobin disorders), lysosomal storage diseases, neurological diseases, angiogenic disorders, or cancer comprising administering an effective amount of a vector to a mammalian subject, the vector comprising at least two nucleic acid sequences, namely a nucleic acid sequence encoding an RNAi to the HPRT gene, and a nucleic acid sequence encoding a therapeutic gene.

In another aspect of the present disclosure is a method of treating hemoglobinopathies comprising administering an effective amount of a vector to a mammalian subject, the vector comprising at least two nucleic acid sequences, namely a nucleic acid sequence encoding an RNAi to knockout or otherwise decrease the expression of the HPRT gene, and a nucleic acid sequence encoding a gamma globin gene. In some embodiments, the method comprises administering an effective amount of a pharmaceutical composition to a patient, the pharmaceutical composition comprising (i) a vector comprising at least two nucleic acid sequences, namely a nucleic acid sequence encoding a shRNA to the HPRT gene, and a nucleic acid sequence encoding a gamma globin gene, and (ii) a pharmaceutically acceptable carrier. In some embodiments, the method further comprises a step of myeloablative conditioning prior to the administration of the transduced HSCs. In some embodiments, the method further comprises the step of in vivo chemoselection utilizing 6TG following administration of the transduced HSCs. In some embodiments, the method further comprises the step of negative selection utilizing MTX.

In order that the invention may be readily understood and put into practical effect, particular preferred embodiments will now be described by way of the following non-limiting example.

### EXAMPLES

### Example 1

### Identification of Cryptic Splice Sites in was LVV

A lentiviral vector containing WAS cDNA was assessed for cryptic splice sites. This lentiviral vector is the plasmid pBRNGTR47_pTL20c_SK734rev_MND_WAS_650 (or pBRNGTR47) having a sequence set forth in SEQ ID NO:55. As can be seen from FIG. 1, pBRNGTR47 contains a first expression cassette in the forward orientation containing WAS cDNA under the control of a MND promoter. Downstream of the WAS cDNA is a Woodchuck Hepatitis Virus (WHV) Posttranscriptional Regulatory Element (WPRE) followed by a HS4-650 insulator (in the reverse orientation) and β-globin polyA signal. A second expression cassette, which is upstream of the first expression construct and in the reverse orientation, includes nucleic acid encoding shRNA 734 under the control of a 7sk promoter. Lentiviral DNA (including the viral genes and LTR elements) are under the control of a 7tetO promoter/operator (see FIG. 1). The positions and orientation of each of these elements within vector is provided in Table 4 below.

TABLE 4

| | | Start (nt | End (nt |
|---|---|---|---|
| Element | Orientation | position) | position) |
| 7tetO promoter | forward | 28 | 315 |
| shRNA 734 | reverse | 2402 | 2448 |
| 7SK promoter | reverse | 2449 | 2697 |
| MND promoter | forward | 2710 | 3056 |
| WAS cDNA (wild-type ORF) | forward | 3098 | 4606 |
| WPRE | forward | 4615 | 5204 |
| cHS4-650 insulator | reverse | 5340 | 6004 |
| rabbit beta-globin | forward | 6131 | 6579 |
| polyadenylation signal | | | |

Bioinformatic splice site prediction analysis (Netgene2) was used to identify potential splice sites in pBRNGTR47. Three key splice acceptor sites (splice acceptor site 1 (SA1), splice acceptor site 2 (SA2), splice acceptor site 3 (SA3) were identified HS4-650 insulator on the positive strand of the vector with levels of confidence ranging from 0.30 to 0.82 (see FIG. 2). As the HS4-650 insulator is in the reverse orientation in pBRNGTR47, the splice acceptor sites are in the reverse complement sequence of the HS4-650 insulator. These three sites were considered particularly prone to the induction of aberrant transcripts. Table 5 below sets forth the details of SA1, SA2 and SA3.

TABLE 5

| Splice | pos | | | Sequence of splice |
|---|---|---|---|---|
| site | 5′->3′ | | Confidence | site in vector |
| Ref | in vector | strand | score | 5′ intron^{∧}exon 3′ |
| SA1 | 5724 | + | 0.82 | TTGCATCCAG^{∧}ACACCATCAA |
| | | | | (SEQ ID NO: 60) |
| SA2 | 5785 | + | 0.77 | ATCCCCCCAG^{∧}GTGTCTGCAG |
| | | | | (SEQ ID NO: 61) |
| SA3 | 5795 | + | 0.30 | GTGTCTGCAG^{∧}GCTCAAAGAG |
| | | | | (SEQ ID NO: 62) |

Position: Nucleotide position number is of the G immediately 5′ of the site of splicing in SEQ ID NO: 55.
Strand: The strand in which is located the splice site; (+) = forward (−) = reverse
Confidence Score: Confidence value provided by NetGene software that estimates the probability that a given sequence is a true splice site (1 = maximum value; for splice donors (SD) a score >0.5 is considered significant; for splice acceptors (SA) a score of >0.2 is considered significant).
^{∧}A denotes splice site

### Example 2

Generation of Vectors with Inactivated Splice Acceptor Sites

A series of modified vectors was generated to inactivate SA1, SA2 and/or SA3. These vectors contain either a mutation in the splice acceptor site, or an inversion of the HS4-650 insulator, such that it is present within the vector in the forward orientation (similar to the WAS cDNA), thereby placing the splice acceptor site sequences on the reverse strand. For those vectors that contained a mutation in the HS4-650 insulator to inactivate a splice acceptor site, the mutation was an A to T mutation, as shown below in Table 6.

TABLE 6

| | | Sequence of inactivated | |
|---|---|---|---|
| Splice | Sequence of splice site | splice site in modified | |
| site | in pBRNGTR47 vector | vector (mutation | Modified vectors |
| Ref | 5′ intron^{∧}exon 3′ | underlined) | with inactivation |
| SA1 | TTGCATCCAG^{∧}ACACCATCAA | TTGCATCCTG^{∧}ACACCATCAA | pBRNGTR87, |
| | (SEQ ID NO: 60) | (SEQ ID NO: 63) | pBRNGTR88, |
| | | | pBRNGTR119, |
| | | | pBRNGTR120 |
| SA2 | ATCCCCCCAG^{∧}GTGTCTGCAG | ATCCCCCCTG^{∧}GTGTCTGCAG | pBRNGTR87, |
| | (SEQ ID NO: 61) | (SEQ ID NO: 64) | pBRNGTR88, |
| | | | pBRNGTR119, |
| | | | pBRNGTR120 |
| SA3 | GTGTCTGCAG^{∧}GCTCAAAGAG | GTGTCTGCTG^{∧}GCTCAAAGAG | pBRNGTR119, |
| | (SEQ ID NO: 62) | (SEQ ID NO: 65) | pBRNGTR120 |

Table 7 summarizes the vectors produced. Some vectors (pBRNGTR83, pBRNGTR87, pBRNGTR91 and pBRNGTR119) lack the second expression cassette (i.e. the p7sk-shRNA 734 expression cassette). All vectors include WPRE downstream of the WAS cDNA, although the sequence varies, with the WPRE in pBRNGTR47 including 7 mutations (WPRE mut7) when compared to the wild-type sequence, and the newly-generated vectors utilizing a WPRE with 6 mutations (mut6) when compared to the wild-type sequence (also referred to in literature as WPRE mut6). All newly-generated vectors also include an additional 2 bp in the U3 sequence upstream of the insulator. This had been deleted in pBRNGTR47 but is reintroduced in pBRNGTR83, pBRNGTR84, pBRNGTR87, pBRNGTR88, pBRNGTR91, pBRNGTR92, pBRNGTR119 and pBRNGTR120). The vectors having 2 point mutations to inactivate SA1 and SA2 include pBRNGTR87 and pBRNGTR88, and the vectors having 3 point mutations to inactivate SA1, SA2 and SA3 include pBRNGTR119 and pBRNGTR120. The vectors having an inversion of the HS4-650 insulator so as to inactivate the splice sites include pBRNGTR91 and pBRNGTR92.

TABLE 7

| | p7sk-shRNA | | | SEQ |
|---|---|---|---|---|
| Vector | 734 cassette | WPRE | Insulator | ID NO: |
| pBRNGTR47 | Yes | mut7 | Original HS4-650 insulator, | 55 |
| (control) | | | with splice sites, in reverse | |
| | | | orientation | |
| pBRNGTR83 | No | mut6 | Original HS4-650 insulator, | 81 |
| | | | with splice sites, in reverse | |
| | | | orientation | |
| pBRNGTR84 | Yes | mut6 | Original HS4-650 insulator, | 56 |
| | | | with splice sites, in reverse | |
| | | | orientation | |
| pBRNGTR87 | No | mut6 | Mutated HS4-650 insulator | 82 |
| | | | reverse orientation | |
| | | | two A→T point mutations | |
| | | | with the purpose of | |
| | | | abrogating splice acceptor | |
| | | | activity at SA1 and SA2 | |
| pBRNGTR88 | Yes | mut6 | Mutated HS4-650 insulator | 57 |
| | | | reverse orientation | |
| | | | two A→T pointmutations | |
| | | | with the purpose of | |
| | | | abrogating splice acceptor | |
| | | | activity at SA1 and SA2 | |
| pBRNGTR91 | No | mut6 | Original HS4-650 insulator, | 83 |
| | | | forward orientation | |
| | | | inversion of HS4-650 | |
| | | | insulator (i.e. now in forward | |
| | | | orientation and in the same | |
| | | | orientation as the viral polyA | |
| | | | signal) with the purpose of | |
| | | | abrogating splicing activity | |
| pBRNGTR92 | Yes | mut6 | Original HS4-650 insulator, | 58 |
| | | | forward orientation | |
| | | | inversion of HS4-650 | |
| | | | insulator (i.e. now in forward | |
| | | | orientation and in the same | |
| | | | orientation as the viral polyA | |
| | | | signal) with the purpose of | |
| | | | abrogating splicing activity | |
| pBRNGTR119 | No | mut6 | Mutated HS4-650 insulator | 84 |
| | | | reverse orientation | |
| | | | three A→T point mutations | |
| | | | with the purpose of | |
| | | | abrogating splice acceptor | |
| | | | activity at SA1, SA2 and SA3 | |
| pBRNGTR120 | No | mut6 | Mutated HS4-650 insulator | 59 |
| | | | reverse orientation | |
| | | | three A→T point mutations | |
| | | | with the purpose of | |
| | | | abrogating splice acceptor | |
| | | | activity at SA1, SA2 and SA3 | |

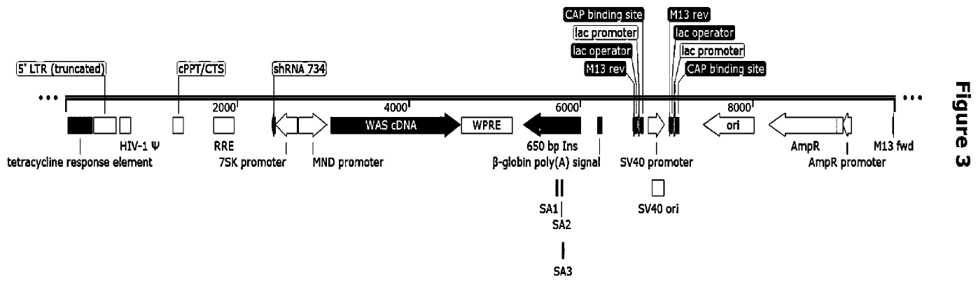

The positions and orientation of various elements within pBRNGTR84, pBRNGTR88, pBRNGTR92 and pBRNGTR120 are provided in Tables 8-11 below, and shown in FIGS. 3-6.

TABLE 8
pBRNGTR84

| | | Start (nt | End (nt |
|---|---|---|---|
| Element | Orientation | position) | position) |
| 7tetO promoter | forward | 28 | 315 |
| shRNA 734 | reverse | 2402 | 2448 |
| 7SK promoter | reverse | 2449 | 2697 |
| MND promoter | forward | 2710 | 3056 |
| WAS cDNA (wild-type ORF) | forward | 3098 | 4606 |
| WPRE | forward | 4615 | 5204 |
| cHS4 Ins-650 insulator | reverse | 5342 | 6006 |
| rabbit beta-globin | forward | 6133 | 6581 |
| polyadenylation signal | | | |

TABLE 9
pBRNGTR88

| | | Start (nt | End (nt |
|---|---|---|---|
| Element | Orientation | position) | position) |
| 7tetO promoter | forward | 28 | 315 |
| shRNA 734 | reverse | 2402 | 2448 |
| 7SK promoter | reverse | 2449 | 2697 |
| MND promoter | forward | 2710 | 3056 |
| WAS cDNA (wild-type ORF) | forward | 3098 | 4606 |
| WPRE | forward | 4615 | 5204 |
| cHS4 Ins-650 insulator | reverse | 5342 | 6006 |
| rabbit beta-globin | forward | 6133 | 6581 |
| polyadenylation signal | | | |

TABLE 10
pBRNGTR92

| | | Start (nt | End (nt |
|---|---|---|---|
| Element | Orientation | position) | position) |
| 7tetO promoter | forward | 28 | 315 |
| shRNA 734 | reverse | 2402 | 2448 |
| 7SK promoter | reverse | 2449 | 2697 |
| MND promoter | forward | 2710 | 3056 |
| WAS cDNA (wild-type ORF) | forward | 3098 | 4606 |
| WPRE | forward | 4615 | 5204 |
| cHS4 Ins-650 insulator | forward | 5345 | 6009 |
| rabbit beta-globin | forward | 6133 | 6581 |
| polyadenylation signal | | | |

TABLE 11
pBRNGTR120

| | | Start (nt | End (nt |
|---|---|---|---|
| Element | Orientation | position) | position) |
| 7tetO promoter | forward | 28 | 315 |
| shRNA 734 | reverse | 2402 | 2448 |
| 7SK promoter | reverse | 2449 | 2697 |
| MND promoter | forward | 2710 | 3056 |
| WAS cDNA (wild-type ORF) | forward | 3098 | 4606 |
| WPRE | forward | 4615 | 5204 |
| cHS4 Ins-650 insulator | reverse | 5342 | 6006 |
| rabbit beta-globin | forward | 6133 | 6581 |
| polyadenylation signal | | | |

### Example 3

Assessment of Aberrant Splicing with HMGA2 Fusion Transcript Assay

A new HDR-based gene editing assay has been developed to directly assess LV vector fusion transcripts within the HMG2A locus following integration within intron 3 of the HMGA2 gene. This approach was utilized as LV integration has been identified throughout this intron in LV trials (De Ravin et al. (2016), Science Translational Medicine, Vol. 8, pp. 335ra57).

In brief, sgRNAs targeting multiple sites within HMGA2 intron 3 were designed that exhibited high efficiency cutting in cell lines (NHEJ rates 70-90%). A series of AAV homology directed repair (HDR) donors with 0.6 kb homology arms were designed and produced. Each donor contained homology arms flanking sequences derived from the LVV LTR containing insulator elements, including modified insulators. The AAV donors were designed to be used for co-delivery with sgRNA.

Following generation of recombinant AAV vector stocks, AAV donors and sgRNAs (delivered as RNPs) were introduced into a KG-1 cell line or into primary human CD34+ cells via nucleofection. In addition to the LTR/insulator donor constructs, a control AAV HDR donor was generated containing the same homology arms designed to introduce a MND.GFP.polyadenylation cassette. This control provides a rapid means to access targeted integration rates by flow cytometry. Using this control construct, HDR rates of ˜40% were observed in KG-1 cells. Following co-delivery of RNPs and AAV LTR donors, HDR rates and fusion transcripts were measured in genomic DNA and RNA isolated from edited cells at >1 week post editing.

Preliminary experiments in KG-1 cells demonstrated efficient targeting with AAV donors with HDR rates averaging 31% (range 23-37%). Fusion transcripts utilizing the SA sites were detected in cells edited with cHS4 elements. The sequence of the fusion constructs was confirmed by DNA sequencing and matched the predicted use of the highest scoring SA sites.

Initial studies suggest modification of both SA1 and SA2 inactivates or eliminates splicing at these sites. However, fusion transcripts were observed arising from splicing at unmodified SA3 in these constructs. Fusion transcripts were not detected with constructs, which include an inverted HS4-650 insulator, i.e. in forward orientation relative to the WAS transgene, suggesting inverting the orientation eliminates the splice sites. Constructs including a combination of mutations at SA1, SA2 and SA3 were evaluated.

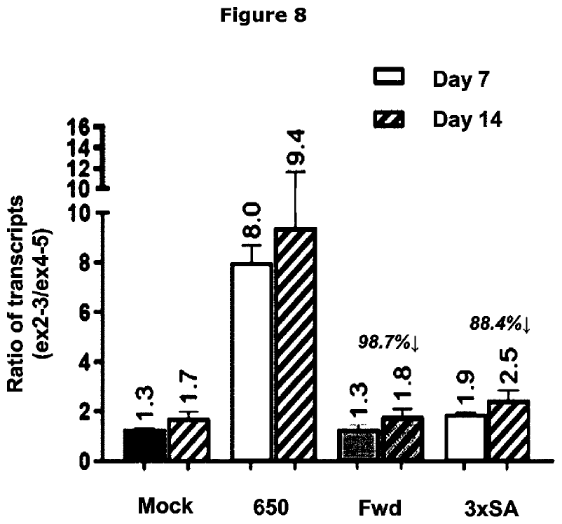

Assessment of the ratio of transcripts of HMGA2 exons 2-3/exons 4-5 by ddPCR indicated a reduction in fusion transcripts from aberrant splicing in constructs comprising a modified insulator (“3×SA” and “fwd”) when compared with constructs comprising an unmodified 650 bp insulator with unmodified splice sites (“650”); refer to FIG. 8.

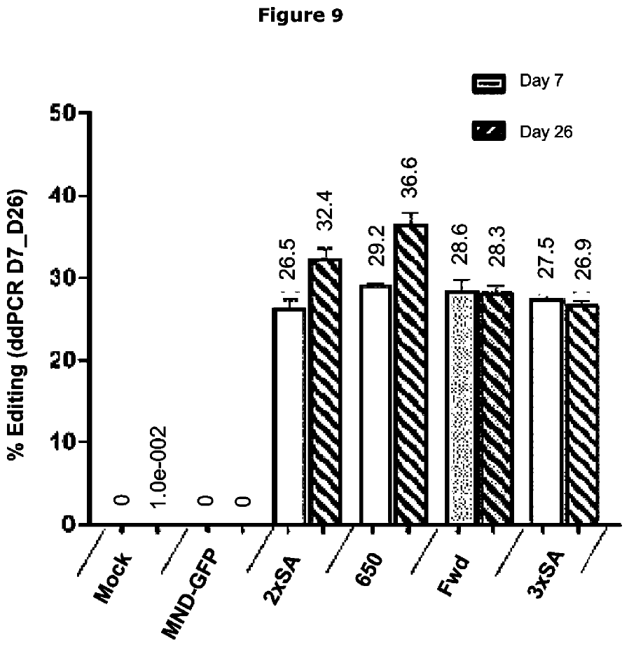

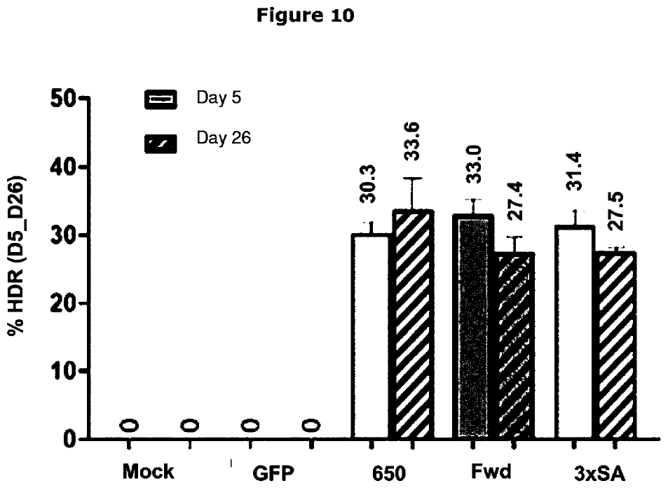

Furthermore, assessment of edited cells frequency in culture over time indicated that a reduction in HMGA2 fusion transcripts (i.e. in constructs comprising a modified insulators “fwd” and “3×SA”) correlated with a reduced or eliminated selective cell growth advantage compared with advantage observed for constructs comprising an unmodified 650 bp insulator with unmodified splice sites (“650”). Results from KG1 cells are shown in FIG. 9 and results from CD34+ cells are shown in FIG. 10.

### Example 4

RNA-Seq Methods with Enrichment for Assessment of Aberrant Splicing

Sequencing of total RNA in a sample (RNA-Seq) is a useful next-generation sequencing technique for directly assessing signals of gene expression in a sample. However, RNA-Seq samples are often highly complex, and typically require deep sequencing to fully resolve the signal of relatively rare transcripts of interest. RNA-Seq hybridization capture kits may be used to enrich targets from a complex sample prior to RNA-Seq. To this end, custom RNA baits were designed targeting HMGA2 to enrich for HMGA2 mRNA transcripts.

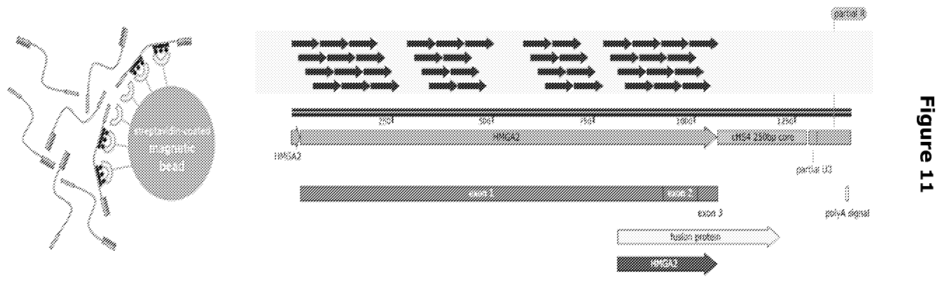

It is recognized that HMGA2 has five known transcript variants, each leading to expression of a different protein isoform. Common to all isoforms are exons 1, 2 and 3. An HMGA2 target enrichment kit was designed with baits targeting HMGA2 exons 1, 2 and 3. Baits for three housekeeping genes (B2M, PPIA, GAPDH) were also designed as controls for normalization (FIG. 11). The following protocol was used to enrich mRNAs containing HMGA2 exons 1-3 from complex RNA Seq samples, enabling aberrant splice events to be assessed through the sequencing and quantification of the abundance of downstream HMGA2 exons compared with downstream lentiviral sequence.
a) HYBRIDIZATION—Initially, a barcoded NGS cDNA library was denatured via heat, and allowed to hybridize to a complex mixture of complementary biotinylated RNA baits over the course of several hours. Adapter-specific blocking oligos were used to prevent random annealing of library molecules at the common adapter sites.
b) WASHING—After the hybridization was complete, the biotin present on each bait was bound to a streptavidin-coated magnetic bead. Wash steps assist in removal of off-target or poorly-hybridized library molecules.
c) AMPLIFICATION—The remaining library molecules bound to their complementary baits were denatured via heat, and amplified using universal library primers. This “enriched” library was sequenced and assessed.

Samples from KG1 cells (35 days post editing with AAV donors and RNPs) and CD34+ cells (13 days post editing with AAV donors and RNPs) were each assessed. All reads mapping to human genome or any of the AAV constructs were extracted. Reads mapping to i) PPIA, ii) GAPDH, iii) B2M, iv) HMGA2 or AAV (combined) were counted. Reads mapping to HMGA2 or AAV were further divided into reads mapping to: i) HMGA2 upstream of AAV insertion site; ii) HMGA2 downstream of AAV insertion site; and iii) AAV sequence.

The ratio between AAV reads and HMGA2 downstream reads can be used as a measure of splicing activity to LVV including modified insulator sequences. Expression level of AAV fusion transcripts was calculated and normalized to selected housekeeping genes (controls) described above. HMGA2 upstream reads were calulated and normalized to housekeeping genes to assess total HMGA2-expressing transcripts.

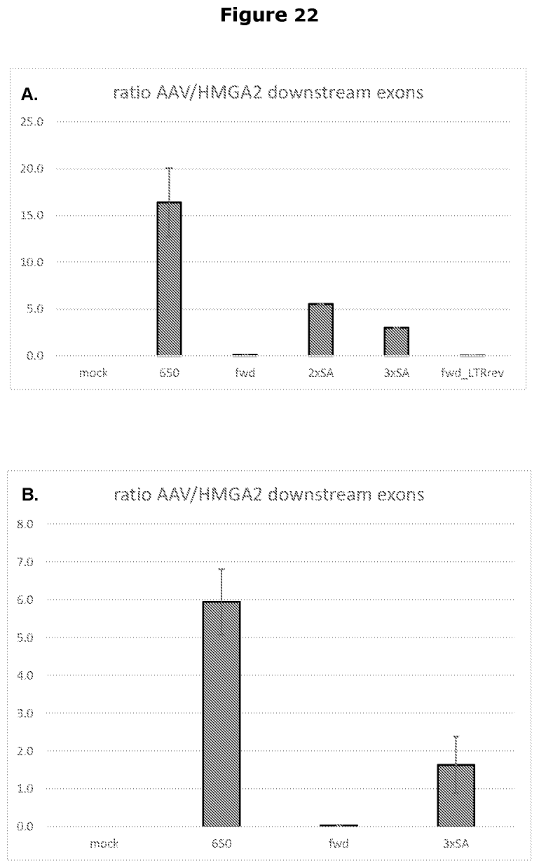

In KG1 cells, a 5.5 fold decrease in ratio of AAV/HMGA2 was observed in a construct including a 3×SA 650 bp corrected insulator, calculated as ratio between AAV reads and HMGA2 downstream exon reads (FIG. 22A). In CD34+ cells, a 3.6 fold decrease in ratio of AAV/HMGA2 was observed in a construct including a 3×SA 650 bp corrected insulator (FIG. 22B). Similar decreases in ratio of AAV/HMGA2 were observed in a construct comprising a HS4-650 insulator in a forward orientation relative to transgene (reverse orientation relative to control construct comprising original unmodified insulator (FIGS. 22A and 22B; “fwd”).

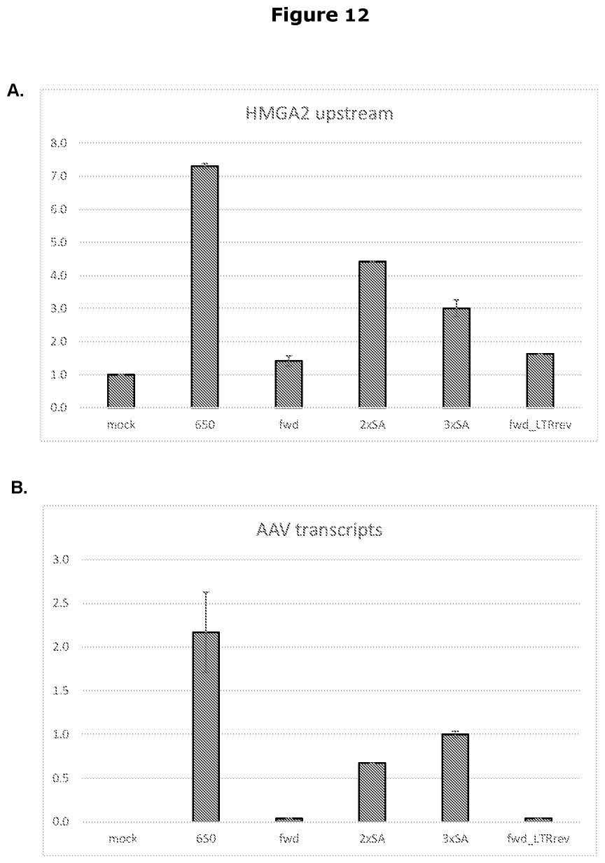

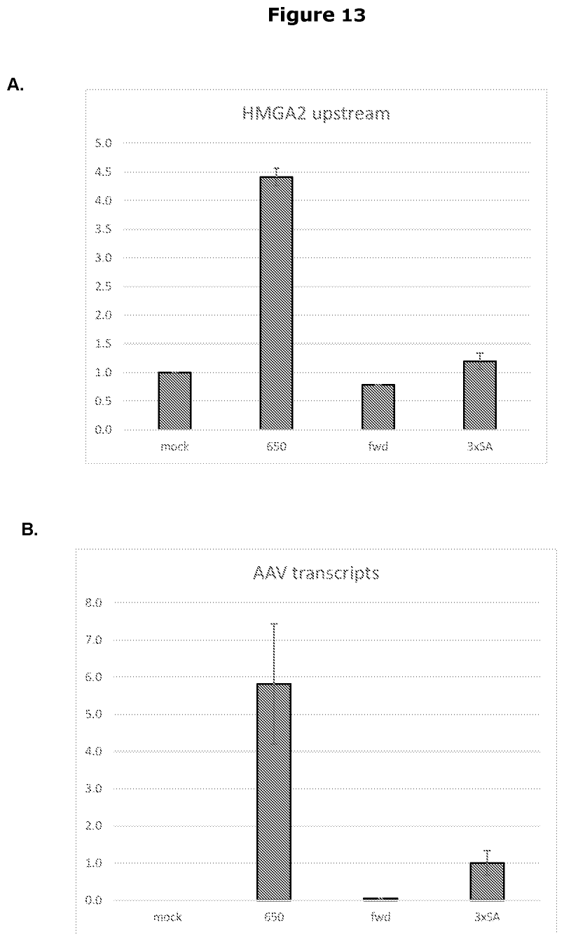

FIG. 12 and FIG. 13 show the expression level of HMGA2 transcripts and AAV fusion transcripts in KG1 and CD34+ cells, respectively. Specifically, the FIGS. 12A and 13A shows the level of total HMGA2-expressing transcripts compared to untreated cells. FIGS. 12B and 13B show the level of fusion transcripts expressed in cells normalized to the 3×SA modified insulator construct. “650” refers to construct comprising unmodified HS4-650 insulator with unmodified splice sites; “2×SA” refers to construct comprising a HS4-650 insulator with two corrected cryptic splice acceptor sites; “3×SA” refers to construct comprising a HS4-650 insulator with three corrected cryptic splice acceptor sites; “fwd” refers to a construct comprising a HS4-650 insulator in a forward orientation relative to transgene (or a reverse orientation relative to control construct comprising original unmodified insulator); and “mock” or “fwd_LTRrev” refer to controls. In both KG1 cells and CD34+ cells, constructs comprising a modified insulators (“3×SA” and “fwd”) exhibited reduced expression level of AAV fusion transcripts compared with constructs comprising an unmodified insulator (“650”).

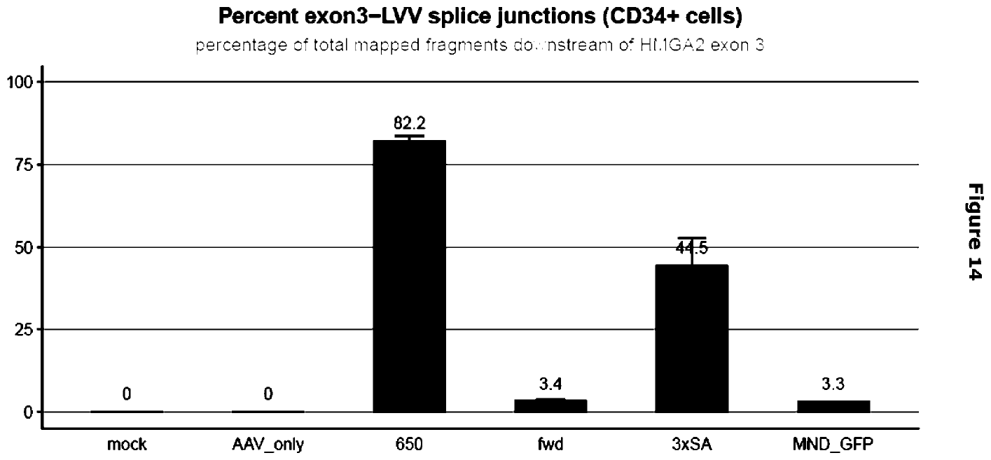

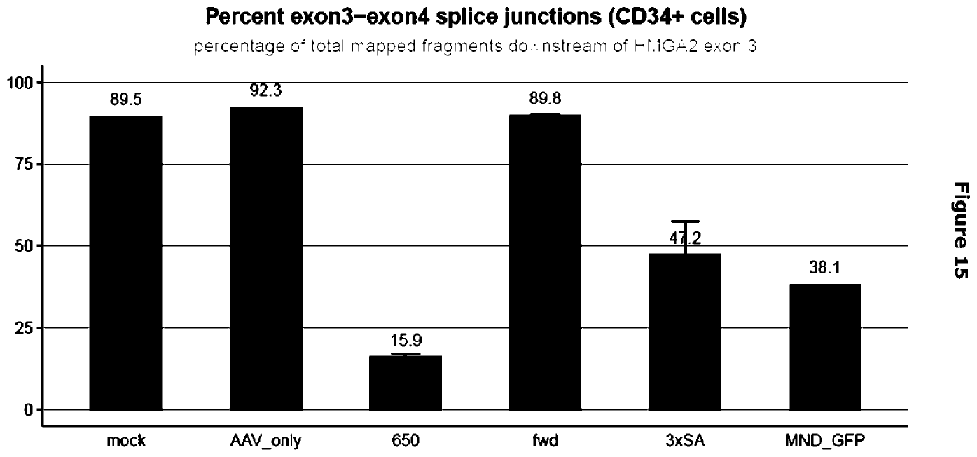

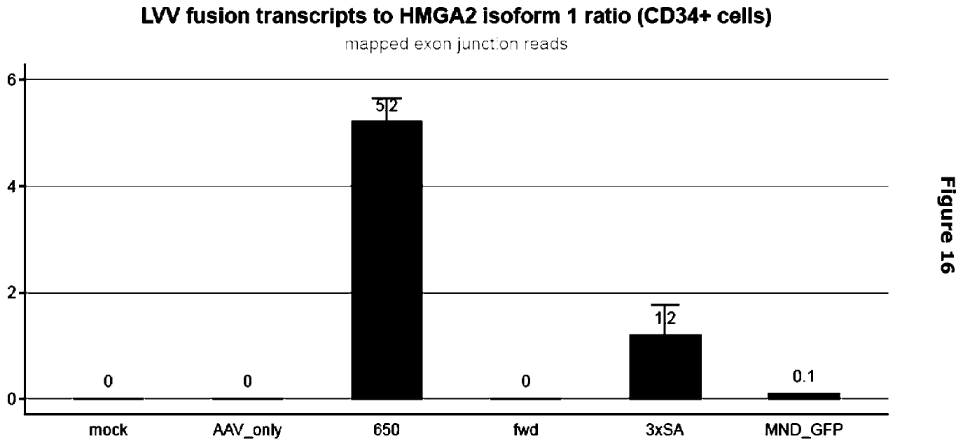

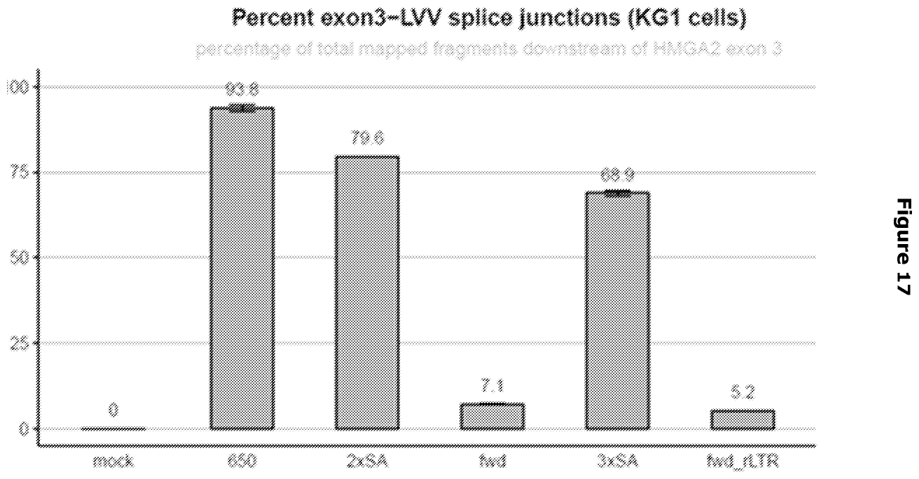

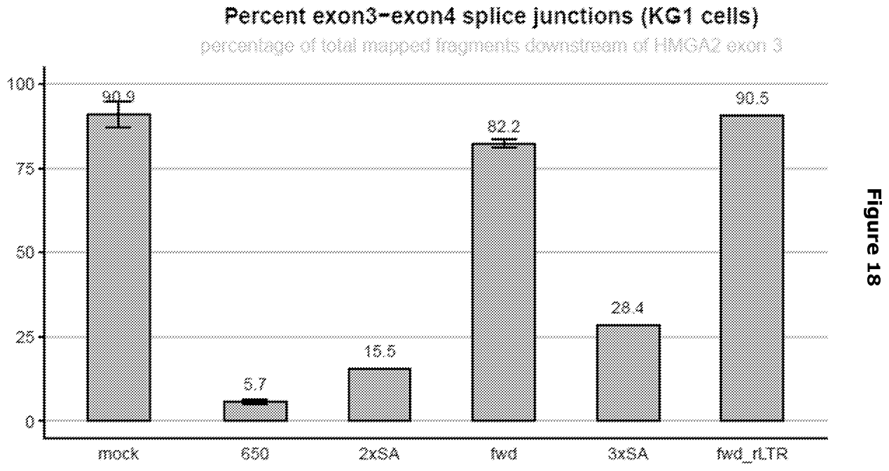

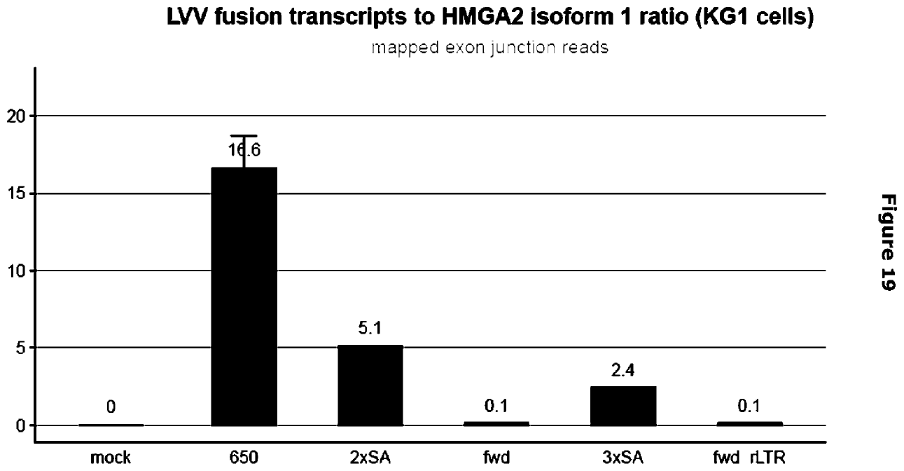

In order to quantify the detected splicing events between HMGA2 exon 3 and either exon 4 (major isoform) or the LVV sequence (AAV insert), reads containing HMGA2 exon 3 sequence were extracted and the junctions to the next exon downstream were quantified. This analysis was performed for both CD34+(FIGS. 14 to 16) and KG1 cells (FIGS. 17 to 19). In both cell types, the percentage of HMGA2 exon3-LVV splicing was decreased in both constructs comprising a modified insulator compared to constructs comprising an unmodified HS4-650 insulator with unmodified splice sites (FIGS. 14 and 17).

### Example 5

### Assessment of Insulator Activity

To assess the enhancer blocking activity of the modified HS4-650 insulator, a LIM domain only two (LMO2) activation assay may be used to verify the function of modified insulators. Similar assays have been described in Ryu et al., 2008, Blood, Vol. 111, pp. 1866 and Goodman et al. 2018, Journal of Virology, Vol. 92 pp. e01639-17.

In brief, Jurkat cell lines having a targeted integration of a provirus within the promoter or the first intron of the LMO2 gene are used to assess vector constructs (Ryu et al., 2008; Zhou et al., 2010). Where a modified insulator sequence retains its insulator enhancer-blocking function, a LMO2 expression similar to that of the unmodified insulator sequence is observed, corresponding to a clear reduction in LMO2 expression compared to an uninsulated provirus. Where insulator function is reduced or disrupted by modification of the insulator sequence, a LMO2 expression higher than that of the unmodified insulator sequence is observed.

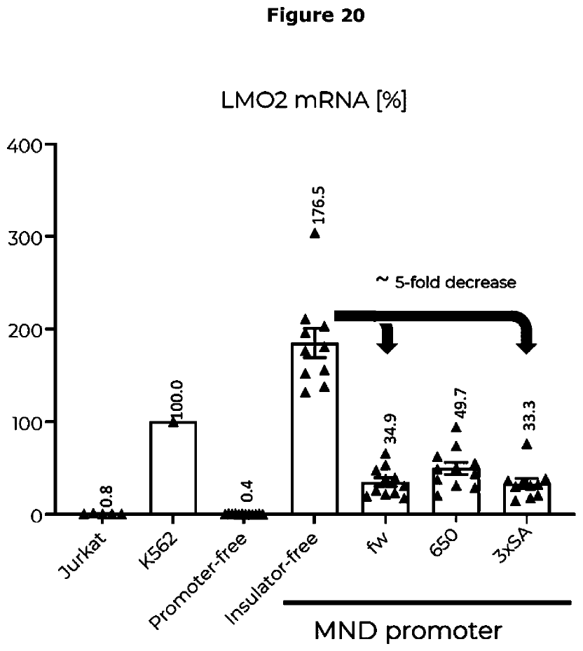

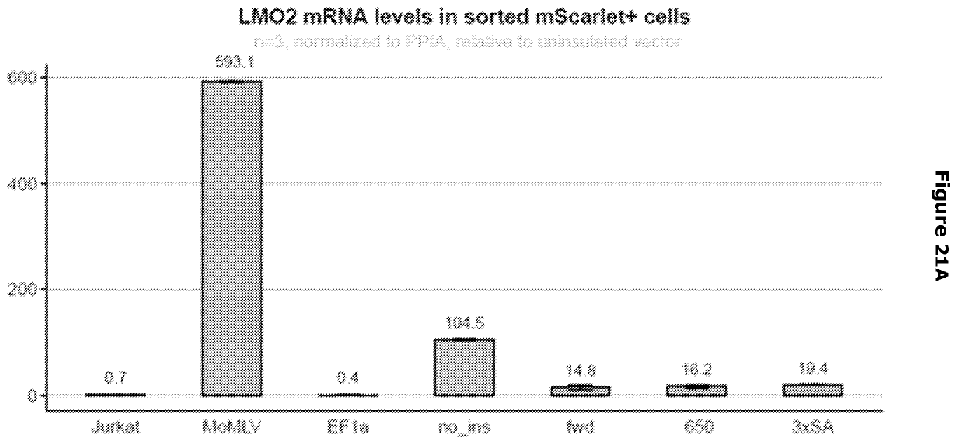

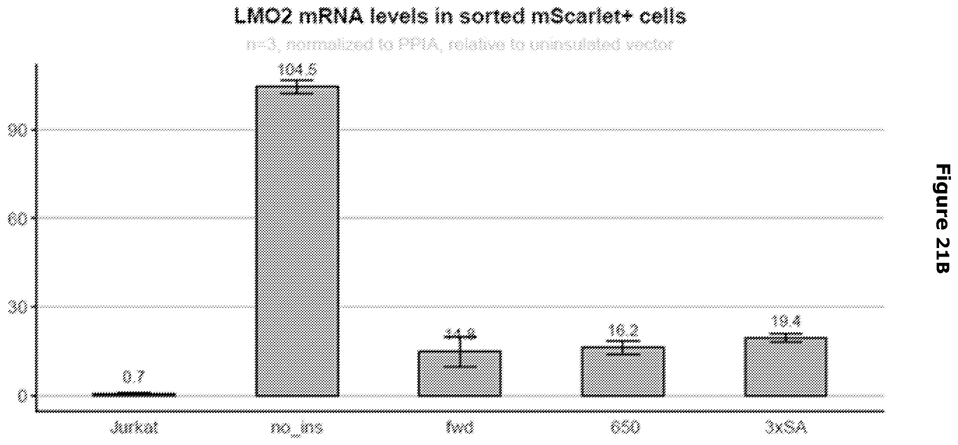

RT-qPCR was used as a measurement for LMO2 expression and thus enhancer blocking activity of exemplary insulators. LVV provirus constructs with an MND promoter driving an mScarlet-I reporter transgene and harboring different insulator sequences in the LTRs were used for this assay. A MoMLV provirus and a LVV provirus construct with an MND promoter driving an mScarlet-I reporter transgene but lacking an insulator were used as positive controls for LMO2 activation. A provirus construct without promoter or with an EFlalpha promoter were used as a negative control for LMO2 activation. Both single clone assay (FIG. 20) and bulk cell assays (FIGS. 21A and 21B) were completed. Exemplary constructs comprising a modified insulator (“fwd” and “3×SA”) demonstrated comparable LMO2 expression relative to the unmodified HS4-650 insulator with unmodified splice sites (“650”) indicating the modified insulators retain the desired insulator enhancer-blocking function. Positive and negative controls included a construct lacking a promoter (“Promoter-free”) and a construct lacking an insulator (“Insulator-free” or “no-Ins”).

### Example 6

### In Vitro Immortalization Assay (IVIM Assay)

An in vitro immortalization assay (IVIM assay) is used to assess vector-mediated genotoxic events after gene therapy with the lentiviral vectors. IVIM is a rapid mutagenesis assay using a simple cell culture model to quantify the risk of hematopoietic cell transformation. IVIM assay may be able to quantify the incidence of genetoxic mutants based on the initial number of transduced cells and the clonal characterization of the mutants that show robust replating after limiting dilution. It also enables characterisation of transforming common insertion sites (CIS).

The IVIM assay has been described (Modlich et al. (2006), Blood, Vol. 108 pp. 2545; and Modlich et al. (2009), Molecular Therapy, Vol. 17 pp. 1919-1928). Briefly, Lineage-negative (Lin−) bone marrow cells are isolated from complete bone marrow. Pre-stimulated Lin− bone marrow cells are transduced with LVV vector by Spinoculation (retronectin coated suspension culture dishes). After two rounds of transductions, and cells are harvested for flow cytometry (FACS) and DNA samples for real-time PCR analysis (copy number).

After transduction, cells are expanded as mass cultures for approximately two weeks. After mass culture expansion, cells are plated into 96-well plates, Approximately two weeks later, positive wells are counted, and the frequency of replating cells is calculated. Selected clones may be expanded for further characterization.

### Example 7

### Transduction and WASP Expression

WAS expression from exemplary lentiviral vector constructs were assessed in two different cell types: Murine lineage negative (Lin^{neg}) cells and (human) U937 cells. WAS KO and WT cells in both the cell types were used as positive controls for the assay.

Murine Lin^{neg} cells: Lineage negative cells from bone marrow of WAS KO mice was performed by magnetic labelling using the Direct Lineage Cell Depletion Kit (Miltenyi). After Lineage depletion, 200.000 Lin^{neg} WASp KO cells per transduction condition were mixed with 150 μL medium containing transduction enhancers (TEs; 1× LentiBoost and 10 μM dmPGE2) in a 96 well and were incubated for 1 h at 37° C. and 5% CO₂. Different WASP LVs was added to the cells at MOIs of 1 and 10 to a final volume of 200 μL per well and incubated for 12-16 h at 37° C. and 5% CO₂. Each transduction was performed in triplicate wells. After the stipulated incubation time, cells were washed with medium to remove the viral supernatant and cultured for 7 days (in a 24 well plate).

U937 cells: U937 is a pro-monocytic, human myeloid leukemia cell line, known to be expressing high level of WASP. For WASP LV characterization, U937 WASP KO cells (clone 19 B) were used. WAS KO clone was generated via CRISPR/Cas9 targeting of Exon 7 of the WASP gene locus. Like murine Lin^{neg} cells, WAS KO U937 cells were transduced with various WAS LVs and incubated for 12-16 h at 37° C. and 5% CO₂. The MOIs used for the U937 cells are: 0.5, 1 and 10. As with murine Lin^{neg} cells, each transduction was performed in triplicates and cells were cultured (in 24 well plate) for 21 days post transduction.

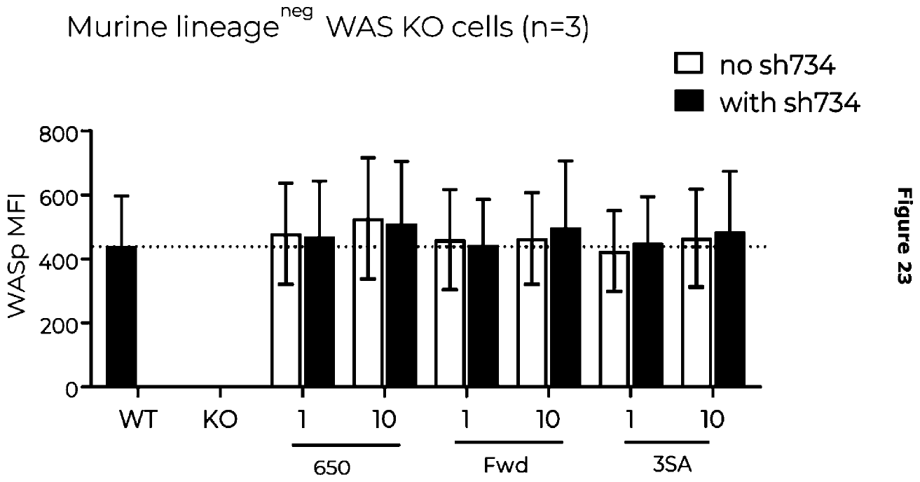

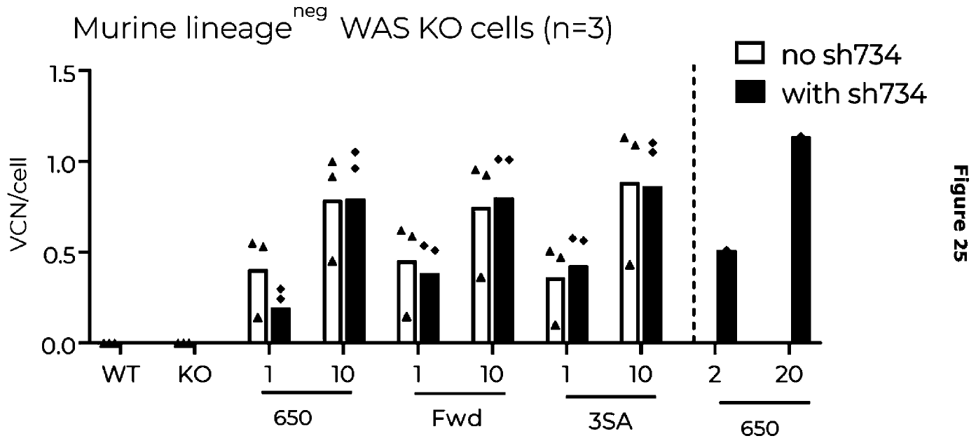

WAS protein expression was analyzed at 7 days post transduction for Lin^{neg} cells and at 21 days post transduction for U937 cells. Briefly, cells were harvested and permeabalized to allow for the staining of WAS protein intracellularly. WAS protein was stained with Alexa-Fluor 647 labelled WAS antibody (5A5, BD Biosciences, labelled in-house). Untransduced WAS KO cells and WT cells (for Lin^{neg} and U937 cells) were used as negative and positive controls respectively and WAS expression is expressed as Median Fluorescent Intensity (MFI). In Lin^{neg} cells, comparable WASP expression was observed among all LVVs, including those with modified insulators. In U937 cells, WASP expression exceeded WT controls (WT:KO 1-2 and 1-3). This indicates modification of insulators to address aberrant splicing did not reduce or hinder transgene expression (see FIG. 23 for Lin^{neg} cells and FIG. 25 for U937 cells).

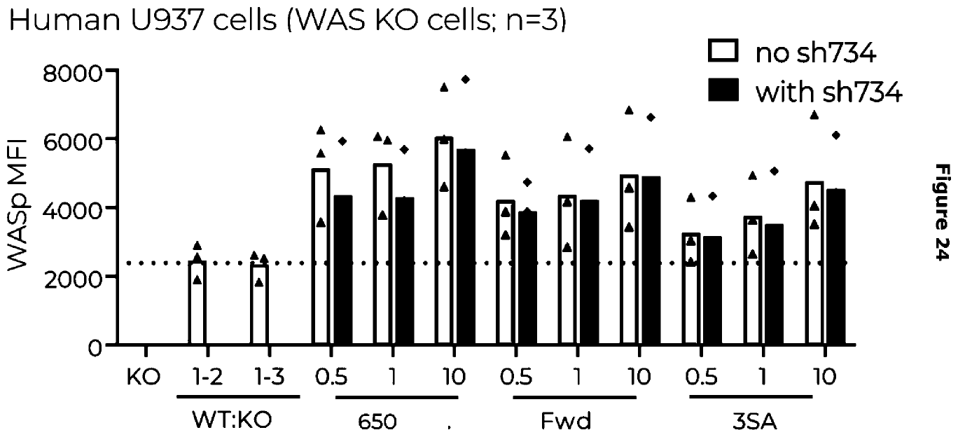

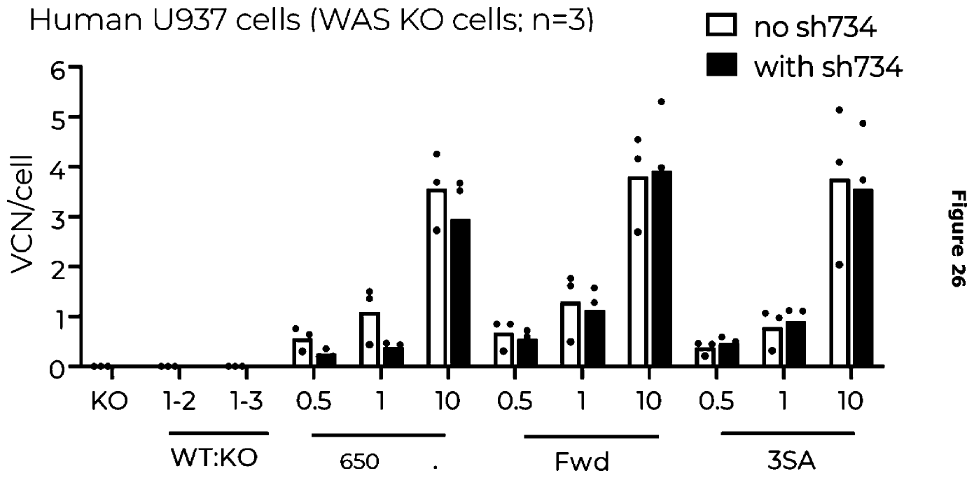

In addition to the WAS protein expression, total DNA from the cells was extracted and the number of vector copy integrations (VCN) were analyzed by q-PCR and is expressed as VCN/cell. In both the cell types dose dependent increase in VCN was observed (see FIG. 24 for Lin^{neg} cells and FIG. 26 for U937 cells).

### Example 8

Transplantation of was KO Mice with LVV Modified Cells

In vivo WAS rescue is assessed in a WAS KO mouse model. To this extent, WAS KO murine Lin^{neg} cells are modified with the same protocol described in Example 6 with exemplary WAS LVVs with corrected insulators. After LV modifications, cells are washed and transplanted into pre-conditioned (lethal irradiation) WAS KO mice (˜2×10⁶ cells/mouse±20%). The cells from donor mice and the recipient mice are distinguishable based on the CD45.1 or CD45.2 congeneic alleles. WT to WAS KO and WAS KO to WAS KO groups are used as positive and negative controls, respectively. Peripheral blood from the mice is drawn at regular intervals to monitor the engraftment and development of various immune cell lineages and the WASP expression in the respective cell types. In order to assess the function of WASP rescue, T cells from the spleens of transplanted mice (upon sacrifice) are analyzed for their function in response to stimulus.

### Example 9

### Identification of Cryptic Splice Sites in SCD LVV

A lentiviral vector containing a human γ-globin transgene was assessed for cryptic splice sites. This lentiviral vector is the plasmid pCalH10_TL20c_rGbGM_7SKsh734 (“pCalH10”) having a sequence set forth in SEQ ID NO:109. As can be seen from FIG. 27, pCalH10 contains a human γ-globin^{G16D} expression cassette that contains the human γ-globin (HBG) exons (with the G16D point mutation), the β-globin (HBB) non-coding sequences, a β-globin promoter, and a 3.2 kb β-globin locus control region (LCR) consisting of hypersensitivity sites (HS2, HS3, and HS4 elements), cloned in reverse orientation to the viral RNA transcripts in the viral backbone. The β-globin non-coding region includes a truncated HBB intron 2. A second expression cassette, which is downstream of the human γ-globin^{G16D} expression cassette and in the forward orientation, includes a 7sk promoter operably linked to nucleic acid encoding shRNA 734 (sh734). Downstream of this in the LTR is a HS4-400 insulator in the reverse orientation. Transcription of lentiviral DNA is driven by the 7tetO promoter/operator (see FIG. 27).

Bioinformatic splice site prediction analysis (Netgene2) was used to identify potential splice sites in pCalH10. This revealed approximately one hundred potential splice sites, suggesting that there may be aberrant splicing associated with this vector.

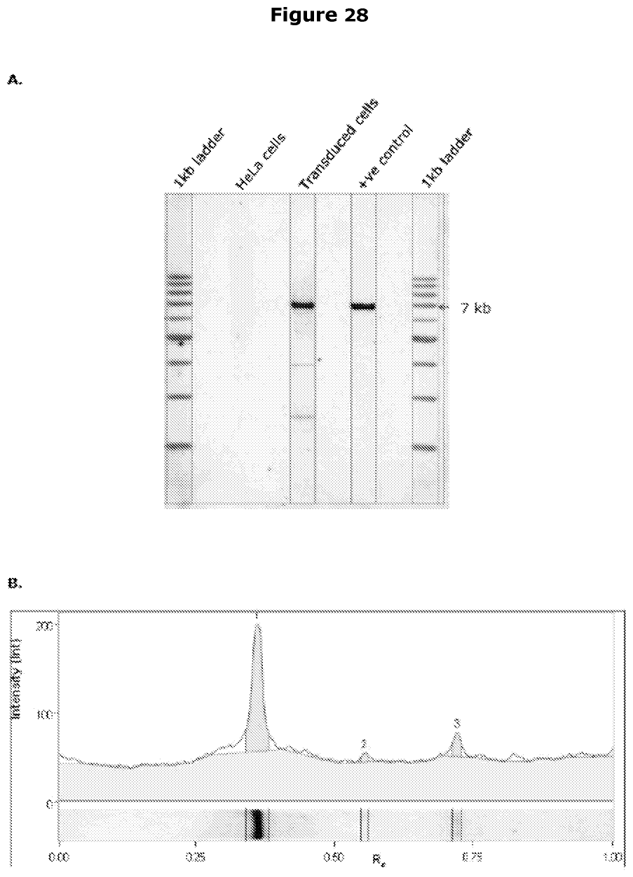

The stability of the pCalH10 vector insert was then analysed. Briefly, HeLa cells were transduced with virions produced from pCalH10 and therefore containing the same insert described above and shown in FIG. 27. Cellular DNA was extracted 6 days later and the DNA was subjected to restriction enzyme digest and Southern blot analysis, and compared to non-transduced HeLa cells (negative control) and pCalH10 (positive control). AflIII and NotI restriction enzymes were used to on the test sample to release the vector insert sequence of approximately 6.7 kb, and MfeI and XbaI were used to generate probe fragments from pCalH10. As shown in FIG. 28, a fragment of approximately 7 kb was observed as being the dominant species (87.5%), with fragments of approximately 4 kb and 2.5 kb as being minor species (3% and 9.5%, respectively).

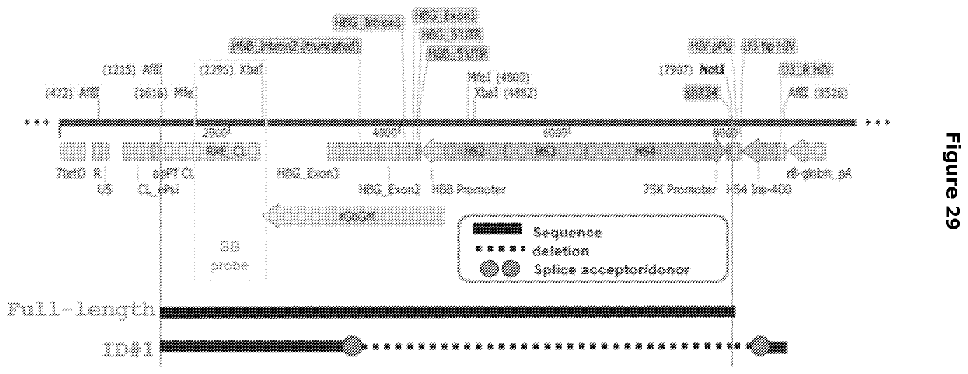

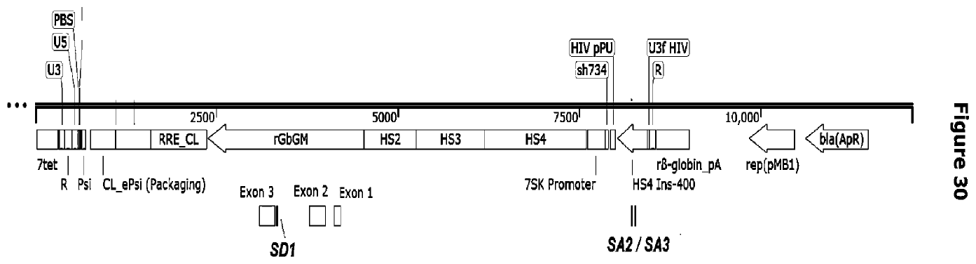

Next Generation Sequencing of the DNA obtained from transduced cells was also performed, to identify the donor and acceptor sites that contributed to the generation of the truncated 2.5 kb fragment that constituted 9.5% of the population. It was determined that the splice donor site is a splice donor site (SD1) in the positive strand of the vector in the truncated β-globin intron 2 within the γ-globin transgene. As the γ-globin transgene is present in the vector in the reverse orientation, SD1 is in the complementary strand of the γ-globin transgene, i.e. in the reverse, complement sequence of the γ-globin transgene. The splice acceptor site (SA2) is on the positive strand of the vector in the HS4-400 insulator. As the HS4-400 insulator is in the reverse orientation in pCalH10, SA2 is on the complementary strand (i.e. in the reverse complement sequence) of the HS4-400 insulator. Aberrant splicing at these sites results in a truncated lentiviral fragment in which part of the γ-globin expression construct, the β-globin LCR and the 7sk-sh734 expression construct are deleted (FIG. 29). Table 12 below sets forth the details of SD1 and SA2, and a further splice acceptor site, SA3 (see FIG. 30) that was deemed to be of concern.

TABLE 12

| Splice | pos | | | Sequence of splice |
|---|---|---|---|---|
| | 5′->3′ | | Confidence | site in vector |
| site | in vector | strand | score | 5′ intron^{∧}exon 3′ |
| SD1 | 3310 | + | 0.93 | AAGATAAGAG^{∧}GTATGAACAT |
| | | | | (SEQ ID NO: 96) |
| SA2 | 8209 | + | 0.85 | ATCCCCCCAG^{∧}GTGTCTGCAG |
| | | | | (SEQ ID NO:61) |
| SA3 | 8219 | + | 0.33 | GTGTCTGCAG^{∧}GCTCAAAGAG |
| | | | | (SEQ ID NO: 62) |

Position: Nucleotide position number is of the first nucleotide in the splice site sequence as it relates to SEQ ID NO: 107
Strand: The strand in which is located the splice site; (+) = forward (−) = reverse
Confidence Score: Confidence value provided by NetGene software that estimates the probability that a given sequence is a true splice site (1 = maximum value; for splice donors (SD) a score >0.5 is considered significant; for splice acceptors (SA) a score of >0.2 is considered significant).
^{∧}denotes splice site

### Example 10

Generation of Vectors with Inactivated Splice Sites

A series of modified vectors was generated to inactivate SD1, SA2 and/or SA3. Most of these vectors were based on pCalH10 and contain a mutation in the sequence of one or more of the splice sites so as to inactivate them. For those vectors that contained a mutation in the γ-globin transgene to inactivate SD1, a G to A mutation was made, and for those vectors that contained a mutation in the HS4-400 insulator to inactivate SA2 and/or SA3, the mutation was an A to T mutation, as shown below in Table 13. In two vectors, the HS4-400 insulator was simply deleted. Table 14 summarizes the vectors produced.

TABLE 13

| | Sequence of splice site | Sequence of inactivated | |
|---|---|---|---|
| Splice | in pTL20c vector | splice site in modified | Modified vectors |
| site | 5′ intron^{∧}exon 3′ | vector | with inactivation |
| SD1 | AAGATAAGAG^{∧}GTATGAACAT | AAGATAAGAG^{∧}ATATGAACAT | pCalH10 |
| | (SEQ ID NO: 96) | (SEQ ID NO: 99) | pCalH13 |
| | | | pCalH11 |
| | | | pCalH12 |
| SA2 | ATCCCCCCAG^{∧}GTGTCTGCAG | ATCCCCCCTG^{∧}GTGTCTGCAG | pCalH32 |
| | (SEQ ID NO: 61) | (SEQ ID NO: 64) | pCalH13 |
| | | | pCalH31 |
| | | | pCalH12 |
| SA3 | GTGTCTGCAG^{∧}GCTCAAAGAG | GTGTCTGCTG^{∧}GCTCAAAGAG | pCalH32 |
| | (SEQ ID NO: 62) | (SEQ ID NO: 65) | pCalH13 |

TABLE 14

| | | | SEQ |
|---|---|---|---|
| | | | ID |
| Vector | Globin | Insulator | NO: |
| pCalH10 | Original β/γ-globin | Original HS4-400 insulator, | 107 |
| | transgene | with splice sites, in reverse | |
| | | orientation | |
| pCalH20 | Original β/γ -globin | No HS4-400 insulator | 108 |
| | transgene | | |
| pCalH21 | G → A point mutation in | No HS4-400 insulator | 109 |
| | intron 2 of β/γ -globin | | |
| | transgene with the | | |
| | purpose of abrogating | | |
| | splice donor activity | | |
| | at SD1 | | |
| pCalH32 | Original β/γ -globin | Mutated HS4-400 insulator | 110 |
| | transgene | reverse orientation | |
| | | two A→T point mutations | |
| | | with the purpose of | |
| | | abrogating splice acceptor | |
| | | activity at SA2 and SA3 | |
| pCalH13 | G → A point mutation in | Mutated HS4-400 insulator | 111 |
| | intron 2 of β/γ -globin | reverse orientation | |
| | transgene with the | two A→T point mutations | |
| | purpose of abrogating | with the purpose of | |
| | splice donor activity | abrogating splice acceptor | |
| | at SD1 | activity at SA2 and SA3 | |
| pCalH11 | G → A point mutation in | Original HS4-400 insulator, | 112 |
| | intron 2 of β/γ -globin | reverse orientation | |
| | transgene with the | | |
| | purpose of abrogating | | |
| | splice donor activity | | |
| | at SD1 | | |
| pCalH31 | Original b/g-globin | Mutated HS4-400 insulator | 113 |
| | transgene | reverse orientation | |
| | | one A→T point mutation | |
| | | with the purpose of | |
| | | abrogating splice acceptor | |
| | | activity at SA2 | |
| pCalH12 | G → A point mutation in | Mutated HS4-400 insulator | 114 |
| | intron 2 of β/γ -globin | reverse orientation | |
| | transgene with the | one A→T point mutation | |
| | purpose of abrogating | with the purpose of | |
| | splice donor activity | abrogating splice acceptor | |
| | at SD1 | activity at SA2 | |

### Example 11

### Assessment of Insulator Activity

To assess the activity of the modified HS4-400 insulator, a LIM domain only two (LMO2) activation assay may be used to verify the function of modified insulators. Similar assays have been described in Ryu et al. (2008), Blood, Vol. 111, pp. 1866 and Goodman et al. (2018), Journal of Virology, Vol. 92 pp. e01639-17.

In brief, Jurkat cell lines having a targeted integration site within the promoter or the first intron of the LMO2 gene are used to assess vector constructs (Ryu et al. (2008); Zhou et al. (2010), Blood, Vol. 116(6), pp. 900-908). Where a modified insulator sequence retains its insulator function, a reduction in LMO2 expression is observed. Where insulator function is reduced or disrupted by modification of the insulator sequence, little or no reduction of LMO2 expression is observed.

### Example 12

### In Vitro Immortalization Assay (IVIM Assay)

An in vitro immortalization assay (IVIM assay) can be used to assess vector-mediated genotoxic events after gene therapy with the lentiviral vectors. IVIM is a rapid mutagenesis assay using a simple cell culture model to quantify the risk of hematopoietic cell transformation. IVIM assay may be able to quantify the incidence of genetoxic mutants based on the initial number of transduced cells and the clonal characterization of the mutants that show robust replating after limiting dilution. It also enables characterisation of transforming common insertion sites (CIS).

The IVIM assay has been described (Modlich et al. (2006), Blood, Vol. 108 pp. 2545; and Modlich et al. (2009), Molecular Therapy, Vol. 17 pp. 1919-1928). Briefly, Lineage-negative (Lin−) bone marrow cells are isolated from complete bone marrow. Pre-stimulated Lin− bone marrow cells are transduced with LVV vector by Spinoculation (retronectin coated suspension culture dishes). After two rounds of transductions, and cells are harvested for flow cytometry (FACS) and DNA samples for real-time PCR analysis (copy number).

After transduction, cells are expanded as mass cultures for approx. two weeks. After mass culture expansion, cells are plated into 96-well plates, Approx. two weeks later, positive wells are counted, and the frequency of replating cells is calculated. Selected clones may be expanded for further characterization.

Sequences

Unmodified HS4-650 insulator (SEQ ID NO: 1)
GAGCTCACGGGGACAGCCCCCCCCCAAAGCCCCCAGGGATGTAATTACGTCCCTCCCCCGCTAGGG
GGCAGCAGCGAGCCGCCCGGGGCTCCGCTCCGGTCCGGCGCTCCCCCCGCATCCCCGAGCCGGCA
GCGTGCGGGGACAGCCCGGGCACGGGGAAGGTGGCACGGGATCGCTTTCCTCTGAACGCTTCTCGC
TGCTCTTTGAGCCTGCAGACACCTGGGGGGATACGGGGAAAAAGCTTGATATCATGTGTCTGAGCCT
GCATGTTTGATGGTGTCTGGATGCAAGCAGAAGGGGTGGAAGAGCTTGCCTGGAGAGATACAGCTG
GGTCAGTAGGACTGGGACAGGCAGCTGGAGAATTGCCATGTAGATGTTCATACAATCGTCAAATCAT
GAAGGCTGGAAAAGCCCTCCAAGATCCCCAAGACCAACCCCAACCCACCCACCGTGCCCACTGGCCA
TGTCCCTCAGTGCCACATCCCCACAGTTCTTCATCACCTCCAGGGACGGTGACCCCCCCACCTCCGTG
GGCAGCTGTGCCACTGCAGCACCGCTCTTTGGAGAAGGTAAATCTTGCTAAATCCAGCCCGACCCTC
CCCTGGCACAACGTAAGGCCATTATCTCTCATCCAACTCCAGGACGGAGTCAGTGAGGATGGGGCT
Reverse complement (r-c) of unmodified HS4-650 insulator (SEQ ID NO: 2)
AGCCCCATCCTCACTGACTCCGTCCTGGAGTTGGATGAGAGATAATGGCCTTACGTTGTGCCAGGGG
AGGGTCGGGCTGGATTTAGCAAGATTTACCTTCTCCAAAGAGCGGTGCTGCAGTGGCACAGCTGCCC
ACGGAGGTGGGGGGGTCACCGTCCCTGGAGGTGATGAAGAACTGTGGGGATGTGGCACTGAGGGA
CATGGCCAGTGGGCACGGTGGGTGGGTTGGGGTTGGTCTTGGGGATCTTGGAGGGCTTTTCCAGCC
TTCATGATTTGACGATTGTATGAACATCTACATGGCAATTCTCCAGCTGCCTGTCCCAGTCCTACTGAC
CCAGCTGTATCTCTCCAGGCAAGCTCTTCCACCCCTTCTGCTTGCATCCAGACACCATCAAACATGCA
GGCTCAGACACATGATATCAAGCTTTTTCCCCGTATCCCCCCAGGTGTCTGCAGGCTCAAAGAGCAGC
GAGAAGCGTTCAGAGGAAAGCGATCCCGTGCCACCTTCCCCGTGCCCGGGCTGTCCCCGCACGCTG
CCGGCTCGGGGATGCGGGGGGAGCGCCGGACCGGAGCGGAGCCCCGGGCGGCTCGCTGCTGCCC
CCTAGCGGGGGAGGGACGTAATTACATCCCTGGGGGCTTTGGGGGGGGGCTGTCCCCGTGAGCTC
Modified HS4-650 insulator (r-c)-A to T mutation at SA1 (mutation in bold and underlined)
(SEQ ID NO: 3)
AGCCCCATCCTCACTGACTCCGTCCTGGAGTTGGATGAGAGATAATGGCCTTACGTTGTGCCAGGGG
AGGGTCGGGCTGGATTTAGCAAGATTTACCTTCTCCAAAGAGCGGTGCTGCAGTGGCACAGCTGCCC
ACGGAGGTGGGGGGGTCACCGTCCCTGGAGGTGATGAAGAACTGTGGGGATGTGGCACTGAGGGA
CATGGCCAGTGGGCACGGTGGGTGGGTTGGGGTTGGTCTTGGGGATCTTGGAGGGCTTTTCCAGCC
TTCATGATTTGACGATTGTATGAACATCTACATGGCAATTCTCCAGCTGCCTGTCCCAGTCCTACTGAC
CCAGCTGTATCTCTCCAGGCAAGCTCTTCCACCCCTTCTGCTTGCATCCTGACACCATCAAACATGCA
GGCTCAGACACATGATATCAAGCTTTTTCCCCGTATCCCCCCAGGTGTCTGCAGGCTCAAAGAGCAGC
GAGAAGCGTTCAGAGGAAAGCGATCCCGTGCCACCTTCCCCGTGCCCGGGCTGTCCCCGCACGCTG
CCGGCTCGGGGATGCGGGGGGAGCGCCGGACCGGAGCGGAGCCCCGGGCGGCTCGCTGCTGCCC
CCTAGCGGGGGAGGGACGTAATTACATCCCTGGGGGCTTTGGGGGGGGGCTGTCCCCGTGAGCTC
Modified HS4-650 insulator (r-c)-A to T mutations at SA1 and SA2 (mutations in bold and
underlined) (SEQ ID NO: 4)
AGCCCCATCCTCACTGACTCCGTCCTGGAGTTGGATGAGAGATAATGGCCTTACGTTGTGCCAGGGG
AGGGTCGGGCTGGATTTAGCAAGATTTACCTTCTCCAAAGAGCGGTGCTGCAGTGGCACAGCTGCCC
ACGGAGGTGGGGGGGTCACCGTCCCTGGAGGTGATGAAGAACTGTGGGGATGTGGCACTGAGGGA
CATGGCCAGTGGGCACGGTGGGTGGGTTGGGGTTGGTCTTGGGGATCTTGGAGGGCTTTTCCAGCC
TTCATGATTTGACGATTGTATGAACATCTACATGGCAATTCTCCAGCTGCCTGTCCCAGTCCTACTGAC
CCAGCTGTATCTCTCCAGGCAAGCTCTTCCACCCCTTCTGCTTGCATCCTGACACCATCAAACATGCA
GGCTCAGACACATGATATCAAGCTTTTTCCCCGTATCCCCCCTGGTGTCTGCAGGCTCAAAGAGCAGC
GAGAAGCGTTCAGAGGAAAGCGATCCCGTGCCACCTTCCCCGTGCCCGGGCTGTCCCCGCACGCTG
CCGGCTCGGGGATGCGGGGGGAGCGCCGGACCGGAGCGGAGCCCCGGGCGGCTCGCTGCTGCCC
CCTAGCGGGGGAGGGACGTAATTACATCCCTGGGGGCTTTGGGGGGGGGCTGTCCCCGTGAGCTC
Modified HS4-650 insulator (r-c)-A to T mutations at SA1, and SA3 (mutations in bold and
underlined) (SEQ ID NO: 5)
AGCCCCATCCTCACTGACTCCGTCCTGGAGTTGGATGAGAGATAATGGCCTTACGTTGTGCCAGGGG
AGGGTCGGGCTGGATTTAGCAAGATTTACCTTCTCCAAAGAGCGGTGCTGCAGTGGCACAGCTGCCC
ACGGAGGTGGGGGGGTCACCGTCCCTGGAGGTGATGAAGAACTGTGGGGATGTGGCACTGAGGGA
CATGGCCAGTGGGCACGGTGGGTGGGTTGGGGTTGGTCTTGGGGATCTTGGAGGGCTTTTCCAGCC
TTCATGATTTGACGATTGTATGAACATCTACATGGCAATTCTCCAGCTGCCTGTCCCAGTCCTACTGAC
CCAGCTGTATCTCTCCAGGCAAGCTCTTCCACCCCTTCTGCTTGCATCCTGACACCATCAAACATGCA
GGCTCAGACACATGATATCAAGCTTTTTCCCCGTATCCCCCCAGGTGTCTGCTGGCTCAAAGAGCAGC
GAGAAGCGTTCAGAGGAAAGCGATCCCGTGCCACCTTCCCCGTGCCCGGGCTGTCCCCGCACGCTG
CCGGCTCGGGGATGCGGGGGGAGCGCCGGACCGGAGCGGAGCCCCGGGCGGCTCGCTGCTGCCC
CCTAGCGGGGGAGGGACGTAATTACATCCCTGGGGGCTTTGGGGGGGGGCTGTCCCCGTGAGCTC
Modified HS4-650 insulator (r-c)-A to T mutations at SA1, SA2 and SA3 (mutations in bold
and underlined) (SEQ ID NO: 6)
AGCCCCATCCTCACTGACTCCGTCCTGGAGTTGGATGAGAGATAATGGCCTTACGTTGTGCCAGGGG
AGGGTCGGGCTGGATTTAGCAAGATTTACCTTCTCCAAAGAGCGGTGCTGCAGTGGCACAGCTGCCC
ACGGAGGTGGGGGGGTCACCGTCCCTGGAGGTGATGAAGAACTGTGGGGATGTGGCACTGAGGGA
CATGGCCAGTGGGCACGGTGGGTGGGTTGGGGTTGGTCTTGGGGATCTTGGAGGGCTTTTCCAGCC
TTCATGATTTGACGATTGTATGAACATCTACATGGCAATTCTCCAGCTGCCTGTCCCAGTCCTACTGAC
CCAGCTGTATCTCTCCAGGCAAGCTCTTCCACCCCTTCTGCTTGCATCCTGACACCATCAAACATGCA
GGCTCAGACACATGATATCAAGCTTTTTCCCCGTATCCCCCCTGGTGTCTGCTGGCTCAAAGAGCAGC
GAGAAGCGTTCAGAGGAAAGCGATCCCGTGCCACCTTCCCCGTGCCCGGGCTGTCCCCGCACGCTG
CCGGCTCGGGGATGCGGGGGGAGCGCCGGACCGGAGCGGAGCCCCGGGCGGCTCGCTGCTGCCC
CCTAGCGGGGGAGGGACGTAATTACATCCCTGGGGGCTTTGGGGGGGGGCTGTCCCCGTGAGCTC
Modified HS4-650 insulator (r-c)-A to T mutation at SA2 (mutations in bold and underlined)
(SEQ ID NO: 7)
AGCCCCATCCTCACTGACTCCGTCCTGGAGTTGGATGAGAGATAATGGCCTTACGTTGTGCCAGGGG
AGGGTCGGGCTGGATTTAGCAAGATTTACCTTCTCCAAAGAGCGGTGCTGCAGTGGCACAGCTGCCC
ACGGAGGTGGGGGGGTCACCGTCCCTGGAGGTGATGAAGAACTGTGGGGATGTGGCACTGAGGGA
CATGGCCAGTGGGCACGGTGGGTGGGTTGGGGTTGGTCTTGGGGATCTTGGAGGGCTTTTCCAGCC
TTCATGATTTGACGATTGTATGAACATCTACATGGCAATTCTCCAGCTGCCTGTCCCAGTCCTACTGAC
CCAGCTGTATCTCTCCAGGCAAGCTCTTCCACCCCTTCTGCTTGCATCCAGACACCATCAAACATGCA
GGCTCAGACACATGATATCAAGCTTTTTCCCCGTATCCCCCCTGGTGTCTGCAGGCTCAAAGAGCAGC
GAGAAGCGTTCAGAGGAAAGCGATCCCGTGCCACCTTCCCCGTGCCCGGGCTGTCCCCGCACGCTG
CCGGCTCGGGGATGCGGGGGGAGCGCCGGACCGGAGCGGAGCCCCGGGCGGCTCGCTGCTGCCC
CCTAGCGGGGGAGGGACGTAATTACATCCCTGGGGGCTTTGGGGGGGGGCTGTCCCCGTGAGCTC
Modified HS4-650 insulator (r-c)-A to T mutations at SA2 and SA3 (mutations in bold and
underlined) (SEQ ID NO: 8)
AGCCCCATCCTCACTGACTCCGTCCTGGAGTTGGATGAGAGATAATGGCCTTACGTTGTGCCAGGGG
AGGGTCGGGCTGGATTTAGCAAGATTTACCTTCTCCAAAGAGCGGTGCTGCAGTGGCACAGCTGCCC
ACGGAGGTGGGGGGGTCACCGTCCCTGGAGGTGATGAAGAACTGTGGGGATGTGGCACTGAGGGA
CATGGCCAGTGGGCACGGTGGGTGGGTTGGGGTTGGTCTTGGGGATCTTGGAGGGCTTTTCCAGCC
TTCATGATTTGACGATTGTATGAACATCTACATGGCAATTCTCCAGCTGCCTGTCCCAGTCCTACTGAC
CCAGCTGTATCTCTCCAGGCAAGCTCTTCCACCCCTTCTGCTTGCATCCAGACACCATCAAACATGCA
GGCTCAGACACATGATATCAAGCTTTTTCCCCGTATCCCCCCTGGTGTCTGCTGGCTCAAAGAGCAGC
GAGAAGCGTTCAGAGGAAAGCGATCCCGTGCCACCTTCCCCGTGCCCGGGCTGTCCCCGCACGCTG
CCGGCTCGGGGATGCGGGGGGAGCGCCGGACCGGAGCGGAGCCCCGGGCGGCTCGCTGCTGCCC
CCTAGCGGGGGAGGGACGTAATTACATCCCTGGGGGCTTTGGGGGGGGGCTGTCCCCGTGAGCTC
Modified HS4-650 insulator (r-c)-A to T mutation at SA3 (mutations in bold and underlined)
(SEQ ID NO: 9)
AGCCCCATCCTCACTGACTCCGTCCTGGAGTTGGATGAGAGATAATGGCCTTACGTTGTGCCAGGGG
AGGGTCGGGCTGGATTTAGCAAGATTTACCTTCTCCAAAGAGCGGTGCTGCAGTGGCACAGCTGCCC
ACGGAGGTGGGGGGGTCACCGTCCCTGGAGGTGATGAAGAACTGTGGGGATGTGGCACTGAGGGA
CATGGCCAGTGGGCACGGTGGGTGGGTTGGGGTTGGTCTTGGGGATCTTGGAGGGCTTTTCCAGCC
TTCATGATTTGACGATTGTATGAACATCTACATGGCAATTCTCCAGCTGCCTGTCCCAGTCCTACTGAC
CCAGCTGTATCTCTCCAGGCAAGCTCTTCCACCCCTTCTGCTTGCATCCAGACACCATCAAACATGCA
GGCTCAGACACATGATATCAAGCTTTTTCCCCGTATCCCCCCAGGTGTCTGCTGGCTCAAAGAGCAGC
GAGAAGCGTTCAGAGGAAAGCGATCCCGTGCCACCTTCCCCGTGCCCGGGCTGTCCCCGCACGCTG
CCGGCTCGGGGATGCGGGGGGAGCGCCGGACCGGAGCGGAGCCCCGGGCGGCTCGCTGCTGCCC
CCTAGCGGGGGAGGGACGTAATTACATCCCTGGGGGCTTTGGGGGGGGGCTGTCCCCGTGAGCTC
Unmodified HS4-650 insulator (Genbank Acc. No. JN000001) (SEQ ID NO: 10)
ACGGGGACAGCCCCCCCCCAAAGCCCCCAGGGATGTAATTACGTCCCTCCCCCGCTAGGGGGCAGC
AGCGAGCCGCCCGGGGCTCCGCTCCGGTCCGGCGCTCCCCCCGCATCCCCGAGCCGGCAGCGTGC
GGGGACAGCCCGGGCACGGGGAAGGTGGCACGGGATCGCTTTCCTCTGAACGCTTCTCGCTGCTCT
TTGAGCCTGCAGACACCTGGGGGGATACGGGGAAAAAGCTTTAGGCTTGTGTCTGAGCCTGCATGTT
TGATGGTGTCTGGATGCAAGCAGAAGGGGTGGAAGAGCTTGCCTGGAGAGATACAGCTGGGTCAGT
AGGACTGGGACAGGCAGCTGGAGAATTGCCATGTAGATGTTCATACAATCGTCAAATCATGAAGGCT
GGAAAAGCCCTCCAAGATCCCCAAGACCAACCCCAACCCACCCACCGTGCCCACTGGCCATGTCCCT
CAGTGCCACATCCCCACAGTTCTTCATCACCTCCAGGGACGGTGACCCCCCCACCTCCGTGGGCAGC
TGTGCCACTGCAGCACCGCTCTTTGGAGAAGGTAAATCTTGCTAAATCCAGCCCGACCCTCCCCTGGC
ACAACGTAAGGCCATTATCTCTCATCCAACTCCAGGACGGAGTCAGTGAG
Reverse complement (r-c) unmodified HS4-650 insulator (Genbank Acc. No. JN000001) (SEQ
ID NO: 11)
CTCACTGACTCCGTCCTGGAGTTGGATGAGAGATAATGGCCTTACGTTGTGCCAGGGGAGGGTCGGG
CTGGATTTAGCAAGATTTACCTTCTCCAAAGAGCGGTGCTGCAGTGGCACAGCTGCCCACGGAGGTG
GGGGGGTCACCGTCCCTGGAGGTGATGAAGAACTGTGGGGATGTGGCACTGAGGGACATGGCCAGT
GGGCACGGTGGGTGGGTTGGGGTTGGTCTTGGGGATCTTGGAGGGCTTTTCCAGCCTTCATGATTTG
ACGATTGTATGAACATCTACATGGCAATTCTCCAGCTGCCTGTCCCAGTCCTACTGACCCAGCTGTAT
CTCTCCAGGCAAGCTCTTCCACCCCTTCTGCTTGCATCCAGACACCATCAAACATGCAGGCTCAGACA
CAAGCCTAAAGCTTTTTCCCCGTATCCCCCCAGGTGTCTGCAGGCTCAAAGAGCAGCGAGAAGCGTT
CAGAGGAAAGCGATCCCGTGCCACCTTCCCCGTGCCCGGGCTGTCCCCGCACGCTGCCGGCTCGGG
GATGCGGGGGGAGCGCCGGACCGGAGCGGAGCCCCGGGCGGCTCGCTGCTGCCCCCTAGCGGGG
GAGGGACGTAATTACATCCCTGGGGGCTTTGGGGGGGGGCTGTCCCCGT
Modified HS4-650 insulator (Genbank Acc. No. JN000001) (r-c)-A to T mutation at SA1
(mutation in bold and underlined) (SEQ ID NO: 12)
CTCACTGACTCCGTCCTGGAGTTGGATGAGAGATAATGGCCTTACGTTGTGCCAGGGGAGGGTCGGG
CTGGATTTAGCAAGATTTACCTTCTCCAAAGAGCGGTGCTGCAGTGGCACAGCTGCCCACGGAGGTG
GGGGGGTCACCGTCCCTGGAGGTGATGAAGAACTGTGGGGATGTGGCACTGAGGGACATGGCCAGT
GGGCACGGTGGGTGGGTTGGGGTTGGTCTTGGGGATCTTGGAGGGCTTTTCCAGCCTTCATGATTTG
ACGATTGTATGAACATCTACATGGCAATTCTCCAGCTGCCTGTCCCAGTCCTACTGACCCAGCTGTAT
CTCTCCAGGCAAGCTCTTCCACCCCTTCTGCTTGCATCCTGACACCATCAAACATGCAGGCTCAGACA
CAAGCCTAAAGCTTTTTCCCCGTATCCCCCCAGGTGTCTGCAGGCTCAAAGAGCAGCGAGAAGCGTT
CAGAGGAAAGCGATCCCGTGCCACCTTCCCCGTGCCCGGGCTGTCCCCGCACGCTGCCGGCTCGGG
GATGCGGGGGGAGCGCCGGACCGGAGCGGAGCCCCGGGCGGCTCGCTGCTGCCCCCTAGCGGGG
GAGGGACGTAATTACATCCCTGGGGGCTTTGGGGGGGGGCTGTCCCCGT
Modified HS4-650 insulator (Genbank Acc. No. JN000001) (r-c)-A to T mutation at SA1 and
SA2 (mutations in bold and underlined) (SEQ ID NO: 13)
CTCACTGACTCCGTCCTGGAGTTGGATGAGAGATAATGGCCTTACGTTGTGCCAGGGGAGGGTCGGG
CTGGATTTAGCAAGATTTACCTTCTCCAAAGAGCGGTGCTGCAGTGGCACAGCTGCCCACGGAGGTG
GGGGGGTCACCGTCCCTGGAGGTGATGAAGAACTGTGGGGATGTGGCACTGAGGGACATGGCCAGT
GGGCACGGTGGGTGGGTTGGGGTTGGTCTTGGGGATCTTGGAGGGCTTTTCCAGCCTTCATGATTTG
ACGATTGTATGAACATCTACATGGCAATTCTCCAGCTGCCTGTCCCAGTCCTACTGACCCAGCTGTAT
CTCTCCAGGCAAGCTCTTCCACCCCTTCTGCTTGCATCCTGACACCATCAAACATGCAGGCTCAGACA
CAAGCCTAAAGCTTTTTCCCCGTATCCCCCCTGGTGTCTGCAGGCTCAAAGAGCAGCGAGAAGCGTT
CAGAGGAAAGCGATCCCGTGCCACCTTCCCCGTGCCCGGGCTGTCCCCGCACGCTGCCGGCTCGGG
GATGCGGGGGGAGCGCCGGACCGGAGCGGAGCCCCGGGCGGCTCGCTGCTGCCCCCTAGCGGGG
GAGGGACGTAATTACATCCCTGGGGGCTTTGGGGGGGGGCTGTCCCCGT
Modified HS4-650 insulator (Genbank Acc. No. JN000001) (r-c)-A to T mutation at SA1 and
SA3 (mutations in bold and underlined) (SEQ ID NO: 14)
CTCACTGACTCCGTCCTGGAGTTGGATGAGAGATAATGGCCTTACGTTGTGCCAGGGGAGGGTCGGG
CTGGATTTAGCAAGATTTACCTTCTCCAAAGAGCGGTGCTGCAGTGGCACAGCTGCCCACGGAGGTG
GGGGGGTCACCGTCCCTGGAGGTGATGAAGAACTGTGGGGATGTGGCACTGAGGGACATGGCCAGT
GGGCACGGTGGGTGGGTTGGGGTTGGTCTTGGGGATCTTGGAGGGCTTTTCCAGCCTTCATGATTTG
ACGATTGTATGAACATCTACATGGCAATTCTCCAGCTGCCTGTCCCAGTCCTACTGACCCAGCTGTAT
CTCTCCAGGCAAGCTCTTCCACCCCTTCTGCTTGCATCCTGACACCATCAAACATGCAGGCTCAGACA
CAAGCCTAAAGCTTTTTCCCCGTATCCCCCCAGGTGTCTGCTGGCTCAAAGAGCAGCGAGAAGCGTT
CAGAGGAAAGCGATCCCGTGCCACCTTCCCCGTGCCCGGGCTGTCCCCGCACGCTGCCGGCTCGGG
GATGCGGGGGGAGCGCCGGACCGGAGCGGAGCCCCGGGCGGCTCGCTGCTGCCCCCTAGCGGGG
GAGGGACGTAATTACATCCCTGGGGGCTTTGGGGGGGGGCTGTCCCCGT
Modified HS4-650 insulator (Genbank Acc. No. JN000001) (r-c)-A to T mutation at SA1,
SA2 and SA3 (mutations in bold and underlined) (SEQ ID NO: 15)
CTCACTGACTCCGTCCTGGAGTTGGATGAGAGATAATGGCCTTACGTTGTGCCAGGGGAGGGTCGGG
CTGGATTTAGCAAGATTTACCTTCTCCAAAGAGCGGTGCTGCAGTGGCACAGCTGCCCACGGAGGTG
GGGGGGTCACCGTCCCTGGAGGTGATGAAGAACTGTGGGGATGTGGCACTGAGGGACATGGCCAGT
GGGCACGGTGGGTGGGTTGGGGTTGGTCTTGGGGATCTTGGAGGGCTTTTCCAGCCTTCATGATTTG
ACGATTGTATGAACATCTACATGGCAATTCTCCAGCTGCCTGTCCCAGTCCTACTGACCCAGCTGTAT
CTCTCCAGGCAAGCTCTTCCACCCCTTCTGCTTGCATCCTGACACCATCAAACATGCAGGCTCAGACA
CAAGCCTAAAGCTTTTTCCCCGTATCCCCCCTGGTGTCTGCTGGCTCAAAGAGCAGCGAGAAGCGTT
CAGAGGAAAGCGATCCCGTGCCACCTTCCCCGTGCCCGGGCTGTCCCCGCACGCTGCCGGCTCGGG
GATGCGGGGGGAGCGCCGGACCGGAGCGGAGCCCCGGGCGGCTCGCTGCTGCCCCCTAGCGGGG
GAGGGACGTAATTACATCCCTGGGGGCTTTGGGGGGGGGCTGTCCCCGT
Modified HS4-650 insulator (Genbank Acc. No. JN000001) (r-c)-A to T mutation and SA2
(mutation in bold and underlined) (SEQ ID NO: 16)
CTCACTGACTCCGTCCTGGAGTTGGATGAGAGATAATGGCCTTACGTTGTGCCAGGGGAGGGTCGGG
CTGGATTTAGCAAGATTTACCTTCTCCAAAGAGCGGTGCTGCAGTGGCACAGCTGCCCACGGAGGTG
GGGGGGTCACCGTCCCTGGAGGTGATGAAGAACTGTGGGGATGTGGCACTGAGGGACATGGCCAGT
GGGCACGGTGGGTGGGTTGGGGTTGGTCTTGGGGATCTTGGAGGGCTTTTCCAGCCTTCATGATTTG
ACGATTGTATGAACATCTACATGGCAATTCTCCAGCTGCCTGTCCCAGTCCTACTGACCCAGCTGTAT
CTCTCCAGGCAAGCTCTTCCACCCCTTCTGCTTGCATCCAGACACCATCAAACATGCAGGCTCAGACA
CAAGCCTAAAGCTTTTTCCCCGTATCCCCCCTGGTGTCTGCAGGCTCAAAGAGCAGCGAGAAGCGTT
CAGAGGAAAGCGATCCCGTGCCACCTTCCCCGTGCCCGGGCTGTCCCCGCACGCTGCCGGCTCGGG
GATGCGGGGGGAGCGCCGGACCGGAGCGGAGCCCCGGGCGGCTCGCTGCTGCCCCCTAGCGGGG
GAGGGACGTAATTACATCCCTGGGGGCTTTGGGGGGGGGCTGTCCCCGT
Modified HS4-650 insulator (Genbank Acc. No. JN000001) (r-c)-A to T mutation at SA2 and
SA3 (mutations in bold and underlined) (SEQ ID NO: 17)
CTCACTGACTCCGTCCTGGAGTTGGATGAGAGATAATGGCCTTACGTTGTGCCAGGGGAGGGTCGGG
CTGGATTTAGCAAGATTTACCTTCTCCAAAGAGCGGTGCTGCAGTGGCACAGCTGCCCACGGAGGTG
GGGGGGTCACCGTCCCTGGAGGTGATGAAGAACTGTGGGGATGTGGCACTGAGGGACATGGCCAGT
GGGCACGGTGGGTGGGTTGGGGTTGGTCTTGGGGATCTTGGAGGGCTTTTCCAGCCTTCATGATTTG
ACGATTGTATGAACATCTACATGGCAATTCTCCAGCTGCCTGTCCCAGTCCTACTGACCCAGCTGTAT
CTCTCCAGGCAAGCTCTTCCACCCCTTCTGCTTGCATCCAGACACCATCAAACATGCAGGCTCAGACA
CAAGCCTAAAGCTTTTTCCCCGTATCCCCCCTGGTGTCTGCTGGCTCAAAGAGCAGCGAGAAGCGTT
CAGAGGAAAGCGATCCCGTGCCACCTTCCCCGTGCCCGGGCTGTCCCCGCACGCTGCCGGCTCGGG
GATGCGGGGGGAGCGCCGGACCGGAGCGGAGCCCCGGGCGGCTCGCTGCTGCCCCCTAGCGGGG
GAGGGACGTAATTACATCCCTGGGGGCTTTGGGGGGGGGCTGTCCCCGT
Modified HS4-650 insulator (Genbank Acc. No. JN000001) (r-c)-A to T mutation at SA3
(mutation in bold and underlined) (SEQ ID NO: 18)
CTCACTGACTCCGTCCTGGAGTTGGATGAGAGATAATGGCCTTACGTTGTGCCAGGGGAGGGTCGGG
CTGGATTTAGCAAGATTTACCTTCTCCAAAGAGCGGTGCTGCAGTGGCACAGCTGCCCACGGAGGTG
GGGGGGTCACCGTCCCTGGAGGTGATGAAGAACTGTGGGGATGTGGCACTGAGGGACATGGCCAGT
GGGCACGGTGGGTGGGTTGGGGTTGGTCTTGGGGATCTTGGAGGGCTTTTCCAGCCTTCATGATTTG
ACGATTGTATGAACATCTACATGGCAATTCTCCAGCTGCCTGTCCCAGTCCTACTGACCCAGCTGTAT
CTCTCCAGGCAAGCTCTTCCACCCCTTCTGCTTGCATCCAGACACCATCAAACATGCAGGCTCAGACA
CAAGCCTAAAGCTTTTTCCCCGTATCCCCCCAGGTGTCTGCTGGCTCAAAGAGCAGCGAGAAGCGTT
CAGAGGAAAGCGATCCCGTGCCACCTTCCCCGTGCCCGGGCTGTCCCCGCACGCTGCCGGCTCGGG
GATGCGGGGGGAGCGCCGGACCGGAGCGGAGCCCCGGGCGGCTCGCTGCTGCCCCCTAGCGGGG
GAGGGACGTAATTACATCCCTGGGGGCTTTGGGGGGGGGCTGTCCCCGT
Unmodified HS4-650 insulator (US2016003218) (SEQ ID NO: 19)
AAGATCTGCTCACGGGGACAGCCCCCCCCCAAAGCCCCCAGGGATGTAATTACGTCCCTCCCCCGCT
AGGGGGCAGCAGCGAGCCGCCCGGGGCTCCGCTCCGGTCCGGCGCTCCCCCCGCATCCCCGAGCC
GGCAGCGTGCGGGGACAGCCCGGGCACGGGGAAGGTGGCACGGGATCGCTTTCCTCTGAACGCTTC
TCGCTGCTCTTTGAGCCTGCAGACACCTGGGGGGATACGGGGAAAATGTGTCTGAGCCTGCATGTTT
GATGGTGTCTGGATGCAAGCAGAAGGGGTGGAAGAGCTTGCCTGGAGAGATACAGCTGGGTCAGTA
GGACTGGGACAGGCAGCTGGAGAATTGCCATGTAGATGTTCATACAATCGTCAAATCATGAAGGCTG
GAAAAGCCCTCCAAGATCCCCAAGACCAACCCCAACCCACCCACCGTGCCCACTGGCCATGTCCCTC
AGTGCCACATCCCCACAGTTCTTCATCACCTCCAGGGACGGTGACCCCCCCACCTCCGTGGGCAGCT
GTGCCACTGCAGCACCGCTCTTTGGAGAAGGTAAATCTTGCTAAATCCAGCCCGACCCTCCCCTGGC
ACAACGTAAGGCCATTATCTCTCATCCAACTCCAGGACGGAGTCAGTGAGAATATT
Reverse complement of unmodified HS4-650 insulator (US2016003218) (SEQ ID NO: 20)
AATATTCTCACTGACTCCGTCCTGGAGTTGGATGAGAGATAATGGCCTTACGTTGTGCCAGGGGAGG
GTCGGGCTGGATTTAGCAAGATTTACCTTCTCCAAAGAGCGGTGCTGCAGTGGCACAGCTGCCCACG
GAGGTGGGGGGGTCACCGTCCCTGGAGGTGATGAAGAACTGTGGGGATGTGGCACTGAGGGACAT
GGCCAGTGGGCACGGTGGGTGGGTTGGGGTTGGTCTTGGGGATCTTGGAGGGCTTTTCCAGCCTTC
ATGATTTGACGATTGTATGAACATCTACATGGCAATTCTCCAGCTGCCTGTCCCAGTCCTACTGACCCA
GCTGTATCTCTCCAGGCAAGCTCTTCCACCCCTTCTGCTTGCATCCAGACACCATCAAACATGCAGGC
TCAGACACATTTTCCCCGTATCCCCCCAGGTGTCTGCAGGCTCAAAGAGCAGCGAGAAGCGTTCAGA
GGAAAGCGATCCCGTGCCACCTTCCCCGTGCCCGGGCTGTCCCCGCACGCTGCCGGCTCGGGGATG
CGGGGGGAGCGCCGGACCGGAGCGGAGCCCCGGGCGGCTCGCTGCTGCCCCCTAGCGGGGGAGG
GACGTAATTACATCCCTGGGGGCTTTGGGGGGGGGCTGTCCCCGTGAGCAGATCTT
Modified HS4-650 insulator (US2016003218) (r-c)-A to T mutation at SA1 (mutation in bold
and underlined) (SEQ ID NO: 21)
AATATTCTCACTGACTCCGTCCTGGAGTTGGATGAGAGATAATGGCCTTACGTTGTGCCAGGGGAGG
GTCGGGCTGGATTTAGCAAGATTTACCTTCTCCAAAGAGCGGTGCTGCAGTGGCACAGCTGCCCACG
GAGGTGGGGGGGTCACCGTCCCTGGAGGTGATGAAGAACTGTGGGGATGTGGCACTGAGGGACAT
GGCCAGTGGGCACGGTGGGTGGGTTGGGGTTGGTCTTGGGGATCTTGGAGGGCTTTTCCAGCCTTC
ATGATTTGACGATTGTATGAACATCTACATGGCAATTCTCCAGCTGCCTGTCCCAGTCCTACTGACCCA
GCTGTATCTCTCCAGGCAAGCTCTTCCACCCCTTCTGCTTGCATCCTGACACCATCAAACATGCAGGC
TCAGACACATTTTCCCCGTATCCCCCCAGGTGTCTGCAGGCTCAAAGAGCAGCGAGAAGCGTTCAGA
GGAAAGCGATCCCGTGCCACCTTCCCCGTGCCCGGGCTGTCCCCGCACGCTGCCGGCTCGGGGATG
CGGGGGGAGCGCCGGACCGGAGCGGAGCCCCGGGCGGCTCGCTGCTGCCCCCTAGCGGGGGAGG
GACGTAATTACATCCCTGGGGGCTTTGGGGGGGGGCTGTCCCCGTGAGCAGATCTT
Modified HS4-650 insulator (US2016003218) (r-c)-A to T mutation at SA1 and SA2
(mutations in bold and underlined) (SEQ ID NO: 22)
AATATTCTCACTGACTCCGTCCTGGAGTTGGATGAGAGATAATGGCCTTACGTTGTGCCAGGGGAGG
GTCGGGCTGGATTTAGCAAGATTTACCTTCTCCAAAGAGCGGTGCTGCAGTGGCACAGCTGCCCACG
GAGGTGGGGGGGTCACCGTCCCTGGAGGTGATGAAGAACTGTGGGGATGTGGCACTGAGGGACAT
GGCCAGTGGGCACGGTGGGTGGGTTGGGGTTGGTCTTGGGGATCTTGGAGGGCTTTTCCAGCCTTC
ATGATTTGACGATTGTATGAACATCTACATGGCAATTCTCCAGCTGCCTGTCCCAGTCCTACTGACCCA
GCTGTATCTCTCCAGGCAAGCTCTTCCACCCCTTCTGCTTGCATCCTGACACCATCAAACATGCAGGC
TCAGACACATTTTCCCCGTATCCCCCCTGGTGTCTGCAGGCTCAAAGAGCAGCGAGAAGCGTTCAGA
GGAAAGCGATCCCGTGCCACCTTCCCCGTGCCCGGGCTGTCCCCGCACGCTGCCGGCTCGGGGATG
CGGGGGGAGCGCCGGACCGGAGCGGAGCCCCGGGCGGCTCGCTGCTGCCCCCTAGCGGGGGAGG
GACGTAATTACATCCCTGGGGGCTTTGGGGGGGGGCTGTCCCCGTGAGCAGATCTT
Modified HS4-650 insulator (US2016003218) (r-c)-A to T mutation at SA1 and SA3
(mutations in bold and underlined) (SEQ ID NO: 23)
AATATTCTCACTGACTCCGTCCTGGAGTTGGATGAGAGATAATGGCCTTACGTTGTGCCAGGGGAGG
GTCGGGCTGGATTTAGCAAGATTTACCTTCTCCAAAGAGCGGTGCTGCAGTGGCACAGCTGCCCACG
GAGGTGGGGGGGTCACCGTCCCTGGAGGTGATGAAGAACTGTGGGGATGTGGCACTGAGGGACAT
GGCCAGTGGGCACGGTGGGTGGGTTGGGGTTGGTCTTGGGGATCTTGGAGGGCTTTTCCAGCCTTC
ATGATTTGACGATTGTATGAACATCTACATGGCAATTCTCCAGCTGCCTGTCCCAGTCCTACTGACCCA
GCTGTATCTCTCCAGGCAAGCTCTTCCACCCCTTCTGCTTGCATCCTGACACCATCAAACATGCAGGC
TCAGACACATTTTCCCCGTATCCCCCCAGGTGTCTGCTGGCTCAAAGAGCAGCGAGAAGCGTTCAGA
GGAAAGCGATCCCGTGCCACCTTCCCCGTGCCCGGGCTGTCCCCGCACGCTGCCGGCTCGGGGATG
CGGGGGGAGCGCCGGACCGGAGCGGAGCCCCGGGCGGCTCGCTGCTGCCCCCTAGCGGGGGAGG
GACGTAATTACATCCCTGGGGGCTTTGGGGGGGGGCTGTCCCCGTGAGCAGATCTT
Modified HS4-650 insulator (US2016003218) (r-c)-A to T mutation at SA1, SA2 and SA3
(mutations in bold and underlined) (SEQ ID NO: 24)
AATATTCTCACTGACTCCGTCCTGGAGTTGGATGAGAGATAATGGCCTTACGTTGTGCCAGGGGAGG
GTCGGGCTGGATTTAGCAAGATTTACCTTCTCCAAAGAGCGGTGCTGCAGTGGCACAGCTGCCCACG
GAGGTGGGGGGGTCACCGTCCCTGGAGGTGATGAAGAACTGTGGGGATGTGGCACTGAGGGACAT
GGCCAGTGGGCACGGTGGGTGGGTTGGGGTTGGTCTTGGGGATCTTGGAGGGCTTTTCCAGCCTTC
ATGATTTGACGATTGTATGAACATCTACATGGCAATTCTCCAGCTGCCTGTCCCAGTCCTACTGACCCA
GCTGTATCTCTCCAGGCAAGCTCTTCCACCCCTTCTGCTTGCATCCTGACACCATCAAACATGCAGGC
TCAGACACATTTTCCCCGTATCCCCCCTGGTGTCTGCTGGCTCAAAGAGCAGCGAGAAGCGTTCAGA
GGAAAGCGATCCCGTGCCACCTTCCCCGTGCCCGGGCTGTCCCCGCACGCTGCCGGCTCGGGGATG
CGGGGGGAGCGCCGGACCGGAGCGGAGCCCCGGGCGGCTCGCTGCTGCCCCCTAGCGGGGGAGG
GACGTAATTACATCCCTGGGGGCTTTGGGGGGGGGCTGTCCCCGTGAGCAGATCTT
Modified HS4-650 insulator (US2016003218) (r-c)-A to T mutation at SA2 (mutation in bold
and underlined) (SEQ ID NO: 25)
AATATTCTCACTGACTCCGTCCTGGAGTTGGATGAGAGATAATGGCCTTACGTTGTGCCAGGGGAGG
GTCGGGCTGGATTTAGCAAGATTTACCTTCTCCAAAGAGCGGTGCTGCAGTGGCACAGCTGCCCACG
GAGGTGGGGGGGTCACCGTCCCTGGAGGTGATGAAGAACTGTGGGGATGTGGCACTGAGGGACAT
GGCCAGTGGGCACGGTGGGTGGGTTGGGGTTGGTCTTGGGGATCTTGGAGGGCTTTTCCAGCCTTC
ATGATTTGACGATTGTATGAACATCTACATGGCAATTCTCCAGCTGCCTGTCCCAGTCCTACTGACCCA
GCTGTATCTCTCCAGGCAAGCTCTTCCACCCCTTCTGCTTGCATCCAGACACCATCAAACATGCAGGC
TCAGACACATTTTCCCCGTATCCCCCCTGGTGTCTGCAGGCTCAAAGAGCAGCGAGAAGCGTTCAGA
GGAAAGCGATCCCGTGCCACCTTCCCCGTGCCCGGGCTGTCCCCGCACGCTGCCGGCTCGGGGATG
CGGGGGGAGCGCCGGACCGGAGCGGAGCCCCGGGCGGCTCGCTGCTGCCCCCTAGCGGGGGAGG
GACGTAATTACATCCCTGGGGGCTTTGGGGGGGGGCTGTCCCCGTGAGCAGATCTT
Modified HS4-650 insulator (US2016003218) (r-c)-A to T mutation at SA2 and SA3
(mutations in bold and underlined) (SEQ ID NO: 26)
AATATTCTCACTGACTCCGTCCTGGAGTTGGATGAGAGATAATGGCCTTACGTTGTGCCAGGGGAGG
GTCGGGCTGGATTTAGCAAGATTTACCTTCTCCAAAGAGCGGTGCTGCAGTGGCACAGCTGCCCACG
GAGGTGGGGGGGTCACCGTCCCTGGAGGTGATGAAGAACTGTGGGGATGTGGCACTGAGGGACAT
GGCCAGTGGGCACGGTGGGTGGGTTGGGGTTGGTCTTGGGGATCTTGGAGGGCTTTTCCAGCCTTC
ATGATTTGACGATTGTATGAACATCTACATGGCAATTCTCCAGCTGCCTGTCCCAGTCCTACTGACCCA
GCTGTATCTCTCCAGGCAAGCTCTTCCACCCCTTCTGCTTGCATCCAGACACCATCAAACATGCAGGC
TCAGACACATTTTCCCCGTATCCCCCCTGGTGTCTGCTGGCTCAAAGAGCAGCGAGAAGCGTTCAGA
GGAAAGCGATCCCGTGCCACCTTCCCCGTGCCCGGGCTGTCCCCGCACGCTGCCGGCTCGGGGATG
CGGGGGGAGCGCCGGACCGGAGCGGAGCCCCGGGCGGCTCGCTGCTGCCCCCTAGCGGGGGAGG
GACGTAATTACATCCCTGGGGGCTTTGGGGGGGGGCTGTCCCCGTGAGCAGATCTT
Modified HS4-650 insulator (US2016003218) (r-c)-A to T mutation at SA3 (mutations in
bold and underlined) (SEQ ID NO: 27)
AATATTCTCACTGACTCCGTCCTGGAGTTGGATGAGAGATAATGGCCTTACGTTGTGCCAGGGGAGG
GTCGGGCTGGATTTAGCAAGATTTACCTTCTCCAAAGAGCGGTGCTGCAGTGGCACAGCTGCCCACG
GAGGTGGGGGGGTCACCGTCCCTGGAGGTGATGAAGAACTGTGGGGATGTGGCACTGAGGGACAT
GGCCAGTGGGCACGGTGGGTGGGTTGGGGTTGGTCTTGGGGATCTTGGAGGGCTTTTCCAGCCTTC
ATGATTTGACGATTGTATGAACATCTACATGGCAATTCTCCAGCTGCCTGTCCCAGTCCTACTGACCCA
GCTGTATCTCTCCAGGCAAGCTCTTCCACCCCTTCTGCTTGCATCCAGACACCATCAAACATGCAGGC
TCAGACACATTTTCCCCGTATCCCCCCAGGTGTCTGCTGGCTCAAAGAGCAGCGAGAAGCGTTCAGA
GGAAAGCGATCCCGTGCCACCTTCCCCGTGCCCGGGCTGTCCCCGCACGCTGCCGGCTCGGGGATG
CGGGGGGAGCGCCGGACCGGAGCGGAGCCCCGGGCGGCTCGCTGCTGCCCCCTAGCGGGGGAGG
GACGTAATTACATCCCTGGGGGCTTTGGGGGGGGGCTGTCCCCGTGAGCAGATCTT
Unmodified HS4-650 insulator (Genbank Acc. No. MN044710.1) (SEQ ID NO: 28)
CCTGTACGGGGACAGCCCCCCCCCAAAGCCCCCAGGGATGTAATTACGTCCCTCCCCCGCTAGGGG
GCAGCAGCGAGCCGCCCGGGGCTCCGCTCCGGTCCGGCGCTCCCCCCGCATCCCCGAGCCGGCAG
CGTGCGGGGACAGCCCGGGCACGGGGAAGGTGGCACGGGATCGCTTTCCTCTGAACGCTTCTCGCT
GCTCTTTGAGCCTGCAGACACCTGGGGGGATACGGGGAAATGTGTCTGAGCCTGCATGTTTGATGGT
GTCTGGATGCAAGCAGAAGGGGTGGAAGAGCTTGCCTGGAGAGATACAGCTGGGTCAGTAGGACTG
GGACAGGCAGCTGGAGAATTGCCATGTAGATGTTCATACAATCGTCAAATCATGAAGGCTGGAAAAG
CCCTCCAAGATCCCCAAGACCAACCCCAACCCACCCACCGTGCCCACTGGCCATGTCCCTCAGTGCC
ACATCCCCACAGTTCTTCATCACCTCCAGGGACGGTGACCCCCCCACCTCCGTGGGCAGCTGTGCCA
CTGCAGCACCGCTCTTTGGAGAAGGTAAATCTTGCTAAATCCAGCCCGACCCTCCCCTGGCACAACGT
AAGGCCATTATCTCTCATCCAACTCCAGGACGGAGTCAGTGAGGATGGGGCT
Reverse complement of unmodified HS4-650 insulator (Genbank Acc. No. MN044710.1) (SEQ
ID NO: 29)
AGCCCCATCCTCACTGACTCCGTCCTGGAGTTGGATGAGAGATAATGGCCTTACGTTGTGCCAGGGG
AGGGTCGGGCTGGATTTAGCAAGATTTACCTTCTCCAAAGAGCGGTGCTGCAGTGGCACAGCTGCCC
ACGGAGGTGGGGGGGTCACCGTCCCTGGAGGTGATGAAGAACTGTGGGGATGTGGCACTGAGGGA
CATGGCCAGTGGGCACGGTGGGTGGGTTGGGGTTGGTCTTGGGGATCTTGGAGGGCTTTTCCAGCC
TTCATGATTTGACGATTGTATGAACATCTACATGGCAATTCTCCAGCTGCCTGTCCCAGTCCTACTGAC
CCAGCTGTATCTCTCCAGGCAAGCTCTTCCACCCCTTCTGCTTGCATCCAGACACCATCAAACATGCA
GGCTCAGACACATTTCCCCGTATCCCCCCAGGTGTCTGCAGGCTCAAAGAGCAGCGAGAAGCGTTCA
GAGGAAAGCGATCCCGTGCCACCTTCCCCGTGCCCGGGCTGTCCCCGCACGCTGCCGGCTCGGGGA
TGCGGGGGGAGCGCCGGACCGGAGCGGAGCCCCGGGCGGCTCGCTGCTGCCCCCTAGCGGGGGA
GGGACGTAATTACATCCCTGGGGGCTTTGGGGGGGGGCTGTCCCCGTACAGG
Modified HS4-650 insulator (Genbank Acc. No. MNO44710.1) (r-c)-A to T mutation at SA1
(mutation in bold and underlined) (SEQ ID NO: 30)
AGCCCCATCCTCACTGACTCCGTCCTGGAGTTGGATGAGAGATAATGGCCTTACGTTGTGCCAGGGG
AGGGTCGGGCTGGATTTAGCAAGATTTACCTTCTCCAAAGAGCGGTGCTGCAGTGGCACAGCTGCCC
ACGGAGGTGGGGGGGTCACCGTCCCTGGAGGTGATGAAGAACTGTGGGGATGTGGCACTGAGGGA
CATGGCCAGTGGGCACGGTGGGTGGGTTGGGGTTGGTCTTGGGGATCTTGGAGGGCTTTTCCAGCC
TTCATGATTTGACGATTGTATGAACATCTACATGGCAATTCTCCAGCTGCCTGTCCCAGTCCTACTGAC
CCAGCTGTATCTCTCCAGGCAAGCTCTTCCACCCCTTCTGCTTGCATCCTGACACCATCAAACATGCA
GGCTCAGACACATTTCCCCGTATCCCCCCAGGTGTCTGCAGGCTCAAAGAGCAGCGAGAAGCGTTCA
GAGGAAAGCGATCCCGTGCCACCTTCCCCGTGCCCGGGCTGTCCCCGCACGCTGCCGGCTCGGGGA
TGCGGGGGGAGCGCCGGACCGGAGCGGAGCCCCGGGCGGCTCGCTGCTGCCCCCTAGCGGGGGA
GGGACGTAATTACATCCCTGGGGGCTTTGGGGGGGGGCTGTCCCCGTACAGG
Modified HS4-650 insulator (Genbank Acc. No. MNO44710.1) (r-c)-A to T mutation at SA1
and SA2 (mutations in bold and underlined) (SEQ ID NO: 31)
AGCCCCATCCTCACTGACTCCGTCCTGGAGTTGGATGAGAGATAATGGCCTTACGTTGTGCCAGGGG
AGGGTCGGGCTGGATTTAGCAAGATTTACCTTCTCCAAAGAGCGGTGCTGCAGTGGCACAGCTGCCC
ACGGAGGTGGGGGGGTCACCGTCCCTGGAGGTGATGAAGAACTGTGGGGATGTGGCACTGAGGGA
CATGGCCAGTGGGCACGGTGGGTGGGTTGGGGTTGGTCTTGGGGATCTTGGAGGGCTTTTCCAGCC
TTCATGATTTGACGATTGTATGAACATCTACATGGCAATTCTCCAGCTGCCTGTCCCAGTCCTACTGAC
CCAGCTGTATCTCTCCAGGCAAGCTCTTCCACCCCTTCTGCTTGCATCCTGACACCATCAAACATGCA
GGCTCAGACACATTTCCCCGTATCCCCCCTGGTGTCTGCAGGCTCAAAGAGCAGCGAGAAGCGTTCA
GAGGAAAGCGATCCCGTGCCACCTTCCCCGTGCCCGGGCTGTCCCCGCACGCTGCCGGCTCGGGGA
TGCGGGGGGAGCGCCGGACCGGAGCGGAGCCCCGGGCGGCTCGCTGCTGCCCCCTAGCGGGGGA
GGGACGTAATTACATCCCTGGGGGCTTTGGGGGGGGGCTGTCCCCGTACAGG
Modified HS4-650 insulator (Genbank Acc. No. MN044710.1) (r-c)-A to T mutation at SA1
and SA3 (mutations in bold and underlined) (SEQ ID NO: 32)
AGCCCCATCCTCACTGACTCCGTCCTGGAGTTGGATGAGAGATAATGGCCTTACGTTGTGCCAGGGG
AGGGTCGGGCTGGATTTAGCAAGATTTACCTTCTCCAAAGAGCGGTGCTGCAGTGGCACAGCTGCCC
ACGGAGGTGGGGGGGTCACCGTCCCTGGAGGTGATGAAGAACTGTGGGGATGTGGCACTGAGGGA
CATGGCCAGTGGGCACGGTGGGTGGGTTGGGGTTGGTCTTGGGGATCTTGGAGGGCTTTTCCAGCC
TTCATGATTTGACGATTGTATGAACATCTACATGGCAATTCTCCAGCTGCCTGTCCCAGTCCTACTGAC
CCAGCTGTATCTCTCCAGGCAAGCTCTTCCACCCCTTCTGCTTGCATCCTGACACCATCAAACATGCA
GGCTCAGACACATTTCCCCGTATCCCCCCAGGTGTCTGCTGGCTCAAAGAGCAGCGAGAAGCGTTCA
GAGGAAAGCGATCCCGTGCCACCTTCCCCGTGCCCGGGCTGTCCCCGCACGCTGCCGGCTCGGGGA
TGCGGGGGGAGCGCCGGACCGGAGCGGAGCCCCGGGCGGCTCGCTGCTGCCCCCTAGCGGGGGA
GGGACGTAATTACATCCCTGGGGGCTTTGGGGGGGGGCTGTCCCCGTACAGG
Modified HS4-650 insulator (Genbank Acc. No. MN044710.1) (r-c)-A to T mutation at SA1,
SA2 and SA3 (mutations in bold and underlined) (SEQ ID NO: 33)
AGCCCCATCCTCACTGACTCCGTCCTGGAGTTGGATGAGAGATAATGGCCTTACGTTGTGCCAGGGG
AGGGTCGGGCTGGATTTAGCAAGATTTACCTTCTCCAAAGAGCGGTGCTGCAGTGGCACAGCTGCCC
ACGGAGGTGGGGGGGTCACCGTCCCTGGAGGTGATGAAGAACTGTGGGGATGTGGCACTGAGGGA
CATGGCCAGTGGGCACGGTGGGTGGGTTGGGGTTGGTCTTGGGGATCTTGGAGGGCTTTTCCAGCC
TTCATGATTTGACGATTGTATGAACATCTACATGGCAATTCTCCAGCTGCCTGTCCCAGTCCTACTGAC
CCAGCTGTATCTCTCCAGGCAAGCTCTTCCACCCCTTCTGCTTGCATCCTGACACCATCAAACATGCA
GGCTCAGACACATTTCCCCGTATCCCCCCTGGTGTCTGCTGGCTCAAAGAGCAGCGAGAAGCGTTCA
GAGGAAAGCGATCCCGTGCCACCTTCCCCGTGCCCGGGCTGTCCCCGCACGCTGCCGGCTCGGGGA
TGCGGGGGGAGCGCCGGACCGGAGCGGAGCCCCGGGCGGCTCGCTGCTGCCCCCTAGCGGGGGA
GGGACGTAATTACATCCCTGGGGGCTTTGGGGGGGGGCTGTCCCCGTACAGG
Modified HS4-650 insulator (Genbank Acc. No. MNO44710.1) (r-c)-A to T mutation at SA2
(mutation in bold and underlined) (SEQ ID NO: 34)
AGCCCCATCCTCACTGACTCCGTCCTGGAGTTGGATGAGAGATAATGGCCTTACGTTGTGCCAGGGG
AGGGTCGGGCTGGATTTAGCAAGATTTACCTTCTCCAAAGAGCGGTGCTGCAGTGGCACAGCTGCCC
ACGGAGGTGGGGGGGTCACCGTCCCTGGAGGTGATGAAGAACTGTGGGGATGTGGCACTGAGGGA
CATGGCCAGTGGGCACGGTGGGTGGGTTGGGGTTGGTCTTGGGGATCTTGGAGGGCTTTTCCAGCC
TTCATGATTTGACGATTGTATGAACATCTACATGGCAATTCTCCAGCTGCCTGTCCCAGTCCTACTGAC
CCAGCTGTATCTCTCCAGGCAAGCTCTTCCACCCCTTCTGCTTGCATCCAGACACCATCAAACATGCA
GGCTCAGACACATTTCCCCGTATCCCCCCTGGTGTCTGCAGGCTCAAAGAGCAGCGAGAAGCGTTCA
GAGGAAAGCGATCCCGTGCCACCTTCCCCGTGCCCGGGCTGTCCCCGCACGCTGCCGGCTCGGGGA
TGCGGGGGGAGCGCCGGACCGGAGCGGAGCCCCGGGCGGCTCGCTGCTGCCCCCTAGCGGGGGA
GGGACGTAATTACATCCCTGGGGGCTTTGGGGGGGGGCTGTCCCCGTACAGG
Modified HS4-650 insulator (Genbank Acc. No. MN044710.1) (r-c)-A to T mutation at SA2
and SA3 (mutations in bold and underlined) (SEQ ID NO: 35)
AGCCCCATCCTCACTGACTCCGTCCTGGAGTTGGATGAGAGATAATGGCCTTACGTTGTGCCAGGGG
AGGGTCGGGCTGGATTTAGCAAGATTTACCTTCTCCAAAGAGCGGTGCTGCAGTGGCACAGCTGCCC
ACGGAGGTGGGGGGGTCACCGTCCCTGGAGGTGATGAAGAACTGTGGGGATGTGGCACTGAGGGA
CATGGCCAGTGGGCACGGTGGGTGGGTTGGGGTTGGTCTTGGGGATCTTGGAGGGCTTTTCCAGCC
TTCATGATTTGACGATTGTATGAACATCTACATGGCAATTCTCCAGCTGCCTGTCCCAGTCCTACTGAC
CCAGCTGTATCTCTCCAGGCAAGCTCTTCCACCCCTTCTGCTTGCATCCAGACACCATCAAACATGCA
GGCTCAGACACATTTCCCCGTATCCCCCCTGGTGTCTGCTGGCTCAAAGAGCAGCGAGAAGCGTTCA
GAGGAAAGCGATCCCGTGCCACCTTCCCCGTGCCCGGGCTGTCCCCGCACGCTGCCGGCTCGGGGA
TGCGGGGGGAGCGCCGGACCGGAGCGGAGCCCCGGGCGGCTCGCTGCTGCCCCCTAGCGGGGGA
GGGACGTAATTACATCCCTGGGGGCTTTGGGGGGGGGCTGTCCCCGTACAGG
Modified HS4-650 insulator (Genbank Acc. No. MN044710.1) (r-c)-A to T mutation at SA3
(mutation in bold and underlined) (SEQ ID NO: 36)
AGCCCCATCCTCACTGACTCCGTCCTGGAGTTGGATGAGAGATAATGGCCTTACGTTGTGCCAGGGG
AGGGTCGGGCTGGATTTAGCAAGATTTACCTTCTCCAAAGAGCGGTGCTGCAGTGGCACAGCTGCCC
ACGGAGGTGGGGGGGTCACCGTCCCTGGAGGTGATGAAGAACTGTGGGGATGTGGCACTGAGGGA
CATGGCCAGTGGGCACGGTGGGTGGGTTGGGGTTGGTCTTGGGGATCTTGGAGGGCTTTTCCAGCC
TTCATGATTTGACGATTGTATGAACATCTACATGGCAATTCTCCAGCTGCCTGTCCCAGTCCTACTGAC
CCAGCTGTATCTCTCCAGGCAAGCTCTTCCACCCCTTCTGCTTGCATCCAGACACCATCAAACATGCA
GGCTCAGACACATTTCCCCGTATCCCCCCAGGTGTCTGCTGGCTCAAAGAGCAGCGAGAAGCGTTCA
GAGGAAAGCGATCCCGTGCCACCTTCCCCGTGCCCGGGCTGTCCCCGCACGCTGCCGGCTCGGGGA
TGCGGGGGGAGCGCCGGACCGGAGCGGAGCCCCGGGCGGCTCGCTGCTGCCCCCTAGCGGGGGA
GGGACGTAATTACATCCCTGGGGGCTTTGGGGGGGGGCTGTCCCCGTACAGG
Unmodified HS4-650 insulator (Genbank Acc. No. MN044709) (SEQ ID NO: 37)
CCTGTACGGGGACAGCCCCCCCCCAAAGCCCCCAGGGATGTAATTACGTCCCTCCCCCGCTAGGGG
GCAGCAGCGAGCCGCCCGGGGCTCCGCTCCGGTCCGGCGCTCCCCCCGCATCCCCGAGCCGGCAG
CGTGCGGGGACAGCCCGGGCACGGGGAAGGTGGCACGGGATCGCTTTCCTCTGAACGCTTCTCGCT
GCTCTTTGAGCCTGCAGACACCTGGGGGGATACGGGGAAATGTGTCTGAGCCTGCATGTTTGATGGT
GTCTGGATGCAAGCAGAAGGGGTGGAAGAGCTTGCCTGGAGAGATACAGCTGGGTCAGTAGGACTG
GGACAGGCAGCTGGAGAATTGCCATGTAGATGTTCATACAATCGTCAAATCATGAAGGCTGGAAAAG
CCCTCCAAGATCCCCAAGACCAACCCCAACCCACCCACCGTGCCCACTGGCCATGTCCCTCAGTGCC
ACATCCCCACAGTTCTTCATCACCTCCAGGGACGGTGACCCCCCCACCTCCGTGGGCAGCTGTGCCA
CTGCAGCACCGCTCTTTGGAGAAGGTAAATCTTGCTAAATCCAGCCCGACCCTCCCCTGGCACAACGT
AAGGCCATTATCTCTCATCCAACTCCAGGACGGAGTCAGTGAGGATGGGGCT
Reverse complement of unmodified HS4-650 insulator (Genbank Acc. No. MN044709) (SEQ
ID NO: 38)
AGCCCCATCCTCACTGACTCCGTCCTGGAGTTGGATGAGAGATAATGGCCTTACGTTGTGCCAGGGG
AGGGTCGGGCTGGATTTAGCAAGATTTACCTTCTCCAAAGAGCGGTGCTGCAGTGGCACAGCTGCCC
ACGGAGGTGGGGGGGTCACCGTCCCTGGAGGTGATGAAGAACTGTGGGGATGTGGCACTGAGGGA
CATGGCCAGTGGGCACGGTGGGGGGTTGGGGTTGGTCTTGGGGATCTTGGAGGGCTTTTCCAGCC
TTCATGATTTGACGATTGTATGAACATCTACATGGCAATTCTCCAGCTGCCTGTCCCAGTCCTACTGAC
CCAGCTGTATCTCTCCAGGCAAGCTCTTCCACCCCTTCTGCTTGCATCCAGACACCATCAAACATGCA
GGCTCAGACACATTTCCCCGTATCCCCCCAGGTGTCTGCAGGCTCAAAGAGCAGCGAGAAGCGTTCA
GAGGAAAGCGATCCCGTGCCACCTTCCCCGTGCCCGGGCTGTCCCCGCACGCTGCCGGCTCGGGGA
TGCGGGGGGAGCGCCGGACCGGAGCGGAGCCCCGGGCGGCTCGCTGCTGCCCCCTAGCGGGGGA
GGGACGTAATTACATCCCTGGGGGCTTTGGGGGGGGGCTGTCCCCGTG
Modified HS4-650 insulator (Genbank Acc. No. MN044709) (r-c)-A to T mutation at SA1
(mutation in bold and underlined) (SEQ ID NO: 39)
AGCCCCATCCTCACTGACTCCGTCCTGGAGTTGGATGAGAGATAATGGCCTTACGTTGTGCCAGGGG
AGGGTCGGGCTGGATTTAGCAAGATTTACCTTCTCCAAAGAGCGGTGCTGCAGTGGCACAGCTGCCC
ACGGAGGTGGGGGGGTCACCGTCCCTGGAGGTGATGAAGAACTGTGGGGATGTGGCACTGAGGGA
CATGGCCAGTGGGCACGGTGGGTGGGTTGGGGTTGGTCTTGGGGATCTTGGAGGGCTTTTCCAGCC
TTCATGATTTGACGATTGTATGAACATCTACATGGCAATTCTCCAGCTGCCTGTCCCAGTCCTACTGAC
CCAGCTGTATCTCTCCAGGCAAGCTCTTCCACCCCTTCTGCTTGCATCCTGACACCATCAAACATGCA
GGCTCAGACACATTTCCCCGTATCCCCCCAGGTGTCTGCAGGCTCAAAGAGCAGCGAGAAGCGTTCA
GAGGAAAGCGATCCCGTGCCACCTTCCCCGTGCCCGGGCTGTCCCCGCACGCTGCCGGCTCGGGGA
TGCGGGGGGAGCGCCGGACCGGAGCGGAGCCCCGGGCGGCTCGCTGCTGCCCCCTAGCGGGGGA
GGGACGTAATTACATCCCTGGGGGCTTTGGGGGGGGGCTGTCCCCGTG
Modified HS4-650 insulator (Genbank Acc. No. MN044709) (r-c)-A to T mutation at SA1
and SA2 (mutations in bold and underlined) (SEQ ID NO: 40)
AGCCCCATCCTCACTGACTCCGTCCTGGAGTTGGATGAGAGATAATGGCCTTACGTTGTGCCAGGGG
AGGGTCGGGCTGGATTTAGCAAGATTTACCTTCTCCAAAGAGCGGTGCTGCAGTGGCACAGCTGCCC
ACGGAGGTGGGGGGGTCACCGTCCCTGGAGGTGATGAAGAACTGTGGGGATGTGGCACTGAGGGA
CATGGCCAGTGGGCACGGTGGGTGGGTTGGGGTTGGTCTTGGGGATCTTGGAGGGCTTTTCCAGCC
TTCATGATTTGACGATTGTATGAACATCTACATGGCAATTCTCCAGCTGCCTGTCCCAGTCCTACTGAC
CCAGCTGTATCTCTCCAGGCAAGCTCTTCCACCCCTTCTGCTTGCATCCTGACACCATCAAACATGCA
GGCTCAGACACATTTCCCCGTATCCCCCCTGGTGTCTGCAGGCTCAAAGAGCAGCGAGAAGCGTTCA
GAGGAAAGCGATCCCGTGCCACCTTCCCCGTGCCCGGGCTGTCCCCGCACGCTGCCGGCTCGGGGA
TGCGGGGGGAGCGCCGGACCGGAGCGGAGCCCCGGGCGGCTCGCTGCTGCCCCCTAGCGGGGGA
GGGACGTAATTACATCCCTGGGGGCTTTGGGGGGGGGCTGTCCCCGTG
Modified HS4-650 insulator (Genbank Acc. No. MNO44709) (r-c)-A to T mutation at SA1
and SA3 (mutations in bold and underlined) (SEQ ID NO: 41)
AGCCCCATCCTCACTGACTCCGTCCTGGAGTTGGATGAGAGATAATGGCCTTACGTTGTGCCAGGGG
AGGGTCGGGCTGGATTTAGCAAGATTTACCTTCTCCAAAGAGCGGTGCTGCAGTGGCACAGCTGCCC
ACGGAGGTGGGGGGGTCACCGTCCCTGGAGGTGATGAAGAACTGTGGGGATGTGGCACTGAGGGA
CATGGCCAGTGGGCACGGTGGGTGGGTTGGGGTTGGTCTTGGGGATCTTGGAGGGCTTTTCCAGCC
TTCATGATTTGACGATTGTATGAACATCTACATGGCAATTCTCCAGCTGCCTGTCCCAGTCCTACTGAC
CCAGCTGTATCTCTCCAGGCAAGCTCTTCCACCCCTTCTGCTTGCATCCTGACACCATCAAACATGCA
GGCTCAGACACATTTCCCCGTATCCCCCCAGGTGTCTGCTGGCTCAAAGAGCAGCGAGAAGCGTTCA
GAGGAAAGCGATCCCGTGCCACCTTCCCCGTGCCCGGGCTGTCCCCGCACGCTGCCGGCTCGGGGA
TGCGGGGGGAGCGCCGGACCGGAGCGGAGCCCCGGGCGGCTCGCTGCTGCCCCCTAGCGGGGGA
GGGACGTAATTACATCCCTGGGGGCTTTGGGGGGGGGCTGTCCCCGTG
Modified HS4-650 insulator (Genbank Acc. No. MN044709) (r-c)-A to T mutation at SA1,
SA2 and SA3 (mutations in bold and underlined) (SEQ ID NO: 42)
AGCCCCATCCTCACTGACTCCGTCCTGGAGTTGGATGAGAGATAATGGCCTTACGTTGTGCCAGGGG
AGGGTCGGGCTGGATTTAGCAAGATTTACCTTCTCCAAAGAGCGGTGCTGCAGTGGCACAGCTGCCC
ACGGAGGTGGGGGGGTCACCGTCCCTGGAGGTGATGAAGAACTGTGGGGATGTGGCACTGAGGGA
CATGGCCAGTGGGCACGGTGGGTGGGTTGGGGTTGGTCTTGGGGATCTTGGAGGGCTTTTCCAGCC
TTCATGATTTGACGATTGTATGAACATCTACATGGCAATTCTCCAGCTGCCTGTCCCAGTCCTACTGAC
CCAGCTGTATCTCTCCAGGCAAGCTCTTCCACCCCTTCTGCTTGCATCCTGACACCATCAAACATGCA
GGCTCAGACACATTTCCCCGTATCCCCCCTGGTGTCTGCTGGCTCAAAGAGCAGCGAGAAGCGTTCA
GAGGAAAGCGATCCCGTGCCACCTTCCCCGTGCCCGGGCTGTCCCCGCACGCTGCCGGCTCGGGGA
TGCGGGGGGAGCGCCGGACCGGAGCGGAGCCCCGGGCGGCTCGCTGCTGCCCCCTAGCGGGGGA
GGGACGTAATTACATCCCTGGGGGCTTTGGGGGGGGGCTGTCCCCGTG
Modified HS4-650 insulator (Genbank Acc. No. MN044709) (r-c)-A to T mutation at SA2
(mutation in bold and underlined) (SEQ ID NO: 43)
AGCCCCATCCTCACTGACTCCGTCCTGGAGTTGGATGAGAGATAATGGCCTTACGTTGTGCCAGGGG
AGGGTCGGGCTGGATTTAGCAAGATTTACCTTCTCCAAAGAGCGGTGCTGCAGTGGCACAGCTGCCC
ACGGAGGTGGGGGGGTCACCGTCCCTGGAGGTGATGAAGAACTGTGGGGATGTGGCACTGAGGGA
CATGGCCAGTGGGCACGGTGGGTGGGTTGGGGTTGGTCTTGGGGATCTTGGAGGGCTTTTCCAGCC
TTCATGATTTGACGATTGTATGAACATCTACATGGCAATTCTCCAGCTGCCTGTCCCAGTCCTACTGAC
CCAGCTGTATCTCTCCAGGCAAGCTCTTCCACCCCTTCTGCTTGCATCCAGACACCATCAAACATGCA
GGCTCAGACACATTTCCCCGTATCCCCCCTGGTGTCTGCAGGCTCAAAGAGCAGCGAGAAGCGTTCA
GAGGAAAGCGATCCCGTGCCACCTTCCCCGTGCCCGGGCTGTCCCCGCACGCTGCCGGCTCGGGGA
TGCGGGGGGAGCGCCGGACCGGAGCGGAGCCCCGGGCGGCTCGCTGCTGCCCCCTAGCGGGGGA
GGGACGTAATTACATCCCTGGGGGCTTTGGGGGGGGGCTGTCCCCGTG
Modified HS4-650 insulator (Genbank Acc. No. MN044709) (r-c)-A to T mutation at SA2
and SA3 (mutations in bold and underlined) (SEQ ID NO: 44)
AGCCCCATCCTCACTGACTCCGTCCTGGAGTTGGATGAGAGATAATGGCCTTACGTTGTGCCAGGGG
AGGGTCGGGCTGGATTTAGCAAGATTTACCTTCTCCAAAGAGCGGTGCTGCAGTGGCACAGCTGCCC
ACGGAGGTGGGGGGGTCACCGTCCCTGGAGGTGATGAAGAACTGTGGGGATGTGGCACTGAGGGA
CATGGCCAGTGGGCACGGTGGGTGGGTTGGGGTTGGTCTTGGGGATCTTGGAGGGCTTTTCCAGCC
TTCATGATTTGACGATTGTATGAACATCTACATGGCAATTCTCCAGCTGCCTGTCCCAGTCCTACTGAC
CCAGCTGTATCTCTCCAGGCAAGCTCTTCCACCCCTTCTGCTTGCATCCAGACACCATCAAACATGCA
GGCTCAGACACATTTCCCCGTATCCCCCCTGGTGTCTGCTGGCTCAAAGAGCAGCGAGAAGCGTTCA
GAGGAAAGCGATCCCGTGCCACCTTCCCCGTGCCCGGGCTGTCCCCGCACGCTGCCGGCTCGGGGA
TGCGGGGGGAGCGCCGGACCGGAGCGGAGCCCCGGGCGGCTCGCTGCTGCCCCCTAGCGGGGGA
GGGACGTAATTACATCCCTGGGGGCTTTGGGGGGGGGCTGTCCCCGTG
Modified HS4-650 insulator (Genbank Acc. No. MN044709) (r-c)-A to T mutation at SA3
(mutation in bold and underlined) (SEQ ID NO: 45)
AGCCCCATCCTCACTGACTCCGTCCTGGAGTTGGATGAGAGATAATGGCCTTACGTTGTGCCAGGGG
AGGGTCGGGCTGGATTTAGCAAGATTTACCTTCTCCAAAGAGCGGTGCTGCAGTGGCACAGCTGCCC
ACGGAGGTGGGGGGGTCACCGTCCCTGGAGGTGATGAAGAACTGTGGGGATGTGGCACTGAGGGA
CATGGCCAGTGGGCACGGTGGGTGGGTTGGGGTTGGTCTTGGGGATCTTGGAGGGCTTTTCCAGCC
TTCATGATTTGACGATTGTATGAACATCTACATGGCAATTCTCCAGCTGCCTGTCCCAGTCCTACTGAC
CCAGCTGTATCTCTCCAGGCAAGCTCTTCCACCCCTTCTGCTTGCATCCAGACACCATCAAACATGCA
GGCTCAGACACATTTCCCCGTATCCCCCCAGGTGTCTGCTGGCTCAAAGAGCAGCGAGAAGCGTTCA
GAGGAAAGCGATCCCGTGCCACCTTCCCCGTGCCCGGGCTGTCCCCGCACGCTGCCGGCTCGGGGA
TGCGGGGGGAGCGCCGGACCGGAGCGGAGCCCCGGGCGGCTCGCTGCTGCCCCCTAGCGGGGGA
GGGACGTAATTACATCCCTGGGGGCTTTGGGGGGGGGCTGTCCCCGTG
Unmodified HS4-650 insulator (Genbank Acc. No. KF569217) (SEQ ID NO: 46)
CGGGGACAGCCCCCCCCCAAAGCCCCCAGGGATGTAATTACGTCCCTCCCCCGCTAGGGGGCAGCA
GCGAGCCGCCCGGGGCTCCGCTCCGGTCCGGCGCTCCCCCCGCATCCCCGAGCCGGCAGCGTGCG
GGGACAGCCCGGGCACGGGGAAGGTGGCACGGGATCGCTTTCCTCTGAACGCTTCTCGCTGCTCTTT
GAGCCTGCAGACACCTGGGGGGATACGGGGAAAATGTGTCTGAGCCTGCATGTTTGATGGTGTCTG
GATGCAAGCAGAAGGGGTGGAAGAGCTTGCCTGGAGAGATACAGCTGGGTCAGTAGGACTGGGACA
GGCAGCTGGAGAATTGCCATGTAGATGTTCATACAATCGTCAAATCATGAAGGCTGGAAAAGCCCTC
CAAGATCCCCAAGACCAACCCCAACCCACCCACCGTGCCCACTGGCCATGTCCCTCAGTGCCACATC
CCCACAGTTCTTCATCACCTCCAGGGACGGTGACCCCCCCACCTCCGTGGGCAGCTGTGCCACTGCA
GCACCGCTCTTTGGAGAAGGTAAATCTTGCTAAATCCAGCCCGACCCTCCCCTGGCACAACGTAAGG
CCATTATCTCTCATCCAACTCCAGGACGGAGTCAGTGAGAATA
Reverse complement of unmodified HS4-650 insulator (Genbank Acc. No. KF569217) (SEQ
ID NO: 47)
TATTCTCACTGACTCCGTCCTGGAGTTGGATGAGAGATAATGGCCTTACGTTGTGCCAGGGGAGGGT
CGGGCTGGATTTAGCAAGATTTACCTTCTCCAAAGAGCGGTGCTGCAGTGGCACAGCTGCCCACGGA
GGTGGGGGGGTCACCGTCCCTGGAGGTGATGAAGAACTGTGGGGATGTGGCACTGAGGGACATGG
CCAGTGGGCACGGTGGGTGGGTTGGGGTTGGTCTTGGGGATCTTGGAGGGCTTTTCCAGCCTTCAT
GATTTGACGATTGTATGAACATCTACATGGCAATTCTCCAGCTGCCTGTCCCAGTCCTACTGACCCAG
CTGTATCTCTCCAGGCAAGCTCTTCCACCCCTTCTGCTTGCATCCAGACACCATCAAACATGCAGGCT
CAGACACATTTTCCCCGTATCCCCCCAGGTGTCTGCAGGCTCAAAGAGCAGCGAGAAGCGTTCAGAG
GAAAGCGATCCCGTGCCACCTTCCCCGTGCCCGGGCTGTCCCCGCACGCTGCCGGCTCGGGGATGC
GGGGGGAGCGCCGGACCGGAGCGGAGCCCCGGGCGGCTCGCTGCTGCCCCCTAGCGGGGGAGGG
ACGTAATTACATCCCTGGGGGCTTTGGGGGGGGGCTGTCCCCG
Modified HS4-650 insulator (Genbank Acc. No. KF569217) (r-c)-A to T mutation at SA1
(mutation in bold and underlined) (SEQ ID NO: 48)
TATTCTCACTGACTCCGTCCTGGAGTTGGATGAGAGATAATGGCCTTACGTTGTGCCAGGGGAGGGT
CGGGCTGGATTTAGCAAGATTTACCTTCTCCAAAGAGCGGTGCTGCAGTGGCACAGCTGCCCACGGA
GGTGGGGGGGTCACCGTCCCTGGAGGTGATGAAGAACTGTGGGGATGTGGCACTGAGGGACATGG
CCAGTGGGCACGGTGGGTGGGTTGGGGTTGGTCTTGGGGATCTTGGAGGGCTTTTCCAGCCTTCAT
GATTTGACGATTGTATGAACATCTACATGGCAATTCTCCAGCTGCCTGTCCCAGTCCTACTGACCCAG
CTGTATCTCTCCAGGCAAGCTCTTCCACCCCTTCTGCTTGCATCCTGACACCATCAAACATGCAGGCT
CAGACACATTTTCCCCGTATCCCCCCAGGTGTCTGCAGGCTCAAAGAGCAGCGAGAAGCGTTCAGAG
GAAAGCGATCCCGTGCCACCTTCCCCGTGCCCGGGCTGTCCCCGCACGCTGCCGGCTCGGGGATGC
GGGGGGAGCGCCGGACCGGAGCGGAGCCCCGGGCGGCTCGCTGCTGCCCCCTAGCGGGGGAGGG
ACGTAATTACATCCCTGGGGGCTTTGGGGGGGGGCTGTCCCCG
Modified HS4-650 insulator (Genbank Acc. No. KF569217) (r-c)-A to T mutation at SA1 and
SA2 (mutation in bold and underlined) (SEQ ID NO: 49)
TATTCTCACTGACTCCGTCCTGGAGTTGGATGAGAGATAATGGCCTTACGTTGTGCCAGGGGAGGGT
CGGGCTGGATTTAGCAAGATTTACCTTCTCCAAAGAGCGGTGCTGCAGTGGCACAGCTGCCCACGGA
GGTGGGGGGGTCACCGTCCCTGGAGGTGATGAAGAACTGTGGGGATGTGGCACTGAGGGACATGG
CCAGTGGGCACGGTGGGTGGGTTGGGGTTGGTCTTGGGGATCTTGGAGGGCTTTTCCAGCCTTCAT
GATTTGACGATTGTATGAACATCTACATGGCAATTCTCCAGCTGCCTGTCCCAGTCCTACTGACCCAG
CTGTATCTCTCCAGGCAAGCTCTTCCACCCCTTCTGCTTGCATCCTGACACCATCAAACATGCAGGCT
CAGACACATTTTCCCCGTATCCCCCCTGGTGTCTGCAGGCTCAAAGAGCAGCGAGAAGCGTTCAGAG
GAAAGCGATCCCGTGCCACCTTCCCCGTGCCCGGGCTGTCCCCGCACGCTGCCGGCTCGGGGATGC
GGGGGGAGCGCCGGACCGGAGCGGAGCCCCGGGCGGCTCGCTGCTGCCCCCTAGCGGGGGAGGG
ACGTAATTACATCCCTGGGGGCTTTGGGGGGGGGCTGTCCCCG
Modified HS4-650 insulator (Genbank Acc. No. KF569217) (r-c)-A to T mutation at SA1 and
SA3 (mutation in bold and underlined) (SEQ ID NO: 50)
TATTCTCACTGACTCCGTCCTGGAGTTGGATGAGAGATAATGGCCTTACGTTGTGCCAGGGGAGGGT
CGGGCTGGATTTAGCAAGATTTACCTTCTCCAAAGAGCGGTGCTGCAGTGGCACAGCTGCCCACGGA
GGTGGGGGGGTCACCGTCCCTGGAGGTGATGAAGAACTGTGGGGATGTGGCACTGAGGGACATGG
CCAGTGGGCACGGTGGGTGGGTTGGGGTTGGTCTTGGGGATCTTGGAGGGCTTTTCCAGCCTTCAT
GATTTGACGATTGTATGAACATCTACATGGCAATTCTCCAGCTGCCTGTCCCAGTCCTACTGACCCAG
CTGTATCTCTCCAGGCAAGCTCTTCCACCCCTTCTGCTTGCATCCTGACACCATCAAACATGCAGGCT
CAGACACATTTTCCCCGTATCCCCCCAGGTGTCTGCTGGCTCAAAGAGCAGCGAGAAGCGTTCAGAG
GAAAGCGATCCCGTGCCACCTTCCCCGTGCCCGGGCTGTCCCCGCACGCTGCCGGCTCGGGGATGC
GGGGGGAGCGCCGGACCGGAGCGGAGCCCCGGGCGGCTCGCTGCTGCCCCCTAGCGGGGGAGGG
ACGTAATTACATCCCTGGGGGCTTTGGGGGGGGGCTGTCCCCG
Modified HS4-650 insulator (Genbank Acc. No. KF569217) (r-c)-A to T mutation at SA1,
SA2 and SA3 (mutation in bold and underlined) (SEQ ID NO: 51)
TATTCTCACTGACTCCGTCCTGGAGTTGGATGAGAGATAATGGCCTTACGTTGTGCCAGGGGAGGGT
CGGGCTGGATTTAGCAAGATTTACCTTCTCCAAAGAGCGGTGCTGCAGTGGCACAGCTGCCCACGGA
GGTGGGGGGGTCACCGTCCCTGGAGGTGATGAAGAACTGTGGGGATGTGGCACTGAGGGACATGG
CCAGTGGGCACGGTGGGTGGGTTGGGGTTGGTCTTGGGGATCTTGGAGGGCTTTTCCAGCCTTCAT
GATTTGACGATTGTATGAACATCTACATGGCAATTCTCCAGCTGCCTGTCCCAGTCCTACTGACCCAG
CTGTATCTCTCCAGGCAAGCTCTTCCACCCCTTCTGCTTGCATCCTGACACCATCAAACATGCAGGCT
CAGACACATTTTCCCCGTATCCCCCCTGGTGTCTGCTGGCTCAAAGAGCAGCGAGAAGCGTTCAGAG
GAAAGCGATCCCGTGCCACCTTCCCCGTGCCCGGGCTGTCCCCGCACGCTGCCGGCTCGGGGATGC
GGGGGGAGCGCCGGACCGGAGCGGAGCCCCGGGCGGCTCGCTGCTGCCCCCTAGCGGGGGAGGG
ACGTAATTACATCCCTGGGGGCTTTGGGGGGGGGCTGTCCCCG
Modified HS4-650 insulator (Genbank Acc. No. KF569217) (r-c)-A to T mutation at SA2
(mutation in bold and underlined) (SEQ ID NO: 52)
TATTCTCACTGACTCCGTCCTGGAGTTGGATGAGAGATAATGGCCTTACGTTGTGCCAGGGGAGGGT
CGGGCTGGATTTAGCAAGATTTACCTTCTCCAAAGAGCGGTGCTGCAGTGGCACAGCTGCCCACGGA
GGTGGGGGGGTCACCGTCCCTGGAGGTGATGAAGAACTGTGGGGATGTGGCACTGAGGGACATGG
CCAGTGGGCACGGTGGGTGGGTTGGGGTTGGTCTTGGGGATCTTGGAGGGCTTTTCCAGCCTTCAT
GATTTGACGATTGTATGAACATCTACATGGCAATTCTCCAGCTGCCTGTCCCAGTCCTACTGACCCAG
CTGTATCTCTCCAGGCAAGCTCTTCCACCCCTTCTGCTTGCATCCAGACACCATCAAACATGCAGGCT
CAGACACATTTTCCCCGTATCCCCCCTGGTGTCTGCAGGCTCAAAGAGCAGCGAGAAGCGTTCAGAG
GAAAGCGATCCCGTGCCACCTTCCCCGTGCCCGGGCTGTCCCCGCACGCTGCCGGCTCGGGGATGC
GGGGGGAGCGCCGGACCGGAGCGGAGCCCCGGGCGGCTCGCTGCTGCCCCCTAGCGGGGGAGGG
ACGTAATTACATCCCTGGGGGCTTTGGGGGGGGGCTGTCCCCG
Modified HS4-650 insulator (Genbank Acc. No. KF569217) (r-c)-A to T mutation at SA2 and
SA3 (mutation in bold and underlined) (SEQ ID NO: 53)
TATTCTCACTGACTCCGTCCTGGAGTTGGATGAGAGATAATGGCCTTACGTTGTGCCAGGGGAGGGT
CGGGCTGGATTTAGCAAGATTTACCTTCTCCAAAGAGCGGTGCTGCAGTGGCACAGCTGCCCACGGA
GGTGGGGGGGTCACCGTCCCTGGAGGTGATGAAGAACTGTGGGGATGTGGCACTGAGGGACATGG
CCAGTGGGCACGGTGGGTGGGTTGGGGTTGGTCTTGGGGATCTTGGAGGGCTTTTCCAGCCTTCAT
GATTTGACGATTGTATGAACATCTACATGGCAATTCTCCAGCTGCCTGTCCCAGTCCTACTGACCCAG
CTGTATCTCTCCAGGCAAGCTCTTCCACCCCTTCTGCTTGCATCCAGACACCATCAAACATGCAGGCT
CAGACACATTTTCCCCGTATCCCCCCTGGTGTCTGCTGGCTCAAAGAGCAGCGAGAAGCGTTCAGAG
GAAAGCGATCCCGTGCCACCTTCCCCGTGCCCGGGCTGTCCCCGCACGCTGCCGGCTCGGGGATGC
GGGGGGAGCGCCGGACCGGAGCGGAGCCCCGGGCGGCTCGCTGCTGCCCCCTAGCGGGGGAGGG
ACGTAATTACATCCCTGGGGGCTTTGGGGGGGGGCTGTCCCCG
Modified HS4-650 insulator (Genbank Acc. No. KF569217) (r-c)-A to T mutation at SA3
(mutation in bold and underlined) (SEQ ID NO: 54)
TATTCTCACTGACTCCGTCCTGGAGTTGGATGAGAGATAATGGCCTTACGTTGTGCCAGGGGAGGGT
CGGGCTGGATTTAGCAAGATTTACCTTCTCCAAAGAGCGGTGCTGCAGTGGCACAGCTGCCCACGGA
GGTGGGGGGGTCACCGTCCCTGGAGGTGATGAAGAACTGTGGGGATGTGGCACTGAGGGACATGG
CCAGTGGGCACGGTGGGTGGGTTGGGGTTGGTCTTGGGGATCTTGGAGGGCTTTTCCAGCCTTCAT
GATTTGACGATTGTATGAACATCTACATGGCAATTCTCCAGCTGCCTGTCCCAGTCCTACTGACCCAG
CTGTATCTCTCCAGGCAAGCTCTTCCACCCCTTCTGCTTGCATCCAGACACCATCAAACATGCAGGCT
CAGACACATTTTCCCCGTATCCCCCCAGGTGTCTGCTGGCTCAAAGAGCAGCGAGAAGCGTTCAGAG
GAAAGCGATCCCGTGCCACCTTCCCCGTGCCCGGGCTGTCCCCGCACGCTGCCGGCTCGGGGATGC
GGGGGGAGCGCCGGACCGGAGCGGAGCCCCGGGCGGCTCGCTGCTGCCCCCTAGCGGGGGAGGG
ACGTAATTACATCCCTGGGGGCTTTGGGGGGGGGCTGTCCCCG
pBRNGTR47 pTL20c SK734rev MND WAS 650 (SEQ ID NO: 55)
ggccgcctcggccaaacagcccttgagtttaccactccctatcagtgatagagaaaagtgaaagtcgagtttaccactccctatcagt
gatagagaaaagtgaaagtcgagtttaccactccctatcagtgatagagaaaagtgaaagtcgagtttaccactccctatcagtgat
agagaaaagtgaaagtcgagtttaccagtccctatcagtgatagagaaaagtgaaagtcgagtttaccactccctatcagtgatag
agaaaagtgaaagtcgagtttaccactccctatcagtgatagagaaaagtgaaagtcgagctcgccatgggaggcgtggcctggg
cgggactggggagtggcgagccctcagatcctgcatataagcagctgctttttgcctgtactgggtctctctggttagaccagatctg
agcctgggagctctctggctaactagggaacccactgcttaagcctcaataaagcttgccttgagtgcttcaagtagtgtgtgcccgt
ctgttgtgtgactctggtaactagagatccctcagacccttttagtcagtgtggaaaatctctagcagtggcgcccgaacagggactt
gaaagcgaaagggaaaccagaggagctctctcgacgcaggactcggcttgctgaagcgcgcacggcaagaggcgaggggcgg
cgactggtgagtacgccaaaaattttgactagcggaggctagaaggagagagatgggtgcgagagcgtcagtattaagcggggg
agaattagatcgcgatgggaaaaaattcggttaaggccagggggaaagaaaaaatataaattaaaacatatagtatgggcaagc
agggagctagaacgattcgcagttaatactggcctgttagaaacatcagaaggctgtagacaaatactgggacagctacaaccatc
ccttcagacaggatcagaagaacttagatcattatataatacagtagcaaccctctattgtgtgcatcaaaggatagagataaaaga
caccaaggaagctttagacaagatagaggaagagcaaaacaaaagtaagaaaaaagcacagcaagcagcaggatcttcagac
ctggaaattccctacaatccccaaagtcaaggagtagtagaatctatgaataaagaattaaagaaaattataggacaggtaagag
atcaggctgaacatcttaagacagcagtacaaatggcagtattcatccacaattttaaaagaaaaggggggattggggggtacagt
gcaggggaaagaatagtagacataatagcaacagacatacaaactaaagaattacaaaaacaaattacaaaaattcaaaattttc
gggtttattacagggacagcagaaatccactttggaaaggaccagcaaagctcctctggaaaggtgaaggggcagtagtaataca
agataatagtgacataaaagtagtgccaagaagaaaagcaaagatcattagggattatggaaaacagatggcaggtgatgattg
tgtggcaagtagacaggatgaggattagaacatggaaaagtttagtaaaacaccataaggaggagatatgagggacaattggag
aagtgaattatataaatataaagtagtaaaaattgaaccattaggagtagcacccaccaaggcaaagagaagagtggtgcagag
agaaaaaagagcagtgggaataggagctttgttccttgggttcttgggagcagcaggaagcactatgggcgcagcgtcaatgacg
ctgacggtacaggccagacaattattgtctggtatagtgcagcagcagaacaatttgctgagggctattgaggcgcaacagcatct
gttgcaactcacagtctggggcatcaagcagctccaggcaagaatcctggctgtggaaagatacctaaaggatcaacagctcctgg
ggatttggggttgctctggaaaactcatttgcaccactgctgtgccttggaatgctagttggagtaataaatctctggaacagatttgg
aatcacacgacctggatggagtgggacagagaaattaacaattacacaagcttaatacactccttaattgaagaatcgcaaaacca
gcaagaaaagaatgaacaagaattattggaattagataaatgggcaagtttgtggaattggtttaacataacaaattggctgtggt
atataaaattattcataatgatagtaggaggcttggtaggtttaagaatagtttttgctgtactttctatagtgaatagagttaggcag
ggatattcaccattatcgtttcagacccacctcccaaccccgaggggaccgagctcaagcttcgaagcgatcgcacgcgtcaaaaa
aggatatgcccttgactatgtcggacaaatagtcaagggcatatcctgaggtacccaggcggcgcacaagctatataaacctgaag
gaaatctcaactttacacttaggtcaagttacttatcgtactagagcttcagcaggaaatttaactaaaatctaatttaaccagcatag
caaatatcatttattcccaaaatgctaaagtttgagataaacggacttgatttccggctgttttgacactatccagaatgccttgcagat
gggtggggcatgctaaatactgcacgtcgatacgcgtggatccgaacagagagacagcagaatatgggccaaacaggatatctgt
ggtaagcagttcctgccccggctcagggccaagaacagttggaacagcagaatatgggccaaacaggatatctgtggtaagcagt
tcctgccccggctcagggccaagaacagatggtccccagatgcggtcccgccctcagcagtttctagagaaccatcagatgtttcca
gggtgccccaaggacctgaaatgaccctgtgccttatttgaactaaccaatcagttcgcttctcgcttctgttcgcgcgcttctgctcccc
gagctctatataagcagagctcgtttagtgaaccgtcagatcggcgcgccaattcaagcgagaagacaagggcagccgccaccat
gagtgggggcccaatgggaggaaggcccgggggccgaggagcaccagcggttcagcagaacataccctccaccctcctccagg
accacgagaaccagcgactctttgagatgcttggacgaaaatgcttgacgctggccactgcagttgttcagctgtacctggcgctgc
cccctggagctgagcactggaccaaggagcattgtggggctgtgtgcttcgtgaaggataacccccagaagtcctacttcatccgcc
tttacggccttcaggctggtcggctgctctgggaacaggagctgtactcacagcttgtctactccacccccacccccttcttccacacct
tcgctggagatgactgccaagcggggctgaactttgcagacgaggacgaggcccaggccttccgggcactcgtgcaggagaaga
tacaaaaaaggaatcagaggcaaagtggagacagacgccagctacccccaccaccaacaccagccaatgaagagagaagagg
agggctcccacccctgcccctgcatccaggtggagaccaaggaggccctccagtgggtccgctctccctggggctggcgacagtgg
acatccagaaccctgacatcacgagttcacgataccgtgggctcccagcacctggacctagcccagctgataagaaacgctcaggg
aagaagaagatcagcaaagctgatattggtgcacccagtggattcaagcatgtcagccacgtggggtgggacccccagaatggat
ttgacgtgaacaacctcgacccagatctgcggagtctgttctccagggcaggaatcagcgaggcccagctcaccgacgccgagacc
tctaaacttatctacgacttcattgaggaccagggtgggctggaggctgtgcggcaggagatgaggcgccaggagccacttccgcc
gcccccaccgccatctcgaggagggaaccagctcccccggccccctattgtggggggtaacaagggtcgttctggtccactgccccc
tgtacctttggggattgccccacccccaccaacaccccggggacccccacccccaggccgagggggtcctccaccaccaccccctcc
agctactggacgttctggaccactgccccctccaccccctggagctggtgggccacccatgccaccaccaccgccaccaccgccacc
gccgcccagctccgggaatggaccagcccctcccccactccctcctgctctggtgcctgccgggggcctggcccctggtgggggtcg
gggagcgcttttggatcaaatccggcagggaattcagctgaacaagacccctggggccccagagagctcagcgctgcagccacca
cctcagagctcagagggactggtgggggccctgatgcacgtgatgcagaagagaagcagagccatccactcctccgacgaaggg
gaggaccaggctggcgatgaagatgaagatgatgaatgggatgactgataactagtaatcaacctctggattacaaaatttgtgaa
agattgactggtattcttaactatgttgctccttttacgctatgtggatacgctgctttaatgcctttgtatcatgctattgcttcccgtatg
gctttcattttctcctccttgtataaatcctggttgctgtctctttatgaggagttgtggcccgttgtcaggcaacgtggcgtggtgtgcac
tgtgtttgctgacgcaacccccactggttggggcattgccaccacctgtcagctcctttccgggactttcgctttccccctccctattgcc
acggcggaactcatcgccgcctgccttgcccgctgctggacaggggctcggctgttgggcactgacaattccgtggtgttgtcgggg
aaatcatcgtcctttccttggctgttcgcctgtgttgccacctggattctgcgcgggacgtccttctgctacgtcccttcggccctcaatcc
agcggaccttccttcccgcggcctgctgccggctctgcggcctcttccgcgtcttcgccttcgccctcagacgagtcggatctccctttg
ggccgcctccccgcacgtacgaccggtgcggccgcatcgatgccgtagtacctttaagaccaatgacttacaaggcagctgtagatc
ttagccactttttaaaagaaaaggggggactggaagggctaattcactcccaaagaagacaagagccccatcctcactgactccgt
cctggagttggatgagagataatggccttacgttgtgccaggggagggtcgggctggatttagcaagatttaccttctccaaagagc
ggtgctgcagtggcacagctgcccacggaggtgggggggtcaccgtccctggaggtgatgaagaactgtggggatgtggcactg
agggacatggccagtgggcacggtgggtgggttggggttggtcttggggatcttggagggcttttccagccttcatgatttgacgatt
gtatgaacatctacatggcaattctccagctgcctgtcccagtcctactgacccagctgtatctctccaggcaagctcttccaccccttct
gcttgcatccagacaccatcaaacatgcaggctcagacacatgatatcaagctttttccccgtatccccccaggtgtctgcaggctca
aagagcagcgagaagcgttcagaggaaagcgatcccgtgccaccttccccgtgcccgggctgtccccgcacgctgccggctcggg
gatgcggggggagcgccggaccggagcggagccccgggcggctcgctgctgccccctagcgggggagggacgtaattacatcc
ctgggggctttgggggggggctgtccccgtgagctccccagatctgctttttgcctgtactgggtctctctggttagaccagatctgag
cctgggagctctctggctaactagggaacccactgcttaagcctcaataaagcttcagctgctcgagctagcagatctttttccctctg
ccaaaaattatggggacatcatgaagccccttgagcatctgacttctggctaataaaggaaatttattttcattgcaatagtgtgttgg
aattttttgtgtctctcactcggaaggacatatgggagggcaaatcatttaaaacatcagaatgagtatttggtttagagtttggcaac
atatgcccatatgctggctgccatgaacaaaggttggctataaagaggtcatcagtatatgaaacagccccctgctgtccattccttat
tccatagaaaagccttgacttgaggttagattttttttatattttgttttgtgttatttttttctttaacatccctaaaattttccttacatg
ttttactagccagatttttcctcctctcctgactactcccagtcatagctgtccctcttctcttatggagatccctcgacctgcagcccaagc
ttggcgtaatcatggtcatagctgtttcctgtgtgaaattgttatccgctcacaattccacacaacatacgagccggaagcataaagtgta
aagcctggggtgcctaatgagtgagctaactcacattaattgcgttgcgctcactgcccgctttccagtcgggaaacctgtcgtgcca
gcggatccgcatctcaattagtcagcaaccatagtcccgcccctaactccgcccatcccgcccctaactccgcccagttccgcccattc
tccgccccatggctgactaattttttttatttatgcagaggccgaggccgcctcggcctctgagctattccagaagtagtgaggaggct
tttttggaggcctaggcttttgcaaaaagctgtcgactgcagaggcctgcatgcaagcttggcgtaatcatggtcatagctgtttcctg
tgtgaaattgttatccgctcacaattccacacaacatacgagccggaagcataaagtgtaaagcctggggtgcctaatgagtgagct
aactcacattaattgcgttgcgctcactgcccgctttccagtcgggaaacctgtcgtgccagctgcattaatgaatcggccaacgcgc
ggggagaggcggtttgcgtattgggcgctcttccgcttcctcgctcactgactcgctgcgctcggtcgttcggctgcggcgagcggta
tcagctcactcaaaggcggtaatacggttatccacagaatcaggggataacgcaggaaagaacatgtgagcaaaaggccagcaa
aaggccaggaaccgtaaaaaggccgcgttgctggcgtttttccataggctccgcccccctgacgagcatcacaaaaatcgacgctc
aagtcagaggtggcgaaacccgacaggactataaagataccaggcgtttccccctggaagctccctcgtgcgctctcctgttccgac
cctgccgcttaccggatacctgtccgcctttctcccttcgggaagcgtggcgctttctcatagctcacgctgtaggtatctcagttcggtg
taggtcgttcgctccaagctgggctgtgtgcacgaaccccccgttcagcccgaccgctgcgccttatccggtaactatcgtcttgagtc
caacccggtaagacacgacttatcgccactggcagcagccactggtaacaggattagcagagcgaggtatgtaggcggtgctaca
gagttcttgaagtggtggcctaactacggctacactagaagaacagtatttggtatctgcgctctgctgaagccagttaccttcggaa
aaagagttggtagctcttgatccggcaaacaaaccaccgctggtagcggtggtttttttgtttgcaagcagcagattacgcgcagaa
aaaaaggatctcaagaagatcctttgatcttttctacggggtctgacgctcagtggaacgaaaactcacgttaagggattttggtcat
gagattatcaaaaaggatcttcacctagatccttttaaattaaaaatgaagttttaaatcaatctaaagtatatatgagtaaacttggt
ctgacagttaccaatgcttaatcagtgaggcacctatctcagcgatctgtctatttcgttcatccatagttgcctgactccccgtcgtgta
gataactacgatacgggagggcttaccatctggccccagtgctgcaatgataccgcgagacccacgctcaccggctccagatttatc
agcaataaaccagccagccggaagggccgagcgcagaagtggtcctgcaactttatccgcctccatccagtctattaattgttgccg
ggaagctagagtaagtagttcgccagttaatagtttgcgcaacgttgttgccattgctacaggcatcgtggtgtcacgctcgtcgtttg
gtatggcttcattcagctccggttcccaacgatcaaggcgagttacatgatcccccatgttgtgcaaaaaagcggttagctccttcggt
cctccgatcgttgtcagaagtaagttggccgcagtgttatcactcatggttatggcagcactgcataattctcttactgtcatgccatcc
gtaagatgcttttctgtgactggtgagtactcaaccaagtcattctgagaatagtgtatgcggcgaccgagttgctcttgcccggcgtc
aatacgggataataccgcgccacatagcagaactttaaaagtgctcatcattggaaaacgttcttcggggcgaaaactctcaaggat
cttaccgctgttgagatccagttcgatgtaacccactcgtgcacccaactgatcttcagcatcttttactttcaccagcgtttctgggtga
gcaaaaacaggaaggcaaaatgccgcaaaaaagggaataagggcgacacggaaatgttgaatactcatactcttcctttttcaat
attattgaagcatttatcagggttattgtctcatgagcggatacatatttgaatgtatttagaaaaataaacaaataggggttccgcgc
acatttccccgaaaagtgccacctgacgtctaagaaaccattattatcatgacattaacctataaaaataggcgtatcacgaggccct
ttcgtctcgcgcgtttcggtgatgacggtgaaaacctctgacacatgcagctcccggagacggtcacagcttgtctgtaagcggatgc
cgggagcagacaagcccgtcagggcgcgtcagcgggtgttggcgggtgtcggggctggcttaactatgcggcatcagagcagatt
gtactgagagtgcaccatatgcggtgtgaaataccgcacagatgcgtaaggagaaaataccgcatcaggcgccattcgccattcag
gctgcgcaactgttgggaagggcgatcggtgcgggcctcttcgctattacgccagctggcgaaagggggatgtgctgcaaggcga
ttaagttgggtaacgccagggttttcccagtcacgacgttgtaaaacgacggccagtgaattc
pBRNGTR84 pTL20c SK734rev MND WAS 650 (SEQ ID NO: 56)
ggccgcctcggccaaacagcccttgagtttaccactccctatcagtgatagagaaaagtgaaagtcgagtttaccactccctatcagt
gatagagaaaagtgaaagtcgagtttaccactccctatcagtgatagagaaaagtgaaagtcgagtttaccactccctatcagtgat
agagaaaagtgaaagtcgagtttaccagtccctatcagtgatagagaaaagtgaaagtcgagtttaccactccctatcagtgatag
agaaaagtgaaagtcgagtttaccactccctatcagtgatagagaaaagtgaaagtcgagctcgccatgggaggcgtggcctggg
cgggactggggagtggcgagccctcagatcctgcatataagcagctgctttttgcctgtactgggtctctctggttagaccagatctg
agcctgggagctctctggctaactagggaacccactgcttaagcctcaataaagcttgccttgagtgcttcaagtagtgtgtgcccgt
ctgttgtgtgactctggtaactagagatccctcagacccttttagtcagtgtggaaaatctctagcagtggcgcccgaacagggactt
gaaagcgaaagggaaaccagaggagctctctcgacgcaggactcggcttgctgaagcgcgcacggcaagaggcgaggggcgg
cgactggtgagtacgccaaaaattttgactagcggaggctagaaggagagagatgggtgcgagagcgtcagtattaagcggggg
agaattagatcgcgatgggaaaaaattcggttaaggccagggggaaagaaaaaatataaattaaaacatatagtatgggcaagc
agggagctagaacgattcgcagttaatactggcctgttagaaacatcagaaggctgtagacaaatactgggacagctacaaccatc
ccttcagacaggatcagaagaacttagatcattatataatacagtagcaaccctctattgtgtgcatcaaaggatagagataaaaga
caccaaggaagctttagacaagatagaggaagagcaaaaaaaagtaagaaaaaagcacagcaagcagcaggatcttcagac
ctggaaattccctacaatccccaaagtcaaggagtagtagaatctatgaataaagaattaaagaaaattataggacaggtaagag
atcaggctgaacatcttaagacagcagtacaaatggcagtattcatccacaattttaaaagaaaaggggggattggggggtacagt
gcaggggaaagaatagtagacataatagcaacagacatacaaactaaagaattacaaaaacaaattacaaaaattcaaaattttc
gggtttattacagggacagcagaaatccactttggaaaggaccagcaaagctcctctggaaaggtgaaggggcagtagtaataca
agataatagtgacataaaagtagtgccaagaagaaaagcaaagatcattagggattatggaaaacagatggcaggtgatgattg
tgtggcaagtagacaggatgaggattagaacatggaaaagtttagtaaaacaccataaggaggagatatgagggacaattggag
aagtgaattatataaatataaagtagtaaaaattgaaccattaggagtagcacccaccaaggcaaagagaagagtggtgcagag
agaaaaaagagcagtgggaataggagctttgttccttgggttcttgggagcagcaggaagcactatgggcgcagcgtcaatgacg
ctgacggtacaggccagacaattattgtctggtatagtgcagcagcagaacaatttgctgagggctattgaggcgcaacagcatct
gttgcaactcacagtctggggcatcaagcagctccaggcaagaatcctggctgtggaaagatacctaaaggatcaacagctcctgg
ggatttggggttgctctggaaaactcatttgcaccactgctgtgccttggaatgctagttggagtaataaatctctggaacagatttgg
aatcacacgacctggatggagtgggacagagaaattaacaattacacaagcttaatacactccttaattgaagaatcgcaaaacca
gcaagaaaagaatgaacaagaattattggaattagataaatgggcaagtttgtggaattggtttaacataacaaattggctgtggt
atataaaattattcataatgatagtaggaggcttggtaggtttaagaatagtttttgctgtactttctatagtgaatagagttaggcag
ggatattcaccattatcgtttcagacccacctcccaaccccgaggggaccgagctcaagcttcgaagcgatcgcacgcgtcaaaaa
aggatatgcccttgactatgtcggacaaatagtcaagggcatatcctgaggtacccaggcggcgcacaagctatataaacctgaag
gaaatctcaactttacacttaggtcaagttacttatcgtactagagcttcagcaggaaatttaactaaaatctaatttaaccagcatag
caaatatcatttattcccaaaatgctaaagtttgagataaacggacttgatttccggctgttttgacactatccagaatgccttgcagat
gggtggggcatgctaaatactgcacgtcgatacgcgtggatccgaacagagagacagcagaatatgggccaaacaggatatctgt
ggtaagcagttcctgccccggctcagggccaagaacagttggaacagcagaatatgggccaaacaggatatctgtggtaagcagt
tcctgccccggctcagggccaagaacagatggtccccagatgcggtcccgccctcagcagtttctagagaaccatcagatgtttcca
gggtgccccaaggacctgaaatgaccctgtgccttatttgaactaaccaatcagttcgcttctcgcttctgttcgcgcgcttctgctcccc
gagctctatataagcagagctcgtttagtgaaccgtcagatcggcgcgccaattcaagcgagaagacaagggcagccgccaccat
gagtgggggcccaatgggaggaaggcccgggggccgaggagcaccagcggttcagcagaacataccctccaccctcctccagg
accacgagaaccagcgactctttgagatgcttggacgaaaatgcttgacgctggccactgcagttgttcagctgtacctggcgctgc
cccctggagctgagcactggaccaaggagcattgtggggctgtgtgcttcgtgaaggataacccccagaagtcctacttcatccgcc
tttacggccttcaggctggtcggctgctctgggaacaggagctgtactcacagcttgtctactccacccccacccccttcttccacacct
tcgctggagatgactgccaagcggggctgaactttgcagacgaggacgaggcccaggccttccgggcactcgtgcaggagaaga
tacaaaaaaggaatcagaggcaaagtggagacagacgccagctacccccaccaccaacaccagccaatgaagagagaagagg
agggctcccacccctgcccctgcatccaggtggagaccaaggaggccctccagtgggtccgctctccctggggctggcgacagtgg
acatccagaaccctgacatcacgagttcacgataccgtgggctcccagcacctggacctagcccagctgataagaaacgctcaggg
aagaagaagatcagcaaagctgatattggtgcacccagtggattcaagcatgtcagccacgtggggtgggacccccagaatggat
ttgacgtgaacaacctcgacccagatctgcggagtctgttctccagggcaggaatcagcgaggcccagctcaccgacgccgagacc
tctaaacttatctacgacttcattgaggaccagggtgggctggaggctgtgcggcaggagatgaggcgccaggagccacttccgcc
gcccccaccgccatctcgaggagggaaccagctcccccggccccctattgtggggggtaacaagggtcgttctggtccactgccccc
tgtacctttggggattgccccacccccaccaacaccccggggacccccacccccaggccgagggggtcctccaccaccaccccctcc
agctactggacgttctggaccactgccccctccaccccctggagctggtgggccacccatgccaccaccaccgccaccaccgccacc
gccgcccagctccgggaatggaccagcccctcccccactccctcctgctctggtgcctgccgggggcctggcccctggtgggggtcg
gggagcgcttttggatcaaatccggcagggaattcagctgaacaagacccctggggccccagagagctcagcgctgcagccacca
cctcagagctcagagggactggtgggggccctgatgcacgtgatgcagaagagaagcagagccatccactcctccgacgaaggg
gaggaccaggctggcgatgaagatgaagatgatgaatgggatgactgataactagtaatcaacctctggattacaaaatttgtgaa
agattgactggtattcttaactatgttgctccttttacgctatgtggatacgctgctttaatgcctttgtatcatgctattgcttcccgtatg
gctttcattttctcctccttgtataaatcctggttgctgtctctttatgaggagttgtggcccgttgtcaggcaacgtggcgtggtgtgcac
tgtgtttgctgacgcaacccccactggttggggcattgccaccacctgtcagctcctttccgggactttcgctttccccctccctattgcc
acggcggaactcatcgccgcctgccttgcccgctgctggacaggggctcggctgttgggcactgacaattccgtggtgttgtcgggg
aaatcatcgtcctttccttggctgctcgcctgtgttgccacctggattctgcgcgggacgtccttctgctacgtcccttcggccctcaatcc
agcggaccttccttcccgcggcctgctgccggctctgcggcctcttccgcgtcttcgccttcgccctcagacgagtcggatctccctttg
ggccgcctccccgcacgtacgaccggtgcggccgcatcgatgccgtagtacctttaagaccaatgacttacaaggcagctgtagatc
ttagccactttttaaaagaaaaggggggactggaagggctaattcactcccaaagaagacaagatagccccatcctcactgactcc
gtcctggagttggatgagagataatggccttacgttgtgccaggggagggtcgggctggatttagcaagatttaccttctccaaaga
gcggtgctgcagtggcacagctgcccacggaggtgggggggtcaccgtccctggaggtgatgaagaactgtggggatgtggcac
tgagggacatggccagtgggcacggtgggtgggttggggttggtcttggggatcttggagggcttttccagccttcatgatttgacg
attgtatgaacatctacatggcaattctccagctgcctgtcccagtcctactgacccagctgtatctctccaggcaagctcttccacccct
tctgcttgcatccagacaccatcaaacatgcaggctcagacacatgatatcaagctttttccccgtatccccccaggtgtctgcaggct
caaagagcagcgagaagcgttcagaggaaagcgatcccgtgccaccttccccgtgcccgggctgtccccgcacgctgccggctcg
gggatgcggggggagcgccggaccggagcggagccccgggcggctcgctgctgccccctagcgggggagggacgtaattacat
ccctgggggctttgggggggggctgtccccgtgagctccccagatctgctttttgcctgtactgggtctctctggttagaccagatctg
agcctgggagctctctggctaactagggaacccactgcttaagcctcaataaagcttcagctgctcgagctagcagatctttttccctc
tgccaaaaattatggggacatcatgaagccccttgagcatctgacttctggctaataaaggaaatttattttcattgcaatagtgtgtt
ggaattttttgtgtctctcactcggaaggacatatgggagggcaaatcatttaaaacatcagaatgagtatttggtttagagtttggca
acatatgcccatatgctggctgccatgaacaaaggttggctataaagaggtcatcagtatatgaaacagccccctgctgtccattcct
tattccatagaaaagccttgacttgaggttagattttttttatattttgttttgtgttatttttttctttaacatccctaaaattttccttac
atgttttactagccagatttttcctcctctcctgactactcccagtcatagctgtccctcttctcttatggagatccctcgacctgcagccca
agcttggcgtaatcatggtcatagctgtttcctgtgtgaaattgttatccgctcacaattccacacaacatacgagccggaagcataaagtgt
aaagcctggggtgcctaatgagtgagctaactcacattaattgcgttgcgctcactgcccgctttccagtcgggaaacctgtcgtgcc
agcggatccgcatctcaattagtcagcaaccatagtcccgcccctaactccgcccatcccgcccctaactccgcccagttccgcccatt
ctccgccccatggctgactaattttttttatttatgcagaggccgaggccgcctcggcctctgagctattccagaagtagtgaggaggc
ttttttggaggcctaggcttttgcaaaaagctgtcgactgcagaggcctgcatgcaagcttggcgtaatcatggtcatagctgtttcct
gtgtgaaattgttatccgctcacaattccacacaacatacgagccggaagcataaagtgtaaagcctggggtgcctaatgagtgag
ctaactcacattaattgcgttgcgctcactgcccgctttccagtcgggaaacctgtcgtgccagctgcattaatgaatcggccaacgcg
cggggagaggcggtttgcgtattgggcgctcttccgcttcctcgctcactgactcgctgcgctcggtcgttcggctgcggcgagcggt
atcagctcactcaaaggcggtaatacggttatccacagaatcaggggataacgcaggaaagaacatgtgagcaaaaggccagca
aaaggccaggaaccgtaaaaaggccgcgttgctggcgtttttccataggctccgcccccctgacgagcatcacaaaaatcgacgct
caagtcagaggtggcgaaacccgacaggactataaagataccaggcgtttccccctggaagctccctcgtgcgctctcctgttccga
ccctgccgcttaccggatacctgtccgcctttctcccttcgggaagcgtggcgctttctcatagctcacgctgtaggtatctcagttcggt
gtaggtcgttcgctccaagctgggctgtgtgcacgaaccccccgttcagcccgaccgctgcgccttatccggtaactatcgtcttgagt
ccaacccggtaagacacgacttatcgccactggcagcagccactggtaacaggattagcagagcgaggtatgtaggcggtgctac
agagttcttgaagtggtggcctaactacggctacactagaagaacagtatttggtatctgcgctctgctgaagccagttaccttcgga
aaaagagttggtagctcttgatccggcaaacaaaccaccgctggtagcggtggtttttttgtttgcaagcagcagattacgcgcaga
aaaaaaggatctcaagaagatcctttgatcttttctacggggtctgacgctcagtggaacgaaaactcacgttaagggattttggtca
tgagattatcaaaaaggatcttcacctagatccttttaaattaaaaatgaagttttaaatcaatctaaagtatatatgagtaaacttggt
ctgacagttaccaatgcttaatcagtgaggcacctatctcagcgatctgtctatttcgttcatccatagttgcctgactccccgtcgtgta
gataactacgatacgggagggcttaccatctggccccagtgctgcaatgataccgcgagacccacgctcaccggctccagatttatc
agcaataaaccagccagccggaagggccgagcgcagaagtggtcctgcaactttatccgcctccatccagtctattaattgttgccg
ggaagctagagtaagtagttcgccagttaatagtttgcgcaacgttgttgccattgctacaggcatcgtggtgtcacgctcgtcgtttg
gtatggcttcattcagctccggttcccaacgatcaaggcgagttacatgatcccccatgttgtgcaaaaaagcggttagctccttcggt
cctccgatcgttgtcagaagtaagttggccgcagtgttatcactcatggttatggcagcactgcataattctcttactgtcatgccatcc
gtaagatgcttttctgtgactggtgagtactcaaccaagtcattctgagaatagtgtatgcggcgaccgagttgctcttgcccggcgtc
aatacgggataataccgcgccacatagcagaactttaaaagtgctcatcattggaaaacgttcttcggggcgaaaactctcaaggat
cttaccgctgttgagatccagttcgatgtaacccactcgtgcacccaactgatcttcagcatcttttactttcaccagcgtttctgggtga
gcaaaaacaggaaggcaaaatgccgcaaaaaagggaataagggcgacacggaaatgttgaatactcatactcttcctttttcaat
attattgaagcatttatcagggttattgtctcatgagcggatacatatttgaatgtatttagaaaaataaacaaataggggttccgcgc
acatttccccgaaaagtgccacctgacgtctaagaaaccattattatcatgacattaacctataaaaataggcgtatcacgaggccct
ttcgtctcgcgcgtttcggtgatgacggtgaaaacctctgacacatgcagctcccggagacggtcacagcttgtctgtaagcggatgc
cgggagcagacaagcccgtcagggcgcgtcagcgggtgttggcgggtgtcggggctggcttaactatgcggcatcagagcagatt
gtactgagagtgcaccatatgcggtgtgaaataccgcacagatgcgtaaggagaaaataccgcatcaggcgccattcgccattcag
gctgcgcaactgttgggaagggcgatcggtgcgggcctcttcgctattacgccagctggcgaaagggggatgtgctgcaaggcga
ttaagttgggtaacgccagggttttcccagtcacgacgttgtaaaacgacggccagtgaattc
pBRNGTR88 pTL20c SK734rev MND WAS 650 SAmut (SEQ ID NO: 57)
ggccgcctcggccaaacagcccttgagtttaccactccctatcagtgatagagaaaagtgaaagtcgagtttaccactccctatcagt
gatagagaaaagtgaaagtcgagtttaccactccctatcagtgatagagaaaagtgaaagtcgagtttaccactccctatcagtgat
agagaaaagtgaaagtcgagtttaccagtccctatcagtgatagagaaaagtgaaagtcgagtttaccactccctatcagtgatag
agaaaagtgaaagtcgagtttaccactccctatcagtgatagagaaaagtgaaagtcgagctcgccatgggaggcgtggcctggg
cgggactggggagtggcgagccctcagatcctgcatataagcagctgctttttgcctgtactgggtctctctggttagaccagatctg
agcctgggagctctctggctaactagggaacccactgcttaagcctcaataaagcttgccttgagtgcttcaagtagtgtgtgcccgt
ctgttgtgtgactctggtaactagagatccctcagacccttttagtcagtgtggaaaatctctagcagtggcgcccgaacagggactt
gaaagcgaaagggaaaccagaggagctctctcgacgcaggactcggcttgctgaagcgcgcacggcaagaggcgaggggcgg
cgactggtgagtacgccaaaaattttgactagcggaggctagaaggagagagatgggtgcgagagcgtcagtattaagcggggg
agaattagatcgcgatgggaaaaaattcggttaaggccagggggaaagaaaaaatataaattaaaacatatagtatgggcaagc
agggagctagaacgattcgcagttaatactggcctgttagaaacatcagaaggctgtagacaaatactgggacagctacaaccatc
ccttcagacaggatcagaagaacttagatcattatataatacagtagcaaccctctattgtgtgcatcaaaggatagagataaaaga
caccaaggaagctttagacaagatagaggaagagcaaaaaaaagtaagaaaaaagcacagcaagcagcaggatcttcagac
ctggaaattccctacaatccccaaagtcaaggagtagtagaatctatgaataaagaattaaagaaaattataggacaggtaagag
atcaggctgaacatcttaagacagcagtacaaatggcagtattcatccacaattttaaaagaaaaggggggattggggggtacagt
gcaggggaaagaatagtagacataatagcaacagacatacaaactaaagaattacaaaaacaaattacaaaaattcaaaattttc
gggtttattacagggacagcagaaatccactttggaaaggaccagcaaagctcctctggaaaggtgaaggggcagtagtaataca
agataatagtgacataaaagtagtgccaagaagaaaagcaaagatcattagggattatggaaaacagatggcaggtgatgattg
tgtggcaagtagacaggatgaggattagaacatggaaaagtttagtaaaacaccataaggaggagatatgagggacaattggag
aagtgaattatataaatataaagtagtaaaaattgaaccattaggagtagcacccaccaaggcaaagagaagagtggtgcagag
agaaaaaagagcagtgggaataggagctttgttccttgggttcttgggagcagcaggaagcactatgggcgcagcgtcaatgacg
ctgacggtacaggccagacaattattgtctggtatagtgcagcagcagaacaatttgctgagggctattgaggcgcaacagcatct
gttgcaactcacagtctggggcatcaagcagctccaggcaagaatcctggctgtggaaagatacctaaaggatcaacagctcctgg
ggatttggggttgctctggaaaactcatttgcaccactgctgtgccttggaatgctagttggagtaataaatctctggaacagatttgg
aatcacacgacctggatggagtgggacagagaaattaacaattacacaagcttaatacactccttaattgaagaatcgcaaaacca
gcaagaaaagaatgaacaagaattattggaattagataaatgggcaagtttgtggaattggtttaacataacaaattggctgtggt
atataaaattattcataatgatagtaggaggcttggtaggtttaagaatagtttttgctgtactttctatagtgaatagagttaggcag
ggatattcaccattatcgtttcagacccacctcccaaccccgaggggaccgagctcaagcttcgaagcgatcgcacgcgtcaaaaa
aggatatgcccttgactatgtcggacaaatagtcaagggcatatcctgaggtacccaggcggcgcacaagctatataaacctgaag
gaaatctcaactttacacttaggtcaagttacttatcgtactagagcttcagcaggaaatttaactaaaatctaatttaaccagcatag
caaatatcatttattcccaaaatgctaaagtttgagataaacggacttgatttccggctgttttgacactatccagaatgccttgcagat
gggtggggcatgctaaatactgcacgtcgatacgcgtggatccgaacagagagacagcagaatatgggccaaacaggatatctgt
ggtaagcagttcctgccccggctcagggccaagaacagttggaacagcagaatatgggccaaacaggatatctgtggtaagcagt
tcctgccccggctcagggccaagaacagatggtccccagatgcggtcccgccctcagcagtttctagagaaccatcagatgtttcca
gggtgccccaaggacctgaaatgaccctgtgccttatttgaactaaccaatcagttcgcttctcgcttctgttcgcgcgcttctgctcccc
gagctctatataagcagagctcgtttagtgaaccgtcagatcggcgcgccaattcaagcgagaagacaagggcagccgccaccat
gagtgggggcccaatgggaggaaggcccgggggccgaggagcaccagcggttcagcagaacataccctccaccctcctccagg
accacgagaaccagcgactctttgagatgcttggacgaaaatgcttgacgctggccactgcagttgttcagctgtacctggcgctgc
cccctggagctgagcactggaccaaggagcattgtggggctgtgtgcttcgtgaaggataacccccagaagtcctacttcatccgcc
tttacggccttcaggctggtcggctgctctgggaacaggagctgtactcacagcttgtctactccacccccacccccttcttccacacct
tcgctggagatgactgccaagcggggctgaactttgcagacgaggacgaggcccaggccttccgggcactcgtgcaggagaaga
tacaaaaaaggaatcagaggcaaagtggagacagacgccagctacccccaccaccaacaccagccaatgaagagagaagagg
agggctcccacccctgcccctgcatccaggtggagaccaaggaggccctccagtgggtccgctctccctggggctggcgacagtgg
acatccagaaccctgacatcacgagttcacgataccgtgggctcccagcacctggacctagcccagctgataagaaacgctcaggg
aagaagaagatcagcaaagctgatattggtgcacccagtggattcaagcatgtcagccacgtggggtgggacccccagaatggat
ttgacgtgaacaacctcgacccagatctgcggagtctgttctccagggcaggaatcagcgaggcccagctcaccgacgccgagacc
tctaaacttatctacgacttcattgaggaccagggtgggctggaggctgtgcggcaggagatgaggcgccaggagccacttccgcc
gcccccaccgccatctcgaggagggaaccagctcccccggccccctattgtggggggtaacaagggtcgttctggtccactgccccc
tgtacctttggggattgccccacccccaccaacaccccggggacccccacccccaggccgagggggtcctccaccaccaccccctcc
agctactggacgttctggaccactgccccctccaccccctggagctggtgggccacccatgccaccaccaccgccaccaccgccacc
gccgcccagctccgggaatggaccagcccctcccccactccctcctgctctggtgcctgccgggggcctggcccctggtgggggtcg
gggagcgcttttggatcaaatccggcagggaattcagctgaacaagacccctggggccccagagagctcagcgctgcagccacca
cctcagagctcagagggactggtgggggccctgatgcacgtgatgcagaagagaagcagagccatccactcctccgacgaaggg
gaggaccaggctggcgatgaagatgaagatgatgaatgggatgactgataactagtaatcaacctctggattacaaaatttgtgaa
agattgactggtattcttaactatgttgctccttttacgctatgtggatacgctgctttaatgcctttgtatcatgctattgcttcccgtatg
gctttcattttctcctccttgtataaatcctggttgctgtctctttatgaggagttgtggcccgttgtcaggcaacgtggcgtggtgtgcac
tgtgtttgctgacgcaacccccactggttggggcattgccaccacctgtcagctcctttccgggactttcgctttccccctccctattgcc
acggcggaactcatcgccgcctgccttgcccgctgctggacaggggctcggctgttgggcactgacaattccgtggtgttgtcgggg
aaatcatcgtcctttccttggctgctcgcctgtgttgccacctggattctgcgcgggacgtccttctgctacgtcccttcggccctcaatcc
agcggaccttccttcccgcggcctgctgccggctctgcggcctcttccgcgtcttcgccttcgccctcagacgagtcggatctccctttg
ggccgcctccccgcacgtacgaccggtgcggccgcatcgatgccgtagtacctttaagaccaatgacttacaaggcagctgtagatc
ttagccactttttaaaagaaaaggggggactggaagggctaattcactcccaaagaagacaagatagccccatcctcactgactcc
gtcctggagttggatgagagataatggccttacgttgtgccaggggagggtcgggctggatttagcaagatttaccttctccaaaga
gcggtgctgcagtggcacagctgcccacggaggtgggggggtcaccgtccctggaggtgatgaagaactgtggggatgtggcac
tgagggacatggccagtgggcacggtgggtgggttggggttggtcttggggatcttggagggcttttccagccttcatgatttgacg
attgtatgaacatctacatggcaattctccagctgcctgtcccagtcctactgacccagctgtatctctccaggcaagctcttccacccct
tctgcttgcatcctgacaccatcaaacatgcaggctcagacacatgatatcaagctttttccccgtatcccccctggtgtctgcaggctc
aaagagcagcgagaagcgttcagaggaaagcgatcccgtgccaccttccccgtgcccgggctgtccccgcacgctgccggctcgg
ggatgcggggggagcgccggaccggagcggagccccgggcggctcgctgctgccccctagcgggggagggacgtaattacatc
cctgggggctttgggggggggctgtccccgtgagctccccagatctgctttttgcctgtactgggtctctctggttagaccagatctga
gcctgggagctctctggctaactagggaacccactgcttaagcctcaataaagcttcagctgctcgagctagcagatctttttccctct
gccaaaaattatggggacatcatgaagccccttgagcatctgacttctggctaataaaggaaatttattttcattgcaatagtgtgttg
gaattttttgtgtctctcactcggaaggacatatgggagggcaaatcatttaaaacatcagaatgagtatttggtttagagtttggcaa
catatgcccatatgctggctgccatgaacaaaggttggctataaagaggtcatcagtatatgaaacagccccctgctgtccattcctt
attccatagaaaagccttgacttgaggttagattttttttatattttgttttgtgttatttttttctttaacatccctaaaattttccttaca
tgttttactagccagatttttcctcctctcctgactactcccagtcatagctgtccctcttctcttatggagatccctcgacctgcagcccaa
gcttggcgtaatcatggtcatagctgtttcctgtgtgaaattgttatccgctcacaattccacacaacatacgagccggaagcataaagtgt
aaagcctggggtgcctaatgagtgagctaactcacattaattgcgttgcgctcactgcccgctttccagtcgggaaacctgtcgtgcc
agcggatccgcatctcaattagtcagcaaccatagtcccgcccctaactccgcccatcccgcccctaactccgcccagttccgcccatt
ctccgccccatggctgactaattttttttatttatgcagaggccgaggccgcctcggcctctgagctattccagaagtagtgaggaggc
ttttttggaggcctaggcttttgcaaaaagctgtcgactgcagaggcctgcatgcaagcttggcgtaatcatggtcatagctgtttcct
gtgtgaaattgttatccgctcacaattccacacaacatacgagccggaagcataaagtgtaaagcctggggtgcctaatgagtgag
ctaactcacattaattgcgttgcgctcactgcccgctttccagtcgggaaacctgtcgtgccagctgcattaatgaatcggccaacgcg
cggggagaggcggtttgcgtattgggcgctcttccgcttcctcgctcactgactcgctgcgctcggtcgttcggctgcggcgagcggt
atcagctcactcaaaggcggtaatacggttatccacagaatcaggggataacgcaggaaagaacatgtgagcaaaaggccagca
aaaggccaggaaccgtaaaaaggccgcgttgctggcgtttttccataggctccgcccccctgacgagcatcacaaaaatcgacgct
caagtcagaggtggcgaaacccgacaggactataaagataccaggcgtttccccctggaagctccctcgtgcgctctcctgttccga
ccctgccgcttaccggatacctgtccgcctttctcccttcgggaagcgtggcgctttctcatagctcacgctgtaggtatctcagttcggt
gtaggtcgttcgctccaagctgggctgtgtgcacgaaccccccgttcagcccgaccgctgcgccttatccggtaactatcgtcttgagt
ccaacccggtaagacacgacttatcgccactggcagcagccactggtaacaggattagcagagcgaggtatgtaggcggtgctac
agagttcttgaagtggtggcctaactacggctacactagaagaacagtatttggtatctgcgctctgctgaagccagttaccttcgga
aaaagagttggtagctcttgatccggcaaacaaaccaccgctggtagcggtggtttttttgtttgcaagcagcagattacgcgcaga
aaaaaaggatctcaagaagatcctttgatcttttctacggggtctgacgctcagtggaacgaaaactcacgttaagggattttggtca
tgagattatcaaaaaggatcttcacctagatccttttaaattaaaaatgaagttttaaatcaatctaaagtatatatgagtaaacttggt
ctgacagttaccaatgcttaatcagtgaggcacctatctcagcgatctgtctatttcgttcatccatagttgcctgactccccgtcgtgta
gataactacgatacgggagggcttaccatctggccccagtgctgcaatgataccgcgagacccacgctcaccggctccagatttatc
agcaataaaccagccagccggaagggccgagcgcagaagtggtcctgcaactttatccgcctccatccagtctattaattgttgccg
ggaagctagagtaagtagttcgccagttaatagtttgcgcaacgttgttgccattgctacaggcatcgtggtgtcacgctcgtcgtttg
gtatggcttcattcagctccggttcccaacgatcaaggcgagttacatgatcccccatgttgtgcaaaaaagcggttagctccttcggt
cctccgatcgttgtcagaagtaagttggccgcagtgttatcactcatggttatggcagcactgcataattctcttactgtcatgccatcc
gtaagatgcttttctgtgactggtgagtactcaaccaagtcattctgagaatagtgtatgcggcgaccgagttgctcttgcccggcgtc
aatacgggataataccgcgccacatagcagaactttaaaagtgctcatcattggaaaacgttcttcggggcgaaaactctcaaggat
cttaccgctgttgagatccagttcgatgtaacccactcgtgcacccaactgatcttcagcatcttttactttcaccagcgtttctgggtga
gcaaaaacaggaaggcaaaatgccgcaaaaaagggaataagggcgacacggaaatgttgaatactcatactcttcctttttcaat
attattgaagcatttatcagggttattgtctcatgagcggatacatatttgaatgtatttagaaaaataaacaaataggggttccgcgc
acatttccccgaaaagtgccacctgacgtctaagaaaccattattatcatgacattaacctataaaaataggcgtatcacgaggccct
ttcgtctcgcgcgtttcggtgatgacggtgaaaacctctgacacatgcagctcccggagacggtcacagcttgtctgtaagcggatgc
cgggagcagacaagcccgtcagggcgcgtcagcgggtgttggcgggtgtcggggctggcttaactatgcggcatcagagcagatt
gtactgagagtgcaccatatgcggtgtgaaataccgcacagatgcgtaaggagaaaataccgcatcaggcgccattcgccattcag
gctgcgcaactgttgggaagggcgatcggtgcgggcctcttcgctattacgccagctggcgaaagggggatgtgctgcaaggcga
ttaagttgggtaacgccagggttttcccagtcacgacgttgtaaaacgacggccagtgaattc
pBRNGTR92 pTL20c SK734rev MND WAS 650fwd (SEQ ID NO: 58)
ggccgcctcggccaaacagcccttgagtttaccactccctatcagtgatagagaaaagtgaaagtcgagtttaccactccctatcagt
gatagagaaaagtgaaagtcgagtttaccactccctatcagtgatagagaaaagtgaaagtcgagtttaccactccctatcagtgat
agagaaaagtgaaagtcgagtttaccagtccctatcagtgatagagaaaagtgaaagtcgagtttaccactccctatcagtgatag
agaaaagtgaaagtcgagtttaccactccctatcagtgatagagaaaagtgaaagtcgagctcgccatgggaggcgtggcctggg
cgggactggggagtggcgagccctcagatcctgcatataagcagctgctttttgcctgtactgggtctctctggttagaccagatctg
agcctgggagctctctggctaactagggaacccactgcttaagcctcaataaagcttgccttgagtgcttcaagtagtgtgtgcccgt
ctgttgtgtgactctggtaactagagatccctcagacccttttagtcagtgtggaaaatctctagcagtggcgcccgaacagggactt
gaaagcgaaagggaaaccagaggagctctctcgacgcaggactcggcttgctgaagcgcgcacggcaagaggcgaggggcgg
cgactggtgagtacgccaaaaattttgactagcggaggctagaaggagagagatgggtgcgagagcgtcagtattaagcggggg
agaattagatcgcgatgggaaaaaattcggttaaggccagggggaaagaaaaaatataaattaaaacatatagtatgggcaagc
agggagctagaacgattcgcagttaatactggcctgttagaaacatcagaaggctgtagacaaatactgggacagctacaaccatc
ccttcagacaggatcagaagaacttagatcattatataatacagtagcaaccctctattgtgtgcatcaaaggatagagataaaaga
caccaaggaagctttagacaagatagaggaagagcaaaacaaaagtaagaaaaaagcacagcaagcagcaggatcttcagac
ctggaaattccctacaatccccaaagtcaaggagtagtagaatctatgaataaagaattaaagaaaattataggacaggtaagag
atcaggctgaacatcttaagacagcagtacaaatggcagtattcatccacaattttaaaagaaaaggggggattggggggtacagt
gcaggggaaagaatagtagacataatagcaacagacatacaaactaaagaattacaaaaacaaattacaaaaattcaaaattttc
gggtttattacagggacagcagaaatccactttggaaaggaccagcaaagctcctctggaaaggtgaaggggcagtagtaataca
agataatagtgacataaaagtagtgccaagaagaaaagcaaagatcattagggattatggaaaacagatggcaggtgatgattg
tgtggcaagtagacaggatgaggattagaacatggaaaagtttagtaaaacaccataaggaggagatatgagggacaattggag
aagtgaattatataaatataaagtagtaaaaattgaaccattaggagtagcacccaccaaggcaaagagaagagtggtgcagag
agaaaaaagagcagtgggaataggagctttgttccttgggttcttgggagcagcaggaagcactatgggcgcagcgtcaatgacg
ctgacggtacaggccagacaattattgtctggtatagtgcagcagcagaacaatttgctgagggctattgaggcgcaacagcatct
gttgcaactcacagtctggggcatcaagcagctccaggcaagaatcctggctgtggaaagatacctaaaggatcaacagctcctgg
ggatttggggttgctctggaaaactcatttgcaccactgctgtgccttggaatgctagttggagtaataaatctctggaacagatttgg
aatcacacgacctggatggagtgggacagagaaattaacaattacacaagcttaatacactccttaattgaagaatcgcaaaacca
gcaagaaaagaatgaacaagaattattggaattagataaatgggcaagtttgtggaattggtttaacataacaaattggctgtggt
atataaaattattcataatgatagtaggaggcttggtaggtttaagaatagtttttgctgtactttctatagtgaatagagttaggcag
ggatattcaccattatcgtttcagacccacctcccaaccccgaggggaccgagctcaagcttcgaagcgatcgcacgcgtcaaaaa
aggatatgcccttgactatgtcggacaaatagtcaagggcatatcctgaggtacccaggcggcgcacaagctatataaacctgaag
gaaatctcaactttacacttaggtcaagttacttatcgtactagagcttcagcaggaaatttaactaaaatctaatttaaccagcatag
caaatatcatttattcccaaaatgctaaagtttgagataaacggacttgatttccggctgttttgacactatccagaatgccttgcagat
gggtggggcatgctaaatactgcacgtcgatacgcgtggatccgaacagagagacagcagaatatgggccaaacaggatatctgt
ggtaagcagttcctgccccggctcagggccaagaacagttggaacagcagaatatgggccaaacaggatatctgtggtaagcagt
tcctgccccggctcagggccaagaacagatggtccccagatgcggtcccgccctcagcagtttctagagaaccatcagatgtttcca
gggtgccccaaggacctgaaatgaccctgtgccttatttgaactaaccaatcagttcgcttctcgcttctgttcgcgcgcttctgctcccc
gagctctatataagcagagctcgtttagtgaaccgtcagatcggcgcgccaattcaagcgagaagacaagggcagccgccaccat
gagtgggggcccaatgggaggaaggcccgggggccgaggagcaccagcggttcagcagaacataccctccaccctcctccagg
accacgagaaccagcgactctttgagatgcttggacgaaaatgcttgacgctggccactgcagttgttcagctgtacctggcgctgc
cccctggagctgagcactggaccaaggagcattgtggggctgtgtgcttcgtgaaggataacccccagaagtcctacttcatccgcc
tttacggccttcaggctggtcggctgctctgggaacaggagctgtactcacagcttgtctactccacccccacccccttcttccacacct
tcgctggagatgactgccaagcggggctgaactttgcagacgaggacgaggcccaggccttccgggcactcgtgcaggagaaga
tacaaaaaaggaatcagaggcaaagtggagacagacgccagctacccccaccaccaacaccagccaatgaagagagaagagg
agggctcccacccctgcccctgcatccaggtggagaccaaggaggccctccagtgggtccgctctccctggggctggcgacagtgg
acatccagaaccctgacatcacgagttcacgataccgtgggctcccagcacctggacctagcccagctgataagaaacgctcaggg
aagaagaagatcagcaaagctgatattggtgcacccagtggattcaagcatgtcagccacgtggggtgggacccccagaatggat
ttgacgtgaacaacctcgacccagatctgcggagtctgttctccagggcaggaatcagcgaggcccagctcaccgacgccgagacc
tctaaacttatctacgacttcattgaggaccagggtgggctggaggctgtgcggcaggagatgaggcgccaggagccacttccgcc
gcccccaccgccatctcgaggagggaaccagctcccccggccccctattgtggggggtaacaagggtcgttctggtccactgccccc
tgtacctttggggattgccccacccccaccaacaccccggggacccccacccccaggccgagggggtcctccaccaccaccccctcc
agctactggacgttctggaccactgccccctccaccccctggagctggtgggccacccatgccaccaccaccgccaccaccgccacc
gccgcccagctccgggaatggaccagcccctcccccactccctcctgctctggtgcctgccgggggcctggcccctggtgggggtcg
gggagcgcttttggatcaaatccggcagggaattcagctgaacaagacccctggggccccagagagctcagcgctgcagccacca
cctcagagctcagagggactggtgggggccctgatgcacgtgatgcagaagagaagcagagccatccactcctccgacgaaggg
gaggaccaggctggcgatgaagatgaagatgatgaatgggatgactgataactagtaatcaacctctggattacaaaatttgtgaa
agattgactggtattcttaactatgttgctccttttacgctatgtggatacgctgctttaatgcctttgtatcatgctattgcttcccgtatg
gctttcattttctcctccttgtataaatcctggttgctgtctctttatgaggagttgtggcccgttgtcaggcaacgtggcgtggtgtgcac
tgtgtttgctgacgcaacccccactggttggggcattgccaccacctgtcagctcctttccgggactttcgctttccccctccctattgcc
acggcggaactcatcgccgcctgccttgcccgctgctggacaggggctcggctgttgggcactgacaattccgtggtgttgtcgggg
aaatcatcgtcctttccttggctgctcgcctgtgttgccacctggattctgcgcgggacgtccttctgctacgtcccttcggccctcaatcc
agcggaccttccttcccgcggcctgctgccggctctgcggcctcttccgcgtcttcgccttcgccctcagacgagtcggatctccctttg
ggccgcctccccgcacgtacgaccggtgcggccgcatcgatgccgtagtacctttaagaccaatgacttacaaggcagctgtagatc
ttagccactttttaaaagaaaaggggggactggaagggctaattcactcccaaagaagacaagatggggagctcacggggacag
cccccccccaaagcccccagggatgtaattacgtccctcccccgctagggggcagcagcgagccgcccggggctccgctccggtcc
ggcgctccccccgcatccccgagccggcagcgtgcggggacagcccgggcacggggaaggtggcacgggatcgctttcctctgaa
cgcttctcgctgctctttgagcctgcagacacctggggggatacggggaaaaagcttgatatcatgtgtctgagcctgcatgtttgat
ggtgtctggatgcaagcagaaggggtggaagagcttgcctggagagatacagctgggtcagtaggactgggacaggcagctgg
agaattgccatgtagatgttcatacaatcgtcaaatcatgaaggctggaaaagccctccaagatccccaagaccaaccccaaccca
cccaccgtgcccactggccatgtccctcagtgccacatccccacagttcttcatcacctccagggacggtgacccccccacctccgtgg
gcagctgtgccactgcagcaccgctctttggagaaggtaaatcttgctaaatccagcccgaccctcccctggcacaacgtaaggcca
ttatctctcatccaactccaggacggagtcagtgaggatggggctagatctgctttttgcctgtactgggtctctctggttagaccagat
ctgagcctgggagctctctggctaactagggaacccactgcttaagcctcaataaagcttcagctgctcgagctagcagatctttttcc
ctctgccaaaaattatggggacatcatgaagccccttgagcatctgacttctggctaataaaggaaatttattttcattgcaatagtgt
gttggaattttttgtgtctctcactcggaaggacatatgggagggcaaatcatttaaaacatcagaatgagtatttggtttagagtttg
gcaacatatgcccatatgctggctgccatgaacaaaggttggctataaagaggtcatcagtatatgaaacagccccctgctgtccatt
ccttattccatagaaaagccttgacttgaggttagattttttttatattttgttttgtgttatttttttctttaacatccctaaaattttcct
tacatgttttactagccagatttttcctcctctcctgactactcccagtcatagctgtccctcttctcttatggagatccctcgacctgcagcc
caagcttggcgtaatcatggtcatagctgtttcctgtgtgaaattgttatccgctcacaattccacacaacatacgagccggaagcataaa
gtgtaaagcctggggtgcctaatgagtgagctaactcacattaattgcgttgcgctcactgcccgctttccagtcgggaaacctgtcgt
gccagcggatccgcatctcaattagtcagcaaccatagtcccgcccctaactccgcccatcccgcccctaactccgcccagttccgcc
cattctccgccccatggctgactaattttttttatttatgcagaggccgaggccgcctcggcctctgagctattccagaagtagtgagg
aggcttttttggaggcctaggcttttgcaaaaagctgtcgactgcagaggcctgcatgcaagcttggcgtaatcatggtcatagctgtt
tcctgtgtgaaattgttatccgctcacaattccacacaacatacgagccggaagcataaagtgtaaagcctggggtgcctaatgagt
gagctaactcacattaattgcgttgcgctcactgcccgctttccagtcgggaaacctgtcgtgccagctgcattaatgaatcggccaac
gcgcggggagaggcggtttgcgtattgggcgctcttccgcttcctcgctcactgactcgctgcgctcggtcgttcggctgcggcgagc
ggtatcagctcactcaaaggcggtaatacggttatccacagaatcaggggataacgcaggaaagaacatgtgagcaaaaggcca
gcaaaaggccaggaaccgtaaaaaggccgcgttgctggcgtttttccataggctccgcccccctgacgagcatcacaaaaatcgac
gctcaagtcagaggtggcgaaacccgacaggactataaagataccaggcgtttccccctggaagctccctcgtgcgctctcctgttc
cgaccctgccgcttaccggatacctgtccgcctttctcccttcgggaagcgtggcgctttctcatagctcacgctgtaggtatctcagttc
ggtgtaggtcgttcgctccaagctgggctgtgtgcacgaaccccccgttcagcccgaccgctgcgccttatccggtaactatcgtcttg
agtccaacccggtaagacacgacttatcgccactggcagcagccactggtaacaggattagcagagcgaggtatgtaggcggtgc
tacagagttcttgaagtggtggcctaactacggctacactagaagaacagtatttggtatctgcgctctgctgaagccagttaccttcg
gaaaaagagttggtagctcttgatccggcaaacaaaccaccgctggtagcggtggtttttttgtttgcaagcagcagattacgcgca
gaaaaaaaggatctcaagaagatcctttgatcttttctacggggtctgacgctcagtggaacgaaaactcacgttaagggattttggt
catgagattatcaaaaaggatcttcacctagatccttttaaattaaaaatgaagttttaaatcaatctaaagtatatatgagtaaacttg
gtctgacagttaccaatgcttaatcagtgaggcacctatctcagcgatctgtctatttcgttcatccatagttgcctgactccccgtcgtg
tagataactacgatacgggagggcttaccatctggccccagtgctgcaatgataccgcgagacccacgctcaccggctccagattta
tcagcaataaaccagccagccggaagggccgagcgcagaagtggtcctgcaactttatccgcctccatccagtctattaattgttgcc
gggaagctagagtaagtagttcgccagttaatagtttgcgcaacgttgttgccattgctacaggcatcgtggtgtcacgctcgtcgttt
ggtatggcttcattcagctccggttcccaacgatcaaggcgagttacatgatcccccatgttgtgcaaaaaagcggttagctccttcg
gtcctccgatcgttgtcagaagtaagttggccgcagtgttatcactcatggttatggcagcactgcataattctcttactgtcatgccat
ccgtaagatgcttttctgtgactggtgagtactcaaccaagtcattctgagaatagtgtatgcggcgaccgagttgctcttgcccggcg
tcaatacgggataataccgcgccacatagcagaactttaaaagtgctcatcattggaaaacgttcttcggggcgaaaactctcaagg
atcttaccgctgttgagatccagttcgatgtaacccactcgtgcacccaactgatcttcagcatcttttactttcaccagcgtttctgggt
gagcaaaaacaggaaggcaaaatgccgcaaaaaagggaataagggcgacacggaaatgttgaatactcatactcttcctttttca
atattattgaagcatttatcagggttattgtctcatgagcggatacatatttgaatgtatttagaaaaataaacaaataggggttccgc
gcacatttccccgaaaagtgccacctgacgtctaagaaaccattattatcatgacattaacctataaaaataggcgtatcacgaggcc
ctttcgtctcgcgcgtttcggtgatgacggtgaaaacctctgacacatgcagctcccggagacggtcacagcttgtctgtaagcggat
gccgggagcagacaagcccgtcagggcgcgtcagcgggtgttggcgggtgtcggggctggcttaactatgcggcatcagagcag
attgtactgagagtgcaccatatgcggtgtgaaataccgcacagatgcgtaaggagaaaataccgcatcaggcgccattcgccatt
caggctgcgcaactgttgggaagggcgatcggtgcgggcctcttcgctattacgccagctggcgaaagggggatgtgctgcaagg
cgattaagttgggtaacgccagggttttcccagtcacgacgttgtaaaacgacggccagtgaattc
pBRNGTR120 pTL20c SK734rev MND WAS 650 3xSAmut (SEQ ID NO: 59)
ggccgcctcggccaaacagcccttgagtttaccactccctatcagtgatagagaaaagtgaaagtcgagtttaccactccctatcagt
gatagagaaaagtgaaagtcgagtttaccactccctatcagtgatagagaaaagtgaaagtcgagtttaccactccctatcagtgat
agagaaaagtgaaagtcgagtttaccagtccctatcagtgatagagaaaagtgaaagtcgagtttaccactccctatcagtgatag
agaaaagtgaaagtcgagtttaccactccctatcagtgatagagaaaagtgaaagtcgagctcgccatgggaggcgtggcctggg
cgggactggggagtggcgagccctcagatcctgcatataagcagctgctttttgcctgtactgggtctctctggttagaccagatctg
agcctgggagctctctggctaactagggaacccactgcttaagcctcaataaagcttgccttgagtgcttcaagtagtgtgtgcccgt
ctgttgtgtgactctggtaactagagatccctcagacccttttagtcagtgtggaaaatctctagcagtggcgcccgaacagggactt
gaaagcgaaagggaaaccagaggagctctctcgacgcaggactcggcttgctgaagcgcgcacggcaagaggcgaggggcgg
cgactggtgagtacgccaaaaattttgactagcggaggctagaaggagagagatgggtgcgagagcgtcagtattaagcggggg
agaattagatcgcgatgggaaaaaattcggttaaggccagggggaaagaaaaaatataaattaaaacatatagtatgggcaagc
agggagctagaacgattcgcagttaatactggcctgttagaaacatcagaaggctgtagacaaatactgggacagctacaaccatc
ccttcagacaggatcagaagaacttagatcattatataatacagtagcaaccctctattgtgtgcatcaaaggatagagataaaaga
caccaaggaagctttagacaagatagaggaagagcaaaacaaaagtaagaaaaaagcacagcaagcagcaggatcttcagac
ctggaaattccctacaatccccaaagtcaaggagtagtagaatctatgaataaagaattaaagaaaattataggacaggtaagag
atcaggctgaacatcttaagacagcagtacaaatggcagtattcatccacaattttaaaagaaaaggggggattggggggtacagt
gcaggggaaagaatagtagacataatagcaacagacatacaaactaaagaattacaaaaacaaattacaaaaattcaaaattttc
gggtttattacagggacagcagaaatccactttggaaaggaccagcaaagctcctctggaaaggtgaaggggcagtagtaataca
agataatagtgacataaaagtagtgccaagaagaaaagcaaagatcattagggattatggaaaacagatggcaggtgatgattg
tgtggcaagtagacaggatgaggattagaacatggaaaagtttagtaaaacaccataaggaggagatatgagggacaattggag
aagtgaattatataaatataaagtagtaaaaattgaaccattaggagtagcacccaccaaggcaaagagaagagtggtgcagag
agaaaaaagagcagtgggaataggagctttgttccttgggttcttgggagcagcaggaagcactatgggcgcagcgtcaatgacg
ctgacggtacaggccagacaattattgtctggtatagtgcagcagcagaacaatttgctgagggctattgaggcgcaacagcatct
gttgcaactcacagtctggggcatcaagcagctccaggcaagaatcctggctgtggaaagatacctaaaggatcaacagctcctgg
ggatttggggttgctctggaaaactcatttgcaccactgctgtgccttggaatgctagttggagtaataaatctctggaacagatttgg
aatcacacgacctggatggagtgggacagagaaattaacaattacacaagcttaatacactccttaattgaagaatcgcaaaacca
gcaagaaaagaatgaacaagaattattggaattagataaatgggcaagtttgtggaattggtttaacataacaaattggctgtggt
atataaaattattcataatgatagtaggaggcttggtaggtttaagaatagtttttgctgtactttctatagtgaatagagttaggcag
ggatattcaccattatcgtttcagacccacctcccaaccccgaggggaccgagctcaagcttcgaagcgatcgcacgcgtcaaaaa
aggatatgcccttgactatgtcggacaaatagtcaagggcatatcctgaggtacccaggcggcgcacaagctatataaacctgaag
gaaatctcaactttacacttaggtcaagttacttatcgtactagagcttcagcaggaaatttaactaaaatctaatttaaccagcatag
caaatatcatttattcccaaaatgctaaagtttgagataaacggacttgatttccggctgttttgacactatccagaatgccttgcagat
gggtggggcatgctaaatactgcacgtcgatacgcgtggatccgaacagagagacagcagaatatgggccaaacaggatatctgt
ggtaagcagttcctgccccggctcagggccaagaacagttggaacagcagaatatgggccaaacaggatatctgtggtaagcagt
tcctgccccggctcagggccaagaacagatggtccccagatgcggtcccgccctcagcagtttctagagaaccatcagatgtttcca
gggtgccccaaggacctgaaatgaccctgtgccttatttgaactaaccaatcagttcgcttctcgcttctgttcgcgcgcttctgctcccc
gagctctatataagcagagctcgtttagtgaaccgtcagatcggcgcgccaattcaagcgagaagacaagggcagccgccaccat
gagtgggggcccaatgggaggaaggcccgggggccgaggagcaccagcggttcagcagaacataccctccaccctcctccagg
accacgagaaccagcgactctttgagatgcttggacgaaaatgcttgacgctggccactgcagttgttcagctgtacctggcgctgc
cccctggagctgagcactggaccaaggagcattgtggggctgtgtgcttcgtgaaggataacccccagaagtcctacttcatccgcc
tttacggccttcaggctggtcggctgctctgggaacaggagctgtactcacagcttgtctactccacccccacccccttcttccacacct
tcgctggagatgactgccaagcggggctgaactttgcagacgaggacgaggcccaggccttccgggcactcgtgcaggagaaga
tacaaaaaaggaatcagaggcaaagtggagacagacgccagctacccccaccaccaacaccagccaatgaagagagaagagg
agggctcccacccctgcccctgcatccaggtggagaccaaggaggccctccagtgggtccgctctccctggggctggcgacagtgg
acatccagaaccctgacatcacgagttcacgataccgtgggctcccagcacctggacctagcccagctgataagaaacgctcaggg
aagaagaagatcagcaaagctgatattggtgcacccagtggattcaagcatgtcagccacgtggggtgggacccccagaatggat
ttgacgtgaacaacctcgacccagatctgcggagtctgttctccagggcaggaatcagcgaggcccagctcaccgacgccgagacc
tctaaacttatctacgacttcattgaggaccagggtgggctggaggctgtgcggcaggagatgaggcgccaggagccacttccgcc
gcccccaccgccatctcgaggagggaaccagctcccccggccccctattgtggggggtaacaagggtcgttctggtccactgccccc
tgtacctttggggattgccccacccccaccaacaccccggggacccccacccccaggccgagggggtcctccaccaccaccccctcc
agctactggacgttctggaccactgccccctccaccccctggagctggtgggccacccatgccaccaccaccgccaccaccgccacc
gccgcccagctccgggaatggaccagcccctcccccactccctcctgctctggtgcctgccgggggcctggcccctggtgggggtcg
gggagcgcttttggatcaaatccggcagggaattcagctgaacaagacccctggggccccagagagctcagcgctgcagccacca
cctcagagctcagagggactggtgggggccctgatgcacgtgatgcagaagagaagcagagccatccactcctccgacgaaggg
gaggaccaggctggcgatgaagatgaagatgatgaatgggatgactgataactagtaatcaacctctggattacaaaatttgtgaa
agattgactggtattcttaactatgttgctccttttacgctatgtggatacgctgctttaatgcctttgtatcatgctattgcttcccgtatg
gctttcattttctcctccttgtataaatcctggttgctgtctctttatgaggagttgtggcccgttgtcaggcaacgtggcgtggtgtgcac
tgtgtttgctgacgcaacccccactggttggggcattgccaccacctgtcagctcctttccgggactttcgctttccccctccctattgcc
acggcggaactcatcgccgcctgccttgcccgctgctggacaggggctcggctgttgggcactgacaattccgtggtgttgtcgggg
aaatcatcgtcctttccttggctgctcgcctgtgttgccacctggattctgcgcgggacgtccttctgctacgtcccttcggccctcaatcc
agcggaccttccttcccgcggcctgctgccggctctgcggcctcttccgcgtcttcgccttcgccctcagacgagtcggatctccctttg
ggccgcctccccgcacgtacgaccggtgcggccgcatcgatgccgtagtacctttaagaccaatgacttacaaggcagctgtagatc
ttagccactttttaaaagaaaaggggggactggaagggctaattcactcccaaagaagacaagatagccccatcctcactgactcc
gtcctggagttggatgagagataatggccttacgttgtgccaggggagggtcgggctggatttagcaagatttaccttctccaaaga
gcggtgctgcagtggcacagctgcccacggaggtgggggggtcaccgtccctggaggtgatgaagaactgtggggatgtggcac
tgagggacatggccagtgggcacggtgggtgggttggggttggtcttggggatcttggagggcttttccagccttcatgatttgacg
attgtatgaacatctacatggcaattctccagctgcctgtcccagtcctactgacccagctgtatctctccaggcaagctcttccacccct
tctgcttgcatcctgacaccatcaaacatgcaggctcagacacatgatatcaagctttttccccgtatcccccctggtgtctgctggctc
aaagagcagcgagaagcgttcagaggaaagcgatcccgtgccaccttccccgtgcccgggctgtccccgcacgctgccggctcgg
ggatgcggggggagcgccggaccggagcggagccccgggcggctcgctgctgccccctagcgggggagggacgtaattacatc
cctgggggctttgggggggggctgtccccgtgagctccccagatctgctttttgcctgtactgggtctctctggttagaccagatctga
gcctgggagctctctggctaactagggaacccactgcttaagcctcaataaagcttcagctgctcgagctagcagatctttttccctct
gccaaaaattatggggacatcatgaagccccttgagcatctgacttctggctaataaaggaaatttattttcattgcaatagtgtgttg
gaattttttgtgtctctcactcggaaggacatatgggagggcaaatcatttaaaacatcagaatgagtatttggtttagagtttggcaa
catatgcccatatgctggctgccatgaacaaaggttggctataaagaggtcatcagtatatgaaacagccccctgctgtccattcctt
attccatagaaaagccttgacttgaggttagattttttttatattttgttttgtgttatttttttctttaacatccctaaaattttccttaca
tgttttactagccagatttttcctcctctcctgactactcccagtcatagctgtccctcttctcttatggagatccctcgacctgcagcccaa
gcttggcgtaatcatggtcatagctgtttcctgtgtgaaattgttatccgctcacaattccacacaacatacgagccggaagcataaagtgt
aaagcctggggtgcctaatgagtgagctaactcacattaattgcgttgcgctcactgcccgctttccagtcgggaaacctgtcgtgcc
agcggatccgcatctcaattagtcagcaaccatagtcccgcccctaactccgcccatcccgcccctaactccgcccagttccgcccatt
ctccgccccatggctgactaattttttttatttatgcagaggccgaggccgcctcggcctctgagctattccagaagtagtgaggaggc
ttttttggaggcctaggcttttgcaaaaagctgtcgactgcagaggcctgcatgcaagcttggcgtaatcatggtcatagctgtttcct
gtgtgaaattgttatccgctcacaattccacacaacatacgagccggaagcataaagtgtaaagcctggggtgcctaatgagtgag
ctaactcacattaattgcgttgcgctcactgcccgctttccagtcgggaaacctgtcgtgccagctgcattaatgaatcggccaacgcg
cggggagaggcggtttgcgtattgggcgctcttccgcttcctcgctcactgactcgctgcgctcggtcgttcggctgcggcgagcggt
atcagctcactcaaaggcggtaatacggttatccacagaatcaggggataacgcaggaaagaacatgtgagcaaaaggccagca
aaaggccaggaaccgtaaaaaggccgcgttgctggcgtttttccataggctccgcccccctgacgagcatcacaaaaatcgacgct
caagtcagaggtggcgaaacccgacaggactataaagataccaggcgtttccccctggaagctccctcgtgcgctctcctgttccga
ccctgccgcttaccggatacctgtccgcctttctcccttcgggaagcgtggcgctttctcatagctcacgctgtaggtatctcagttcggt
gtaggtcgttcgctccaagctgggctgtgtgcacgaaccccccgttcagcccgaccgctgcgccttatccggtaactatcgtcttgagt
ccaacccggtaagacacgacttatcgccactggcagcagccactggtaacaggattagcagagcgaggtatgtaggcggtgctac
agagttcttgaagtggtggcctaactacggctacactagaagaacagtatttggtatctgcgctctgctgaagccagttaccttcgga
aaaagagttggtagctcttgatccggcaaacaaaccaccgctggtagcggtggtttttttgtttgcaagcagcagattacgcgcaga
aaaaaaggatctcaagaagatcctttgatcttttctacggggtctgacgctcagtggaacgaaaactcacgttaagggattttggtca
tgagattatcaaaaaggatcttcacctagatccttttaaattaaaaatgaagttttaaatcaatctaaagtatatatgagtaaacttggt
ctgacagttaccaatgcttaatcagtgaggcacctatctcagcgatctgtctatttcgttcatccatagttgcctgactccccgtcgtgta
gataactacgatacgggagggcttaccatctggccccagtgctgcaatgataccgcgagacccacgctcaccggctccagatttatc
agcaataaaccagccagccggaagggccgagcgcagaagtggtcctgcaactttatccgcctccatccagtctattaattgttgccg
ggaagctagagtaagtagttcgccagttaatagtttgcgcaacgttgttgccattgctacaggcatcgtggtgtcacgctcgtcgtttg
gtatggcttcattcagctccggttcccaacgatcaaggcgagttacatgatcccccatgttgtgcaaaaaagcggttagctccttcggt
cctccgatcgttgtcagaagtaagttggccgcagtgttatcactcatggttatggcagcactgcataattctcttactgtcatgccatcc
gtaagatgcttttctgtgactggtgagtactcaaccaagtcattctgagaatagtgtatgcggcgaccgagttgctcttgcccggcgtc
aatacgggataataccgcgccacatagcagaactttaaaagtgctcatcattggaaaacgttcttcggggcgaaaactctcaaggat
cttaccgctgttgagatccagttcgatgtaacccactcgtgcacccaactgatcttcagcatcttttactttcaccagcgtttctgggtga
gcaaaaacaggaaggcaaaatgccgcaaaaaagggaataagggcgacacggaaatgttgaatactcatactcttcctttttcaat
attattgaagcatttatcagggttattgtctcatgagcggatacatatttgaatgtatttagaaaaataaacaaataggggttccgcgc
acatttccccgaaaagtgccacctgacgtctaagaaaccattattatcatgacattaacctataaaaataggcgtatcacgaggccct
ttcgtctcgcgcgtttcggtgatgacggtgaaaacctctgacacatgcagctcccggagacggtcacagcttgtctgtaagcggatgc
cgggagcagacaagcccgtcagggcgcgtcagcgggtgttggcgggtgtcggggctggcttaactatgcggcatcagagcagatt
gtactgagagtgcaccatatgcggtgtgaaataccgcacagatgcgtaaggagaaaataccgcatcaggcgccattcgccattcag
gctgcgcaactgttgggaagggcgatcggtgcgggcctcttcgctattacgccagctggcgaaagggggatgtgctgcaaggcga
ttaagttgggtaacgccagggttttcccagtcacgacgttgtaaaacgacggccagtgaattc
SA1 sequence (SEQ ID NO: 60)
TTGCATCCAGACACCATCAA
SA2 sequence (SEQ ID NO: 61)
ATCCCCCCAGGTGTCTGCAG
SA3 sequence (SEQ ID NO: 62)
GTGTCTGCAGGCTCAAAGAG
Inactivated SA1 sequence (SEQ ID NO: 63)
TTGCATCCTGACACCATCAA
Inactivated SA2 sequence (SEQ ID NO: 64)
ATCCCCCCTGGTGTCTGCAG
Inactivated SA3 sequence (SEQ ID NO: 65)
GTGTCTGCIGGCTCAAAGAG
sh734 (SEQ ID NO: 66)
AGGATATGCCCTTGACTATTTGTCCGACATAGTCAAGGGCATATCC
sh734 with multi-t termination sequence (SEQ ID NO: 67)
AGGATATGCCCTTGACTATTTGTCCGACATAGTCAAGGGCATATCCTTTTTT
shRNA734 single t termination sequence (SEQ ID NO: 68)
AGGATATGCCCTTGACTATTTGTCCGACATAGTCAAGGGCATATCCT
7SK RNA promoter (SEQ ID NO: 69)
ATCGACGTGCAGTATTTAGCATGCCCCACCCATCTGCAAGGCATTCTGGATAGTGTCAAAACAGCCGG
AAATCAAGTCCGTTTATCTCAAACTTTAGCATTTTGGGAATAAATGATATTTGCTATGCTGGTTAAATTA
GATTTTAGTTAAATTTCCTGCTGAAGCTCTAGTACGATAAGTAACTTGACCTAAGTGTAAAGTTGAGAT
TTCCTTCAGGTTTATATAGCTTGTGCGCCGCCTGGGTACCTC
7SK RNA promoter (SEQ ID NO: 70)
ATCGACGTGCAGTCGGGCTACTGCCCCACCCATAGTACCGGCATTCTGGATAGTGTCAAAACAGCCG
GAAATCAAGTCCGTTTATCTCAAACTTTAGCATTTTGGGAATAAATGATATTTGCTATGCTGGTTAAAT
TAGATTTTAGTTAAATTTCCTGCTGAAGCTCTAGTACGATAAGTAACTTGACCTAAGTGTAAAGTTGAG
ATTTCCTTCAGGTTTATATAGCTTGTGCGCCGCCTGGGTACCTC
7SK RNA promoter (SEQ ID NO: 71)
CTGCAGTATTTAGCATGCCCCACCCATCTGCAAGGCATTCTGGATAGTGTCAAAACAGCCGGAAATCA
AGTCCGTTTATCTCAAACTTTAGCATTTTGGGAATAAATGATATTTGCTATGCTGGTTAAATTAGATTTT
AGTTAAATTTCCTGCTGAAGCTCTAGTACGATAAGCAACTTGACCTAAGTGTAAAGTTGAGATTTCCTT
CAGGTTTATATAGCTTGTGCGCCGCCTGGGTACCTC
MND promoter (SEQ ID NO: 72)
GAACAGAGAGACAGCAGAATATGGGCCAAACAGGATATCTGTGGTAAGCAGTTCCTGCCCCGGCTCA
GGGCCAAGAACAGTTGGAACAGCAGAATATGGGCCAAACAGGATATCTGTGGTAAGCAGTTCCTGCC
CCGGCTCAGGGCCAAGAACAGATGGTCCCCAGATGCGGTCCCGCCCTCAGCAGTTTCTAGAGAACCA
TCAGATGTTTCCAGGGTGCCCCAAGGACCTGAAATGACCCTGTGCCTTATTTGAACTAACCAATCAGT
TCGCTTCTCGCTTCTGTTCGCGCGCTTCTGCTCCCCGAGCTCTATATAAGCAGAGCTCGTTTAGTGAA
CCGTCAGATC
WASWT cDNA (wild-type ORF) (SEQ ID NO: 73)
ATGAGTGGGGGCCCAATGGGAGGAAGGCCCGGGGGCCGAGGAGCACCAGCGGTTCAGCAGAACAT
ACCCTCCACCCTCCTCCAGGACCACGAGAACCAGCGACTCTTTGAGATGCTTGGACGAAAATGCTTGA
CGCTGGCCACTGCAGTTGTTCAGCTGTACCTGGCGCTGCCCCCTGGAGCTGAGCACTGGACCAAGGA
GCATTGTGGGGCTGTGTGCTTCGTGAAGGATAACCCCCAGAAGTCCTACTTCATCCGCCTTTACGGCC
TTCAGGCTGGTCGGCTGCTCTGGGAACAGGAGCTGTACTCACAGCTTGTCTACTCCACCCCCACCCC
CTTCTTCCACACCTTCGCTGGAGATGACTGCCAAGCGGGGCTGAACTTTGCAGACGAGGACGAGGCC
CAGGCCTTCCGGGCACTCGTGCAGGAGAAGATACAAAAAAGGAATCAGAGGCAAAGTGGAGACAGA
CGCCAGCTACCCCCACCACCAACACCAGCCAATGAAGAGAGAAGAGGAGGGCTCCCACCCCTGCCC
CTGCATCCAGGTGGAGACCAAGGAGGCCCTCCAGTGGGTCCGCTCTCCCTGGGGCTGGCGACAGTG
GACATCCAGAACCCTGACATCACGAGTTCACGATACCGTGGGCTCCCAGCACCTGGACCTAGCCCAG
CTGATAAGAAACGCTCAGGGAAGAAGAAGATCAGCAAAGCTGATATTGGTGCACCCAGTGGATTCAA
GCATGTCAGCCACGTGGGGTGGGACCCCCAGAATGGATTTGACGTGAACAACCTCGACCCAGATCTG
CGGAGTCTGTTCTCCAGGGCAGGAATCAGCGAGGCCCAGCTCACCGACGCCGAGACCTCTAAACTTA
TCTACGACTTCATTGAGGACCAGGGTGGGCTGGAGGCTGTGCGGCAGGAGATGAGGCGCCAGGAGC
CACTTCCGCCGCCCCCACCGCCATCTCGAGGAGGGAACCAGCTCCCCCGGCCCCCTATTGTGGGGG
GTAACAAGGGTCGTTCTGGTCCACTGCCCCCTGTACCTTTGGGGATTGCCCCACCCCCACCAACACCC
CGGGGACCCCCACCCCCAGGCCGAGGGGGTCCTCCACCACCACCCCCTCCAGCTACTGGACGTTCT
GGACCACTGCCCCCTCCACCCCCTGGAGCTGGTGGGCCACCCATGCCACCACCACCGCCACCACCGC
CACCGCCGCCCAGCTCCGGGAATGGACCAGCCCCTCCCCCACTCCCTCCTGCTCTGGTGCCTGCCGG
GGGCCTGGCCCCTGGTGGGGGTCGGGGAGCGCTTTTGGATCAAATCCGGCAGGGAATTCAGCTGAA
CAAGACCCCTGGGGCCCCAGAGAGCTCAGCGCTGCAGCCACCACCTCAGAGCTCAGAGGGACTGGT
GGGGGCCCTGATGCACGTGATGCAGAAGAGAAGCAGAGCCATCCACTCCTCCGACGAAGGGGAGGA
CCAGGCTGGCGATGAAGATGAAGATGATGAATGGGATGAC
WASWT cDNA (Genbank accession no. AB590224.1) (SEQ ID NO: 74)
ATGAGTGGGGGCCCAATGGGAGGAAGGCCCGGGGGCCGAGGAGCACCAGCGGTTCAGCAGAACAT
ACCCTCCACCCTCCTCCAGGACCACGAGAACCAGCGACTCTTTGAGATGCTTGGACGAAAATGCTTGA
CGCTGGCCACTGCAGTTGTTCAGCTGTACCTGGCGCTGCCCCCTGGAGCTGAGCACTGGACCAAGGA
GCATTGTGGGGCTGTGTGCTTCGTGAAGGATAACCCCCAGAAGTCCTACTTCATCCGCCTTTACGGCC
TTCAGGCTGGTCGGCTGCTCTGGGAACAGGAGCTGTACTCACAGCTTGTCTACTCCACCCCCACCCC
CTTCTTCCACACCTTCGCTGGAGATGACTGCCAAGCGGGGCTGAACTTTGCAGACGAGGACGAGGCC
CAGGCCTTCCGGGCCCTCGTGCAGGAGAAGATACAAAAAAGGAATCAGAGGCAAAGTGGAGACAGA
CGCCAGCTACCCCCACCACCAACACCAGCCAATGAAGAGAGAAGAGGAGGGCTCCCACCCCTGCCC
CTGCATCCAGGTGGAGACCAAGGAGGCCCTCCAGTGGGTCCGCTCTCCCTGGGGCTGGCGACAGTG
GACATCCAGAACCCTGACATCACGAGTTCACGATACCGTGGGCTCCCAGCACCTGGACCTAGCCCAG
CTGATAAGAAACGCTCAGGGAAGAAGAAGATCAGCAAAGCTGATATTGGTGCACCCAGTGGATTCAA
GCATGTCAGCCACGTGGGGTGGGACCCCCAGAATGGATTTGACGTGAACAACCTCGACCCAGATCTG
CGGAGTCTGTTCTCCAGGGCAGGAATCAGCGAGGCCCAGCTCACCGACGCCGAGACCTCTAAACTTA
TCTACGACTTCATTGAGGACCAGGGTGGGCTGGAGGCTGTGCGGCAGGAGATGAGGCGCCAGGAGC
CACTTCCGCCGCCCCCACCGCCATCTCGAGGAGGGAACCAGCTCCCCCGGCCCCCTATTGTGGGGG
GTAACAAGGGTCGTTCTGGTCCACTGCCCCCTGTACCTTTGGGGATTGCCCCACCCCCACCAACACCC
CGGGGACCCCCACCCCCAGGCCGAGGGGGCCCTCCACCACCACCCCCTCCAGCTACTGGACGTTCT
GGACCACTGCCCCCTCCACCCCCTGGAGCTGGTGGGCCACCCATGCCACCACCACCGCCACCACCGC
CACCGCCGCCCAGCTCCGGGAATGGACCAGCCCCTCCCCCACTCCCTCCTGCTCTGGTGCCTGCCGG
GGGCCTGGCCCCTGGTGGGGGTCGGGGAGCGCTTTTGGATCAAATCCGGCAGGGAATTCAGCTGAA
CAAGACCCCTGGGGCCCCAGAGAGCTCAGCGCTGCAGCCACCACCTCAGAGCTCAGAGGGACTGGT
GGGGGCCCTGATGCACGTGATGCAGAAGAGAAGCAGAGCCATCCACTCCTCCGACGAAGGGGAGGA
CCAGGCTGGCGATGAAGATGAAGATGATGAATGGGATGAC
WASWT cDNA-codon optimized (SEQ ID NO: 75)
ATGTCTGGCGGACCTATGGGAGGTAGACCTGGTGGAAGAGGTGCTCCTGCCGTGCAGCAGAACATC
CCTTCTACACTGCTGCAGGACCACGAGAACCAGCGGCTGTTTGAGATGCTGGGCAGAAAGTGTCTGA
CCCTGGCTACAGCTGTGGTGCAGCTGTATCTGGCACTTCCTCCAGGCGCCGAGCACTGGACCAAAGA
ACATTGTGGCGCCGTGTGCTTCGTGAAGGACAACCCTCAGAAGTCCTACTTCATCCGGCTGTACGGA
CTGCAGGCTGGCAGACTGCTGTGGGAGCAAGAGCTGTACTCCCAGCTGGTGTACAGCACCCCTACAC
CTTTCTTCCACACCTTTGCCGGCGACGATTGTCAGGCCGGACTGAACTTTGCCGACGAGGATGAAGC
CCAGGCCTTCAGAGCACTGGTGCAAGAGAAGATCCAGAAGCGGAACCAGAGACAGAGCGGCGACAG
AAGGCAACTGCCTCCTCCACCTACACCAGCCAACGAGGAAAGAAGAGGCGGACTGCCTCCACTGCCT
CTTCATCCTGGCGGAGATCAAGGTGGACCTCCTGTGGGACCACTGTCTCTTGGACTGGCCACCGTGG
ACATTCAGAACCCCGATATCACCAGCAGCCGGTACAGAGGACTTCCCGCTCCTGGACCATCTCCTGC
CGACAAGAAGAGATCCGGGAAGAAGAAGATCAGCAAGGCCGACATCGGAGCCCCTAGCGGCTTTAA
ACACGTGTCCCACGTTGGATGGGACCCACAGAACGGCTTCGACGTGAACAATCTGGACCCCGACCTG
CGGAGCCTGTTTTCTAGAGCCGGAATCTCTGAGGCCCAGCTGACCGATGCCGAGACAAGCAAGCTGA
TCTACGACTTCATCGAGGACCAAGGCGGCCTGGAAGCCGTGCGACAAGAGATGAGAAGGCAAGAGC
CTCTGCCACCACCTCCACCTCCATCTAGAGGCGGAAACCAGCTGCCTAGACCTCCTATCGTTGGCGG
CAACAAGGGAAGATCTGGCCCTCTGCCTCCTGTGCCTCTGGGAATTGCTCCACCACCACCAACACCTA
GAGGCCCGCCTCCACCAGGCAGAGGTGGTCCTCCGCCGCCACCTCCTCCAGCAACAGGCAGATCTG
GACCACTTCCTCCTCCACCACCTGGTGCTGGTGGACCTCCAATGCCACCGCCACCGCCTCCGCCACC
TCCGCCTCCAAGTTCTGGAAATGGACCTGCTCCTCCTCCTTTGCCTCCTGCTTTGGTTCCTGCTGGCG
GATTGGCTCCAGGCGGAGGAAGAGGCGCACTCCTGGATCAGATCAGACAGGGCATCCAGCTGAACA
AGACCCCTGGCGCTCCTGAGAGTTCTGCTCTGCAACCGCCACCACAGTCTAGCGAAGGACTTGTGGG
AGCCCTGATGCACGTGATGCAGAAGAGAAGCAGAGCCATCCACAGCAGCGACGAAGGCGAAGATCA
AGCTGGCGACGAAGATGAGGACGACGAGTGGGACGAT
WASP (SEQ ID NO: 76)
MSGGPMGGRPGGRGAPAVQQNIPSTLLQDHENQRLFEMLGRKCLTLATAVVQLYLALPPGAEHWTKEH
CGAVCFVKDNPQKSYFIRLYGLQAGRLLWEQELYSQLVYSTPTPFFHTFAGDDCQAGLNFADEDEAQAFR
ALVQEKIQKRNQRQSGDRRQLPPPPTPANEERRGGLPPLPLHPGGDQGGPPVGPLSLGLATVDIQNPDIT
SSRYRGLPAPGPSPADKKRSGKKKISKADIGAPSGFKHVSHVGWDPQNGFDVNNLDPDLRSLFSRAGIS
EAQLTDAETSKLIYDFIEDQGGLEAVRQEMRRQEPLPPPPPPSRGGNQLPRPPIVGGNKGRSGPLPPVPLG
IAPPPPTPRGPPPPGRGGPPPPPPPATGRSGPLPPPPPGAGGPPMPPPPPPPPPPPSSGNGPAPPPLPPALVP
AGGLAPGGGRGALLDQIRQGIQLNKTPGAPESSALQPPPQSSEGLVGALMHVMQKRSRAIHSSDEGED
QAGDEDEDDEWDD
WPRE mut6 (SEQ ID NO: 77)
AATCAACCTCTGGATTACAAAATTTGTGAAAGATTGACTGGTATTCTTAACTATGTTGCTCCTTTTACG
CTATGTGGATACGCTGCTTTAATGCCTTTGTATCATGCTATTGCTTCCCGTATGGCTTTCATTTTCTCCT
CCTTGTATAAATCCTGGTTGCTGTCTCTTTATGAGGAGTTGTGGCCCGTTGTCAGGCAACGTGGCGTG
GTGTGCACTGTGTTTGCTGACGCAACCCCCACTGGTTGGGGCATTGCCACCACCTGTCAGCTCCTTTC
CGGGACTTTCGCTTTCCCCCTCCCTATTGCCACGGCGGAACTCATCGCCGCCTGCCTTGCCCGCTGCT
GGACAGGGGCTCGGCTGTTGGGCACTGACAATTCCGTGGTGTTGTCGGGGAAATCATCGTCCTTTCC
TTGGCTGCTCGCCTGTGTTGCCACCTGGATTCTGCGCGGGACGTCCTTCTGCTACGTCCCTTCGGCC
CTCAATCCAGCGGACCTTCCTTCCCGCGGCCTGCTGCCGGCTCTGCGGCCTCTTCCGCGTCTTCGCC
TTCGCCCTCAGACGAGTCGGATCTCCCTTTGGGCCGCCTCCCCGCA
WPRE mut7 (SEQ ID NO: 78)
AATCAACCTCTGGATTACAAAATTTGTGAAAGATTGACTGGTATTCTTAACTATGTTGCTCCTTTTACG
CTATGTGGATACGCTGCTTTAATGCCTTTGTATCATGCTATTGCTTCCCGTATGGCTTTCATTTTCTCCT
CCTTGTATAAATCCTGGTTGCTGTCTCTTTATGAGGAGTTGTGGCCCGTTGTCAGGCAACGTGGCGTG
GTGTGCACTGTGTTTGCTGACGCAACCCCCACTGGTTGGGGCATTGCCACCACCTGTCAGCTCCTTTC
CGGGACTTTCGCTTTCCCCCTCCCTATTGCCACGGCGGAACTCATCGCCGCCTGCCTTGCCCGCTGCT
GGACAGGGGCTCGGCTGTTGGGCACTGACAATTCCGTGGTGTTGTCGGGGAAATCATCGTCCTTTCC
TTGGCTGTTCGCCTGTGTTGCCACCTGGATTCTGCGCGGGACGTCCTTCTGCTACGTCCCTTCGGCCC
TCAATCCAGCGGACCTTCCTTCCCGCGGCCTGCTGCCGGCTCTGCGGCCTCTTCCGCGTCTTCGCCTT
CGCCCTCAGACGAGTCGGATCTCCCTTTGGGCCGCCTCCCCGCA
7tet operator (SEQ ID NO: 79)
TTTACCACTCCCTATCAGTGATAGAGAAAAGTGAAAGTCGAGTTTACCACTCCCTATCAGTGATAGAG
AAAAGTGAAAGTCGAGTTTACCACTCCCTATCAGTGATAGAGAAAAGTGAAAGTCGAGTTTACCACTC
CCTATCAGTGATAGAGAAAAGTGAAAGTCGAGTTTACCAGTCCCTATCAGTGATAGAGAAAAGTGAAA
GTCGAGTTTACCACTCCCTATCAGTGATAGAGAAAAGTGAAAGTCGAGTTTACCACTCCCTATCAGTG
ATAGAGAAAAGTGAAA
β-globin poly(A) signal (SEQ ID NO: 80)
GATCTTTTTCCCTCTGCCAAAAATTATGGGGACATCATGAAGCCCCTTGAGCATCTGACTTCTGGCTAA
TAAAGGAAATTTATTTTCATTGCAATAGTGTGTTGGAATTTTTTGTGTCTCTCACTCGGAAGGACATAT
GGGAGGGCAAATCATTTAAAACATCAGAATGAGTATTTGGTTTAGAGTTTGGCAACATATGCCCATAT
GCTGGCTGCCATGAACAAAGGTTGGCTATAAAGAGGTCATCAGTATATGAAACAGCCCCCTGCTGTC
CATTCCTTATTCCATAGAAAAGCCTTGACTTGAGGTTAGATTTTTTTTATATTTTGTTTTGTGTTATTTTT
TTCTTTAACATCCCTAAAATTTTCCTTACATGTTTTACTAGCCAGATTTTTCCTCCTCTCCTGACTACTCC
CAGTCATAGCTGTCCCTCTTCTCTTATGGAGATC
pBRNGTR83 pTL20c MND WAS 650 (SEQ ID NO: 81)
ggccgcctcggccaaacagcccttgagtttaccactccctatcagtgatagagaaaagtgaaagtcgagtttaccactccctatcagt
gatagagaaaagtgaaagtcgagtttaccactccctatcagtgatagagaaaagtgaaagtcgagtttaccactccctatcagtgat
agagaaaagtgaaagtcgagtttaccagtccctatcagtgatagagaaaagtgaaagtcgagtttaccactccctatcagtgatag
agaaaagtgaaagtcgagtttaccactccctatcagtgatagagaaaagtgaaagtcgagctcgccatgggaggcgtggcctggg
cgggactggggagtggcgagccctcagatcctgcatataagcagctgctttttgcctgtactgggtctctctggttagaccagatctg
agcctgggagctctctggctaactagggaacccactgcttaagcctcaataaagcttgccttgagtgcttcaagtagtgtgtgcccgt
ctgttgtgtgactctggtaactagagatccctcagacccttttagtcagtgtggaaaatctctagcagtggcgcccgaacagggactt
gaaagcgaaagggaaaccagaggagctctctcgacgcaggactcggcttgctgaagcgcgcacggcaagaggcgaggggcgg
cgactggtgagtacgccaaaaattttgactagcggaggctagaaggagagagatgggtgcgagagcgtcagtattaagcggggg
agaattagatcgcgatgggaaaaaattcggttaaggccagggggaaagaaaaaatataaattaaaacatatagtatgggcaagc
agggagctagaacgattcgcagttaatactggcctgttagaaacatcagaaggctgtagacaaatactgggacagctacaaccatc
ccttcagacaggatcagaagaacttagatcattatataatacagtagcaaccctctattgtgtgcatcaaaggatagagataaaaga
caccaaggaagctttagacaagatagaggaagagcaaaaaaaagtaagaaaaaagcacagcaagcagcaggatcttcagac
ctggaaattccctacaatccccaaagtcaaggagtagtagaatctatgaataaagaattaaagaaaattataggacaggtaagag
atcaggctgaacatcttaagacagcagtacaaatggcagtattcatccacaattttaaaagaaaaggggggattggggggtacagt
gcaggggaaagaatagtagacataatagcaacagacatacaaactaaagaattacaaaaacaaattacaaaaattcaaaattttc
gggtttattacagggacagcagaaatccactttggaaaggaccagcaaagctcctctggaaaggtgaaggggcagtagtaataca
agataatagtgacataaaagtagtgccaagaagaaaagcaaagatcattagggattatggaaaacagatggcaggtgatgattg
tgtggcaagtagacaggatgaggattagaacatggaaaagtttagtaaaacaccataaggaggagatatgagggacaattggag
aagtgaattatataaatataaagtagtaaaaattgaaccattaggagtagcacccaccaaggcaaagagaagagtggtgcagag
agaaaaaagagcagtgggaataggagctttgttccttgggttcttgggagcagcaggaagcactatgggcgcagcgtcaatgacg
ctgacggtacaggccagacaattattgtctggtatagtgcagcagcagaacaatttgctgagggctattgaggcgcaacagcatct
gttgcaactcacagtctggggcatcaagcagctccaggcaagaatcctggctgtggaaagatacctaaaggatcaacagctcctgg
ggatttggggttgctctggaaaactcatttgcaccactgctgtgccttggaatgctagttggagtaataaatctctggaacagatttgg
aatcacacgacctggatggagtgggacagagaaattaacaattacacaagcttaatacactccttaattgaagaatcgcaaaacca
gcaagaaaagaatgaacaagaattattggaattagataaatgggcaagtttgtggaattggtttaacataacaaattggctgtggt
atataaaattattcataatgatagtaggaggcttggtaggtttaagaatagtttttgctgtactttctatagtgaatagagttaggcag
ggatattcaccattatcgtttcagacccacctcccaaccccgaggggaccgagctcaagcttcgaagcgatcgcacgcgtggatccg
aacagagagacagcagaatatgggccaaacaggatatctgtggtaagcagttcctgccccggctcagggccaagaacagttgga
acagcagaatatgggccaaacaggatatctgtggtaagcagttcctgccccggctcagggccaagaacagatggtccccagatgc
ggtcccgccctcagcagtttctagagaaccatcagatgtttccagggtgccccaaggacctgaaatgaccctgtgccttatttgaact
aaccaatcagttcgcttctcgcttctgttcgcgcgcttctgctccccgagctctatataagcagagctcgtttagtgaaccgtcagatcg
gcgcgccaattcaagcgagaagacaagggcagccgccaccatgagtgggggcccaatgggaggaaggcccgggggccgagga
gcaccagcggttcagcagaacataccctccaccctcctccaggaccacgagaaccagcgactctttgagatgcttggacgaaaatg
cttgacgctggccactgcagttgttcagctgtacctggcgctgccccctggagctgagcactggaccaaggagcattgtggggctgt
gtgcttcgtgaaggataacccccagaagtcctacttcatccgcctttacggccttcaggctggtcggctgctctgggaacaggagctg
tactcacagcttgtctactccacccccacccccttcttccacaccttcgctggagatgactgccaagcggggctgaactttgcagacga
ggacgaggcccaggccttccgggcactcgtgcaggagaagatacaaaaaaggaatcagaggcaaagtggagacagacgccag
ctacccccaccaccaacaccagccaatgaagagagaagaggagggctcccacccctgcccctgcatccaggtggagaccaagga
ggccctccagtgggtccgctctccctggggctggcgacagtggacatccagaaccctgacatcacgagttcacgataccgtgggctc
ccagcacctggacctagcccagctgataagaaacgctcagggaagaagaagatcagcaaagctgatattggtgcacccagtggat
tcaagcatgtcagccacgtggggtgggacccccagaatggatttgacgtgaacaacctcgacccagatctgcggagtctgttctcca
gggcaggaatcagcgaggcccagctcaccgacgccgagacctctaaacttatctacgacttcattgaggaccagggtgggctgga
ggctgtgcggcaggagatgaggcgccaggagccacttccgccgcccccaccgccatctcgaggagggaaccagctcccccggccc
cctattgtggggggtaacaagggtcgttctggtccactgccccctgtacctttggggattgccccacccccaccaacaccccggggac
ccccacccccaggccgagggggtcctccaccaccaccccctccagctactggacgttctggaccactgccccctccaccccctggag
ctggtgggccacccatgccaccaccaccgccaccaccgccaccgccgcccagctccgggaatggaccagcccctcccccactccctc
ctgctctggtgcctgccgggggcctggcccctggtgggggtcggggagcgcttttggatcaaatccggcagggaattcagctgaac
aagacccctggggccccagagagctcagcgctgcagccaccacctcagagctcagagggactggtgggggccctgatgcacgtg
atgcagaagagaagcagagccatccactcctccgacgaaggggaggaccaggctggcgatgaagatgaagatgatgaatggga
tgactgataactagtaatcaacctctggattacaaaatttgtgaaagattgactggtattcttaactatgttgctccttttacgctatgtg
gatacgctgctttaatgcctttgtatcatgctattgcttcccgtatggctttcattttctcctccttgtataaatcctggttgctgtctcttta
tgaggagttgtggcccgttgtcaggcaacgtggcgtggtgtgcactgtgtttgctgacgcaacccccactggttggggcattgccacc
acctgtcagctcctttccgggactttcgctttccccctccctattgccacggcggaactcatcgccgcctgccttgcccgctgctggaca
ggggctcggctgttgggcactgacaattccgtggtgttgtcggggaaatcatcgtcctttccttggctgctcgcctgtgttgccacctgg
attctgcgcgggacgtccttctgctacgtcccttcggccctcaatccagcggaccttccttcccgcggcctgctgccggctctgcggcct
cttccgcgtcttcgccttcgccctcagacgagtcggatctccctttgggccgcctccccgcacgtacgaccggtgcggccgcatcgatg
ccgtagtacctttaagaccaatgacttacaaggcagctgtagatcttagccactttttaaaagaaaaggggggactggaagggcta
attcactcccaaagaagacaagatagccccatcctcactgactccgtcctggagttggatgagagataatggccttacgttgtgcca
ggggagggtcgggctggatttagcaagatttaccttctccaaagagcggtgctgcagtggcacagctgcccacggaggtgggggg
gtcaccgtccctggaggtgatgaagaactgtggggatgtggcactgagggacatggccagtgggcacggtgggtgggttggggtt
ggtcttggggatcttggagggcttttccagccttcatgatttgacgattgtatgaacatctacatggcaattctccagctgcctgtccca
gtcctactgacccagctgtatctctccaggcaagctcttccaccccttctgcttgcatccagacaccatcaaacatgcaggctcagaca
catgatatcaagctttttccccgtatccccccaggtgtctgcaggctcaaagagcagcgagaagcgttcagaggaaagcgatcccgt
gccaccttccccgtgcccgggctgtccccgcacgctgccggctcggggatgcggggggagcgccggaccggagcggagccccgg
gcggctcgctgctgccccctagcgggggagggacgtaattacatccctgggggctttgggggggggctgtccccgtgagctcccca
gatctgctttttgcctgtactgggtctctctggttagaccagatctgagcctgggagctctctggctaactagggaacccactgcttaag
cctcaataaagcttcagctgctcgagctagcagatctttttccctctgccaaaaattatggggacatcatgaagccccttgagcatctg
acttctggctaataaaggaaatttattttcattgcaatagtgtgttggaattttttgtgtctctcactcggaaggacatatgggagggca
aatcatttaaaacatcagaatgagtatttggtttagagtttggcaacatatgcccatatgctggctgccatgaacaaaggttggctata
aagaggtcatcagtatatgaaacagccccctgctgtccattccttattccatagaaaagccttgacttgaggttagattttttttatatttt
gttttgtgttatttttttctttaacatccctaaaattttccttacatgttttactagccagatttttcctcctctcctgactactcccagtcat
agctgtccctcttctcttatggagatccctcgacctgcagcccaagcttggcgtaatcatggtcatagctgtttcctgtgtgaaattgttatcc
gctcacaattccacacaacatacgagccggaagcataaagtgtaaagcctggggtgcctaatgagtgagctaactcacattaattg
cgttgcgctcactgcccgctttccagtcgggaaacctgtcgtgccagcggatccgcatctcaattagtcagcaaccatagtcccgccc
ctaactccgcccatcccgcccctaactccgcccagttccgcccattctccgccccatggctgactaattttttttatttatgcagaggccg
aggccgcctcggcctctgagctattccagaagtagtgaggaggcttttttggaggcctaggcttttgcaaaaagctgtcgactgcag
aggcctgcatgcaagcttggcgtaatcatggtcatagctgtttcctgtgtgaaattgttatccgctcacaattccacacaacatacgag
ccggaagcataaagtgtaaagcctggggtgcctaatgagtgagctaactcacattaattgcgttgcgctcactgcccgctttccagtc
gggaaacctgtcgtgccagctgcattaatgaatcggccaacgcgcggggagaggcggtttgcgtattgggcgctcttccgcttcctc
gctcactgactcgctgcgctcggtcgttcggctgcggcgagcggtatcagctcactcaaaggcggtaatacggttatccacagaatc
aggggataacgcaggaaagaacatgtgagcaaaaggccagcaaaaggccaggaaccgtaaaaaggccgcgttgctggcgtttt
tccataggctccgcccccctgacgagcatcacaaaaatcgacgctcaagtcagaggtggcgaaacccgacaggactataaagata
ccaggcgtttccccctggaagctccctcgtgcgctctcctgttccgaccctgccgcttaccggatacctgtccgcctttctcccttcggga
agcgtggcgctttctcatagctcacgctgtaggtatctcagttcggtgtaggtcgttcgctccaagctgggctgtgtgcacgaaccccc
cgttcagcccgaccgctgcgccttatccggtaactatcgtcttgagtccaacccggtaagacacgacttatcgccactggcagcagcc
actggtaacaggattagcagagcgaggtatgtaggcggtgctacagagttcttgaagtggtggcctaactacggctacactagaag
aacagtatttggtatctgcgctctgctgaagccagttaccttcggaaaaagagttggtagctcttgatccggcaaacaaaccaccgct
ggtagcggtggtttttttgtttgcaagcagcagattacgcgcagaaaaaaaggatctcaagaagatcctttgatcttttctacggggtc
tgacgctcagtggaacgaaaactcacgttaagggattttggtcatgagattatcaaaaaggatcttcacctagatccttttaaattaa
aaatgaagttttaaatcaatctaaagtatatatgagtaaacttggtctgacagttaccaatgcttaatcagtgaggcacctatctcagc
gatctgtctatttcgttcatccatagttgcctgactccccgtcgtgtagataactacgatacgggagggcttaccatctggccccagtgc
tgcaatgataccgcgagacccacgctcaccggctccagatttatcagcaataaaccagccagccggaagggccgagcgcagaagt
ggtcctgcaactttatccgcctccatccagtctattaattgttgccgggaagctagagtaagtagttcgccagttaatagtttgcgcaac
gttgttgccattgctacaggcatcgtggtgtcacgctcgtcgtttggtatggcttcattcagctccggttcccaacgatcaaggcgagtt
acatgatcccccatgttgtgcaaaaaagcggttagctccttcggtcctccgatcgttgtcagaagtaagttggccgcagtgttatcact
catggttatggcagcactgcataattctcttactgtcatgccatccgtaagatgcttttctgtgactggtgagtactcaaccaagtcattc
tgagaatagtgtatgcggcgaccgagttgctcttgcccggcgtcaatacgggataataccgcgccacatagcagaactttaaaagt
gctcatcattggaaaacgttcttcggggcgaaaactctcaaggatcttaccgctgttgagatccagttcgatgtaacccactcgtgcac
ccaactgatcttcagcatcttttactttcaccagcgtttctgggtgagcaaaaacaggaaggcaaaatgccgcaaaaaagggaataa
gggcgacacggaaatgttgaatactcatactcttcctttttcaatattattgaagcatttatcagggttattgtctcatgagcggatacat
atttgaatgtatttagaaaaataaacaaataggggttccgcgcacatttccccgaaaagtgccacctgacgtctaagaaaccattatt
atcatgacattaacctataaaaataggcgtatcacgaggccctttcgtctcgcgcgtttcggtgatgacggtgaaaacctctgacaca
tgcagctcccggagacggtcacagcttgtctgtaagcggatgccgggagcagacaagcccgtcagggcgcgtcagcgggtgttgg
cgggtgtcggggctggcttaactatgcggcatcagagcagattgtactgagagtgcaccatatgcggtgtgaaataccgcacagat
gcgtaaggagaaaataccgcatcaggcgccattcgccattcaggctgcgcaactgttgggaagggcgatcggtgcgggcctcttcg
ctattacgccagctggcgaaagggggatgtgctgcaaggcgattaagttgggtaacgccagggttttcccagtcacgacgttgtaa
aacgacggccagtgaattc
pBRNGTR87 pTL20c MND WAS 650 SAmut (SEQ ID NO: 82)
ggccgcctcggccaaacagcccttgagtttaccactccctatcagtgatagagaaaagtgaaagtcgagtttaccactccctatcagt
gatagagaaaagtgaaagtcgagtttaccactccctatcagtgatagagaaaagtgaaagtcgagtttaccactccctatcagtgat
agagaaaagtgaaagtcgagtttaccagtccctatcagtgatagagaaaagtgaaagtcgagtttaccactccctatcagtgatag
agaaaagtgaaagtcgagtttaccactccctatcagtgatagagaaaagtgaaagtcgagctcgccatgggaggcgtggcctggg
cgggactggggagtggcgagccctcagatcctgcatataagcagctgctttttgcctgtactgggtctctctggttagaccagatctg
agcctgggagctctctggctaactagggaacccactgcttaagcctcaataaagcttgccttgagtgcttcaagtagtgtgtgcccgt
ctgttgtgtgactctggtaactagagatccctcagacccttttagtcagtgtggaaaatctctagcagtggcgcccgaacagggactt
gaaagcgaaagggaaaccagaggagctctctcgacgcaggactcggcttgctgaagcgcgcacggcaagaggcgaggggcgg
cgactggtgagtacgccaaaaattttgactagcggaggctagaaggagagagatgggtgcgagagcgtcagtattaagcggggg
agaattagatcgcgatgggaaaaaattcggttaaggccagggggaaagaaaaaatataaattaaaacatatagtatgggcaagc
agggagctagaacgattcgcagttaatactggcctgttagaaacatcagaaggctgtagacaaatactgggacagctacaaccatc
ccttcagacaggatcagaagaacttagatcattatataatacagtagcaaccctctattgtgtgcatcaaaggatagagataaaaga
caccaaggaagctttagacaagatagaggaagagcaaaacaaaagtaagaaaaaagcacagcaagcagcaggatcttcagac
ctggaaattccctacaatccccaaagtcaaggagtagtagaatctatgaataaagaattaaagaaaattataggacaggtaagag
atcaggctgaacatcttaagacagcagtacaaatggcagtattcatccacaattttaaaagaaaaggggggattggggggtacagt
gcaggggaaagaatagtagacataatagcaacagacatacaaactaaagaattacaaaaacaaattacaaaaattcaaaattttc
gggtttattacagggacagcagaaatccactttggaaaggaccagcaaagctcctctggaaaggtgaaggggcagtagtaataca
agataatagtgacataaaagtagtgccaagaagaaaagcaaagatcattagggattatggaaaacagatggcaggtgatgattg
tgtggcaagtagacaggatgaggattagaacatggaaaagtttagtaaaacaccataaggaggagatatgagggacaattggag
aagtgaattatataaatataaagtagtaaaaattgaaccattaggagtagcacccaccaaggcaaagagaagagtggtgcagag
agaaaaaagagcagtgggaataggagctttgttccttgggttcttgggagcagcaggaagcactatgggcgcagcgtcaatgacg
ctgacggtacaggccagacaattattgtctggtatagtgcagcagcagaacaatttgctgagggctattgaggcgcaacagcatct
gttgcaactcacagtctggggcatcaagcagctccaggcaagaatcctggctgtggaaagatacctaaaggatcaacagctcctgg
ggatttggggttgctctggaaaactcatttgcaccactgctgtgccttggaatgctagttggagtaataaatctctggaacagatttgg
aatcacacgacctggatggagtgggacagagaaattaacaattacacaagcttaatacactccttaattgaagaatcgcaaaacca
gcaagaaaagaatgaacaagaattattggaattagataaatgggcaagtttgtggaattggtttaacataacaaattggctgtggt
atataaaattattcataatgatagtaggaggcttggtaggtttaagaatagtttttgctgtactttctatagtgaatagagttaggcag
ggatattcaccattatcgtttcagacccacctcccaaccccgaggggaccgagctcaagcttcgaagcgatcgcacgcgtggatccg
aacagagagacagcagaatatgggccaaacaggatatctgtggtaagcagttcctgccccggctcagggccaagaacagttgga
acagcagaatatgggccaaacaggatatctgtggtaagcagttcctgccccggctcagggccaagaacagatggtccccagatgc
ggtcccgccctcagcagtttctagagaaccatcagatgtttccagggtgccccaaggacctgaaatgaccctgtgccttatttgaact
aaccaatcagttcgcttctcgcttctgttcgcgcgcttctgctccccgagctctatataagcagagctcgtttagtgaaccgtcagatcg
gcgcgccaattcaagcgagaagacaagggcagccgccaccatgagtgggggcccaatgggaggaaggcccgggggccgagga
gcaccagcggttcagcagaacataccctccaccctcctccaggaccacgagaaccagcgactctttgagatgcttggacgaaaatg
cttgacgctggccactgcagttgttcagctgtacctggcgctgccccctggagctgagcactggaccaaggagcattgtggggctgt
gtgcttcgtgaaggataacccccagaagtcctacttcatccgcctttacggccttcaggctggtcggctgctctgggaacaggagctg
tactcacagcttgtctactccacccccacccccttcttccacaccttcgctggagatgactgccaagcggggctgaactttgcagacga
ggacgaggcccaggccttccgggcactcgtgcaggagaagatacaaaaaaggaatcagaggcaaagtggagacagacgccag
ctacccccaccaccaacaccagccaatgaagagagaagaggagggctcccacccctgcccctgcatccaggtggagaccaagga
ggccctccagtgggtccgctctccctggggctggcgacagtggacatccagaaccctgacatcacgagttcacgataccgtgggctc
ccagcacctggacctagcccagctgataagaaacgctcagggaagaagaagatcagcaaagctgatattggtgcacccagtggat
tcaagcatgtcagccacgtggggtgggacccccagaatggatttgacgtgaacaacctcgacccagatctgcggagtctgttctcca
gggcaggaatcagcgaggcccagctcaccgacgccgagacctctaaacttatctacgacttcattgaggaccagggtgggctgga
ggctgtgcggcaggagatgaggcgccaggagccacttccgccgcccccaccgccatctcgaggagggaaccagctcccccggccc
cctattgtggggggtaacaagggtcgttctggtccactgccccctgtacctttggggattgccccacccccaccaacaccccggggac
ccccacccccaggccgagggggtcctccaccaccaccccctccagctactggacgttctggaccactgccccctccaccccctggag
ctggtgggccacccatgccaccaccaccgccaccaccgccaccgccgcccagctccgggaatggaccagcccctcccccactccctc
ctgctctggtgcctgccgggggcctggcccctggtgggggtcggggagcgcttttggatcaaatccggcagggaattcagctgaac
aagacccctggggccccagagagctcagcgctgcagccaccacctcagagctcagagggactggtgggggccctgatgcacgtg
atgcagaagagaagcagagccatccactcctccgacgaaggggaggaccaggctggcgatgaagatgaagatgatgaatggga
tgactgataactagtaatcaacctctggattacaaaatttgtgaaagattgactggtattcttaactatgttgctccttttacgctatgtg
gatacgctgctttaatgcctttgtatcatgctattgcttcccgtatggctttcattttctcctccttgtataaatcctggttgctgtctcttt
atgaggagttgtggcccgttgtcaggcaacgtggcgtggtgtgcactgtgtttgctgacgcaacccccactggttggggcattgccacc
acctgtcagctcctttccgggactttcgctttccccctccctattgccacggcggaactcatcgccgcctgccttgcccgctgctggaca
ggggctcggctgttgggcactgacaattccgtggtgttgtcggggaaatcatcgtcctttccttggctgctcgcctgtgttgccacctgg
attctgcgcgggacgtccttctgctacgtcccttcggccctcaatccagcggaccttccttcccgcggcctgctgccggctctgcggcct
cttccgcgtcttcgccttcgccctcagacgagtcggatctccctttgggccgcctccccgcacgtacgaccggtgcggccgcatcgatg
ccgtagtacctttaagaccaatgacttacaaggcagctgtagatcttagccactttttaaaagaaaaggggggactggaagggcta
attcactcccaaagaagacaagatagccccatcctcactgactccgtcctggagttggatgagagataatggccttacgttgtgcca
ggggagggtcgggctggatttagcaagatttaccttctccaaagagcggtgctgcagtggcacagctgcccacggaggtgggggg
gtcaccgtccctggaggtgatgaagaactgtggggatgtggcactgagggacatggccagtgggcacggtgggtgggttggggtt
ggtcttggggatcttggagggcttttccagccttcatgatttgacgattgtatgaacatctacatggcaattctccagctgcctgtccca
gtcctactgacccagctgtatctctccaggcaagctcttccaccccttctgcttgcatcctgacaccatcaaacatgcaggctcagacac
atgatatcaagctttttccccgtatcccccctggtgtctgcaggctcaaagagcagcgagaagcgttcagaggaaagcgatcccgtg
ccaccttccccgtgcccgggctgtccccgcacgctgccggctcggggatgcggggggagcgccggaccggagcggagccccggg
cggctcgctgctgccccctagcgggggagggacgtaattacatccctgggggctttgggggggggctgtccccgtgagctccccag
atctgctttttgcctgtactgggtctctctggttagaccagatctgagcctgggagctctctggctaactagggaacccactgcttaagc
ctcaataaagcttcagctgctcgagctagcagatctttttccctctgccaaaaattatggggacatcatgaagccccttgagcatctga
cttctggctaataaaggaaatttattttcattgcaatagtgtgttggaattttttgtgtctctcactcggaaggacatatgggagggcaa
atcatttaaaacatcagaatgagtatttggtttagagtttggcaacatatgcccatatgctggctgccatgaacaaaggttggctataa
agaggtcatcagtatatgaaacagccccctgctgtccattccttattccatagaaaagccttgacttgaggttagattttttttatattttg
ttttgtgttatttttttctttaacatccctaaaattttccttacatgttttactagccagatttttcctcctctcctgactactcccagtcat
agctgtccctcttctcttatggagatccctcgacctgcagcccaagcttggcgtaatcatggtcatagctgtttcctgtgtgaaattgttatc
cgctcacaattccacacaacatacgagccggaagcataaagtgtaaagcctggggtgcctaatgagtgagctaactcacattaattgc
gttgcgctcactgcccgctttccagtcgggaaacctgtcgtgccagcggatccgcatctcaattagtcagcaaccatagtcccgcccct
aactccgcccatcccgcccctaactccgcccagttccgcccattctccgccccatggctgactaattttttttatttatgcagaggccgag
gccgcctcggcctctgagctattccagaagtagtgaggaggcttttttggaggcctaggcttttgcaaaaagctgtcgactgcagag
gcctgcatgcaagcttggcgtaatcatggtcatagctgtttcctgtgtgaaattgttatccgctcacaattccacacaacatacgagcc
ggaagcataaagtgtaaagcctggggtgcctaatgagtgagctaactcacattaattgcgttgcgctcactgcccgctttccagtcgg
gaaacctgtcgtgccagctgcattaatgaatcggccaacgcgcggggagaggcggtttgcgtattgggcgctcttccgcttcctcgct
cactgactcgctgcgctcggtcgttcggctgcggcgagcggtatcagctcactcaaaggcggtaatacggttatccacagaatcagg
ggataacgcaggaaagaacatgtgagcaaaaggccagcaaaaggccaggaaccgtaaaaaggccgcgttgctggcgtttttcca
taggctccgcccccctgacgagcatcacaaaaatcgacgctcaagtcagaggtggcgaaacccgacaggactataaagataccag
gcgtttccccctggaagctccctcgtgcgctctcctgttccgaccctgccgcttaccggatacctgtccgcctttctcccttcgggaagcg
tggcgctttctcatagctcacgctgtaggtatctcagttcggtgtaggtcgttcgctccaagctgggctgtgtgcacgaaccccccgttc
agcccgaccgctgcgccttatccggtaactatcgtcttgagtccaacccggtaagacacgacttatcgccactggcagcagccactg
gtaacaggattagcagagcgaggtatgtaggcggtgctacagagttcttgaagtggtggcctaactacggctacactagaagaac
agtatttggtatctgcgctctgctgaagccagttaccttcggaaaaagagttggtagctcttgatccggcaaacaaaccaccgctggt
agcggtggtttttttgtttgcaagcagcagattacgcgcagaaaaaaaggatctcaagaagatcctttgatcttttctacggggtctga
cgctcagtggaacgaaaactcacgttaagggattttggtcatgagattatcaaaaaggatcttcacctagatccttttaaattaaaaa
tgaagttttaaatcaatctaaagtatatatgagtaaacttggtctgacagttaccaatgcttaatcagtgaggcacctatctcagcgat
ctgtctatttcgttcatccatagttgcctgactccccgtcgtgtagataactacgatacgggagggcttaccatctggccccagtgctgc
aatgataccgcgagacccacgctcaccggctccagatttatcagcaataaaccagccagccggaagggccgagcgcagaagtggt
cctgcaactttatccgcctccatccagtctattaattgttgccgggaagctagagtaagtagttcgccagttaatagtttgcgcaacgtt
gttgccattgctacaggcatcgtggtgtcacgctcgtcgtttggtatggcttcattcagctccggttcccaacgatcaaggcgagttac
atgatcccccatgttgtgcaaaaaagcggttagctccttcggtcctccgatcgttgtcagaagtaagttggccgcagtgttatcactca
tggttatggcagcactgcataattctcttactgtcatgccatccgtaagatgcttttctgtgactggtgagtactcaaccaagtcattctg
agaatagtgtatgcggcgaccgagttgctcttgcccggcgtcaatacgggataataccgcgccacatagcagaactttaaaagtgct
catcattggaaaacgttcttcggggcgaaaactctcaaggatcttaccgctgttgagatccagttcgatgtaacccactcgtgcaccc
aactgatcttcagcatcttttactttcaccagcgtttctgggtgagcaaaaacaggaaggcaaaatgccgcaaaaaagggaataag
ggcgacacggaaatgttgaatactcatactcttcctttttcaatattattgaagcatttatcagggttattgtctcatgagcggatacata
tttgaatgtatttagaaaaataaacaaataggggttccgcgcacatttccccgaaaagtgccacctgacgtctaagaaaccattatta
tcatgacattaacctataaaaataggcgtatcacgaggccctttcgtctcgcgcgtttcggtgatgacggtgaaaacctctgacacat
gcagctcccggagacggtcacagcttgtctgtaagcggatgccgggagcagacaagcccgtcagggcgcgtcagcgggtgttggc
gggtgtcggggctggcttaactatgcggcatcagagcagattgtactgagagtgcaccatatgcggtgtgaaataccgcacagatg
cgtaaggagaaaataccgcatcaggcgccattcgccattcaggctgcgcaactgttgggaagggcgatcggtgcgggcctcttcgc
tattacgccagctggcgaaagggggatgtgctgcaaggcgattaagttgggtaacgccagggttttcccagtcacgacgttgtaaa
acgacggccagtgaattc
pBRNGTR91 pTL20c MND WAS 650fwd (SEQ ID NO: 83)
ggccgcctcggccaaacagcccttgagtttaccactccctatcagtgatagagaaaagtgaaagtcgagtttaccactccctatcagt
gatagagaaaagtgaaagtcgagtttaccactccctatcagtgatagagaaaagtgaaagtcgagtttaccactccctatcagtgat
agagaaaagtgaaagtcgagtttaccagtccctatcagtgatagagaaaagtgaaagtcgagtttaccactccctatcagtgatag
agaaaagtgaaagtcgagtttaccactccctatcagtgatagagaaaagtgaaagtcgagctcgccatgggaggcgtggcctggg
cgggactggggagtggcgagccctcagatcctgcatataagcagctgctttttgcctgtactgggtctctctggttagaccagatctg
agcctgggagctctctggctaactagggaacccactgcttaagcctcaataaagcttgccttgagtgcttcaagtagtgtgtgcccgt
ctgttgtgtgactctggtaactagagatccctcagacccttttagtcagtgtggaaaatctctagcagtggcgcccgaacagggactt
gaaagcgaaagggaaaccagaggagctctctcgacgcaggactcggcttgctgaagcgcgcacggcaagaggcgaggggcgg
cgactggtgagtacgccaaaaattttgactagcggaggctagaaggagagagatgggtgcgagagcgtcagtattaagcggggg
agaattagatcgcgatgggaaaaaattcggttaaggccagggggaaagaaaaaatataaattaaaacatatagtatgggcaagc
agggagctagaacgattcgcagttaatactggcctgttagaaacatcagaaggctgtagacaaatactgggacagctacaaccatc
ccttcagacaggatcagaagaacttagatcattatataatacagtagcaaccctctattgtgtgcatcaaaggatagagataaaaga
caccaaggaagctttagacaagatagaggaagagcaaaacaaaagtaagaaaaaagcacagcaagcagcaggatcttcagac
ctggaaattccctacaatccccaaagtcaaggagtagtagaatctatgaataaagaattaaagaaaattataggacaggtaagag
atcaggctgaacatcttaagacagcagtacaaatggcagtattcatccacaattttaaaagaaaaggggggattggggggtacagt
gcaggggaaagaatagtagacataatagcaacagacatacaaactaaagaattacaaaaacaaattacaaaaattcaaaattttc
gggtttattacagggacagcagaaatccactttggaaaggaccagcaaagctcctctggaaaggtgaaggggcagtagtaataca
agataatagtgacataaaagtagtgccaagaagaaaagcaaagatcattagggattatggaaaacagatggcaggtgatgattg
tgtggcaagtagacaggatgaggattagaacatggaaaagtttagtaaaacaccataaggaggagatatgagggacaattggag
aagtgaattatataaatataaagtagtaaaaattgaaccattaggagtagcacccaccaaggcaaagagaagagtggtgcagag
agaaaaaagagcagtgggaataggagctttgttccttgggttcttgggagcagcaggaagcactatgggcgcagcgtcaatgacg
ctgacggtacaggccagacaattattgtctggtatagtgcagcagcagaacaatttgctgagggctattgaggcgcaacagcatct
gttgcaactcacagtctggggcatcaagcagctccaggcaagaatcctggctgtggaaagatacctaaaggatcaacagctcctgg
ggatttggggttgctctggaaaactcatttgcaccactgctgtgccttggaatgctagttggagtaataaatctctggaacagatttgg
aatcacacgacctggatggagtgggacagagaaattaacaattacacaagcttaatacactccttaattgaagaatcgcaaaacca
gcaagaaaagaatgaacaagaattattggaattagataaatgggcaagtttgtggaattggtttaacataacaaattggctgtggt
atataaaattattcataatgatagtaggaggcttggtaggtttaagaatagtttttgctgtactttctatagtgaatagagttaggcag
ggatattcaccattatcgtttcagacccacctcccaaccccgaggggaccgagctcaagcttcgaagcgatcgcacgcgtggatccg
aacagagagacagcagaatatgggccaaacaggatatctgtggtaagcagttcctgccccggctcagggccaagaacagttgga
acagcagaatatgggccaaacaggatatctgtggtaagcagttcctgccccggctcagggccaagaacagatggtccccagatgc
ggtcccgccctcagcagtttctagagaaccatcagatgtttccagggtgccccaaggacctgaaatgaccctgtgccttatttgaact
aaccaatcagttcgcttctcgcttctgttcgcgcgcttctgctccccgagctctatataagcagagctcgtttagtgaaccgtcagatcg
gcgcgccaattcaagcgagaagacaagggcagccgccaccatgagtgggggcccaatgggaggaaggcccgggggccgagga
gcaccagcggttcagcagaacataccctccaccctcctccaggaccacgagaaccagcgactctttgagatgcttggacgaaaatg
cttgacgctggccactgcagttgttcagctgtacctggcgctgccccctggagctgagcactggaccaaggagcattgtggggctgt
gtgcttcgtgaaggataacccccagaagtcctacttcatccgcctttacggccttcaggctggtcggctgctctgggaacaggagctg
tactcacagcttgtctactccacccccacccccttcttccacaccttcgctggagatgactgccaagcggggctgaactttgcagacga
ggacgaggcccaggccttccgggcactcgtgcaggagaagatacaaaaaaggaatcagaggcaaagtggagacagacgccag
ctacccccaccaccaacaccagccaatgaagagagaagaggagggctcccacccctgcccctgcatccaggtggagaccaagga
ggccctccagtgggtccgctctccctggggctggcgacagtggacatccagaaccctgacatcacgagttcacgataccgtgggctc
ccagcacctggacctagcccagctgataagaaacgctcagggaagaagaagatcagcaaagctgatattggtgcacccagtggat
tcaagcatgtcagccacgtggggtgggacccccagaatggatttgacgtgaacaacctcgacccagatctgcggagtctgttctcca
gggcaggaatcagcgaggcccagctcaccgacgccgagacctctaaacttatctacgacttcattgaggaccagggtgggctgga
ggctgtgcggcaggagatgaggcgccaggagccacttccgccgcccccaccgccatctcgaggagggaaccagctcccccggccc
cctattgtggggggtaacaagggtcgttctggtccactgccccctgtacctttggggattgccccacccccaccaacaccccggggac
ccccacccccaggccgagggggtcctccaccaccaccccctccagctactggacgttctggaccactgccccctccaccccctggag
ctggtgggccacccatgccaccaccaccgccaccaccgccaccgccgcccagctccgggaatggaccagcccctcccccactccctc
ctgctctggtgcctgccgggggcctggcccctggtgggggtcggggagcgcttttggatcaaatccggcagggaattcagctgaac
aagacccctggggccccagagagctcagcgctgcagccaccacctcagagctcagagggactggtgggggccctgatgcacgtg
atgcagaagagaagcagagccatccactcctccgacgaaggggaggaccaggctggcgatgaagatgaagatgatgaatggga
tgactgataactagtaatcaacctctggattacaaaatttgtgaaagattgactggtattcttaactatgttgctccttttacgctatgtg
gatacgctgctttaatgcctttgtatcatgctattgcttcccgtatggctttcattttctcctccttgtataaatcctggttgctgtctcttt
atgaggagttgtggcccgttgtcaggcaacgtggcgtggtgtgcactgtgtttgctgacgcaacccccactggttggggcattgccacc
acctgtcagctcctttccgggactttcgctttccccctccctattgccacggcggaactcatcgccgcctgccttgcccgctgctggaca
ggggctcggctgttgggcactgacaattccgtggtgttgtcggggaaatcatcgtcctttccttggctgctcgcctgtgttgccacctgg
attctgcgcgggacgtccttctgctacgtcccttcggccctcaatccagcggaccttccttcccgcggcctgctgccggctctgcggcct
cttccgcgtcttcgccttcgccctcagacgagtcggatctccctttgggccgcctccccgcacgtacgaccggtgcggccgcatcgatg
ccgtagtacctttaagaccaatgacttacaaggcagctgtagatcttagccactttttaaaagaaaaggggggactggaagggcta
attcactcccaaagaagacaagatggggagctcacggggacagcccccccccaaagcccccagggatgtaattacgtccctccccc
gctagggggcagcagcgagccgcccggggctccgctccggtccggcgctccccccgcatccccgagccggcagcgtgcggggac
agcccgggcacggggaaggtggcacgggatcgctttcctctgaacgcttctcgctgctctttgagcctgcagacacctggggggata
cggggaaaaagcttgatatcatgtgtctgagcctgcatgtttgatggtgtctggatgcaagcagaaggggtggaagagcttgcctg
gagagatacagctgggtcagtaggactgggacaggcagctggagaattgccatgtagatgttcatacaatcgtcaaatcatgaag
gctggaaaagccctccaagatccccaagaccaaccccaacccacccaccgtgcccactggccatgtccctcagtgccacatccccac
agttcttcatcacctccagggacggtgacccccccacctccgtgggcagctgtgccactgcagcaccgctctttggagaaggtaaatc
ttgctaaatccagcccgaccctcccctggcacaacgtaaggccattatctctcatccaactccaggacggagtcagtgaggatgggg
ctagatctgctttttgcctgtactgggtctctctggttagaccagatctgagcctgggagctctctggctaactagggaacccactgctt
aagcctcaataaagcttcagctgctcgagctagcagatctttttccctctgccaaaaattatggggacatcatgaagccccttgagcat
ctgacttctggctaataaaggaaatttattttcattgcaatagtgtgttggaattttttgtgtctctcactcggaaggacatatgggagg
gcaaatcatttaaaacatcagaatgagtatttggtttagagtttggcaacatatgcccatatgctggctgccatgaacaaaggttggc
tataaagaggtcatcagtatatgaaacagccccctgctgtccattccttattccatagaaaagccttgacttgaggttagattttttttat
attttgttttgtgttatttttttctttaacatccctaaaattttccttacatgttttactagccagatttttcctcctctcctgactactccc
agtcatagctgtccctcttctcttatggagatccctcgacctgcagcccaagcttggcgtaatcatggtcatagctgtttcctgtgtgaaatt
gttatccgctcacaattccacacaacatacgagccggaagcataaagtgtaaagcctggggtgcctaatgagtgagctaactcacatta
attgcgttgcgctcactgcccgctttccagtcgggaaacctgtcgtgccagcggatccgcatctcaattagtcagcaaccatagtcccg
cccctaactccgcccatcccgcccctaactccgcccagttccgcccattctccgccccatggctgactaattttttttatttatgcagagg
ccgaggccgcctcggcctctgagctattccagaagtagtgaggaggcttttttggaggcctaggcttttgcaaaaagctgtcgactgc
agaggcctgcatgcaagcttggcgtaatcatggtcatagctgtttcctgtgtgaaattgttatccgctcacaattccacacaacatacg
agccggaagcataaagtgtaaagcctggggtgcctaatgagtgagctaactcacattaattgcgttgcgctcactgcccgctttcca
gtcgggaaacctgtcgtgccagctgcattaatgaatcggccaacgcgcggggagaggcggtttgcgtattgggcgctcttccgcttc
ctcgctcactgactcgctgcgctcggtcgttcggctgcggcgagcggtatcagctcactcaaaggcggtaatacggttatccacaga
atcaggggataacgcaggaaagaacatgtgagcaaaaggccagcaaaaggccaggaaccgtaaaaaggccgcgttgctggcgt
ttttccataggctccgcccccctgacgagcatcacaaaaatcgacgctcaagtcagaggtggcgaaacccgacaggactataaaga
taccaggcgtttccccctggaagctccctcgtgcgctctcctgttccgaccctgccgcttaccggatacctgtccgcctttctcccttcgg
gaagcgtggcgctttctcatagctcacgctgtaggtatctcagttcggtgtaggtcgttcgctccaagctgggctgtgtgcacgaaccc
cccgttcagcccgaccgctgcgccttatccggtaactatcgtcttgagtccaacccggtaagacacgacttatcgccactggcagcag
ccactggtaacaggattagcagagcgaggtatgtaggcggtgctacagagttcttgaagtggtggcctaactacggctacactaga
agaacagtatttggtatctgcgctctgctgaagccagttaccttcggaaaaagagttggtagctcttgatccggcaaacaaaccacc
gctggtagcggtggtttttttgtttgcaagcagcagattacgcgcagaaaaaaaggatctcaagaagatcctttgatcttttctacggg
gtctgacgctcagtggaacgaaaactcacgttaagggattttggtcatgagattatcaaaaaggatcttcacctagatccttttaaatt
aaaaatgaagttttaaatcaatctaaagtatatatgagtaaacttggtctgacagttaccaatgcttaatcagtgaggcacctatctca
gcgatctgtctatttcgttcatccatagttgcctgactccccgtcgtgtagataactacgatacgggagggcttaccatctggccccagt
gctgcaatgataccgcgagacccacgctcaccggctccagatttatcagcaataaaccagccagccggaagggccgagcgcaga
agtggtcctgcaactttatccgcctccatccagtctattaattgttgccgggaagctagagtaagtagttcgccagttaatagtttgcgc
aacgttgttgccattgctacaggcatcgtggtgtcacgctcgtcgtttggtatggcttcattcagctccggttcccaacgatcaaggcg
agttacatgatcccccatgttgtgcaaaaaagcggttagctccttcggtcctccgatcgttgtcagaagtaagttggccgcagtgttat
cactcatggttatggcagcactgcataattctcttactgtcatgccatccgtaagatgcttttctgtgactggtgagtactcaaccaagtc
attctgagaatagtgtatgcggcgaccgagttgctcttgcccggcgtcaatacgggataataccgcgccacatagcagaactttaaa
agtgctcatcattggaaaacgttcttcggggcgaaaactctcaaggatcttaccgctgttgagatccagttcgatgtaacccactcgtg
cacccaactgatcttcagcatcttttactttcaccagcgtttctgggtgagcaaaaacaggaaggcaaaatgccgcaaaaaaggga
ataagggcgacacggaaatgttgaatactcatactcttcctttttcaatattattgaagcatttatcagggttattgtctcatgagcgga
tacatatttgaatgtatttagaaaaataaacaaataggggttccgcgcacatttccccgaaaagtgccacctgacgtctaagaaacc
attattatcatgacattaacctataaaaataggcgtatcacgaggccctttcgtctcgcgcgtttcggtgatgacggtgaaaacctctg
acacatgcagctcccggagacggtcacagcttgtctgtaagcggatgccgggagcagacaagcccgtcagggcgcgtcagcggg
tgttggcgggtgtcggggctggcttaactatgcggcatcagagcagattgtactgagagtgcaccatatgcggtgtgaaataccgca
cagatgcgtaaggagaaaataccgcatcaggcgccattcgccattcaggctgcgcaactgttgggaagggcgatcggtgcgggcc
tcttcgctattacgccagctggcgaaagggggatgtgctgcaaggcgattaagttgggtaacgccagggttttcccagtcacgacgt
tgtaaaacgacggccagtgaattc
pBRNGTR119 pTL20c MND WAS 650 3xSAmut (SEQ ID NO: 84)
ggccgcctcggccaaacagcccttgagtttaccactccctatcagtgatagagaaaagtgaaagtcgagtttaccactccctatcagt
gatagagaaaagtgaaagtcgagtttaccactccctatcagtgatagagaaaagtgaaagtcgagtttaccactccctatcagtgat
agagaaaagtgaaagtcgagtttaccagtccctatcagtgatagagaaaagtgaaagtcgagtttaccactccctatcagtgatag
agaaaagtgaaagtcgagtttaccactccctatcagtgatagagaaaagtgaaagtcgagctcgccatgggaggcgtggcctggg
cgggactggggagtggcgagccctcagatcctgcatataagcagctgctttttgcctgtactgggtctctctggttagaccagatctg
agcctgggagctctctggctaactagggaacccactgcttaagcctcaataaagcttgccttgagtgcttcaagtagtgtgtgcccgt
ctgttgtgtgactctggtaactagagatccctcagacccttttagtcagtgtggaaaatctctagcagtggcgcccgaacagggactt
gaaagcgaaagggaaaccagaggagctctctcgacgcaggactcggcttgctgaagcgcgcacggcaagaggcgaggggcgg
cgactggtgagtacgccaaaaattttgactagcggaggctagaaggagagagatgggtgcgagagcgtcagtattaagcggggg
agaattagatcgcgatgggaaaaaattcggttaaggccagggggaaagaaaaaatataaattaaaacatatagtatgggcaagc
agggagctagaacgattcgcagttaatactggcctgttagaaacatcagaaggctgtagacaaatactgggacagctacaaccatc
ccttcagacaggatcagaagaacttagatcattatataatacagtagcaaccctctattgtgtgcatcaaaggatagagataaaaga
caccaaggaagctttagacaagatagaggaagagcaaaacaaaagtaagaaaaaagcacagcaagcagcaggatcttcagac
ctggaaattccctacaatccccaaagtcaaggagtagtagaatctatgaataaagaattaaagaaaattataggacaggtaagag
atcaggctgaacatcttaagacagcagtacaaatggcagtattcatccacaattttaaaagaaaaggggggattggggggtacagt
gcaggggaaagaatagtagacataatagcaacagacatacaaactaaagaattacaaaaacaaattacaaaaattcaaaattttc
gggtttattacagggacagcagaaatccactttggaaaggaccagcaaagctcctctggaaaggtgaaggggcagtagtaataca
agataatagtgacataaaagtagtgccaagaagaaaagcaaagatcattagggattatggaaaacagatggcaggtgatgattg
tgtggcaagtagacaggatgaggattagaacatggaaaagtttagtaaaacaccataaggaggagatatgagggacaattggag
aagtgaattatataaatataaagtagtaaaaattgaaccattaggagtagcacccaccaaggcaaagagaagagtggtgcagag
agaaaaaagagcagtgggaataggagctttgttccttgggttcttgggagcagcaggaagcactatgggcgcagcgtcaatgacg
ctgacggtacaggccagacaattattgtctggtatagtgcagcagcagaacaatttgctgagggctattgaggcgcaacagcatct
gttgcaactcacagtctggggcatcaagcagctccaggcaagaatcctggctgtggaaagatacctaaaggatcaacagctcctgg
ggatttggggttgctctggaaaactcatttgcaccactgctgtgccttggaatgctagttggagtaataaatctctggaacagatttgg
aatcacacgacctggatggagtgggacagagaaattaacaattacacaagcttaatacactccttaattgaagaatcgcaaaacca
gcaagaaaagaatgaacaagaattattggaattagataaatgggcaagtttgtggaattggtttaacataacaaattggctgtggt
atataaaattattcataatgatagtaggaggcttggtaggtttaagaatagtttttgctgtactttctatagtgaatagagttaggcag
ggatattcaccattatcgtttcagacccacctcccaaccccgaggggaccgagctcaagcttcgaagcgatcgcacgcgtggatccg
aacagagagacagcagaatatgggccaaacaggatatctgtggtaagcagttcctgccccggctcagggccaagaacagttgga
acagcagaatatgggccaaacaggatatctgtggtaagcagttcctgccccggctcagggccaagaacagatggtccccagatgc
ggtcccgccctcagcagtttctagagaaccatcagatgtttccagggtgccccaaggacctgaaatgaccctgtgccttatttgaact
aaccaatcagttcgcttctcgcttctgttcgcgcgcttctgctccccgagctctatataagcagagctcgtttagtgaaccgtcagatcg
gcgcgccaattcaagcgagaagacaagggcagccgccaccatgagtgggggcccaatgggaggaaggcccgggggccgagga
gcaccagcggttcagcagaacataccctccaccctcctccaggaccacgagaaccagcgactctttgagatgcttggacgaaaatg
cttgacgctggccactgcagttgttcagctgtacctggcgctgccccctggagctgagcactggaccaaggagcattgtggggctgt
gtgcttcgtgaaggataacccccagaagtcctacttcatccgcctttacggccttcaggctggtcggctgctctgggaacaggagctg
tactcacagcttgtctactccacccccacccccttcttccacaccttcgctggagatgactgccaagcggggctgaactttgcagacga
ggacgaggcccaggccttccgggcactcgtgcaggagaagatacaaaaaaggaatcagaggcaaagtggagacagacgccag
ctacccccaccaccaacaccagccaatgaagagagaagaggagggctcccacccctgcccctgcatccaggtggagaccaagga
ggccctccagtgggtccgctctccctggggctggcgacagtggacatccagaaccctgacatcacgagttcacgataccgtgggctc
ccagcacctggacctagcccagctgataagaaacgctcagggaagaagaagatcagcaaagctgatattggtgcacccagtggat
tcaagcatgtcagccacgtggggtgggacccccagaatggatttgacgtgaacaacctcgacccagatctgcggagtctgttctcca
gggcaggaatcagcgaggcccagctcaccgacgccgagacctctaaacttatctacgacttcattgaggaccagggtgggctgga
ggctgtgcggcaggagatgaggcgccaggagccacttccgccgcccccaccgccatctcgaggagggaaccagctcccccggccc
cctattgtggggggtaacaagggtcgttctggtccactgccccctgtacctttggggattgccccacccccaccaacaccccggggac
ccccacccccaggccgagggggtcctccaccaccaccccctccagctactggacgttctggaccactgccccctccaccccctggag
ctggtgggccacccatgccaccaccaccgccaccaccgccaccgccgcccagctccgggaatggaccagcccctcccccactccctc
ctgctctggtgcctgccgggggcctggcccctggtgggggtcggggagcgcttttggatcaaatccggcagggaattcagctgaac
aagacccctggggccccagagagctcagcgctgcagccaccacctcagagctcagagggactggtgggggccctgatgcacgtg
atgcagaagagaagcagagccatccactcctccgacgaaggggaggaccaggctggcgatgaagatgaagatgatgaatggga
tgactgataactagtaatcaacctctggattacaaaatttgtgaaagattgactggtattcttaactatgttgctccttttacgctatgtg
gatacgctgctttaatgcctttgtatcatgctattgcttcccgtatggctttcattttctcctccttgtataaatcctggttgctgtctcttt
atgaggagttgtggcccgttgtcaggcaacgtggcgtggtgtgcactgtgtttgctgacgcaacccccactggttggggcattgccacc
acctgtcagctcctttccgggactttcgctttccccctccctattgccacggcggaactcatcgccgcctgccttgcccgctgctggaca
ggggctcggctgttgggcactgacaattccgtggtgttgtcggggaaatcatcgtcctttccttggctgctcgcctgtgttgccacctgg
attctgcgcgggacgtccttctgctacgtcccttcggccctcaatccagcggaccttccttcccgcggcctgctgccggctctgcggcct
cttccgcgtcttcgccttcgccctcagacgagtcggatctccctttgggccgcctccccgcacgtacgaccggtgcggccgcatcgatg
ccgtagtacctttaagaccaatgacttacaaggcagctgtagatcttagccactttttaaaagaaaaggggggactggaagggcta
attcactcccaaagaagacaagatagccccatcctcactgactccgtcctggagttggatgagagataatggccttacgttgtgcca
ggggagggtcgggctggatttagcaagatttaccttctccaaagagcggtgctgcagtggcacagctgcccacggaggtgggggg
gtcaccgtccctggaggtgatgaagaactgtggggatgtggcactgagggacatggccagtgggcacggtgggtgggttggggtt
ggtcttggggatcttggagggcttttccagccttcatgatttgacgattgtatgaacatctacatggcaattctccagctgcctgtccca
gtcctactgacccagctgtatctctccaggcaagctcttccaccccttctgcttgcatcctgacaccatcaaacatgcaggctcagacac
atgatatcaagctttttccccgtatcccccctggtgtctgctggctcaaagagcagcgagaagcgttcagaggaaagcgatcccgtg
ccaccttccccgtgcccgggctgtccccgcacgctgccggctcggggatgcggggggagcgccggaccggagcggagccccggg
cggctcgctgctgccccctagcgggggagggacgtaattacatccctgggggctttgggggggggctgtccccgtgagctccccag
atctgctttttgcctgtactgggtctctctggttagaccagatctgagcctgggagctctctggctaactagggaacccactgcttaagc
ctcaataaagcttcagctgctcgagctagcagatctttttccctctgccaaaaattatggggacatcatgaagccccttgagcatctga
cttctggctaataaaggaaatttattttcattgcaatagtgtgttggaattttttgtgtctctcactcggaaggacatatgggagggcaa
atcatttaaaacatcagaatgagtatttggtttagagtttggcaacatatgcccatatgctggctgccatgaacaaaggttggctataa
agaggtcatcagtatatgaaacagccccctgctgtccattccttattccatagaaaagccttgacttgaggttagattttttttatattttg
ttttgtgttatttttttctttaacatccctaaaattttccttacatgttttactagccagatttttcctcctctcctgactactcccagtcat
agctgtccctcttctcttatggagatccctcgacctgcagcccaagcttggcgtaatcatggtcatagctgtttcctgtgtgaaattgttatc
cgctcacaattccacacaacatacgagccggaagcataaagtgtaaagcctggggtgcctaatgagtgagctaactcacattaattgc
gttgcgctcactgcccgctttccagtcgggaaacctgtcgtgccagcggatccgcatctcaattagtcagcaaccatagtcccgcccct
aactccgcccatcccgcccctaactccgcccagttccgcccattctccgccccatggctgactaattttttttatttatgcagaggccgag
gccgcctcggcctctgagctattccagaagtagtgaggaggcttttttggaggcctaggcttttgcaaaaagctgtcgactgcagag
gcctgcatgcaagcttggcgtaatcatggtcatagctgtttcctgtgtgaaattgttatccgctcacaattccacacaacatacgagcc
ggaagcataaagtgtaaagcctggggtgcctaatgagtgagctaactcacattaattgcgttgcgctcactgcccgctttccagtcgg
gaaacctgtcgtgccagctgcattaatgaatcggccaacgcgcggggagaggcggtttgcgtattgggcgctcttccgcttcctcgct
cactgactcgctgcgctcggtcgttcggctgcggcgagcggtatcagctcactcaaaggcggtaatacggttatccacagaatcagg
ggataacgcaggaaagaacatgtgagcaaaaggccagcaaaaggccaggaaccgtaaaaaggccgcgttgctggcgtttttcca
taggctccgcccccctgacgagcatcacaaaaatcgacgctcaagtcagaggtggcgaaacccgacaggactataaagataccag
gcgtttccccctggaagctccctcgtgcgctctcctgttccgaccctgccgcttaccggatacctgtccgcctttctcccttcgggaagcg
tggcgctttctcatagctcacgctgtaggtatctcagttcggtgtaggtcgttcgctccaagctgggctgtgtgcacgaaccccccgttc
agcccgaccgctgcgccttatccggtaactatcgtcttgagtccaacccggtaagacacgacttatcgccactggcagcagccactg
gtaacaggattagcagagcgaggtatgtaggcggtgctacagagttcttgaagtggtggcctaactacggctacactagaagaac
agtatttggtatctgcgctctgctgaagccagttaccttcggaaaaagagttggtagctcttgatccggcaaacaaaccaccgctggt
agcggtggtttttttgtttgcaagcagcagattacgcgcagaaaaaaaggatctcaagaagatcctttgatcttttctacggggtctga
cgctcagtggaacgaaaactcacgttaagggattttggtcatgagattatcaaaaaggatcttcacctagatccttttaaattaaaaa
tgaagttttaaatcaatctaaagtatatatgagtaaacttggtctgacagttaccaatgcttaatcagtgaggcacctatctcagcgat
ctgtctatttcgttcatccatagttgcctgactccccgtcgtgtagataactacgatacgggagggcttaccatctggccccagtgctgc
aatgataccgcgagacccacgctcaccggctccagatttatcagcaataaaccagccagccggaagggccgagcgcagaagtggt
cctgcaactttatccgcctccatccagtctattaattgttgccgggaagctagagtaagtagttcgccagttaatagtttgcgcaacgtt
gttgccattgctacaggcatcgtggtgtcacgctcgtcgtttggtatggcttcattcagctccggttcccaacgatcaaggcgagttac
atgatcccccatgttgtgcaaaaaagcggttagctccttcggtcctccgatcgttgtcagaagtaagttggccgcagtgttatcactca
tggttatggcagcactgcataattctcttactgtcatgccatccgtaagatgcttttctgtgactggtgagtactcaaccaagtcattctg
agaatagtgtatgcggcgaccgagttgctcttgcccggcgtcaatacgggataataccgcgccacatagcagaactttaaaagtgct
catcattggaaaacgttcttcggggcgaaaactctcaaggatcttaccgctgttgagatccagttcgatgtaacccactcgtgcaccc
aactgatcttcagcatcttttactttcaccagcgtttctgggtgagcaaaaacaggaaggcaaaatgccgcaaaaaagggaataag
ggcgacacggaaatgttgaatactcatactcttcctttttcaatattattgaagcatttatcagggttattgtctcatgagcggatacata
tttgaatgtatttagaaaaataaacaaataggggttccgcgcacatttccccgaaaagtgccacctgacgtctaagaaaccattatta
tcatgacattaacctataaaaataggcgtatcacgaggccctttcgtctcgcgcgtttcggtgatgacggtgaaaacctctgacacat
gcagctcccggagacggtcacagcttgtctgtaagcggatgccgggagcagacaagcccgtcagggcgcgtcagcgggtgttggc
gggtgtcggggctggcttaactatgcggcatcagagcagattgtactgagagtgcaccatatgcggtgtgaaataccgcacagatg
cgtaaggagaaaataccgcatcaggcgccattcgccattcaggctgcgcaactgttgggaagggcgatcggtgcgggcctcttcgc
tattacgccagctggcgaaagggggatgtgctgcaaggcgattaagttgggtaacgccagggttttcccagtcacgacgttgtaaa
acgacggccagtgaattc
Unmodified γ-globin expression cassette (exons in uppercase) (SEQ ID NO: 85)
gtaaatacacttgcaaaggaggatgtttttagtagcaatttgtactgatggtatggggccaagagatatatcttagagggagggctg
agggtttgaagtccaactcctaagccagtgccagaagagccaaggacaggtacggctgtcatcacttagacctcaccctgtggagc
cacaccctagggttggccaatctactcccaggagcagggagggcaggagccagggctgggcataaaagtcagggcagagccatc
tattgcttacatttgcttctgacacaactgtgttcactagcaacctcaaacagacaccATGGGTCATTTCACAGAGGAGGAC
AAGGCTACTATCACAAGCCTGTGGGACAAGGTGAATGTGGAAGATGCTGGAGGAGAAACCCTGGGA
AGgtaggctctggtgaccaggacaagggagggaaggaaggaccctgtgcctggcaaaagtccaggttgcttctcaggatttgtgg
caccttctgactgtcaaactgttcttgtcaatctcacagGCTCCTGGTTGTCTACCCATGGACCCAGAGGTTCTTTGA
CAGCTTTGGCAACCTGTCCTCTGCCTCTGCCATCATGGGCAACCCCAAAGTCAAGGCACATGGCAAG
AAGGTGCTGACTTCCTTGGGAGATGCCATAAAGCACCTGGATGATCTCAAGGGCACCTTTGCCCAGC
TGAGTGAACTGCACTGTGACAAGCTGCATGTGGATCCTGAGAACTTCAAGgtgagtctatgggacccttgatg
ttttctttccccttcttttctatggttaagttcatgtcataggaaggggagaagtaacagggtacacatattgaccaaatcagggtaatt
ttgcatttgtaattttaaaaaatgctttcttcttttaatatacttttttgtttatcttatttctaatactttccctaatctctttctttcaggg
caataatgatacaatgtatcatgcctctttgcaccattctaaagaataacagtgataatttctgggttaaggcaatagcaatatttctgcatat
aaatatttctgcatataaattgtaactgatgtaagaggtttcatattgctaatagcagctacaatccagctaccattctgcttttattttat
ggttgggataaggctggattattctgagtccaagctaggcccttttgctaatcatgttcatacctcttatcttcctcccacagCTCCTG
GGCAACGTGCTGGTCACCGTGCTGGCCATTCACTTTGGCAAAGAATTCACCCCTGAGGTGCAGGCTT
CCTGGCAGAAGATGGTGACTGCAGTGGCCAGTGCCCTGTCCTCCAGATACCACTGAGcctcttgcccatga
ttcagagctttcaaggataggctttattctgcaagcaatacaaataataaatctattctgctgagagatcacacatgattttcttcagct
cttttttttacatctttttaaatatatgagccacaaagggtttatattgagggaagtgtgtatgtgtatttctgcatgcctgtttgtgtttgt
ggtgtgtgcatgctcctcatttatttttatatgagatgtgcattttgttgagcaaataaaagcagtaaagacacttgtacacgggagttc
tgcaagtgggagtaaatggtgtaggagaaatccggtgggaagaaagacctctataggacaggacttctcagaaacagatgttttg
gaagagatgggaaaaggttcagtgaagacctgggggctggattgattgcagctgagtagcaaggatggttcttaatgaagggaa
agtgttccaagctcggctagccggtgctagtctcccggaactatcactctttcacagtctgctttggaaggactgggcttagtatgaaa
agttaggactgagaagaatttgaaagggggctttttgtagcttgatattcactactgtcttattaccctatcataggcccaccccaaat
ggaagtcccattcttcctcaggatgtttaagattagcattcaggaagagatcagaggtctgctggctcccttatcatgtcccttatggtg
cttctggctccggctagcaccggtgatgatcctcgcgagctcgactctagaggatcccc
Unmodified γ-globin expression cassette-reverse complement (exons in uppercase) (SEQ
ID NO: 86)
ggggatcctctagagtcgagctcgcgaggatcatcaccggtgctagccggagccagaagcaccataagggacatgataagggag
ccagcagacctctgatctcttcctgaatgctaatcttaaacatcctgaggaagaatgggacttccatttggggtgggcctatgatagg
gtaataagacagtagtgaatatcaagctacaaaaagccccctttcaaattcttctcagtcctaacttttcatactaagcccagtccttcc
aaagcagactgtgaaagagtgatagttccgggagactagcaccggctagccgagcttggaacactttcccttcattaagaaccatc
cttgctactcagctgcaatcaatccagcccccaggtcttcactgaaccttttcccatctcttccaaaacatctgtttctgagaagtcctgtc
ctatagaggtctttcttcccaccggatttctcctacaccatttactcccacttgcagaactcccgtgtacaagtgtctttactgcttttattt
gctcaacaaaatgcacatctcatataaaaataaatgaggagcatgcacacaccacaaacacaaacaggcatgcagaaatacacat
acacacttccctcaatataaaccctttgtggctcatatatttaaaaagatgtaaaaaaaagagctgaagaaaatcatgtgtgatctctc
agcagaatagatttattatttgtattgcttgcagaataaagcctatccttgaaagctctgaatcatgggcaagaggCTCAGTGGT
ATCTGGAGGACAGGGCACTGGCCACTGCAGTCACCATCTTCTGCCAGGAAGCCTGCACCTCAGGGGT
GAATTCTTTGCCAAAGTGAATGGCCAGCACGGTGACCAGCACGTTGCCCAGGAGctgtgggaggaagata
agaggtatgaacatgattagcaaaagggcctagcttggactcagaataatccagccttatcccaaccataaaataaaagcagaatg
gtagctggattgtagctgctattagcaatatgaaacctcttacatcagttacaatttatatgcagaaatatttatatgcagaaatattgc
tattgccttaacccagaaattatcactgttattctttagaatggtgcaaagaggcatgatacattgtatcattattgccctgaaagaaag
agattagggaaagtattagaaataagataaacaaaaaagtatattaaaagaagaaagcattttttaaaattacaaatgcaaaatta
ccctgatttggtcaatatgtgtaccctgttacttctccccttcctatgacatgaacttaaccatagaaaagaaggggaaagaaaacatc
aagggtcccatagactcacCTTGAAGTTCTCAGGATCCACATGCAGCTTGTCACAGTGCAGTTCACTCAGCT
GGGCAAAGGTGCCCTTGAGATCATCCAGGTGCTTTATGGCATCTCCCAAGGAAGTCAGCACCTTCTT
GCCATGTGCCTTGACTTTGGGGTTGCCCATGATGGCAGAGGCAGAGGACAGGTTGCCAAAGCTGTCA
AAGAACCTCTGGGTCCATGGGTAGACAACCAGGAGCctgtgagattgacaagaacagtttgacagtcagaaggtg
ccacaaatcctgagaagcaacctggacttttgccaggcacagggtccttccttccctcccttgtcctggtcaccagagcctacCTTCC
CAGGGTTTCTCCTCCAGCATCTTCCACATTCACCTTGTCCCACAGGCTTGTGATAGTAGCCTTGTCCTC
CTCTGTGAAATGACCCATggtgtctgtttgaggttgctagtgaacacagttgtgtcagaagcaaatgtaagcaatagatggct
ctgccctgacttttatgcccagccctggctcctgccctccctgctcctgggagtagattggccaaccctagggtgtggctccacagggt
gaggtctaagtgatgacagccgtacctgtccttggctcttctggcactggcttaggagttggacttcaaaccctcagccctccctctaa
gatatatctcttggccccataccatcagtacaaattgctactaaaaacatcctcctttgcaagtgtatttac
Unmodified truncated HBB intron 3 (SEQ ID NO: 87)
Gtgagtctatgggacccttgatgttttctttccccttcttttctatggttaagttcatgtcataggaaggggagaagtaacagggtacac
atattgaccaaatcagggtaattttgcatttgtaattttaaaaaatgctttcttcttttaatatacttttttgtttatcttatttctaatactt
tccctaatctctttctttcagggcaataatgatacaatgtatcatgcctctttgcaccattctaaagaataacagtgataatttctgggttaa
ggcaatagcaatatttctgcatataaatatttctgcatataaattgtaactgatgtaagaggtttcatattgctaatagcagctacaatc
cagctaccattctgcttttattttatggttgggataaggctggattattctgagtccaagctaggcccttttgctaatcatgttcatacctct
tatcttcctcccacag
Unmodified truncated HBB intron 3-reverse complement (SEQ ID NO: 88)
Ctgtgggaggaagataagaggtatgaacatgattagcaaaagggcctagcttggactcagaataatccagccttatcccaaccata
aaataaaagcagaatggtagctggattgtagctgctattagcaatatgaaacctcttacatcagttacaatttatatgcagaaatattt
atatgcagaaatattgctattgccttaacccagaaattatcactgttattctttagaatggtgcaaagaggcatgatacattgtatcatt
attgccctgaaagaaagagattagggaaagtattagaaataagataaacaaaaaagtatattaaaagaagaaagcattttttaaa
attacaaatgcaaaattaccctgatttggtcaatatgtgtaccctgttacttctccccttcctatgacatgaacttaaccatagaaaaga
aggggaaagaaaacatcaagggtcccatagactcac
Unmodified HS4-400 (SEQ ID NO: 89)
Ggggagctcacggggacagcccccccccaaagcccccagggatgtaattacgtccctcccccgctagggggcagcagcgagccg
cccggggctccgctccggtccggcgctccccccgcatccccgagccggcagcgtgcggggacagcccgggcacggggaaggtgg
cacgggatcgctttcctctgaacgcttctcgctgctctttgagcctgcagacacctggggggatacggggaaaaagctttaggctga
aagagagatttagaatgacagaatcatagaacggcctgggttgcaaaggagcacagtgctcatccagatccaaccccctgctatgt
gcagggtcatcaaccagcagcccaggctgcccagagccacatccagcctggccttgaatgcctgcagggat
Unmodified HS4-400-reverse complement (SEQ ID NO: 90)
Atccctgcaggcattcaaggccaggctggatgtggctctgggcagcctgggctgctggttgatgaccctgcacatagcagggggtt
ggatctggatgagcactgtgctcctttgcaacccaggccgttctatgattctgtcattctaaatctctctttcagcctaaagctttttcccc
gtatccccccaggtgtctgcaggctcaaagagcagcgagaagcgttcagaggaaagcgatcccgtgccaccttccccgtgcccggg
ctgtccccgcacgctgccggctcggggatgcggggggagcgccggaccggagcggagccccgggggctcgctgctgcccccta
gcgggggagggacgtaattacatccctgggggctttgggggggggctgtccccgtgagctcccc
Modified γ-globin transgene-reverse complement (mutation in bold and underlined) (SEQ
ID NO: 91)
ggggatcctctagagtcgagctcgcgaggatcatcaccggtgctagccggagccagaagcaccataagggacatgataagggag
ccagcagacctctgatctcttcctgaatgctaatcttaaacatcctgaggaagaatgggacttccatttggggtgggcctatgatagg
gtaataagacagtagtgaatatcaagctacaaaaagccccctttcaaattcttctcagtcctaacttttcatactaagcccagtccttcc
aaagcagactgtgaaagagtgatagttccgggagactagcaccggctagccgagcttggaacactttcccttcattaagaaccatc
cttgctactcagctgcaatcaatccagcccccaggtcttcactgaaccttttcccatctcttccaaaacatctgtttctgagaagtcctgtc
ctatagaggtctttcttcccaccggatttctcctacaccatttactcccacttgcagaactcccgtgtacaagtgtctttactgcttttattt
gctcaacaaaatgcacatctcatataaaaataaatgaggagcatgcacacaccacaaacacaaacaggcatgcagaaatacacat
acacacttccctcaatataaaccctttgtggctcatatatttaaaaagatgtaaaaaaaagagctgaagaaaatcatgtgtgatctctc
agcagaatagatttattatttgtattgcttgcagaataaagcctatccttgaaagctctgaatcatgggcaagaggCTCAGTGGT
ATCTGGAGGACAGGGCACTGGCCACTGCAGTCACCATCTTCTGCCAGGAAGCCTGCACCTCAGGGGT
GAATTCTTTGCCAAAGTGAATGGCCAGCACGGTGACCAGCACGTTGCCCAGGAGctgtgggaggaagata
agagatatgaacatgattagcaaaagggcctagcttggactcagaataatccagccttatcccaaccataaaataaaagcagaatg
gtagctggattgtagctgctattagcaatatgaaacctcttacatcagttacaatttatatgcagaaatatttatatgcagaaatattgc
tattgccttaacccagaaattatcactgttattctttagaatggtgcaaagaggcatgatacattgtatcattattgccctgaaagaaag
agattagggaaagtattagaaataagataaacaaaaaagtatattaaaagaagaaagcattttttaaaattacaaatgcaaaatta
ccctgatttggtcaatatgtgtaccctgttacttctccccttcctatgacatgaacttaaccatagaaaagaaggggaaagaaaacatc
aagggtcccatagactcacCTTGAAGTTCTCAGGATCCACATGCAGCTTGTCACAGTGCAGTTCACTCAGCT
GGGCAAAGGTGCCCTTGAGATCATCCAGGTGCTTTATGGCATCTCCCAAGGAAGTCAGCACCTTCTT
GCCATGTGCCTTGACTTTGGGGTTGCCCATGATGGCAGAGGCAGAGGACAGGTTGCCAAAGCTGTCA
AAGAACCTCTGGGTCCATGGGTAGACAACCAGGAGCctgtgagattgacaagaacagtttgacagtcagaaggtg
ccacaaatcctgagaagcaacctggacttttgccaggcacagggtccttccttccctcccttgtcctggtcaccagagcctacCTTCC
CAGGGTTTCTCCTCCAGCATCTTCCACATTCACCTTGTCCCACAGGCTTGTGATAGTAGCCTTGTCCTC
CTCTGTGAAATGACCCATggtgtctgtttgaggttgctagtgaacacagttgtgtcagaagcaaatgtaagcaatagatggct
ctgccctgacttttatgcccagccctggctcctgccctccctgctcctgggagtagattggccaaccctagggtgtggctccacagggt
gaggtctaagtgatgacagccgtacctgtccttggctcttctggcactggcttaggagttggacttcaaaccctcagccctccctctaa
gatatatctcttggccccataccatcagtacaaattgctactaaaaacatcctcctttgcaagtgtatttac
Modified truncated HBB intron 3-reverse complement (mutation in bold and underlined)
(SEQ ID NO: 92)
ctgtgggaggaagataagagatatgaacatgattagcaaaagggcctagcttggactcagaataatccagccttatcccaaccata
aaataaaagcagaatggtagctggattgtagctgctattagcaatatgaaacctcttacatcagttacaatttatatgcagaaatattt
atatgcagaaatattgctattgccttaacccagaaattatcactgttattctttagaatggtgcaaagaggcatgatacattgtatcatt
attgccctgaaagaaagagattagggaaagtattagaaataagataaacaaaaaagtatattaaaagaagaaagcattttttaaa
attacaaatgcaaaattaccctgatttggtcaatatgtgtaccctgttacttctccccttcctatgacatgaacttaaccatagaaaaga
aggggaaagaaaacatcaagggtcccatagactcac
Modified HS4-400-reverse complement-A to T mutation at SA2 (mutation in bold and
underlined) (SEQ ID NO: 93)
atccctgcaggcattcaaggccaggctggatgtggctctgggcagcctgggctgctggttgatgaccctgcacatagcagggggtt
ggatctggatgagcactgtgctcctttgcaacccaggccgttctatgattctgtcattctaaatctctctttcagcctaaagctttttcccc
gtatcccccctggtgtctgcaggctcaaagagcagcgagaagcgttcagaggaaagcgatcccgtgccaccttccccgtgcccggg
ctgtccccgcacgctgccggctcggggatgcggggggagcgccggaccggagcggagccccgggcggctcgctgctgcccccta
gcgggggagggacgtaattacatccctgggggctttgggggggggctgtccccgtgagctcccc
Modified HS4-400-reverse complement-A to T mutation at SA2 and SA3 (mutation in bold
and underlined) (SEQ ID NO: 94)
atccctgcaggcattcaaggccaggctggatgtggctctgggcagcctgggctgctggttgatgaccctgcacatagcagggggtt
ggatctggatgagcactgtgctcctttgcaacccaggccgttctatgattctgtcattctaaatctctctttcagcctaaagctttttcccc
gtatcccccctggtgtctgctggctcaaagagcagcgagaagcgttcagaggaaagcgatcccgtgccaccttccccgtgcccggg
ctgtccccgcacgctgccggctcggggatgcggggggagcgccggaccggagcggagccccgggggctcgctgctgcccccta
gcgggggagggacgtaattacatccctgggggctttgggggggggctgtccccgtgagctcccc
Modified HS4-400-reverse complement-A to T mutation at SA3 (mutation in bold and
underlined) (SEQ ID NO: 95)
atccctgcaggcattcaaggccaggctggatgtggctctgggcagcctgggctgctggttgatgaccctgcacatagcagggggtt
ggatctggatgagcactgtgctcctttgcaacccaggccgttctatgattctgtcattctaaatctctctttcagcctaaagctttttcccc
gtatccccccaggtgtctgctggctcaaagagcagcgagaagcgttcagaggaaagcgatcccgtgccaccttccccgtgcccggg
ctgtccccgcacgctgccggctcggggatgcggggggagcgccggaccggagcggagccccgggcggctcgctgctgcccccta
gcgggggagggacgtaattacatccctgggggctttgggggggggctgtccccgtgagctcccc
Splice donor site 1 (SD1) (SEQ ID NO: 96)
AAGATAAGAGGTATGAACAT
Inactivated SD1 (mutation underlined) (SEQ ID NO: 97)
AAGATAAGAGATATGAACAT
γ-globin Exon 1 (SEQ ID NO: 98)
CCTTCCCAGGGTTTCTCCTCCAGCATCTTCCACATTCACCTTGTCCCACAGGCTTGTGATAGTAGCCTT
GTCCTCCTCTGTGAAATGACCCAT
γ-globin Exon 2 (SEQ ID NO: 99)
CTTGAAGTTCTCAGGATCCACATGCAGCTTGTCACAGTGCAGTTCACTCAGCTGGGCAAAGGTGCCCT
TGAGATCATCCAGGTGCTTTATGGCATCTCCCAAGGAAGTCAGCACCTTCTTGCCATGTGCCTTGACT
TTGGGGTTGCCCATGATGGCAGAGGCAGAGGACAGGTTGCCAAAGCTGTCAAAGAACCTCTGGGTC
CATGGGTAGACAACCAGGAGC
γ-globin Exon 3 (SEQ ID NO: 100)
GTGATCTCTCAGCAGAATAGATTTATTATTTGTATTGCTTGCAGAATAAAGCCTATCCTTGAAAGCTCT
GAATCATGGGCAAGAGGCTCAGTGGTATCTGGAGGACAGGGCACTGGCCACTGCAGTCACCATCTTC
TGCCAGGAAGCCTGCACCTCAGGGGTGAATTCTTTGCCAAAGTGAATGGCCAGCACGGTGACCAGCA
CGTTGCCCAGGAG
γ-globin coding sequence (SEQ ID NO: 101)
CAGACACCATGGGTCATTTCACAGAGGAGGACAAGGCTACTATCACAAGCCTGTGGGACAAGGTGAA
TGTGGAAGATGCTGGAGGAGAAACCCTGGGAAGGCTCCTGGTTGTCTACCCATGGACCCAGAGGTTC
TTTGACAGCTTTGGCAACCTGTCCTCTGCCTCTGCCATCATGGGCAACCCCAAAGTCAAGGCACATGG
CAAGAAGGTGCTGACTTCCTTGGGAGATGCCATAAAGCACCTGGATGATCTCAAGGGCACCTTTGCC
CAGCTGAGTGAACTGCACTGTGACAAGCTGCATGTGGATCCTGAGAACTTCAAGCTCCTGGGCAACG
TGCTGGTCACCGTGCTGGCCATTCACTTTGGCAAAGAATTCACCCCTGAGGTGCAGGCTTCCTGGCA
GAAGATGGTGACTGCAGTGGCCAGTGCCCTGTCCTCCAGATACCACTGAGCCTCTTGCCCATGATTC
AGAGCTTTCAAGGATAGGCTTTATTCTGCAAGCAATACAAATAATAAATCTATTCTGCTGAGAGATCAC
γ-globin coding sequence (SEQ ID NO: 102)
ATGGGTCATTTCACAGAGGAGGACAAGGCTACTATCACAAGCCTGTGGGACAAGGTGAATGTGGAAG
ATGCTGGAGGAGAAACCCTGGGAAGGCTCCTGGTTGTCTACCCATGGACCCAGAGGTTCTTTGACAG
CTTTGGCAACCTGTCCTCTGCCTCTGCCATCATGGGCAACCCCAAAGTCAAGGCACATGGCAAGAAG
GTGCTGACTTCCTTGGGAGATGCCATAAAGCACCTGGATGATCTCAAGGGCACCTTTGCCCAGCTGA
GTGAACTGCACTGTGACAAGCTGCATGTGGATCCTGAGAACTTCAAGCTCCTGGGCAACGTGCTGGT
CACCGTGCTGGCCATTCACTTTGGCAAAGAATTCACCCCTGAGGTGCAGGCTTCCTGGCAGAAGATG
GTGACTGCAGTGGCCAGTGCCCTGTCCTCCAGATACCACTGA
γ-globin G16D (GbGMG16D) protein (SEQ ID NO: 103)
MGHFTEEDKATITSLWDKVNVEDAGGETLGRLLVVYPWTQRFFDSFGNLSSASAIMGNPKVKAHGKKVL
TSLGDAIKHLDDLKGTFAQLSELHCDKLHVDPENFKLLGNVLVTVLAIHFGKEFTPEVQASWQKMVTAVA
SALSSRYH
β-globin poly(A) signal (SEQ ID NO: 104)
GATCTTTTTCCCTCTGCCAAAAATTATGGGGACATCATGAAGCCCCTTGAGCATCTGACTTCTGGCTAA
TAAAGGAAATTTATTTTCATTGCAATAGTGTGTTGGAATTTTTTGTGTCTCTCACTCGGAAGGACATAT
GGGAGGGCAAATCATTTAAAACATCAGAATGAGTATTTGGTTTAGAGTTTGGCAACATATGCCCATAT
GCTGGCTGCCATGAACAAAGGTTGGCTATAAAGAGGTCATCAGTATATGAAACAGCCCCCTGCTGTC
CATTCCTTATTCCATAGAAAAGCCTTGACTTGAGGTTAGATTTTTTTTATATTTTGTTTTGTGTTATTTTT
TTCTTTAACATCCCTAAAATTTTCCTTACATGTTTTACTAGCCAGATTTTTCCTCCTCTCCTGACTACTCC
CAGTCATAGCTGTCCCTCTTCTCTTATGGAGATC
β-globin Locus control region (LCR) (SEQ ID NO: 105)
GTATGTGAGCATGTGTCCTCTAACAGCACAGGCCTTTTGCCACCTAGCTGTCCAGGGGTGCCTTAAAA
TGGCAAACAAGGTTTGTTTTCTTTTCCTGTTTTCATGCCTTCCTCTTCCATATCCTTGTTTCATATTAATA
CATGTGTATAGATCCTAAAAATCTATACACATGTATTAATAAAGCCTGATTCTGCCGCTTCTAGGTATA
GAGGCCACCTGCAAGATAAATATTTGATTCACAATAACTAATCATTCTATGGCAATTGATAACAACAAA
TATATATATATATATATATATACGTATATGTGTATATATATATATATATTCAGGAAATAATATATTCTAGA
ATATGTCACATTCTGTCTCAGGCATCCATTTTCTTTATGATGCCGTTTGAGGTGGAGTTTTAGTCAGGT
GGTCAGCTTCTCCTTTTTTTTGCCATCTGCCCTGTAAGCATCCTGCTGGGGACCCAGATAGGAGTCAT
CACTCTAGGCTGAGAACATCTGGGCACACACCCTAAGCCTCAGCATGACTCATCATGACTCAGCATTG
CTGTGCTTGAGCCAGAAGGTTTGCTTAGAAGGTTACACAGAACCAGAAGGCGGGGGTGGGGCACTG
ACCCCGACAGGGGCCTGGCCAGAACTGCTCATGCTTGGACTATGGGAGGTCACTAATGGAGACACAC
AGAAATGTAACAGGAACTAAGGGAATTCCGGTGCCCTGCTTAGGAGCTTAATCTTTAATGAAAGCTAA
GCTTTCATTAAAAAAAGTCTAACCAGCTGCATTCGACTTTGACTGCAGCAGCTGGTTAGAAGGTTCTA
CTGGAGGAGGGTCCCAGCCCATTGCTAAATTAACATCAGGCTCTGAGACTGGCAGTATATCTCTAACA
GTGGTTGATGCTATCTTCTGGAACTTGCCTGCTACATTGAGACCACTGACCCATACATAGGAAGCCCA
TAGCTCTGTCCTGAACTGTTAGGCCACTGGTCCAGAGAGTGTGCATCTCCTTTGATCCTCATAATAAC
CCTATGAGATAGACACAATTATTACTCTTACTTTATAGATGATGATCCTGAAAACATAGGAGTCAAGGC
ACTTGCCCCTAGCTGGGGGTATAGGGGAGCAGTCCCATGTAGTAGTAGAATGAAAAATGCTGCTATG
CTGTGCCTCCCCCACCTTTCCCATGTCTGCCCTCTACTCATGGTCTATCTCTCCTGGCTCCTGGGAGTC
ATGGACTCCACCCAGCACCACCAACCTGACCTAACCACCTATCTGAGCCTGCCAGCCTATAACCCATC
TGGGCCCTGATAGCTGGTGGCCAGCCCTGACCCCACCCCACCCTCCCTGGAACCTCTGATAGACACA
TCTGGCACACCAGCTCGCAAAGTCACCGTGAGGGTCTTGTGTTTGCTGAGTCAAAATTCCTTGAAATC
CAAGTCCTTAGAGACTCCTGCTCCCAAATTTACAGTCATAGACTTCTTCATGGCTGTCTCCTTTATCCA
CAGAATGATTCCTTTGCTTCATTGCCCCATCCATCTGATCCTCCTCATCAGTGCAGCACAGGGCCCAT
GAGCAGTAGCTGCAGAGTCTCACATAGGTCTGGCACTGCCTCTGACATGTCCGACCTTAGGCAAATG
CTTGACTCTTCTGAGCTCGGATCCCTTGAGCTCAGGAGGTCAAGGCTGCAGTGAGACATGATCTTGC
CACTGCACTCCAGCCTGGACAGCAGAGTGAAACCTTGCCTCACGAAACAGAATACAAAAACAAACAA
ACAAAAAACTGCTCCGCAATGCGCTTCCTTGATGCTCTACCACATAGGTCTGGGTACTTTGTACACATT
ATCTCATTGCTGTTCATAATTGTTAGATTAATTTTGTAATATTGATATTATTCCTAGAAAGCTGAGGCCT
CAAGATGATAACTTTTATTTTCTGGACTTGTAATAGCTTTCTCTTGTATTCACCATGTTGTAACTTTCTT
AGAGTAGTAACAATATAAAGTTATTGTGAGTTTTTGCAAACACAGCAAACACAACGACCCATATAGAC
ATTGATGTGAAATTGTCTATTGTCAATTTATGGGAAAACAAGTATGTACTTTTTCTACTAAGCCATTGA
AACAGGAATAACAGAACAAGATTGAAAGAATACATTTTCCGAAATTACTTGAGTATTATACAAAGACAA
GCACGTGGACCTGGGAGGAGGGTTATTGTCCATGACTGGTGTGTGGAGACAAATGCAGGTTTATAAT
AGATGGGATGGCATCTAGCGCAATGACTTTGCCATCACTTTTAGAGAGCTCTTGGGGGCCCCAGTAC
ACAAGAGGGGACGCAGGGTATATGTAGACATCTCATTCTTTTTCTTAGTGTGAGAATAAGAATAGCCA
TGACCTGAGTTTATAGACAATGAGCCCTTTTCTCTCTCCCACTCAGCAGCTATGAGATGGCTTGCCCT
GCCTCTCTACTAGGCTGACTCACTCCAAGGCCCAGCAATGGGCAGGGCTCTGTCAGGGCTTTGATAG
CACTATCTGCAGAGCCAGGGCCGAGAAGGGGTGGACTCCAGAGACTCTCCCTCCCATTCCCGAGCAG
GGTTTGCTTATTTATGCATTTAAATGATATATTTATTTTAAAAGAAATAACAGGAGACTGCCCAGCCCT
GGCTGTGACATGGAAACTATGTAGAATATTTTGGGTTCCATTTTTTTTTCCTTCTTTCAGTTAGAGGAA
AAGGGGCTCACTGCACATACACTAGACAGAAAGTCAGGAGCTTTGAATCCAAGCCTGATCATTTCCAT
GTCATACTGAGAAAGTCCCCACCCTTCTCTGAGCCTCAGTTTCTCTTTTTATAAGTAGGAGTCTGGAGT
AAATGATTTCCAATGGCTCTCATTTCAATACAAAATTTCCGTTTATTAAATGCATGAGCTTCCGTTACTC
CAAGACTGAGAAGGAAATTGAACCTGAGACTCATTGACTGGCAAGATGTCCCCAGAGGCTCTCATTCA
GCAATAAAATTCTCACCTTCACCCAGGCCCACTAGTGTCAGATTTGCATGC
REV response element (RRE) (SEQ ID NO: 106)
AGGAGGAGATATGAGGGACAATTGGAGAAGTGAATTATATAAATATAAAGTAGTAAAAATTGAACCAT
TAGGAGTAGCACCCACCAAGGCAAAGAGAAGAGTGGTGCAGAGAGAAAAAAGAGCAGTGGGAATAG
GAGCTTTGTTCCTTGGGTTCTTGGGAGCAGCAGGAAGCACTATGGGCGCAGCGTCAATGACGCTGAC
GGTACAGGCCAGACAATTATTGTCTGGTATAGTGCAGCAGCAGAACAATTTGCTGAGGGCTATTGAG
GCGCAACAGCATCTGTTGCAACTCACAGTCTGGGGCATCAAGCAGCTCCAGGCAAGAATCCTGGCTG
TGGAAAGATACCTAAAGGATCAACAGCTCCTGGGGATTTGGGGTTGCTCTGGAAAACTCATTTGCACC
ACTGCTGTGCCTTGGAATGCTAGTTGGAGTAATAAATCTCTGGAACAGATTTGGAATCACACGACCTG
GATGGAGTGGGACAGAGAAATTAACAATTACACAAGCTTAATACACTCCTTAATTGAAGAATCGCAAA
ACCAGCAAGAAAAGAATGAACAAGAATTATTGGAATTAGATAAATGGGCAAGTTTGTGGAATTGGTTT
AACATAACAAATTGGCTGTGGTATATAAAATTATTCATAATGATAGTAGGAGGCTTGGTAGGTTTAAGA
ATAGTTTTTGCTGTACTTTCTATAGTGAATAGAGTTAGGCAGGGATATTCACCATTATCGTTTCAGACC
CACCTCCCAACCCCGAGGGGACCG
pCalH10 TL20c rGbGM 7SKsh734 (SEQ ID NO: 107)
GGCCGCCTCGGCCAAACAGCCCTTGAGTTTACCACTCCCTATCAGTGATAGAGAAAAGTGAAAGTCG
AGTTTACCACTCCCTATCAGTGATAGAGAAAAGTGAAAGTCGAGTTTACCACTCCCTATCAGTGATAG
AGAAAAGTGAAAGTCGAGTTTACCACTCCCTATCAGTGATAGAGAAAAGTGAAAGTCGAGTTTACCAG
TCCCTATCAGTGATAGAGAAAAGTGAAAGTCGAGTTTACCACTCCCTATCAGTGATAGAGAAAAGTGA
AAGTCGAGTTTACCACTCCCTATCAGTGATAGAGAAAAGTGAAAGTCGAGCTCGCCATGGGAGGCGT
GGCCTGGGCGGGACTGGGGAGTGGCGAGCCCTCAGATCCTGCATATAAGCAGCTGCTTTTTGCCTGT
ACTGGGTCTCTCTGGTTAGACCAGATCTGAGCCTGGGAGCTCTCTGGCTAACTAGGGAACCCACTGC
TTAAGCCTCAATAAAGCTTGCCTTGAGTGCTTCAAGTAGTGTGTGCCCGTCTGTTGTGTGACTCTGGT
AACTAGAGATCCCTCAGACCCTTTTAGTCAGTGTGGAAAATCTCTAGCAGTGGCGCCCGAACAGGGA
CTTGAAAGCGAAAGGGAAACCAGAGGAGCTCTCTCGACGCAGGACTCGGCTTGCTGAAGCGCGCAC
GGCAAGAGGCGAGGGGCGGCGACTGGTGAGTACGCCAAAAATTTTGACTAGCGGAGGCTAGAAGGA
GAGAGATGGGTGCGAGAGCGTCAGTATTAAGCGGGGGAGAATTAGATCGCGATGGGAAAAAATTCG
GTTAAGGCCAGGGGGAAAGAAAAAATATAAATTAAAACATATAGTATGGGCAAGCAGGGAGCTAGAA
CGATTCGCAGTTAATACTGGCCTGTTAGAAACATCAGAAGGCTGTAGACAAATACTGGGACAGCTACA
ACCATCCCTTCAGACAGGATCAGAAGAACTTAGATCATTATATAATACAGTAGCAACCCTCTATTGTGT
GCATCAAAGGATAGAGATAAAAGACACCAAGGAAGCTTTAGACAAGATAGAGGAAGAGCAAAACAAA
AGTAAGAAAAAAGCACAGCAAGCAGCAGGATCTTCAGACCTGGAAATTCCCTACAATCCCCAAAGTCA
AGGAGTAGTAGAATCTATGAATAAAGAATTAAAGAAAATTATAGGACAGGTAAGAGATCAGGCTGAAC
ATCTTAAGACAGCAGTACAAATGGCAGTATTCATCCACAATTTTAAAAGAAAAGGGGGGATTGGGGG
GTACAGTGCAGGGGAAAGAATAGTAGACATAATAGCAACAGACATACAAACTAAAGAATTACAAAAAC
AAATTACAAAAATTCAAAATTTTCGGGTTTATTACAGGGACAGCAGAAATCCACTTTGGAAAGGACCA
GCAAAGCTCCTCTGGAAAGGTGAAGGGGCAGTAGTAATACAAGATAATAGTGACATAAAAGTAGTGC
CAAGAAGAAAAGCAAAGATCATTAGGGATTATGGAAAACAGATGGCAGGTGATGATTGTGTGGCAAG
TAGACAGGATGAGGATTAGAACATGGAAAAGTTTAGTAAAACACCATAAGGAGGAGATATGAGGGAC
AATTGGAGAAGTGAATTATATAAATATAAAGTAGTAAAAATTGAACCATTAGGAGTAGCACCCACCAA
GGCAAAGAGAAGAGTGGTGCAGAGAGAAAAAAGAGCAGTGGGAATAGGAGCTTTGTTCCTTGGGTT
CTTGGGAGCAGCAGGAAGCACTATGGGCGCAGCGTCAATGACGCTGACGGTACAGGCCAGACAATT
ATTGTCTGGTATAGTGCAGCAGCAGAACAATTTGCTGAGGGCTATTGAGGCGCAACAGCATCTGTTG
CAACTCACAGTCTGGGGCATCAAGCAGCTCCAGGCAAGAATCCTGGCTGTGGAAAGATACCTAAAGG
ATCAACAGCTCCTGGGGATTTGGGGTTGCTCTGGAAAACTCATTTGCACCACTGCTGTGCCTTGGAAT
GCTAGTTGGAGTAATAAATCTCTGGAACAGATTTGGAATCACACGACCTGGATGGAGTGGGACAGAG
AAATTAACAATTACACAAGCTTAATACACTCCTTAATTGAAGAATCGCAAAACCAGCAAGAAAAGAATG
AACAAGAATTATTGGAATTAGATAAATGGGCAAGTTTGTGGAATTGGTTTAACATAACAAATTGGCTGT
GGTATATAAAATTATTCATAATGATAGTAGGAGGCTTGGTAGGTTTAAGAATAGTTTTTGCTGTACTTT
CTATAGTGAATAGAGTTAGGCAGGGATATTCACCATTATCGTTTCAGACCCACCTCCCAACCCCGAGG
GGACCGAGCTCAAGCTTCGAACGCGTGGGGATCCTCTAGAGTCGAGCTCGCGAGGATCATCACCGG
TGCTAGCCGGAGCCAGAAGCACCATAAGGGACATGATAAGGGAGCCAGCAGACCTCTGATCTCTTCC
TGAATGCTAATCTTAAACATCCTGAGGAAGAATGGGACTTCCATTTGGGGTGGGCCTATGATAGGGTA
ATAAGACAGTAGTGAATATCAAGCTACAAAAAGCCCCCTTTCAAATTCTTCTCAGTCCTAACTTTTCAT
ACTAAGCCCAGTCCTTCCAAAGCAGACTGTGAAAGAGTGATAGTTCCGGGAGACTAGCACCGGCTAG
CCGAGCTTGGAACACTTTCCCTTCATTAAGAACCATCCTTGCTACTCAGCTGCAATCAATCCAGCCCCC
AGGTCTTCACTGAACCTTTTCCCATCTCTTCCAAAACATCTGTTTCTGAGAAGTCCTGTCCTATAGAGG
TCTTTCTTCCCACCGGATTTCTCCTACACCATTTACTCCCACTTGCAGAACTCCCGTGTACAAGTGTCT
TTACTGCTTTTATTTGCTCAACAAAATGCACATCTCATATAAAAATAAATGAGGAGCATGCACACACCA
CAAACACAAACAGGCATGCAGAAATACACATACACACTTCCCTCAATATAAACCCTTTGTGGCTCATAT
ATTTAAAAAGATGTAAAAAAAAGAGCTGAAGAAAATCATGTGTGATCTCTCAGCAGAATAGATTTATTA
TTTGTATTGCTTGCAGAATAAAGCCTATCCTTGAAAGCTCTGAATCATGGGCAAGAGGCTCAGTGGTA
TCTGGAGGACAGGGCACTGGCCACTGCAGTCACCATCTTCTGCCAGGAAGCCTGCACCTCAGGGGTG
AATTCTTTGCCAAAGTGAATGGCCAGCACGGTGACCAGCACGTTGCCCAGGAGCTGTGGGAGGAAGA
TAAGAGGTATGAACATGATTAGCAAAAGGGCCTAGCTTGGACTCAGAATAATCCAGCCTTATCCCAAC
CATAAAATAAAAGCAGAATGGTAGCTGGATTGTAGCTGCTATTAGCAATATGAAACCTCTTACATCAGT
TACAATTTATATGCAGAAATATTTATATGCAGAAATATTGCTATTGCCTTAACCCAGAAATTATCACTGT
TATTCTTTAGAATGGTGCAAAGAGGCATGATACATTGTATCATTATTGCCCTGAAAGAAAGAGATTAG
GGAAAGTATTAGAAATAAGATAAACAAAAAAGTATATTAAAAGAAGAAAGCATTTTTTAAAATTACAAA
TGCAAAATTACCCTGATTTGGTCAATATGTGTACCCTGTTACTTCTCCCCTTCCTATGACATGAACTTA
ACCATAGAAAAGAAGGGGAAAGAAAACATCAAGGGTCCCATAGACTCACCTTGAAGTTCTCAGGATC
CACATGCAGCTTGTCACAGTGCAGTTCACTCAGCTGGGCAAAGGTGCCCTTGAGATCATCCAGGTGC
TTTATGGCATCTCCCAAGGAAGTCAGCACCTTCTTGCCATGTGCCTTGACTTTGGGGTTGCCCATGAT
GGCAGAGGCAGAGGACAGGTTGCCAAAGCTGTCAAAGAACCTCTGGGTCCATGGGTAGACAACCAG
GAGCCTGTGAGATTGACAAGAACAGTTTGACAGTCAGAAGGTGCCACAAATCCTGAGAAGCAACCTG
GACTTTTGCCAGGCACAGGGTCCTTCCTTCCCTCCCTTGTCCTGGTCACCAGAGCCTACCTTCCCAGG
GTTTCTCCTCCAGCATCTTCCACATTCACCTTGTCCCACAGGCTTGTGATAGTAGCCTTGTCCTCCTCT
GTGAAATGACCCATGGTGTCTGTTTGAGGTTGCTAGTGAACACAGTTGTGTCAGAAGCAAATGTAAGC
AATAGATGGCTCTGCCCTGACTTTTATGCCCAGCCCTGGCTCCTGCCCTCCCTGCTCCTGGGAGTAGA
TTGGCCAACCCTAGGGTGTGGCTCCACAGGGTGAGGTCTAAGTGATGACAGCCGTACCTGTCCTTGG
CTCTTCTGGCACTGGCTTAGGAGTTGGACTTCAAACCCTCAGCCCTCCCTCTAAGATATATCTCTTGG
CCCCATACCATCAGTACAAATTGCTACTAAAAACATCCTCCTTTGCAAGTGTATTTACGACGGTATCGA
TGTATGTGAGCATGTGTCCTCTAACAGCACAGGCCTTTTGCCACCTAGCTGTCCAGGGGTGCCTTAAA
ATGGCAAACAAGGTTTGTTTTCTTTTCCTGTTTTCATGCCTTCCTCTTCCATATCCTTGTTTCATATTAAT
ACATGTGTATAGATCCTAAAAATCTATACACATGTATTAATAAAGCCTGATTCTGCCGCTTCTAGGTAT
AGAGGCCACCTGCAAGATAAATATTTGATTCACAATAACTAATCATTCTATGGCAATTGATAACAACAA
ATATATATATATATATATATATACGTATATGTGTATATATATATATATATTCAGGAAATAATATATTCTAG
AATATGTCACATTCTGTCTCAGGCATCCATTTTCTTTATGATGCCGTTTGAGGTGGAGTTTTAGTCAGG
TGGTCAGCTTCTCCTTTTTTTTGCCATCTGCCCTGTAAGCATCCTGCTGGGGACCCAGATAGGAGTCA
TCACTCTAGGCTGAGAACATCTGGGCACACACCCTAAGCCTCAGCATGACTCATCATGACTCAGCATT
GCTGTGCTTGAGCCAGAAGGTTTGCTTAGAAGGTTACACAGAACCAGAAGGCGGGGGTGGGGCACT
GACCCCGACAGGGGCCTGGCCAGAACTGCTCATGCTTGGACTATGGGAGGTCACTAATGGAGACAC
ACAGAAATGTAACAGGAACTAAGGGAATTCCGGTGCCCTGCTTAGGAGCTTAATCTTTAATGAAAGCT
AAGCTTTCATTAAAAAAAGTCTAACCAGCTGCATTCGACTTTGACTGCAGCAGCTGGTTAGAAGGTTC
TACTGGAGGAGGGTCCCAGCCCATTGCTAAATTAACATCAGGCTCTGAGACTGGCAGTATATCTCTAA
CAGTGGTTGATGCTATCTTCTGGAACTTGCCTGCTACATTGAGACCACTGACCCATACATAGGAAGCC
CATAGCTCTGTCCTGAACTGTTAGGCCACTGGTCCAGAGAGTGTGCATCTCCTTTGATCCTCATAATA
ACCCTATGAGATAGACACAATTATTACTCTTACTTTATAGATGATGATCCTGAAAACATAGGAGTCAAG
GCACTTGCCCCTAGCTGGGGGTATAGGGGAGCAGTCCCATGTAGTAGTAGAATGAAAAATGCTGCTA
TGCTGTGCCTCCCCCACCTTTCCCATGTCTGCCCTCTACTCATGGTCTATCTCTCCTGGCTCCTGGGA
GTCATGGACTCCACCCAGCACCACCAACCTGACCTAACCACCTATCTGAGCCTGCCAGCCTATAACCC
ATCTGGGCCCTGATAGCTGGTGGCCAGCCCTGACCCCACCCCACCCTCCCTGGAACCTCTGATAGAC
ACATCTGGCACACCAGCTCGCAAAGTCACCGTGAGGGTCTTGTGTTTGCTGAGTCAAAATTCCTTGAA
ATCCAAGTCCTTAGAGACTCCTGCTCCCAAATTTACAGTCATAGACTTCTTCATGGCTGTCTCCTTTAT
CCACAGAATGATTCCTTTGCTTCATTGCCCCATCCATCTGATCCTCCTCATCAGTGCAGCACAGGGCC
CATGAGCAGTAGCTGCAGAGTCTCACATAGGTCTGGCACTGCCTCTGACATGTCCGACCTTAGGCAA
ATGCTTGACTCTTCTGAGCTCGGATCCCTTGAGCTCAGGAGGTCAAGGCTGCAGTGAGACATGATCTT
GCCACTGCACTCCAGCCTGGACAGCAGAGTGAAACCTTGCCTCACGAAACAGAATACAAAAACAAAC
AAACAAAAAACTGCTCCGCAATGCGCTTCCTTGATGCTCTACCACATAGGTCTGGGTACTTTGTACAC
ATTATCTCATTGCTGTTCATAATTGTTAGATTAATTTTGTAATATTGATATTATTCCTAGAAAGCTGAGG
CCTCAAGATGATAACTTTTATTTTCTGGACTTGTAATAGCTTTCTCTTGTATTCACCATGTTGTAACTTT
CTTAGAGTAGTAACAATATAAAGTTATTGTGAGTTTTTGCAAACACAGCAAACACAACGACCCATATAG
ACATTGATGTGAAATTGTCTATTGTCAATTTATGGGAAAACAAGTATGTACTTTTTCTACTAAGCCATT
GAAACAGGAATAACAGAACAAGATTGAAAGAATACATTTTCCGAAATTACTTGAGTATTATACAAAGAC
AAGCACGTGGACCTGGGAGGAGGGTTATTGTCCATGACTGGTGTGTGGAGACAAATGCAGGTTTATA
ATAGATGGGATGGCATCTAGCGCAATGACTTTGCCATCACTTTTAGAGAGCTCTTGGGGGCCCCAGTA
CACAAGAGGGGACGCAGGGTATATGTAGACATCTCATTCTTTTTCTTAGTGTGAGAATAAGAATAGCC
ATGACCTGAGTTTATAGACAATGAGCCCTTTTCTCTCTCCCACTCAGCAGCTATGAGATGGCTTGCCCT
GCCTCTCTACTAGGCTGACTCACTCCAAGGCCCAGCAATGGGCAGGGCTCTGTCAGGGCTTTGATAG
CACTATCTGCAGAGCCAGGGCCGAGAAGGGGTGGACTCCAGAGACTCTCCCTCCCATTCCCGAGCAG
GGTTTGCTTATTTATGCATTTAAATGATATATTTATTTTAAAAGAAATAACAGGAGACTGCCCAGCCCT
GGCTGTGACATGGAAACTATGTAGAATATTTTGGGTTCCATTTTTTTTTCCTTCTTTCAGTTAGAGGAA
AAGGGGCTCACTGCACATACACTAGACAGAAAGTCAGGAGCTTTGAATCCAAGCCTGATCATTTCCAT
GTCATACTGAGAAAGTCCCCACCCTTCTCTGAGCCTCAGTTTCTCTTTTTATAAGTAGGAGTCTGGAGT
AAATGATTTCCAATGGCTCTCATTTCAATACAAAATTTCCGTTTATTAAATGCATGAGCTTCCGTTACTC
CAAGACTGAGAAGGAAATTGAACCTGAGACTCATTGACTGGCAAGATGTCCCCAGAGGCTCTCATTCA
GCAATAAAATTCTCACCTTCACCCAGGCCCACTAGTGTCAGATTTGCATGCGTTCGCGTATCGACGTG
CAGTATTTAGCATGCCCCACCCATCTGCAAGGCATTCTGGATAGTGTCAAAACAGCCGGAAATCAAGT
CCGTTTATCTCAAACTTTAGCATTTTGGGAATAAATGATATTTGCTATGCTGGTTAAATTAGATTTTAGT
TAAATTTCCTGCTGAAGCTCTAGTACGATAAGTAACTTGACCTAAGTGTAAAGTTGAGATTTCCTTCAG
GTTTATATAGCTTGTGCGCCGCCTGGGTACCTCAGGATATGCCCTTGACTATTTGTCCGACATAGTCA
AGGGCATATCCTTTTTTGTGCGGCCGCATCGATGCCGTAGTACCTTTAAGACCAATGACTTACAAGGC
AGCTGTAGATCTTAGCCACTTTTTAAAAGAAAAGGGGGGACTGGAAGGGCTAATTCACTCCCAAAGAA
GACAAGATCCCTGCAGGCATTCAAGGCCAGGCTGGATGTGGCTCTGGGCAGCCTGGGCTGCTGGTT
GATGACCCTGCACATAGCAGGGGGTTGGATCTGGATGAGCACTGTGCTCCTTTGCAACCCAGGCCGT
TCTATGATTCTGTCATTCTAAATCTCTCTTTCAGCCTAAAGCTTTTTCCCCGTATCCCCCCAGGTGTCTG
CAGGCTCAAAGAGCAGCGAGAAGCGTTCAGAGGAAAGCGATCCCGTGCCACCTTCCCCGTGCCCGG
GCTGTCCCCGCACGCTGCCGGCTCGGGGATGCGGGGGGAGCGCCGGACCGGAGCGGAGCCCCGG
GCGGCTCGCTGCTGCCCCCTAGCGGGGGAGGGACGTAATTACATCCCTGGGGGCTTTGGGGGGGG
GCTGTCCCCGTGAGCTCCCCAGATCTGCTTTTTGCCTGTACTGGGTCTCTCTGGTTAGACCAGATCTG
AGCCTGGGAGCTCTCTGGCTAACTAGGGAACCCACTGCTTAAGCCTCAATAAAGCTTCAGCTGCTCGA
GCTAGCAGATCTTTTTCCCTCTGCCAAAAATTATGGGGACATCATGAAGCCCCTTGAGCATCTGACTT
CTGGCTAATAAAGGAAATTTATTTTCATTGCAATAGTGTGTTGGAATTTTTTGTGTCTCTCACTCGGAA
GGACATATGGGAGGGCAAATCATTTAAAACATCAGAATGAGTATTTGGTTTAGAGTTTGGCAACATAT
GCCCATATGCTGGCTGCCATGAACAAAGGTTGGCTATAAAGAGGTCATCAGTATATGAAACAGCCCC
CTGCTGTCCATTCCTTATTCCATAGAAAAGCCTTGACTTGAGGTTAGATTTTTTTTATATTTTGTTTTGT
GTTATTTTTTTCTTTAACATCCCTAAAATTTTCCTTACATGTTTTACTAGCCAGATTTTTCCTCCTCTCCT
GACTACTCCCAGTCATAGCTGTCCCTCTTCTCTTATGGAGATCCCTCGACCTGCAGCCCAAGCTTGGC
GTAATCATGGTCATAGCTGTTTCCTGTGTGAAATTGTTATCCGCTCACAATTCCACACAACATACGAGC
CGGAAGCATAAAGTGTAAAGCCTGGGGTGCCTAATGAGTGAGCTAACTCACATTAATTGCGTTGCGC
TCACTGCCCGCTTTCCAGTCGGGAAACCTGTCGTGCCAGCGGATCCGCATCTCAATTAGTCAGCAACC
ATAGTCCCGCCCCTAACTCCGCCCATCCCGCCCCTAACTCCGCCCAGTTCCGCCCATTCTCCGCCCCA
TGGCTGACTAATTTTTTTTATTTATGCAGAGGCCGAGGCCGCCTCGGCCTCTGAGCTATTCCAGAAGT
AGTGAGGAGGCTTTTTTGGAGGCCTAGGCTTTTGCAAAAAGCTGTCGACTGCAGAGGCCTGCATGCA
AGCTTGGCGTAATCATGGTCATAGCTGTTTCCTGTGTGAAATTGTTATCCGCTCACAATTCCACACAAC
ATACGAGCCGGAAGCATAAAGTGTAAAGCCTGGGGTGCCTAATGAGTGAGCTAACTCACATTAATTG
CGTTGCGCTCACTGCCCGCTTTCCAGTCGGGAAACCTGTCGTGCCAGCTGCATTAATGAATCGGCCA
ACGCGCGGGGAGAGGCGGTTTGCGTATTGGGCGCTCTTCCGCTTCCTCGCTCACTGACTCGCTGCGC
TCGGTCGTTCGGCTGCGGCGAGCGGTATCAGCTCACTCAAAGGCGGTAATACGGTTATCCACAGAAT
CAGGGGATAACGCAGGAAAGAACATGTGAGCAAAAGGCCAGCAAAAGGCCAGGAACCGTAAAAAGG
CCGCGTTGCTGGCGTTTTTCCATAGGCTCCGCCCCCCTGACGAGCATCACAAAAATCGACGCTCAAGT
CAGAGGTGGCGAAACCCGACAGGACTATAAAGATACCAGGCGTTTCCCCCTGGAAGCTCCCTCGTGC
GCTCTCCTGTTCCGACCCTGCCGCTTACCGGATACCTGTCCGCCTTTCTCCCTTCGGGAAGCGTGGCG
CTTTCTCATAGCTCACGCTGTAGGTATCTCAGTTCGGTGTAGGTCGTTCGCTCCAAGCTGGGCTGTGT
GCACGAACCCCCCGTTCAGCCCGACCGCTGCGCCTTATCCGGTAACTATCGTCTTGAGTCCAACCCG
GTAAGACACGACTTATCGCCACTGGCAGCAGCCACTGGTAACAGGATTAGCAGAGCGAGGTATGTAG
GCGGTGCTACAGAGTTCTTGAAGTGGTGGCCTAACTACGGCTACACTAGAAGAACAGTATTTGGTATC
TGCGCTCTGCTGAAGCCAGTTACCTTCGGAAAAAGAGTTGGTAGCTCTTGATCCGGCAAACAAACCAC
CGCTGGTAGCGGTGGTTTTTTTGTTTGCAAGCAGCAGATTACGCGCAGAAAAAAAGGATCTCAAGAA
GATCCTTTGATCTTTTCTACGGGGTCTGACGCTCAGTGGAACGAAAACTCACGTTAAGGGATTTTGGT
CATGAGATTATCAAAAAGGATCTTCACCTAGATCCTTTTAAATTAAAAATGAAGTTTTAAATCAATCTAA
AGTATATATGAGTAAACTTGGTCTGACAGTTACCAATGCTTAATCAGTGAGGCACCTATCTCAGCGAT
CTGTCTATTTCGTTCATCCATAGTTGCCTGACTCCCCGTCGTGTAGATAACTACGATACGGGAGGGCT
TACCATCTGGCCCCAGTGCTGCAATGATACCGCGAGACCCACGCTCACCGGCTCCAGATTTATCAGC
AATAAACCAGCCAGCCGGAAGGGCCGAGCGCAGAAGTGGTCCTGCAACTTTATCCGCCTCCATCCAG
TCTATTAATTGTTGCCGGGAAGCTAGAGTAAGTAGTTCGCCAGTTAATAGTTTGCGCAACGTTGTTGC
CATTGCTACAGGCATCGTGGTGTCACGCTCGTCGTTTGGTATGGCTTCATTCAGCTCCGGTTCCCAAC
GATCAAGGCGAGTTACATGATCCCCCATGTTGTGCAAAAAAGCGGTTAGCTCCTTCGGTCCTCCGATC
GTTGTCAGAAGTAAGTTGGCCGCAGTGTTATCACTCATGGTTATGGCAGCACTGCATAATTCTCTTAC
TGTCATGCCATCCGTAAGATGCTTTTCTGTGACTGGTGAGTACTCAACCAAGTCATTCTGAGAATAGT
GTATGCGGCGACCGAGTTGCTCTTGCCCGGCGTCAATACGGGATAATACCGCGCCACATAGCAGAAC
TTTAAAAGTGCTCATCATTGGAAAACGTTCTTCGGGGCGAAAACTCTCAAGGATCTTACCGCTGTTGA
GATCCAGTTCGATGTAACCCACTCGTGCACCCAACTGATCTTCAGCATCTTTTACTTTCACCAGCGTTT
CTGGGTGAGCAAAAACAGGAAGGCAAAATGCCGCAAAAAAGGGAATAAGGGCGACACGGAAATGTT
GAATACTCATACTCTTCCTTTTTCAATATTATTGAAGCATTTATCAGGGTTATTGTCTCATGAGCGGATA
CATATTTGAATGTATTTAGAAAAATAAACAAATAGGGGTTCCGCGCACATTTCCCCGAAAAGTGCCAC
CTGACGTCTAAGAAACCATTATTATCATGACATTAACCTATAAAAATAGGCGTATCACGAGGCCCTTTC
GTCTCGCGCGTTTCGGTGATGACGGTGAAAACCTCTGACACATGCAGCTCCCGGAGACGGTCACAGC
TTGTCTGTAAGCGGATGCCGGGAGCAGACAAGCCCGTCAGGGCGCGTCAGCGGGTGTTGGCGGGTG
TCGGGGCTGGCTTAACTATGCGGCATCAGAGCAGATTGTACTGAGAGTGCACCATATGCGGTGTGAA
ATACCGCACAGATGCGTAAGGAGAAAATACCGCATCAGGCGCCATTCGCCATTCAGGCTGCGCAACT
GTTGGGAAGGGCGATCGGTGCGGGCCTCTTCGCTATTACGCCAGCTGGCGAAAGGGGGATGTGCTG
CAAGGCGATTAAGTTGGGTAACGCCAGGGTTTTCCCAGTCACGACGTTGTAAAACGACGGCCAGTGA
ATTC
pCalH20 TL20d rGbGM 7SKsh734 (SEQ ID NO: 108)
GGCCGCCTCGGCCAAACAGCCCTTGAGTTTACCACTCCCTATCAGTGATAGAGAAAAGTGAAAGTCG
AGTTTACCACTCCCTATCAGTGATAGAGAAAAGTGAAAGTCGAGTTTACCACTCCCTATCAGTGATAG
AGAAAAGTGAAAGTCGAGTTTACCACTCCCTATCAGTGATAGAGAAAAGTGAAAGTCGAGTTTACCAG
TCCCTATCAGTGATAGAGAAAAGTGAAAGTCGAGTTTACCACTCCCTATCAGTGATAGAGAAAAGTGA
AAGTCGAGTTTACCACTCCCTATCAGTGATAGAGAAAAGTGAAAGTCGAGCTCGCCATGGGAGGCGT
GGCCTGGGCGGGACTGGGGAGTGGCGAGCCCTCAGATCCTGCATATAAGCAGCTGCTTTTTGCCTGT
ACTGGGTCTCTCTGGTTAGACCAGATCTGAGCCTGGGAGCTCTCTGGCTAACTAGGGAACCCACTGC
TTAAGCCTCAATAAAGCTTGCCTTGAGTGCTTCAAGTAGTGTGTGCCCGTCTGTTGTGTGACTCTGGT
AACTAGAGATCCCTCAGACCCTTTTAGTCAGTGTGGAAAATCTCTAGCAGTGGCGCCCGAACAGGGA
CTTGAAAGCGAAAGGGAAACCAGAGGAGCTCTCTCGACGCAGGACTCGGCTTGCTGAAGCGCGCAC
GGCAAGAGGCGAGGGGCGGCGACTGGTGAGTACGCCAAAAATTTTGACTAGCGGAGGCTAGAAGGA
GAGAGATGGGTGCGAGAGCGTCAGTATTAAGCGGGGGAGAATTAGATCGCGATGGGAAAAAATTCG
GTTAAGGCCAGGGGGAAAGAAAAAATATAAATTAAAACATATAGTATGGGCAAGCAGGGAGCTAGAA
CGATTCGCAGTTAATACTGGCCTGTTAGAAACATCAGAAGGCTGTAGACAAATACTGGGACAGCTACA
ACCATCCCTTCAGACAGGATCAGAAGAACTTAGATCATTATATAATACAGTAGCAACCCTCTATTGTGT
GCATCAAAGGATAGAGATAAAAGACACCAAGGAAGCTTTAGACAAGATAGAGGAAGAGCAAAACAAA
AGTAAGAAAAAAGCACAGCAAGCAGCAGGATCTTCAGACCTGGAAATTCCCTACAATCCCCAAAGTCA
AGGAGTAGTAGAATCTATGAATAAAGAATTAAAGAAAATTATAGGACAGGTAAGAGATCAGGCTGAAC
ATCTTAAGACAGCAGTACAAATGGCAGTATTCATCCACAATTTTAAAAGAAAAGGGGGGATTGGGGG
GTACAGTGCAGGGGAAAGAATAGTAGACATAATAGCAACAGACATACAAACTAAAGAATTACAAAAAC
AAATTACAAAAATTCAAAATTTTCGGGTTTATTACAGGGACAGCAGAAATCCACTTTGGAAAGGACCA
GCAAAGCTCCTCTGGAAAGGTGAAGGGGCAGTAGTAATACAAGATAATAGTGACATAAAAGTAGTGC
CAAGAAGAAAAGCAAAGATCATTAGGGATTATGGAAAACAGATGGCAGGTGATGATTGTGTGGCAAG
TAGACAGGATGAGGATTAGAACATGGAAAAGTTTAGTAAAACACCATAAGGAGGAGATATGAGGGAC
AATTGGAGAAGTGAATTATATAAATATAAAGTAGTAAAAATTGAACCATTAGGAGTAGCACCCACCAA
GGCAAAGAGAAGAGTGGTGCAGAGAGAAAAAAGAGCAGTGGGAATAGGAGCTTTGTTCCTTGGGTT
CTTGGGAGCAGCAGGAAGCACTATGGGCGCAGCGTCAATGACGCTGACGGTACAGGCCAGACAATT
ATTGTCTGGTATAGTGCAGCAGCAGAACAATTTGCTGAGGGCTATTGAGGCGCAACAGCATCTGTTG
CAACTCACAGTCTGGGGCATCAAGCAGCTCCAGGCAAGAATCCTGGCTGTGGAAAGATACCTAAAGG
ATCAACAGCTCCTGGGGATTTGGGGTTGCTCTGGAAAACTCATTTGCACCACTGCTGTGCCTTGGAAT
GCTAGTTGGAGTAATAAATCTCTGGAACAGATTTGGAATCACACGACCTGGATGGAGTGGGACAGAG
AAATTAACAATTACACAAGCTTAATACACTCCTTAATTGAAGAATCGCAAAACCAGCAAGAAAAGAATG
AACAAGAATTATTGGAATTAGATAAATGGGCAAGTTTGTGGAATTGGTTTAACATAACAAATTGGCTGT
GGTATATAAAATTATTCATAATGATAGTAGGAGGCTTGGTAGGTTTAAGAATAGTTTTTGCTGTACTTT
CTATAGTGAATAGAGTTAGGCAGGGATATTCACCATTATCGTTTCAGACCCACCTCCCAACCCCGAGG
GGACCGAGCTCAAGCTTCGAACGCGTGGGGATCCTCTAGAGTCGAGCTCGCGAGGATCATCACCGG
TGCTAGCCGGAGCCAGAAGCACCATAAGGGACATGATAAGGGAGCCAGCAGACCTCTGATCTCTTCC
TGAATGCTAATCTTAAACATCCTGAGGAAGAATGGGACTTCCATTTGGGGTGGGCCTATGATAGGGTA
ATAAGACAGTAGTGAATATCAAGCTACAAAAAGCCCCCTTTCAAATTCTTCTCAGTCCTAACTTTTCAT
ACTAAGCCCAGTCCTTCCAAAGCAGACTGTGAAAGAGTGATAGTTCCGGGAGACTAGCACCGGCTAG
CCGAGCTTGGAACACTTTCCCTTCATTAAGAACCATCCTTGCTACTCAGCTGCAATCAATCCAGCCCCC
AGGTCTTCACTGAACCTTTTCCCATCTCTTCCAAAACATCTGTTTCTGAGAAGTCCTGTCCTATAGAGG
TCTTTCTTCCCACCGGATTTCTCCTACACCATTTACTCCCACTTGCAGAACTCCCGTGTACAAGTGTCT
TTACTGCTTTTATTTGCTCAACAAAATGCACATCTCATATAAAAATAAATGAGGAGCATGCACACACCA
CAAACACAAACAGGCATGCAGAAATACACATACACACTTCCCTCAATATAAACCCTTTGTGGCTCATAT
ATTTAAAAAGATGTAAAAAAAAGAGCTGAAGAAAATCATGTGTGATCTCTCAGCAGAATAGATTTATTA
TTTGTATTGCTTGCAGAATAAAGCCTATCCTTGAAAGCTCTGAATCATGGGCAAGAGGCTCAGTGGTA
TCTGGAGGACAGGGCACTGGCCACTGCAGTCACCATCTTCTGCCAGGAAGCCTGCACCTCAGGGGTG
AATTCTTTGCCAAAGTGAATGGCCAGCACGGTGACCAGCACGTTGCCCAGGAGCTGTGGGAGGAAGA
TAAGAGGTATGAACATGATTAGCAAAAGGGCCTAGCTTGGACTCAGAATAATCCAGCCTTATCCCAAC
CATAAAATAAAAGCAGAATGGTAGCTGGATTGTAGCTGCTATTAGCAATATGAAACCTCTTACATCAGT
TACAATTTATATGCAGAAATATTTATATGCAGAAATATTGCTATTGCCTTAACCCAGAAATTATCACTGT
TATTCTTTAGAATGGTGCAAAGAGGCATGATACATTGTATCATTATTGCCCTGAAAGAAAGAGATTAG
GGAAAGTATTAGAAATAAGATAAACAAAAAAGTATATTAAAAGAAGAAAGCATTTTTTAAAATTACAAA
TGCAAAATTACCCTGATTTGGTCAATATGTGTACCCTGTTACTTCTCCCCTTCCTATGACATGAACTTA
ACCATAGAAAAGAAGGGGAAAGAAAACATCAAGGGTCCCATAGACTCACCTTGAAGTTCTCAGGATC
CACATGCAGCTTGTCACAGTGCAGTTCACTCAGCTGGGCAAAGGTGCCCTTGAGATCATCCAGGTGC
TTTATGGCATCTCCCAAGGAAGTCAGCACCTTCTTGCCATGTGCCTTGACTTTGGGGTTGCCCATGAT
GGCAGAGGCAGAGGACAGGTTGCCAAAGCTGTCAAAGAACCTCTGGGTCCATGGGTAGACAACCAG
GAGCCTGTGAGATTGACAAGAACAGTTTGACAGTCAGAAGGTGCCACAAATCCTGAGAAGCAACCTG
GACTTTTGCCAGGCACAGGGTCCTTCCTTCCCTCCCTTGTCCTGGTCACCAGAGCCTACCTTCCCAGG
GTTTCTCCTCCAGCATCTTCCACATTCACCTTGTCCCACAGGCTTGTGATAGTAGCCTTGTCCTCCTCT
GTGAAATGACCCATGGTGTCTGTTTGAGGTTGCTAGTGAACACAGTTGTGTCAGAAGCAAATGTAAGC
AATAGATGGCTCTGCCCTGACTTTTATGCCCAGCCCTGGCTCCTGCCCTCCCTGCTCCTGGGAGTAGA
TTGGCCAACCCTAGGGTGTGGCTCCACAGGGTGAGGTCTAAGTGATGACAGCCGTACCTGTCCTTGG
CTCTTCTGGCACTGGCTTAGGAGTTGGACTTCAAACCCTCAGCCCTCCCTCTAAGATATATCTCTTGG
CCCCATACCATCAGTACAAATTGCTACTAAAAACATCCTCCTTTGCAAGTGTATTTACGACGGTATCGA
TGTATGTGAGCATGTGTCCTCTAACAGCACAGGCCTTTTGCCACCTAGCTGTCCAGGGGTGCCTTAAA
ATGGCAAACAAGGTTTGTTTTCTTTTCCTGTTTTCATGCCTTCCTCTTCCATATCCTTGTTTCATATTAAT
ACATGTGTATAGATCCTAAAAATCTATACACATGTATTAATAAAGCCTGATTCTGCCGCTTCTAGGTAT
AGAGGCCACCTGCAAGATAAATATTTGATTCACAATAACTAATCATTCTATGGCAATTGATAACAACAA
ATATATATATATATATATATATACGTATATGTGTATATATATATATATATTCAGGAAATAATATATTCTAG
AATATGTCACATTCTGTCTCAGGCATCCATTTTCTTTATGATGCCGTTTGAGGTGGAGTTTTAGTCAGG
TGGTCAGCTTCTCCTTTTTTTTGCCATCTGCCCTGTAAGCATCCTGCTGGGGACCCAGATAGGAGTCA
TCACTCTAGGCTGAGAACATCTGGGCACACACCCTAAGCCTCAGCATGACTCATCATGACTCAGCATT
GCTGTGCTTGAGCCAGAAGGTTTGCTTAGAAGGTTACACAGAACCAGAAGGCGGGGGTGGGGCACT
GACCCCGACAGGGGCCTGGCCAGAACTGCTCATGCTTGGACTATGGGAGGTCACTAATGGAGACAC
ACAGAAATGTAACAGGAACTAAGGGAATTCCGGTGCCCTGCTTAGGAGCTTAATCTTTAATGAAAGCT
AAGCTTTCATTAAAAAAAGTCTAACCAGCTGCATTCGACTTTGACTGCAGCAGCTGGTTAGAAGGTTC
TACTGGAGGAGGGTCCCAGCCCATTGCTAAATTAACATCAGGCTCTGAGACTGGCAGTATATCTCTAA
CAGTGGTTGATGCTATCTTCTGGAACTTGCCTGCTACATTGAGACCACTGACCCATACATAGGAAGCC
CATAGCTCTGTCCTGAACTGTTAGGCCACTGGTCCAGAGAGTGTGCATCTCCTTTGATCCTCATAATA
ACCCTATGAGATAGACACAATTATTACTCTTACTTTATAGATGATGATCCTGAAAACATAGGAGTCAAG
GCACTTGCCCCTAGCTGGGGGTATAGGGGAGCAGTCCCATGTAGTAGTAGAATGAAAAATGCTGCTA
TGCTGTGCCTCCCCCACCTTTCCCATGTCTGCCCTCTACTCATGGTCTATCTCTCCTGGCTCCTGGGA
GTCATGGACTCCACCCAGCACCACCAACCTGACCTAACCACCTATCTGAGCCTGCCAGCCTATAACCC
ATCTGGGCCCTGATAGCTGGTGGCCAGCCCTGACCCCACCCCACCCTCCCTGGAACCTCTGATAGAC
ACATCTGGCACACCAGCTCGCAAAGTCACCGTGAGGGTCTTGTGTTTGCTGAGTCAAAATTCCTTGAA
ATCCAAGTCCTTAGAGACTCCTGCTCCCAAATTTACAGTCATAGACTTCTTCATGGCTGTCTCCTTTAT
CCACAGAATGATTCCTTTGCTTCATTGCCCCATCCATCTGATCCTCCTCATCAGTGCAGCACAGGGCC
CATGAGCAGTAGCTGCAGAGTCTCACATAGGTCTGGCACTGCCTCTGACATGTCCGACCTTAGGCAA
ATGCTTGACTCTTCTGAGCTCGGATCCCTTGAGCTCAGGAGGTCAAGGCTGCAGTGAGACATGATCTT
GCCACTGCACTCCAGCCTGGACAGCAGAGTGAAACCTTGCCTCACGAAACAGAATACAAAAACAAAC
AAACAAAAAACTGCTCCGCAATGCGCTTCCTTGATGCTCTACCACATAGGTCTGGGTACTTTGTACAC
ATTATCTCATTGCTGTTCATAATTGTTAGATTAATTTTGTAATATTGATATTATTCCTAGAAAGCTGAGG
CCTCAAGATGATAACTTTTATTTTCTGGACTTGTAATAGCTTTCTCTTGTATTCACCATGTTGTAACTTT
CTTAGAGTAGTAACAATATAAAGTTATTGTGAGTTTTTGCAAACACAGCAAACACAACGACCCATATAG
ACATTGATGTGAAATTGTCTATTGTCAATTTATGGGAAAACAAGTATGTACTTTTTCTACTAAGCCATT
GAAACAGGAATAACAGAACAAGATTGAAAGAATACATTTTCCGAAATTACTTGAGTATTATACAAAGAC
AAGCACGTGGACCTGGGAGGAGGGTTATTGTCCATGACTGGTGTGTGGAGACAAATGCAGGTTTATA
ATAGATGGGATGGCATCTAGCGCAATGACTTTGCCATCACTTTTAGAGAGCTCTTGGGGGCCCCAGTA
CACAAGAGGGGACGCAGGGTATATGTAGACATCTCATTCTTTTTCTTAGTGTGAGAATAAGAATAGCC
ATGACCTGAGTTTATAGACAATGAGCCCTTTTCTCTCTCCCACTCAGCAGCTATGAGATGGCTTGCCCT
GCCTCTCTACTAGGCTGACTCACTCCAAGGCCCAGCAATGGGCAGGGCTCTGTCAGGGCTTTGATAG
CACTATCTGCAGAGCCAGGGCCGAGAAGGGGTGGACTCCAGAGACTCTCCCTCCCATTCCCGAGCAG
GGTTTGCTTATTTATGCATTTAAATGATATATTTATTTTAAAAGAAATAACAGGAGACTGCCCAGCCCT
GGCTGTGACATGGAAACTATGTAGAATATTTTGGGTTCCATTTTTTTTTCCTTCTTTCAGTTAGAGGAA
AAGGGGCTCACTGCACATACACTAGACAGAAAGTCAGGAGCTTTGAATCCAAGCCTGATCATTTCCAT
GTCATACTGAGAAAGTCCCCACCCTTCTCTGAGCCTCAGTTTCTCTTTTTATAAGTAGGAGTCTGGAGT
AAATGATTTCCAATGGCTCTCATTTCAATACAAAATTTCCGTTTATTAAATGCATGAGCTTCCGTTACTC
CAAGACTGAGAAGGAAATTGAACCTGAGACTCATTGACTGGCAAGATGTCCCCAGAGGCTCTCATTCA
GCAATAAAATTCTCACCTTCACCCAGGCCCACTAGTGTCAGATTTGCATGCGTTCGCGTATCGACGTG
CAGTATTTAGCATGCCCCACCCATCTGCAAGGCATTCTGGATAGTGTCAAAACAGCCGGAAATCAAGT
CCGTTTATCTCAAACTTTAGCATTTTGGGAATAAATGATATTTGCTATGCTGGTTAAATTAGATTTTAGT
TAAATTTCCTGCTGAAGCTCTAGTACGATAAGTAACTTGACCTAAGTGTAAAGTTGAGATTTCCTTCAG
GTTTATATAGCTTGTGCGCCGCCTGGGTACCTCAGGATATGCCCTTGACTATTTGTCCGACATAGTCA
AGGGCATATCCTTTTTTGTGCGGCCGCATCGATGCCGTAGTACCTTTAAGACCAATGACTTACAAGGC
AGCTGTAGATCTTAGCCACTTTTTAAAAGAAAAGGGGGGACTGGAAGGGCTAATTCACTCCCAAAGAA
GACAAGATAGATCTGCTTTTTGCCTGTACTGGGTCTCTCTGGTTAGACCAGATCTGAGCCTGGGAGCT
CTCTGGCTAACTAGGGAACCCACTGCTTAAGCCTCAATAAAGCTTCAGCTGCTCGAGCTAGCAGATCT
TTTTCCCTCTGCCAAAAATTATGGGGACATCATGAAGCCCCTTGAGCATCTGACTTCTGGCTAATAAA
GGAAATTTATTTTCATTGCAATAGTGTGTTGGAATTTTTTGTGTCTCTCACTCGGAAGGACATATGGGA
GGGCAAATCATTTAAAACATCAGAATGAGTATTTGGTTTAGAGTTTGGCAACATATGCCCATATGCTG
GCTGCCATGAACAAAGGTTGGCTATAAAGAGGTCATCAGTATATGAAACAGCCCCCTGCTGTCCATTC
CTTATTCCATAGAAAAGCCTTGACTTGAGGTTAGATTTTTTTTATATTTTGTTTTGTGTTATTTTTTTCTT
TAACATCCCTAAAATTTTCCTTACATGTTTTACTAGCCAGATTTTTCCTCCTCTCCTGACTACTCCCAGT
CATAGCTGTCCCTCTTCTCTTATGGAGATCCCTCGACCTGCAGCCCAAGCTTGGCGTAATCATGGTCA
TAGCTGTTTCCTGTGTGAAATTGTTATCCGCTCACAATTCCACACAACATACGAGCCGGAAGCATAAA
GTGTAAAGCCTGGGGTGCCTAATGAGTGAGCTAACTCACATTAATTGCGTTGCGCTCACTGCCCGCTT
TCCAGTCGGGAAACCTGTCGTGCCAGCGGATCCGCATCTCAATTAGTCAGCAACCATAGTCCCGCCC
CTAACTCCGCCCATCCCGCCCCTAACTCCGCCCAGTTCCGCCCATTCTCCGCCCCATGGCTGACTAAT
TTTTTTTATTTATGCAGAGGCCGAGGCCGCCTCGGCCTCTGAGCTATTCCAGAAGTAGTGAGGAGGCT
TTTTTGGAGGCCTAGGCTTTTGCAAAAAGCTGTCGACTGCAGAGGCCTGCATGCAAGCTTGGCGTAA
TCATGGTCATAGCTGTTTCCTGTGTGAAATTGTTATCCGCTCACAATTCCACACAACATACGAGCCGG
AAGCATAAAGTGTAAAGCCTGGGGTGCCTAATGAGTGAGCTAACTCACATTAATTGCGTTGCGCTCAC
TGCCCGCTTTCCAGTCGGGAAACCTGTCGTGCCAGCTGCATTAATGAATCGGCCAACGCGCGGGGAG
AGGCGGTTTGCGTATTGGGCGCTCTTCCGCTTCCTCGCTCACTGACTCGCTGCGCTCGGTCGTTCGG
CTGCGGCGAGCGGTATCAGCTCACTCAAAGGCGGTAATACGGTTATCCACAGAATCAGGGGATAACG
CAGGAAAGAACATGTGAGCAAAAGGCCAGCAAAAGGCCAGGAACCGTAAAAAGGCCGCGTTGCTGG
CGTTTTTCCATAGGCTCCGCCCCCCTGACGAGCATCACAAAAATCGACGCTCAAGTCAGAGGTGGCG
AAACCCGACAGGACTATAAAGATACCAGGCGTTTCCCCCTGGAAGCTCCCTCGTGCGCTCTCCTGTTC
CGACCCTGCCGCTTACCGGATACCTGTCCGCCTTTCTCCCTTCGGGAAGCGTGGCGCTTTCTCATAGC
TCACGCTGTAGGTATCTCAGTTCGGTGTAGGTCGTTCGCTCCAAGCTGGGCTGTGTGCACGAACCCC
CCGTTCAGCCCGACCGCTGCGCCTTATCCGGTAACTATCGTCTTGAGTCCAACCCGGTAAGACACGA
CTTATCGCCACTGGCAGCAGCCACTGGTAACAGGATTAGCAGAGCGAGGTATGTAGGCGGTGCTACA
GAGTTCTTGAAGTGGTGGCCTAACTACGGCTACACTAGAAGAACAGTATTTGGTATCTGCGCTCTGCT
GAAGCCAGTTACCTTCGGAAAAAGAGTTGGTAGCTCTTGATCCGGCAAACAAACCACCGCTGGTAGC
GGTGGTTTTTTTGTTTGCAAGCAGCAGATTACGCGCAGAAAAAAAGGATCTCAAGAAGATCCTTTGAT
CTTTTCTACGGGGTCTGACGCTCAGTGGAACGAAAACTCACGTTAAGGGATTTTGGTCATGAGATTAT
CAAAAAGGATCTTCACCTAGATCCTTTTAAATTAAAAATGAAGTTTTAAATCAATCTAAAGTATATATGA
GTAAACTTGGTCTGACAGTTACCAATGCTTAATCAGTGAGGCACCTATCTCAGCGATCTGTCTATTTCG
TTCATCCATAGTTGCCTGACTCCCCGTCGTGTAGATAACTACGATACGGGAGGGCTTACCATCTGGCC
CCAGTGCTGCAATGATACCGCGAGACCCACGCTCACCGGCTCCAGATTTATCAGCAATAAACCAGCC
AGCCGGAAGGGCCGAGCGCAGAAGTGGTCCTGCAACTTTATCCGCCTCCATCCAGTCTATTAATTGTT
GCCGGGAAGCTAGAGTAAGTAGTTCGCCAGTTAATAGTTTGCGCAACGTTGTTGCCATTGCTACAGG
CATCGTGGTGTCACGCTCGTCGTTTGGTATGGCTTCATTCAGCTCCGGTTCCCAACGATCAAGGCGAG
TTACATGATCCCCCATGTTGTGCAAAAAAGCGGTTAGCTCCTTCGGTCCTCCGATCGTTGTCAGAAGT
AAGTTGGCCGCAGTGTTATCACTCATGGTTATGGCAGCACTGCATAATTCTCTTACTGTCATGCCATCC
GTAAGATGCTTTTCTGTGACTGGTGAGTACTCAACCAAGTCATTCTGAGAATAGTGTATGCGGCGACC
GAGTTGCTCTTGCCCGGCGTCAATACGGGATAATACCGCGCCACATAGCAGAACTTTAAAAGTGCTCA
TCATTGGAAAACGTTCTTCGGGGCGAAAACTCTCAAGGATCTTACCGCTGTTGAGATCCAGTTCGATG
TAACCCACTCGTGCACCCAACTGATCTTCAGCATCTTTTACTTTCACCAGCGTTTCTGGGTGAGCAAAA
ACAGGAAGGCAAAATGCCGCAAAAAAGGGAATAAGGGCGACACGGAAATGTTGAATACTCATACTCT
TCCTTTTTCAATATTATTGAAGCATTTATCAGGGTTATTGTCTCATGAGCGGATACATATTTGAATGTAT
TTAGAAAAATAAACAAATAGGGGTTCCGCGCACATTTCCCCGAAAAGTGCCACCTGACGTCTAAGAAA
CCATTATTATCATGACATTAACCTATAAAAATAGGCGTATCACGAGGCCCTTTCGTCTCGCGCGTTTCG
GTGATGACGGTGAAAACCTCTGACACATGCAGCTCCCGGAGACGGTCACAGCTTGTCTGTAAGCGGA
TGCCGGGAGCAGACAAGCCCGTCAGGGCGCGTCAGCGGGTGTTGGCGGGTGTCGGGGCTGGCTTA
ACTATGCGGCATCAGAGCAGATTGTACTGAGAGTGCACCATATGCGGTGTGAAATACCGCACAGATG
CGTAAGGAGAAAATACCGCATCAGGCGCCATTCGCCATTCAGGCTGCGCAACTGTTGGGAAGGGCGA
TCGGTGCGGGCCTCTTCGCTATTACGCCAGCTGGCGAAAGGGGGATGTGCTGCAAGGCGATTAAGTT
GGGTAACGCCAGGGTTTTCCCAGTCACGACGTTGTAAAACGACGGCCAGTGAATTCGGCCGCCTCGG
CCAAACAGCCCTTGAGTTTACCACTCCCTATCAGTGATAGAGAAAAGTGAAAGTCGAGTTTACCACTC
CCTATCAGTGATAGAGAAAAGTGAAAGTCGAGTTTACCACTCCCTATCAGTGATAGAGAAAAGTGAAA
GTCGAGTTTACCACTCCCTATCAGTGATAGAGAAAAGTGAAAGTCGAGTTATTTCCCCGAAAAGTGCC
ACCTGACGTCTAAGAAACCATTATTATCATGACATTACCTATAAAAATAGGCGTATCACGAGGCCCTTT
CGTCTCGCGCGTTTCGGTGATGACGGTGAAAACCTCTGACACATGCAGCTCCCGGAGACGGTCACAG
CTTGTCTGTAAGCGGATGCCGGGAGCAGACAAGCCCGTCAGGGCGCGTCAGCGGGTGTTGGCGGGT
GTCGGGGCTGGCTTAACTATGCGGCATCAGAGCAGATTGTACTGAGAGTGCACCATATGCGGTGTGA
AATACCGCACAGATGCGTAAGGAGAAAATACCGCATCAGGCGCCATTCGCCATTCAGGCTGCGCAAC
TGTTGGGAAGGGCGATCGGTGCGGGCCTCTTCGCTATTACGCCAGCTGGCGAAAGGGGGATGTGCT
GCAAGGCGATTAAGTTGGGTAACGCCAGGGTTTTCCCAGTCACGACGTTGTAAAACGACGGCCAGTG
AATTC
pCalH21 TL20d rGbGM G3320A 7SKsh734 (SEQ ID NO: 109)
GGCCGCCTCGGCCAAACAGCCCTTGAGTTTACCACTCCCTATCAGTGATAGAGAAAAGTGAAAGTCG
AGTTTACCACTCCCTATCAGTGATAGAGAAAAGTGAAAGTCGAGTTTACCACTCCCTATCAGTGATAG
AGAAAAGTGAAAGTCGAGTTTACCACTCCCTATCAGTGATAGAGAAAAGTGAAAGTCGAGTTTACCAG
TCCCTATCAGTGATAGAGAAAAGTGAAAGTCGAGTTTACCACTCCCTATCAGTGATAGAGAAAAGTGA
AAGTCGAGTTTACCACTCCCTATCAGTGATAGAGAAAAGTGAAAGTCGAGCTCGCCATGGGAGGCGT
GGCCTGGGGGGACTGGGGAGTGGCGAGCCCTCAGATCCTGCATATAAGCAGCTGCTTTTTGCCTGT
ACTGGGTCTCTCTGGTTAGACCAGATCTGAGCCTGGGAGCTCTCTGGCTAACTAGGGAACCCACTGC
TTAAGCCTCAATAAAGCTTGCCTTGAGTGCTTCAAGTAGTGTGTGCCCGTCTGTTGTGTGACTCTGGT
AACTAGAGATCCCTCAGACCCTTTTAGTCAGTGTGGAAAATCTCTAGCAGTGGCGCCCGAACAGGGA
CTTGAAAGCGAAAGGGAAACCAGAGGAGCTCTCTCGACGCAGGACTCGGCTTGCTGAAGCGCGCAC
GGCAAGAGGCGAGGGGCGGCGACTGGTGAGTACGCCAAAAATTTTGACTAGCGGAGGCTAGAAGGA
GAGAGATGGGTGCGAGAGCGTCAGTATTAAGCGGGGGAGAATTAGATCGCGATGGGAAAAAATTCG
GTTAAGGCCAGGGGGAAAGAAAAAATATAAATTAAAACATATAGTATGGGCAAGCAGGGAGCTAGAA
CGATTCGCAGTTAATACTGGCCTGTTAGAAACATCAGAAGGCTGTAGACAAATACTGGGACAGCTACA
ACCATCCCTTCAGACAGGATCAGAAGAACTTAGATCATTATATAATACAGTAGCAACCCTCTATTGTGT
GCATCAAAGGATAGAGATAAAAGACACCAAGGAAGCTTTAGACAAGATAGAGGAAGAGCAAAACAAA
AGTAAGAAAAAAGCACAGCAAGCAGCAGGATCTTCAGACCTGGAAATTCCCTACAATCCCCAAAGTCA
AGGAGTAGTAGAATCTATGAATAAAGAATTAAAGAAAATTATAGGACAGGTAAGAGATCAGGCTGAAC
ATCTTAAGACAGCAGTACAAATGGCAGTATTCATCCACAATTTTAAAAGAAAAGGGGGGATTGGGGG
GTACAGTGCAGGGGAAAGAATAGTAGACATAATAGCAACAGACATACAAACTAAAGAATTACAAAAAC
AAATTACAAAAATTCAAAATTTTCGGGTTTATTACAGGGACAGCAGAAATCCACTTTGGAAAGGACCA
GCAAAGCTCCTCTGGAAAGGTGAAGGGGCAGTAGTAATACAAGATAATAGTGACATAAAAGTAGTGC
CAAGAAGAAAAGCAAAGATCATTAGGGATTATGGAAAACAGATGGCAGGTGATGATTGTGTGGCAAG
TAGACAGGATGAGGATTAGAACATGGAAAAGTTTAGTAAAACACCATAAGGAGGAGATATGAGGGAC
AATTGGAGAAGTGAATTATATAAATATAAAGTAGTAAAAATTGAACCATTAGGAGTAGCACCCACCAA
GGCAAAGAGAAGAGTGGTGCAGAGAGAAAAAAGAGCAGTGGGAATAGGAGCTTTGTTCCTTGGGTT
CTTGGGAGCAGCAGGAAGCACTATGGGCGCAGCGTCAATGACGCTGACGGTACAGGCCAGACAATT
ATTGTCTGGTATAGTGCAGCAGCAGAACAATTTGCTGAGGGCTATTGAGGCGCAACAGCATCTGTTG
CAACTCACAGTCTGGGGCATCAAGCAGCTCCAGGCAAGAATCCTGGCTGTGGAAAGATACCTAAAGG
ATCAACAGCTCCTGGGGATTTGGGGTTGCTCTGGAAAACTCATTTGCACCACTGCTGTGCCTTGGAAT
GCTAGTTGGAGTAATAAATCTCTGGAACAGATTTGGAATCACACGACCTGGATGGAGTGGGACAGAG
AAATTAACAATTACACAAGCTTAATACACTCCTTAATTGAAGAATCGCAAAACCAGCAAGAAAAGAATG
AACAAGAATTATTGGAATTAGATAAATGGGCAAGTTTGTGGAATTGGTTTAACATAACAAATTGGCTGT
GGTATATAAAATTATTCATAATGATAGTAGGAGGCTTGGTAGGTTTAAGAATAGTTTTTGCTGTACTTT
CTATAGTGAATAGAGTTAGGCAGGGATATTCACCATTATCGTTTCAGACCCACCTCCCAACCCCGAGG
GGACCGAGCTCAAGCTTCGAACGCGTGGGGATCCTCTAGAGTCGAGCTCGCGAGGATCATCACCGG
TGCTAGCCGGAGCCAGAAGCACCATAAGGGACATGATAAGGGAGCCAGCAGACCTCTGATCTCTTCC
TGAATGCTAATCTTAAACATCCTGAGGAAGAATGGGACTTCCATTTGGGGTGGGCCTATGATAGGGTA
ATAAGACAGTAGTGAATATCAAGCTACAAAAAGCCCCCTTTCAAATTCTTCTCAGTCCTAACTTTTCAT
ACTAAGCCCAGTCCTTCCAAAGCAGACTGTGAAAGAGTGATAGTTCCGGGAGACTAGCACCGGCTAG
CCGAGCTTGGAACACTTTCCCTTCATTAAGAACCATCCTTGCTACTCAGCTGCAATCAATCCAGCCCCC
AGGTCTTCACTGAACCTTTTCCCATCTCTTCCAAAACATCTGTTTCTGAGAAGTCCTGTCCTATAGAGG
TCTTTCTTCCCACCGGATTTCTCCTACACCATTTACTCCCACTTGCAGAACTCCCGTGTACAAGTGTCT
TTACTGCTTTTATTTGCTCAACAAAATGCACATCTCATATAAAAATAAATGAGGAGCATGCACACACCA
CAAACACAAACAGGCATGCAGAAATACACATACACACTTCCCTCAATATAAACCCTTTGTGGCTCATAT
ATTTAAAAAGATGTAAAAAAAAGAGCTGAAGAAAATCATGTGTGATCTCTCAGCAGAATAGATTTATTA
TTTGTATTGCTTGCAGAATAAAGCCTATCCTTGAAAGCTCTGAATCATGGGCAAGAGGCTCAGTGGTA
TCTGGAGGACAGGGCACTGGCCACTGCAGTCACCATCTTCTGCCAGGAAGCCTGCACCTCAGGGGTG
AATTCTTTGCCAAAGTGAATGGCCAGCACGGTGACCAGCACGTTGCCCAGGAGCTGTGGGAGGAAGA
TAAGAGATATGAACATGATTAGCAAAAGGGCCTAGCTTGGACTCAGAATAATCCAGCCTTATCCCAAC
CATAAAATAAAAGCAGAATGGTAGCTGGATTGTAGCTGCTATTAGCAATATGAAACCTCTTACATCAGT
TACAATTTATATGCAGAAATATTTATATGCAGAAATATTGCTATTGCCTTAACCCAGAAATTATCACTGT
TATTCTTTAGAATGGTGCAAAGAGGCATGATACATTGTATCATTATTGCCCTGAAAGAAAGAGATTAG
GGAAAGTATTAGAAATAAGATAAACAAAAAAGTATATTAAAAGAAGAAAGCATTTTTTAAAATTACAAA
TGCAAAATTACCCTGATTTGGTCAATATGTGTACCCTGTTACTTCTCCCCTTCCTATGACATGAACTTA
ACCATAGAAAAGAAGGGGAAAGAAAACATCAAGGGTCCCATAGACTCACCTTGAAGTTCTCAGGATC
CACATGCAGCTTGTCACAGTGCAGTTCACTCAGCTGGGCAAAGGTGCCCTTGAGATCATCCAGGTGC
TTTATGGCATCTCCCAAGGAAGTCAGCACCTTCTTGCCATGTGCCTTGACTTTGGGGTTGCCCATGAT
GGCAGAGGCAGAGGACAGGTTGCCAAAGCTGTCAAAGAACCTCTGGGTCCATGGGTAGACAACCAG
GAGCCTGTGAGATTGACAAGAACAGTTTGACAGTCAGAAGGTGCCACAAATCCTGAGAAGCAACCTG
GACTTTTGCCAGGCACAGGGTCCTTCCTTCCCTCCCTTGTCCTGGTCACCAGAGCCTACCTTCCCAGG
GTTTCTCCTCCAGCATCTTCCACATTCACCTTGTCCCACAGGCTTGTGATAGTAGCCTTGTCCTCCTCT
GTGAAATGACCCATGGTGTCTGTTTGAGGTTGCTAGTGAACACAGTTGTGTCAGAAGCAAATGTAAGC
AATAGATGGCTCTGCCCTGACTTTTATGCCCAGCCCTGGCTCCTGCCCTCCCTGCTCCTGGGAGTAGA
TTGGCCAACCCTAGGGTGTGGCTCCACAGGGTGAGGTCTAAGTGATGACAGCCGTACCTGTCCTTGG
CTCTTCTGGCACTGGCTTAGGAGTTGGACTTCAAACCCTCAGCCCTCCCTCTAAGATATATCTCTTGG
CCCCATACCATCAGTACAAATTGCTACTAAAAACATCCTCCTTTGCAAGTGTATTTACGACGGTATCGA
TGTATGTGAGCATGTGTCCTCTAACAGCACAGGCCTTTTGCCACCTAGCTGTCCAGGGGTGCCTTAAA
ATGGCAAACAAGGTTTGTTTTCTTTTCCTGTTTTCATGCCTTCCTCTTCCATATCCTTGTTTCATATTAAT
ACATGTGTATAGATCCTAAAAATCTATACACATGTATTAATAAAGCCTGATTCTGCCGCTTCTAGGTAT
AGAGGCCACCTGCAAGATAAATATTTGATTCACAATAACTAATCATTCTATGGCAATTGATAACAACAA
ATATATATATATATATATATATACGTATATGTGTATATATATATATATATTCAGGAAATAATATATTCTAG
AATATGTCACATTCTGTCTCAGGCATCCATTTTCTTTATGATGCCGTTTGAGGTGGAGTTTTAGTCAGG
TGGTCAGCTTCTCCTTTTTTTTGCCATCTGCCCTGTAAGCATCCTGCTGGGGACCCAGATAGGAGTCA
TCACTCTAGGCTGAGAACATCTGGGCACACACCCTAAGCCTCAGCATGACTCATCATGACTCAGCATT
GCTGTGCTTGAGCCAGAAGGTTTGCTTAGAAGGTTACACAGAACCAGAAGGCGGGGGTGGGGCACT
GACCCCGACAGGGGCCTGGCCAGAACTGCTCATGCTTGGACTATGGGAGGTCACTAATGGAGACAC
ACAGAAATGTAACAGGAACTAAGGGAATTCCGGTGCCCTGCTTAGGAGCTTAATCTTTAATGAAAGCT
AAGCTTTCATTAAAAAAAGTCTAACCAGCTGCATTCGACTTTGACTGCAGCAGCTGGTTAGAAGGTTC
TACTGGAGGAGGGTCCCAGCCCATTGCTAAATTAACATCAGGCTCTGAGACTGGCAGTATATCTCTAA
CAGTGGTTGATGCTATCTTCTGGAACTTGCCTGCTACATTGAGACCACTGACCCATACATAGGAAGCC
CATAGCTCTGTCCTGAACTGTTAGGCCACTGGTCCAGAGAGTGTGCATCTCCTTTGATCCTCATAATA
ACCCTATGAGATAGACACAATTATTACTCTTACTTTATAGATGATGATCCTGAAAACATAGGAGTCAAG
GCACTTGCCCCTAGCTGGGGGTATAGGGGAGCAGTCCCATGTAGTAGTAGAATGAAAAATGCTGCTA
TGCTGTGCCTCCCCCACCTTTCCCATGTCTGCCCTCTACTCATGGTCTATCTCTCCTGGCTCCTGGGA
GTCATGGACTCCACCCAGCACCACCAACCTGACCTAACCACCTATCTGAGCCTGCCAGCCTATAACCC
ATCTGGGCCCTGATAGCTGGTGGCCAGCCCTGACCCCACCCCACCCTCCCTGGAACCTCTGATAGAC
ACATCTGGCACACCAGCTCGCAAAGTCACCGTGAGGGTCTTGTGTTTGCTGAGTCAAAATTCCTTGAA
ATCCAAGTCCTTAGAGACTCCTGCTCCCAAATTTACAGTCATAGACTTCTTCATGGCTGTCTCCTTTAT
CCACAGAATGATTCCTTTGCTTCATTGCCCCATCCATCTGATCCTCCTCATCAGTGCAGCACAGGGCC
CATGAGCAGTAGCTGCAGAGTCTCACATAGGTCTGGCACTGCCTCTGACATGTCCGACCTTAGGCAA
ATGCTTGACTCTTCTGAGCTCGGATCCCTTGAGCTCAGGAGGTCAAGGCTGCAGTGAGACATGATCTT
GCCACTGCACTCCAGCCTGGACAGCAGAGTGAAACCTTGCCTCACGAAACAGAATACAAAAACAAAC
AAACAAAAAACTGCTCCGCAATGCGCTTCCTTGATGCTCTACCACATAGGTCTGGGTACTTTGTACAC
ATTATCTCATTGCTGTTCATAATTGTTAGATTAATTTTGTAATATTGATATTATTCCTAGAAAGCTGAGG
CCTCAAGATGATAACTTTTATTTTCTGGACTTGTAATAGCTTTCTCTTGTATTCACCATGTTGTAACTTT
CTTAGAGTAGTAACAATATAAAGTTATTGTGAGTTTTTGCAAACACAGCAAACACAACGACCCATATAG
ACATTGATGTGAAATTGTCTATTGTCAATTTATGGGAAAACAAGTATGTACTTTTTCTACTAAGCCATT
GAAACAGGAATAACAGAACAAGATTGAAAGAATACATTTTCCGAAATTACTTGAGTATTATACAAAGAC
AAGCACGTGGACCTGGGAGGAGGGTTATTGTCCATGACTGGTGTGTGGAGACAAATGCAGGTTTATA
ATAGATGGGATGGCATCTAGCGCAATGACTTTGCCATCACTTTTAGAGAGCTCTTGGGGGCCCCAGTA
CACAAGAGGGGACGCAGGGTATATGTAGACATCTCATTCTTTTTCTTAGTGTGAGAATAAGAATAGCC
ATGACCTGAGTTTATAGACAATGAGCCCTTTTCTCTCTCCCACTCAGCAGCTATGAGATGGCTTGCCCT
GCCTCTCTACTAGGCTGACTCACTCCAAGGCCCAGCAATGGGCAGGGCTCTGTCAGGGCTTTGATAG
CACTATCTGCAGAGCCAGGGCCGAGAAGGGGTGGACTCCAGAGACTCTCCCTCCCATTCCCGAGCAG
GGTTTGCTTATTTATGCATTTAAATGATATATTTATTTTAAAAGAAATAACAGGAGACTGCCCAGCCCT
GGCTGTGACATGGAAACTATGTAGAATATTTTGGGTTCCATTTTTTTTTCCTTCTTTCAGTTAGAGGAA
AAGGGGCTCACTGCACATACACTAGACAGAAAGTCAGGAGCTTTGAATCCAAGCCTGATCATTTCCAT
GTCATACTGAGAAAGTCCCCACCCTTCTCTGAGCCTCAGTTTCTCTTTTTATAAGTAGGAGTCTGGAGT
AAATGATTTCCAATGGCTCTCATTTCAATACAAAATTTCCGTTTATTAAATGCATGAGCTTCCGTTACTC
CAAGACTGAGAAGGAAATTGAACCTGAGACTCATTGACTGGCAAGATGTCCCCAGAGGCTCTCATTCA
GCAATAAAATTCTCACCTTCACCCAGGCCCACTAGTGTCAGATTTGCATGCGTTCGCGTATCGACGTG
CAGTATTTAGCATGCCCCACCCATCTGCAAGGCATTCTGGATAGTGTCAAAACAGCCGGAAATCAAGT
CCGTTTATCTCAAACTTTAGCATTTTGGGAATAAATGATATTTGCTATGCTGGTTAAATTAGATTTTAGT
TAAATTTCCTGCTGAAGCTCTAGTACGATAAGTAACTTGACCTAAGTGTAAAGTTGAGATTTCCTTCAG
GTTTATATAGCTTGTGCGCCGCCTGGGTACCTCAGGATATGCCCTTGACTATTTGTCCGACATAGTCA
AGGGCATATCCTTTTTTGTGCGGCCGCATCGATGCCGTAGTACCTTTAAGACCAATGACTTACAAGGC
AGCTGTAGATCTTAGCCACTTTTTAAAAGAAAAGGGGGGACTGGAAGGGCTAATTCACTCCCAAAGAA
GACAAGATAGATCTGCTTTTTGCCTGTACTGGGTCTCTCTGGTTAGACCAGATCTGAGCCTGGGAGCT
CTCTGGCTAACTAGGGAACCCACTGCTTAAGCCTCAATAAAGCTTCAGCTGCTCGAGCTAGCAGATCT
TTTTCCCTCTGCCAAAAATTATGGGGACATCATGAAGCCCCTTGAGCATCTGACTTCTGGCTAATAAA
GGAAATTTATTTTCATTGCAATAGTGTGTTGGAATTTTTTGTGTCTCTCACTCGGAAGGACATATGGGA
GGGCAAATCATTTAAAACATCAGAATGAGTATTTGGTTTAGAGTTTGGCAACATATGCCCATATGCTG
GCTGCCATGAACAAAGGTTGGCTATAAAGAGGTCATCAGTATATGAAACAGCCCCCTGCTGTCCATTC
CTTATTCCATAGAAAAGCCTTGACTTGAGGTTAGATTTTTTTTATATTTTGTTTTGTGTTATTTTTTTCTT
TAACATCCCTAAAATTTTCCTTACATGTTTTACTAGCCAGATTTTTCCTCCTCTCCTGACTACTCCCAGT
CATAGCTGTCCCTCTTCTCTTATGGAGATCCCTCGACCTGCAGCCCAAGCTTGGCGTAATCATGGTCA
TAGCTGTTTCCTGTGTGAAATTGTTATCCGCTCACAATTCCACACAACATACGAGCCGGAAGCATAAA
GTGTAAAGCCTGGGGTGCCTAATGAGTGAGCTAACTCACATTAATTGCGTTGCGCTCACTGCCCGCTT
TCCAGTCGGGAAACCTGTCGTGCCAGCGGATCCGCATCTCAATTAGTCAGCAACCATAGTCCCGCCC
CTAACTCCGCCCATCCCGCCCCTAACTCCGCCCAGTTCCGCCCATTCTCCGCCCCATGGCTGACTAAT
TTTTTTTATTTATGCAGAGGCCGAGGCCGCCTCGGCCTCTGAGCTATTCCAGAAGTAGTGAGGAGGCT
TTTTTGGAGGCCTAGGCTTTTGCAAAAAGCTGTCGACTGCAGAGGCCTGCATGCAAGCTTGGCGTAA
TCATGGTCATAGCTGTTTCCTGTGTGAAATTGTTATCCGCTCACAATTCCACACAACATACGAGCCGG
AAGCATAAAGTGTAAAGCCTGGGGTGCCTAATGAGTGAGCTAACTCACATTAATTGCGTTGCGCTCAC
TGCCCGCTTTCCAGTCGGGAAACCTGTCGTGCCAGCTGCATTAATGAATCGGCCAACGCGCGGGGAG
AGGCGGTTTGCGTATTGGGCGCTCTTCCGCTTCCTCGCTCACTGACTCGCTGCGCTCGGTCGTTCGG
CTGCGGCGAGCGGTATCAGCTCACTCAAAGGCGGTAATACGGTTATCCACAGAATCAGGGGATAACG
CAGGAAAGAACATGTGAGCAAAAGGCCAGCAAAAGGCCAGGAACCGTAAAAAGGCCGCGTTGCTGG
CGTTTTTCCATAGGCTCCGCCCCCCTGACGAGCATCACAAAAATCGACGCTCAAGTCAGAGGTGGCG
AAACCCGACAGGACTATAAAGATACCAGGCGTTTCCCCCTGGAAGCTCCCTCGTGCGCTCTCCTGTTC
CGACCCTGCCGCTTACCGGATACCTGTCCGCCTTTCTCCCTTCGGGAAGCGTGGCGCTTTCTCATAGC
TCACGCTGTAGGTATCTCAGTTCGGTGTAGGTCGTTCGCTCCAAGCTGGGCTGTGTGCACGAACCCC
CCGTTCAGCCCGACCGCTGCGCCTTATCCGGTAACTATCGTCTTGAGTCCAACCCGGTAAGACACGA
CTTATCGCCACTGGCAGCAGCCACTGGTAACAGGATTAGCAGAGCGAGGTATGTAGGCGGTGCTACA
GAGTTCTTGAAGTGGTGGCCTAACTACGGCTACACTAGAAGAACAGTATTTGGTATCTGCGCTCTGCT
GAAGCCAGTTACCTTCGGAAAAAGAGTTGGTAGCTCTTGATCCGGCAAACAAACCACCGCTGGTAGC
GGTGGTTTTTTTGTTTGCAAGCAGCAGATTACGCGCAGAAAAAAAGGATCTCAAGAAGATCCTTTGAT
CTTTTCTACGGGGTCTGACGCTCAGTGGAACGAAAACTCACGTTAAGGGATTTTGGTCATGAGATTAT
CAAAAAGGATCTTCACCTAGATCCTTTTAAATTAAAAATGAAGTTTTAAATCAATCTAAAGTATATATGA
GTAAACTTGGTCTGACAGTTACCAATGCTTAATCAGTGAGGCACCTATCTCAGCGATCTGTCTATTTCG
TTCATCCATAGTTGCCTGACTCCCCGTCGTGTAGATAACTACGATACGGGAGGGCTTACCATCTGGCC
CCAGTGCTGCAATGATACCGCGAGACCCACGCTCACCGGCTCCAGATTTATCAGCAATAAACCAGCC
AGCCGGAAGGGCCGAGCGCAGAAGTGGTCCTGCAACTTTATCCGCCTCCATCCAGTCTATTAATTGTT
GCCGGGAAGCTAGAGTAAGTAGTTCGCCAGTTAATAGTTTGCGCAACGTTGTTGCCATTGCTACAGG
CATCGTGGTGTCACGCTCGTCGTTTGGTATGGCTTCATTCAGCTCCGGTTCCCAACGATCAAGGCGAG
TTACATGATCCCCCATGTTGTGCAAAAAAGCGGTTAGCTCCTTCGGTCCTCCGATCGTTGTCAGAAGT
AAGTTGGCCGCAGTGTTATCACTCATGGTTATGGCAGCACTGCATAATTCTCTTACTGTCATGCCATCC
GTAAGATGCTTTTCTGTGACTGGTGAGTACTCAACCAAGTCATTCTGAGAATAGTGTATGCGGCGACC
GAGTTGCTCTTGCCCGGCGTCAATACGGGATAATACCGCGCCACATAGCAGAACTTTAAAAGTGCTCA
TCATTGGAAAACGTTCTTCGGGGCGAAAACTCTCAAGGATCTTACCGCTGTTGAGATCCAGTTCGATG
TAACCCACTCGTGCACCCAACTGATCTTCAGCATCTTTTACTTTCACCAGCGTTTCTGGGTGAGCAAAA
ACAGGAAGGCAAAATGCCGCAAAAAAGGGAATAAGGGCGACACGGAAATGTTGAATACTCATACTCT
TCCTTTTTCAATATTATTGAAGCATTTATCAGGGTTATTGTCTCATGAGCGGATACATATTTGAATGTAT
TTAGAAAAATAAACAAATAGGGGTTCCGCGCACATTTCCCCGAAAAGTGCCACCTGACGTCTAAGAAA
CCATTATTATCATGACATTAACCTATAAAAATAGGCGTATCACGAGGCCCTTTCGTCTCGCGCGTTTCG
GTGATGACGGTGAAAACCTCTGACACATGCAGCTCCCGGAGACGGTCACAGCTTGTCTGTAAGCGGA
TGCCGGGAGCAGACAAGCCCGTCAGGGCGCGTCAGCGGGTGTTGGCGGGTGTCGGGGCTGGCTTA
ACTATGCGGCATCAGAGCAGATTGTACTGAGAGTGCACCATATGCGGTGTGAAATACCGCACAGATG
CGTAAGGAGAAAATACCGCATCAGGCGCCATTCGCCATTCAGGCTGCGCAACTGTTGGGAAGGGCGA
TCGGTGCGGGCCTCTTCGCTATTACGCCAGCTGGCGAAAGGGGGATGTGCTGCAAGGCGATTAAGTT
GGGTAACGCCAGGGTTTTCCCAGTCACGACGTTGTAAAACGACGGCCAGTGAATTCGGCCGCCTCGG
CCAAACAGCCCTTGAGTTTACCACTCCCTATCAGTGATAGAGAAAAGTGAAAGTCGAGTTTACCACTC
CCTATCAGTGATAGAGAAAAGTGAAAGTCGAGTTTACCACTCCCTATCAGTGATAGAGAAAAGTGAAA
GTCGAGTTTACCACTCCCTATCAGTGATAGAGAAAAGTGAAAGTCGAGTTATTTCCCCGAAAAGTGCC
ACCTGACGTCTAAGAAACCATTATTATCATGACATTACCTATAAAAATAGGCGTATCACGAGGCCCTTT
CGTCTCGCGCGTTTCGGTGATGACGGTGAAAACCTCTGACACATGCAGCTCCCGGAGACGGTCACAG
CTTGTCTGTAAGCGGATGCCGGGAGCAGACAAGCCCGTCAGGGCGCGTCAGCGGGTGTTGGCGGGT
GTCGGGGCTGGCTTAACTATGCGGCATCAGAGCAGATTGTACTGAGAGTGCACCATATGCGGTGTGA
AATACCGCACAGATGCGTAAGGAGAAAATACCGCATCAGGCGCCATTCGCCATTCAGGCTGCGCAAC
TGTTGGGAAGGGCGATCGGTGCGGGCCTCTTCGCTATTACGCCAGCTGGCGAAAGGGGGATGTGCT
GCAAGGCGATTAAGTTGGGTAACGCCAGGGTTTTCCCAGTCACGACGTTGTAAAACGACGGCCAGTG
AATTC
pCalH32 TL20c rGbGM 7SKsh734 400 2AT (SEQ ID NO: 110)
GGCCGCCTCGGCCAAACAGCCCTTGAGTTTACCACTCCCTATCAGTGATAGAGAAAAGTGAAAGTCG
AGTTTACCACTCCCTATCAGTGATAGAGAAAAGTGAAAGTCGAGTTTACCACTCCCTATCAGTGATAG
AGAAAAGTGAAAGTCGAGTTTACCACTCCCTATCAGTGATAGAGAAAAGTGAAAGTCGAGTTTACCAG
TCCCTATCAGTGATAGAGAAAAGTGAAAGTCGAGTTTACCACTCCCTATCAGTGATAGAGAAAAGTGA
AAGTCGAGTTTACCACTCCCTATCAGTGATAGAGAAAAGTGAAAGTCGAGCTCGCCATGGGAGGCGT
GGCCTGGGCGGGACTGGGGAGTGGCGAGCCCTCAGATCCTGCATATAAGCAGCTGCTTTTTGCCTGT
ACTGGGTCTCTCTGGTTAGACCAGATCTGAGCCTGGGAGCTCTCTGGCTAACTAGGGAACCCACTGC
TTAAGCCTCAATAAAGCTTGCCTTGAGTGCTTCAAGTAGTGTGTGCCCGTCTGTTGTGTGACTCTGGT
AACTAGAGATCCCTCAGACCCTTTTAGTCAGTGTGGAAAATCTCTAGCAGTGGCGCCCGAACAGGGA
CTTGAAAGCGAAAGGGAAACCAGAGGAGCTCTCTCGACGCAGGACTCGGCTTGCTGAAGCGCGCAC
GGCAAGAGGCGAGGGGCGGCGACTGGTGAGTACGCCAAAAATTTTGACTAGCGGAGGCTAGAAGGA
GAGAGATGGGTGCGAGAGCGTCAGTATTAAGCGGGGGAGAATTAGATCGCGATGGGAAAAAATTCG
GTTAAGGCCAGGGGGAAAGAAAAAATATAAATTAAAACATATAGTATGGGCAAGCAGGGAGCTAGAA
CGATTCGCAGTTAATACTGGCCTGTTAGAAACATCAGAAGGCTGTAGACAAATACTGGGACAGCTACA
ACCATCCCTTCAGACAGGATCAGAAGAACTTAGATCATTATATAATACAGTAGCAACCCTCTATTGTGT
GCATCAAAGGATAGAGATAAAAGACACCAAGGAAGCTTTAGACAAGATAGAGGAAGAGCAAAACAAA
AGTAAGAAAAAAGCACAGCAAGCAGCAGGATCTTCAGACCTGGAAATTCCCTACAATCCCCAAAGTCA
AGGAGTAGTAGAATCTATGAATAAAGAATTAAAGAAAATTATAGGACAGGTAAGAGATCAGGCTGAAC
ATCTTAAGACAGCAGTACAAATGGCAGTATTCATCCACAATTTTAAAAGAAAAGGGGGGATTGGGGG
GTACAGTGCAGGGGAAAGAATAGTAGACATAATAGCAACAGACATACAAACTAAAGAATTACAAAAAC
AAATTACAAAAATTCAAAATTTTCGGGTTTATTACAGGGACAGCAGAAATCCACTTTGGAAAGGACCA
GCAAAGCTCCTCTGGAAAGGTGAAGGGGCAGTAGTAATACAAGATAATAGTGACATAAAAGTAGTGC
CAAGAAGAAAAGCAAAGATCATTAGGGATTATGGAAAACAGATGGCAGGTGATGATTGTGTGGCAAG
TAGACAGGATGAGGATTAGAACATGGAAAAGTTTAGTAAAACACCATAAGGAGGAGATATGAGGGAC
AATTGGAGAAGTGAATTATATAAATATAAAGTAGTAAAAATTGAACCATTAGGAGTAGCACCCACCAA
GGCAAAGAGAAGAGTGGTGCAGAGAGAAAAAAGAGCAGTGGGAATAGGAGCTTTGTTCCTTGGGTT
CTTGGGAGCAGCAGGAAGCACTATGGGCGCAGCGTCAATGACGCTGACGGTACAGGCCAGACAATT
ATTGTCTGGTATAGTGCAGCAGCAGAACAATTTGCTGAGGGCTATTGAGGCGCAACAGCATCTGTTG
CAACTCACAGTCTGGGGCATCAAGCAGCTCCAGGCAAGAATCCTGGCTGTGGAAAGATACCTAAAGG
ATCAACAGCTCCTGGGGATTTGGGGTTGCTCTGGAAAACTCATTTGCACCACTGCTGTGCCTTGGAAT
GCTAGTTGGAGTAATAAATCTCTGGAACAGATTTGGAATCACACGACCTGGATGGAGTGGGACAGAG
AAATTAACAATTACACAAGCTTAATACACTCCTTAATTGAAGAATCGCAAAACCAGCAAGAAAAGAATG
AACAAGAATTATTGGAATTAGATAAATGGGCAAGTTTGTGGAATTGGTTTAACATAACAAATTGGCTGT
GGTATATAAAATTATTCATAATGATAGTAGGAGGCTTGGTAGGTTTAAGAATAGTTTTTGCTGTACTTT
CTATAGTGAATAGAGTTAGGCAGGGATATTCACCATTATCGTTTCAGACCCACCTCCCAACCCCGAGG
GGACCGAGCTCAAGCTTCGAACGCGTGGGGATCCTCTAGAGTCGAGCTCGCGAGGATCATCACCGG
TGCTAGCCGGAGCCAGAAGCACCATAAGGGACATGATAAGGGAGCCAGCAGACCTCTGATCTCTTCC
TGAATGCTAATCTTAAACATCCTGAGGAAGAATGGGACTTCCATTTGGGGTGGGCCTATGATAGGGTA
ATAAGACAGTAGTGAATATCAAGCTACAAAAAGCCCCCTTTCAAATTCTTCTCAGTCCTAACTTTTCAT
ACTAAGCCCAGTCCTTCCAAAGCAGACTGTGAAAGAGTGATAGTTCCGGGAGACTAGCACCGGCTAG
CCGAGCTTGGAACACTTTCCCTTCATTAAGAACCATCCTTGCTACTCAGCTGCAATCAATCCAGCCCCC
AGGTCTTCACTGAACCTTTTCCCATCTCTTCCAAAACATCTGTTTCTGAGAAGTCCTGTCCTATAGAGG
TCTTTCTTCCCACCGGATTTCTCCTACACCATTTACTCCCACTTGCAGAACTCCCGTGTACAAGTGTCT
TTACTGCTTTTATTTGCTCAACAAAATGCACATCTCATATAAAAATAAATGAGGAGCATGCACACACCA
CAAACACAAACAGGCATGCAGAAATACACATACACACTTCCCTCAATATAAACCCTTTGTGGCTCATAT
ATTTAAAAAGATGTAAAAAAAAGAGCTGAAGAAAATCATGTGTGATCTCTCAGCAGAATAGATTTATTA
TTTGTATTGCTTGCAGAATAAAGCCTATCCTTGAAAGCTCTGAATCATGGGCAAGAGGCTCAGTGGTA
TCTGGAGGACAGGGCACTGGCCACTGCAGTCACCATCTTCTGCCAGGAAGCCTGCACCTCAGGGGTG
AATTCTTTGCCAAAGTGAATGGCCAGCACGGTGACCAGCACGTTGCCCAGGAGCTGTGGGAGGAAGA
TAAGAGGTATGAACATGATTAGCAAAAGGGCCTAGCTTGGACTCAGAATAATCCAGCCTTATCCCAAC
CATAAAATAAAAGCAGAATGGTAGCTGGATTGTAGCTGCTATTAGCAATATGAAACCTCTTACATCAGT
TACAATTTATATGCAGAAATATTTATATGCAGAAATATTGCTATTGCCTTAACCCAGAAATTATCACTGT
TATTCTTTAGAATGGTGCAAAGAGGCATGATACATTGTATCATTATTGCCCTGAAAGAAAGAGATTAG
GGAAAGTATTAGAAATAAGATAAACAAAAAAGTATATTAAAAGAAGAAAGCATTTTTTAAAATTACAAA
TGCAAAATTACCCTGATTTGGTCAATATGTGTACCCTGTTACTTCTCCCCTTCCTATGACATGAACTTA
ACCATAGAAAAGAAGGGGAAAGAAAACATCAAGGGTCCCATAGACTCACCTTGAAGTTCTCAGGATC
CACATGCAGCTTGTCACAGTGCAGTTCACTCAGCTGGGCAAAGGTGCCCTTGAGATCATCCAGGTGC
TTTATGGCATCTCCCAAGGAAGTCAGCACCTTCTTGCCATGTGCCTTGACTTTGGGGTTGCCCATGAT
GGCAGAGGCAGAGGACAGGTTGCCAAAGCTGTCAAAGAACCTCTGGGTCCATGGGTAGACAACCAG
GAGCCTGTGAGATTGACAAGAACAGTTTGACAGTCAGAAGGTGCCACAAATCCTGAGAAGCAACCTG
GACTTTTGCCAGGCACAGGGTCCTTCCTTCCCTCCCTTGTCCTGGTCACCAGAGCCTACCTTCCCAGG
GTTTCTCCTCCAGCATCTTCCACATTCACCTTGTCCCACAGGCTTGTGATAGTAGCCTTGTCCTCCTCT
GTGAAATGACCCATGGTGTCTGTTTGAGGTTGCTAGTGAACACAGTTGTGTCAGAAGCAAATGTAAGC
AATAGATGGCTCTGCCCTGACTTTTATGCCCAGCCCTGGCTCCTGCCCTCCCTGCTCCTGGGAGTAGA
TTGGCCAACCCTAGGGTGTGGCTCCACAGGGTGAGGTCTAAGTGATGACAGCCGTACCTGTCCTTGG
CTCTTCTGGCACTGGCTTAGGAGTTGGACTTCAAACCCTCAGCCCTCCCTCTAAGATATATCTCTTGG
CCCCATACCATCAGTACAAATTGCTACTAAAAACATCCTCCTTTGCAAGTGTATTTACGACGGTATCGA
TGTATGTGAGCATGTGTCCTCTAACAGCACAGGCCTTTTGCCACCTAGCTGTCCAGGGGTGCCTTAAA
ATGGCAAACAAGGTTTGTTTTCTTTTCCTGTTTTCATGCCTTCCTCTTCCATATCCTTGTTTCATATTAAT
ACATGTGTATAGATCCTAAAAATCTATACACATGTATTAATAAAGCCTGATTCTGCCGCTTCTAGGTAT
AGAGGCCACCTGCAAGATAAATATTTGATTCACAATAACTAATCATTCTATGGCAATTGATAACAACAA
ATATATATATATATATATATATACGTATATGTGTATATATATATATATATTCAGGAAATAATATATTCTAG
AATATGTCACATTCTGTCTCAGGCATCCATTTTCTTTATGATGCCGTTTGAGGTGGAGTTTTAGTCAGG
TGGTCAGCTTCTCCTTTTTTTTGCCATCTGCCCTGTAAGCATCCTGCTGGGGACCCAGATAGGAGTCA
TCACTCTAGGCTGAGAACATCTGGGCACACACCCTAAGCCTCAGCATGACTCATCATGACTCAGCATT
GCTGTGCTTGAGCCAGAAGGTTTGCTTAGAAGGTTACACAGAACCAGAAGGCGGGGGTGGGGCACT
GACCCCGACAGGGGCCTGGCCAGAACTGCTCATGCTTGGACTATGGGAGGTCACTAATGGAGACAC
ACAGAAATGTAACAGGAACTAAGGGAATTCCGGTGCCCTGCTTAGGAGCTTAATCTTTAATGAAAGCT
AAGCTTTCATTAAAAAAAGTCTAACCAGCTGCATTCGACTTTGACTGCAGCAGCTGGTTAGAAGGTTC
TACTGGAGGAGGGTCCCAGCCCATTGCTAAATTAACATCAGGCTCTGAGACTGGCAGTATATCTCTAA
CAGTGGTTGATGCTATCTTCTGGAACTTGCCTGCTACATTGAGACCACTGACCCATACATAGGAAGCC
CATAGCTCTGTCCTGAACTGTTAGGCCACTGGTCCAGAGAGTGTGCATCTCCTTTGATCCTCATAATA
ACCCTATGAGATAGACACAATTATTACTCTTACTTTATAGATGATGATCCTGAAAACATAGGAGTCAAG
GCACTTGCCCCTAGCTGGGGGTATAGGGGAGCAGTCCCATGTAGTAGTAGAATGAAAAATGCTGCTA
TGCTGTGCCTCCCCCACCTTTCCCATGTCTGCCCTCTACTCATGGTCTATCTCTCCTGGCTCCTGGGA
GTCATGGACTCCACCCAGCACCACCAACCTGACCTAACCACCTATCTGAGCCTGCCAGCCTATAACCC
ATCTGGGCCCTGATAGCTGGTGGCCAGCCCTGACCCCACCCCACCCTCCCTGGAACCTCTGATAGAC
ACATCTGGCACACCAGCTCGCAAAGTCACCGTGAGGGTCTTGTGTTTGCTGAGTCAAAATTCCTTGAA
ATCCAAGTCCTTAGAGACTCCTGCTCCCAAATTTACAGTCATAGACTTCTTCATGGCTGTCTCCTTTAT
CCACAGAATGATTCCTTTGCTTCATTGCCCCATCCATCTGATCCTCCTCATCAGTGCAGCACAGGGCC
CATGAGCAGTAGCTGCAGAGTCTCACATAGGTCTGGCACTGCCTCTGACATGTCCGACCTTAGGCAA
ATGCTTGACTCTTCTGAGCTCGGATCCCTTGAGCTCAGGAGGTCAAGGCTGCAGTGAGACATGATCTT
GCCACTGCACTCCAGCCTGGACAGCAGAGTGAAACCTTGCCTCACGAAACAGAATACAAAAACAAAC
AAACAAAAAACTGCTCCGCAATGCGCTTCCTTGATGCTCTACCACATAGGTCTGGGTACTTTGTACAC
ATTATCTCATTGCTGTTCATAATTGTTAGATTAATTTTGTAATATTGATATTATTCCTAGAAAGCTGAGG
CCTCAAGATGATAACTTTTATTTTCTGGACTTGTAATAGCTTTCTCTTGTATTCACCATGTTGTAACTTT
CTTAGAGTAGTAACAATATAAAGTTATTGTGAGTTTTTGCAAACACAGCAAACACAACGACCCATATAG
ACATTGATGTGAAATTGTCTATTGTCAATTTATGGGAAAACAAGTATGTACTTTTTCTACTAAGCCATT
GAAACAGGAATAACAGAACAAGATTGAAAGAATACATTTTCCGAAATTACTTGAGTATTATACAAAGAC
AAGCACGTGGACCTGGGAGGAGGGTTATTGTCCATGACTGGTGTGTGGAGACAAATGCAGGTTTATA
ATAGATGGGATGGCATCTAGCGCAATGACTTTGCCATCACTTTTAGAGAGCTCTTGGGGGCCCCAGTA
CACAAGAGGGGACGCAGGGTATATGTAGACATCTCATTCTTTTTCTTAGTGTGAGAATAAGAATAGCC
ATGACCTGAGTTTATAGACAATGAGCCCTTTTCTCTCTCCCACTCAGCAGCTATGAGATGGCTTGCCCT
GCCTCTCTACTAGGCTGACTCACTCCAAGGCCCAGCAATGGGCAGGGCTCTGTCAGGGCTTTGATAG
CACTATCTGCAGAGCCAGGGCCGAGAAGGGGTGGACTCCAGAGACTCTCCCTCCCATTCCCGAGCAG
GGTTTGCTTATTTATGCATTTAAATGATATATTTATTTTAAAAGAAATAACAGGAGACTGCCCAGCCCT
GGCTGTGACATGGAAACTATGTAGAATATTTTGGGTTCCATTTTTTTTTCCTTCTTTCAGTTAGAGGAA
AAGGGGCTCACTGCACATACACTAGACAGAAAGTCAGGAGCTTTGAATCCAAGCCTGATCATTTCCAT
GTCATACTGAGAAAGTCCCCACCCTTCTCTGAGCCTCAGTTTCTCTTTTTATAAGTAGGAGTCTGGAGT
AAATGATTTCCAATGGCTCTCATTTCAATACAAAATTTCCGTTTATTAAATGCATGAGCTTCCGTTACTC
CAAGACTGAGAAGGAAATTGAACCTGAGACTCATTGACTGGCAAGATGTCCCCAGAGGCTCTCATTCA
GCAATAAAATTCTCACCTTCACCCAGGCCCACTAGTGTCAGATTTGCATGCGTTCGCGTATCGACGTG
CAGTATTTAGCATGCCCCACCCATCTGCAAGGCATTCTGGATAGTGTCAAAACAGCCGGAAATCAAGT
CCGTTTATCTCAAACTTTAGCATTTTGGGAATAAATGATATTTGCTATGCTGGTTAAATTAGATTTTAGT
TAAATTTCCTGCTGAAGCTCTAGTACGATAAGTAACTTGACCTAAGTGTAAAGTTGAGATTTCCTTCAG
GTTTATATAGCTTGTGCGCCGCCTGGGTACCTCAGGATATGCCCTTGACTATTTGTCCGACATAGTCA
AGGGCATATCCTTTTTTGTGCGGCCGCATCGATGCCGTAGTACCTTTAAGACCAATGACTTACAAGGC
AGCTGTAGATCTTAGCCACTTTTTAAAAGAAAAGGGGGGACTGGAAGGGCTAATTCACTCCCAAAGAA
GACAAGATCCCTGCAGGCATTCAAGGCCAGGCTGGATGTGGCTCTGGGCAGCCTGGGCTGCTGGTT
GATGACCCTGCACATAGCAGGGGGTTGGATCTGGATGAGCACTGTGCTCCTTTGCAACCCAGGCCGT
TCTATGATTCTGTCATTCTAAATCTCTCTTTCAGCCTAAAGCTTTTTCCCCGTATCCCCCCIGGTGTCTG
CIGGCTCAAAGAGCAGCGAGAAGCGTTCAGAGGAAAGCGATCCCGTGCCACCTTCCCCGTGCCCGG
GCTGTCCCCGCACGCTGCCGGCTCGGGGATGCGGGGGGAGCGCCGGACCGGAGCGGAGCCCCGG
GCGGCTCGCTGCTGCCCCCTAGCGGGGGAGGGACGTAATTACATCCCTGGGGGCTTTGGGGGGGG
GCTGTCCCCGTGAGCTCCCCAGATCTGCTTTTTGCCTGTACTGGGTCTCTCTGGTTAGACCAGATCTG
AGCCTGGGAGCTCTCTGGCTAACTAGGGAACCCACTGCTTAAGCCTCAATAAAGCTTCAGCTGCTCGA
GCTAGCAGATCTTTTTCCCTCTGCCAAAAATTATGGGGACATCATGAAGCCCCTTGAGCATCTGACTT
CTGGCTAATAAAGGAAATTTATTTTCATTGCAATAGTGTGTTGGAATTTTTTGTGTCTCTCACTCGGAA
GGACATATGGGAGGGCAAATCATTTAAAACATCAGAATGAGTATTTGGTTTAGAGTTTGGCAACATAT
GCCCATATGCTGGCTGCCATGAACAAAGGTTGGCTATAAAGAGGTCATCAGTATATGAAACAGCCCC
CTGCTGTCCATTCCTTATTCCATAGAAAAGCCTTGACTTGAGGTTAGATTTTTTTTATATTTTGTTTTGT
GTTATTTTTTTCTTTAACATCCCTAAAATTTTCCTTACATGTTTTACTAGCCAGATTTTTCCTCCTCTCCT
GACTACTCCCAGTCATAGCTGTCCCTCTTCTCTTATGGAGATCCCTCGACCTGCAGCCCAAGCTTGGC
GTAATCATGGTCATAGCTGTTTCCTGTGTGAAATTGTTATCCGCTCACAATTCCACACAACATACGAGC
CGGAAGCATAAAGTGTAAAGCCTGGGGTGCCTAATGAGTGAGCTAACTCACATTAATTGCGTTGCGC
TCACTGCCCGCTTTCCAGTCGGGAAACCTGTCGTGCCAGCGGATCCGCATCTCAATTAGTCAGCAACC
ATAGTCCCGCCCCTAACTCCGCCCATCCCGCCCCTAACTCCGCCCAGTTCCGCCCATTCTCCGCCCCA
TGGCTGACTAATTTTTTTTATTTATGCAGAGGCCGAGGCCGCCTCGGCCTCTGAGCTATTCCAGAAGT
AGTGAGGAGGCTTTTTTGGAGGCCTAGGCTTTTGCAAAAAGCTGTCGACTGCAGAGGCCTGCATGCA
AGCTTGGCGTAATCATGGTCATAGCTGTTTCCTGTGTGAAATTGTTATCCGCTCACAATTCCACACAAC
ATACGAGCCGGAAGCATAAAGTGTAAAGCCTGGGGTGCCTAATGAGTGAGCTAACTCACATTAATTG
CGTTGCGCTCACTGCCCGCTTTCCAGTCGGGAAACCTGTCGTGCCAGCTGCATTAATGAATCGGCCA
ACGCGCGGGGAGAGGCGGTTTGCGTATTGGGCGCTCTTCCGCTTCCTCGCTCACTGACTCGCTGCGC
TCGGTCGTTCGGCTGCGGCGAGCGGTATCAGCTCACTCAAAGGCGGTAATACGGTTATCCACAGAAT
CAGGGGATAACGCAGGAAAGAACATGTGAGCAAAAGGCCAGCAAAAGGCCAGGAACCGTAAAAAGG
CCGCGTTGCTGGCGTTTTTCCATAGGCTCCGCCCCCCTGACGAGCATCACAAAAATCGACGCTCAAGT
CAGAGGTGGCGAAACCCGACAGGACTATAAAGATACCAGGCGTTTCCCCCTGGAAGCTCCCTCGTGC
GCTCTCCTGTTCCGACCCTGCCGCTTACCGGATACCTGTCCGCCTTTCTCCCTTCGGGAAGCGTGGCG
CTTTCTCATAGCTCACGCTGTAGGTATCTCAGTTCGGTGTAGGTCGTTCGCTCCAAGCTGGGCTGTGT
GCACGAACCCCCCGTTCAGCCCGACCGCTGCGCCTTATCCGGTAACTATCGTCTTGAGTCCAACCCG
GTAAGACACGACTTATCGCCACTGGCAGCAGCCACTGGTAACAGGATTAGCAGAGCGAGGTATGTAG
GCGGTGCTACAGAGTTCTTGAAGTGGTGGCCTAACTACGGCTACACTAGAAGAACAGTATTTGGTATC
TGCGCTCTGCTGAAGCCAGTTACCTTCGGAAAAAGAGTTGGTAGCTCTTGATCCGGCAAACAAACCAC
CGCTGGTAGCGGTGGTTTTTTTGTTTGCAAGCAGCAGATTACGCGCAGAAAAAAAGGATCTCAAGAA
GATCCTTTGATCTTTTCTACGGGGTCTGACGCTCAGTGGAACGAAAACTCACGTTAAGGGATTTTGGT
CATGAGATTATCAAAAAGGATCTTCACCTAGATCCTTTTAAATTAAAAATGAAGTTTTAAATCAATCTAA
AGTATATATGAGTAAACTTGGTCTGACAGTTACCAATGCTTAATCAGTGAGGCACCTATCTCAGCGAT
CTGTCTATTTCGTTCATCCATAGTTGCCTGACTCCCCGTCGTGTAGATAACTACGATACGGGAGGGCT
TACCATCTGGCCCCAGTGCTGCAATGATACCGCGAGACCCACGCTCACCGGCTCCAGATTTATCAGC
AATAAACCAGCCAGCCGGAAGGGCCGAGCGCAGAAGTGGTCCTGCAACTTTATCCGCCTCCATCCAG
TCTATTAATTGTTGCCGGGAAGCTAGAGTAAGTAGTTCGCCAGTTAATAGTTTGCGCAACGTTGTTGC
CATTGCTACAGGCATCGTGGTGTCACGCTCGTCGTTTGGTATGGCTTCATTCAGCTCCGGTTCCCAAC
GATCAAGGCGAGTTACATGATCCCCCATGTTGTGCAAAAAAGCGGTTAGCTCCTTCGGTCCTCCGATC
GTTGTCAGAAGTAAGTTGGCCGCAGTGTTATCACTCATGGTTATGGCAGCACTGCATAATTCTCTTAC
TGTCATGCCATCCGTAAGATGCTTTTCTGTGACTGGTGAGTACTCAACCAAGTCATTCTGAGAATAGT
GTATGCGGCGACCGAGTTGCTCTTGCCCGGCGTCAATACGGGATAATACCGCGCCACATAGCAGAAC
TTTAAAAGTGCTCATCATTGGAAAACGTTCTTCGGGGCGAAAACTCTCAAGGATCTTACCGCTGTTGA
GATCCAGTTCGATGTAACCCACTCGTGCACCCAACTGATCTTCAGCATCTTTTACTTTCACCAGCGTTT
CTGGGTGAGCAAAAACAGGAAGGCAAAATGCCGCAAAAAAGGGAATAAGGGCGACACGGAAATGTT
GAATACTCATACTCTTCCTTTTTCAATATTATTGAAGCATTTATCAGGGTTATTGTCTCATGAGCGGATA
CATATTTGAATGTATTTAGAAAAATAAACAAATAGGGGTTCCGCGCACATTTCCCCGAAAAGTGCCAC
CTGACGTCTAAGAAACCATTATTATCATGACATTAACCTATAAAAATAGGCGTATCACGAGGCCCTTTC
GTCTCGCGCGTTTCGGTGATGACGGTGAAAACCTCTGACACATGCAGCTCCCGGAGACGGTCACAGC
TTGTCTGTAAGCGGATGCCGGGAGCAGACAAGCCCGTCAGGGCGCGTCAGCGGGTGTTGGCGGGTG
TCGGGGCTGGCTTAACTATGCGGCATCAGAGCAGATTGTACTGAGAGTGCACCATATGCGGTGTGAA
ATACCGCACAGATGCGTAAGGAGAAAATACCGCATCAGGCGCCATTCGCCATTCAGGCTGCGCAACT
GTTGGGAAGGGCGATCGGTGCGGGCCTCTTCGCTATTACGCCAGCTGGCGAAAGGGGGATGTGCTG
CAAGGCGATTAAGTTGGGTAACGCCAGGGTTTTCCCAGTCACGACGTTGTAAAACGACGGCCAGTGA
ATTC
pCalH13 TL20c rGbGM G3320A 7SKsh734 400 2AT (SEQ ID NO: 111)
GGCCGCCTCGGCCAAACAGCCCTTGAGTTTACCACTCCCTATCAGTGATAGAGAAAAGTGAAAGTCG
AGTTTACCACTCCCTATCAGTGATAGAGAAAAGTGAAAGTCGAGTTTACCACTCCCTATCAGTGATAG
AGAAAAGTGAAAGTCGAGTTTACCACTCCCTATCAGTGATAGAGAAAAGTGAAAGTCGAGTTTACCAG
TCCCTATCAGTGATAGAGAAAAGTGAAAGTCGAGTTTACCACTCCCTATCAGTGATAGAGAAAAGTGA
AAGTCGAGTTTACCACTCCCTATCAGTGATAGAGAAAAGTGAAAGTCGAGCTCGCCATGGGAGGCGT
GGCCTGGGCGGGACTGGGGAGTGGCGAGCCCTCAGATCCTGCATATAAGCAGCTGCTTTTTGCCTGT
ACTGGGTCTCTCTGGTTAGACCAGATCTGAGCCTGGGAGCTCTCTGGCTAACTAGGGAACCCACTGC
TTAAGCCTCAATAAAGCTTGCCTTGAGTGCTTCAAGTAGTGTGTGCCCGTCTGTTGTGTGACTCTGGT
AACTAGAGATCCCTCAGACCCTTTTAGTCAGTGTGGAAAATCTCTAGCAGTGGCGCCCGAACAGGGA
CTTGAAAGCGAAAGGGAAACCAGAGGAGCTCTCTCGACGCAGGACTCGGCTTGCTGAAGCGCGCAC
GGCAAGAGGCGAGGGGCGGCGACTGGTGAGTACGCCAAAAATTTTGACTAGCGGAGGCTAGAAGGA
GAGAGATGGGTGCGAGAGCGTCAGTATTAAGCGGGGGAGAATTAGATCGCGATGGGAAAAAATTCG
GTTAAGGCCAGGGGGAAAGAAAAAATATAAATTAAAACATATAGTATGGGCAAGCAGGGAGCTAGAA
CGATTCGCAGTTAATACTGGCCTGTTAGAAACATCAGAAGGCTGTAGACAAATACTGGGACAGCTACA
ACCATCCCTTCAGACAGGATCAGAAGAACTTAGATCATTATATAATACAGTAGCAACCCTCTATTGTGT
GCATCAAAGGATAGAGATAAAAGACACCAAGGAAGCTTTAGACAAGATAGAGGAAGAGCAAAACAAA
AGTAAGAAAAAAGCACAGCAAGCAGCAGGATCTTCAGACCTGGAAATTCCCTACAATCCCCAAAGTCA
AGGAGTAGTAGAATCTATGAATAAAGAATTAAAGAAAATTATAGGACAGGTAAGAGATCAGGCTGAAC
ATCTTAAGACAGCAGTACAAATGGCAGTATTCATCCACAATTTTAAAAGAAAAGGGGGGATTGGGGG
GTACAGTGCAGGGGAAAGAATAGTAGACATAATAGCAACAGACATACAAACTAAAGAATTACAAAAAC
AAATTACAAAAATTCAAAATTTTCGGGTTTATTACAGGGACAGCAGAAATCCACTTTGGAAAGGACCA
GCAAAGCTCCTCTGGAAAGGTGAAGGGGCAGTAGTAATACAAGATAATAGTGACATAAAAGTAGTGC
CAAGAAGAAAAGCAAAGATCATTAGGGATTATGGAAAACAGATGGCAGGTGATGATTGTGTGGCAAG
TAGACAGGATGAGGATTAGAACATGGAAAAGTTTAGTAAAACACCATAAGGAGGAGATATGAGGGAC
AATTGGAGAAGTGAATTATATAAATATAAAGTAGTAAAAATTGAACCATTAGGAGTAGCACCCACCAA
GGCAAAGAGAAGAGTGGTGCAGAGAGAAAAAAGAGCAGTGGGAATAGGAGCTTTGTTCCTTGGGTT
CTTGGGAGCAGCAGGAAGCACTATGGGCGCAGCGTCAATGACGCTGACGGTACAGGCCAGACAATT
ATTGTCTGGTATAGTGCAGCAGCAGAACAATTTGCTGAGGGCTATTGAGGCGCAACAGCATCTGTTG
CAACTCACAGTCTGGGGCATCAAGCAGCTCCAGGCAAGAATCCTGGCTGTGGAAAGATACCTAAAGG
ATCAACAGCTCCTGGGGATTTGGGGTTGCTCTGGAAAACTCATTTGCACCACTGCTGTGCCTTGGAAT
GCTAGTTGGAGTAATAAATCTCTGGAACAGATTTGGAATCACACGACCTGGATGGAGTGGGACAGAG
AAATTAACAATTACACAAGCTTAATACACTCCTTAATTGAAGAATCGCAAAACCAGCAAGAAAAGAATG
AACAAGAATTATTGGAATTAGATAAATGGGCAAGTTTGTGGAATTGGTTTAACATAACAAATTGGCTGT
GGTATATAAAATTATTCATAATGATAGTAGGAGGCTTGGTAGGTTTAAGAATAGTTTTTGCTGTACTTT
CTATAGTGAATAGAGTTAGGCAGGGATATTCACCATTATCGTTTCAGACCCACCTCCCAACCCCGAGG
GGACCGAGCTCAAGCTTCGAACGCGTGGGGATCCTCTAGAGTCGAGCTCGCGAGGATCATCACCGG
TGCTAGCCGGAGCCAGAAGCACCATAAGGGACATGATAAGGGAGCCAGCAGACCTCTGATCTCTTCC
TGAATGCTAATCTTAAACATCCTGAGGAAGAATGGGACTTCCATTTGGGGTGGGCCTATGATAGGGTA
ATAAGACAGTAGTGAATATCAAGCTACAAAAAGCCCCCTTTCAAATTCTTCTCAGTCCTAACTTTTCAT
ACTAAGCCCAGTCCTTCCAAAGCAGACTGTGAAAGAGTGATAGTTCCGGGAGACTAGCACCGGCTAG
CCGAGCTTGGAACACTTTCCCTTCATTAAGAACCATCCTTGCTACTCAGCTGCAATCAATCCAGCCCCC
AGGTCTTCACTGAACCTTTTCCCATCTCTTCCAAAACATCTGTTTCTGAGAAGTCCTGTCCTATAGAGG
TCTTTCTTCCCACCGGATTTCTCCTACACCATTTACTCCCACTTGCAGAACTCCCGTGTACAAGTGTCT
TTACTGCTTTTATTTGCTCAACAAAATGCACATCTCATATAAAAATAAATGAGGAGCATGCACACACCA
CAAACACAAACAGGCATGCAGAAATACACATACACACTTCCCTCAATATAAACCCTTTGTGGCTCATAT
ATTTAAAAAGATGTAAAAAAAAGAGCTGAAGAAAATCATGTGTGATCTCTCAGCAGAATAGATTTATTA
TTTGTATTGCTTGCAGAATAAAGCCTATCCTTGAAAGCTCTGAATCATGGGCAAGAGGCTCAGTGGTA
TCTGGAGGACAGGGCACTGGCCACTGCAGTCACCATCTTCTGCCAGGAAGCCTGCACCTCAGGGGTG
AATTCTTTGCCAAAGTGAATGGCCAGCACGGTGACCAGCACGTTGCCCAGGAGCTGTGGGAGGAAGA
TAAGAGATATGAACATGATTAGCAAAAGGGCCTAGCTTGGACTCAGAATAATCCAGCCTTATCCCAAC
CATAAAATAAAAGCAGAATGGTAGCTGGATTGTAGCTGCTATTAGCAATATGAAACCTCTTACATCAGT
TACAATTTATATGCAGAAATATTTATATGCAGAAATATTGCTATTGCCTTAACCCAGAAATTATCACTGT
TATTCTTTAGAATGGTGCAAAGAGGCATGATACATTGTATCATTATTGCCCTGAAAGAAAGAGATTAG
GGAAAGTATTAGAAATAAGATAAACAAAAAAGTATATTAAAAGAAGAAAGCATTTTTTAAAATTACAAA
TGCAAAATTACCCTGATTTGGTCAATATGTGTACCCTGTTACTTCTCCCCTTCCTATGACATGAACTTA
ACCATAGAAAAGAAGGGGAAAGAAAACATCAAGGGTCCCATAGACTCACCTTGAAGTTCTCAGGATC
CACATGCAGCTTGTCACAGTGCAGTTCACTCAGCTGGGCAAAGGTGCCCTTGAGATCATCCAGGTGC
TTTATGGCATCTCCCAAGGAAGTCAGCACCTTCTTGCCATGTGCCTTGACTTTGGGGTTGCCCATGAT
GGCAGAGGCAGAGGACAGGTTGCCAAAGCTGTCAAAGAACCTCTGGGTCCATGGGTAGACAACCAG
GAGCCTGTGAGATTGACAAGAACAGTTTGACAGTCAGAAGGTGCCACAAATCCTGAGAAGCAACCTG
GACTTTTGCCAGGCACAGGGTCCTTCCTTCCCTCCCTTGTCCTGGTCACCAGAGCCTACCTTCCCAGG
GTTTCTCCTCCAGCATCTTCCACATTCACCTTGTCCCACAGGCTTGTGATAGTAGCCTTGTCCTCCTCT
GTGAAATGACCCATGGTGTCTGTTTGAGGTTGCTAGTGAACACAGTTGTGTCAGAAGCAAATGTAAGC
AATAGATGGCTCTGCCCTGACTTTTATGCCCAGCCCTGGCTCCTGCCCTCCCTGCTCCTGGGAGTAGA
TTGGCCAACCCTAGGGTGTGGCTCCACAGGGTGAGGTCTAAGTGATGACAGCCGTACCTGTCCTTGG
CTCTTCTGGCACTGGCTTAGGAGTTGGACTTCAAACCCTCAGCCCTCCCTCTAAGATATATCTCTTGG
CCCCATACCATCAGTACAAATTGCTACTAAAAACATCCTCCTTTGCAAGTGTATTTACGACGGTATCGA
TGTATGTGAGCATGTGTCCTCTAACAGCACAGGCCTTTTGCCACCTAGCTGTCCAGGGGTGCCTTAAA
ATGGCAAACAAGGTTTGTTTTCTTTTCCTGTTTTCATGCCTTCCTCTTCCATATCCTTGTTTCATATTAAT
ACATGTGTATAGATCCTAAAAATCTATACACATGTATTAATAAAGCCTGATTCTGCCGCTTCTAGGTAT
AGAGGCCACCTGCAAGATAAATATTTGATTCACAATAACTAATCATTCTATGGCAATTGATAACAACAA
ATATATATATATATATATATATACGTATATGTGTATATATATATATATATTCAGGAAATAATATATTCTAG
AATATGTCACATTCTGTCTCAGGCATCCATTTTCTTTATGATGCCGTTTGAGGTGGAGTTTTAGTCAGG
TGGTCAGCTTCTCCTTTTTTTTGCCATCTGCCCTGTAAGCATCCTGCTGGGGACCCAGATAGGAGTCA
TCACTCTAGGCTGAGAACATCTGGGCACACACCCTAAGCCTCAGCATGACTCATCATGACTCAGCATT
GCTGTGCTTGAGCCAGAAGGTTTGCTTAGAAGGTTACACAGAACCAGAAGGCGGGGGTGGGGCACT
GACCCCGACAGGGGCCTGGCCAGAACTGCTCATGCTTGGACTATGGGAGGTCACTAATGGAGACAC
ACAGAAATGTAACAGGAACTAAGGGAATTCCGGTGCCCTGCTTAGGAGCTTAATCTTTAATGAAAGCT
AAGCTTTCATTAAAAAAAGTCTAACCAGCTGCATTCGACTTTGACTGCAGCAGCTGGTTAGAAGGTTC
TACTGGAGGAGGGTCCCAGCCCATTGCTAAATTAACATCAGGCTCTGAGACTGGCAGTATATCTCTAA
CAGTGGTTGATGCTATCTTCTGGAACTTGCCTGCTACATTGAGACCACTGACCCATACATAGGAAGCC
CATAGCTCTGTCCTGAACTGTTAGGCCACTGGTCCAGAGAGTGTGCATCTCCTTTGATCCTCATAATA
ACCCTATGAGATAGACACAATTATTACTCTTACTTTATAGATGATGATCCTGAAAACATAGGAGTCAAG
GCACTTGCCCCTAGCTGGGGGTATAGGGGAGCAGTCCCATGTAGTAGTAGAATGAAAAATGCTGCTA
TGCTGTGCCTCCCCCACCTTTCCCATGTCTGCCCTCTACTCATGGTCTATCTCTCCTGGCTCCTGGGA
GTCATGGACTCCACCCAGCACCACCAACCTGACCTAACCACCTATCTGAGCCTGCCAGCCTATAACCC
ATCTGGGCCCTGATAGCTGGTGGCCAGCCCTGACCCCACCCCACCCTCCCTGGAACCTCTGATAGAC
ACATCTGGCACACCAGCTCGCAAAGTCACCGTGAGGGTCTTGTGTTTGCTGAGTCAAAATTCCTTGAA
ATCCAAGTCCTTAGAGACTCCTGCTCCCAAATTTACAGTCATAGACTTCTTCATGGCTGTCTCCTTTAT
CCACAGAATGATTCCTTTGCTTCATTGCCCCATCCATCTGATCCTCCTCATCAGTGCAGCACAGGGCC
CATGAGCAGTAGCTGCAGAGTCTCACATAGGTCTGGCACTGCCTCTGACATGTCCGACCTTAGGCAA
ATGCTTGACTCTTCTGAGCTCGGATCCCTTGAGCTCAGGAGGTCAAGGCTGCAGTGAGACATGATCTT
GCCACTGCACTCCAGCCTGGACAGCAGAGTGAAACCTTGCCTCACGAAACAGAATACAAAAACAAAC
AAACAAAAAACTGCTCCGCAATGCGCTTCCTTGATGCTCTACCACATAGGTCTGGGTACTTTGTACAC
ATTATCTCATTGCTGTTCATAATTGTTAGATTAATTTTGTAATATTGATATTATTCCTAGAAAGCTGAGG
CCTCAAGATGATAACTTTTATTTTCTGGACTTGTAATAGCTTTCTCTTGTATTCACCATGTTGTAACTTT
CTTAGAGTAGTAACAATATAAAGTTATTGTGAGTTTTTGCAAACACAGCAAACACAACGACCCATATAG
ACATTGATGTGAAATTGTCTATTGTCAATTTATGGGAAAACAAGTATGTACTTTTTCTACTAAGCCATT
GAAACAGGAATAACAGAACAAGATTGAAAGAATACATTTTCCGAAATTACTTGAGTATTATACAAAGAC
AAGCACGTGGACCTGGGAGGAGGGTTATTGTCCATGACTGGTGTGTGGAGACAAATGCAGGTTTATA
ATAGATGGGATGGCATCTAGCGCAATGACTTTGCCATCACTTTTAGAGAGCTCTTGGGGGCCCCAGTA
CACAAGAGGGGACGCAGGGTATATGTAGACATCTCATTCTTTTTCTTAGTGTGAGAATAAGAATAGCC
ATGACCTGAGTTTATAGACAATGAGCCCTTTTCTCTCTCCCACTCAGCAGCTATGAGATGGCTTGCCCT
GCCTCTCTACTAGGCTGACTCACTCCAAGGCCCAGCAATGGGCAGGGCTCTGTCAGGGCTTTGATAG
CACTATCTGCAGAGCCAGGGCCGAGAAGGGGTGGACTCCAGAGACTCTCCCTCCCATTCCCGAGCAG
GGTTTGCTTATTTATGCATTTAAATGATATATTTATTTTAAAAGAAATAACAGGAGACTGCCCAGCCCT
GGCTGTGACATGGAAACTATGTAGAATATTTTGGGTTCCATTTTTTTTTCCTTCTTTCAGTTAGAGGAA
AAGGGGCTCACTGCACATACACTAGACAGAAAGTCAGGAGCTTTGAATCCAAGCCTGATCATTTCCAT
GTCATACTGAGAAAGTCCCCACCCTTCTCTGAGCCTCAGTTTCTCTTTTTATAAGTAGGAGTCTGGAGT
AAATGATTTCCAATGGCTCTCATTTCAATACAAAATTTCCGTTTATTAAATGCATGAGCTTCCGTTACTC
CAAGACTGAGAAGGAAATTGAACCTGAGACTCATTGACTGGCAAGATGTCCCCAGAGGCTCTCATTCA
GCAATAAAATTCTCACCTTCACCCAGGCCCACTAGTGTCAGATTTGCATGCGTTCGCGTATCGACGTG
CAGTATTTAGCATGCCCCACCCATCTGCAAGGCATTCTGGATAGTGTCAAAACAGCCGGAAATCAAGT
CCGTTTATCTCAAACTTTAGCATTTTGGGAATAAATGATATTTGCTATGCTGGTTAAATTAGATTTTAGT
TAAATTTCCTGCTGAAGCTCTAGTACGATAAGTAACTTGACCTAAGTGTAAAGTTGAGATTTCCTTCAG
GTTTATATAGCTTGTGCGCCGCCTGGGTACCTCAGGATATGCCCTTGACTATTTGTCCGACATAGTCA
AGGGCATATCCTTTTTTGTGCGGCCGCATCGATGCCGTAGTACCTTTAAGACCAATGACTTACAAGGC
AGCTGTAGATCTTAGCCACTTTTTAAAAGAAAAGGGGGGACTGGAAGGGCTAATTCACTCCCAAAGAA
GACAAGATCCCTGCAGGCATTCAAGGCCAGGCTGGATGTGGCTCTGGGCAGCCTGGGCTGCTGGTT
GATGACCCTGCACATAGCAGGGGGTTGGATCTGGATGAGCACTGTGCTCCTTTGCAACCCAGGCCGT
TCTATGATTCTGTCATTCTAAATCTCTCTTTCAGCCTAAAGCTTTTTCCCCGTATCCCCCCIGGTGTCTG
CIGGCTCAAAGAGCAGCGAGAAGCGTTCAGAGGAAAGCGATCCCGTGCCACCTTCCCCGTGCCCGG
GCTGTCCCCGCACGCTGCCGGCTCGGGGATGCGGGGGGAGCGCCGGACCGGAGCGGAGCCCCGG
GCGGCTCGCTGCTGCCCCCTAGCGGGGGAGGGACGTAATTACATCCCTGGGGGCTTTGGGGGGGG
GCTGTCCCCGTGAGCTCCCCAGATCTGCTTTTTGCCTGTACTGGGTCTCTCTGGTTAGACCAGATCTG
AGCCTGGGAGCTCTCTGGCTAACTAGGGAACCCACTGCTTAAGCCTCAATAAAGCTTCAGCTGCTCGA
GCTAGCAGATCTTTTTCCCTCTGCCAAAAATTATGGGGACATCATGAAGCCCCTTGAGCATCTGACTT
CTGGCTAATAAAGGAAATTTATTTTCATTGCAATAGTGTGTTGGAATTTTTTGTGTCTCTCACTCGGAA
GGACATATGGGAGGGCAAATCATTTAAAACATCAGAATGAGTATTTGGTTTAGAGTTTGGCAACATAT
GCCCATATGCTGGCTGCCATGAACAAAGGTTGGCTATAAAGAGGTCATCAGTATATGAAACAGCCCC
CTGCTGTCCATTCCTTATTCCATAGAAAAGCCTTGACTTGAGGTTAGATTTTTTTTATATTTTGTTTTGT
GTTATTTTTTTCTTTAACATCCCTAAAATTTTCCTTACATGTTTTACTAGCCAGATTTTTCCTCCTCTCCT
GACTACTCCCAGTCATAGCTGTCCCTCTTCTCTTATGGAGATCCCTCGACCTGCAGCCCAAGCTTGGC
GTAATCATGGTCATAGCTGTTTCCTGTGTGAAATTGTTATCCGCTCACAATTCCACACAACATACGAGC
CGGAAGCATAAAGTGTAAAGCCTGGGGTGCCTAATGAGTGAGCTAACTCACATTAATTGCGTTGCGC
TCACTGCCCGCTTTCCAGTCGGGAAACCTGTCGTGCCAGCGGATCCGCATCTCAATTAGTCAGCAACC
ATAGTCCCGCCCCTAACTCCGCCCATCCCGCCCCTAACTCCGCCCAGTTCCGCCCATTCTCCGCCCCA
TGGCTGACTAATTTTTTTTATTTATGCAGAGGCCGAGGCCGCCTCGGCCTCTGAGCTATTCCAGAAGT
AGTGAGGAGGCTTTTTTGGAGGCCTAGGCTTTTGCAAAAAGCTGTCGACTGCAGAGGCCTGCATGCA
AGCTTGGCGTAATCATGGTCATAGCTGTTTCCTGTGTGAAATTGTTATCCGCTCACAATTCCACACAAC
ATACGAGCCGGAAGCATAAAGTGTAAAGCCTGGGGTGCCTAATGAGTGAGCTAACTCACATTAATTG
CGTTGCGCTCACTGCCCGCTTTCCAGTCGGGAAACCTGTCGTGCCAGCTGCATTAATGAATCGGCCA
ACGCGCGGGGAGAGGCGGTTTGCGTATTGGGCGCTCTTCCGCTTCCTCGCTCACTGACTCGCTGCGC
TCGGTCGTTCGGCTGCGGCGAGCGGTATCAGCTCACTCAAAGGCGGTAATACGGTTATCCACAGAAT
CAGGGGATAACGCAGGAAAGAACATGTGAGCAAAAGGCCAGCAAAAGGCCAGGAACCGTAAAAAGG
CCGCGTTGCTGGCGTTTTTCCATAGGCTCCGCCCCCCTGACGAGCATCACAAAAATCGACGCTCAAGT
CAGAGGTGGCGAAACCCGACAGGACTATAAAGATACCAGGCGTTTCCCCCTGGAAGCTCCCTCGTGC
GCTCTCCTGTTCCGACCCTGCCGCTTACCGGATACCTGTCCGCCTTTCTCCCTTCGGGAAGCGTGGCG
CTTTCTCATAGCTCACGCTGTAGGTATCTCAGTTCGGTGTAGGTCGTTCGCTCCAAGCTGGGCTGTGT
GCACGAACCCCCCGTTCAGCCCGACCGCTGCGCCTTATCCGGTAACTATCGTCTTGAGTCCAACCCG
GTAAGACACGACTTATCGCCACTGGCAGCAGCCACTGGTAACAGGATTAGCAGAGCGAGGTATGTAG
GCGGTGCTACAGAGTTCTTGAAGTGGTGGCCTAACTACGGCTACACTAGAAGAACAGTATTTGGTATC
TGCGCTCTGCTGAAGCCAGTTACCTTCGGAAAAAGAGTTGGTAGCTCTTGATCCGGCAAACAAACCAC
CGCTGGTAGCGGTGGTTTTTTTGTTTGCAAGCAGCAGATTACGCGCAGAAAAAAAGGATCTCAAGAA
GATCCTTTGATCTTTTCTACGGGGTCTGACGCTCAGTGGAACGAAAACTCACGTTAAGGGATTTTGGT
CATGAGATTATCAAAAAGGATCTTCACCTAGATCCTTTTAAATTAAAAATGAAGTTTTAAATCAATCTAA
AGTATATATGAGTAAACTTGGTCTGACAGTTACCAATGCTTAATCAGTGAGGCACCTATCTCAGCGAT
CTGTCTATTTCGTTCATCCATAGTTGCCTGACTCCCCGTCGTGTAGATAACTACGATACGGGAGGGCT
TACCATCTGGCCCCAGTGCTGCAATGATACCGCGAGACCCACGCTCACCGGCTCCAGATTTATCAGC
AATAAACCAGCCAGCCGGAAGGGCCGAGCGCAGAAGTGGTCCTGCAACTTTATCCGCCTCCATCCAG
TCTATTAATTGTTGCCGGGAAGCTAGAGTAAGTAGTTCGCCAGTTAATAGTTTGCGCAACGTTGTTGC
CATTGCTACAGGCATCGTGGTGTCACGCTCGTCGTTTGGTATGGCTTCATTCAGCTCCGGTTCCCAAC
GATCAAGGCGAGTTACATGATCCCCCATGTTGTGCAAAAAAGCGGTTAGCTCCTTCGGTCCTCCGATC
GTTGTCAGAAGTAAGTTGGCCGCAGTGTTATCACTCATGGTTATGGCAGCACTGCATAATTCTCTTAC
TGTCATGCCATCCGTAAGATGCTTTTCTGTGACTGGTGAGTACTCAACCAAGTCATTCTGAGAATAGT
GTATGCGGCGACCGAGTTGCTCTTGCCCGGCGTCAATACGGGATAATACCGCGCCACATAGCAGAAC
TTTAAAAGTGCTCATCATTGGAAAACGTTCTTCGGGGCGAAAACTCTCAAGGATCTTACCGCTGTTGA
GATCCAGTTCGATGTAACCCACTCGTGCACCCAACTGATCTTCAGCATCTTTTACTTTCACCAGCGTTT
CTGGGTGAGCAAAAACAGGAAGGCAAAATGCCGCAAAAAAGGGAATAAGGGCGACACGGAAATGTT
GAATACTCATACTCTTCCTTTTTCAATATTATTGAAGCATTTATCAGGGTTATTGTCTCATGAGCGGATA
CATATTTGAATGTATTTAGAAAAATAAACAAATAGGGGTTCCGCGCACATTTCCCCGAAAAGTGCCAC
CTGACGTCTAAGAAACCATTATTATCATGACATTAACCTATAAAAATAGGCGTATCACGAGGCCCTTTC
GTCTCGCGCGTTTCGGTGATGACGGTGAAAACCTCTGACACATGCAGCTCCCGGAGACGGTCACAGC
TTGTCTGTAAGCGGATGCCGGGAGCAGACAAGCCCGTCAGGGCGCGTCAGCGGGTGTTGGCGGGTG
TCGGGGCTGGCTTAACTATGCGGCATCAGAGCAGATTGTACTGAGAGTGCACCATATGCGGTGTGAA
ATACCGCACAGATGCGTAAGGAGAAAATACCGCATCAGGCGCCATTCGCCATTCAGGCTGCGCAACT
GTTGGGAAGGGCGATCGGTGCGGGCCTCTTCGCTATTACGCCAGCTGGCGAAAGGGGGATGTGCTG
CAAGGCGATTAAGTTGGGTAACGCCAGGGTTTTCCCAGTCACGACGTTGTAAAACGACGGCCAGTGA
ATTC
pCalH11 TL20c rGbGM G3320A 7SKsh734 (SEQ ID NO: 112)
GGCCGCCTCGGCCAAACAGCCCTTGAGTTTACCACTCCCTATCAGTGATAGAGAAAAGTGAAAGTCG
AGTTTACCACTCCCTATCAGTGATAGAGAAAAGTGAAAGTCGAGTTTACCACTCCCTATCAGTGATAG
AGAAAAGTGAAAGTCGAGTTTACCACTCCCTATCAGTGATAGAGAAAAGTGAAAGTCGAGTTTACCAG
TCCCTATCAGTGATAGAGAAAAGTGAAAGTCGAGTTTACCACTCCCTATCAGTGATAGAGAAAAGTGA
AAGTCGAGTTTACCACTCCCTATCAGTGATAGAGAAAAGTGAAAGTCGAGCTCGCCATGGGAGGCGT
GGCCTGGGGGGACTGGGGAGTGGCGAGCCCTCAGATCCTGCATATAAGCAGCTGCTTTTTGCCTGT
ACTGGGTCTCTCTGGTTAGACCAGATCTGAGCCTGGGAGCTCTCTGGCTAACTAGGGAACCCACTGC
TTAAGCCTCAATAAAGCTTGCCTTGAGTGCTTCAAGTAGTGTGTGCCCGTCTGTTGTGTGACTCTGGT
AACTAGAGATCCCTCAGACCCTTTTAGTCAGTGTGGAAAATCTCTAGCAGTGGCGCCCGAACAGGGA
CTTGAAAGCGAAAGGGAAACCAGAGGAGCTCTCTCGACGCAGGACTCGGCTTGCTGAAGCGCGCAC
GGCAAGAGGCGAGGGGCGGCGACTGGTGAGTACGCCAAAAATTTTGACTAGCGGAGGCTAGAAGGA
GAGAGATGGGTGCGAGAGCGTCAGTATTAAGCGGGGGAGAATTAGATCGCGATGGGAAAAAATTCG
GTTAAGGCCAGGGGGAAAGAAAAAATATAAATTAAAACATATAGTATGGGCAAGCAGGGAGCTAGAA
CGATTCGCAGTTAATACTGGCCTGTTAGAAACATCAGAAGGCTGTAGACAAATACTGGGACAGCTACA
ACCATCCCTTCAGACAGGATCAGAAGAACTTAGATCATTATATAATACAGTAGCAACCCTCTATTGTGT
GCATCAAAGGATAGAGATAAAAGACACCAAGGAAGCTTTAGACAAGATAGAGGAAGAGCAAAACAAA
AGTAAGAAAAAAGCACAGCAAGCAGCAGGATCTTCAGACCTGGAAATTCCCTACAATCCCCAAAGTCA
AGGAGTAGTAGAATCTATGAATAAAGAATTAAAGAAAATTATAGGACAGGTAAGAGATCAGGCTGAAC
ATCTTAAGACAGCAGTACAAATGGCAGTATTCATCCACAATTTTAAAAGAAAAGGGGGGATTGGGGG
GTACAGTGCAGGGGAAAGAATAGTAGACATAATAGCAACAGACATACAAACTAAAGAATTACAAAAAC
AAATTACAAAAATTCAAAATTTTCGGGTTTATTACAGGGACAGCAGAAATCCACTTTGGAAAGGACCA
GCAAAGCTCCTCTGGAAAGGTGAAGGGGCAGTAGTAATACAAGATAATAGTGACATAAAAGTAGTGC
CAAGAAGAAAAGCAAAGATCATTAGGGATTATGGAAAACAGATGGCAGGTGATGATTGTGTGGCAAG
TAGACAGGATGAGGATTAGAACATGGAAAAGTTTAGTAAAACACCATAAGGAGGAGATATGAGGGAC
AATTGGAGAAGTGAATTATATAAATATAAAGTAGTAAAAATTGAACCATTAGGAGTAGCACCCACCAA
GGCAAAGAGAAGAGTGGTGCAGAGAGAAAAAAGAGCAGTGGGAATAGGAGCTTTGTTCCTTGGGTT
CTTGGGAGCAGCAGGAAGCACTATGGGCGCAGCGTCAATGACGCTGACGGTACAGGCCAGACAATT
ATTGTCTGGTATAGTGCAGCAGCAGAACAATTTGCTGAGGGCTATTGAGGCGCAACAGCATCTGTTG
CAACTCACAGTCTGGGGCATCAAGCAGCTCCAGGCAAGAATCCTGGCTGTGGAAAGATACCTAAAGG
ATCAACAGCTCCTGGGGATTTGGGGTTGCTCTGGAAAACTCATTTGCACCACTGCTGTGCCTTGGAAT
GCTAGTTGGAGTAATAAATCTCTGGAACAGATTTGGAATCACACGACCTGGATGGAGTGGGACAGAG
AAATTAACAATTACACAAGCTTAATACACTCCTTAATTGAAGAATCGCAAAACCAGCAAGAAAAGAATG
AACAAGAATTATTGGAATTAGATAAATGGGCAAGTTTGTGGAATTGGTTTAACATAACAAATTGGCTGT
GGTATATAAAATTATTCATAATGATAGTAGGAGGCTTGGTAGGTTTAAGAATAGTTTTTGCTGTACTTT
CTATAGTGAATAGAGTTAGGCAGGGATATTCACCATTATCGTTTCAGACCCACCTCCCAACCCCGAGG
GGACCGAGCTCAAGCTTCGAACGCGTGGGGATCCTCTAGAGTCGAGCTCGCGAGGATCATCACCGG
TGCTAGCCGGAGCCAGAAGCACCATAAGGGACATGATAAGGGAGCCAGCAGACCTCTGATCTCTTCC
TGAATGCTAATCTTAAACATCCTGAGGAAGAATGGGACTTCCATTTGGGGTGGGCCTATGATAGGGTA
ATAAGACAGTAGTGAATATCAAGCTACAAAAAGCCCCCTTTCAAATTCTTCTCAGTCCTAACTTTTCAT
ACTAAGCCCAGTCCTTCCAAAGCAGACTGTGAAAGAGTGATAGTTCCGGGAGACTAGCACCGGCTAG
CCGAGCTTGGAACACTTTCCCTTCATTAAGAACCATCCTTGCTACTCAGCTGCAATCAATCCAGCCCCC
AGGTCTTCACTGAACCTTTTCCCATCTCTTCCAAAACATCTGTTTCTGAGAAGTCCTGTCCTATAGAGG
TCTTTCTTCCCACCGGATTTCTCCTACACCATTTACTCCCACTTGCAGAACTCCCGTGTACAAGTGTCT
TTACTGCTTTTATTTGCTCAACAAAATGCACATCTCATATAAAAATAAATGAGGAGCATGCACACACCA
CAAACACAAACAGGCATGCAGAAATACACATACACACTTCCCTCAATATAAACCCTTTGTGGCTCATAT
ATTTAAAAAGATGTAAAAAAAAGAGCTGAAGAAAATCATGTGTGATCTCTCAGCAGAATAGATTTATTA
TTTGTATTGCTTGCAGAATAAAGCCTATCCTTGAAAGCTCTGAATCATGGGCAAGAGGCTCAGTGGTA
TCTGGAGGACAGGGCACTGGCCACTGCAGTCACCATCTTCTGCCAGGAAGCCTGCACCTCAGGGGTG
AATTCTTTGCCAAAGTGAATGGCCAGCACGGTGACCAGCACGTTGCCCAGGAGCTGTGGGAGGAAGA
TAAGAGATATGAACATGATTAGCAAAAGGGCCTAGCTTGGACTCAGAATAATCCAGCCTTATCCCAAC
CATAAAATAAAAGCAGAATGGTAGCTGGATTGTAGCTGCTATTAGCAATATGAAACCTCTTACATCAGT
TACAATTTATATGCAGAAATATTTATATGCAGAAATATTGCTATTGCCTTAACCCAGAAATTATCACTGT
TATTCTTTAGAATGGTGCAAAGAGGCATGATACATTGTATCATTATTGCCCTGAAAGAAAGAGATTAG
GGAAAGTATTAGAAATAAGATAAACAAAAAAGTATATTAAAAGAAGAAAGCATTTTTTAAAATTACAAA
TGCAAAATTACCCTGATTTGGTCAATATGTGTACCCTGTTACTTCTCCCCTTCCTATGACATGAACTTA
ACCATAGAAAAGAAGGGGAAAGAAAACATCAAGGGTCCCATAGACTCACCTTGAAGTTCTCAGGATC
CACATGCAGCTTGTCACAGTGCAGTTCACTCAGCTGGGCAAAGGTGCCCTTGAGATCATCCAGGTGC
TTTATGGCATCTCCCAAGGAAGTCAGCACCTTCTTGCCATGTGCCTTGACTTTGGGGTTGCCCATGAT
GGCAGAGGCAGAGGACAGGTTGCCAAAGCTGTCAAAGAACCTCTGGGTCCATGGGTAGACAACCAG
GAGCCTGTGAGATTGACAAGAACAGTTTGACAGTCAGAAGGTGCCACAAATCCTGAGAAGCAACCTG
GACTTTTGCCAGGCACAGGGTCCTTCCTTCCCTCCCTTGTCCTGGTCACCAGAGCCTACCTTCCCAGG
GTTTCTCCTCCAGCATCTTCCACATTCACCTTGTCCCACAGGCTTGTGATAGTAGCCTTGTCCTCCTCT
GTGAAATGACCCATGGTGTCTGTTTGAGGTTGCTAGTGAACACAGTTGTGTCAGAAGCAAATGTAAGC
AATAGATGGCTCTGCCCTGACTTTTATGCCCAGCCCTGGCTCCTGCCCTCCCTGCTCCTGGGAGTAGA
TTGGCCAACCCTAGGGTGTGGCTCCACAGGGTGAGGTCTAAGTGATGACAGCCGTACCTGTCCTTGG
CTCTTCTGGCACTGGCTTAGGAGTTGGACTTCAAACCCTCAGCCCTCCCTCTAAGATATATCTCTTGG
CCCCATACCATCAGTACAAATTGCTACTAAAAACATCCTCCTTTGCAAGTGTATTTACGACGGTATCGA
TGTATGTGAGCATGTGTCCTCTAACAGCACAGGCCTTTTGCCACCTAGCTGTCCAGGGGTGCCTTAAA
ATGGCAAACAAGGTTTGTTTTCTTTTCCTGTTTTCATGCCTTCCTCTTCCATATCCTTGTTTCATATTAAT
ACATGTGTATAGATCCTAAAAATCTATACACATGTATTAATAAAGCCTGATTCTGCCGCTTCTAGGTAT
AGAGGCCACCTGCAAGATAAATATTTGATTCACAATAACTAATCATTCTATGGCAATTGATAACAACAA
ATATATATATATATATATATATACGTATATGTGTATATATATATATATATTCAGGAAATAATATATTCTAG
AATATGTCACATTCTGTCTCAGGCATCCATTTTCTTTATGATGCCGTTTGAGGTGGAGTTTTAGTCAGG
TGGTCAGCTTCTCCTTTTTTTTGCCATCTGCCCTGTAAGCATCCTGCTGGGGACCCAGATAGGAGTCA
TCACTCTAGGCTGAGAACATCTGGGCACACACCCTAAGCCTCAGCATGACTCATCATGACTCAGCATT
GCTGTGCTTGAGCCAGAAGGTTTGCTTAGAAGGTTACACAGAACCAGAAGGCGGGGGTGGGGCACT
GACCCCGACAGGGGCCTGGCCAGAACTGCTCATGCTTGGACTATGGGAGGTCACTAATGGAGACAC
ACAGAAATGTAACAGGAACTAAGGGAATTCCGGTGCCCTGCTTAGGAGCTTAATCTTTAATGAAAGCT
AAGCTTTCATTAAAAAAAGTCTAACCAGCTGCATTCGACTTTGACTGCAGCAGCTGGTTAGAAGGTTC
TACTGGAGGAGGGTCCCAGCCCATTGCTAAATTAACATCAGGCTCTGAGACTGGCAGTATATCTCTAA
CAGTGGTTGATGCTATCTTCTGGAACTTGCCTGCTACATTGAGACCACTGACCCATACATAGGAAGCC
CATAGCTCTGTCCTGAACTGTTAGGCCACTGGTCCAGAGAGTGTGCATCTCCTTTGATCCTCATAATA
ACCCTATGAGATAGACACAATTATTACTCTTACTTTATAGATGATGATCCTGAAAACATAGGAGTCAAG
GCACTTGCCCCTAGCTGGGGGTATAGGGGAGCAGTCCCATGTAGTAGTAGAATGAAAAATGCTGCTA
TGCTGTGCCTCCCCCACCTTTCCCATGTCTGCCCTCTACTCATGGTCTATCTCTCCTGGCTCCTGGGA
GTCATGGACTCCACCCAGCACCACCAACCTGACCTAACCACCTATCTGAGCCTGCCAGCCTATAACCC
ATCTGGGCCCTGATAGCTGGTGGCCAGCCCTGACCCCACCCCACCCTCCCTGGAACCTCTGATAGAC
ACATCTGGCACACCAGCTCGCAAAGTCACCGTGAGGGTCTTGTGTTTGCTGAGTCAAAATTCCTTGAA
ATCCAAGTCCTTAGAGACTCCTGCTCCCAAATTTACAGTCATAGACTTCTTCATGGCTGTCTCCTTTAT
CCACAGAATGATTCCTTTGCTTCATTGCCCCATCCATCTGATCCTCCTCATCAGTGCAGCACAGGGCC
CATGAGCAGTAGCTGCAGAGTCTCACATAGGTCTGGCACTGCCTCTGACATGTCCGACCTTAGGCAA
ATGCTTGACTCTTCTGAGCTCGGATCCCTTGAGCTCAGGAGGTCAAGGCTGCAGTGAGACATGATCTT
GCCACTGCACTCCAGCCTGGACAGCAGAGTGAAACCTTGCCTCACGAAACAGAATACAAAAACAAAC
AAACAAAAAACTGCTCCGCAATGCGCTTCCTTGATGCTCTACCACATAGGTCTGGGTACTTTGTACAC
ATTATCTCATTGCTGTTCATAATTGTTAGATTAATTTTGTAATATTGATATTATTCCTAGAAAGCTGAGG
CCTCAAGATGATAACTTTTATTTTCTGGACTTGTAATAGCTTTCTCTTGTATTCACCATGTTGTAACTTT
CTTAGAGTAGTAACAATATAAAGTTATTGTGAGTTTTTGCAAACACAGCAAACACAACGACCCATATAG
ACATTGATGTGAAATTGTCTATTGTCAATTTATGGGAAAACAAGTATGTACTTTTTCTACTAAGCCATT
GAAACAGGAATAACAGAACAAGATTGAAAGAATACATTTTCCGAAATTACTTGAGTATTATACAAAGAC
AAGCACGTGGACCTGGGAGGAGGGTTATTGTCCATGACTGGTGTGTGGAGACAAATGCAGGTTTATA
ATAGATGGGATGGCATCTAGCGCAATGACTTTGCCATCACTTTTAGAGAGCTCTTGGGGGCCCCAGTA
CACAAGAGGGGACGCAGGGTATATGTAGACATCTCATTCTTTTTCTTAGTGTGAGAATAAGAATAGCC
ATGACCTGAGTTTATAGACAATGAGCCCTTTTCTCTCTCCCACTCAGCAGCTATGAGATGGCTTGCCCT
GCCTCTCTACTAGGCTGACTCACTCCAAGGCCCAGCAATGGGCAGGGCTCTGTCAGGGCTTTGATAG
CACTATCTGCAGAGCCAGGGCCGAGAAGGGGTGGACTCCAGAGACTCTCCCTCCCATTCCCGAGCAG
GGTTTGCTTATTTATGCATTTAAATGATATATTTATTTTAAAAGAAATAACAGGAGACTGCCCAGCCCT
GGCTGTGACATGGAAACTATGTAGAATATTTTGGGTTCCATTTTTTTTTCCTTCTTTCAGTTAGAGGAA
AAGGGGCTCACTGCACATACACTAGACAGAAAGTCAGGAGCTTTGAATCCAAGCCTGATCATTTCCAT
GTCATACTGAGAAAGTCCCCACCCTTCTCTGAGCCTCAGTTTCTCTTTTTATAAGTAGGAGTCTGGAGT
AAATGATTTCCAATGGCTCTCATTTCAATACAAAATTTCCGTTTATTAAATGCATGAGCTTCCGTTACTC
CAAGACTGAGAAGGAAATTGAACCTGAGACTCATTGACTGGCAAGATGTCCCCAGAGGCTCTCATTCA
GCAATAAAATTCTCACCTTCACCCAGGCCCACTAGTGTCAGATTTGCATGCGTTCGCGTATCGACGTG
CAGTATTTAGCATGCCCCACCCATCTGCAAGGCATTCTGGATAGTGTCAAAACAGCCGGAAATCAAGT
CCGTTTATCTCAAACTTTAGCATTTTGGGAATAAATGATATTTGCTATGCTGGTTAAATTAGATTTTAGT
TAAATTTCCTGCTGAAGCTCTAGTACGATAAGTAACTTGACCTAAGTGTAAAGTTGAGATTTCCTTCAG
GTTTATATAGCTTGTGCGCCGCCTGGGTACCTCAGGATATGCCCTTGACTATTTGTCCGACATAGTCA
AGGGCATATCCTTTTTTGTGCGGCCGCATCGATGCCGTAGTACCTTTAAGACCAATGACTTACAAGGC
AGCTGTAGATCTTAGCCACTTTTTAAAAGAAAAGGGGGGACTGGAAGGGCTAATTCACTCCCAAAGAA
GACAAGATCCCTGCAGGCATTCAAGGCCAGGCTGGATGTGGCTCTGGGCAGCCTGGGCTGCTGGTT
GATGACCCTGCACATAGCAGGGGGTTGGATCTGGATGAGCACTGTGCTCCTTTGCAACCCAGGCCGT
TCTATGATTCTGTCATTCTAAATCTCTCTTTCAGCCTAAAGCTTTTTCCCCGTATCCCCCCAGGTGTCTG
CAGGCTCAAAGAGCAGCGAGAAGCGTTCAGAGGAAAGCGATCCCGTGCCACCTTCCCCGTGCCCGG
GCTGTCCCCGCACGCTGCCGGCTCGGGGATGCGGGGGGAGCGCCGGACCGGAGCGGAGCCCCGG
GCGGCTCGCTGCTGCCCCCTAGCGGGGGAGGGACGTAATTACATCCCTGGGGGCTTTGGGGGGGG
GCTGTCCCCGTGAGCTCCCCAGATCTGCTTTTTGCCTGTACTGGGTCTCTCTGGTTAGACCAGATCTG
AGCCTGGGAGCTCTCTGGCTAACTAGGGAACCCACTGCTTAAGCCTCAATAAAGCTTCAGCTGCTCGA
GCTAGCAGATCTTTTTCCCTCTGCCAAAAATTATGGGGACATCATGAAGCCCCTTGAGCATCTGACTT
CTGGCTAATAAAGGAAATTTATTTTCATTGCAATAGTGTGTTGGAATTTTTTGTGTCTCTCACTCGGAA
GGACATATGGGAGGGCAAATCATTTAAAACATCAGAATGAGTATTTGGTTTAGAGTTTGGCAACATAT
GCCCATATGCTGGCTGCCATGAACAAAGGTTGGCTATAAAGAGGTCATCAGTATATGAAACAGCCCC
CTGCTGTCCATTCCTTATTCCATAGAAAAGCCTTGACTTGAGGTTAGATTTTTTTTATATTTTGTTTTGT
GTTATTTTTTTCTTTAACATCCCTAAAATTTTCCTTACATGTTTTACTAGCCAGATTTTTCCTCCTCTCCT
GACTACTCCCAGTCATAGCTGTCCCTCTTCTCTTATGGAGATCCCTCGACCTGCAGCCCAAGCTTGGC
GTAATCATGGTCATAGCTGTTTCCTGTGTGAAATTGTTATCCGCTCACAATTCCACACAACATACGAGC
CGGAAGCATAAAGTGTAAAGCCTGGGGTGCCTAATGAGTGAGCTAACTCACATTAATTGCGTTGCGC
TCACTGCCCGCTTTCCAGTCGGGAAACCTGTCGTGCCAGCGGATCCGCATCTCAATTAGTCAGCAACC
ATAGTCCCGCCCCTAACTCCGCCCATCCCGCCCCTAACTCCGCCCAGTTCCGCCCATTCTCCGCCCCA
TGGCTGACTAATTTTTTTTATTTATGCAGAGGCCGAGGCCGCCTCGGCCTCTGAGCTATTCCAGAAGT
AGTGAGGAGGCTTTTTTGGAGGCCTAGGCTTTTGCAAAAAGCTGTCGACTGCAGAGGCCTGCATGCA
AGCTTGGCGTAATCATGGTCATAGCTGTTTCCTGTGTGAAATTGTTATCCGCTCACAATTCCACACAAC
ATACGAGCCGGAAGCATAAAGTGTAAAGCCTGGGGTGCCTAATGAGTGAGCTAACTCACATTAATTG
CGTTGCGCTCACTGCCCGCTTTCCAGTCGGGAAACCTGTCGTGCCAGCTGCATTAATGAATCGGCCA
ACGCGCGGGGAGAGGCGGTTTGCGTATTGGGCGCTCTTCCGCTTCCTCGCTCACTGACTCGCTGCGC
TCGGTCGTTCGGCTGCGGCGAGCGGTATCAGCTCACTCAAAGGCGGTAATACGGTTATCCACAGAAT
CAGGGGATAACGCAGGAAAGAACATGTGAGCAAAAGGCCAGCAAAAGGCCAGGAACCGTAAAAAGG
CCGCGTTGCTGGCGTTTTTCCATAGGCTCCGCCCCCCTGACGAGCATCACAAAAATCGACGCTCAAGT
CAGAGGTGGCGAAACCCGACAGGACTATAAAGATACCAGGCGTTTCCCCCTGGAAGCTCCCTCGTGC
GCTCTCCTGTTCCGACCCTGCCGCTTACCGGATACCTGTCCGCCTTTCTCCCTTCGGGAAGCGTGGCG
CTTTCTCATAGCTCACGCTGTAGGTATCTCAGTTCGGTGTAGGTCGTTCGCTCCAAGCTGGGCTGTGT
GCACGAACCCCCCGTTCAGCCCGACCGCTGCGCCTTATCCGGTAACTATCGTCTTGAGTCCAACCCG
GTAAGACACGACTTATCGCCACTGGCAGCAGCCACTGGTAACAGGATTAGCAGAGCGAGGTATGTAG
GCGGTGCTACAGAGTTCTTGAAGTGGTGGCCTAACTACGGCTACACTAGAAGAACAGTATTTGGTATC
TGCGCTCTGCTGAAGCCAGTTACCTTCGGAAAAAGAGTTGGTAGCTCTTGATCCGGCAAACAAACCAC
CGCTGGTAGCGGTGGTTTTTTTGTTTGCAAGCAGCAGATTACGCGCAGAAAAAAAGGATCTCAAGAA
GATCCTTTGATCTTTTCTACGGGGTCTGACGCTCAGTGGAACGAAAACTCACGTTAAGGGATTTTGGT
CATGAGATTATCAAAAAGGATCTTCACCTAGATCCTTTTAAATTAAAAATGAAGTTTTAAATCAATCTAA
AGTATATATGAGTAAACTTGGTCTGACAGTTACCAATGCTTAATCAGTGAGGCACCTATCTCAGCGAT
CTGTCTATTTCGTTCATCCATAGTTGCCTGACTCCCCGTCGTGTAGATAACTACGATACGGGAGGGCT
TACCATCTGGCCCCAGTGCTGCAATGATACCGCGAGACCCACGCTCACCGGCTCCAGATTTATCAGC
AATAAACCAGCCAGCCGGAAGGGCCGAGCGCAGAAGTGGTCCTGCAACTTTATCCGCCTCCATCCAG
TCTATTAATTGTTGCCGGGAAGCTAGAGTAAGTAGTTCGCCAGTTAATAGTTTGCGCAACGTTGTTGC
CATTGCTACAGGCATCGTGGTGTCACGCTCGTCGTTTGGTATGGCTTCATTCAGCTCCGGTTCCCAAC
GATCAAGGCGAGTTACATGATCCCCCATGTTGTGCAAAAAAGCGGTTAGCTCCTTCGGTCCTCCGATC
GTTGTCAGAAGTAAGTTGGCCGCAGTGTTATCACTCATGGTTATGGCAGCACTGCATAATTCTCTTAC
TGTCATGCCATCCGTAAGATGCTTTTCTGTGACTGGTGAGTACTCAACCAAGTCATTCTGAGAATAGT
GTATGCGGCGACCGAGTTGCTCTTGCCCGGCGTCAATACGGGATAATACCGCGCCACATAGCAGAAC
TTTAAAAGTGCTCATCATTGGAAAACGTTCTTCGGGGCGAAAACTCTCAAGGATCTTACCGCTGTTGA
GATCCAGTTCGATGTAACCCACTCGTGCACCCAACTGATCTTCAGCATCTTTTACTTTCACCAGCGTTT
CTGGGTGAGCAAAAACAGGAAGGCAAAATGCCGCAAAAAAGGGAATAAGGGCGACACGGAAATGTT
GAATACTCATACTCTTCCTTTTTCAATATTATTGAAGCATTTATCAGGGTTATTGTCTCATGAGCGGATA
CATATTTGAATGTATTTAGAAAAATAAACAAATAGGGGTTCCGCGCACATTTCCCCGAAAAGTGCCAC
CTGACGTCTAAGAAACCATTATTATCATGACATTAACCTATAAAAATAGGCGTATCACGAGGCCCTTTC
GTCTCGCGCGTTTCGGTGATGACGGTGAAAACCTCTGACACATGCAGCTCCCGGAGACGGTCACAGC
TTGTCTGTAAGCGGATGCCGGGAGCAGACAAGCCCGTCAGGGCGCGTCAGCGGGTGTTGGCGGGTG
TCGGGGCTGGCTTAACTATGCGGCATCAGAGCAGATTGTACTGAGAGTGCACCATATGCGGTGTGAA
ATACCGCACAGATGCGTAAGGAGAAAATACCGCATCAGGCGCCATTCGCCATTCAGGCTGCGCAACT
GTTGGGAAGGGCGATCGGTGCGGGCCTCTTCGCTATTACGCCAGCTGGCGAAAGGGGGATGTGCTG
CAAGGCGATTAAGTTGGGTAACGCCAGGGTTTTCCCAGTCACGACGTTGTAAAACGACGGCCAGTGA
ATTC
pCalH31 TL20c rGbGM 7SKsh734 400 1AT (SEQ ID NO: 113)
GGCCGCCTCGGCCAAACAGCCCTTGAGTTTACCACTCCCTATCAGTGATAGAGAAAAGTGAAAGTCG
AGTTTACCACTCCCTATCAGTGATAGAGAAAAGTGAAAGTCGAGTTTACCACTCCCTATCAGTGATAG
AGAAAAGTGAAAGTCGAGTTTACCACTCCCTATCAGTGATAGAGAAAAGTGAAAGTCGAGTTTACCAG
TCCCTATCAGTGATAGAGAAAAGTGAAAGTCGAGTTTACCACTCCCTATCAGTGATAGAGAAAAGTGA
AAGTCGAGTTTACCACTCCCTATCAGTGATAGAGAAAAGTGAAAGTCGAGCTCGCCATGGGAGGCGT
GGCCTGGGCGGGACTGGGGAGTGGCGAGCCCTCAGATCCTGCATATAAGCAGCTGCTTTTTGCCTGT
ACTGGGTCTCTCTGGTTAGACCAGATCTGAGCCTGGGAGCTCTCTGGCTAACTAGGGAACCCACTGC
TTAAGCCTCAATAAAGCTTGCCTTGAGTGCTTCAAGTAGTGTGTGCCCGTCTGTTGTGTGACTCTGGT
AACTAGAGATCCCTCAGACCCTTTTAGTCAGTGTGGAAAATCTCTAGCAGTGGCGCCCGAACAGGGA
CTTGAAAGCGAAAGGGAAACCAGAGGAGCTCTCTCGACGCAGGACTCGGCTTGCTGAAGCGCGCAC
GGCAAGAGGCGAGGGGCGGCGACTGGTGAGTACGCCAAAAATTTTGACTAGCGGAGGCTAGAAGGA
GAGAGATGGGTGCGAGAGCGTCAGTATTAAGCGGGGGAGAATTAGATCGCGATGGGAAAAAATTCG
GTTAAGGCCAGGGGGAAAGAAAAAATATAAATTAAAACATATAGTATGGGCAAGCAGGGAGCTAGAA
CGATTCGCAGTTAATACTGGCCTGTTAGAAACATCAGAAGGCTGTAGACAAATACTGGGACAGCTACA
ACCATCCCTTCAGACAGGATCAGAAGAACTTAGATCATTATATAATACAGTAGCAACCCTCTATTGTGT
GCATCAAAGGATAGAGATAAAAGACACCAAGGAAGCTTTAGACAAGATAGAGGAAGAGCAAAACAAA
AGTAAGAAAAAAGCACAGCAAGCAGCAGGATCTTCAGACCTGGAAATTCCCTACAATCCCCAAAGTCA
AGGAGTAGTAGAATCTATGAATAAAGAATTAAAGAAAATTATAGGACAGGTAAGAGATCAGGCTGAAC
ATCTTAAGACAGCAGTACAAATGGCAGTATTCATCCACAATTTTAAAAGAAAAGGGGGGATTGGGGG
GTACAGTGCAGGGGAAAGAATAGTAGACATAATAGCAACAGACATACAAACTAAAGAATTACAAAAAC
AAATTACAAAAATTCAAAATTTTCGGGTTTATTACAGGGACAGCAGAAATCCACTTTGGAAAGGACCA
GCAAAGCTCCTCTGGAAAGGTGAAGGGGCAGTAGTAATACAAGATAATAGTGACATAAAAGTAGTGC
CAAGAAGAAAAGCAAAGATCATTAGGGATTATGGAAAACAGATGGCAGGTGATGATTGTGTGGCAAG
TAGACAGGATGAGGATTAGAACATGGAAAAGTTTAGTAAAACACCATAAGGAGGAGATATGAGGGAC
AATTGGAGAAGTGAATTATATAAATATAAAGTAGTAAAAATTGAACCATTAGGAGTAGCACCCACCAA
GGCAAAGAGAAGAGTGGTGCAGAGAGAAAAAAGAGCAGTGGGAATAGGAGCTTTGTTCCTTGGGTT
CTTGGGAGCAGCAGGAAGCACTATGGGCGCAGCGTCAATGACGCTGACGGTACAGGCCAGACAATT
ATTGTCTGGTATAGTGCAGCAGCAGAACAATTTGCTGAGGGCTATTGAGGCGCAACAGCATCTGTTG
CAACTCACAGTCTGGGGCATCAAGCAGCTCCAGGCAAGAATCCTGGCTGTGGAAAGATACCTAAAGG
ATCAACAGCTCCTGGGGATTTGGGGTTGCTCTGGAAAACTCATTTGCACCACTGCTGTGCCTTGGAAT
GCTAGTTGGAGTAATAAATCTCTGGAACAGATTTGGAATCACACGACCTGGATGGAGTGGGACAGAG
AAATTAACAATTACACAAGCTTAATACACTCCTTAATTGAAGAATCGCAAAACCAGCAAGAAAAGAATG
AACAAGAATTATTGGAATTAGATAAATGGGCAAGTTTGTGGAATTGGTTTAACATAACAAATTGGCTGT
GGTATATAAAATTATTCATAATGATAGTAGGAGGCTTGGTAGGTTTAAGAATAGTTTTTGCTGTACTTT
CTATAGTGAATAGAGTTAGGCAGGGATATTCACCATTATCGTTTCAGACCCACCTCCCAACCCCGAGG
GGACCGAGCTCAAGCTTCGAACGCGTGGGGATCCTCTAGAGTCGAGCTCGCGAGGATCATCACCGG
TGCTAGCCGGAGCCAGAAGCACCATAAGGGACATGATAAGGGAGCCAGCAGACCTCTGATCTCTTCC
TGAATGCTAATCTTAAACATCCTGAGGAAGAATGGGACTTCCATTTGGGGTGGGCCTATGATAGGGTA
ATAAGACAGTAGTGAATATCAAGCTACAAAAAGCCCCCTTTCAAATTCTTCTCAGTCCTAACTTTTCAT
ACTAAGCCCAGTCCTTCCAAAGCAGACTGTGAAAGAGTGATAGTTCCGGGAGACTAGCACCGGCTAG
CCGAGCTTGGAACACTTTCCCTTCATTAAGAACCATCCTTGCTACTCAGCTGCAATCAATCCAGCCCCC
AGGTCTTCACTGAACCTTTTCCCATCTCTTCCAAAACATCTGTTTCTGAGAAGTCCTGTCCTATAGAGG
TCTTTCTTCCCACCGGATTTCTCCTACACCATTTACTCCCACTTGCAGAACTCCCGTGTACAAGTGTCT
TTACTGCTTTTATTTGCTCAACAAAATGCACATCTCATATAAAAATAAATGAGGAGCATGCACACACCA
CAAACACAAACAGGCATGCAGAAATACACATACACACTTCCCTCAATATAAACCCTTTGTGGCTCATAT
ATTTAAAAAGATGTAAAAAAAAGAGCTGAAGAAAATCATGTGTGATCTCTCAGCAGAATAGATTTATTA
TTTGTATTGCTTGCAGAATAAAGCCTATCCTTGAAAGCTCTGAATCATGGGCAAGAGGCTCAGTGGTA
TCTGGAGGACAGGGCACTGGCCACTGCAGTCACCATCTTCTGCCAGGAAGCCTGCACCTCAGGGGTG
AATTCTTTGCCAAAGTGAATGGCCAGCACGGTGACCAGCACGTTGCCCAGGAGCTGTGGGAGGAAGA
TAAGAGGTATGAACATGATTAGCAAAAGGGCCTAGCTTGGACTCAGAATAATCCAGCCTTATCCCAAC
CATAAAATAAAAGCAGAATGGTAGCTGGATTGTAGCTGCTATTAGCAATATGAAACCTCTTACATCAGT
TACAATTTATATGCAGAAATATTTATATGCAGAAATATTGCTATTGCCTTAACCCAGAAATTATCACTGT
TATTCTTTAGAATGGTGCAAAGAGGCATGATACATTGTATCATTATTGCCCTGAAAGAAAGAGATTAG
GGAAAGTATTAGAAATAAGATAAACAAAAAAGTATATTAAAAGAAGAAAGCATTTTTTAAAATTACAAA
TGCAAAATTACCCTGATTTGGTCAATATGTGTACCCTGTTACTTCTCCCCTTCCTATGACATGAACTTA
ACCATAGAAAAGAAGGGGAAAGAAAACATCAAGGGTCCCATAGACTCACCTTGAAGTTCTCAGGATC
CACATGCAGCTTGTCACAGTGCAGTTCACTCAGCTGGGCAAAGGTGCCCTTGAGATCATCCAGGTGC
TTTATGGCATCTCCCAAGGAAGTCAGCACCTTCTTGCCATGTGCCTTGACTTTGGGGTTGCCCATGAT
GGCAGAGGCAGAGGACAGGTTGCCAAAGCTGTCAAAGAACCTCTGGGTCCATGGGTAGACAACCAG
GAGCCTGTGAGATTGACAAGAACAGTTTGACAGTCAGAAGGTGCCACAAATCCTGAGAAGCAACCTG
GACTTTTGCCAGGCACAGGGTCCTTCCTTCCCTCCCTTGTCCTGGTCACCAGAGCCTACCTTCCCAGG
GTTTCTCCTCCAGCATCTTCCACATTCACCTTGTCCCACAGGCTTGTGATAGTAGCCTTGTCCTCCTCT
GTGAAATGACCCATGGTGTCTGTTTGAGGTTGCTAGTGAACACAGTTGTGTCAGAAGCAAATGTAAGC
AATAGATGGCTCTGCCCTGACTTTTATGCCCAGCCCTGGCTCCTGCCCTCCCTGCTCCTGGGAGTAGA
TTGGCCAACCCTAGGGTGTGGCTCCACAGGGTGAGGTCTAAGTGATGACAGCCGTACCTGTCCTTGG
CTCTTCTGGCACTGGCTTAGGAGTTGGACTTCAAACCCTCAGCCCTCCCTCTAAGATATATCTCTTGG
CCCCATACCATCAGTACAAATTGCTACTAAAAACATCCTCCTTTGCAAGTGTATTTACGACGGTATCGA
TGTATGTGAGCATGTGTCCTCTAACAGCACAGGCCTTTTGCCACCTAGCTGTCCAGGGGTGCCTTAAA
ATGGCAAACAAGGTTTGTTTTCTTTTCCTGTTTTCATGCCTTCCTCTTCCATATCCTTGTTTCATATTAAT
ACATGTGTATAGATCCTAAAAATCTATACACATGTATTAATAAAGCCTGATTCTGCCGCTTCTAGGTAT
AGAGGCCACCTGCAAGATAAATATTTGATTCACAATAACTAATCATTCTATGGCAATTGATAACAACAA
ATATATATATATATATATATATACGTATATGTGTATATATATATATATATTCAGGAAATAATATATTCTAG
AATATGTCACATTCTGTCTCAGGCATCCATTTTCTTTATGATGCCGTTTGAGGTGGAGTTTTAGTCAGG
TGGTCAGCTTCTCCTTTTTTTTGCCATCTGCCCTGTAAGCATCCTGCTGGGGACCCAGATAGGAGTCA
TCACTCTAGGCTGAGAACATCTGGGCACACACCCTAAGCCTCAGCATGACTCATCATGACTCAGCATT
GCTGTGCTTGAGCCAGAAGGTTTGCTTAGAAGGTTACACAGAACCAGAAGGCGGGGGTGGGGCACT
GACCCCGACAGGGGCCTGGCCAGAACTGCTCATGCTTGGACTATGGGAGGTCACTAATGGAGACAC
ACAGAAATGTAACAGGAACTAAGGGAATTCCGGTGCCCTGCTTAGGAGCTTAATCTTTAATGAAAGCT
AAGCTTTCATTAAAAAAAGTCTAACCAGCTGCATTCGACTTTGACTGCAGCAGCTGGTTAGAAGGTTC
TACTGGAGGAGGGTCCCAGCCCATTGCTAAATTAACATCAGGCTCTGAGACTGGCAGTATATCTCTAA
CAGTGGTTGATGCTATCTTCTGGAACTTGCCTGCTACATTGAGACCACTGACCCATACATAGGAAGCC
CATAGCTCTGTCCTGAACTGTTAGGCCACTGGTCCAGAGAGTGTGCATCTCCTTTGATCCTCATAATA
ACCCTATGAGATAGACACAATTATTACTCTTACTTTATAGATGATGATCCTGAAAACATAGGAGTCAAG
GCACTTGCCCCTAGCTGGGGGTATAGGGGAGCAGTCCCATGTAGTAGTAGAATGAAAAATGCTGCTA
TGCTGTGCCTCCCCCACCTTTCCCATGTCTGCCCTCTACTCATGGTCTATCTCTCCTGGCTCCTGGGA
GTCATGGACTCCACCCAGCACCACCAACCTGACCTAACCACCTATCTGAGCCTGCCAGCCTATAACCC
ATCTGGGCCCTGATAGCTGGTGGCCAGCCCTGACCCCACCCCACCCTCCCTGGAACCTCTGATAGAC
ACATCTGGCACACCAGCTCGCAAAGTCACCGTGAGGGTCTTGTGTTTGCTGAGTCAAAATTCCTTGAA
ATCCAAGTCCTTAGAGACTCCTGCTCCCAAATTTACAGTCATAGACTTCTTCATGGCTGTCTCCTTTAT
CCACAGAATGATTCCTTTGCTTCATTGCCCCATCCATCTGATCCTCCTCATCAGTGCAGCACAGGGCC
CATGAGCAGTAGCTGCAGAGTCTCACATAGGTCTGGCACTGCCTCTGACATGTCCGACCTTAGGCAA
ATGCTTGACTCTTCTGAGCTCGGATCCCTTGAGCTCAGGAGGTCAAGGCTGCAGTGAGACATGATCTT
GCCACTGCACTCCAGCCTGGACAGCAGAGTGAAACCTTGCCTCACGAAACAGAATACAAAAACAAAC
AAACAAAAAACTGCTCCGCAATGCGCTTCCTTGATGCTCTACCACATAGGTCTGGGTACTTTGTACAC
ATTATCTCATTGCTGTTCATAATTGTTAGATTAATTTTGTAATATTGATATTATTCCTAGAAAGCTGAGG
CCTCAAGATGATAACTTTTATTTTCTGGACTTGTAATAGCTTTCTCTTGTATTCACCATGTTGTAACTTT
CTTAGAGTAGTAACAATATAAAGTTATTGTGAGTTTTTGCAAACACAGCAAACACAACGACCCATATAG
ACATTGATGTGAAATTGTCTATTGTCAATTTATGGGAAAACAAGTATGTACTTTTTCTACTAAGCCATT
GAAACAGGAATAACAGAACAAGATTGAAAGAATACATTTTCCGAAATTACTTGAGTATTATACAAAGAC
AAGCACGTGGACCTGGGAGGAGGGTTATTGTCCATGACTGGTGTGTGGAGACAAATGCAGGTTTATA
ATAGATGGGATGGCATCTAGCGCAATGACTTTGCCATCACTTTTAGAGAGCTCTTGGGGGCCCCAGTA
CACAAGAGGGGACGCAGGGTATATGTAGACATCTCATTCTTTTTCTTAGTGTGAGAATAAGAATAGCC
ATGACCTGAGTTTATAGACAATGAGCCCTTTTCTCTCTCCCACTCAGCAGCTATGAGATGGCTTGCCCT
GCCTCTCTACTAGGCTGACTCACTCCAAGGCCCAGCAATGGGCAGGGCTCTGTCAGGGCTTTGATAG
CACTATCTGCAGAGCCAGGGCCGAGAAGGGGTGGACTCCAGAGACTCTCCCTCCCATTCCCGAGCAG
GGTTTGCTTATTTATGCATTTAAATGATATATTTATTTTAAAAGAAATAACAGGAGACTGCCCAGCCCT
GGCTGTGACATGGAAACTATGTAGAATATTTTGGGTTCCATTTTTTTTTCCTTCTTTCAGTTAGAGGAA
AAGGGGCTCACTGCACATACACTAGACAGAAAGTCAGGAGCTTTGAATCCAAGCCTGATCATTTCCAT
GTCATACTGAGAAAGTCCCCACCCTTCTCTGAGCCTCAGTTTCTCTTTTTATAAGTAGGAGTCTGGAGT
AAATGATTTCCAATGGCTCTCATTTCAATACAAAATTTCCGTTTATTAAATGCATGAGCTTCCGTTACTC
CAAGACTGAGAAGGAAATTGAACCTGAGACTCATTGACTGGCAAGATGTCCCCAGAGGCTCTCATTCA
GCAATAAAATTCTCACCTTCACCCAGGCCCACTAGTGTCAGATTTGCATGCGTTCGCGTATCGACGTG
CAGTATTTAGCATGCCCCACCCATCTGCAAGGCATTCTGGATAGTGTCAAAACAGCCGGAAATCAAGT
CCGTTTATCTCAAACTTTAGCATTTTGGGAATAAATGATATTTGCTATGCTGGTTAAATTAGATTTTAGT
TAAATTTCCTGCTGAAGCTCTAGTACGATAAGTAACTTGACCTAAGTGTAAAGTTGAGATTTCCTTCAG
GTTTATATAGCTTGTGCGCCGCCTGGGTACCTCAGGATATGCCCTTGACTATTTGTCCGACATAGTCA
AGGGCATATCCTTTTTTGTGCGGCCGCATCGATGCCGTAGTACCTTTAAGACCAATGACTTACAAGGC
AGCTGTAGATCTTAGCCACTTTTTAAAAGAAAAGGGGGGACTGGAAGGGCTAATTCACTCCCAAAGAA
GACAAGATCCCTGCAGGCATTCAAGGCCAGGCTGGATGTGGCTCTGGGCAGCCTGGGCTGCTGGTT
GATGACCCTGCACATAGCAGGGGGTTGGATCTGGATGAGCACTGTGCTCCTTTGCAACCCAGGCCGT
TCTATGATTCTGTCATTCTAAATCTCTCTTTCAGCCTAAAGCTTTTTCCCCGTATCCCCCCIGGTGTCTG
CAGGCTCAAAGAGCAGCGAGAAGCGTTCAGAGGAAAGCGATCCCGTGCCACCTTCCCCGTGCCCGG
GCTGTCCCCGCACGCTGCCGGCTCGGGGATGCGGGGGGAGCGCCGGACCGGAGCGGAGCCCCGG
GCGGCTCGCTGCTGCCCCCTAGCGGGGGAGGGACGTAATTACATCCCTGGGGGCTTTGGGGGGGG
GCTGTCCCCGTGAGCTCCCCAGATCTGCTTTTTGCCTGTACTGGGTCTCTCTGGTTAGACCAGATCTG
AGCCTGGGAGCTCTCTGGCTAACTAGGGAACCCACTGCTTAAGCCTCAATAAAGCTTCAGCTGCTCGA
GCTAGCAGATCTTTTTCCCTCTGCCAAAAATTATGGGGACATCATGAAGCCCCTTGAGCATCTGACTT
CTGGCTAATAAAGGAAATTTATTTTCATTGCAATAGTGTGTTGGAATTTTTTGTGTCTCTCACTCGGAA
GGACATATGGGAGGGCAAATCATTTAAAACATCAGAATGAGTATTTGGTTTAGAGTTTGGCAACATAT
GCCCATATGCTGGCTGCCATGAACAAAGGTTGGCTATAAAGAGGTCATCAGTATATGAAACAGCCCC
CTGCTGTCCATTCCTTATTCCATAGAAAAGCCTTGACTTGAGGTTAGATTTTTTTTATATTTTGTTTTGT
GTTATTTTTTTCTTTAACATCCCTAAAATTTTCCTTACATGTTTTACTAGCCAGATTTTTCCTCCTCTCCT
GACTACTCCCAGTCATAGCTGTCCCTCTTCTCTTATGGAGATCCCTCGACCTGCAGCCCAAGCTTGGC
GTAATCATGGTCATAGCTGTTTCCTGTGTGAAATTGTTATCCGCTCACAATTCCACACAACATACGAGC
CGGAAGCATAAAGTGTAAAGCCTGGGGTGCCTAATGAGTGAGCTAACTCACATTAATTGCGTTGCGC
TCACTGCCCGCTTTCCAGTCGGGAAACCTGTCGTGCCAGCGGATCCGCATCTCAATTAGTCAGCAACC
ATAGTCCCGCCCCTAACTCCGCCCATCCCGCCCCTAACTCCGCCCAGTTCCGCCCATTCTCCGCCCCA
TGGCTGACTAATTTTTTTTATTTATGCAGAGGCCGAGGCCGCCTCGGCCTCTGAGCTATTCCAGAAGT
AGTGAGGAGGCTTTTTTGGAGGCCTAGGCTTTTGCAAAAAGCTGTCGACTGCAGAGGCCTGCATGCA
AGCTTGGCGTAATCATGGTCATAGCTGTTTCCTGTGTGAAATTGTTATCCGCTCACAATTCCACACAAC
ATACGAGCCGGAAGCATAAAGTGTAAAGCCTGGGGTGCCTAATGAGTGAGCTAACTCACATTAATTG
CGTTGCGCTCACTGCCCGCTTTCCAGTCGGGAAACCTGTCGTGCCAGCTGCATTAATGAATCGGCCA
ACGCGCGGGGAGAGGCGGTTTGCGTATTGGGCGCTCTTCCGCTTCCTCGCTCACTGACTCGCTGCGC
TCGGTCGTTCGGCTGCGGCGAGCGGTATCAGCTCACTCAAAGGCGGTAATACGGTTATCCACAGAAT
CAGGGGATAACGCAGGAAAGAACATGTGAGCAAAAGGCCAGCAAAAGGCCAGGAACCGTAAAAAGG
CCGCGTTGCTGGCGTTTTTCCATAGGCTCCGCCCCCCTGACGAGCATCACAAAAATCGACGCTCAAGT
CAGAGGTGGCGAAACCCGACAGGACTATAAAGATACCAGGCGTTTCCCCCTGGAAGCTCCCTCGTGC
GCTCTCCTGTTCCGACCCTGCCGCTTACCGGATACCTGTCCGCCTTTCTCCCTTCGGGAAGCGTGGCG
CTTTCTCATAGCTCACGCTGTAGGTATCTCAGTTCGGTGTAGGTCGTTCGCTCCAAGCTGGGCTGTGT
GCACGAACCCCCCGTTCAGCCCGACCGCTGCGCCTTATCCGGTAACTATCGTCTTGAGTCCAACCCG
GTAAGACACGACTTATCGCCACTGGCAGCAGCCACTGGTAACAGGATTAGCAGAGCGAGGTATGTAG
GCGGTGCTACAGAGTTCTTGAAGTGGTGGCCTAACTACGGCTACACTAGAAGAACAGTATTTGGTATC
TGCGCTCTGCTGAAGCCAGTTACCTTCGGAAAAAGAGTTGGTAGCTCTTGATCCGGCAAACAAACCAC
CGCTGGTAGCGGTGGTTTTTTTGTTTGCAAGCAGCAGATTACGCGCAGAAAAAAAGGATCTCAAGAA
GATCCTTTGATCTTTTCTACGGGGTCTGACGCTCAGTGGAACGAAAACTCACGTTAAGGGATTTTGGT
CATGAGATTATCAAAAAGGATCTTCACCTAGATCCTTTTAAATTAAAAATGAAGTTTTAAATCAATCTAA
AGTATATATGAGTAAACTTGGTCTGACAGTTACCAATGCTTAATCAGTGAGGCACCTATCTCAGCGAT
CTGTCTATTTCGTTCATCCATAGTTGCCTGACTCCCCGTCGTGTAGATAACTACGATACGGGAGGGCT
TACCATCTGGCCCCAGTGCTGCAATGATACCGCGAGACCCACGCTCACCGGCTCCAGATTTATCAGC
AATAAACCAGCCAGCCGGAAGGGCCGAGCGCAGAAGTGGTCCTGCAACTTTATCCGCCTCCATCCAG
TCTATTAATTGTTGCCGGGAAGCTAGAGTAAGTAGTTCGCCAGTTAATAGTTTGCGCAACGTTGTTGC
CATTGCTACAGGCATCGTGGTGTCACGCTCGTCGTTTGGTATGGCTTCATTCAGCTCCGGTTCCCAAC
GATCAAGGCGAGTTACATGATCCCCCATGTTGTGCAAAAAAGCGGTTAGCTCCTTCGGTCCTCCGATC
GTTGTCAGAAGTAAGTTGGCCGCAGTGTTATCACTCATGGTTATGGCAGCACTGCATAATTCTCTTAC
TGTCATGCCATCCGTAAGATGCTTTTCTGTGACTGGTGAGTACTCAACCAAGTCATTCTGAGAATAGT
GTATGCGGCGACCGAGTTGCTCTTGCCCGGCGTCAATACGGGATAATACCGCGCCACATAGCAGAAC
TTTAAAAGTGCTCATCATTGGAAAACGTTCTTCGGGGCGAAAACTCTCAAGGATCTTACCGCTGTTGA
GATCCAGTTCGATGTAACCCACTCGTGCACCCAACTGATCTTCAGCATCTTTTACTTTCACCAGCGTTT
CTGGGTGAGCAAAAACAGGAAGGCAAAATGCCGCAAAAAAGGGAATAAGGGCGACACGGAAATGTT
GAATACTCATACTCTTCCTTTTTCAATATTATTGAAGCATTTATCAGGGTTATTGTCTCATGAGCGGATA
CATATTTGAATGTATTTAGAAAAATAAACAAATAGGGGTTCCGCGCACATTTCCCCGAAAAGTGCCAC
CTGACGTCTAAGAAACCATTATTATCATGACATTAACCTATAAAAATAGGCGTATCACGAGGCCCTTTC
GTCTCGCGCGTTTCGGTGATGACGGTGAAAACCTCTGACACATGCAGCTCCCGGAGACGGTCACAGC
TTGTCTGTAAGCGGATGCCGGGAGCAGACAAGCCCGTCAGGGCGCGTCAGCGGGTGTTGGCGGGTG
TCGGGGCTGGCTTAACTATGCGGCATCAGAGCAGATTGTACTGAGAGTGCACCATATGCGGTGTGAA
ATACCGCACAGATGCGTAAGGAGAAAATACCGCATCAGGCGCCATTCGCCATTCAGGCTGCGCAACT
GTTGGGAAGGGCGATCGGTGCGGGCCTCTTCGCTATTACGCCAGCTGGCGAAAGGGGGATGTGCTG
CAAGGCGATTAAGTTGGGTAACGCCAGGGTTTTCCCAGTCACGACGTTGTAAAACGACGGCCAGTGA
ATTC
pCalH12 TL20c rGbGM G3320A 7SKsh734 400 1AT (SEO ID NO: 114)
GGCCGCCTCGGCCAAACAGCCCTTGAGTTTACCACTCCCTATCAGTGATAGAGAAAAGTGAAAGTCG
AGTTTACCACTCCCTATCAGTGATAGAGAAAAGTGAAAGTCGAGTTTACCACTCCCTATCAGTGATAG
AGAAAAGTGAAAGTCGAGTTTACCACTCCCTATCAGTGATAGAGAAAAGTGAAAGTCGAGTTTACCAG
TCCCTATCAGTGATAGAGAAAAGTGAAAGTCGAGTTTACCACTCCCTATCAGTGATAGAGAAAAGTGA
AAGTCGAGTTTACCACTCCCTATCAGTGATAGAGAAAAGTGAAAGTCGAGCTCGCCATGGGAGGCGT
GGCCTGGGGGGACTGGGGAGTGGCGAGCCCTCAGATCCTGCATATAAGCAGCTGCTTTTTGCCTGT
ACTGGGTCTCTCTGGTTAGACCAGATCTGAGCCTGGGAGCTCTCTGGCTAACTAGGGAACCCACTGC
TTAAGCCTCAATAAAGCTTGCCTTGAGTGCTTCAAGTAGTGTGTGCCCGTCTGTTGTGTGACTCTGGT
AACTAGAGATCCCTCAGACCCTTTTAGTCAGTGTGGAAAATCTCTAGCAGTGGCGCCCGAACAGGGA
CTTGAAAGCGAAAGGGAAACCAGAGGAGCTCTCTCGACGCAGGACTCGGCTTGCTGAAGCGCGCAC
GGCAAGAGGCGAGGGGCGGCGACTGGTGAGTACGCCAAAAATTTTGACTAGCGGAGGCTAGAAGGA
GAGAGATGGGTGCGAGAGCGTCAGTATTAAGCGGGGGAGAATTAGATCGCGATGGGAAAAAATTCG
GTTAAGGCCAGGGGGAAAGAAAAAATATAAATTAAAACATATAGTATGGGCAAGCAGGGAGCTAGAA
CGATTCGCAGTTAATACTGGCCTGTTAGAAACATCAGAAGGCTGTAGACAAATACTGGGACAGCTACA
ACCATCCCTTCAGACAGGATCAGAAGAACTTAGATCATTATATAATACAGTAGCAACCCTCTATTGTGT
GCATCAAAGGATAGAGATAAAAGACACCAAGGAAGCTTTAGACAAGATAGAGGAAGAGCAAAACAAA
AGTAAGAAAAAAGCACAGCAAGCAGCAGGATCTTCAGACCTGGAAATTCCCTACAATCCCCAAAGTCA
AGGAGTAGTAGAATCTATGAATAAAGAATTAAAGAAAATTATAGGACAGGTAAGAGATCAGGCTGAAC
ATCTTAAGACAGCAGTACAAATGGCAGTATTCATCCACAATTTTAAAAGAAAAGGGGGGATTGGGGG
GTACAGTGCAGGGGAAAGAATAGTAGACATAATAGCAACAGACATACAAACTAAAGAATTACAAAAAC
AAATTACAAAAATTCAAAATTTTCGGGTTTATTACAGGGACAGCAGAAATCCACTTTGGAAAGGACCA
GCAAAGCTCCTCTGGAAAGGTGAAGGGGCAGTAGTAATACAAGATAATAGTGACATAAAAGTAGTGC
CAAGAAGAAAAGCAAAGATCATTAGGGATTATGGAAAACAGATGGCAGGTGATGATTGTGTGGCAAG
TAGACAGGATGAGGATTAGAACATGGAAAAGTTTAGTAAAACACCATAAGGAGGAGATATGAGGGAC
AATTGGAGAAGTGAATTATATAAATATAAAGTAGTAAAAATTGAACCATTAGGAGTAGCACCCACCAA
GGCAAAGAGAAGAGTGGTGCAGAGAGAAAAAAGAGCAGTGGGAATAGGAGCTTTGTTCCTTGGGTT
CTTGGGAGCAGCAGGAAGCACTATGGGCGCAGCGTCAATGACGCTGACGGTACAGGCCAGACAATT
ATTGTCTGGTATAGTGCAGCAGCAGAACAATTTGCTGAGGGCTATTGAGGCGCAACAGCATCTGTTG
CAACTCACAGTCTGGGGCATCAAGCAGCTCCAGGCAAGAATCCTGGCTGTGGAAAGATACCTAAAGG
ATCAACAGCTCCTGGGGATTTGGGGTTGCTCTGGAAAACTCATTTGCACCACTGCTGTGCCTTGGAAT
GCTAGTTGGAGTAATAAATCTCTGGAACAGATTTGGAATCACACGACCTGGATGGAGTGGGACAGAG
AAATTAACAATTACACAAGCTTAATACACTCCTTAATTGAAGAATCGCAAAACCAGCAAGAAAAGAATG
AACAAGAATTATTGGAATTAGATAAATGGGCAAGTTTGTGGAATTGGTTTAACATAACAAATTGGCTGT
GGTATATAAAATTATTCATAATGATAGTAGGAGGCTTGGTAGGTTTAAGAATAGTTTTTGCTGTACTTT
CTATAGTGAATAGAGTTAGGCAGGGATATTCACCATTATCGTTTCAGACCCACCTCCCAACCCCGAGG
GGACCGAGCTCAAGCTTCGAACGCGTGGGGATCCTCTAGAGTCGAGCTCGCGAGGATCATCACCGG
TGCTAGCCGGAGCCAGAAGCACCATAAGGGACATGATAAGGGAGCCAGCAGACCTCTGATCTCTTCC
TGAATGCTAATCTTAAACATCCTGAGGAAGAATGGGACTTCCATTTGGGGTGGGCCTATGATAGGGTA
ATAAGACAGTAGTGAATATCAAGCTACAAAAAGCCCCCTTTCAAATTCTTCTCAGTCCTAACTTTTCAT
ACTAAGCCCAGTCCTTCCAAAGCAGACTGTGAAAGAGTGATAGTTCCGGGAGACTAGCACCGGCTAG
CCGAGCTTGGAACACTTTCCCTTCATTAAGAACCATCCTTGCTACTCAGCTGCAATCAATCCAGCCCCC
AGGTCTTCACTGAACCTTTTCCCATCTCTTCCAAAACATCTGTTTCTGAGAAGTCCTGTCCTATAGAGG
TCTTTCTTCCCACCGGATTTCTCCTACACCATTTACTCCCACTTGCAGAACTCCCGTGTACAAGTGTCT
TTACTGCTTTTATTTGCTCAACAAAATGCACATCTCATATAAAAATAAATGAGGAGCATGCACACACCA
CAAACACAAACAGGCATGCAGAAATACACATACACACTTCCCTCAATATAAACCCTTTGTGGCTCATAT
ATTTAAAAAGATGTAAAAAAAAGAGCTGAAGAAAATCATGTGTGATCTCTCAGCAGAATAGATTTATTA
TTTGTATTGCTTGCAGAATAAAGCCTATCCTTGAAAGCTCTGAATCATGGGCAAGAGGCTCAGTGGTA
TCTGGAGGACAGGGCACTGGCCACTGCAGTCACCATCTTCTGCCAGGAAGCCTGCACCTCAGGGGTG
AATTCTTTGCCAAAGTGAATGGCCAGCACGGTGACCAGCACGTTGCCCAGGAGCTGTGGGAGGAAGA
TAAGAGATATGAACATGATTAGCAAAAGGGCCTAGCTTGGACTCAGAATAATCCAGCCTTATCCCAAC
CATAAAATAAAAGCAGAATGGTAGCTGGATTGTAGCTGCTATTAGCAATATGAAACCTCTTACATCAGT
TACAATTTATATGCAGAAATATTTATATGCAGAAATATTGCTATTGCCTTAACCCAGAAATTATCACTGT
TATTCTTTAGAATGGTGCAAAGAGGCATGATACATTGTATCATTATTGCCCTGAAAGAAAGAGATTAG
GGAAAGTATTAGAAATAAGATAAACAAAAAAGTATATTAAAAGAAGAAAGCATTTTTTAAAATTACAAA
TGCAAAATTACCCTGATTTGGTCAATATGTGTACCCTGTTACTTCTCCCCTTCCTATGACATGAACTTA
ACCATAGAAAAGAAGGGGAAAGAAAACATCAAGGGTCCCATAGACTCACCTTGAAGTTCTCAGGATC
CACATGCAGCTTGTCACAGTGCAGTTCACTCAGCTGGGCAAAGGTGCCCTTGAGATCATCCAGGTGC
TTTATGGCATCTCCCAAGGAAGTCAGCACCTTCTTGCCATGTGCCTTGACTTTGGGGTTGCCCATGAT
GGCAGAGGCAGAGGACAGGTTGCCAAAGCTGTCAAAGAACCTCTGGGTCCATGGGTAGACAACCAG
GAGCCTGTGAGATTGACAAGAACAGTTTGACAGTCAGAAGGTGCCACAAATCCTGAGAAGCAACCTG
GACTTTTGCCAGGCACAGGGTCCTTCCTTCCCTCCCTTGTCCTGGTCACCAGAGCCTACCTTCCCAGG
GTTTCTCCTCCAGCATCTTCCACATTCACCTTGTCCCACAGGCTTGTGATAGTAGCCTTGTCCTCCTCT
GTGAAATGACCCATGGTGTCTGTTTGAGGTTGCTAGTGAACACAGTTGTGTCAGAAGCAAATGTAAGC
AATAGATGGCTCTGCCCTGACTTTTATGCCCAGCCCTGGCTCCTGCCCTCCCTGCTCCTGGGAGTAGA
TTGGCCAACCCTAGGGTGTGGCTCCACAGGGTGAGGTCTAAGTGATGACAGCCGTACCTGTCCTTGG
CTCTTCTGGCACTGGCTTAGGAGTTGGACTTCAAACCCTCAGCCCTCCCTCTAAGATATATCTCTTGG
CCCCATACCATCAGTACAAATTGCTACTAAAAACATCCTCCTTTGCAAGTGTATTTACGACGGTATCGA
TGTATGTGAGCATGTGTCCTCTAACAGCACAGGCCTTTTGCCACCTAGCTGTCCAGGGGTGCCTTAAA
ATGGCAAACAAGGTTTGTTTTCTTTTCCTGTTTTCATGCCTTCCTCTTCCATATCCTTGTTTCATATTAAT
ACATGTGTATAGATCCTAAAAATCTATACACATGTATTAATAAAGCCTGATTCTGCCGCTTCTAGGTAT
AGAGGCCACCTGCAAGATAAATATTTGATTCACAATAACTAATCATTCTATGGCAATTGATAACAACAA
ATATATATATATATATATATATACGTATATGTGTATATATATATATATATTCAGGAAATAATATATTCTAG
AATATGTCACATTCTGTCTCAGGCATCCATTTTCTTTATGATGCCGTTTGAGGTGGAGTTTTAGTCAGG
TGGTCAGCTTCTCCTTTTTTTTGCCATCTGCCCTGTAAGCATCCTGCTGGGGACCCAGATAGGAGTCA
TCACTCTAGGCTGAGAACATCTGGGCACACACCCTAAGCCTCAGCATGACTCATCATGACTCAGCATT
GCTGTGCTTGAGCCAGAAGGTTTGCTTAGAAGGTTACACAGAACCAGAAGGCGGGGGTGGGGCACT
GACCCCGACAGGGGCCTGGCCAGAACTGCTCATGCTTGGACTATGGGAGGTCACTAATGGAGACAC
ACAGAAATGTAACAGGAACTAAGGGAATTCCGGTGCCCTGCTTAGGAGCTTAATCTTTAATGAAAGCT
AAGCTTTCATTAAAAAAAGTCTAACCAGCTGCATTCGACTTTGACTGCAGCAGCTGGTTAGAAGGTTC
TACTGGAGGAGGGTCCCAGCCCATTGCTAAATTAACATCAGGCTCTGAGACTGGCAGTATATCTCTAA
CAGTGGTTGATGCTATCTTCTGGAACTTGCCTGCTACATTGAGACCACTGACCCATACATAGGAAGCC
CATAGCTCTGTCCTGAACTGTTAGGCCACTGGTCCAGAGAGTGTGCATCTCCTTTGATCCTCATAATA
ACCCTATGAGATAGACACAATTATTACTCTTACTTTATAGATGATGATCCTGAAAACATAGGAGTCAAG
GCACTTGCCCCTAGCTGGGGGTATAGGGGAGCAGTCCCATGTAGTAGTAGAATGAAAAATGCTGCTA
TGCTGTGCCTCCCCCACCTTTCCCATGTCTGCCCTCTACTCATGGTCTATCTCTCCTGGCTCCTGGGA
GTCATGGACTCCACCCAGCACCACCAACCTGACCTAACCACCTATCTGAGCCTGCCAGCCTATAACCC
ATCTGGGCCCTGATAGCTGGTGGCCAGCCCTGACCCCACCCCACCCTCCCTGGAACCTCTGATAGAC
ACATCTGGCACACCAGCTCGCAAAGTCACCGTGAGGGTCTTGTGTTTGCTGAGTCAAAATTCCTTGAA
ATCCAAGTCCTTAGAGACTCCTGCTCCCAAATTTACAGTCATAGACTTCTTCATGGCTGTCTCCTTTAT
CCACAGAATGATTCCTTTGCTTCATTGCCCCATCCATCTGATCCTCCTCATCAGTGCAGCACAGGGCC
CATGAGCAGTAGCTGCAGAGTCTCACATAGGTCTGGCACTGCCTCTGACATGTCCGACCTTAGGCAA
ATGCTTGACTCTTCTGAGCTCGGATCCCTTGAGCTCAGGAGGTCAAGGCTGCAGTGAGACATGATCTT
GCCACTGCACTCCAGCCTGGACAGCAGAGTGAAACCTTGCCTCACGAAACAGAATACAAAAACAAAC
AAACAAAAAACTGCTCCGCAATGCGCTTCCTTGATGCTCTACCACATAGGTCTGGGTACTTTGTACAC
ATTATCTCATTGCTGTTCATAATTGTTAGATTAATTTTGTAATATTGATATTATTCCTAGAAAGCTGAGG
CCTCAAGATGATAACTTTTATTTTCTGGACTTGTAATAGCTTTCTCTTGTATTCACCATGTTGTAACTTT
CTTAGAGTAGTAACAATATAAAGTTATTGTGAGTTTTTGCAAACACAGCAAACACAACGACCCATATAG
ACATTGATGTGAAATTGTCTATTGTCAATTTATGGGAAAACAAGTATGTACTTTTTCTACTAAGCCATT
GAAACAGGAATAACAGAACAAGATTGAAAGAATACATTTTCCGAAATTACTTGAGTATTATACAAAGAC
AAGCACGTGGACCTGGGAGGAGGGTTATTGTCCATGACTGGTGTGTGGAGACAAATGCAGGTTTATA
ATAGATGGGATGGCATCTAGCGCAATGACTTTGCCATCACTTTTAGAGAGCTCTTGGGGGCCCCAGTA
CACAAGAGGGGACGCAGGGTATATGTAGACATCTCATTCTTTTTCTTAGTGTGAGAATAAGAATAGCC
ATGACCTGAGTTTATAGACAATGAGCCCTTTTCTCTCTCCCACTCAGCAGCTATGAGATGGCTTGCCCT
GCCTCTCTACTAGGCTGACTCACTCCAAGGCCCAGCAATGGGCAGGGCTCTGTCAGGGCTTTGATAG
CACTATCTGCAGAGCCAGGGCCGAGAAGGGGTGGACTCCAGAGACTCTCCCTCCCATTCCCGAGCAG
GGTTTGCTTATTTATGCATTTAAATGATATATTTATTTTAAAAGAAATAACAGGAGACTGCCCAGCCCT
GGCTGTGACATGGAAACTATGTAGAATATTTTGGGTTCCATTTTTTTTTCCTTCTTTCAGTTAGAGGAA
AAGGGGCTCACTGCACATACACTAGACAGAAAGTCAGGAGCTTTGAATCCAAGCCTGATCATTTCCAT
GTCATACTGAGAAAGTCCCCACCCTTCTCTGAGCCTCAGTTTCTCTTTTTATAAGTAGGAGTCTGGAGT
AAATGATTTCCAATGGCTCTCATTTCAATACAAAATTTCCGTTTATTAAATGCATGAGCTTCCGTTACTC
CAAGACTGAGAAGGAAATTGAACCTGAGACTCATTGACTGGCAAGATGTCCCCAGAGGCTCTCATTCA
GCAATAAAATTCTCACCTTCACCCAGGCCCACTAGTGTCAGATTTGCATGCGTTCGCGTATCGACGTG
CAGTATTTAGCATGCCCCACCCATCTGCAAGGCATTCTGGATAGTGTCAAAACAGCCGGAAATCAAGT
CCGTTTATCTCAAACTTTAGCATTTTGGGAATAAATGATATTTGCTATGCTGGTTAAATTAGATTTTAGT
TAAATTTCCTGCTGAAGCTCTAGTACGATAAGTAACTTGACCTAAGTGTAAAGTTGAGATTTCCTTCAG
GTTTATATAGCTTGTGCGCCGCCTGGGTACCTCAGGATATGCCCTTGACTATTTGTCCGACATAGTCA
AGGGCATATCCTTTTTTGTGCGGCCGCATCGATGCCGTAGTACCTTTAAGACCAATGACTTACAAGGC
AGCTGTAGATCTTAGCCACTTTTTAAAAGAAAAGGGGGGACTGGAAGGGCTAATTCACTCCCAAAGAA
GACAAGATCCCTGCAGGCATTCAAGGCCAGGCTGGATGTGGCTCTGGGCAGCCTGGGCTGCTGGTT
GATGACCCTGCACATAGCAGGGGGTTGGATCTGGATGAGCACTGTGCTCCTTTGCAACCCAGGCCGT
TCTATGATTCTGTCATTCTAAATCTCTCTTTCAGCCTAAAGCTTTTTCCCCGTATCCCCCCIGGTGTCTG
CAGGCTCAAAGAGCAGCGAGAAGCGTTCAGAGGAAAGCGATCCCGTGCCACCTTCCCCGTGCCCGG
GCTGTCCCCGCACGCTGCCGGCTCGGGGATGCGGGGGGAGCGCCGGACCGGAGCGGAGCCCCGG
GCGGCTCGCTGCTGCCCCCTAGCGGGGGAGGGACGTAATTACATCCCTGGGGGCTTTGGGGGGGG
GCTGTCCCCGTGAGCTCCCCAGATCTGCTTTTTGCCTGTACTGGGTCTCTCTGGTTAGACCAGATCTG
AGCCTGGGAGCTCTCTGGCTAACTAGGGAACCCACTGCTTAAGCCTCAATAAAGCTTCAGCTGCTCGA
GCTAGCAGATCTTTTTCCCTCTGCCAAAAATTATGGGGACATCATGAAGCCCCTTGAGCATCTGACTT
CTGGCTAATAAAGGAAATTTATTTTCATTGCAATAGTGTGTTGGAATTTTTTGTGTCTCTCACTCGGAA
GGACATATGGGAGGGCAAATCATTTAAAACATCAGAATGAGTATTTGGTTTAGAGTTTGGCAACATAT
GCCCATATGCTGGCTGCCATGAACAAAGGTTGGCTATAAAGAGGTCATCAGTATATGAAACAGCCCC
CTGCTGTCCATTCCTTATTCCATAGAAAAGCCTTGACTTGAGGTTAGATTTTTTTTATATTTTGTTTTGT
GTTATTTTTTTCTTTAACATCCCTAAAATTTTCCTTACATGTTTTACTAGCCAGATTTTTCCTCCTCTCCT
GACTACTCCCAGTCATAGCTGTCCCTCTTCTCTTATGGAGATCCCTCGACCTGCAGCCCAAGCTTGGC
GTAATCATGGTCATAGCTGTTTCCTGTGTGAAATTGTTATCCGCTCACAATTCCACACAACATACGAGC
CGGAAGCATAAAGTGTAAAGCCTGGGGTGCCTAATGAGTGAGCTAACTCACATTAATTGCGTTGCGC
TCACTGCCCGCTTTCCAGTCGGGAAACCTGTCGTGCCAGCGGATCCGCATCTCAATTAGTCAGCAACC
ATAGTCCCGCCCCTAACTCCGCCCATCCCGCCCCTAACTCCGCCCAGTTCCGCCCATTCTCCGCCCCA
TGGCTGACTAATTTTTTTTATTTATGCAGAGGCCGAGGCCGCCTCGGCCTCTGAGCTATTCCAGAAGT
AGTGAGGAGGCTTTTTTGGAGGCCTAGGCTTTTGCAAAAAGCTGTCGACTGCAGAGGCCTGCATGCA
AGCTTGGCGTAATCATGGTCATAGCTGTTTCCTGTGTGAAATTGTTATCCGCTCACAATTCCACACAAC
ATACGAGCCGGAAGCATAAAGTGTAAAGCCTGGGGTGCCTAATGAGTGAGCTAACTCACATTAATTG
CGTTGCGCTCACTGCCCGCTTTCCAGTCGGGAAACCTGTCGTGCCAGCTGCATTAATGAATCGGCCA
ACGCGCGGGGAGAGGCGGTTTGCGTATTGGGCGCTCTTCCGCTTCCTCGCTCACTGACTCGCTGCGC
TCGGTCGTTCGGCTGCGGCGAGCGGTATCAGCTCACTCAAAGGCGGTAATACGGTTATCCACAGAAT
CAGGGGATAACGCAGGAAAGAACATGTGAGCAAAAGGCCAGCAAAAGGCCAGGAACCGTAAAAAGG
CCGCGTTGCTGGCGTTTTTCCATAGGCTCCGCCCCCCTGACGAGCATCACAAAAATCGACGCTCAAGT
CAGAGGTGGCGAAACCCGACAGGACTATAAAGATACCAGGCGTTTCCCCCTGGAAGCTCCCTCGTGC
GCTCTCCTGTTCCGACCCTGCCGCTTACCGGATACCTGTCCGCCTTTCTCCCTTCGGGAAGCGTGGCG
CTTTCTCATAGCTCACGCTGTAGGTATCTCAGTTCGGTGTAGGTCGTTCGCTCCAAGCTGGGCTGTGT
GCACGAACCCCCCGTTCAGCCCGACCGCTGCGCCTTATCCGGTAACTATCGTCTTGAGTCCAACCCG
GTAAGACACGACTTATCGCCACTGGCAGCAGCCACTGGTAACAGGATTAGCAGAGCGAGGTATGTAG
GCGGTGCTACAGAGTTCTTGAAGTGGTGGCCTAACTACGGCTACACTAGAAGAACAGTATTTGGTATC
TGCGCTCTGCTGAAGCCAGTTACCTTCGGAAAAAGAGTTGGTAGCTCTTGATCCGGCAAACAAACCAC
CGCTGGTAGCGGTGGTTTTTTTGTTTGCAAGCAGCAGATTACGCGCAGAAAAAAAGGATCTCAAGAA
GATCCTTTGATCTTTTCTACGGGGTCTGACGCTCAGTGGAACGAAAACTCACGTTAAGGGATTTTGGT
CATGAGATTATCAAAAAGGATCTTCACCTAGATCCTTTTAAATTAAAAATGAAGTTTTAAATCAATCTAA
AGTATATATGAGTAAACTTGGTCTGACAGTTACCAATGCTTAATCAGTGAGGCACCTATCTCAGCGAT
CTGTCTATTTCGTTCATCCATAGTTGCCTGACTCCCCGTCGTGTAGATAACTACGATACGGGAGGGCT
TACCATCTGGCCCCAGTGCTGCAATGATACCGCGAGACCCACGCTCACCGGCTCCAGATTTATCAGC
AATAAACCAGCCAGCCGGAAGGGCCGAGCGCAGAAGTGGTCCTGCAACTTTATCCGCCTCCATCCAG
TCTATTAATTGTTGCCGGGAAGCTAGAGTAAGTAGTTCGCCAGTTAATAGTTTGCGCAACGTTGTTGC
CATTGCTACAGGCATCGTGGTGTCACGCTCGTCGTTTGGTATGGCTTCATTCAGCTCCGGTTCCCAAC
GATCAAGGCGAGTTACATGATCCCCCATGTTGTGCAAAAAAGCGGTTAGCTCCTTCGGTCCTCCGATC
GTTGTCAGAAGTAAGTTGGCCGCAGTGTTATCACTCATGGTTATGGCAGCACTGCATAATTCTCTTAC
TGTCATGCCATCCGTAAGATGCTTTTCTGTGACTGGTGAGTACTCAACCAAGTCATTCTGAGAATAGT
GTATGCGGCGACCGAGTTGCTCTTGCCCGGCGTCAATACGGGATAATACCGCGCCACATAGCAGAAC
TTTAAAAGTGCTCATCATTGGAAAACGTTCTTCGGGGCGAAAACTCTCAAGGATCTTACCGCTGTTGA
GATCCAGTTCGATGTAACCCACTCGTGCACCCAACTGATCTTCAGCATCTTTTACTTTCACCAGCGTTT
CTGGGTGAGCAAAAACAGGAAGGCAAAATGCCGCAAAAAAGGGAATAAGGGCGACACGGAAATGTT
GAATACTCATACTCTTCCTTTTTCAATATTATTGAAGCATTTATCAGGGTTATTGTCTCATGAGCGGATA
CATATTTGAATGTATTTAGAAAAATAAACAAATAGGGGTTCCGCGCACATTTCCCCGAAAAGTGCCAC
CTGACGTCTAAGAAACCATTATTATCATGACATTAACCTATAAAAATAGGCGTATCACGAGGCCCTTTC
GTCTCGCGCGTTTCGGTGATGACGGTGAAAACCTCTGACACATGCAGCTCCCGGAGACGGTCACAGC
TTGTCTGTAAGCGGATGCCGGGAGCAGACAAGCCCGTCAGGGCGCGTCAGCGGGTGTTGGCGGGTG
TCGGGGCTGGCTTAACTATGCGGCATCAGAGCAGATTGTACTGAGAGTGCACCATATGCGGTGTGAA
ATACCGCACAGATGCGTAAGGAGAAAATACCGCATCAGGCGCCATTCGCCATTCAGGCTGCGCAACT
GTTGGGAAGGGCGATCGGTGCGGGCCTCTTCGCTATTACGCCAGCTGGCGAAAGGGGGATGTGCTG
CAAGGCGATTAAGTTGGGTAACGCCAGGGTTTTCCCAGTCACGACGTTGTAAAACGACGGCCAGTGA
ATTC
β-globin promoter (SEQ ID NO: 115)
GGTGTCTGTTTGAGGTTGCTAGTGAACACAGTTGTGTCAGAAGCAAATGTAAGCAATAGATGGCTCTG
CCCTGACTTTTATGCC
β-globin promoter (SEQ ID NO: 116)
AAGCAATAGATGGCTCTGCCCTGACTTTTATGCCCAGCCCTGGCTCCTGCCCTCCCTGCTCCTGGGAG
TAGATTGGCCAACCCTAGGGTGTGGCTCCACAGGGTGAGGTCTAAGTGATGACAGCCGTACCTGTCC
TTGGCTCTTCTGGCACTGGCTTAGGAGTTGGACTTCAAACCCTCAGCCCTCCCTCTAAGATATATCTCT
TGGCCCCATACCATCAGTACAAATTGCTACTAAAAACATCCTCCTTTGCAAGTGTATTTAC
β-globin promoter (SEQ ID NO: 117)
GGTGTCTGTTTGAGGTTGCTAGTGAACACAGTTGTGTCAGAAGCAAATGTAAGCAATAGATGGCTCTG
CCCTGACTTTTATGCCCAGCCCTGGCTCCTGCCCTCCCTGCTCCTGGGAGTAGATTGGCCAACCCTAG
GGTGTGGCTCCACAGGGTGAGGTCTAAGTGATGACAGCCGTACCTGTCCTTGGCTCTTCTGGCACTG
GCTTAGGAGTTGGACTTCAAACCCTCAGCCCTCCCTCTAAGATATATCTCTTGGCCCCATACCATCAGT
ACAAATTGCTACTAAAAACATCCTCCTTTGCAAGTGTATTTACGA
Unmodified γ-globin transgene (SEQ ID NO: 118)
ATGGGTCATTTCACAGAGGAGGACAAGGCTACTATCACAAGCCTGTGGGACAAGGTGAATGTGGAAG
ATGCTGGAGGAGAAACCCTGGGAAGgtaggctctggtgaccaggacaagggagggaaggaaggaccctgtgcctgg
caaaagtccaggttgcttctcaggatttgtggcaccttctgactgtcaaactgttcttgtcaatctcacaGGCTCCTGGTTGTCTA
CCCATGGACCCAGAGGTTCTTTGACAGCTTTGGCAACCTGTCCTCTGCCTCTGCCATCATGGGCAACC
CCAAAGTCAAGGCACATGGCAAGAAGGTGCTGACTTCCTTGGGAGATGCCATAAAGCACCTGGATGA
TCTCAAGGGCACCTTTGCCCAGCTGAGTGAACTGCACTGTGACAAGCTGCATGTGGATCCTGAGAAC
TTCAAGgtgagtctatgggacccttgatgttttctttccccttcttttctatggttaagttcatgtcataggaaggggagaagtaacag
ggtacacatattgaccaaatcagggtaattttgcatttgtaattttaaaaaatgctttcttcttttaatatacttttttgtttatcttatttc
taatactttccctaatctctttctttcagggcaataatgatacaatgtatcatgcctctttgcaccattctaaagaataacagtgataatttct
gggttaaggcaatagcaatatttctgcatataaatatttctgcatataaattgtaactgatgtaagaggtttcatattgctaatagcagc
tacaatccagctaccattctgcttttattttatggttgggataaggctggattattctgagtccaagctaggcccttttgctaatcatgttc
atacctcttatcttcctcccacagCTCCTGGGCAACGTGCTGGTCACCGTGCTGGCCATTCACTTTGGCAAAGA
ATTCACCCCTGAGGTGCAGGCTTCCTGGCAGAAGATGGTGACTGCAGTGGCCAGTGCCCTGTCCTCC
AGATACCACTGAG
Unmodified γ-globin transgene-reverse complement (exons in uppercase) (SEQ ID NO: 119)
CTCAGTGGTATCTGGAGGACAGGGCACTGGCCACTGCAGTCACCATCTTCTGCCAGGAAGCCTGCAC
CTCAGGGGTGAATTCTTTGCCAAAGTGAATGGCCAGCACGGTGACCAGCACGTTGCCCAGGAGctgtg
ggaggaagataagaggtatgaacatgattagcaaaagggcctagcttggactcagaataatccagccttatcccaaccataaaat
aaaagcagaatggtagctggattgtagctgctattagcaatatgaaacctcttacatcagttacaatttatatgcagaaatatttatat
gcagaaatattgctattgccttaacccagaaattatcactgttattctttagaatggtgcaaagaggcatgatacattgtatcattattg
ccctgaaagaaagagattagggaaagtattagaaataagataaacaaaaaagtatattaaaagaagaaagcattttttaaaatta
caaatgcaaaattaccctgatttggtcaatatgtgtaccctgttacttctccccttcctatgacatgaacttaaccatagaaaagaagg
ggaaagaaaacatcaagggtcccatagactcacCTTGAAGTTCTCAGGATCCACATGCAGCTTGTCACAGTGCA
GTTCACTCAGCTGGGCAAAGGTGCCCTTGAGATCATCCAGGTGCTTTATGGCATCTCCCAAGGAAGT
CAGCACCTTCTTGCCATGTGCCTTGACTTTGGGGTTGCCCATGATGGCAGAGGCAGAGGACAGGTTG
CCAAAGCTGTCAAAGAACCTCTGGGTCCATGGGTAGACAACCAGGAGCctgtgagattgacaagaacagtttg
acagtcagaaggtgccacaaatcctgagaagcaacctggacttttgccaggcacagggtccttccttccctcccttgtcctggtcacc
agagcctacCTTCCCAGGGTTTCTCCTCCAGCATCTTCCACATTCACCTTGTCCCACAGGCTTGTGATAG
TAGCCTTGTCCTCCTCTGTGAAATGACCCAT
Unmodified γ-globin transgene-reverse complement-G to A mutation at SD1 (mutation in
bold and underlined) (SEQ ID NO: 120)
CTCAGTGGTATCTGGAGGACAGGGCACTGGCCACTGCAGTCACCATCTTCTGCCAGGAAGCCTGCAC
CTCAGGGGTGAATTCTTTGCCAAAGTGAATGGCCAGCACGGTGACCAGCACGTTGCCCAGGAGctgtg
ggaggaagataagagatatgaacatgattagcaaaagggcctagcttggactcagaataatccagccttatcccaaccataaaat
aaaagcagaatggtagctggattgtagctgctattagcaatatgaaacctcttacatcagttacaatttatatgcagaaatatttatat
gcagaaatattgctattgccttaacccagaaattatcactgttattctttagaatggtgcaaagaggcatgatacattgtatcattattg
ccctgaaagaaagagattagggaaagtattagaaataagataaacaaaaaagtatattaaaagaagaaagcattttttaaaatta
caaatgcaaaattaccctgatttggtcaatatgtgtaccctgttacttctccccttcctatgacatgaacttaaccatagaaaagaagg
ggaaagaaaacatcaagggtcccatagactcacCTTGAAGTTCTCAGGATCCACATGCAGCTTGTCACAGTGCA
GTTCACTCAGCTGGGCAAAGGTGCCCTTGAGATCATCCAGGTGCTTTATGGCATCTCCCAAGGAAGT
CAGCACCTTCTTGCCATGTGCCTTGACTTTGGGGTTGCCCATGATGGCAGAGGCAGAGGACAGGTTG
CCAAAGCTGTCAAAGAACCTCTGGGTCCATGGGTAGACAACCAGGAGCctgtgagattgacaagaacagtttg
acagtcagaaggtgccacaaatcctgagaagcaacctggacttttgccaggcacagggtccttccttccctcccttgtcctggtcacc
agagcctacCTTCCCAGGGTTTCTCCTCCAGCATCTTCCACATTCACCTTGTCCCACAGGCTTGTGATAG
TAGCCTTGTCCTCCTCTGTGAAATGACCCAT
Unmodified γ-globin transgene + polyA signal (SEQ ID NO: 121)
ATGGGTCATTTCACAGAGGAGGACAAGGCTACTATCACAAGCCTGTGGGACAAGGTGAATGTGGAAG
ATGCTGGAGGAGAAACCCTGGGAAGgtaggctctggtgaccaggacaagggagggaaggaaggaccctgtgcctgg
caaaagtccaggttgcttctcaggatttgtggcaccttctgactgtcaaactgttcttgtcaatctcacaGGCTCCTGGTTGTCTA
CCCATGGACCCAGAGGTTCTTTGACAGCTTTGGCAACCTGTCCTCTGCCTCTGCCATCATGGGCAACC
CCAAAGTCAAGGCACATGGCAAGAAGGTGCTGACTTCCTTGGGAGATGCCATAAAGCACCTGGATGA
TCTCAAGGGCACCTTTGCCCAGCTGAGTGAACTGCACTGTGACAAGCTGCATGTGGATCCTGAGAAC
TTCAAGgtgagtctatgggacccttgatgttttctttccccttcttttctatggttaagttcatgtcataggaaggggagaagtaacag
ggtacacatattgaccaaatcagggtaattttgcatttgtaattttaaaaaatgctttcttcttttaatatacttttttgtttatcttatttc
taatactttccctaatctctttctttcagggcaataatgatacaatgtatcatgcctctttgcaccattctaaagaataacagtgataatttct
gggttaaggcaatagcaatatttctgcatataaatatttctgcatataaattgtaactgatgtaagaggtttcatattgctaatagcagc
tacaatccagctaccattctgcttttattttatggttgggataaggctggattattctgagtccaagctaggcccttttgctaatcatgttc
atacctcttatcttcctcccacagCTCCTGGGCAACGTGCTGGTCACCGTGCTGGCCATTCACTTTGGCAAAGA
ATTCACCCCTGAGGTGCAGGCTTCCTGGCAGAAGATGGTGACTGCAGTGGCCAGTGCCCTGTCCTCC
AGATACCACTGAGcctcttgcccatgattcagagctttcaaggataggctttattctgcaagcaatacaaataataaatctattct
gctgagagatcacacatgattttcttcagctcttttttttacatctttttaaatatatgagccacaaagggtttatattgagggaagtgtgt
atgtgtatttctgcatgcctgtttgtgtttgtggtgtgtgcatgctcctcatttatttttatatgagatgtgcattttgttgagcaaataaaa
gcagtaaagacacttgtacacgggagttctgcaagtgggagtaaatggtgtaggagaaatccggtgggaagaaagacctctatag
gacaggacttctcagaaacagatgttttggaagagatgggaaaaggttcagtgaagacctgggggctggattgattgcagctgag
tagcaaggatggttcttaatgaagggaaagtgttccaagctcggctagccggtgctagtctcccggaactatcactctttcacagtct
gctttggaaggactgggcttagtatgaaaagttaggactgagaagaatttgaaagggggctttttgtagcttgatattcactactgtc
ttattaccctatcataggcccaccccaaatggaagtcccattcttcctcaggatgtttaagattagcattcaggaagagatcagaggtc
tgctggctcccttatcatgtcccttatggtgcttctggctccggctagcaccggtgatgatcctcgcgagctcgactctagaggatcccc
Unmodified γ-globin transgene + polyA signal-reverse complement (SEQ ID NO: 122)
ggggatcctctagagtcgagctcgcgaggatcatcaccggtgctagccggagccagaagcaccataagggacatgataagggag
ccagcagacctctgatctcttcctgaatgctaatcttaaacatcctgaggaagaatgggacttccatttggggtgggcctatgatagg
gtaataagacagtagtgaatatcaagctacaaaaagccccctttcaaattcttctcagtcctaacttttcatactaagcccagtccttcc
aaagcagactgtgaaagagtgatagttccgggagactagcaccggctagccgagcttggaacactttcccttcattaagaaccatc
cttgctactcagctgcaatcaatccagcccccaggtcttcactgaaccttttcccatctcttccaaaacatctgtttctgagaagtcctgtc
ctatagaggtctttcttcccaccggatttctcctacaccatttactcccacttgcagaactcccgtgtacaagtgtctttactgcttttattt
gctcaacaaaatgcacatctcatataaaaataaatgaggagcatgcacacaccacaaacacaaacaggcatgcagaaatacacat
acacacttccctcaatataaaccctttgtggctcatatatttaaaaagatgtaaaaaaaagagctgaagaaaatcatgtgtgatctctc
agcagaatagatttattatttgtattgcttgcagaataaagcctatccttgaaagctctgaatcatgggcaagaggCTCAGTGGT
ATCTGGAGGACAGGGCACTGGCCACTGCAGTCACCATCTTCTGCCAGGAAGCCTGCACCTCAGGGGT
GAATTCTTTGCCAAAGTGAATGGCCAGCACGGTGACCAGCACGTTGCCCAGGAGctgtgggaggaagata
agaggtatgaacatgattagcaaaagggcctagcttggactcagaataatccagccttatcccaaccataaaataaaagcagaatg
gtagctggattgtagctgctattagcaatatgaaacctcttacatcagttacaatttatatgcagaaatatttatatgcagaaatattgc
tattgccttaacccagaaattatcactgttattctttagaatggtgcaaagaggcatgatacattgtatcattattgccctgaaagaaag
agattagggaaagtattagaaataagataaacaaaaaagtatattaaaagaagaaagcattttttaaaattacaaatgcaaaatta
ccctgatttggtcaatatgtgtaccctgttacttctccccttcctatgacatgaacttaaccatagaaaagaaggggaaagaaaacatc
aagggtcccatagactcacCTTGAAGTTCTCAGGATCCACATGCAGCTTGTCACAGTGCAGTTCACTCAGCT
GGGCAAAGGTGCCCTTGAGATCATCCAGGTGCTTTATGGCATCTCCCAAGGAAGTCAGCACCTTCTT
GCCATGTGCCTTGACTTTGGGGTTGCCCATGATGGCAGAGGCAGAGGACAGGTTGCCAAAGCTGTCA
AAGAACCTCTGGGTCCATGGGTAGACAACCAGGAGCctgtgagattgacaagaacagtttgacagtcagaaggtg
ccacaaatcctgagaagcaacctggacttttgccaggcacagggtccttccttccctcccttgtcctggtcaccagagcctacCTTCC
CAGGGTTTCTCCTCCAGCATCTTCCACATTCACCTTGTCCCACAGGCTTGTGATAGTAGCCTTGTCCTC
CTCTGTGAAATGACCCAT
Unmodified γ-globin transgene + polyA signal- reverse complement-G to A mutation at
SD1 (mutation in bold and underlined) (SEQ ID NO: 123)
ggggatcctctagagtcgagctcgcgaggatcatcaccggtgctagccggagccagaagcaccataagggacatgataagggag
ccagcagacctctgatctcttcctgaatgctaatcttaaacatcctgaggaagaatgggacttccatttggggtgggcctatgatagg
gtaataagacagtagtgaatatcaagctacaaaaagccccctttcaaattcttctcagtcctaacttttcatactaagcccagtccttcc
aaagcagactgtgaaagagtgatagttccgggagactagcaccggctagccgagcttggaacactttcccttcattaagaaccatc
cttgctactcagctgcaatcaatccagcccccaggtcttcactgaaccttttcccatctcttccaaaacatctgtttctgagaagtcctgtc
ctatagaggtctttcttcccaccggatttctcctacaccatttactcccacttgcagaactcccgtgtacaagtgtctttactgcttttattt
gctcaacaaaatgcacatctcatataaaaataaatgaggagcatgcacacaccacaaacacaaacaggcatgcagaaatacacat
acacacttccctcaatataaaccctttgtggctcatatatttaaaaagatgtaaaaaaaagagctgaagaaaatcatgtgtgatctctc
agcagaatagatttattatttgtattgcttgcagaataaagcctatccttgaaagctctgaatcatgggcaagaggCTCAGTGGT
ATCTGGAGGACAGGGCACTGGCCACTGCAGTCACCATCTTCTGCCAGGAAGCCTGCACCTCAGGGGT
GAATTCTTTGCCAAAGTGAATGGCCAGCACGGTGACCAGCACGTTGCCCAGGAGctgtgggaggaagata
agagatatgaacatgattagcaaaagggcctagcttggactcagaataatccagccttatcccaaccataaaataaaagcagaatg
gtagctggattgtagctgctattagcaatatgaaacctcttacatcagttacaatttatatgcagaaatatttatatgcagaaatattgc
tattgccttaacccagaaattatcactgttattctttagaatggtgcaaagaggcatgatacattgtatcattattgccctgaaagaaag
agattagggaaagtattagaaataagataaacaaaaaagtatattaaaagaagaaagcattttttaaaattacaaatgcaaaatta
ccctgatttggtcaatatgtgtaccctgttacttctccccttcctatgacatgaacttaaccatagaaaagaaggggaaagaaaacatc
aagggtcccatagactcacCTTGAAGTTCTCAGGATCCACATGCAGCTTGTCACAGTGCAGTTCACTCAGCT
GGGCAAAGGTGCCCTTGAGATCATCCAGGTGCTTTATGGCATCTCCCAAGGAAGTCAGCACCTTCTT
GCCATGTGCCTTGACTTTGGGGTTGCCCATGATGGCAGAGGCAGAGGACAGGTTGCCAAAGCTGTCA
AAGAACCTCTGGGTCCATGGGTAGACAACCAGGAGCctgtgagattgacaagaacagtttgacagtcagaaggtg
ccacaaatcctgagaagcaacctggacttttgccaggcacagggtccttccttccctcccttgtcctggtcaccagagcctacCTTCC
CAGGGTTTCTCCTCCAGCATCTTCCACATTCACCTTGTCCCACAGGCTTGTGATAGTAGCCTTGTCCTC
CTCTGTGAAATGACCCAT

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

What is claimed is:

1. A lentiviral vector, comprising:
a first promoter operably linked to a first nucleic acid sequence, wherein the first nucleic acid sequence encodes a Wiskott-Aldrich Syndrome protein; and
a modified HS4-650 insulator, wherein:
when present in the vector, the modified HS4-650 insulator comprises an inactivated splice acceptor site 1 (SA1) relative to an unmodified HS4-650 insulator, and wherein:
SA1 is present in an unmodified HS4-650 insulator at nucleotide positions 385-386 with numbering relative to SEQ ID NO:2, wherein SEQ ID NO:2 is the reverse, complement sequence of the unmodified HS4-650 insulator set forth in SEQ ID NO:1; and/or
SA1 comprises the sequence TTGCATCCAG\hat{ }ACACCATCAA (SEQ ID NO:60), where \hat{ } represents the splice position.

2. The lentiviral vector of claim 1, wherein the modified HS4-650 insulator comprises, relative to an unmodified HS4-650 insulator, a mutation that inactivates SA1.

3. The lentiviral vector of claim 2, wherein the mutation is a mutation of the A at position 384 and/or a mutation of the G at position 385, with numbering relative to SEQ ID NO:2.

4. (canceled)

5. The lentiviral vector of claim 1, wherein the modified HS4-650 insulator comprises the sequence set forth in any one of SEQ ID NOs:3, 12, 21, 30, 39 and 48.

6. The lentiviral vector of claim 2, wherein the modified HS4-650 insulator comprises a mutation that inactivates splice acceptor site 2 (SA2) relative to an unmodified HS4-650 insulator, wherein SA2 is present in an unmodified HS4-650 insulator at nucleotide positions 446-447, with numbering relative to SEQ ID NO:2.

7. The lentiviral vector of claim 6, wherein the mutation is a mutation of the A at position 445 and/or a mutation of the G at position 446, with numbering relative to SEQ ID NO:2.

8. (canceled)

9. The lentiviral vector of claim 6, wherein the reverse complement sequence of the modified HS4-650 insulator comprises the sequence set forth in any one of SEQ ID NOs:4, 13, 22, 31, 40 and 49.

10. The lentiviral vector of claim 2, wherein the modified HS4-650 insulator comprises a mutation that inactivates splice acceptor site 3 (SA3) relative to an unmodified HS4-650 insulator, wherein SA3 is present in an unmodified HS4-650 insulator at nucleotide positions 456-457, with numbering relative to SEQ ID NO:2.

11. The lentiviral vector of claim 10, wherein the mutation is a mutation of the A at position 455 and/or a mutation of the G at position 456 with numbering relative to SEQ ID NO:2.

12. (canceled)

13. The lentiviral vector of claim 10, wherein the reverse complement sequence of the modified HS4-650 insulator comprises the sequence set forth in SEQ ID NOs:5, 6, 14, 15, 23, 24, 32, 33, 41, 42, 50 and 51.

14. The lentiviral vector of claim 1, wherein the modified HS4-650 insulator is in the opposite orientation to the first nucleic acid sequence.

15. The lentiviral vector of claim 1, wherein the first nucleic acid is in the forward orientation and the modified HS4-650 insulator is in the reverse orientation within the lentiviral vector.

16. The lentiviral vector of claim 1, wherein the modified HS4-650 insulator is in the same orientation as the first nucleic acid sequence, thereby inactivating SA1.

17. The lentiviral vector of claim 16, wherein the first nucleic acid and the modified HS4-650 insulator are in the forward orientation within the lentiviral vector.

18. A lentiviral vector, comprising:
a first promoter operably linked to a first nucleic acid sequence, the first nucleic acid sequence encoding a Wiskott-Aldrich Syndrome protein; and
a modified HS4-650 insulator, wherein:
when present in the vector, the modified HS4-650 insulator comprises an inactivated splice acceptor site 2 (SA2) relative to an unmodified HS4-650 insulator, and wherein:
SA2 is present in an unmodified HS4-650 insulator at nucleotide positions 446-447, with numbering relative to SEQ ID NO:2, wherein SEQ ID NO:2 is the reverse, complement sequence of the unmodified HS4-650 insulator set forth in SEQ ID NO:1; and/or
SA2 comprises the sequence ATCCCCCCAG\hat{ }GTGTCTGCAG (SEQ ID NO:61), where \hat{ } represents the splice position.

19-34. (canceled)

35. A lentiviral vector, comprising:
a first promoter operably linked to a first nucleic acid sequence, the first nucleic acid sequence encoding a Wiskott-Aldrich Syndrome protein; and
a modified HS4-650 insulator, wherein:
when present in the vector, the modified HS4-650 insulator comprises an inactivated splice acceptor site 3 (SA3) relative to an unmodified HS4-650 insulator, and wherein:
SA3 is present in an unmodified HS4-650 insulator at nucleotide positions 456-457, with numbering relative to SEQ ID NO:2, wherein SEQ ID NO:2 is the reverse, complement sequence of the unmodified HS4-650 insulator set forth in SEQ ID NO:1; and/or
SA3 comprises the sequence GTGTCTGCAG\hat{ }GCTCAAAGAG (SEQ ID NO:62), where \hat{ } represents the splice position.

36-51. (canceled)

52. The lentiviral vector of claim 1, wherein the modified HS4-650 insulator is downstream of the first nucleic acid sequence.

53. The lentiviral vector of claim 1, wherein the Wiskott-Aldrich Syndrome protein comprises an amino acid sequence set forth in SEQ ID NO: 76 or a sequence having at least 95% sequence identity thereto.

54. (canceled)

55. The lentiviral vector of claim 1, further comprising a Woodchuck Hepatitis Virus (WHV) Posttranscriptional Regulatory Element (WPRE) between the first nucleic acid sequence and the modified HS4-650 insulator.

56. (canceled)

57. The lentiviral vector of claim 1, comprising a sequence selected from the group consisting of: the sequence set forth as nucleotides 3098-6006 of SEQ ID NO:57 or a sequence having at least 90%, 91%, 92%, 93%, 94%, 95%, 96%, 97%, 98% or 99% sequence identity thereto; the sequence set forth as nucleotides 3098-6009 of SEQ ID NO:58 or a sequence having at least 90%, 91%, 92%, 93%, 94%, 95%, 96%, 97%, 98% or 99% sequence identity thereto; and the sequence set forth as nucleotides 3098-6006 of SEQ ID NO:59 or a sequence having at least 90%, 91%, 92%, 93%, 94%, 95%, 96%, 97%, 98% or 99% sequence identity thereto.

58. The lentiviral vector of claim 1, wherein the first promoter is an MND promoter.

59. (canceled)

60. The lentiviral vector of claim 1, comprising a sequence selected from the group consisting of: the sequence set forth as nucleotides 2710-6006 of SEQ ID NO:57 or a sequence having at least 90%, 91%, 92%, 93%, 94%, 95%, 96%, 97%, 98% or 99% sequence identity thereto; the sequence set forth as nucleotides 2710-6009 of SEQ ID NO:58 or a sequence having at least 90%, 91%, 92%, 93%, 94%, 95%, 96%, 97%, 98% or 99% sequence identity thereto; and the sequence set forth as nucleotides 2710-6006 of SEQ ID NO:59 or a sequence having at least 90%, 91%, 92%, 93%, 94%, 95%, 96%, 97%, 98% or 99% sequence identity thereto.

61-69. (canceled)

70. The lentiviral vector of claim 1, further comprising a polyadenylation signal downstream of the first nucleic acid and the modified HS4-650 insulator.

71. The lentiviral vector of claim 1, that is a plasmid or a viral particle.

72. (canceled)

73. (canceled)

74. A host cell, wherein the host cell comprises the lentiviral vector of claim 1 or has been transduced with the lentiviral vector of claim 71.

75. (canceled)

76. The host cell of claim 74, wherein the host cell is a hematopoietic stem cell (HSC).

77-82. (canceled)

83. A method of treating a subject with Wiskott-Aldrich Syndrome, comprising administering to the subject the host cell of claim 74.

84-87. (canceled)

88. The lentiviral vector of claim 1, comprising the sequence set forth in SEQ ID NO:84 or a sequence having at least 90%, 91%, 92%, 93%, 94%, 95%, 96%, 97%, 98% or 99% sequence identity thereto.
